(19) United States
(12) Patent Application Publication
(10) Pub. No.: US 2025/0026730 A1
(43) Pub. Date: Jan. 23, 2025

(54) HETEROCYCLIC COMPOUND AND ORGANIC LIGHT EMITTING DEVICE COMPRISING THE SAME

(71) Applicant: LT MATERIALS CO., LTD., Yongin-si, Gyeonggi-do (KR)

(72) Inventors: Dong Jin LEE, Yongin-si (KR); GWANG IL DONG, Yongin-si (KR); Won Jang JEONG, Yongin-si (KR); Dong Jun KIM, Yongin-si (KR)

(73) Assignee: LT MATERIALS CO., LTD., Yongin-si, Gyeonggi-do (KR)

(21) Appl. No.: 18/290,481

(22) PCT Filed: Jul. 13, 2022
(86) PCT No.: PCT/KR2022/010207

§ 371 Date: Nov. 14, 2023

(30) Foreign Application Priority Data
Aug. 24, 2021 (KR) 10-2021-0111937

(51) Int. Cl. C07D 313/10; C07B 59/00; C07D 403/12; C07D 409/12; H10K 50/15; H10K 50/18; H10K 85/60

(52) U.S. Cl. CPC C07D 313/10; C07B 59/004; C07D 403/12; C07D 409/12; H10K 50/15; H10K 50/181; H10K 85/615; H10K 85/633; H10K 85/653; H10K 85/6574; H10K 85/6576; C07B 2200/05

(57) ABSTRACT

The present specification relates to a heterocyclic compound represented by Chemical Formula 1 and an organic light emitting device comprising the same.

### TECHNICAL FIELD

This application claims the benefit of priority based on Korean Patent Application No. 10-2021-0111937, filed with the Korean Intellectual Property Office on Aug. 24, 2021, the entire contents of which are incorporated herein as part of the present specification.

The present invention relates to a heterocyclic compound and an organic light emitting device comprising the same.

### BACKGROUND ART

An organic light emitting device is a kind of self-emission type display device, and there are advantages in that it not only has a wide viewing angle and excellent contrast, but also has fast response speed.

The organic light emitting device has a structure in which an organic thin film is disposed between two electrodes. When a voltage is applied to the organic light emitting device having such a structure, electrons and holes injected from the two electrodes combine in the organic thin film to form a pair, and then emit light while being disappeared. The organic thin film may be composed of a single layer or multiple layers as needed.

A material of the organic thin film may have a light emitting function as needed. For example, as a material of the organic thin film, compounds capable of forming a light emitting layer themselves alone may be used, or compounds capable of performing a role of a host or a dopant of a host-dopant based light emitting layer may also be used. In addition thereto, compounds capable of performing roles of hole injection, hole transport, electron blocking, hole blocking, electron transport, electron injection, and the like may also be used as a material of the organic thin film.

In order to improve performance, lifetime, or efficiency of an organic light emitting device, development of an organic thin film material has been continuously required.

### PRIOR ART DOCUMENTS

### Patent Documents

(Patent Document 1) U.S. Pat. No. 4,356,429

### DISCLOSURE

### Technical Problem

An object of the present disclosure is to provide a heterocyclic compound and an organic light emitting device comprising the same.

### Technical Solution

The present disclosure provides a heterocyclic compound represented by Chemical Formula 1 below.

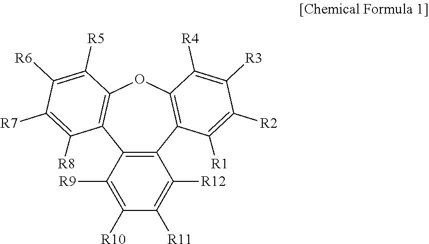

in Chemical Formula 1,
R1 to R12 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or to C60 alkoxy group; a substituted or unsubstituted C1 unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O) A101A102; —SiA101A102A103; and a group represented by Chemical Formula 2 below, or two or more groups adjacent to each other bond to each other to form a substituted or unsubstituted C6 to C60 aromatic hydrocarbon ring or a substituted or unsubstituted C2 to C60 heterocycle, and wherein A101, A102, and A103 are the same as or different from each other and are each independently a substituted or unsubstituted C1 to C60 alkyl group; a substituted or unsubstituted C6 to C60 aryl group; or a substituted or unsubstituted C2 to C60 heteroaryl group,

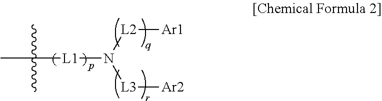

in Chemical Formula 2,
L1, L2, and L3 are the same as or different from each other, and are each independently a single bond; a substituted or unsubstituted C6 to C60 arylene group; or a substituted or unsubstituted C2 to C60 heteroarylene group,
Ar1 and Ar2 are the same as or different from each other, and are each independently a substituted or unsubstituted C6 to C60 aryl group; or a substituted or unsubstituted C2 to C60 heteroaryl group, and
p, q, and r are the same as or different from each other, and are each independently integers of 0 to 3.

Furthermore, the present disclosure provides an organic light emitting device comprising: a first electrode; a second electrode provided to face the first electrode; and one or more organic material layers provided between the first electrode and the second electrode, wherein at least one of the one or more layers of the organic material layers comprise the heterocyclic compound represented by Chemical Formula 1 above.

Furthermore, the present disclosure provides an organic light emitting device in which the organic material layers comprise a hole transport layer, and the hole transport layer contains the heterocyclic compound.

Furthermore, the present disclosure provides an organic light emitting device in which the organic material layers comprise an electron blocking layer, and the electron blocking layer contains the heterocyclic compound.

Furthermore, the present disclosure provides a method for manufacturing an organic light emitting device, the method comprising steps of: preparing a substrate; forming a first electrode on the substrate; forming one or more organic material layers on the first electrode; and forming a second electrode on the organic material layers, wherein the step of forming the organic material layers includes a step of forming one or more organic material layers using a composition for organic material layers of an organic light emitting device comprising the heterocyclic compound represented by Chemical Formula 1 above.

### Advantageous Effects

The heterocyclic compound according to one embodiment can be used as a material of organic material layers of an organic light emitting device. The heterocyclic compound can be used as a material for a hole injection layer, a hole transport layer, an electron blocking layer, a light emitting layer, an electron transport layer, a hole blocking layer, an electron injection layer, a charge generating layer, or the like in an organic light emitting device. Particularly, the heterocyclic compound represented by Chemical Formula 1 above can be used as a material for a hole transport layer or an electron blocking layer of an organic light emitting device. Specifically, the heterocyclic compound represented by Chemical Formula 1 above can be used alone or in combination with other compounds as a material for a hole transport layer or an electron blocking layer.

When the heterocyclic compound according to another embodiment is used as a hole transport layer and an electron blocking layer, it can enhance hole characteristics, can improve hole transport ability through adjustment of band gap and triplet energy level (T₁ level) values, can lower driving voltage of the organic light emitting device and improve light efficiency by increasing stability of the molecule, and can improve lifetime properties of the organic light emitting device by improved thermal stability of the compound.

### DESCRIPTION OF DRAWINGS

FIGS. 1 to 3 are views schematically showing a stacked structure of an organic light emitting device according to one embodiment of the present disclosure, respectively.

### BEST MODE FOR CARRYING OUT THE INVENTION

Hereinafter, the present disclosure will be described in detail.

In the present specification, the term “substitution” means that a hydrogen atom bonded to a carbon atom of a compound is changed to another substituent, and the position to be substituted is not limited as long as it is a position at which the hydrogen atom is substituted, that is, a position where the substituent is substitutable, and when two or more substituents are substituted, the two or more substituents may be the same as or different from each other.

In the present specification, the term “substituted or unsubstituted” means that it is substituted with one or more substituents selected from the group consisting of deuterium; halogen; a cyano group; C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C₁-C₂₀ alkylamine; C6 to C60 monocyclic or polycyclic arylamine; and C2 to C60 monocyclic or polycyclic heteroarylamine or is unsubstituted, or it is substituted with a substituent linking more substituents selected from among the substituents illustrated above or being unsubstituted. R, R′, and R″ may be the same as or different from each other, and may each independently be a substituent consisting of at least one of hydrogen; deuterium; a halogen group; an alkyl group; an alkenyl group; an alkoxy group; a cycloalkyl group; an aryl group; and a heterocyclic group.

In the present specification, the halogen may be fluorine, chlorine, bromine, or iodine.

In the present specification, the alkyl group may include a linear or branched chain having 1 to 60 carbon atoms, and may be further substituted by other substituents. The number of carbon atoms of the alkyl group may be 1 to 60, specifically 1 to 40, and more specifically 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl a 1-methylhexyl group, group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group, and the like, but are not limited thereto.

In the present specification, the alkenyl group may include a linear or branched chain having 2 to 60 carbon atoms, and may be further substituted by other substituents. The number of carbon atoms of the alkenyl group may be 2 to 60, specifically 2 to 40, and more specifically 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, and the like, but are not limited thereto.

In the present specification, the alkynyl group may include a linear or branched chain having 2 to 60 carbon atoms, and may be further substituted by other substituents. The number of carbon atoms of the alkynyl group may be 2 to 60, specifically 2 to 40, and more specifically 2 to 20.

In the present specification, the alkoxy group may be a linear, branched, or cyclic chain. The number of carbon atoms of the alkoxy group is not particularly limited, but is preferably 1 to 20. Specific examples thereof may include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group may include a monocyclic or polycyclic ring having 3 to 60 carbon atoms, and may be further substituted by other substituents. Herein, the polycyclic ring refers to a group in which a cycloalkyl group is directly linked to or condensed with other ring groups. Herein, the other ring groups may be a cycloalkyl group, but may also be different types of ring groups such as a heterocycloalkyl group, an aryl group, a heteroaryl group, and the like. The number of carbon atoms of the cycloalkyl group may be 3 to 60, specifically 3 to 40, and more specifically 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group may contain O, S, Se, N, or Si as a heteroatom, may include a monocyclic or polycyclic ring having 2 to 60 carbon atoms, and may be further substituted by other substituents. Herein, the polycyclic ring refers to a group in which a heterocycloalkyl group is directly linked to or condensed with other ring groups. Herein, the other ring groups may be a heterocycloalkyl group, but may also be different types of ring groups such as a cycloalkyl group, an aryl group, and a heteroaryl group. The number of carbon atoms of the heterocycloalkyl group may be 2 to 60, specifically 2 to 40, and more specifically 3 to 20.

In the present specification, the aryl group may include a monocyclic or polycyclic ring having 6 to 60 carbon atoms, and may be further substituted by other substituents. Herein, the polycyclic ring means a group in which an aryl group is directly linked to or condensed with other ring groups. Herein, the other ring groups may be an aryl group, but may also be different types of ring such groups as a cycloalkyl group, a heterocycloalkyl group, and a heteroaryl group. The aryl group includes a spiro group. The number of carbon atoms of the aryl group may be 6 to 60, specifically 6 to 40, and more specifically 6 to 25. Specific examples of the aryl group may include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, condensed ring groups thereof, and the like, but are not limited thereto.

In the present specification, the phosphine oxide group may be represented by —P(═O)A101A102, and A101 and A102 may be the same as or different from each other and may be each independently a substituent consisting of at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and the examples described above may be used as the aryl group. Examples of the phosphine oxide group may include a diphenyl phosphine oxide group, a dinaphthyl phosphine oxide group, and the like, but are not limited thereto.

In the present specification, the silyl group may be a substituent containing Si and having the Si atom directly linked thereto as a radical and may be represented by —SiA104A105A106, and A104 to A106 may be the same as or different from each other, and may be each independently a substituent consisting of at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, the fluorenyl group may be substituted, and adjacent substituents may bond to each other to form a ring.

When the fluorenyl group is substituted, examples of the substituted fluorenyl group may include

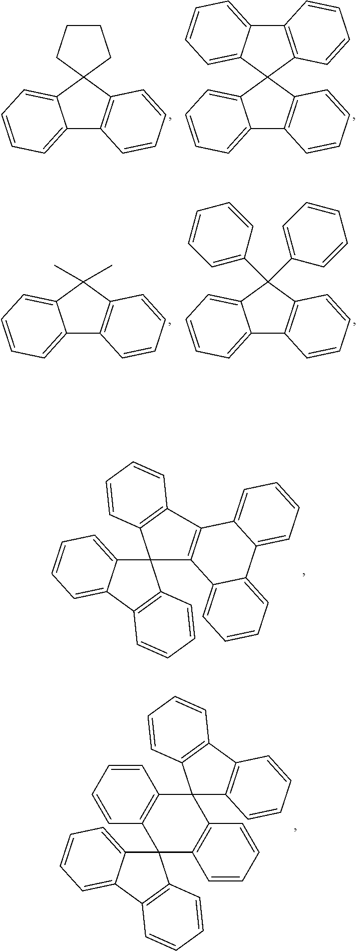

and the like, but are not limited thereto.

In the present specification, the heteroaryl group may contain S, O, Se, N, or Si as a heteroatom, may include a monocyclic or polycyclic ring having 2 to 60 carbon atoms, and may be further substituted by other substituents. Herein, the polycyclic ring refers to a group in which a heteroaryl group is directly linked to or condensed with other ring groups. Herein, the other ring groups may be a heteroaryl group, but may also be different types of ring groups such as a cycloalkyl group, a heterocycloalkyl group, and an aryl group. The number of carbon atoms of the heteroaryl group may be 2 to 60, specifically 2 to 40, and more specifically 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxinyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diazanaphthalenyl group, a triazaindenyl group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a spirobi (dibenzosilole) group, a dibenzosilole group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms of the amine group is not particularly limited, but is preferably 1 to 30. Specific examples of the amine group may include a methylamine group, a dimethylamine group, an ethylamine group, a diethylamine group, a phenylamine group, a naphthylamine group, a dibiphenylamine group, a biphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the arylene group means one having two bonding positions in the aryl group, that is, a divalent group. The description of the aryl group described above may be applied except that each of these is a divalent group. Further, the heteroarylene group means one having two bonding positions in the heteroaryl group, that is, a divalent group. The description of the heteroaryl group described above may be applied except that each of these is a divalent group.

In the present specification, an “adjacent” group may mean a substituent substituted on an atom directly linked to an atom in which the corresponding substituent is substituted, substituent sterically most closely positioned to the corresponding substituent, or another substituent substituted on an atom in which the corresponding substituent is substituted. For example, two substituents substituted at ortho positions in a benzene ring and two substituents substituted at the same carbon in an aliphatic ring may be interpreted as groups “adjacent” to each other.

In the present disclosure, a “case of a substituent being not indicated in a chemical formula or compound structure” means that a hydrogen atom is bonded to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions that may come as a substituent are all hydrogen or deuterium. In other words, since deuterium is an isotope of hydrogen, some hydrogen atoms may be deuterium that is an isotope, and the content of deuterium may be 0% to 100% at this time.

In one embodiment of the present disclosure, in a “case of a substituent being not indicated in a chemical formula or compound structure”, hydrogen and deuterium may be mixed and used in compounds when deuterium is not explicitly excluded such as “a deuterium content being 0%”, “a hydrogen content being 100%”, or “substituents being all hydrogen”.

In one embodiment of the present disclosure, deuterium is one of isotopes of hydrogen, is an element having deuteron consisting of one proton and one neutron as a nucleus, and may be expressed as hydrogen-2, and the elemental symbol may also be written as D or ²H.

In one embodiment of the present disclosure, although isotopes have the same atomic number (Z), isotopes meaning atoms having different mass numbers (A) have the same number of protons, but they may also be interpreted as elements with different numbers of neutrons.

In one embodiment of the present disclosure, when the total number of substituents that a basic compound may have is defined as T₁, and the number of specific substituents among them is defined as T₂, the meaning of a content T % of the specific substituents may be defined as T₂/T₁×100=T %.

In other words, in one example, having a deuterium content of 20% in a phenyl group represented by

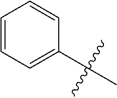 [SMILES: *c1ccccc1]

may mean that the total number of substituents that the phenyl group may have is 5 (T₁ in Equation), and the number of deuteriums among them is 1 (T₂ in Equation). In other words, having a deuterium content of 20% in the phenyl group may be represented by Structural Formulas as below.

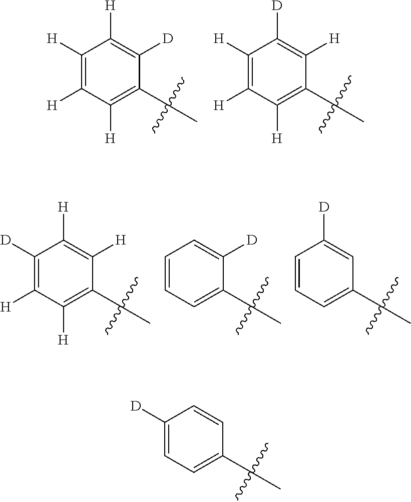

Further, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound containing an aromatic ring consisting of C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene, and the like, but are not limited thereto, and include all aromatic hydrocarbon ring compounds known in the art as ones satisfying the aforementioned number of carbon atoms.

The present disclosure provides a heterocyclic compound represented by the following Chemical Formula 1:

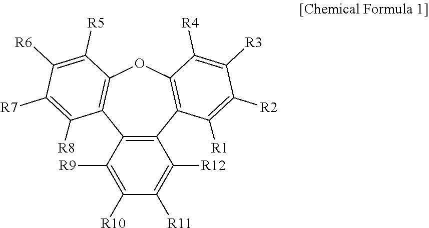

wherein,
R1 to R12 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and a group represented by Chemical Formula 2 below, or two or more groups adjacent to each other bond to each other to form a substituted or unsubstituted C6 to C60 aromatic hydrocarbon ring or a substituted or unsubstituted C2 to C60 heterocycle, and wherein A101, A102, and A103 are the same as or different from each other and are each independently a substituted or unsubstituted C1 to C60 alkyl group; a substituted or unsubstituted C6 to C60 aryl group; or a substituted or unsubstituted C2 to C60 heteroaryl group,

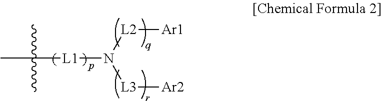

wherein,
L1, L2, and L3 are the same as or different from each other, and are each independently a single bond; a substituted or unsubstituted C6 to C60 arylene group; or a substituted or unsubstituted C2 to C60 heteroarylene group,
Ar1 and Ar2 are the same as or different from each other, and are each independently a substituted or unsubstituted C6 to C60 aryl group; or a substituted or unsubstituted C2 to C60 heteroaryl group, and
p, q, and r are the same as or different from each other, and are each independently integers of 0 to 3.

The substituted or unsubstituted C6 to C60 aromatic hydrocarbon ring or the substituted or unsubstituted C2 to C60 heterocycle that the adjacent groups may form may be a substituted or unsubstituted C6 to C60 aryl group; or a substituted or unsubstituted C2 to C60 heteroaryl group except that it is not a monovalent group.

In one embodiment of the present disclosure, at least one of R1 to R8 may be a group represented by Chemical Formula 2 above.

In another embodiment of the present disclosure, at least one of R1 to R4 may be a group represented by Chemical Formula 2 above, R5 to R12 may be the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic hydrocarbon ring or a substituted or unsubstituted C2 to C60 heterocycle.

In another embodiment of the present disclosure, at least one of R1 to R4 may be a group represented by Chemical Formula 2 above, R5 to R12 may be the same as or different from each other, and are each independently selected from the group consisting of hydrogen; deuterium; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic hydrocarbon ring or a substituted or unsubstituted C2 to C60 heterocycle.

In another embodiment of the present disclosure, at least one of R1 to R4 may be a group represented by Chemical Formula 2 above, and at least one of R5 to R12 may be a substituted or unsubstituted C6 to C60 aryl group.

In another embodiment of the present disclosure, at least one of R5 to R8 may be a group represented by Chemical Formula 2 above, and at least one of R1 to R4 and R9 to R12 may be a substituted or unsubstituted C6 to C60 aryl group.

In another embodiment of the present disclosure, one of R1 to R4 or R5 to R8 may be a group represented by Chemical Formula 2 above, and the rest may be hydrogen, deuterium, or substituted or unsubstituted C6 to C60 aryl group.

In another embodiment of the present disclosure, one of R1 to R4 may be a group represented by Chemical Formula 2 above, and the rest may be hydrogen, deuterium, or a substituted or unsubstituted C6 to C60 aryl group.

In another embodiment of the present disclosure, one of R5 to R8 may be a group represented by Chemical Formula 2 above, and the rest may be hydrogen, deuterium, or a substituted or unsubstituted C6 to C60 aryl group.

In another embodiment of the present disclosure, at least one of R1 to R4 may be a group represented by Chemical Formula 2 above, at least one of R5 to R8 may be a substituted or unsubstituted C6 to C60 aryl group, and at least one of R9 to R12 may be a substituted or unsubstituted C6 to C60 aryl group.

In another embodiment of the present disclosure, at least one of R5 to R8 may be a group represented by Chemical Formula 2 above, at least one of R1 to R4 may be a substituted or unsubstituted C6 to C60 aryl group, and at least one of R9 to R12 may be a substituted or unsubstituted C6 to C60 aryl group.

In another embodiment of the present disclosure, R1 to R12 may be each independently hydrogen; deuterium; groups represented by the following Chemical Formulas B-1 to B-3, and the like, but are not limited to these examples:

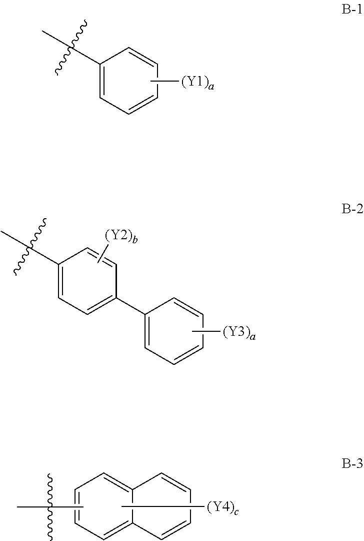

in Chemical Formulas B-1 to B-3,
Y1 to Y4 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or group; a substituted or unsubstituted C2 to C60 alkynyl unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and —NA101A102, and wherein A101, A102, and A103 are the same as or different from each other and each independently a substituted or unsubstituted C1 to C60 alkyl group; a substituted or unsubstituted C6 to C60 aryl group; or a substituted or unsubstituted C6 to C60 heteroaryl group, and
a to c are the same as or different from each other, and are each independently integers of 0 to 7.

In one embodiment of the present disclosure, L1, L2, and L3 may be the same as or different from each other, and are each independently a single bond; a substituted or unsubstituted C6 to C60 arylene group; or a substituted or unsubstituted C2 to C60 heteroarylene group.

In another embodiment of the present disclosure, L1, L2, and L3 may be the same as or different from each other, and are each independently a single bond; a substituted or unsubstituted C6 to C30 arylene group; or a substituted or unsubstituted C2 to C30 heteroarylene group.

In another embodiment of the present disclosure, L1, L2, and L3 may be the same as or different from each other, and are each independently a single bond; a substituted or unsubstituted C6 to C20 arylene group; or a substituted or unsubstituted C2 to C20 heteroarylene group.

In another embodiment of the present disclosure, L1, L2, and L3 may be the same as or different from each other, and are each independently a single bond; Chemical Formula C-1; Chemical Formula C-2; Chemical Formula C-3, and the like, but are not limited to these examples:

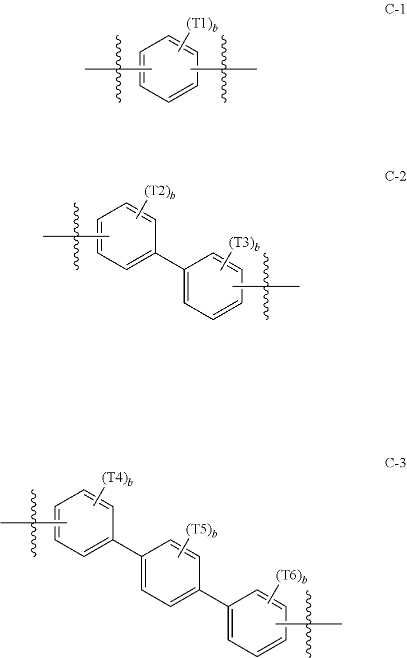

in Chemical Formulas C-1 to C-3,
T₁ to T₆ are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and —NA101A102, and wherein A101, A102, and A103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
b are the same as or different from each other, and are each independently integers of 0 to 4.

In one embodiment of the present disclosure, Ar1 and Ar2 may be the same as or different from each other, and are each independently a substituted or unsubstituted C6 to C60 aryl group; or a substituted or unsubstituted C2 to C60 heteroaryl group.

In another embodiment of the present disclosure, Ar1 and Ar2 may be the same as or different from each other, and are each independently a substituted or unsubstituted C6 to C30 aryl group; or a substituted or unsubstituted C2 to C30 heteroaryl group.

In another embodiment of the present disclosure, Ar1 and Ar2 may be the same as or different from each other, and are each independently the following Chemical Formulas A-1 to A-9, and the like, but are not limited to these examples:

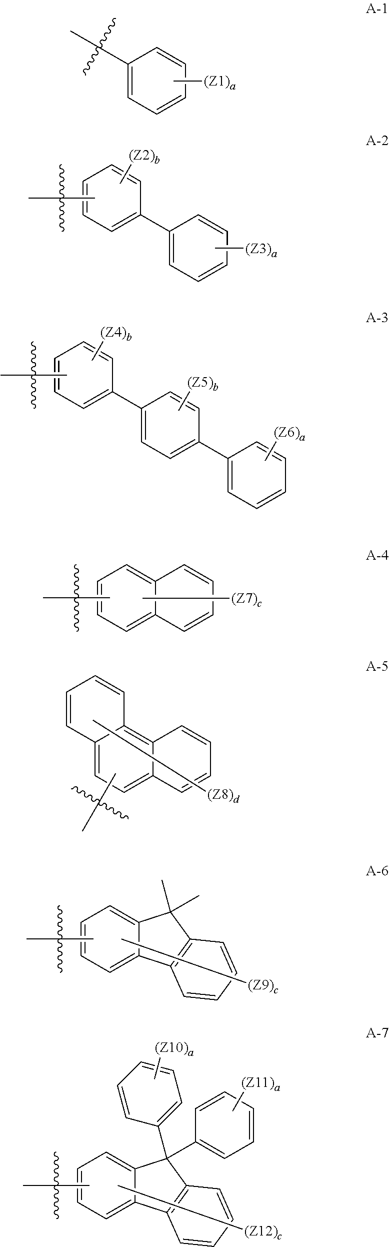

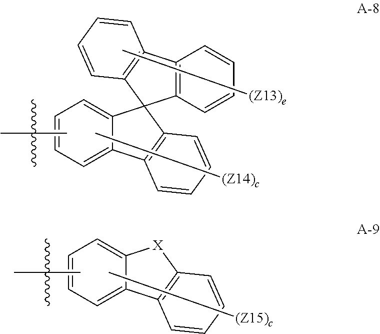

in Chemical Formulas A-1 to A-9,
X is O or S,
Z1 to Z15 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and —NA101A102, and wherein A101, A102, and A103 are the same as or different from each other and each independently a substituted or unsubstituted C1 to C60 alkyl group; a substituted or unsubstituted C6 to C60 aryl group; or a substituted or unsubstituted C2 to C60 heteroaryl group, and
a are the same as or different from each other, and each independently integers of 0 to 5; b are the same as or different from each other, and each independently integers of 0 to 4; c are the same as or different from each other, and each independently integers of 0 to 7; d are the same as or different from each other, and each independently integers of 0 to 9; and e are the same as or different from each other, and each independently integers of 0 to 8.

The compound of Chemical Formula 1 of the present disclosure provides a heterocyclic compound represented by any one of the following Chemical Formula 1-1, Chemical Formula 1-2, or Chemical Formula 1-3:

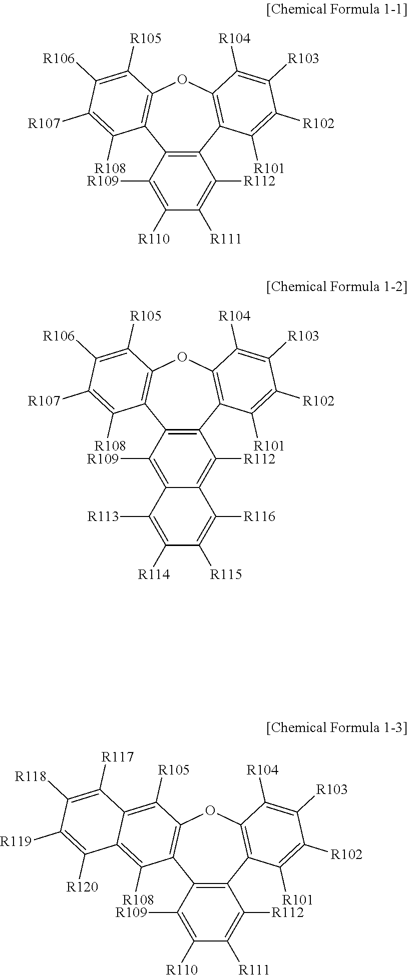

in Chemical Formulas 1-1 to 1-3,
R101 to R120 may be the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and a group represented by Chemical Formula 2 below, and wherein A101, A102, and A103 may be the same as or different from each other and are each independently a substituted or unsubstituted C1 to C60 alkyl group; a substituted or unsubstituted C6 to C60 aryl group; or a substituted or unsubstituted C2 to C60 heteroaryl group,

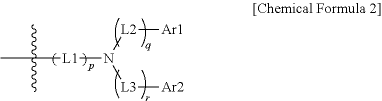

in Chemical Formula 2,
L1, L2, and L3 may be the same as or different from each other, and are each independently a single bond; a substituted or unsubstituted C6 to C60 arylene group; or a substituted or unsubstituted C2 to C60 heteroarylene group,
Ar1 and Ar2 may be the same as or different from each other, and each independently a substituted or unsubstituted C6 to C60 aryl group; or a substituted or unsubstituted C2 to C60 heteroaryl group,
p, q, and r may be the same as or different from each other, and are each independently integers of 0 to 3,
in Chemical Formula 1-1 above, when at least one of R101 to R104 is a group represented by Chemical Formula 2 above, at least one of R105 to R112 may be a substituted or unsubstituted C6 to C60 aryl group,
in Chemical Formula 1-2 above, when at least one of R101 to R104 is a group represented by Chemical Formula 2 above, at least one of R105 to R109 and R112 to R116 may be a substituted or unsubstituted C6 to C60 aryl group, and
in Chemical Formula 1-3 above, when at least one of R101 to R104 is a group represented by Chemical Formula 2 above, at least one of R105, R108 to R112, and R117 to R120 may be a substituted or unsubstituted C6 to C60 aryl group.

In one embodiment of the present disclosure, at least one of R101 to R108 may be a group represented by Chemical Formula 2 above.

In another embodiment of the present disclosure, at least one of R101 to R104 may be a group represented by Chemical Formula 2 above, and R105 to R120 may be the same as or different from each other, and are each independently selected from the group consisting of hydrogen; deuterium; halogen; a cyano group; a substituted or unsubstituted C1 to C60 alkyl a group; substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

In another embodiment of the present disclosure, at least one of R101 to R104 may be a group represented by Chemical Formula 2 above, and R105 to R120 may be the same as or different from each other, and are each independently selected from the group consisting of hydrogen; deuterium; a substituted or unsubstituted C6 to C60 aryl group; and a substituted or unsubstituted C2 to C60 heteroaryl group.

In another embodiment of the present disclosure, in Chemical Formula 1-1 above, at least one of R101 to R104 may be a group represented by Chemical Formula 2 above, and at least one of R105 to R112 may be a substituted or unsubstituted C6 to C60 aryl group.

In another embodiment of the present disclosure, in Chemical Formula 1-1 above, at least one of R105 to R108 may be a group represented by Chemical Formula 2 above, and at least one of R101 to R104 and R109 to R112 may be a substituted or unsubstituted C6 to C60 aryl group.

In another embodiment of the present disclosure, in Chemical Formula 1-2 above, at least one of R101 to R104 may be a group represented by Chemical Formula 2 above, and at least one of R105 to R109 and R112 to R116 may be a substituted or unsubstituted C6 to C60 aryl group.

In another embodiment of the present disclosure, in Chemical Formula 1-2 above, at least one of R105 to R108 may be a group represented by Chemical Formula 2 above, and at least one of R101 to R104, R109, and R112 to R116 may be a substituted or unsubstituted C6 to C60 aryl group.

In another embodiment of the present disclosure, in Chemical Formula 1-2 above, at least one of R101 to R104 may be a group represented by Chemical Formula 2 above, and at least one of R105 to R108 may be a substituted or unsubstituted C6 to C60 aryl group.

In another embodiment of the present disclosure, in Chemical Formula 1-3 above, at least one of R101 to R104 may be a group represented by Chemical Formula 2 above, and at least one of R105, R108 to R112, and R117 to R120 may be a substituted or unsubstituted C6 to C60 aryl group.

In another embodiment of the present disclosure, in Chemical Formula 1-3 above, at least one of R101 to R104 may be a group represented by Chemical Formula 2 above, and at least one of R109 to R112 may be a substituted or unsubstituted C6 to C60 aryl group.

In another embodiment of the present disclosure, in Chemical Formula 1-1 above, at least one of R101 to R104 may be a group represented by Chemical Formula 2 above, and at least one of R105 to R108 may be a substituted or unsubstituted C6 to C60 aryl group, and at least one of R109 to R112 may be a substituted or unsubstituted C6 to C60 aryl group.

In another embodiment of the present disclosure, in Chemical Formula 1-1 above, at least one of R105 to R108 may be a group represented by Chemical Formula 2 above, at least one of R101 to R104 may be a substituted or unsubstituted C6 to C60 aryl group, and at least one of R109 to R112 may be a substituted or unsubstituted C6 to C60 aryl group.

In another embodiment of the present disclosure, in Chemical Formula 1-2 above, at least one of R101 to R104 may be a group represented by Chemical Formula 2 above, at least one of R105 to R108 may be a substituted or unsubstituted C6 to C60 aryl group, and at least one of R109 and R112 to R116 may be a substituted or unsubstituted C6 to C60 aryl group.

In another embodiment of the present disclosure, in Chemical Formula 1-2 above, at least one of R105 to R108 may be a group represented by Chemical Formula 2 above, at least one of R101 to R104 may be a substituted or unsubstituted C6 to C60 aryl group, and at least one of R109 and R112 to R116 may be a substituted or unsubstituted C6 to C60 aryl group.

In another embodiment of the present disclosure, in Chemical Formula 1-3 above, at least one of R101 to R104 may be a group represented by Chemical Formula 2 above, at least one of R105, R108, and R117 to R120 may be a substituted or unsubstituted C6 to C60 aryl group, and at least one of R109 to R112 may be a substituted or unsubstituted C6 to C60 aryl group.

In another embodiment of the present disclosure, R101 to R120 may be each independently hydrogen; deuterium; groups represented by the following Chemical Formulas B-1 to B-3, and the like, but are not limited to these examples:

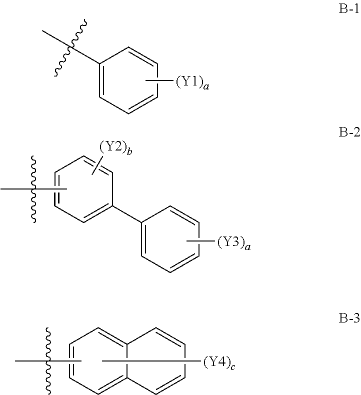

in Chemical Formulas B-1 to B-3,
Y1 to Y4 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and —NA101A102, and wherein A101, A102, and A103 are the same as or different from each other and each independently a substituted or unsubstituted C1 to C60 alkyl group; a substituted or unsubstituted C6 to C60 aryl group; or a substituted or unsubstituted C6 to C60 heteroaryl group, and
a to c are the same as or different from each other, and are each independently integers of 0 to 7.

In the compounds of Chemical Formula 1-1, Chemical Formula 1-2, and Chemical Formula 1-3 of the present disclosure, L1, L2, L3, Ar1, Ar2, p, q, and r are the same as defined in the compound of Chemical Formula 1 above.

In one embodiment of the present disclosure, the heterocyclic compound represented by Chemical Formula 1 above may not contain deuterium as a substituent, or the content of deuterium based on the total number of hydrogen atoms and deuterium atoms may be 1% to 100%.

In another embodiment of the present disclosure, the heterocyclic compound represented by Chemical Formula 1 above may not contain deuterium as a substituent, or the content of deuterium based on the total number of hydrogen atoms and deuterium atoms may be 10% to 100%.

In another embodiment of the present disclosure, the heterocyclic compound represented by Chemical Formula 1 above may not contain deuterium as a substituent, or the content of deuterium based on the total number of hydrogen atoms and deuterium atoms may be 20% to 90%.

In another embodiment of the present disclosure, the heterocyclic compound represented by Chemical Formula 1 above may not contain deuterium as a substituent, or the content of deuterium based on the total number of hydrogen atoms and deuterium atoms may be 30% to 80%.

For example, the heterocyclic compound represented by Chemical Formula 1 above may not contain deuterium as a substituent, or the content of deuterium may be more than 0%, 5% or more, 10% or more, 15% or more, 20% or more, 25% or more, 30% or more, 35% or more, 40% or more, 45% or more, or 50% or more, and may be 100% or less, 95% or less, 90% or less, 85% or less, 80% or less, 75% or less, 70% or less, 65% or less, or 60% or less based on the total number of hydrogen atoms and deuterium atoms.

In one embodiment of the present disclosure, the heterocyclic compound represented by Chemical Formula 1 above may be one or more selected from the compounds below.

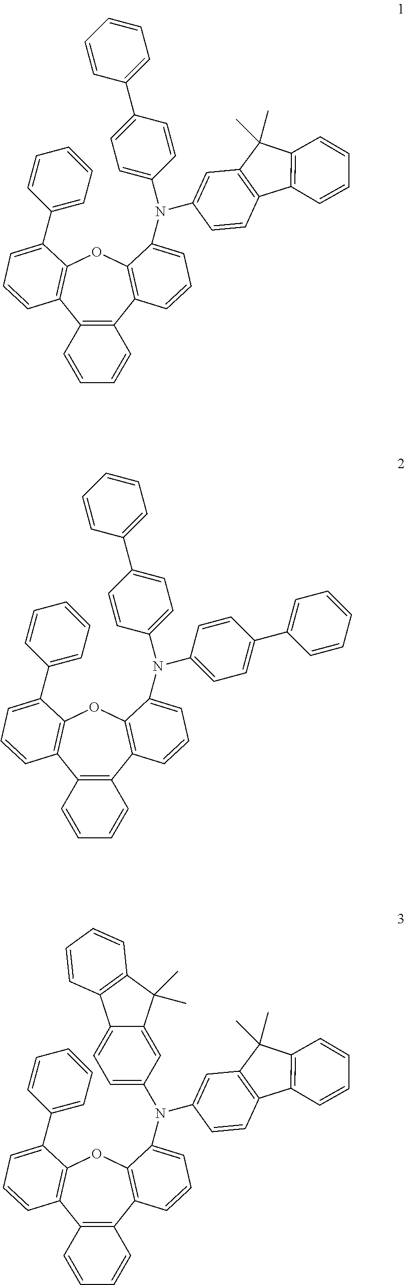 [SMILES: CC1(C)c2ccccc2-c2ccc(N(c3ccc(-c4ccccc4)cc3)c3cccc4c3Oc3c(-c5ccccc5)cccc3-c3ccccc3-4)cc21 ; c1ccc(-c2ccc(N(c3ccc(-c4ccccc4)cc3)c3cccc4c3Oc3c(-c5ccccc5)cccc3-c3ccccc3-4)cc2)cc1 ; CC1(C)c2ccccc2-c2ccc(N(c3ccc4c(c3)C(C)(C)c3ccccc3-4)c3cccc4c3Oc3c(-c5ccccc5)cccc3-c3ccccc3-4)cc21]

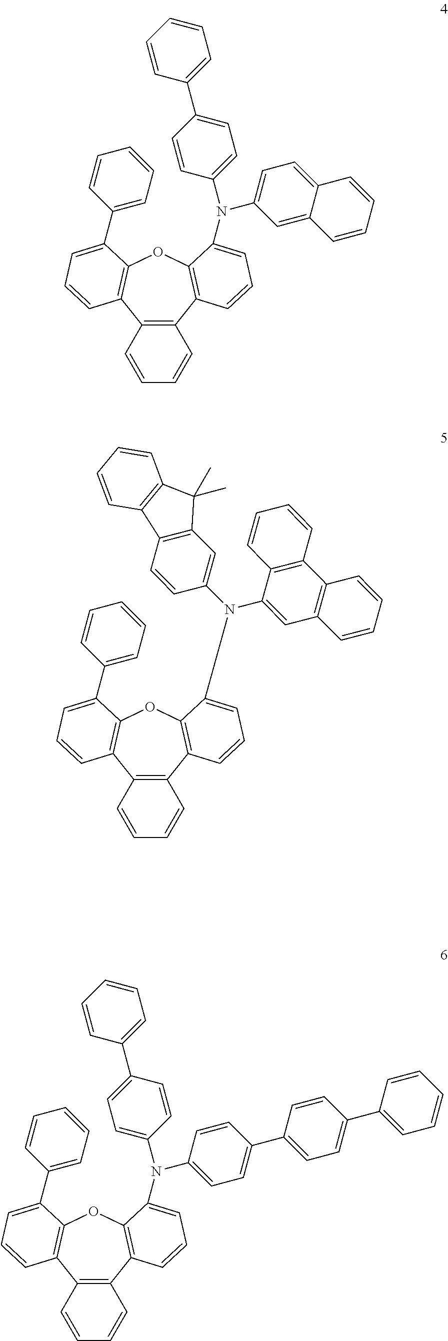 [SMILES: c1ccc(-c2ccc(N(c3ccc4ccccc4c3)c3cccc4c3Oc3c(-c5ccccc5)cccc3-c3ccccc3-4)cc2)cc1 ; CC1(C)c2ccccc2-c2ccc(N(c3cccc4c3Oc3c(-c5ccccc5)cccc3-c3ccccc3-4)c3cc4ccccc4c4ccccc34)cc21 ; c1ccc(-c2ccc(-c3ccc(N(c4ccc(-c5ccccc5)cc4)c4cccc5c4Oc4c(-c6ccccc6)cccc4-c4ccccc4-5)cc3)cc2)cc1]

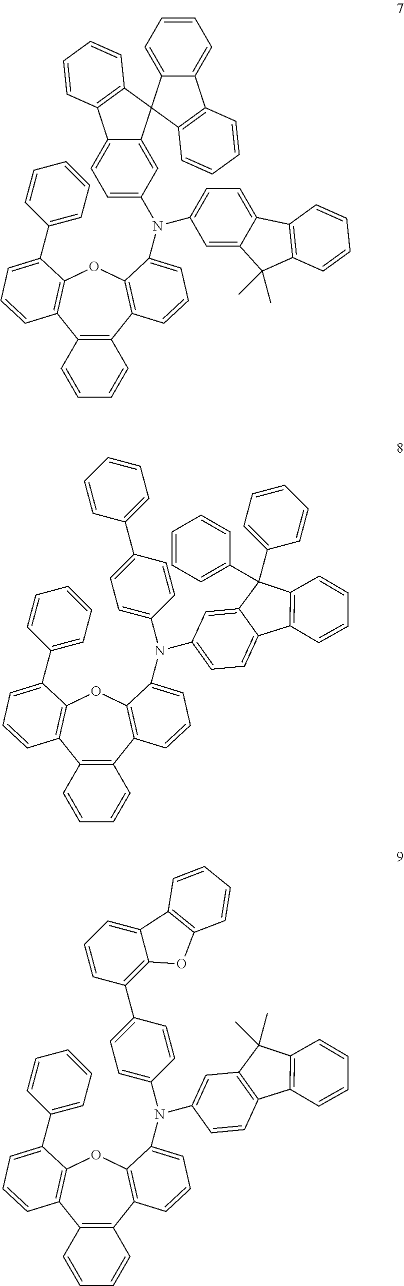 [SMILES: CC1(C)c2ccccc2-c2ccc(N(c3ccc4c(c3)C3(c5ccccc5-c5ccccc53)c3ccccc3-4)c3cccc4c3Oc3c(-c5ccccc5)cccc3-c3ccccc3-4)cc21 ; c1ccc(-c2ccc(N(c3ccc4c(c3)C(c3ccccc3)(c3ccccc3)c3ccccc3-4)c3cccc4c3Oc3c(-c5ccccc5)cccc3-c3ccccc3-4)cc2)cc1 ; CC1(C)c2ccccc2-c2ccc(N(c3ccc(-c4cccc5c4oc4ccccc45)cc3)c3cccc4c3Oc3c(-c5ccccc5)cccc3-c3ccccc3-4)cc21]

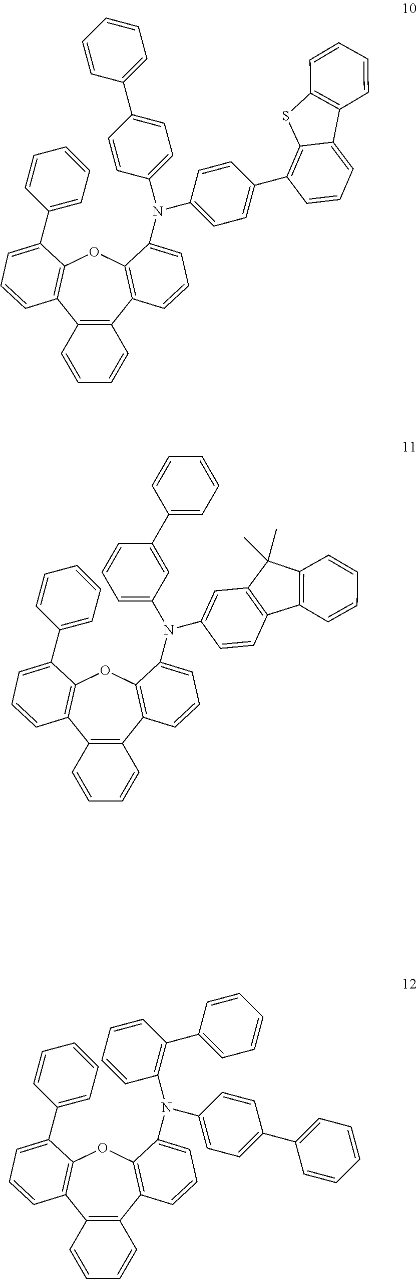 [SMILES: c1ccc(-c2ccc(N(c3ccc(-c4cccc5c4sc4ccccc45)cc3)c3cccc4c3Oc3c(-c5ccccc5)cccc3-c3ccccc3-4)cc2)cc1 ; CC1(C)c2ccccc2-c2ccc(N(c3cccc(-c4ccccc4)c3)c3cccc4c3Oc3c(-c5ccccc5)cccc3-c3ccccc3-4)cc21 ; c1ccc(-c2ccc(N(c3ccccc3-c3ccccc3)c3cccc4c3Oc3c(-c5ccccc5)cccc3-c3ccccc3-4)cc2)cc1]

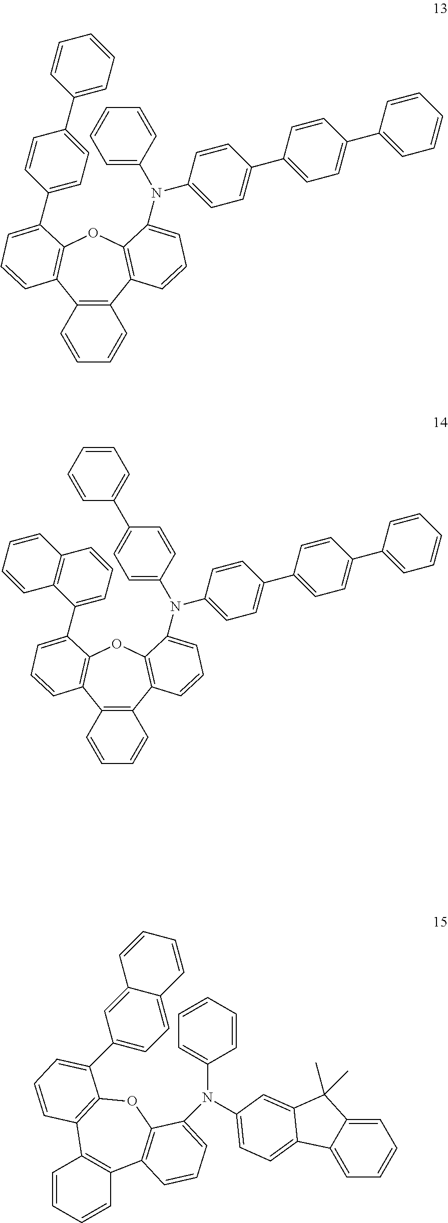 [SMILES: c1ccc(-c2ccc(-c3ccc(N(c4ccccc4)c4cccc5c4Oc4c(-c6ccc(-c7ccccc7)cc6)cccc4-c4ccccc4-5)cc3)cc2)cc1 ; c1ccc(-c2ccc(-c3ccc(N(c4ccc(-c5ccccc5)cc4)c4cccc5c4Oc4c(cccc4-c4cccc6ccccc46)-c4ccccc4-5)cc3)cc2)cc1 ; CC1(C)c2ccccc2-c2ccc(N(c3ccccc3)c3cccc4c3Oc3c(-c5ccc6ccccc6c5)cccc3-c3ccccc3-4)cc21]

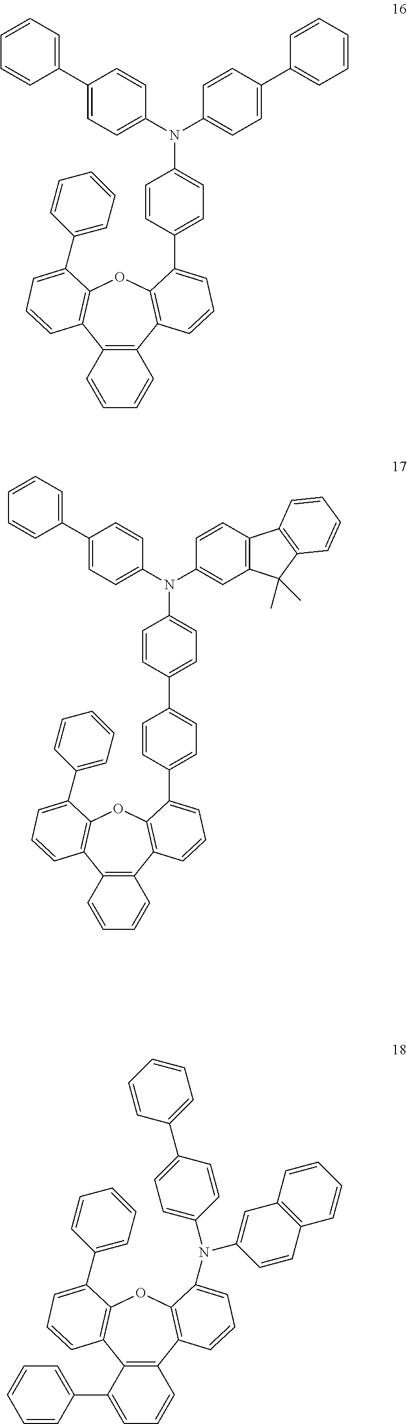 [SMILES: c1ccc(-c2ccc(N(c3ccc(-c4ccccc4)cc3)c3ccc(-c4cccc5c4Oc4c(-c6ccccc6)cccc4-c4ccccc4-5)cc3)cc2)cc1 ; CC1(C)c2ccccc2-c2ccc(N(c3ccc(-c4ccccc4)cc3)c3ccc(-c4ccc(-c5cccc6c5Oc5c(-c7ccccc7)cccc5-c5ccccc5-6)cc4)cc3)cc21 ; c1ccc(-c2ccc(N(c3ccc4ccccc4c3)c3cccc4c3Oc3c(-c5ccccc5)cccc3-c3c(-c5ccccc5)cccc3-4)cc2)cc1]

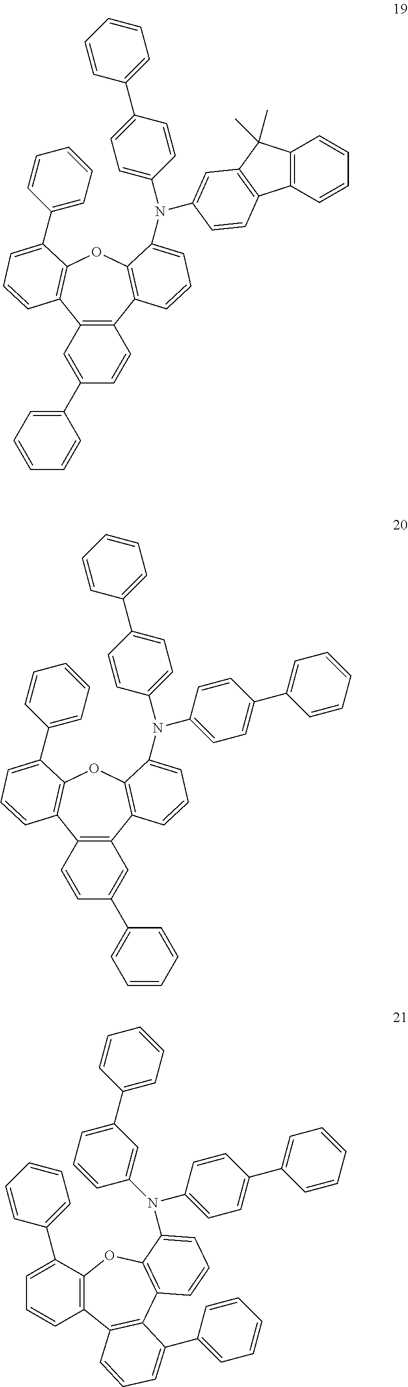

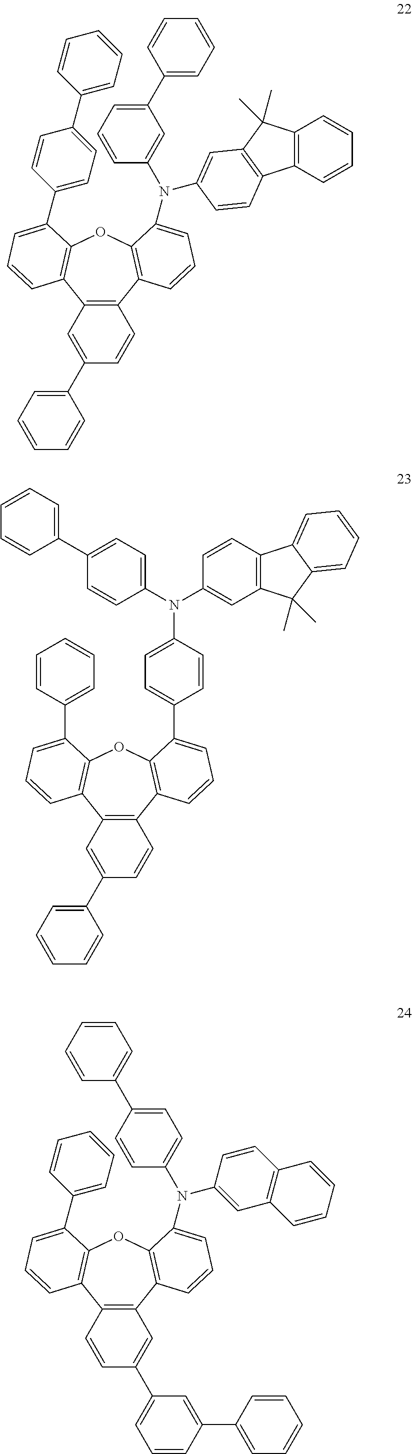

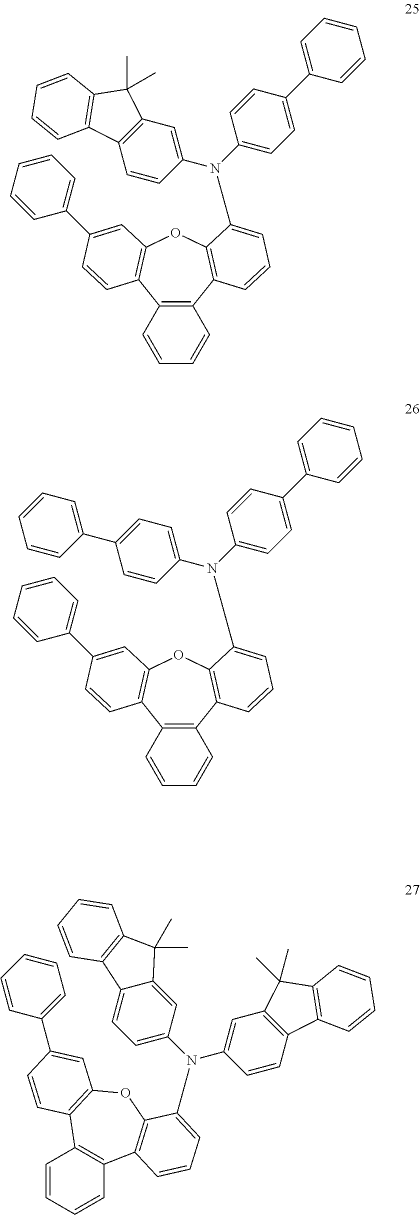 [SMILES: CC1(C)c2ccccc2-c2ccc(N(c3ccc(-c4ccccc4)cc3)c3cccc4c3Oc3cc(-c5ccccc5)ccc3-c3ccccc3-4)cc21 ; c1ccc(-c2ccc(N(c3ccc(-c4ccccc4)cc3)c3cccc4c3Oc3cc(-c5ccccc5)ccc3-c3ccccc3-4)cc2)cc1 ; CC1(C)c2ccccc2-c2ccc(N(c3ccc4c(c3)C(C)(C)c3ccccc3-4)c3cccc4c3Oc3cc(-c5ccccc5)ccc3-c3ccccc3-4)cc21]

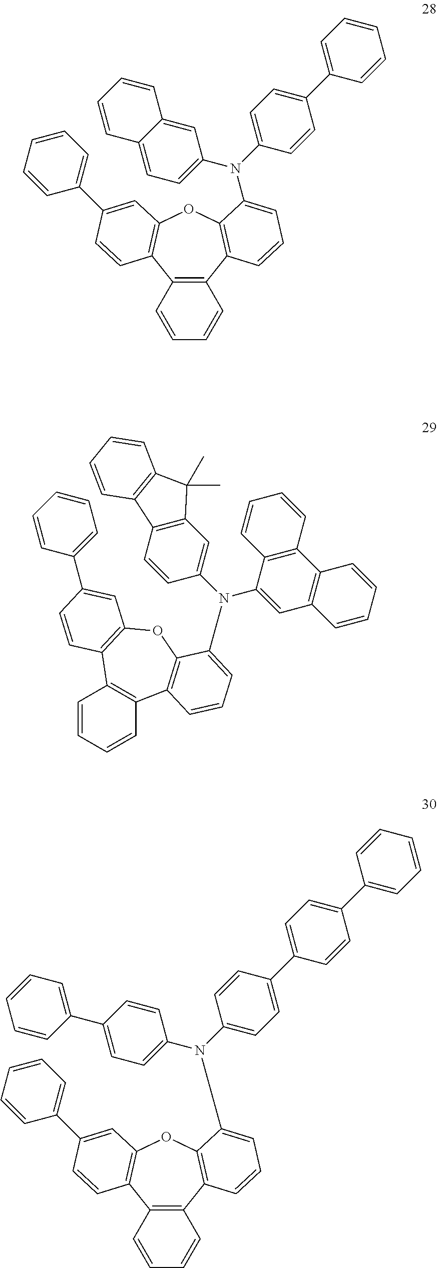 [SMILES: c1ccc(-c2ccc(N(c3ccc4ccccc4c3)c3cccc4c3Oc3cc(-c5ccccc5)ccc3-c3ccccc3-4)cc2)cc1 ; CC1(C)c2ccccc2-c2ccc(N(c3cccc4c3Oc3cc(-c5ccccc5)ccc3-c3ccccc3-4)c3cc4ccccc4c4ccccc34)cc21 ; c1ccc(-c2ccc(-c3ccc(N(c4ccc(-c5ccccc5)cc4)c4cccc5c4Oc4cc(-c6ccccc6)ccc4-c4ccccc4-5)cc3)cc2)cc1]

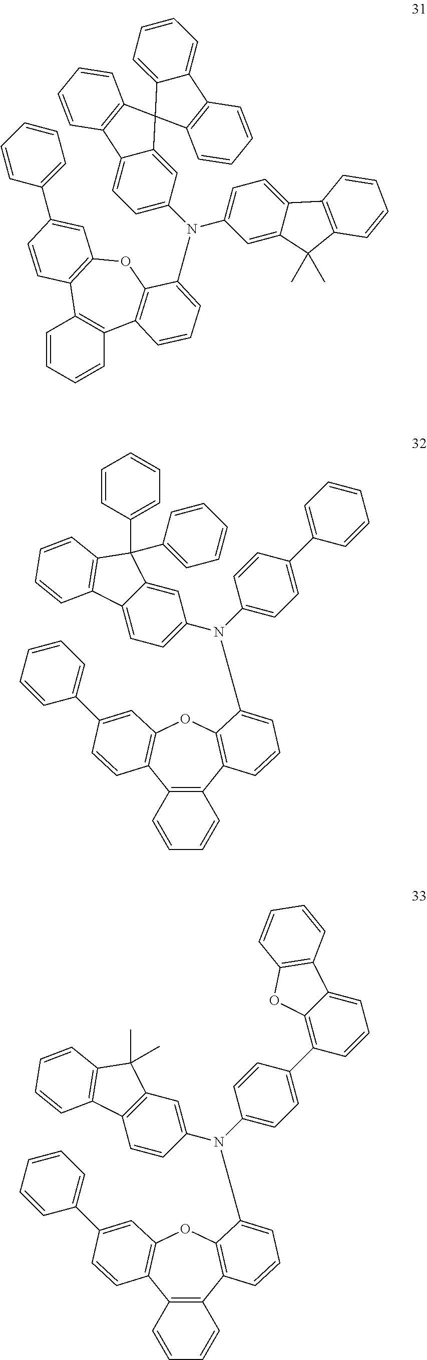 [SMILES: CC1(C)c2ccccc2-c2ccc(N(c3ccc4c(c3)C3(c5ccccc5-c5ccccc53)c3ccccc3-4)c3cccc4c3Oc3cc(-c5ccccc5)ccc3-c3ccccc3-4)cc21 ; c1ccc(-c2ccc(N(c3ccc4c(c3)C(c3ccccc3)(c3ccccc3)c3ccccc3-4)c3cccc4c3Oc3cc(-c5ccccc5)ccc3-c3ccccc3-4)cc2)cc1 ; CC1(C)c2ccccc2-c2ccc(N(c3ccc(-c4cccc5c4oc4ccccc45)cc3)c3cccc4c3Oc3cc(-c5ccccc5)ccc3-c3ccccc3-4)cc21]

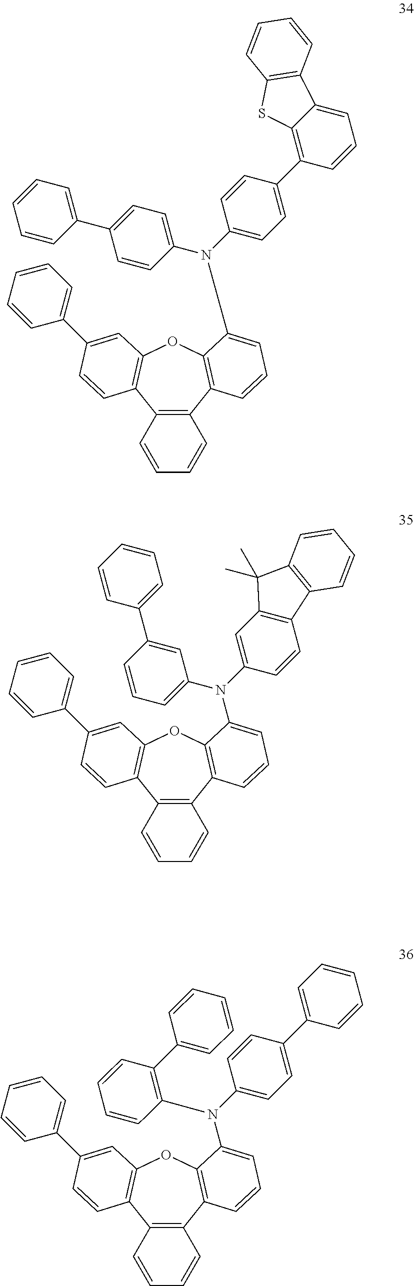 [SMILES: c1ccc(-c2ccc(N(c3ccc(-c4cccc5c4sc4ccccc45)cc3)c3cccc4c3Oc3cc(-c5ccccc5)ccc3-c3ccccc3-4)cc2)cc1 ; CC1(C)c2ccccc2-c2ccc(N(c3cccc(-c4ccccc4)c3)c3cccc4c3Oc3cc(-c5ccccc5)ccc3-c3ccccc3-4)cc21 ; c1ccc(-c2ccc(N(c3ccccc3-c3ccccc3)c3cccc4c3Oc3cc(-c5ccccc5)ccc3-c3ccccc3-4)cc2)cc1]

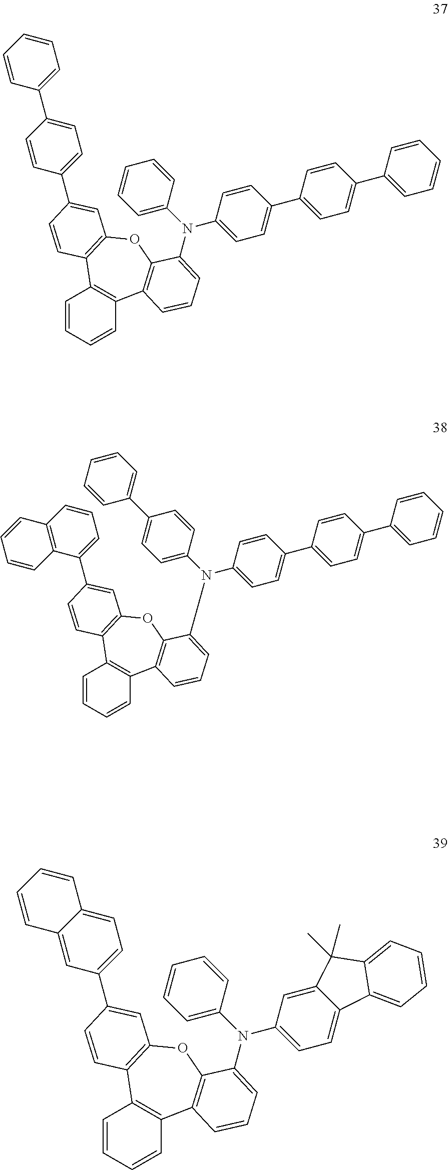 [SMILES: c1ccc(-c2ccc(-c3ccc(N(c4ccccc4)c4cccc5c4Oc4cc(-c6ccc(-c7ccccc7)cc6)ccc4-c4ccccc4-5)cc3)cc2)cc1 ; c1ccc(-c2ccc(-c3ccc(N(c4ccc(-c5ccccc5)cc4)c4cccc5c4Oc4cc(-c6cccc7ccccc67)ccc4-c4ccccc4-5)cc3)cc2)cc1 ; CC1(C)c2ccccc2-c2ccc(N(c3ccccc3)c3cccc4c3Oc3cc(-c5ccc6ccccc6c5)ccc3-c3ccccc3-4)cc21]

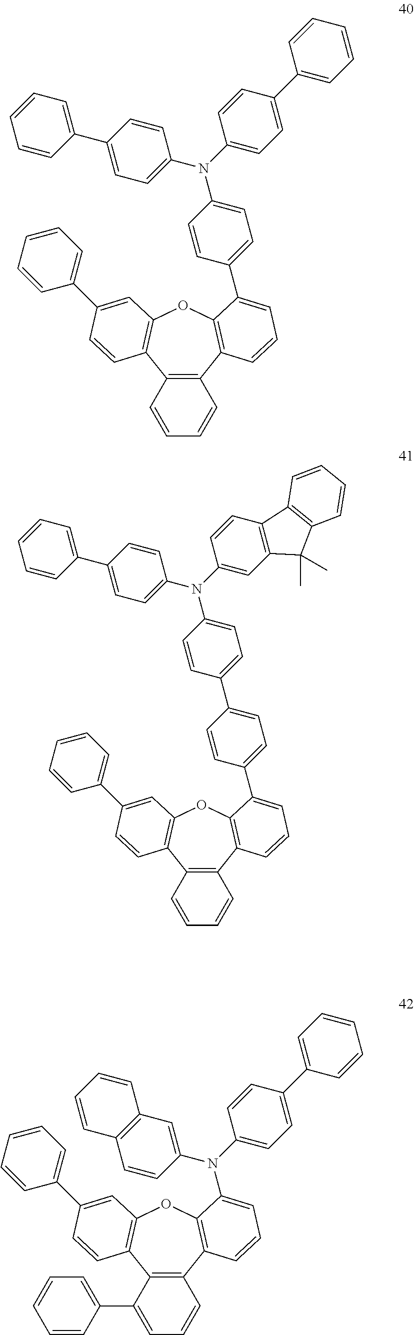 [SMILES: c1ccc(-c2ccc(N(c3ccc(-c4ccccc4)cc3)c3ccc(-c4cccc5c4Oc4cc(-c6ccccc6)ccc4-c4ccccc4-5)cc3)cc2)cc1 ; CC1(C)c2ccccc2-c2ccc(N(c3ccc(-c4ccccc4)cc3)c3ccc(-c4ccc(-c5cccc6c5Oc5cc(-c7ccccc7)ccc5-c5ccccc5-6)cc4)cc3)cc21 ; c1ccc(-c2ccc(N(c3ccc4ccccc4c3)c3cccc4c3Oc3cc(-c5ccccc5)ccc3-c3c(-c5ccccc5)cccc3-4)cc2)cc1]

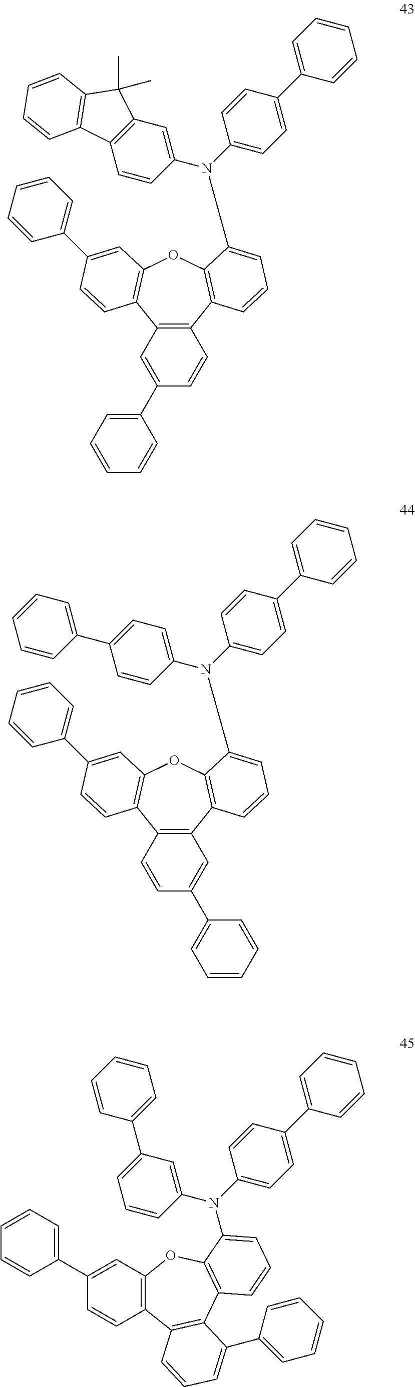 [SMILES: CC1(C)c2ccccc2-c2ccc(N(c3ccc(-c4ccccc4)cc3)c3cccc4c3Oc3cc(-c5ccccc5)ccc3-c3cc(-c5ccccc5)ccc3-4)cc21 ; c1ccc(-c2ccc(N(c3ccc(-c4ccccc4)cc3)c3cccc4c3Oc3cc(-c5ccccc5)ccc3-c3ccc(-c5ccccc5)cc3-4)cc2)cc1 ; c1ccc(-c2ccc(N(c3cccc(-c4ccccc4)c3)c3cccc4c3Oc3cc(-c5ccccc5)ccc3-c3cccc(-c5ccccc5)c3-4)cc2)cc1]

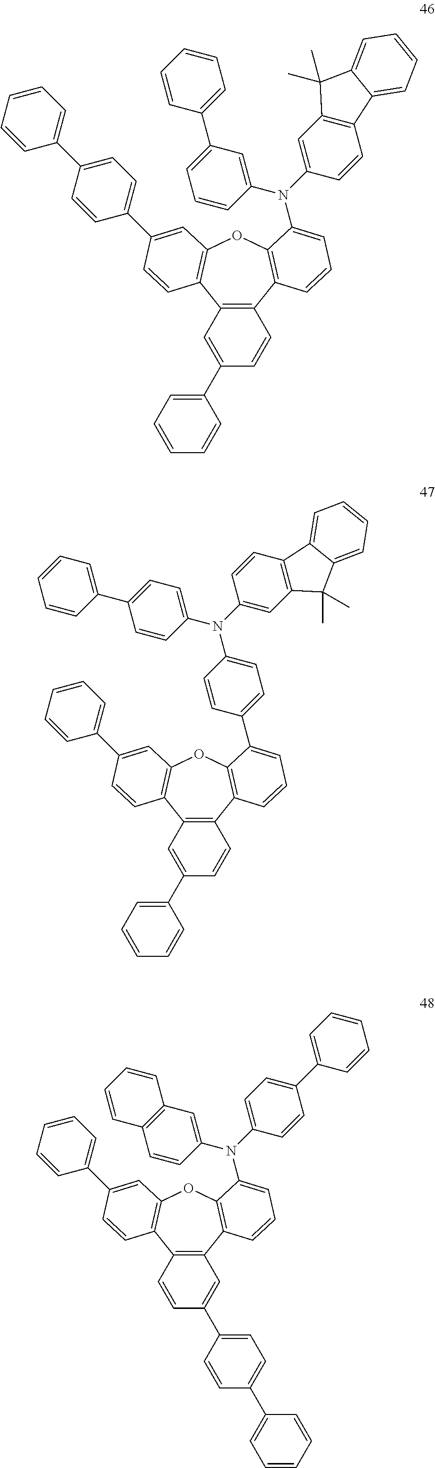 [SMILES: CC1(C)c2ccccc2-c2ccc(N(c3cccc(-c4ccccc4)c3)c3cccc4c3Oc3cc(-c5ccc(-c6ccccc6)cc5)ccc3-c3cc(-c5ccccc5)ccc3-4)cc21 ; CC1(C)c2ccccc2-c2ccc(N(c3ccc(-c4ccccc4)cc3)c3ccc(-c4cccc5c4Oc4cc(-c6ccccc6)ccc4-c4cc(-c6ccccc6)ccc4-5)cc3)cc21 ; c1ccc(-c2ccc(-c3ccc4c(c3)-c3cccc(N(c5ccc(-c6ccccc6)cc5)c5ccc6ccccc6c5)c3Oc3cc(-c5ccccc5)ccc3-4)cc2)cc1]

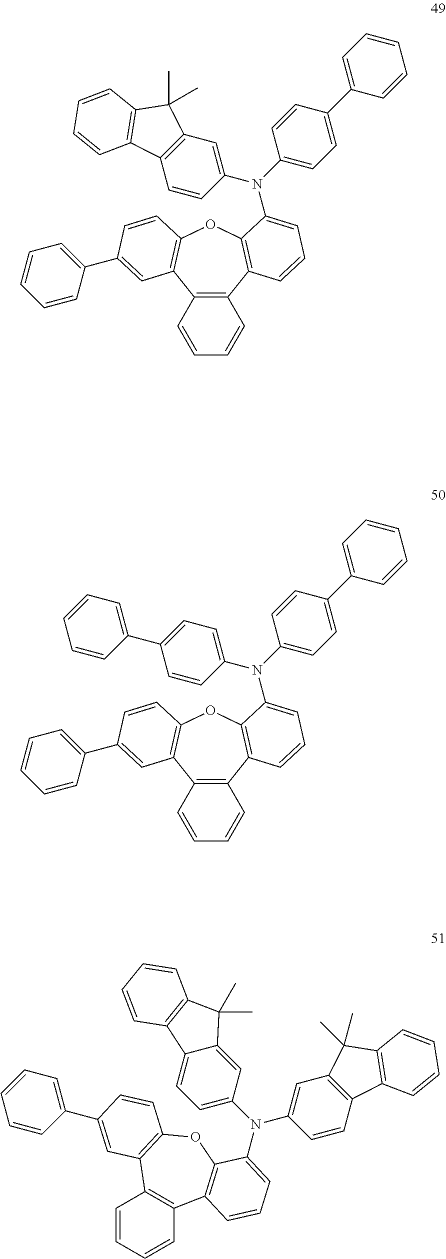 [SMILES: CC1(C)c2ccccc2-c2ccc(N(c3ccc(-c4ccccc4)cc3)c3cccc4c3Oc3ccc(-c5ccccc5)cc3-c3ccccc3-4)cc21 ; c1ccc(-c2ccc(N(c3ccc(-c4ccccc4)cc3)c3cccc4c3Oc3ccc(-c5ccccc5)cc3-c3ccccc3-4)cc2)cc1 ; CC1(C)c2ccccc2-c2ccc(N(c3ccc4c(c3)C(C)(C)c3ccccc3-4)c3cccc4c3Oc3ccc(-c5ccccc5)cc3-c3ccccc3-4)cc21]

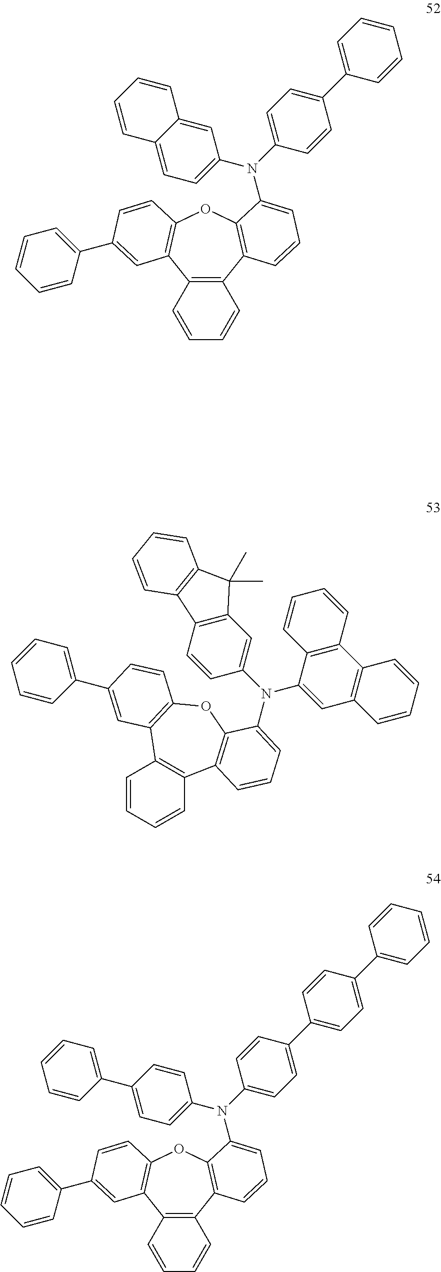 [SMILES: c1ccc(-c2ccc(N(c3ccc4ccccc4c3)c3cccc4c3Oc3ccc(-c5ccccc5)cc3-c3ccccc3-4)cc2)cc1 ; CC1(C)c2ccccc2-c2ccc(N(c3cccc4c3Oc3ccc(-c5ccccc5)cc3-c3ccccc3-4)c3cc4ccccc4c4ccccc34)cc21 ; c1ccc(-c2ccc(-c3ccc(N(c4ccc(-c5ccccc5)cc4)c4cccc5c4Oc4ccc(-c6ccccc6)cc4-c4ccccc4-5)cc3)cc2)cc1]

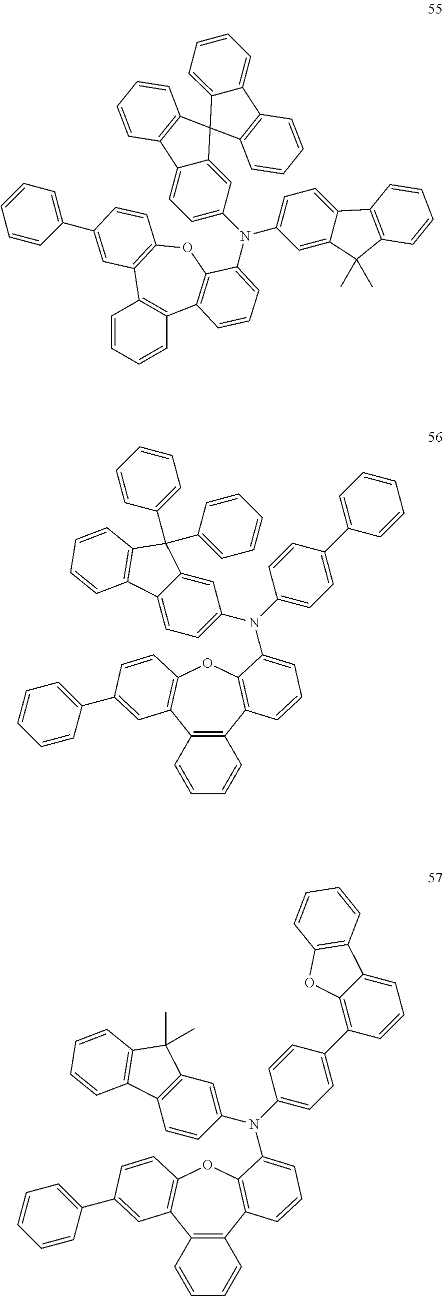 [SMILES: CC1(C)c2ccccc2-c2ccc(N(c3ccc4c(c3)C3(c5ccccc5-c5ccccc53)c3ccccc3-4)c3cccc4c3Oc3ccc(-c5ccccc5)cc3-c3ccccc3-4)cc21 ; c1ccc(-c2ccc(N(c3ccc4c(c3)C(c3ccccc3)(c3ccccc3)c3ccccc3-4)c3cccc4c3Oc3ccc(-c5ccccc5)cc3-c3ccccc3-4)cc2)cc1 ; CC1(C)c2ccccc2-c2ccc(N(c3ccc(-c4cccc5c4oc4ccccc45)cc3)c3cccc4c3Oc3ccc(-c5ccccc5)cc3-c3ccccc3-4)cc21]

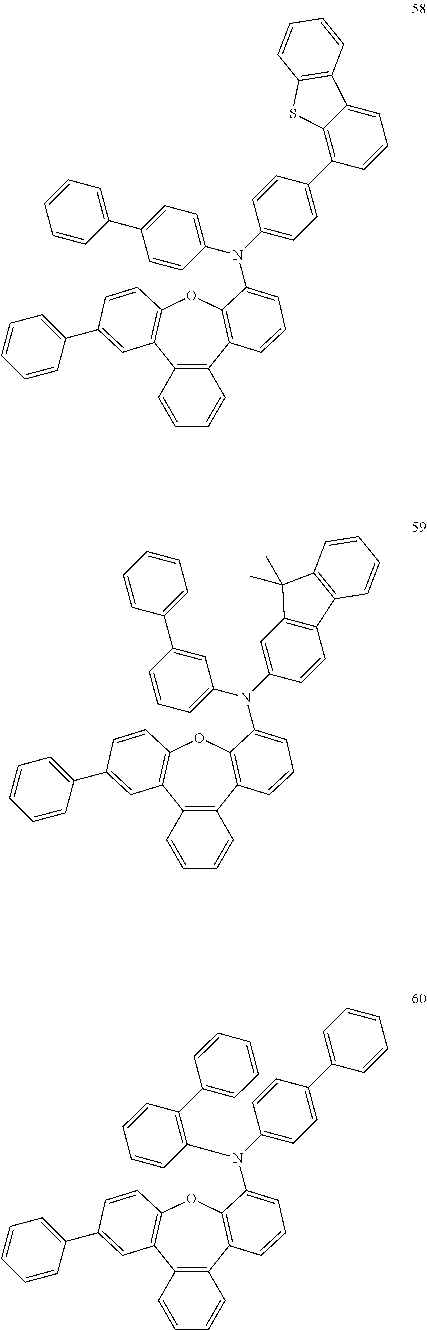 [SMILES: c1ccc(-c2ccc(N(c3ccc(-c4cccc5c4sc4ccccc45)cc3)c3cccc4c3Oc3ccc(-c5ccccc5)cc3-c3ccccc3-4)cc2)cc1 ; CC1(C)c2ccccc2-c2ccc(N(c3cccc(-c4ccccc4)c3)c3cccc4c3Oc3ccc(-c5ccccc5)cc3-c3ccccc3-4)cc21 ; c1ccc(-c2ccc(N(c3ccccc3-c3ccccc3)c3cccc4c3Oc3ccc(-c5ccccc5)cc3-c3ccccc3-4)cc2)cc1]

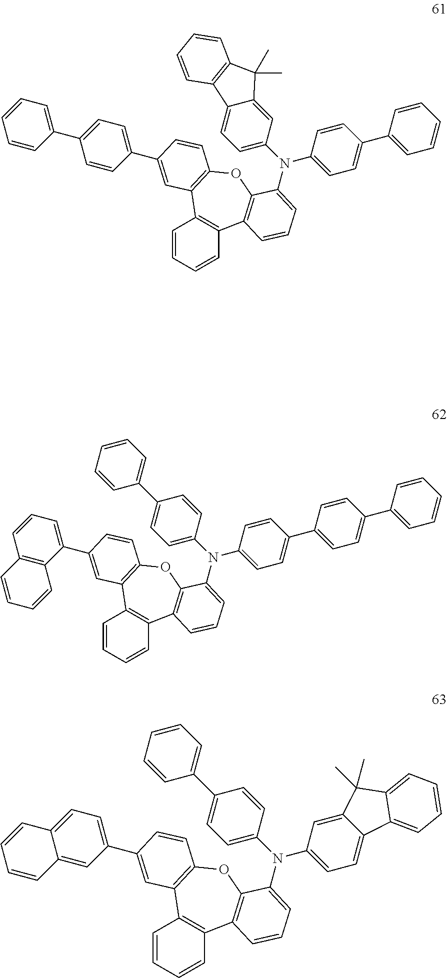 [SMILES: CC1(C)c2ccccc2-c2ccc(N(c3ccc(-c4ccccc4)cc3)c3cccc4c3Oc3ccc(-c5ccc(-c6ccccc6)cc5)cc3-c3ccccc3-4)cc21 ; c1ccc(-c2ccc(-c3ccc(N(c4ccc(-c5ccccc5)cc4)c4cccc5c4Oc4ccc(-c6cccc7ccccc67)cc4-c4ccccc4-5)cc3)cc2)cc1 ; CC1(C)c2ccccc2-c2ccc(N(c3ccc(-c4ccccc4)cc3)c3cccc4c3Oc3ccc(-c5ccc6ccccc6c5)cc3-c3ccccc3-4)cc21]

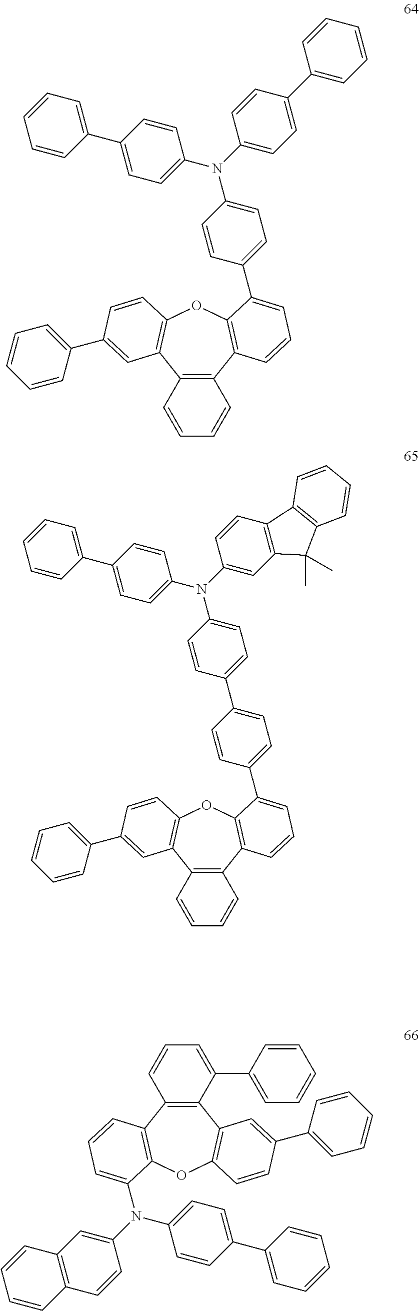 [SMILES: c1ccc(-c2ccc(N(c3ccc(-c4ccccc4)cc3)c3ccc(-c4cccc5c4Oc4ccc(-c6ccccc6)cc4-c4ccccc4-5)cc3)cc2)cc1 ; CC1(C)c2ccccc2-c2ccc(N(c3ccc(-c4ccccc4)cc3)c3ccc(-c4ccc(-c5cccc6c5Oc5ccc(-c7ccccc7)cc5-c5ccccc5-6)cc4)cc3)cc21 ; c1ccc(-c2ccc(N(c3ccc4ccccc4c3)c3cccc4c3Oc3ccc(-c5ccccc5)cc3-c3c(-c5ccccc5)cccc3-4)cc2)cc1]

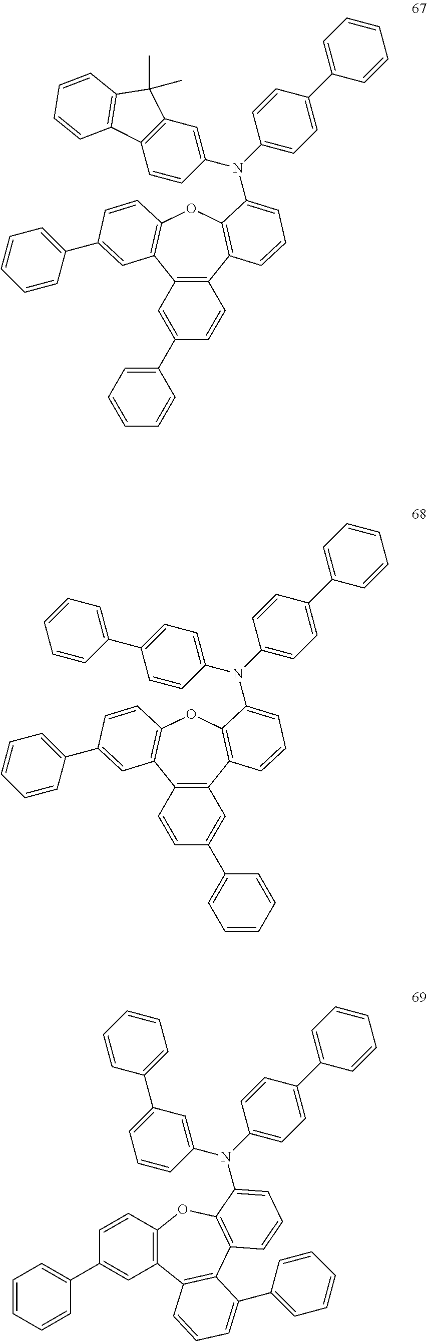 [SMILES: CC1(C)c2ccccc2-c2ccc(N(c3ccc(-c4ccccc4)cc3)c3cccc4c3Oc3ccc(-c5ccccc5)cc3-c3cc(-c5ccccc5)ccc3-4)cc21 ; c1ccc(-c2ccc(N(c3ccc(-c4ccccc4)cc3)c3cccc4c3Oc3ccc(-c5ccccc5)cc3-c3ccc(-c5ccccc5)cc3-4)cc2)cc1 ; c1ccc(-c2ccc(N(c3cccc(-c4ccccc4)c3)c3cccc4c3Oc3ccc(-c5ccccc5)cc3-c3cccc(-c5ccccc5)c3-4)cc2)cc1]

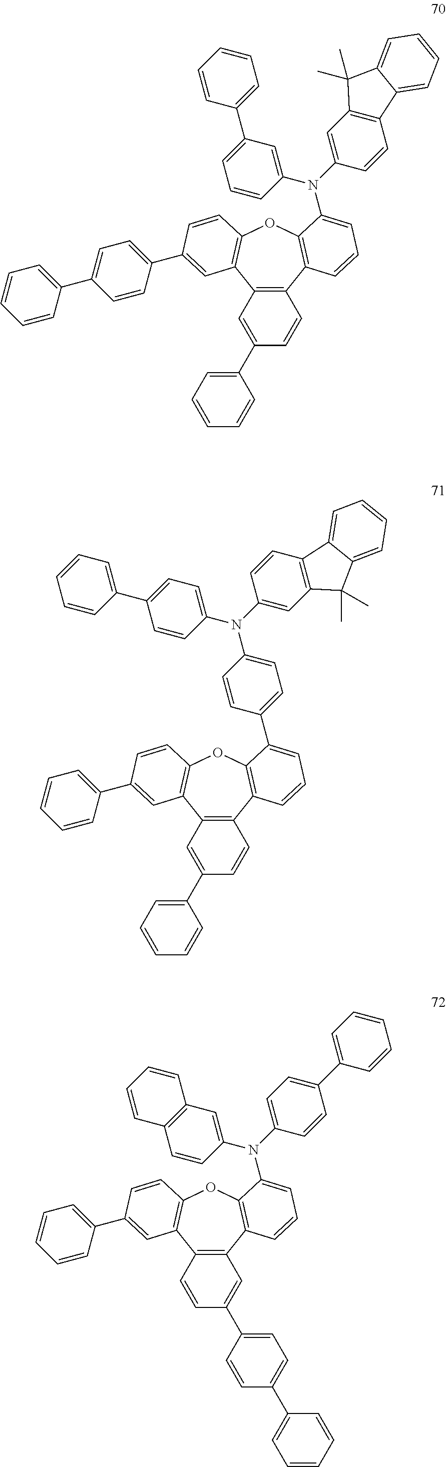 [SMILES: CC1(C)c2ccccc2-c2ccc(N(c3cccc(-c4ccccc4)c3)c3cccc4c3Oc3ccc(-c5ccc(-c6ccccc6)cc5)cc3-c3cc(-c5ccccc5)ccc3-4)cc21 ; CC1(C)c2ccccc2-c2ccc(N(c3ccc(-c4ccccc4)cc3)c3ccc(-c4cccc5c4Oc4ccc(-c6ccccc6)cc4-c4cc(-c6ccccc6)ccc4-5)cc3)cc21 ; c1ccc(-c2ccc(-c3ccc4c(c3)-c3cccc(N(c5ccc(-c6ccccc6)cc5)c5ccc6ccccc6c5)c3Oc3ccc(-c5ccccc5)cc3-4)cc2)cc1]

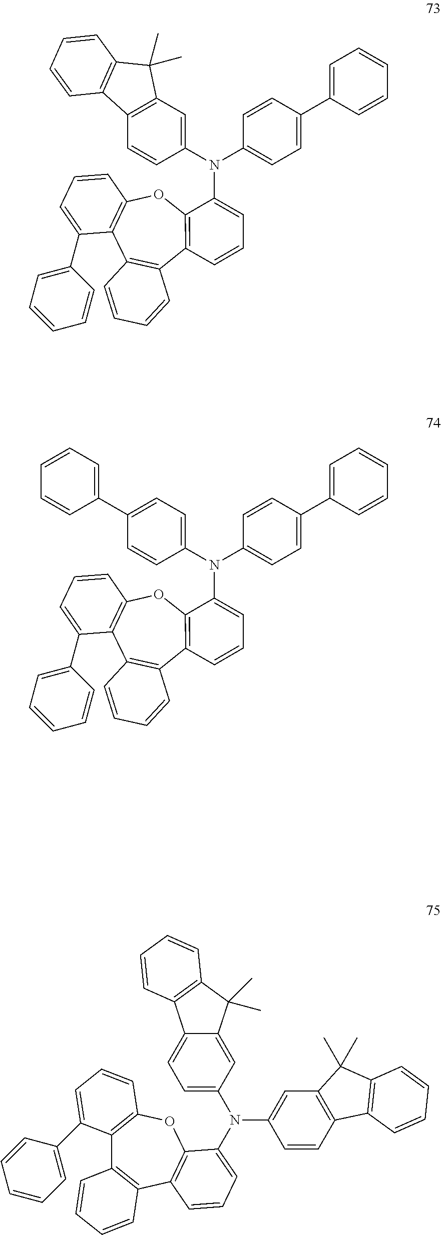 [SMILES: CC1(C)c2ccccc2-c2ccc(N(c3ccc(-c4ccccc4)cc3)c3cccc4c3Oc3cccc(-c5ccccc5)c3-c3ccccc3-4)cc21 ; c1ccc(-c2ccc(N(c3ccc(-c4ccccc4)cc3)c3cccc4c3Oc3cccc(-c5ccccc5)c3-c3ccccc3-4)cc2)cc1 ; CC1(C)c2ccccc2-c2ccc(N(c3ccc4c(c3)C(C)(C)c3ccccc3-4)c3cccc4c3Oc3cccc(-c5ccccc5)c3-c3ccccc3-4)cc21]

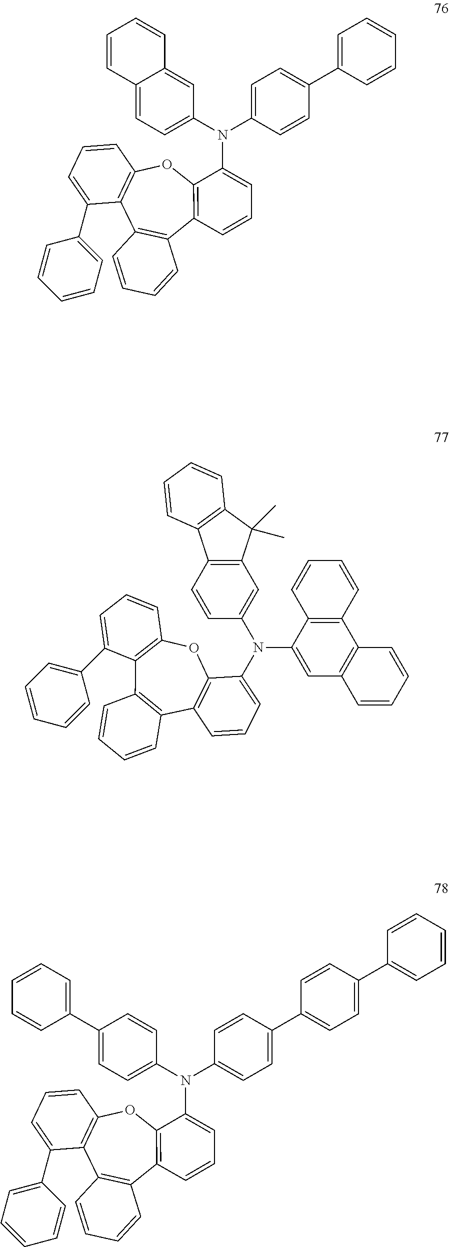 [SMILES: c1ccc(-c2ccc(N(c3ccc4ccccc4c3)c3cccc4c3Oc3cccc(-c5ccccc5)c3-c3ccccc3-4)cc2)cc1 ; CC1(C)c2ccccc2-c2ccc(N(c3cccc4c3Oc3cccc(-c5ccccc5)c3-c3ccccc3-4)c3cc4ccccc4c4ccccc34)cc21 ; c1ccc(-c2ccc(-c3ccc(N(c4ccc(-c5ccccc5)cc4)c4cccc5c4Oc4cccc(-c6ccccc6)c4-c4ccccc4-5)cc3)cc2)cc1]

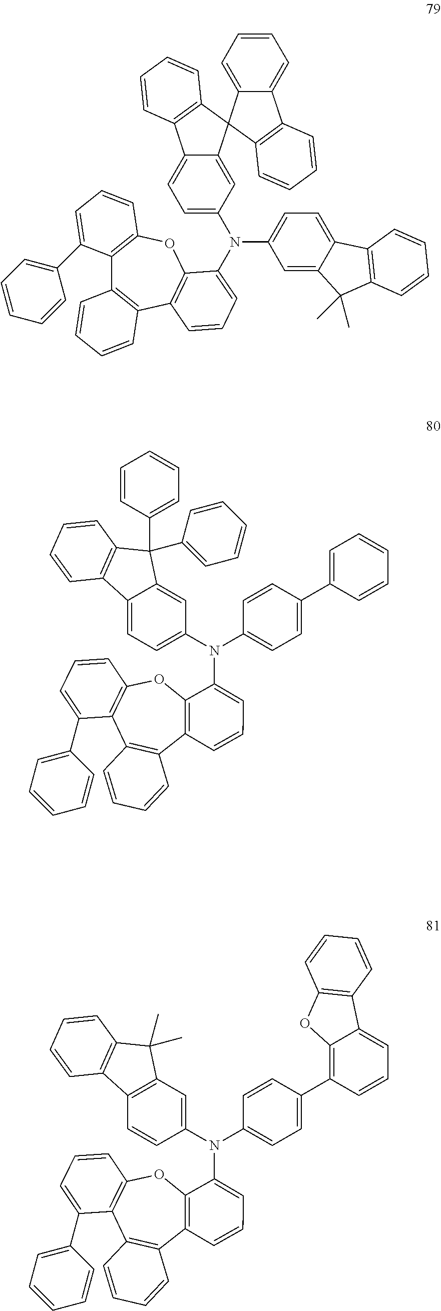 [SMILES: CC1(C)c2ccccc2-c2ccc(N(c3ccc4c(c3)C3(c5ccccc5-c5ccccc53)c3ccccc3-4)c3cccc4c3Oc3cccc(-c5ccccc5)c3-c3ccccc3-4)cc21 ; c1ccc(-c2ccc(N(c3ccc4c(c3)C(c3ccccc3)(c3ccccc3)c3ccccc3-4)c3cccc4c3Oc3cccc(-c5ccccc5)c3-c3ccccc3-4)cc2)cc1 ; CC1(C)c2ccccc2-c2ccc(N(c3ccc(-c4cccc5c4oc4ccccc45)cc3)c3cccc4c3Oc3cccc(-c5ccccc5)c3-c3ccccc3-4)cc21]

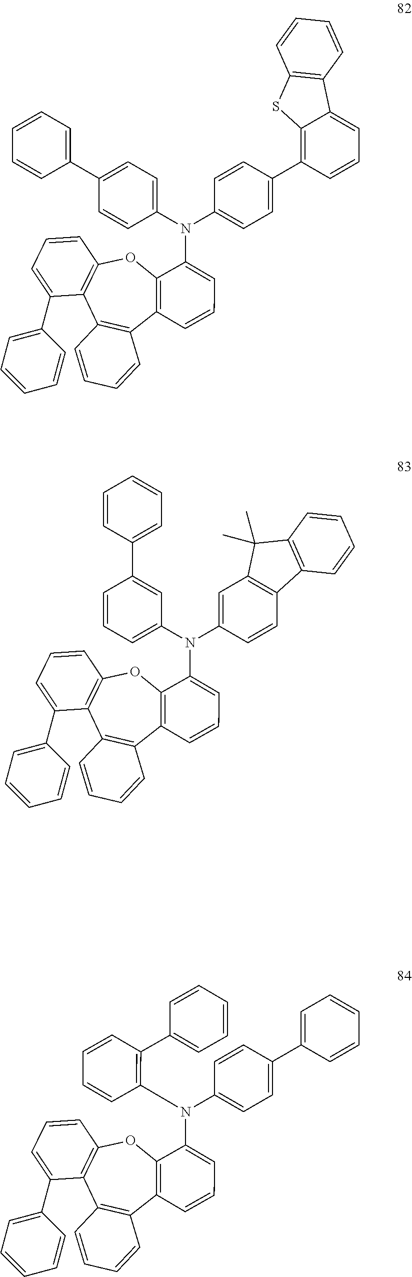 [SMILES: c1ccc(-c2ccc(N(c3ccc(-c4cccc5c4sc4ccccc45)cc3)c3cccc4c3Oc3cccc(-c5ccccc5)c3-c3ccccc3-4)cc2)cc1 ; CC1(C)c2ccccc2-c2ccc(N(c3cccc(-c4ccccc4)c3)c3cccc4c3Oc3cccc(-c5ccccc5)c3-c3ccccc3-4)cc21 ; c1ccc(-c2ccc(N(c3ccccc3-c3ccccc3)c3cccc4c3Oc3cccc(-c5ccccc5)c3-c3ccccc3-4)cc2)cc1]

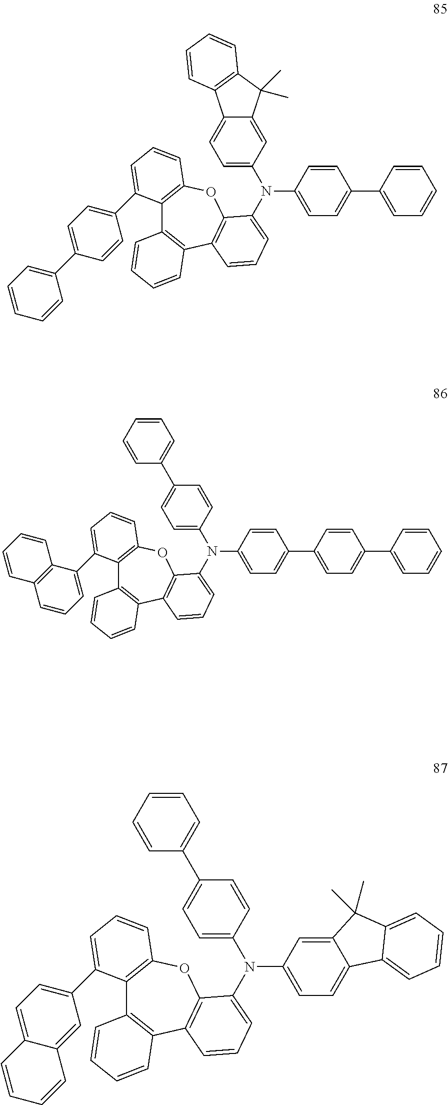 [SMILES: CC1(C)c2ccccc2-c2ccc(N(c3ccc(-c4ccccc4)cc3)c3cccc4c3Oc3cccc(-c5ccc(-c6ccccc6)cc5)c3-c3ccccc3-4)cc21 ; c1ccc(-c2ccc(-c3ccc(N(c4ccc(-c5ccccc5)cc4)c4cccc5c4Oc4cccc(-c6cccc7ccccc67)c4-c4ccccc4-5)cc3)cc2)cc1 ; CC1(C)c2ccccc2-c2ccc(N(c3ccc(-c4ccccc4)cc3)c3cccc4c3Oc3cccc(-c5ccc6ccccc6c5)c3-c3ccccc3-4)cc21]

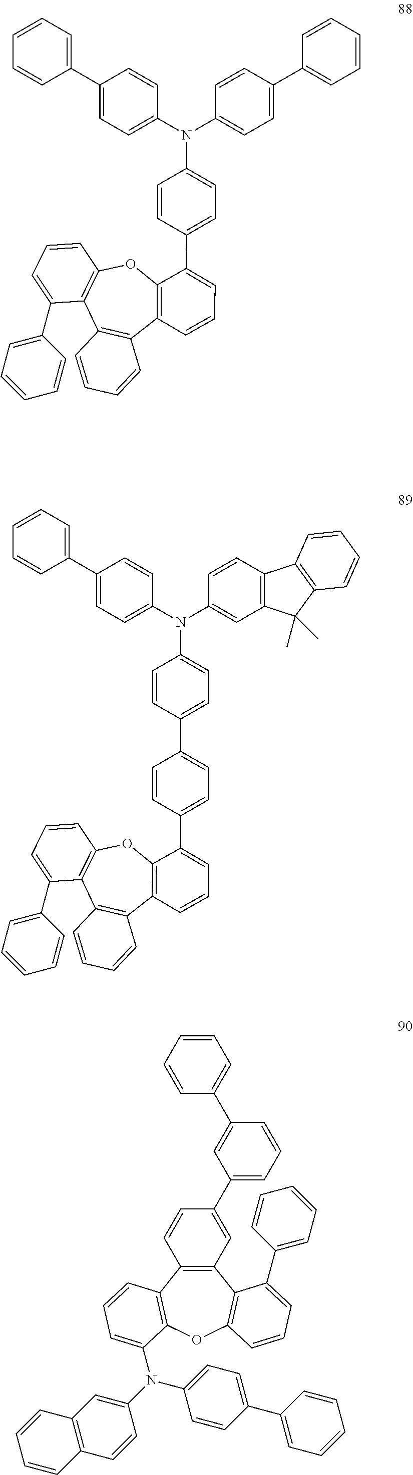 [SMILES: c1ccc(-c2ccc(N(c3ccc(-c4ccccc4)cc3)c3ccc(-c4cccc5c4Oc4cccc(-c6ccccc6)c4-c4ccccc4-5)cc3)cc2)cc1 ; CC1(C)c2ccccc2-c2ccc(N(c3ccc(-c4ccccc4)cc3)c3ccc(-c4ccc(-c5cccc6c5Oc5cccc(-c7ccccc7)c5-c5ccccc5-6)cc4)cc3)cc21 ; c1ccc(-c2ccc(N(c3ccc4ccccc4c3)c3cccc4c3Oc3cccc(-c5ccccc5)c3-c3cc(-c5cccc(-c6ccccc6)c5)ccc3-4)cc2)cc1]

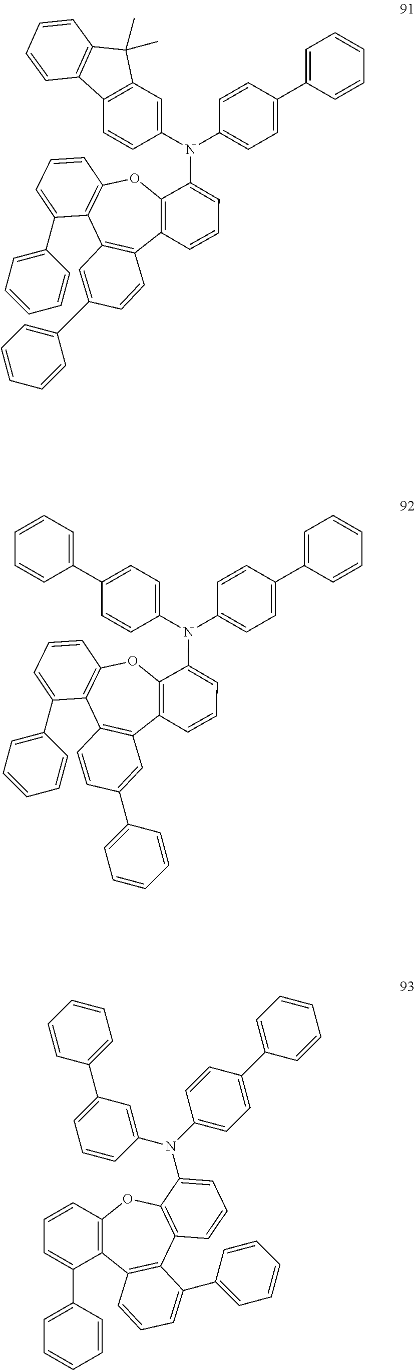 [SMILES: CC1(C)c2ccccc2-c2ccc(N(c3ccc(-c4ccccc4)cc3)c3cccc4c3Oc3cccc(-c5ccccc5)c3-c3cc(-c5ccccc5)ccc3-4)cc21 ; c1ccc(-c2ccc(N(c3ccc(-c4ccccc4)cc3)c3cccc4c3Oc3cccc(-c5ccccc5)c3-c3ccc(-c5ccccc5)cc3-4)cc2)cc1 ; c1ccc(-c2ccc(N(c3cccc(-c4ccccc4)c3)c3cccc4c3Oc3cccc(-c5ccccc5)c3-c3cccc(-c5ccccc5)c3-4)cc2)cc1]

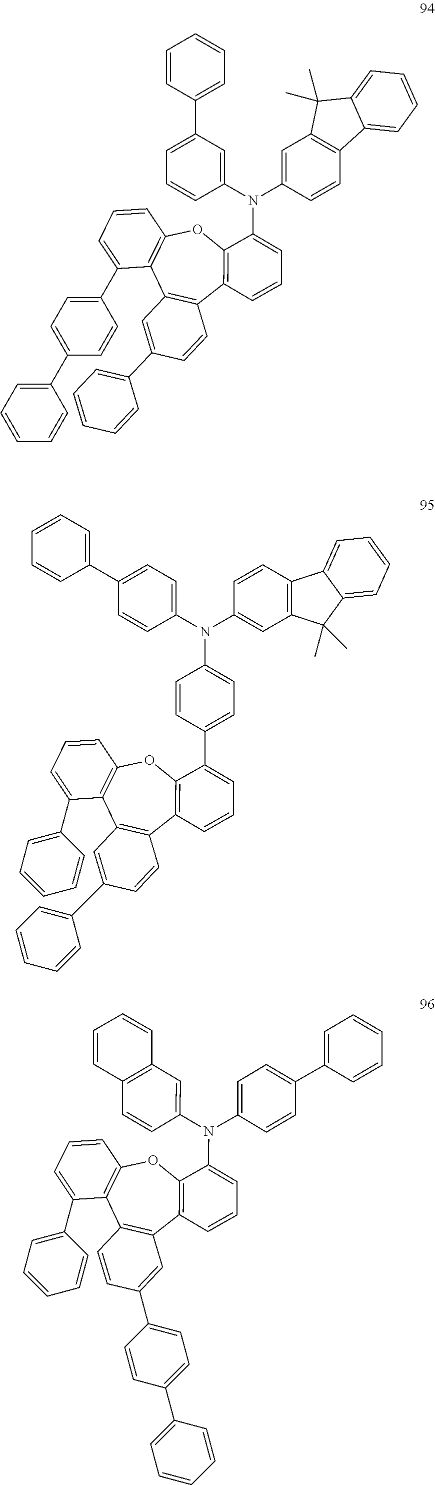 [SMILES: CC1(C)c2ccccc2-c2ccc(N(c3cccc(-c4ccccc4)c3)c3cccc4c3Oc3cccc(-c5ccc(-c6ccccc6)cc5)c3-c3cc(-c5ccccc5)ccc3-4)cc21 ; CC1(C)c2ccccc2-c2ccc(N(c3ccc(-c4ccccc4)cc3)c3ccc(-c4cccc5c4Oc4cccc(-c6ccccc6)c4-c4cc(-c6ccccc6)ccc4-5)cc3)cc21 ; c1ccc(-c2ccc(-c3ccc4c(c3)-c3cccc(N(c5ccc(-c6ccccc6)cc5)c5ccc6ccccc6c5)c3Oc3cccc(-c5ccccc5)c3-4)cc2)cc1]

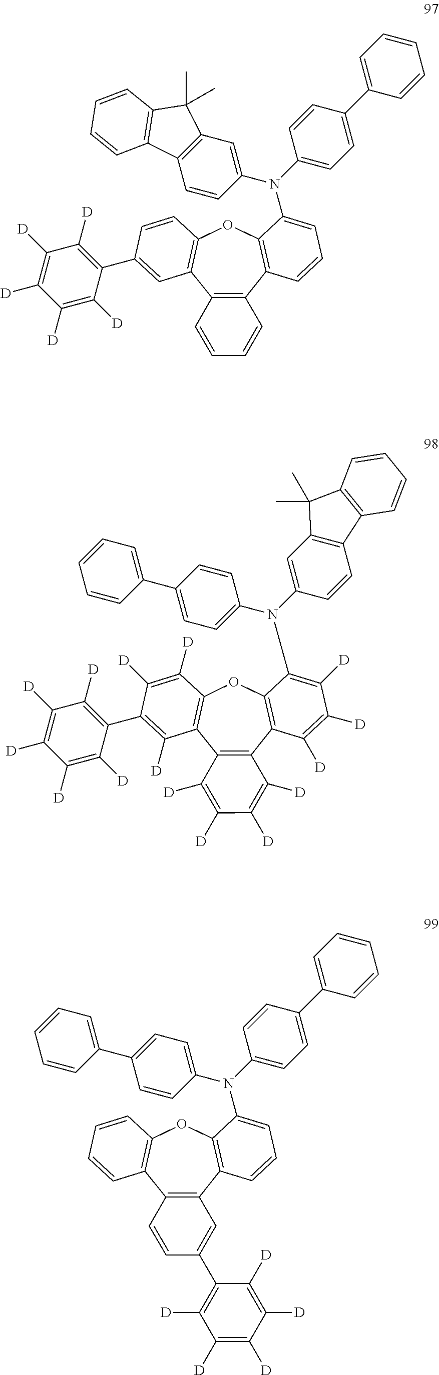 [SMILES: [2H]c1c([2H])c([2H])c(-c2ccc3c(c2)-c2ccccc2-c2cccc(N(c4ccc(-c5ccccc5)cc4)c4ccc5c(c4)C(C)(C)c4ccccc4-5)c2O3)c([2H])c1[2H] ; [2H]c1c([2H])c([2H])c(-c2c([2H])c([2H])c3c(c2[2H])-c2c([2H])c([2H])c([2H])c([2H])c2-c2c([2H])c([2H])c([2H])c(N(c4ccc(-c5ccccc5)cc4)c4ccc5c(c4)C(C)(C)c4ccccc4-5)c2O3)c([2H])c1[2H] ; [2H]c1c([2H])c([2H])c(-c2ccc3c(c2)-c2cccc(N(c4ccc(-c5ccccc5)cc4)c4ccc(-c5ccccc5)cc4)c2Oc2ccccc2-3)c([2H])c1[2H]]

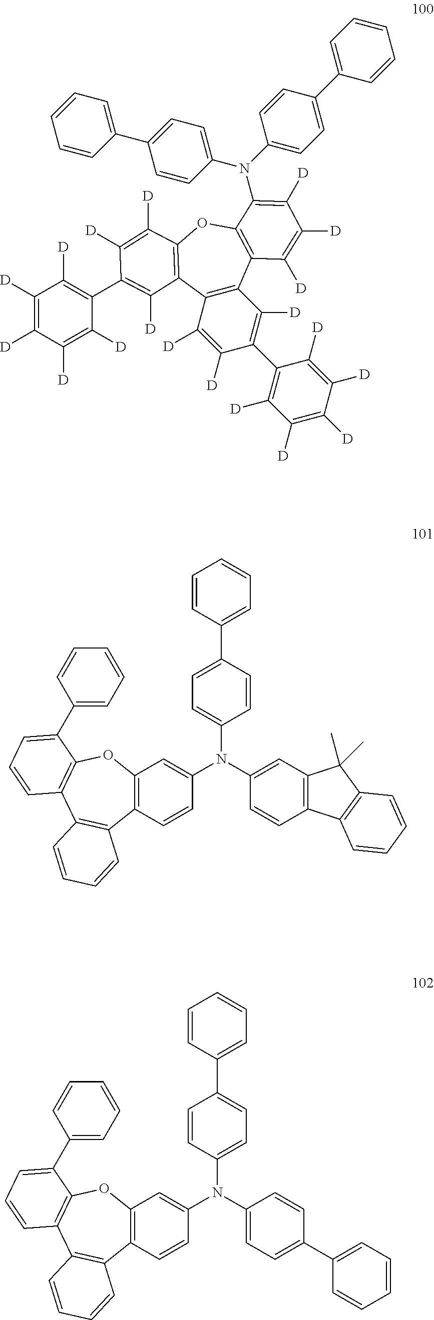 [SMILES: [2H]c1c([2H])c([2H])c(-c2c([2H])c([2H])c3c(c2[2H])-c2c([2H])c([2H])c(-c4c([2H])c([2H])c([2H])c([2H])c4[2H])c([2H])c2-c2c([2H])c([2H])c([2H])c(N(c4ccc(-c5ccccc5)cc4)c4ccc(-c5ccccc5)cc4)c2O3)c([2H])c1[2H] ; CC1(C)c2ccccc2-c2ccc(N(c3ccc(-c4ccccc4)cc3)c3ccc4c(c3)Oc3c(-c5ccccc5)cccc3-c3ccccc3-4)cc21 ; c1ccc(-c2ccc(N(c3ccc(-c4ccccc4)cc3)c3ccc4c(c3)Oc3c(-c5ccccc5)cccc3-c3ccccc3-4)cc2)cc1]

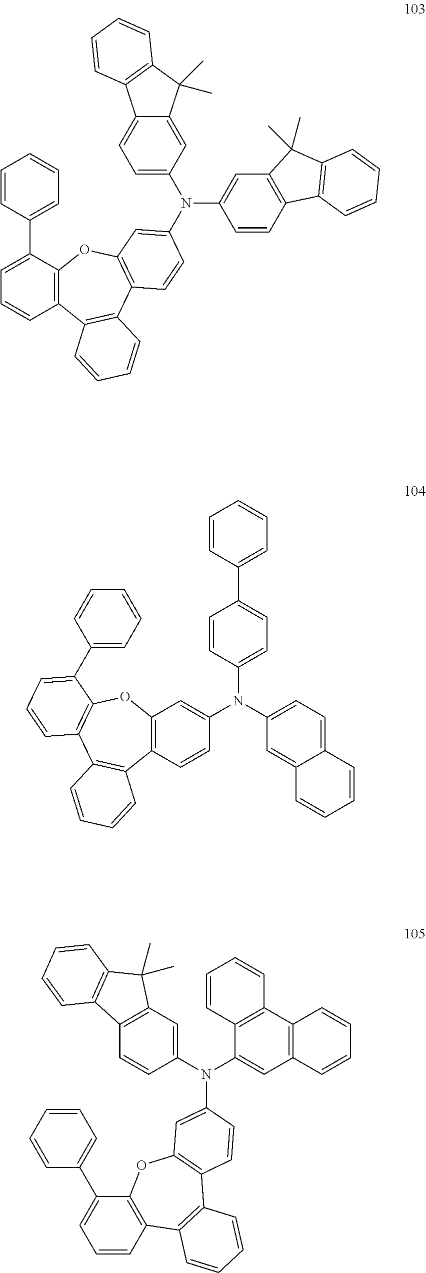 [SMILES: CC1(C)c2ccccc2-c2ccc(N(c3ccc4c(c3)Oc3c(-c5ccccc5)cccc3-c3ccccc3-4)c3ccc4c(c3)C(C)(C)c3ccccc3-4)cc21 ; c1ccc(-c2ccc(N(c3ccc4c(c3)Oc3c(-c5ccccc5)cccc3-c3ccccc3-4)c3ccc4ccccc4c3)cc2)cc1 ; CC1(C)c2ccccc2-c2ccc(N(c3ccc4c(c3)Oc3c(-c5ccccc5)cccc3-c3ccccc3-4)c3cc4ccccc4c4ccccc34)cc21]

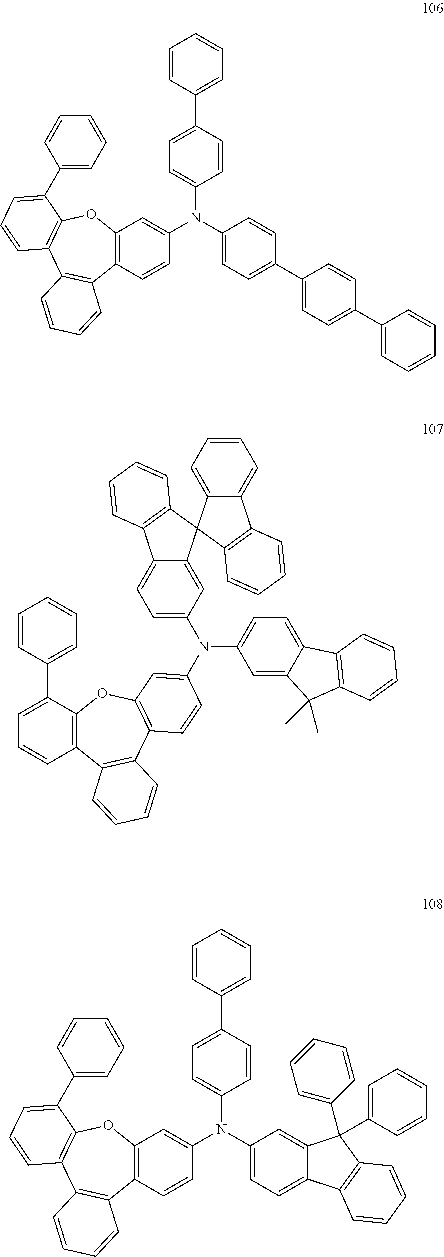 [SMILES: c1ccc(-c2ccc(-c3ccc(N(c4ccc(-c5ccccc5)cc4)c4ccc5c(c4)Oc4c(-c6ccccc6)cccc4-c4ccccc4-5)cc3)cc2)cc1 ; CC1(C)c2ccccc2-c2ccc(N(c3ccc4c(c3)Oc3c(-c5ccccc5)cccc3-c3ccccc3-4)c3ccc4c(c3)C3(c5ccccc5-c5ccccc53)c3ccccc3-4)cc21 ; c1ccc(-c2ccc(N(c3ccc4c(c3)Oc3c(-c5ccccc5)cccc3-c3ccccc3-4)c3ccc4c(c3)C(c3ccccc3)(c3ccccc3)c3ccccc3-4)cc2)cc1]

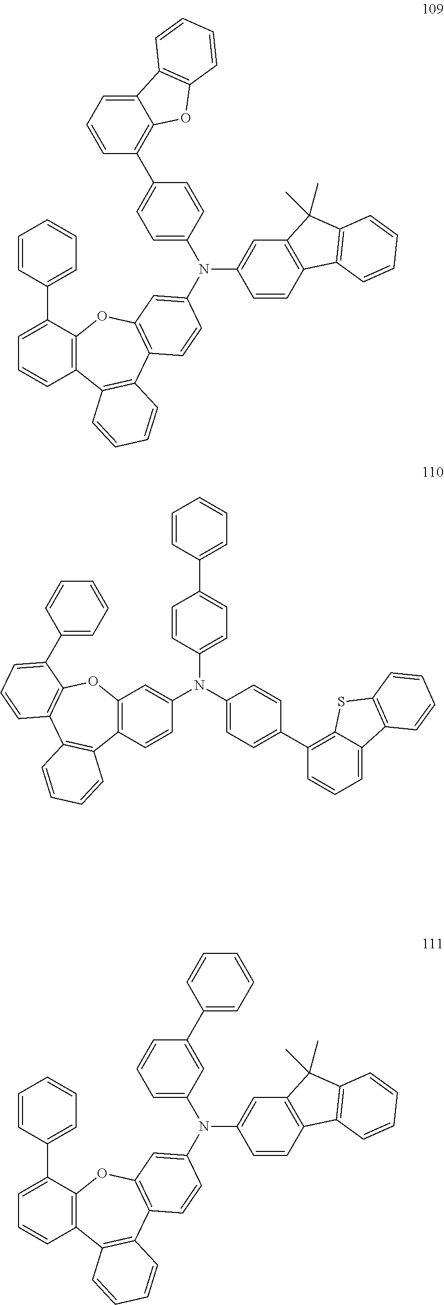 [SMILES: CC1(C)c2ccccc2-c2ccc(N(c3ccc(-c4cccc5c4oc4ccccc45)cc3)c3ccc4c(c3)Oc3c(-c5ccccc5)cccc3-c3ccccc3-4)cc21 ; c1ccc(-c2ccc(N(c3ccc(-c4cccc5c4sc4ccccc45)cc3)c3ccc4c(c3)Oc3c(-c5ccccc5)cccc3-c3ccccc3-4)cc2)cc1 ; CC1(C)c2ccccc2-c2ccc(N(c3cccc(-c4ccccc4)c3)c3ccc4c(c3)Oc3c(-c5ccccc5)cccc3-c3ccccc3-4)cc21]

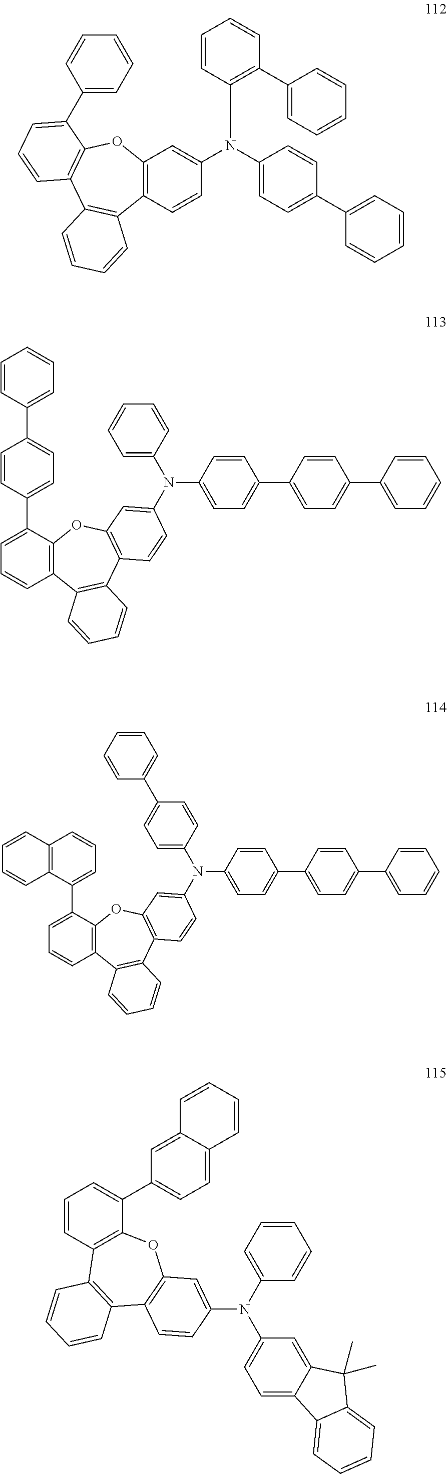 [SMILES: c1ccc(-c2ccc(N(c3ccc4c(c3)Oc3c(-c5ccccc5)cccc3-c3ccccc3-4)c3ccccc3-c3ccccc3)cc2)cc1 ; c1ccc(-c2ccc(-c3ccc(N(c4ccccc4)c4ccc5c(c4)Oc4c(-c6ccc(-c7ccccc7)cc6)cccc4-c4ccccc4-5)cc3)cc2)cc1 ; c1ccc(-c2ccc(-c3ccc(N(c4ccc(-c5ccccc5)cc4)c4ccc5c(c4)Oc4c(cccc4-c4cccc6ccccc46)-c4ccccc4-5)cc3)cc2)cc1 ; CC1(C)c2ccccc2-c2ccc(N(c3ccccc3)c3ccc4c(c3)Oc3c(-c5ccc6ccccc6c5)cccc3-c3ccccc3-4)cc21]

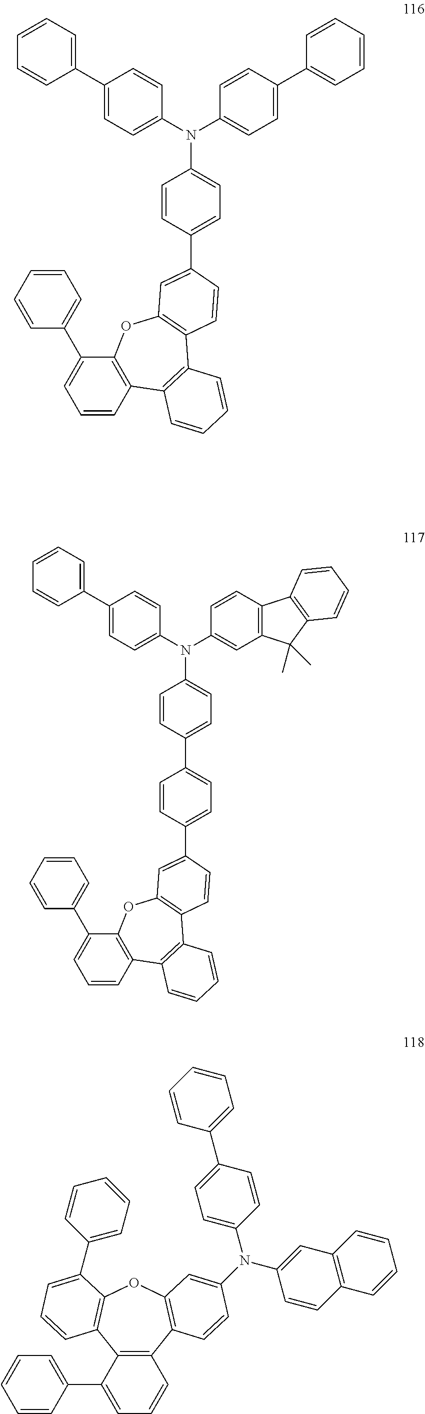 [SMILES: c1ccc(-c2ccc(N(c3ccc(-c4ccccc4)cc3)c3ccc(-c4ccc5c(c4)Oc4c(-c6ccccc6)cccc4-c4ccccc4-5)cc3)cc2)cc1 ; CC1(C)c2ccccc2-c2ccc(N(c3ccc(-c4ccccc4)cc3)c3ccc(-c4ccc(-c5ccc6c(c5)Oc5c(-c7ccccc7)cccc5-c5ccccc5-6)cc4)cc3)cc21 ; c1ccc(-c2ccc(N(c3ccc4c(c3)Oc3c(-c5ccccc5)cccc3-c3c(-c5ccccc5)cccc3-4)c3ccc4ccccc4c3)cc2)cc1]

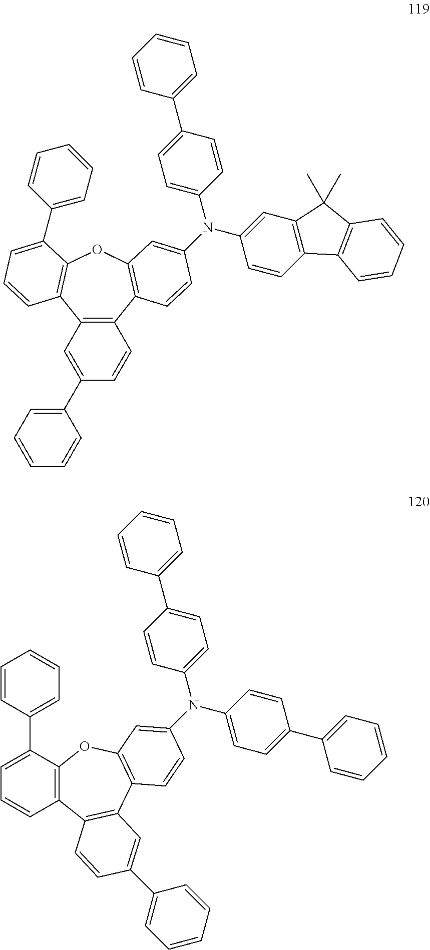 [SMILES: CC1(C)c2ccccc2-c2ccc(N(c3ccc(-c4ccccc4)cc3)c3ccc4c(c3)Oc3c(-c5ccccc5)cccc3-c3cc(-c5ccccc5)ccc3-4)cc21 ; c1ccc(-c2ccc(N(c3ccc(-c4ccccc4)cc3)c3ccc4c(c3)Oc3c(-c5ccccc5)cccc3-c3ccc(-c5ccccc5)cc3-4)cc2)cc1]

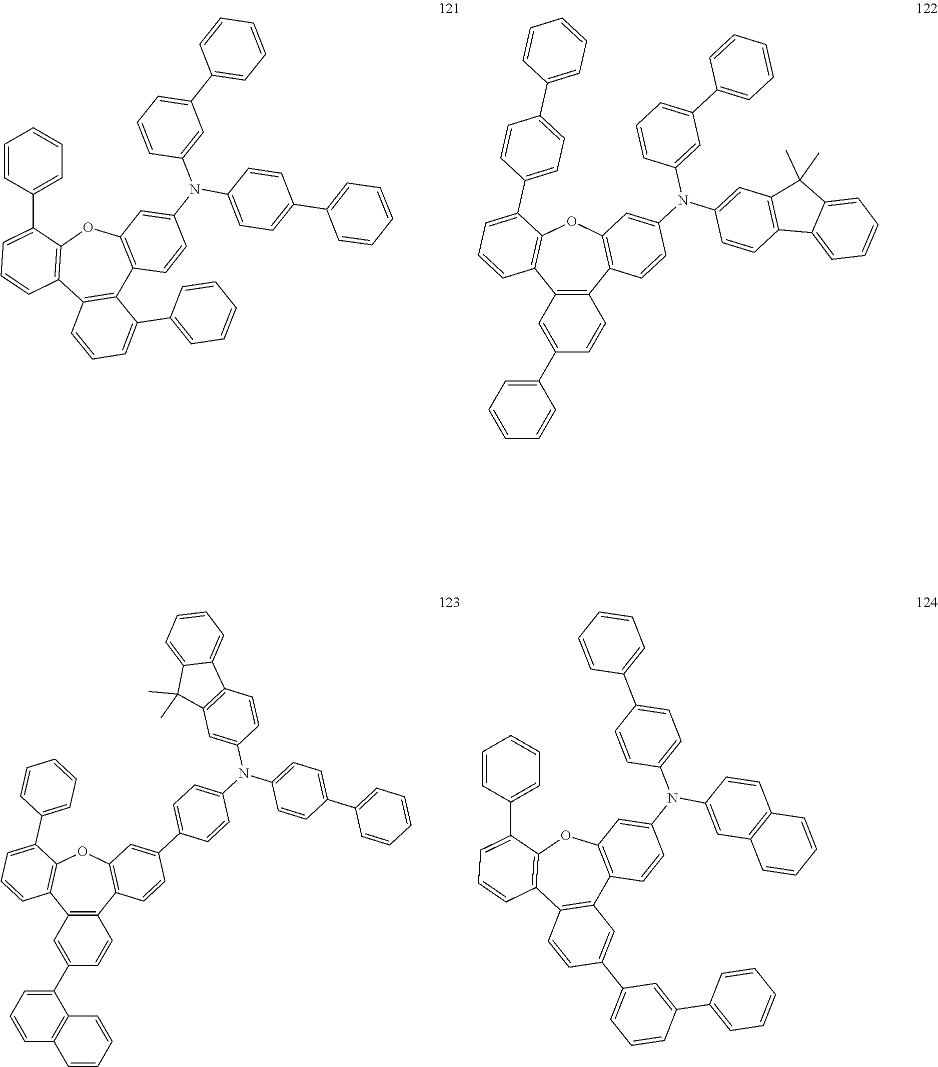 [SMILES: c1ccc(-c2ccc(N(c3cccc(-c4ccccc4)c3)c3ccc4c(c3)Oc3c(-c5ccccc5)cccc3-c3cccc(-c5ccccc5)c3-4)cc2)cc1 ; CC1(C)c2ccccc2-c2ccc(N(c3cccc(-c4ccccc4)c3)c3ccc4c(c3)Oc3c(-c5ccc(-c6ccccc6)cc5)cccc3-c3cc(-c5ccccc5)ccc3-4)cc21 ; CC1(C)c2ccccc2-c2ccc(N(c3ccc(-c4ccccc4)cc3)c3ccc(-c4ccc5c(c4)Oc4c(-c6ccccc6)cccc4-c4cc(-c6cccc7ccccc67)ccc4-5)cc3)cc21 ; c1ccc(-c2ccc(N(c3ccc4c(c3)Oc3c(-c5ccccc5)cccc3-c3ccc(-c5cccc(-c6ccccc6)c5)cc3-4)c3ccc4ccccc4c3)cc2)cc1]

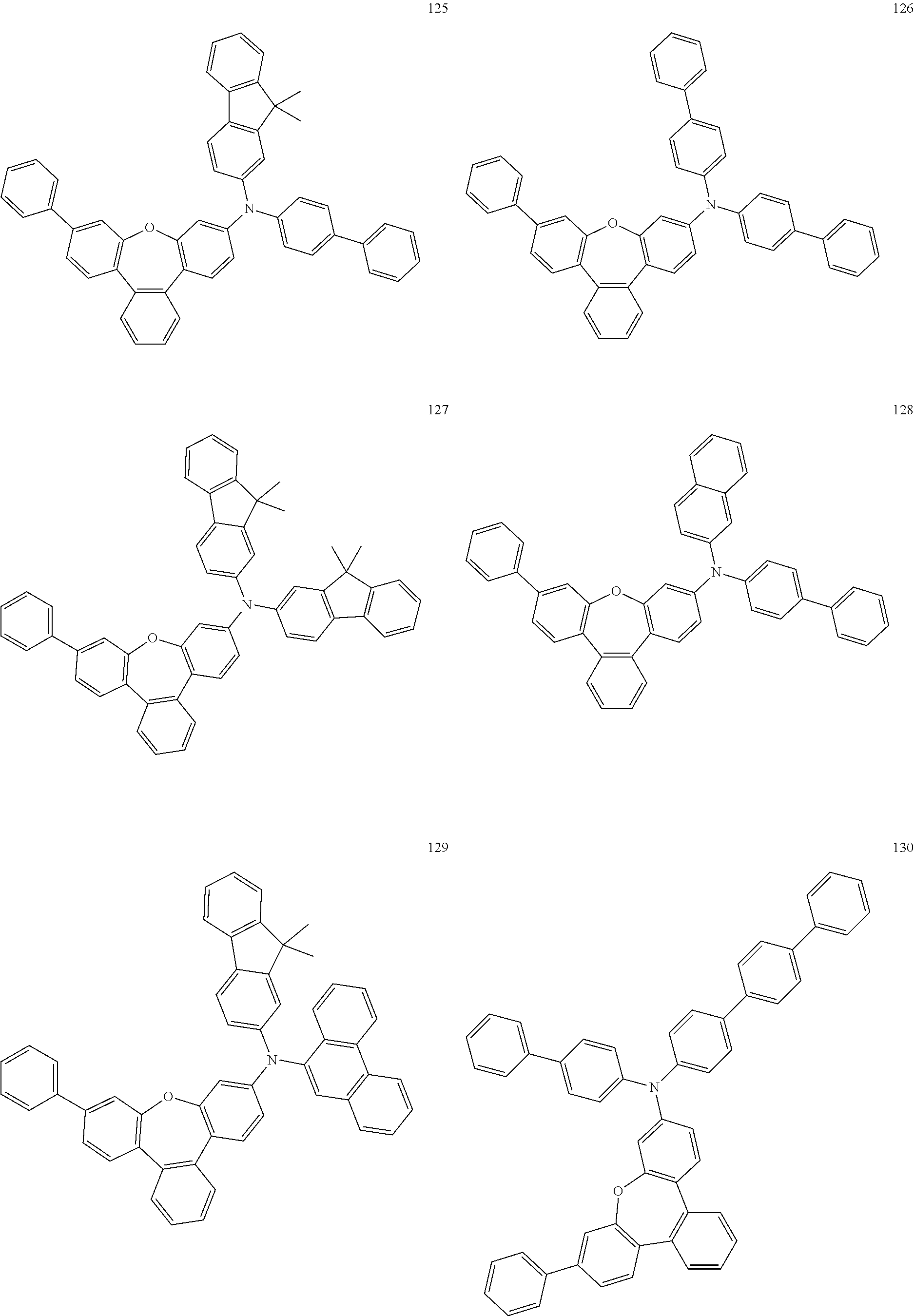

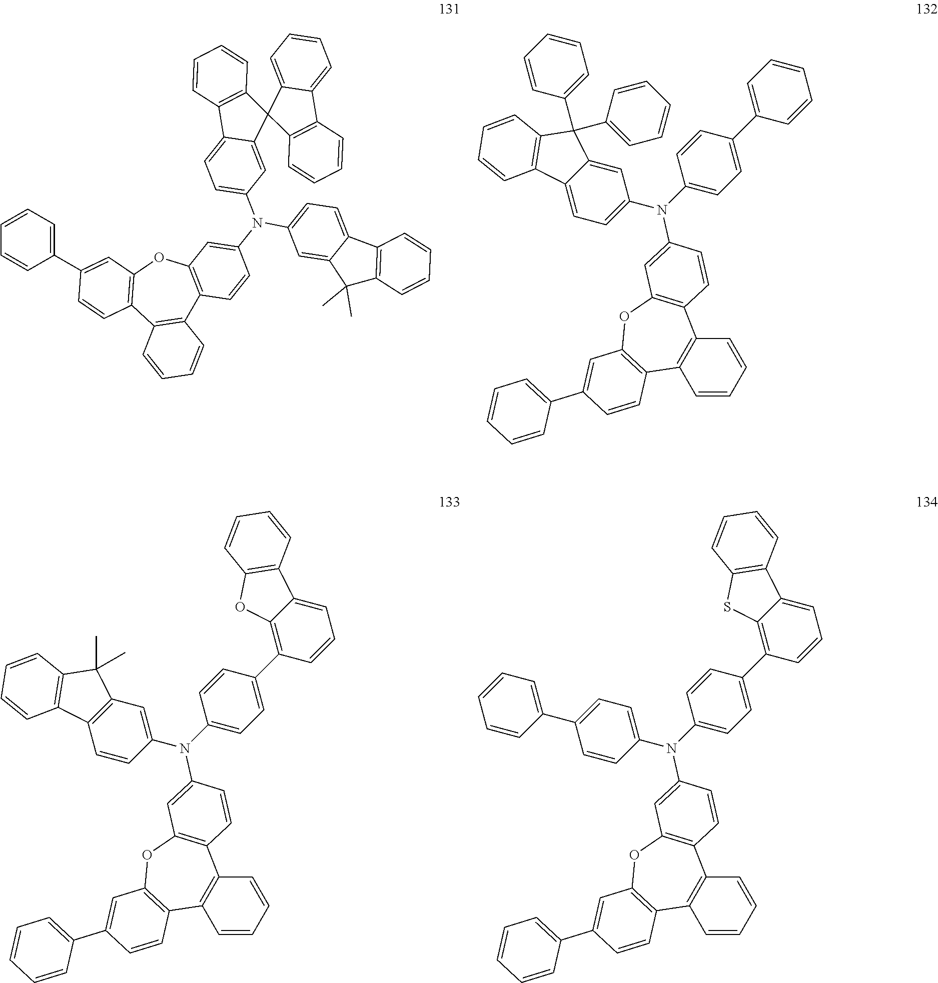 [SMILES: CC1(C)c2ccccc2-c2ccc(N(c3ccc4c(c3)Oc3cc(-c5ccccc5)ccc3-c3ccccc3-4)c3ccc4c(c3)C3(c5ccccc5-c5ccccc53)c3ccccc3-4)cc21 ; c1ccc(-c2ccc(N(c3ccc4c(c3)Oc3cc(-c5ccccc5)ccc3-c3ccccc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc(-c5ccccc5)ccc3-c3ccccc3-4)cc21 ; c1ccc(-c2ccc(N(c3ccc(-c4cccc5c4sc4ccccc45)cc3)c3ccc4c(c3)Oc3cc(-c5ccccc5)ccc3-c3ccccc3-4)cc2)cc1]

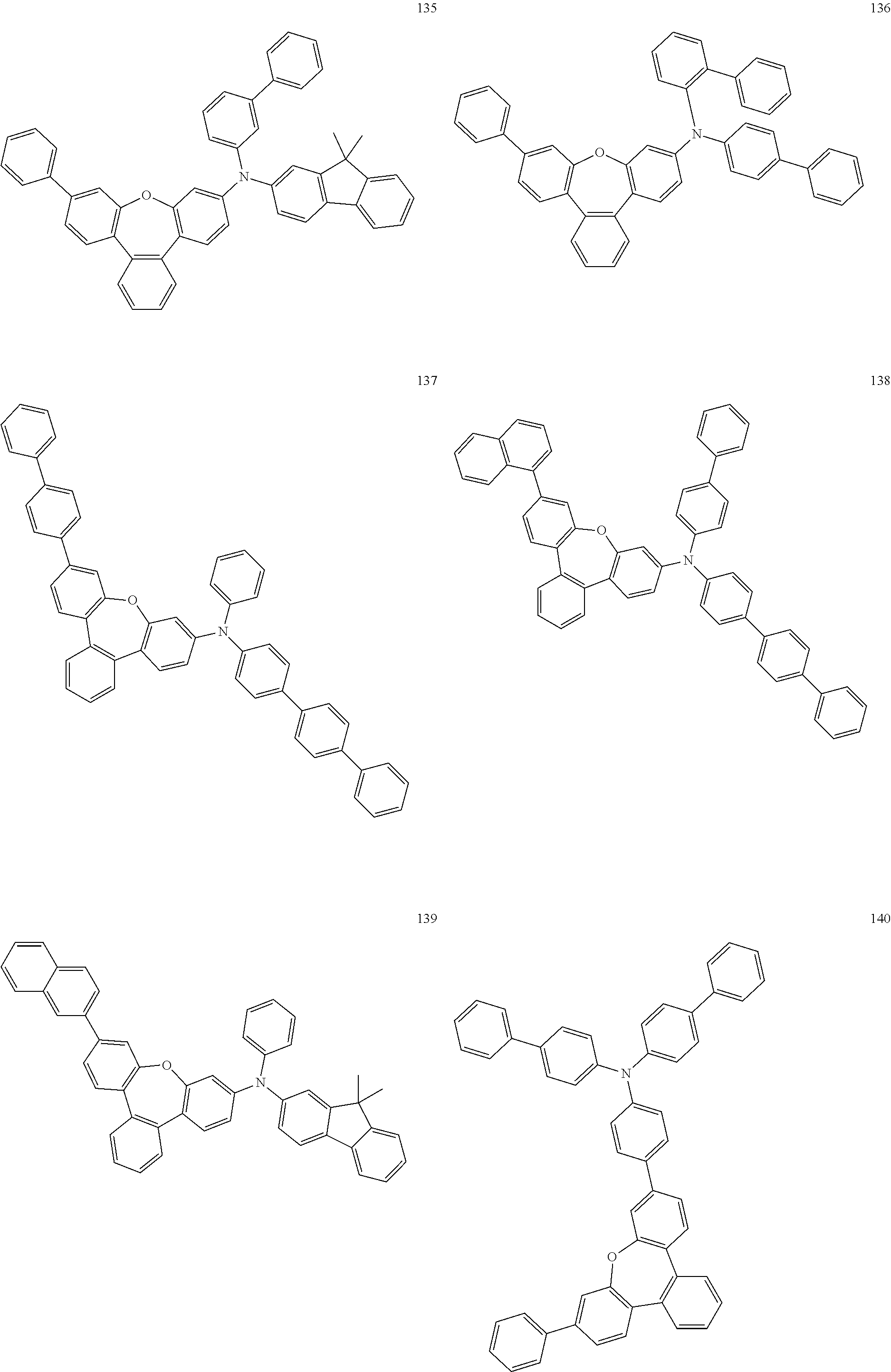

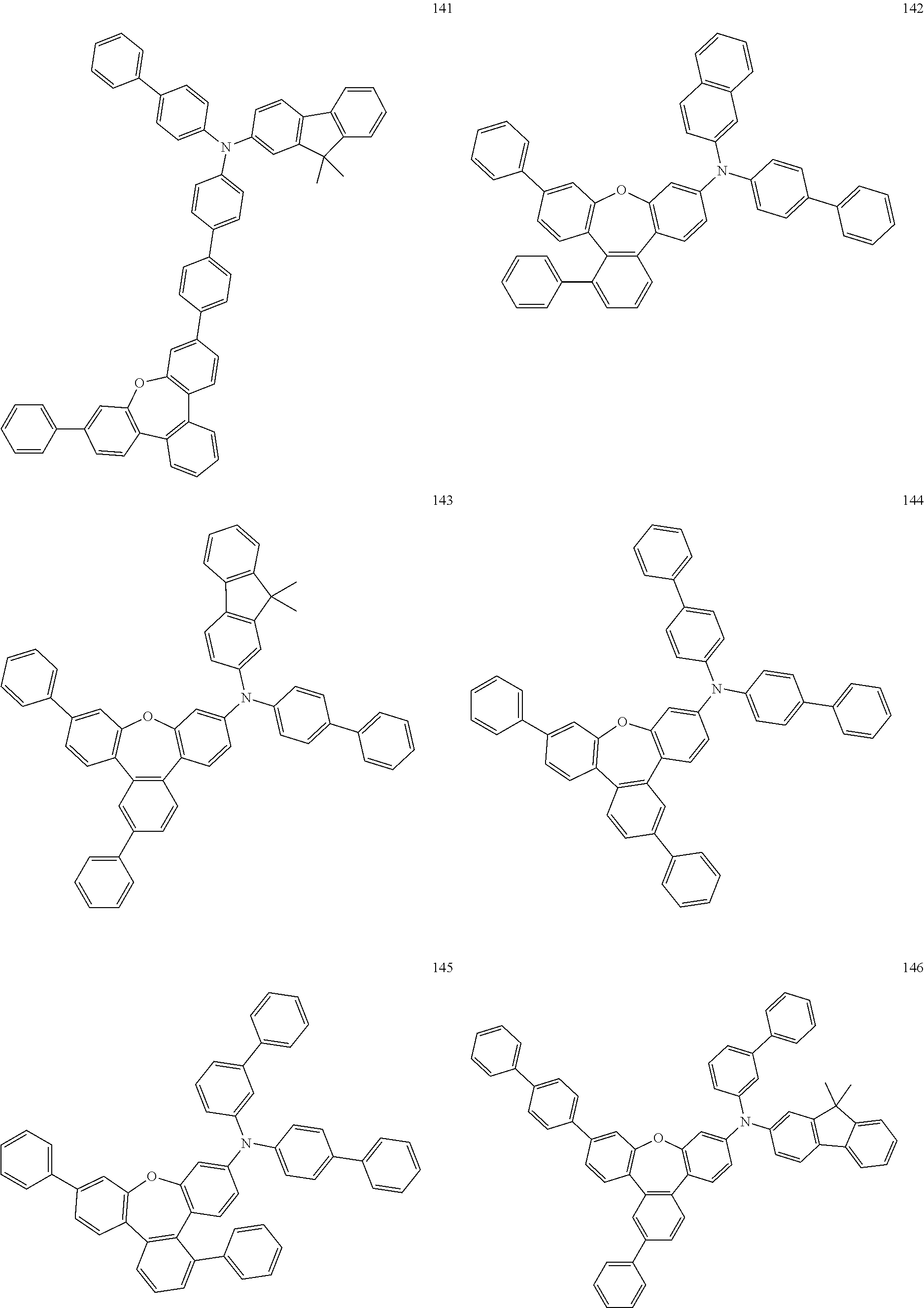

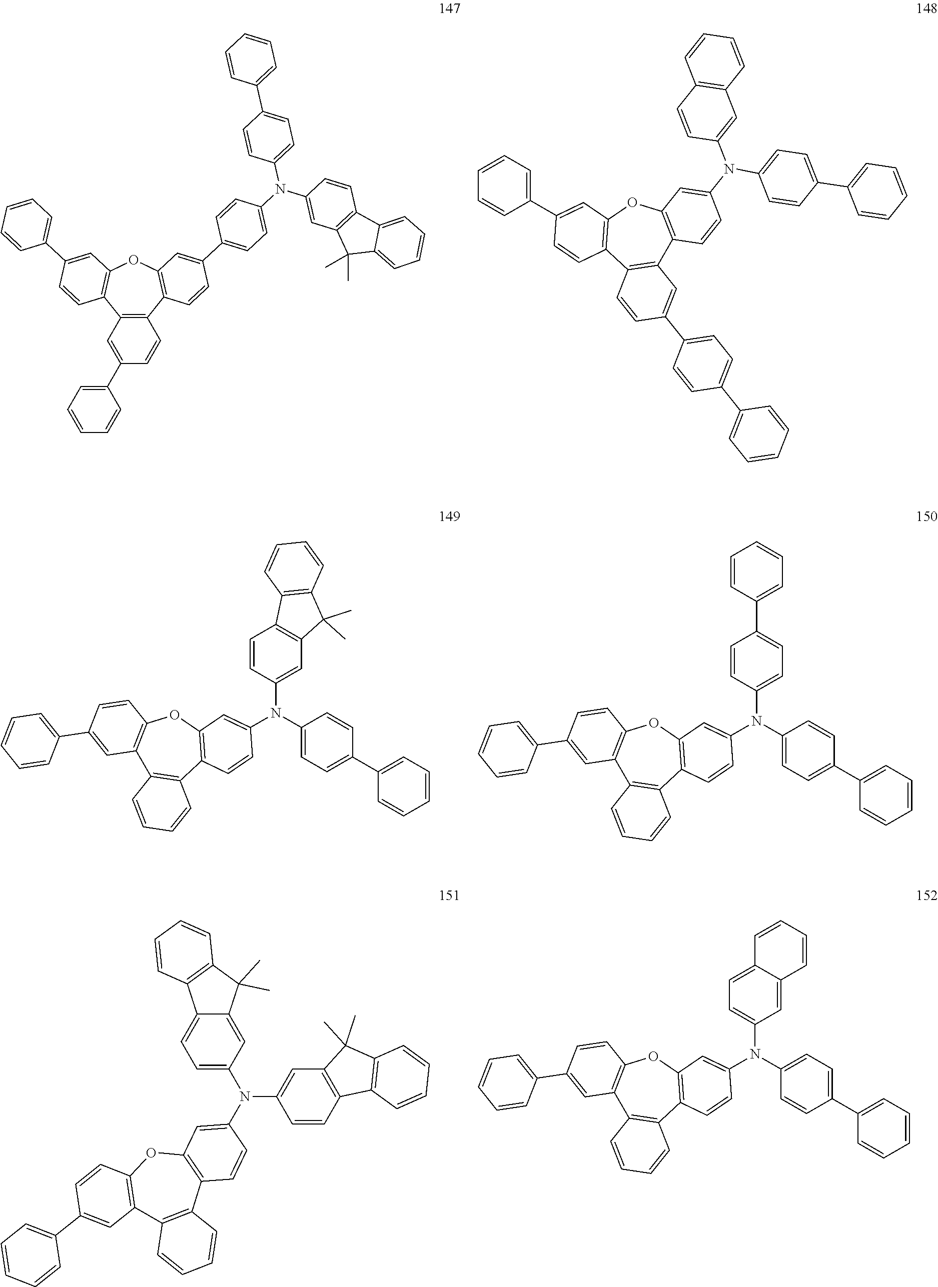

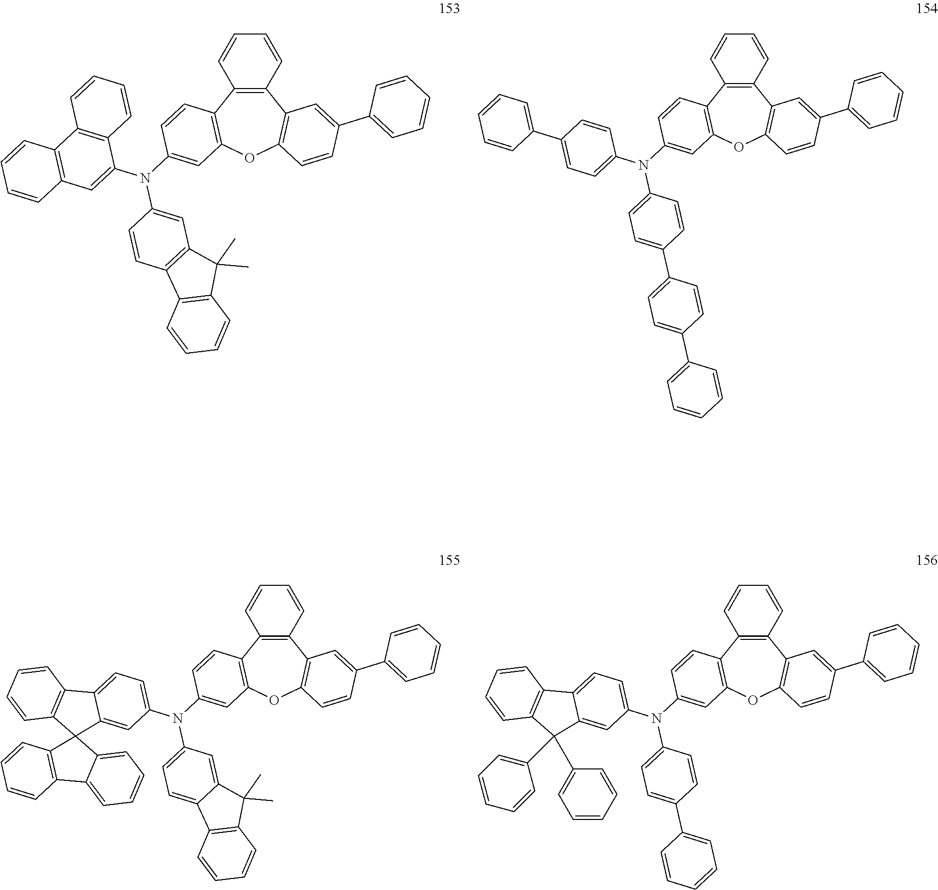 [SMILES: CC1(C)c2ccccc2-c2ccc(N(c3ccc4c(c3)Oc3ccc(-c5ccccc5)cc3-c3ccccc3-4)c3cc4ccccc4c4ccccc34)cc21 ; c1ccc(-c2ccc(-c3ccc(N(c4ccc(-c5ccccc5)cc4)c4ccc5c(c4)Oc4ccc(-c6ccccc6)cc4-c4ccccc4-5)cc3)cc2)cc1 ; CC1(C)c2ccccc2-c2ccc(N(c3ccc4c(c3)Oc3ccc(-c5ccccc5)cc3-c3ccccc3-4)c3ccc4c(c3)C3(c5ccccc5-c5ccccc53)c3ccccc3-4)cc21 ; c1ccc(-c2ccc(N(c3ccc4c(c3)Oc3ccc(-c5ccccc5)cc3-c3ccccc3-4)c3ccc4c(c3)C(c3ccccc3)(c3ccccc3)c3ccccc3-4)cc2)cc1]

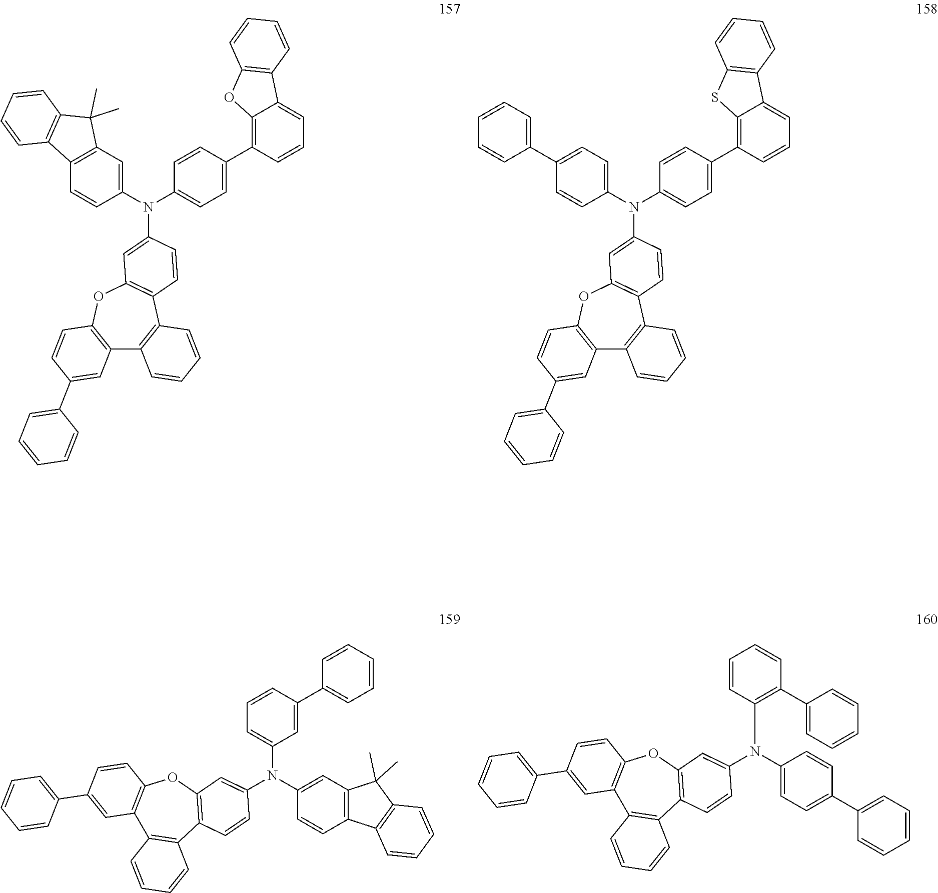 [SMILES: CC1(C)c2ccccc2-c2ccc(N(c3ccc(-c4cccc5c4oc4ccccc45)cc3)c3ccc4c(c3)Oc3ccc(-c5ccccc5)cc3-c3ccccc3-4)cc21 ; c1ccc(-c2ccc(N(c3ccc(-c4cccc5c4sc4ccccc45)cc3)c3ccc4c(c3)Oc3ccc(-c5ccccc5)cc3-c3ccccc3-4)cc2)cc1 ; CC1(C)c2ccccc2-c2ccc(N(c3cccc(-c4ccccc4)c3)c3ccc4c(c3)Oc3ccc(-c5ccccc5)cc3-c3ccccc3-4)cc21 ; c1ccc(-c2ccc(N(c3ccc4c(c3)Oc3ccc(-c5ccccc5)cc3-c3ccccc3-4)c3ccccc3-c3ccccc3)cc2)cc1]

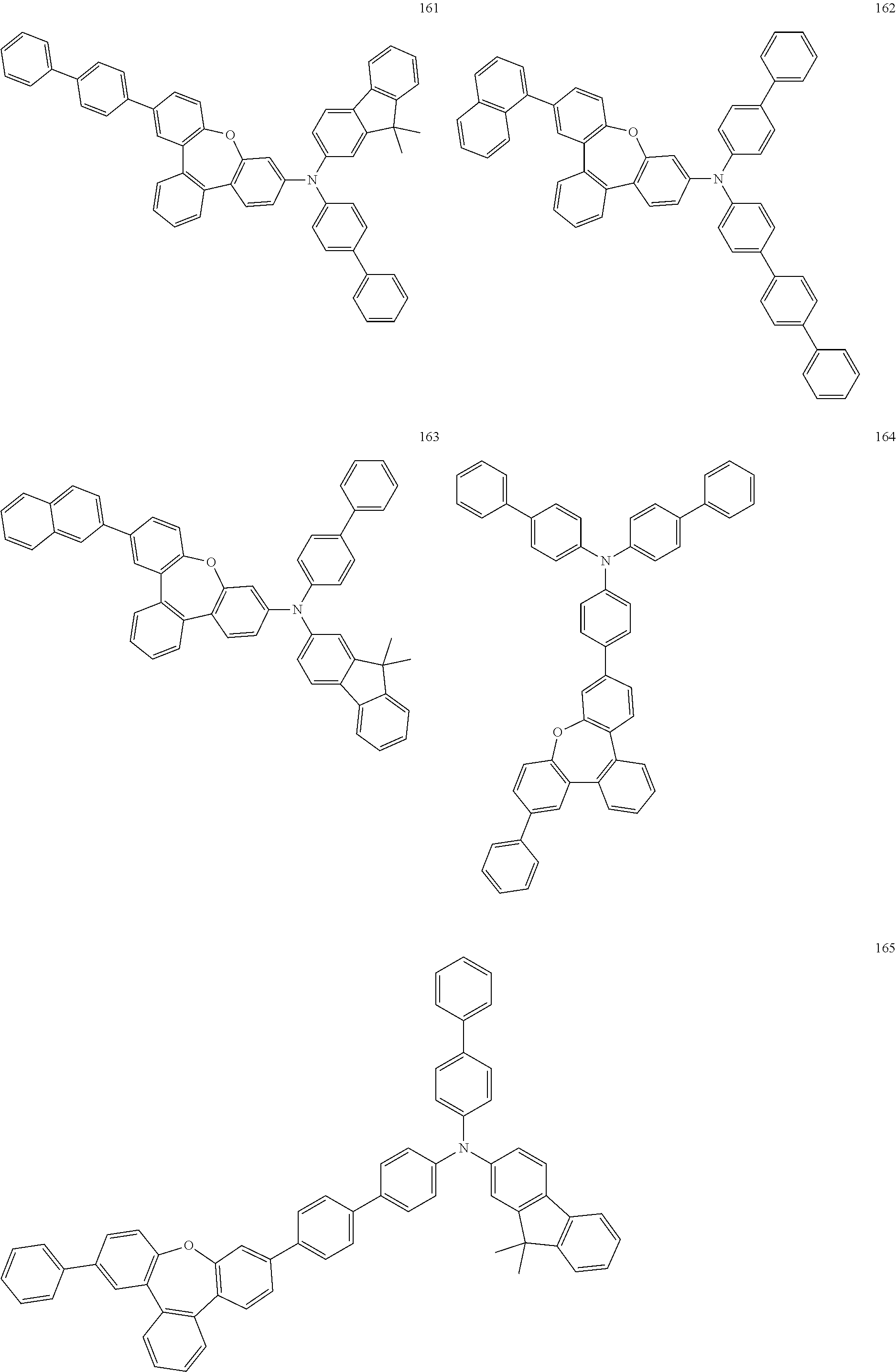

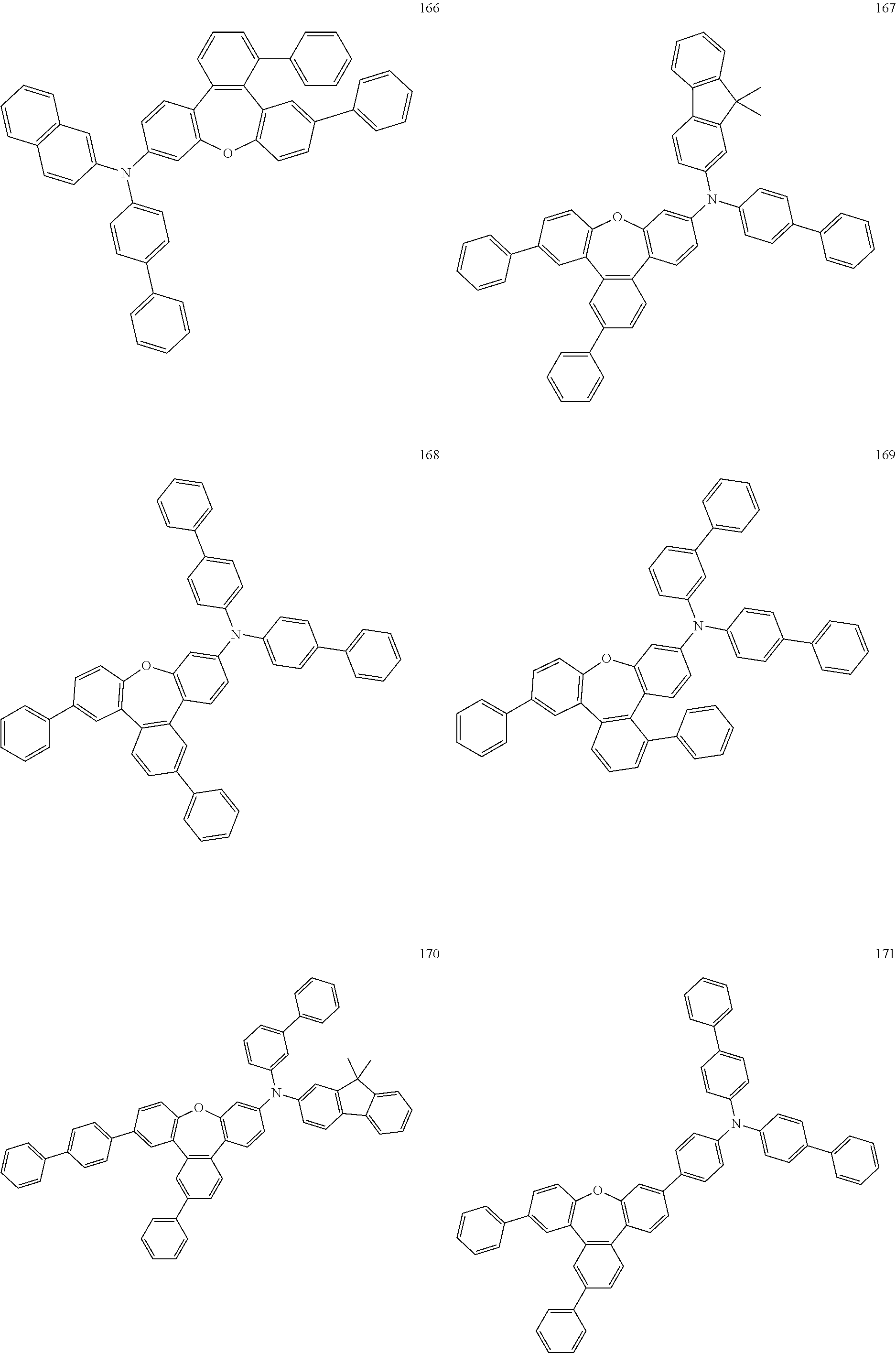

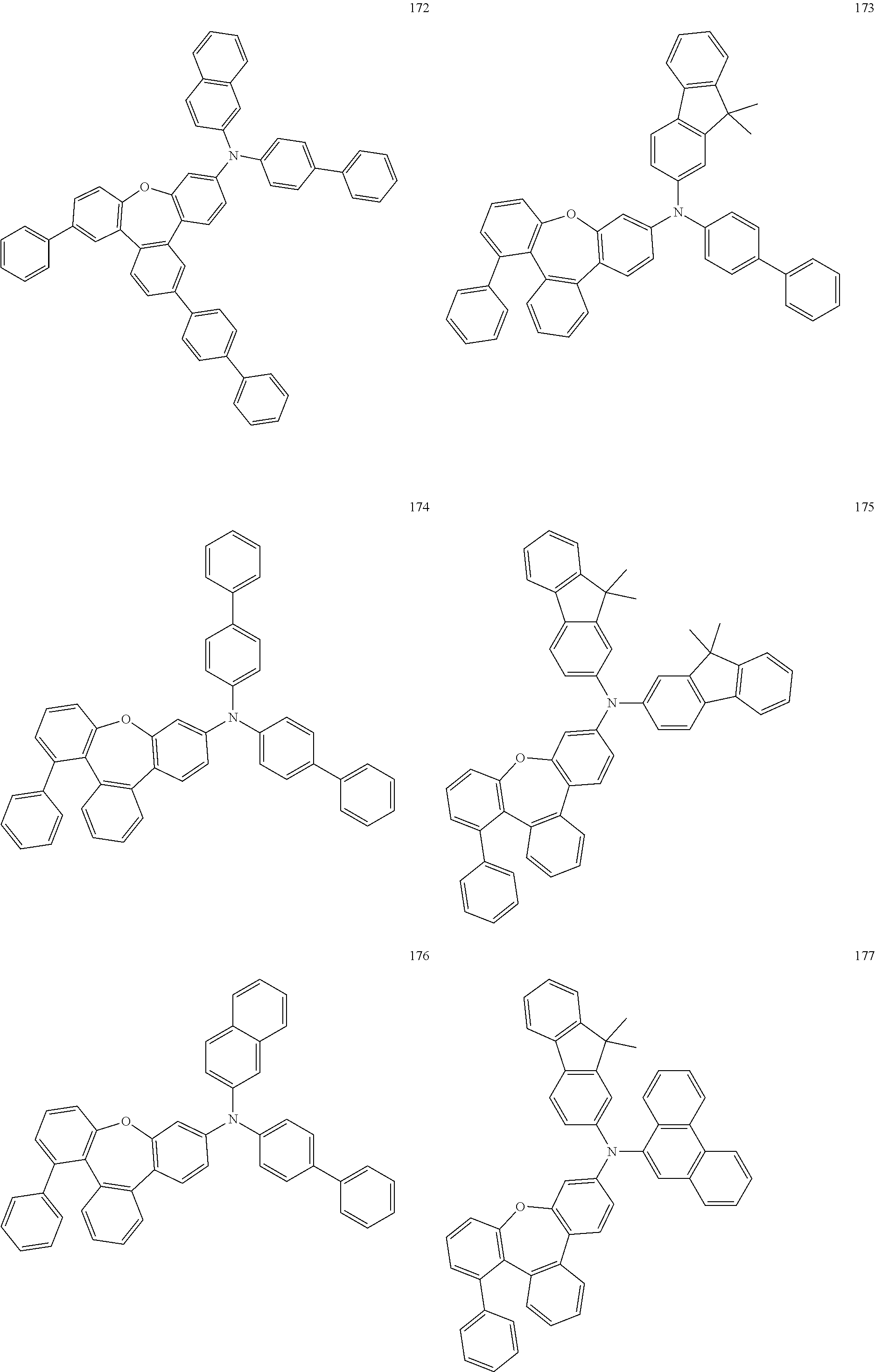

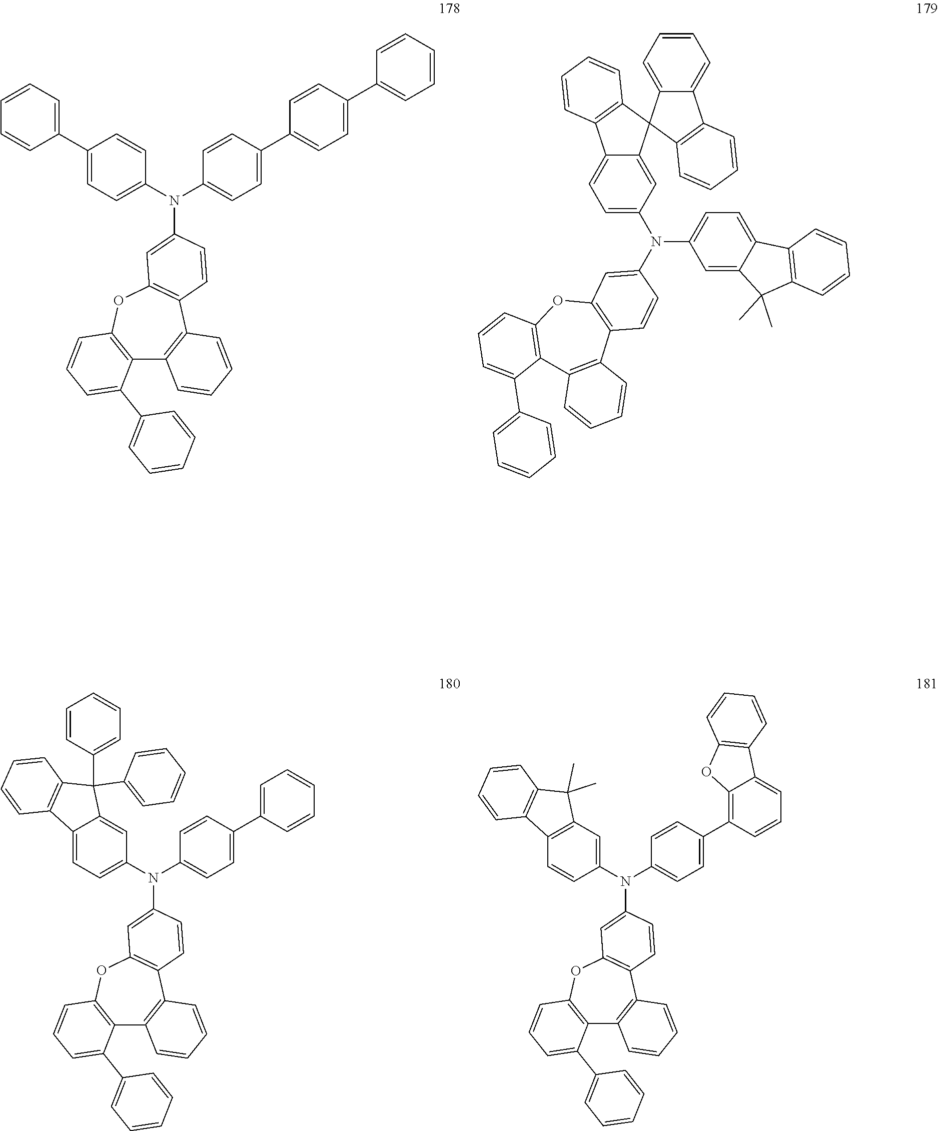 [SMILES: c1ccc(-c2ccc(-c3ccc(N(c4ccc(-c5ccccc5)cc4)c4ccc5c(c4)Oc4cccc(-c6ccccc6)c4-c4ccccc4-5)cc3)cc2)cc1 ; CC1(C)c2ccccc2-c2ccc(N(c3ccc4c(c3)Oc3cccc(-c5ccccc5)c3-c3ccccc3-4)c3ccc4c(c3)C3(c5ccccc5-c5ccccc53)c3ccccc3-4)cc21 ; c1ccc(-c2ccc(N(c3ccc4c(c3)Oc3cccc(-c5ccccc5)c3-c3ccccc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cccc(-c5ccccc5)c3-c3ccccc3-4)cc21]

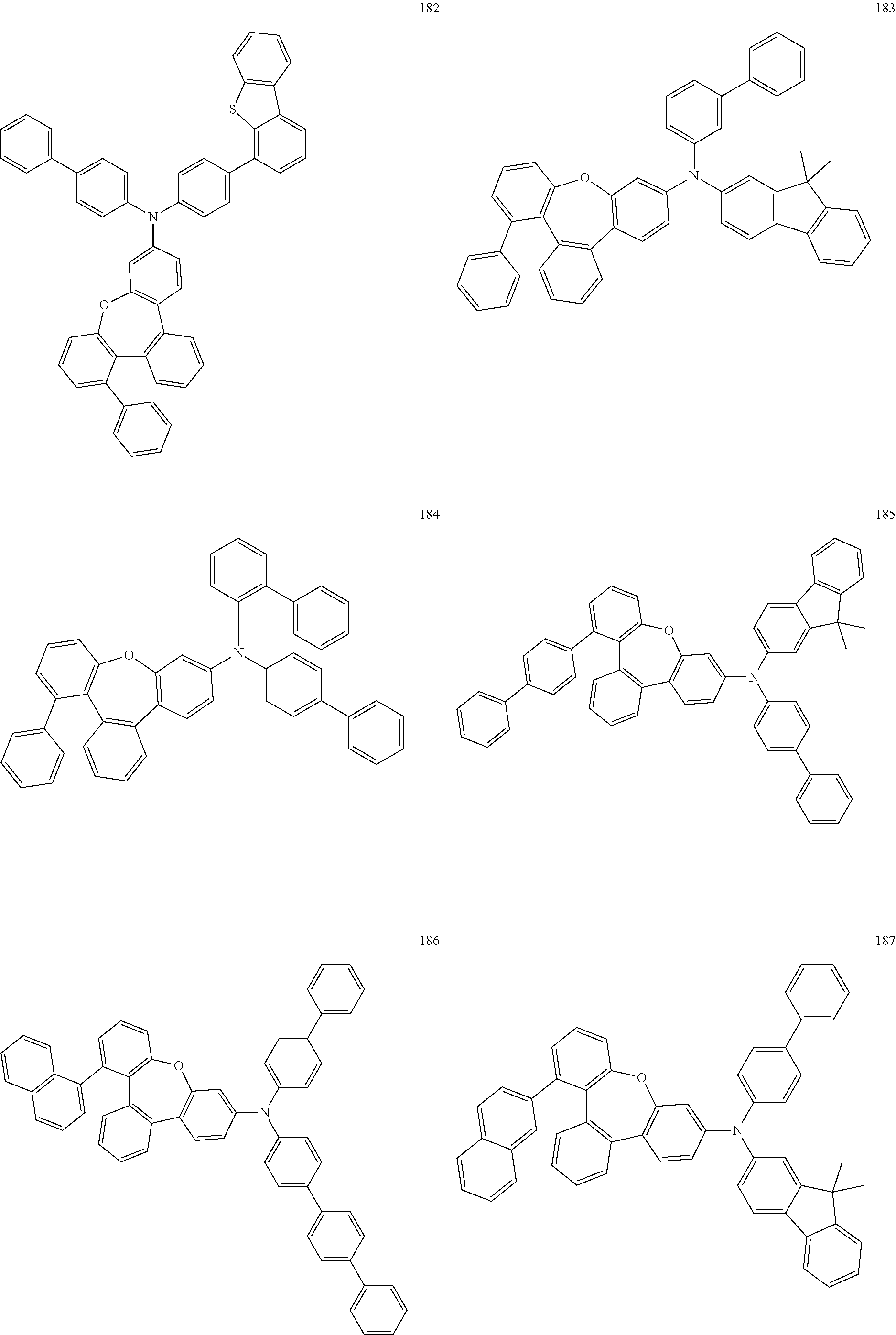

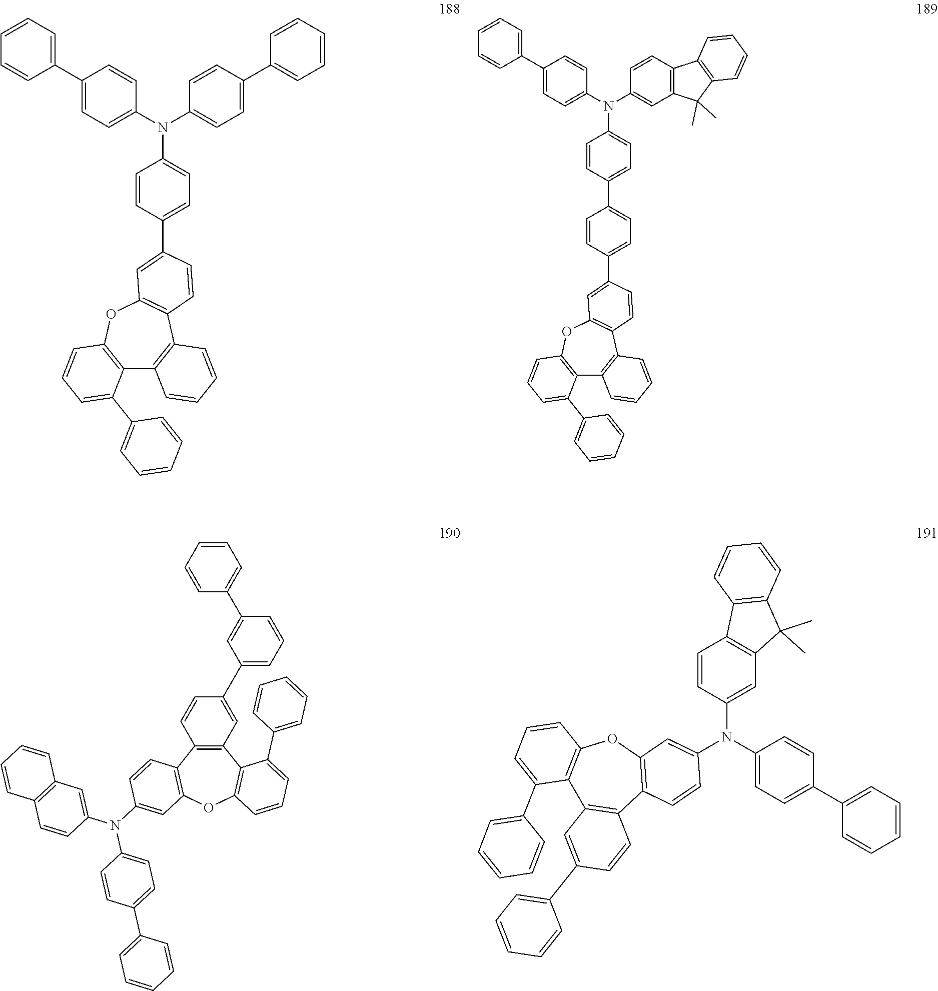 [SMILES: c1ccc(-c2ccc(N(c3ccc(-c4ccccc4)cc3)c3ccc(-c4ccc5c(c4)Oc4cccc(-c6ccccc6)c4-c4ccccc4-5)cc3)cc2)cc1 ; CC1(C)c2ccccc2-c2ccc(N(c3ccc(-c4ccccc4)cc3)c3ccc(-c4ccc(-c5ccc6c(c5)Oc5cccc(-c7ccccc7)c5-c5ccccc5-6)cc4)cc3)cc21 ; c1ccc(-c2ccc(N(c3ccc4c(c3)Oc3cccc(-c5ccccc5)c3-c3cc(-c5cccc(-c6ccccc6)c5)ccc3-4)c3ccc4ccccc4c3)cc2)cc1 ; CC1(C)c2ccccc2-c2ccc(N(c3ccc(-c4ccccc4)cc3)c3ccc4c(c3)Oc3cccc(-c5ccccc5)c3-c3cc(-c5ccccc5)ccc3-4)cc21]

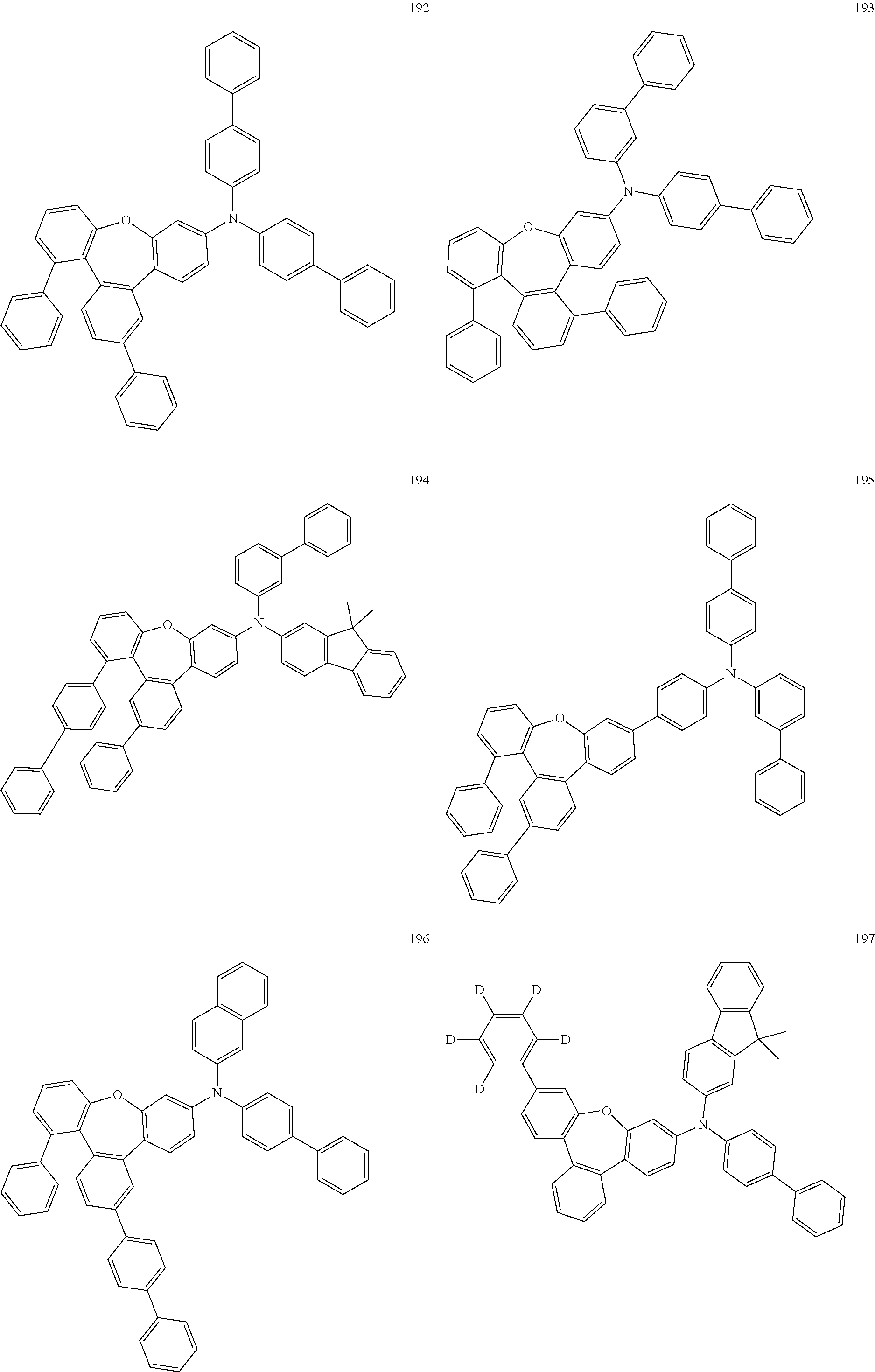

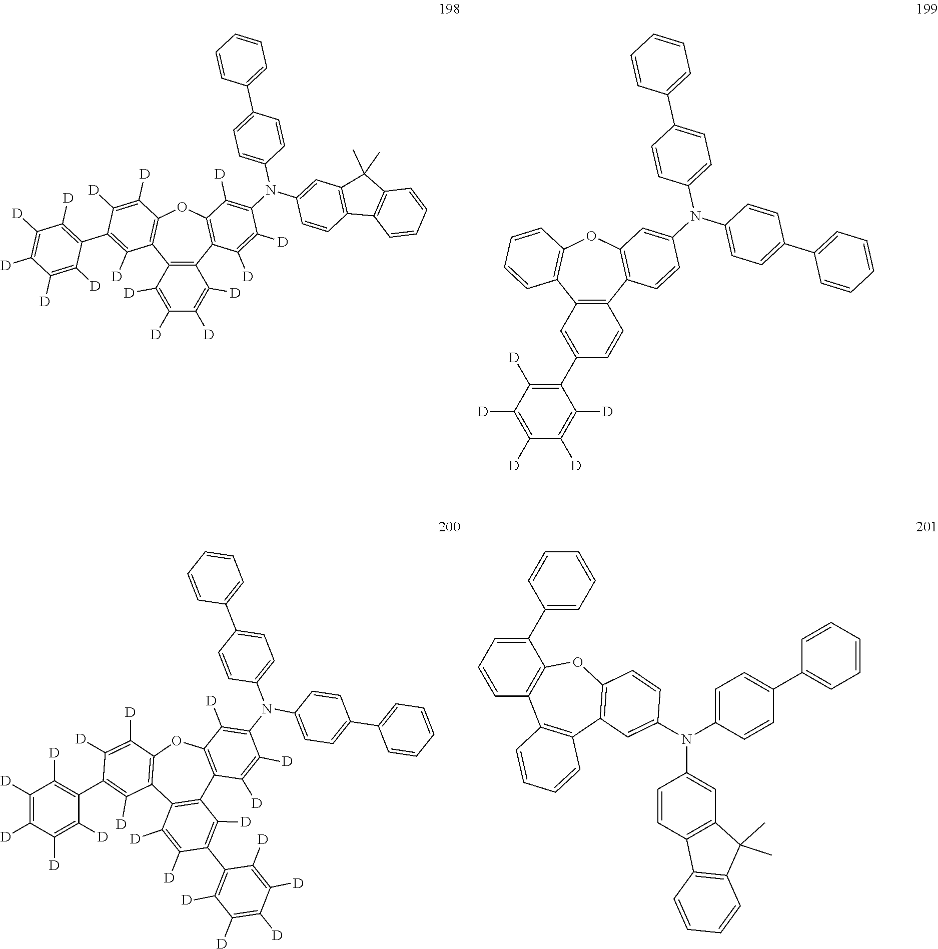

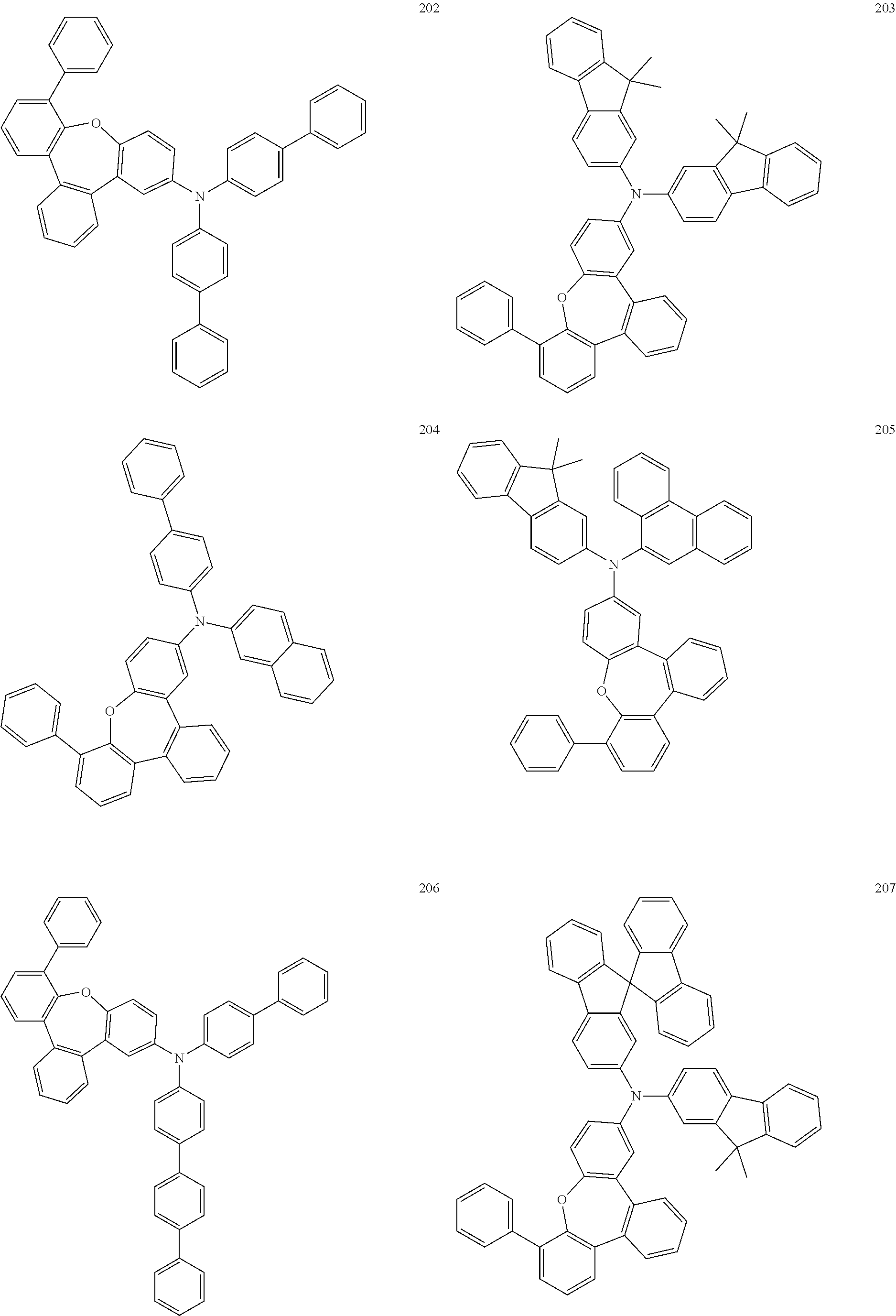

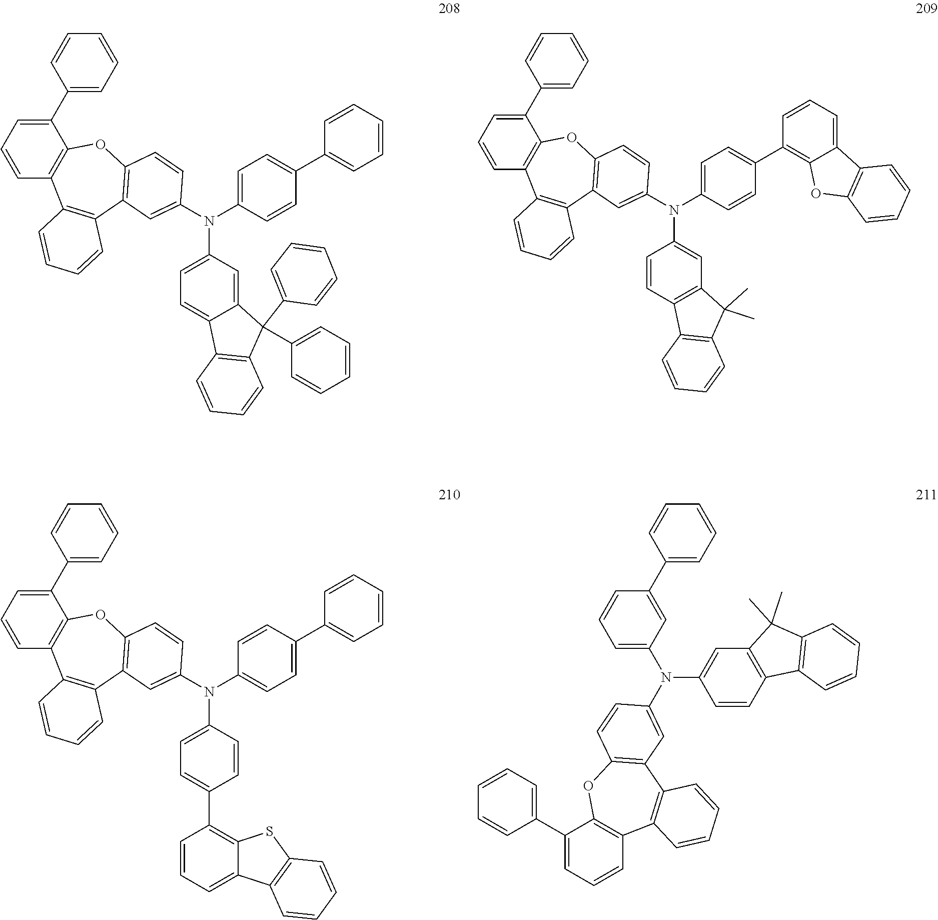 [SMILES: c1ccc(-c2ccc(N(c3ccc4c(c3)-c3ccccc3-c3cccc(-c5ccccc5)c3O4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)-c3ccccc3-c3cccc(-c5ccccc5)c3O4)cc21 ; c1ccc(-c2ccc(N(c3ccc(-c4cccc5c4sc4ccccc45)cc3)c3ccc4c(c3)-c3ccccc3-c3cccc(-c5ccccc5)c3O4)cc2)cc1 ; CC1(C)c2ccccc2-c2ccc(N(c3cccc(-c4ccccc4)c3)c3ccc4c(c3)-c3ccccc3-c3cccc(-c5ccccc5)c3O4)cc21]

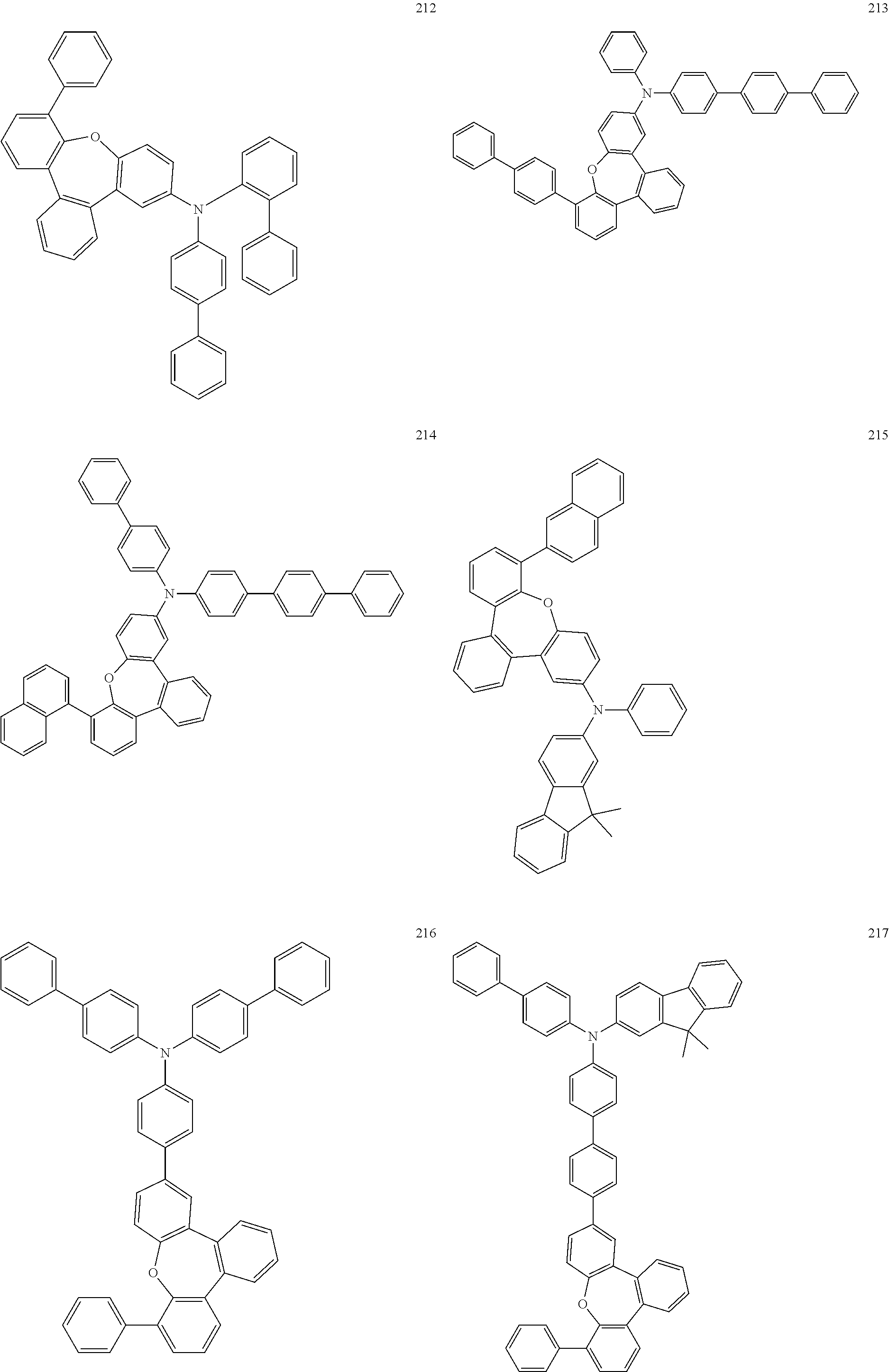

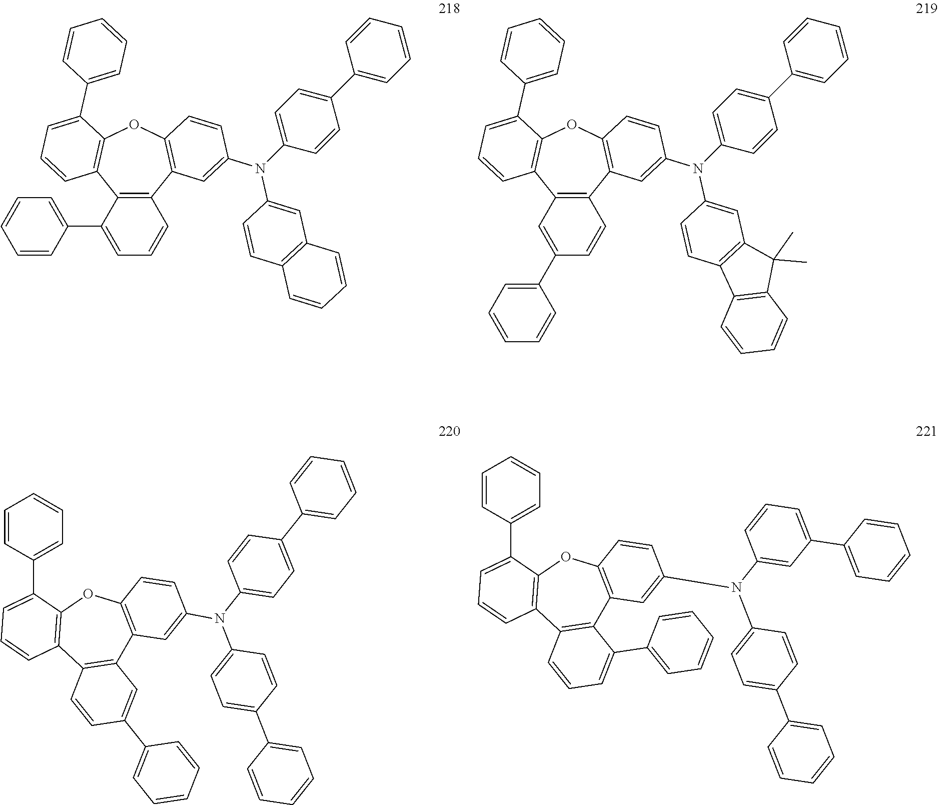 [SMILES: c1ccc(-c2ccc(N(c3ccc4c(c3)-c3cccc(-c5ccccc5)c3-c3cccc(-c5ccccc5)c3O4)c3ccc4ccccc4c3)cc2)cc1 ; CC1(C)c2ccccc2-c2ccc(N(c3ccc(-c4ccccc4)cc3)c3ccc4c(c3)-c3ccc(-c5ccccc5)cc3-c3cccc(-c5ccccc5)c3O4)cc21 ; c1ccc(-c2ccc(N(c3ccc(-c4ccccc4)cc3)c3ccc4c(c3)-c3cc(-c5ccccc5)ccc3-c3cccc(-c5ccccc5)c3O4)cc2)cc1 ; c1ccc(-c2ccc(N(c3cccc(-c4ccccc4)c3)c3ccc4c(c3)-c3c(-c5ccccc5)cccc3-c3cccc(-c5ccccc5)c3O4)cc2)cc1]

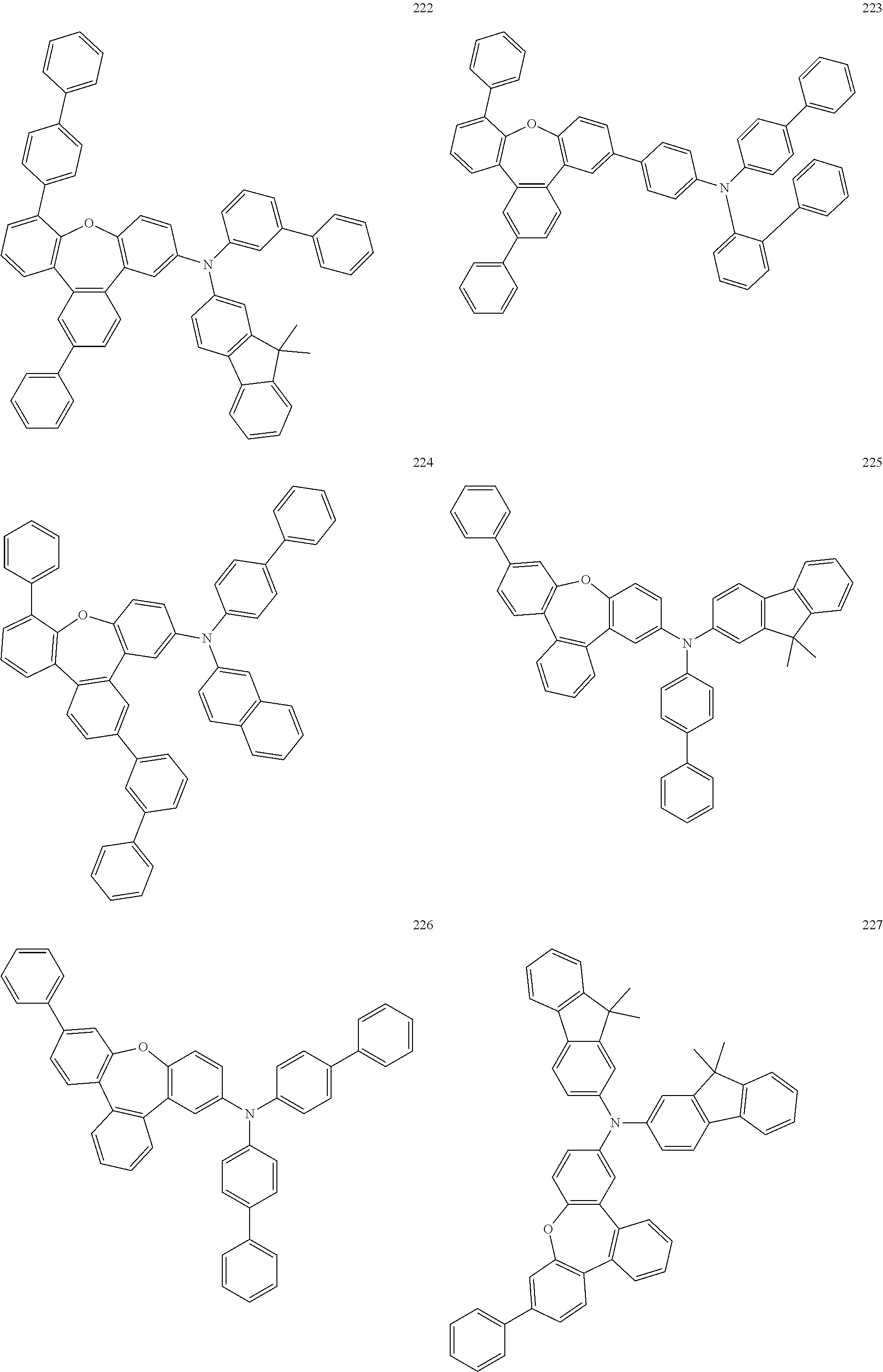

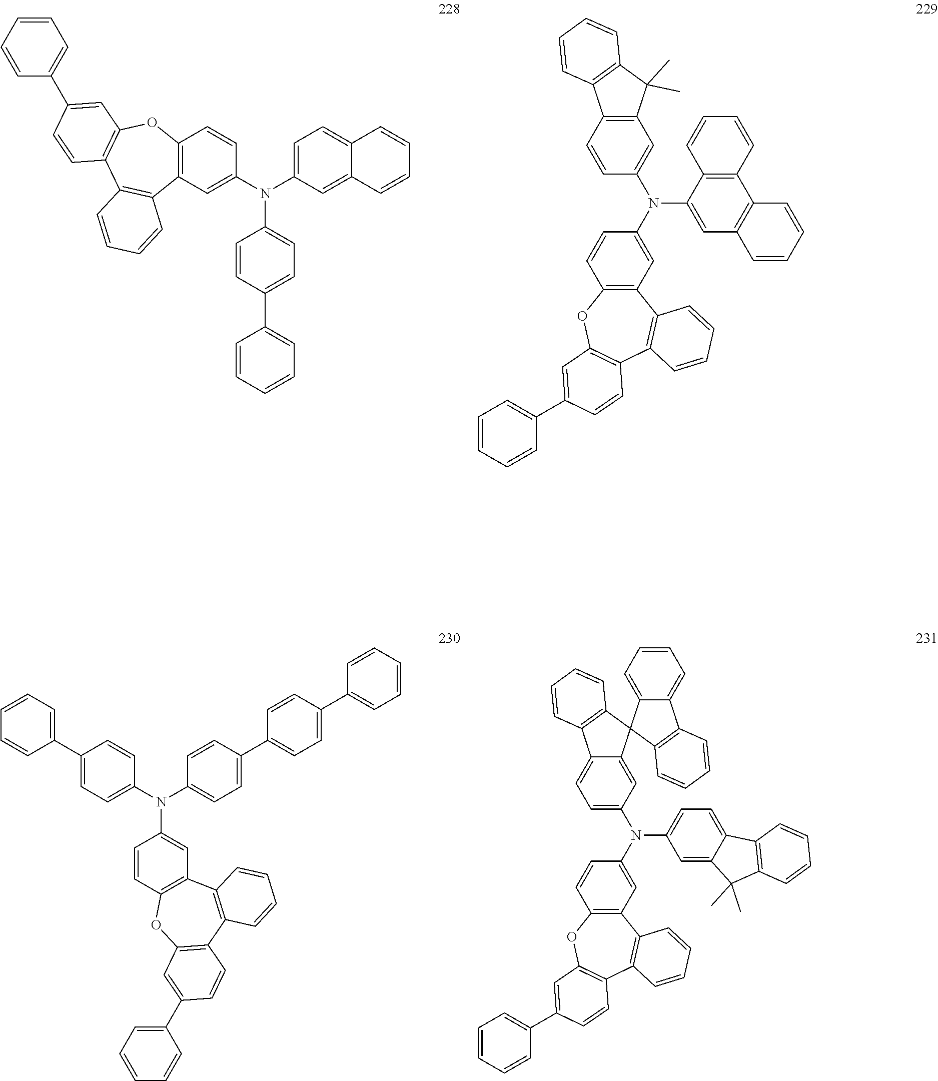 [SMILES: c1ccc(-c2ccc(N(c3ccc4c(c3)-c3ccccc3-c3ccc(-c5ccccc5)cc3O4)c3ccc4ccccc4c3)cc2)cc1 ; CC1(C)c2ccccc2-c2ccc(N(c3ccc4c(c3)-c3ccccc3-c3ccc(-c5ccccc5)cc3O4)c3cc4ccccc4c4ccccc34)cc21 ; c1ccc(-c2ccc(-c3ccc(N(c4ccc(-c5ccccc5)cc4)c4ccc5c(c4)-c4ccccc4-c4ccc(-c6ccccc6)cc4O5)cc3)cc2)cc1 ; CC1(C)c2ccccc2-c2ccc(N(c3ccc4c(c3)-c3ccccc3-c3ccc(-c5ccccc5)cc3O4)c3ccc4c(c3)C3(c5ccccc5-c5ccccc53)c3ccccc3-4)cc21]

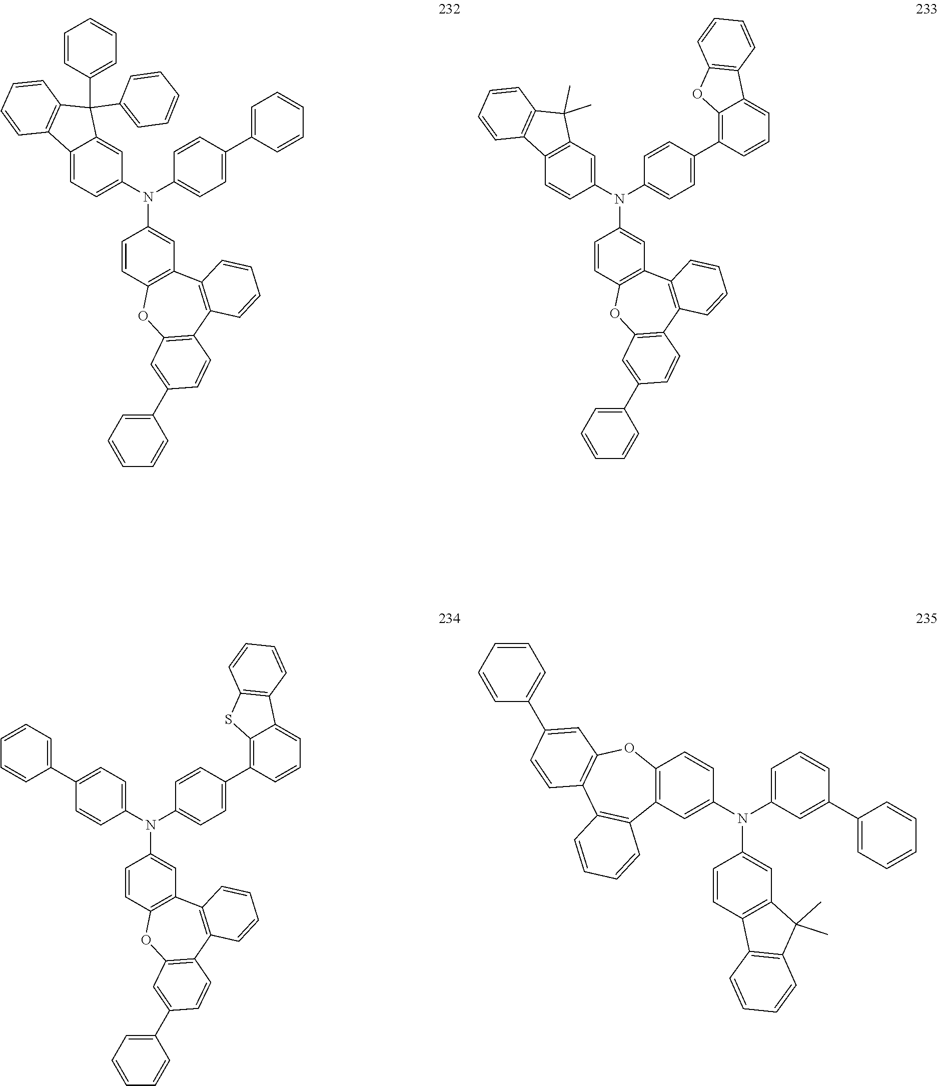 [SMILES: c1ccc(-c2ccc(N(c3ccc4c(c3)-c3ccccc3-c3ccc(-c5ccccc5)cc3O4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)-c3ccccc3-c3ccc(-c5ccccc5)cc3O4)cc21 ; c1ccc(-c2ccc(N(c3ccc(-c4cccc5c4sc4ccccc45)cc3)c3ccc4c(c3)-c3ccccc3-c3ccc(-c5ccccc5)cc3O4)cc2)cc1 ; CC1(C)c2ccccc2-c2ccc(N(c3cccc(-c4ccccc4)c3)c3ccc4c(c3)-c3ccccc3-c3ccc(-c5ccccc5)cc3O4)cc21]

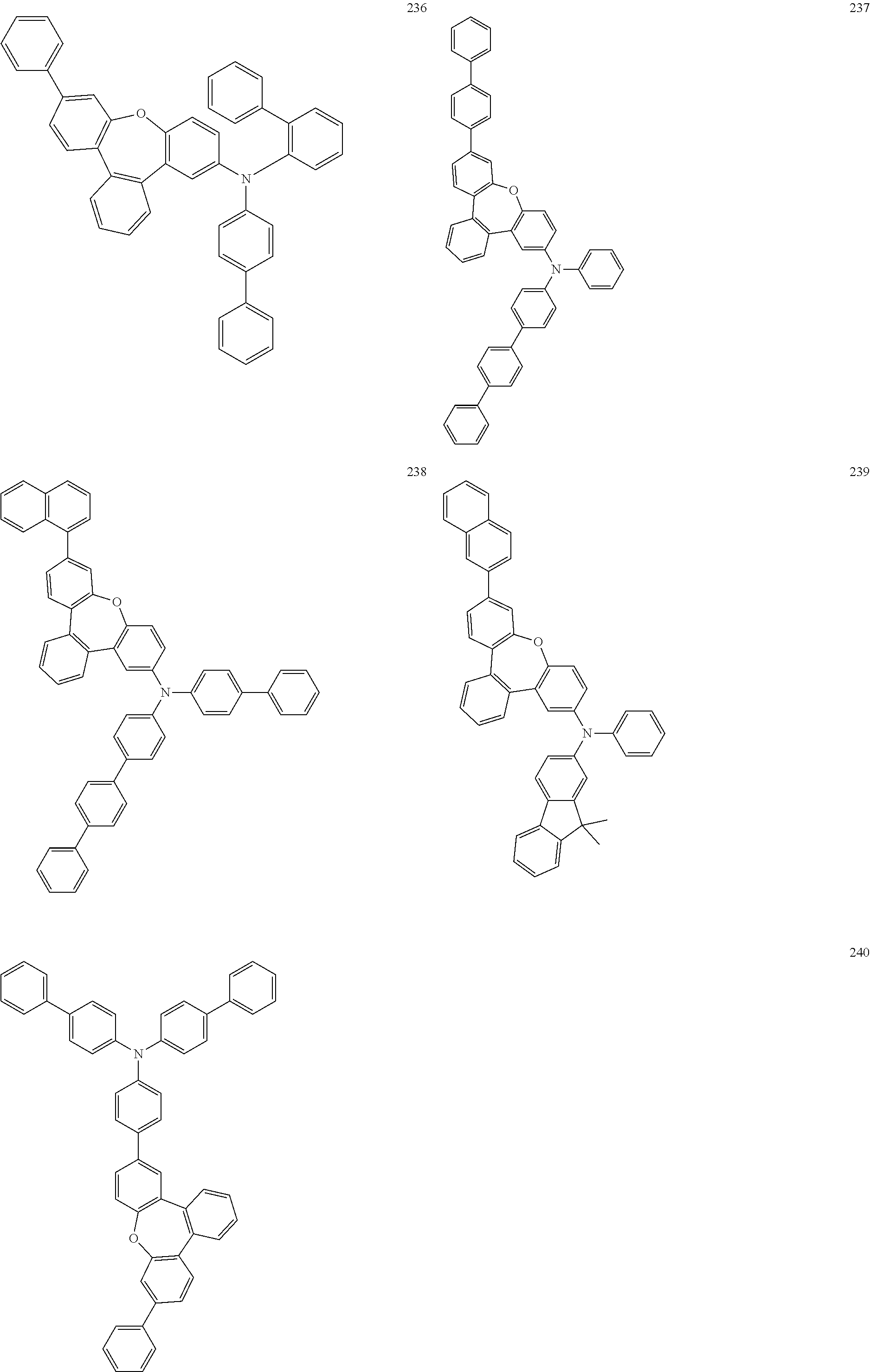 [SMILES: c1ccc(-c2ccc(N(c3ccc4c(c3)-c3ccccc3-c3ccc(-c5ccccc5)cc3O4)c3ccccc3-c3ccccc3)cc2)cc1 ; c1ccc(-c2ccc(-c3ccc(N(c4ccccc4)c4ccc5c(c4)-c4ccccc4-c4ccc(-c6ccc(-c7ccccc7)cc6)cc4O5)cc3)cc2)cc1 ; c1ccc(-c2ccc(-c3ccc(N(c4ccc(-c5ccccc5)cc4)c4ccc5c(c4)-c4ccccc4-c4ccc(-c6cccc7ccccc67)cc4O5)cc3)cc2)cc1 ; CC1(C)c2ccccc2-c2ccc(N(c3ccccc3)c3ccc4c(c3)-c3ccccc3-c3ccc(-c5ccc6ccccc6c5)cc3O4)cc21 ; c1ccc(-c2ccc(N(c3ccc(-c4ccccc4)cc3)c3ccc(-c4ccc5c(c4)-c4ccccc4-c4ccc(-c6ccccc6)cc4O5)cc3)cc2)cc1]

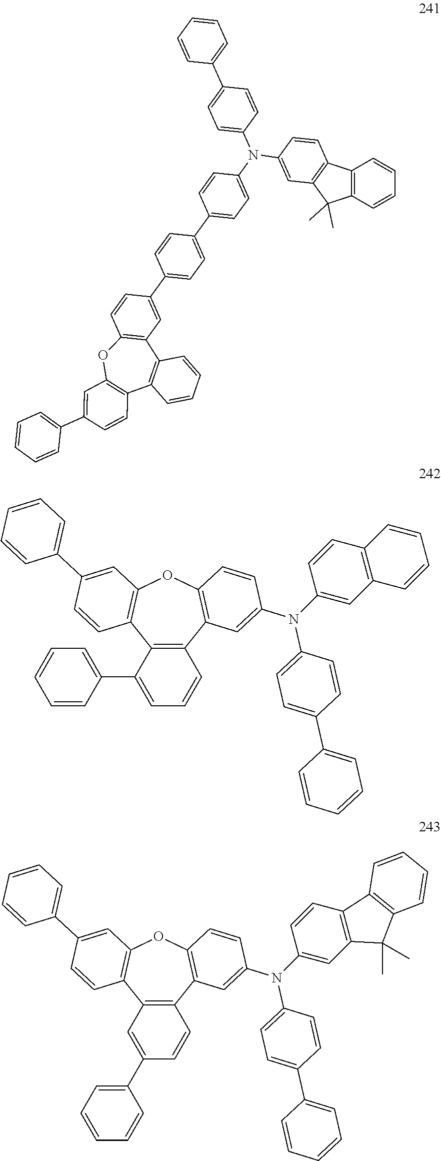

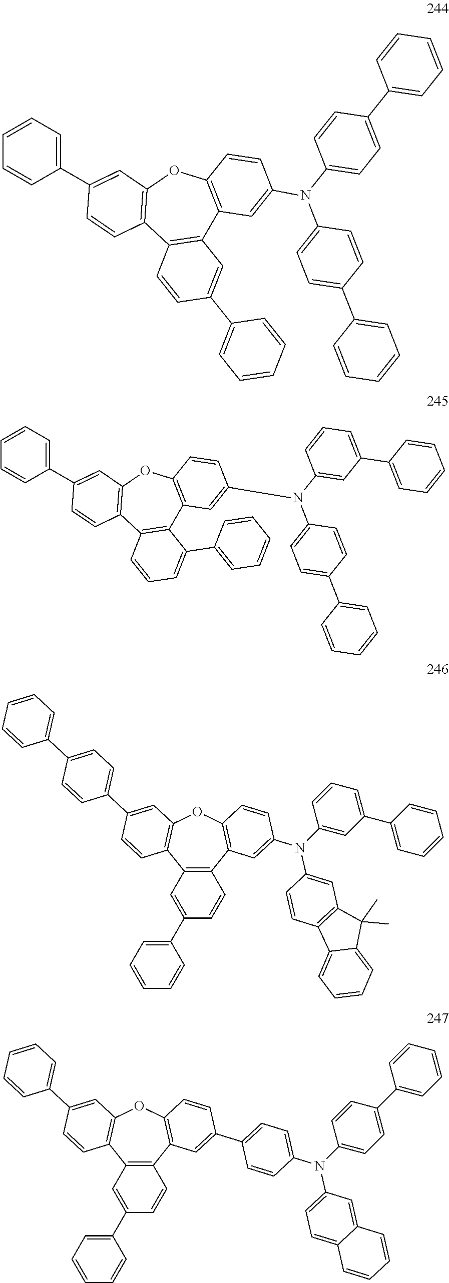 [SMILES: c1ccc(-c2ccc(N(c3ccc(-c4ccccc4)cc3)c3ccc4c(c3)-c3cc(-c5ccccc5)ccc3-c3ccc(-c5ccccc5)cc3O4)cc2)cc1 ; c1ccc(-c2ccc(N(c3cccc(-c4ccccc4)c3)c3ccc4c(c3)-c3c(-c5ccccc5)cccc3-c3ccc(-c5ccccc5)cc3O4)cc2)cc1 ; CC1(C)c2ccccc2-c2ccc(N(c3cccc(-c4ccccc4)c3)c3ccc4c(c3)-c3ccc(-c5ccccc5)cc3-c3ccc(-c5ccc(-c6ccccc6)cc5)cc3O4)cc21 ; c1ccc(-c2ccc(N(c3ccc(-c4ccc5c(c4)-c4ccc(-c6ccccc6)cc4-c4ccc(-c6ccccc6)cc4O5)cc3)c3ccc4ccccc4c3)cc2)cc1]

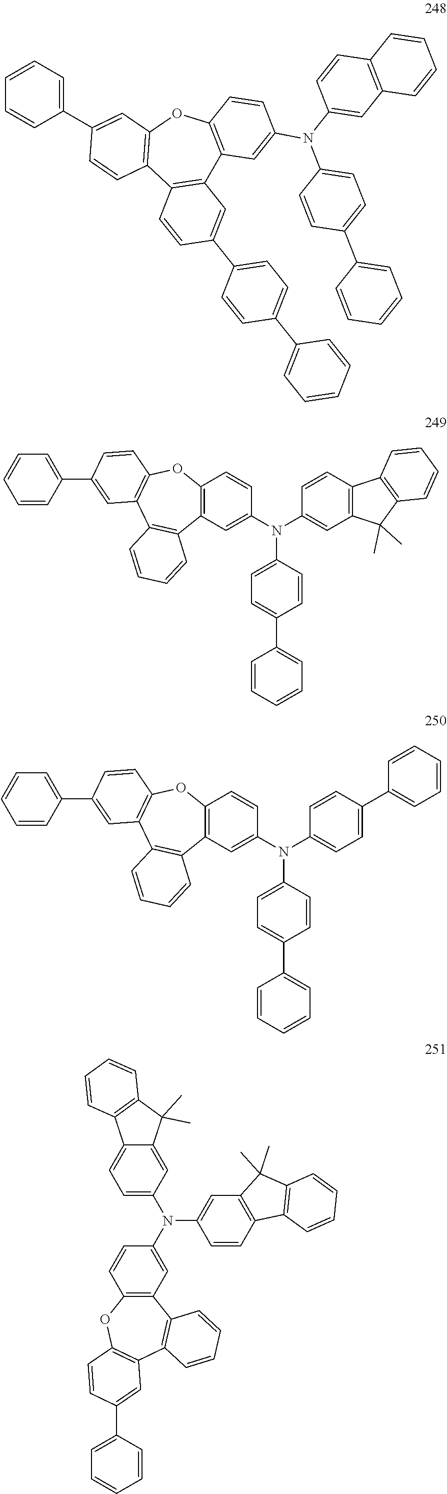 [SMILES: c1ccc(-c2ccc(-c3ccc4c(c3)-c3cc(N(c5ccc(-c6ccccc6)cc5)c5ccc6ccccc6c5)ccc3Oc3cc(-c5ccccc5)ccc3-4)cc2)cc1 ; CC1(C)c2ccccc2-c2ccc(N(c3ccc(-c4ccccc4)cc3)c3ccc4c(c3)-c3ccccc3-c3cc(-c5ccccc5)ccc3O4)cc21 ; c1ccc(-c2ccc(N(c3ccc(-c4ccccc4)cc3)c3ccc4c(c3)-c3ccccc3-c3cc(-c5ccccc5)ccc3O4)cc2)cc1 ; CC1(C)c2ccccc2-c2ccc(N(c3ccc4c(c3)-c3ccccc3-c3cc(-c5ccccc5)ccc3O4)c3ccc4c(c3)C(C)(C)c3ccccc3-4)cc21]

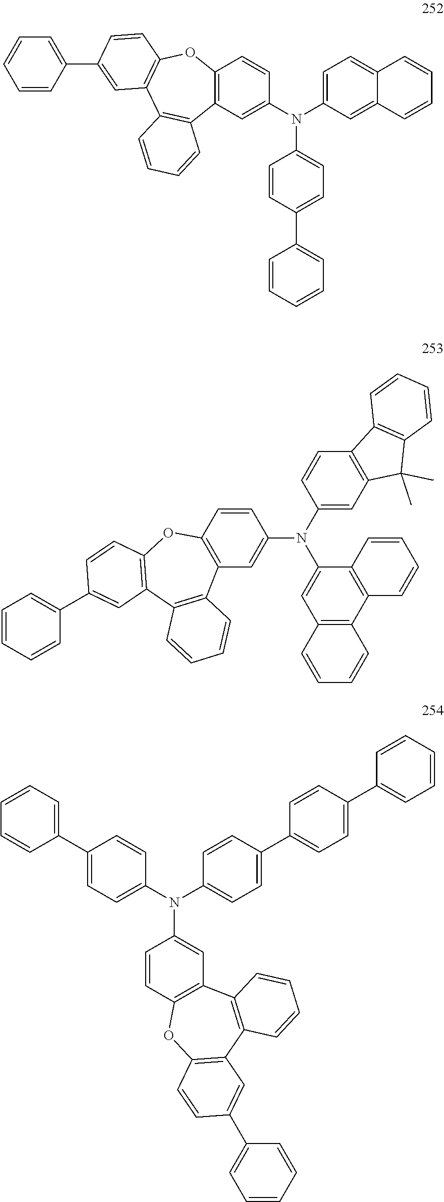 [SMILES: c1ccc(-c2ccc(N(c3ccc4c(c3)-c3ccccc3-c3cc(-c5ccccc5)ccc3O4)c3ccc4ccccc4c3)cc2)cc1 ; CC1(C)c2ccccc2-c2ccc(N(c3ccc4c(c3)-c3ccccc3-c3cc(-c5ccccc5)ccc3O4)c3cc4ccccc4c4ccccc34)cc21 ; c1ccc(-c2ccc(-c3ccc(N(c4ccc(-c5ccccc5)cc4)c4ccc5c(c4)-c4ccccc4-c4cc(-c6ccccc6)ccc4O5)cc3)cc2)cc1]

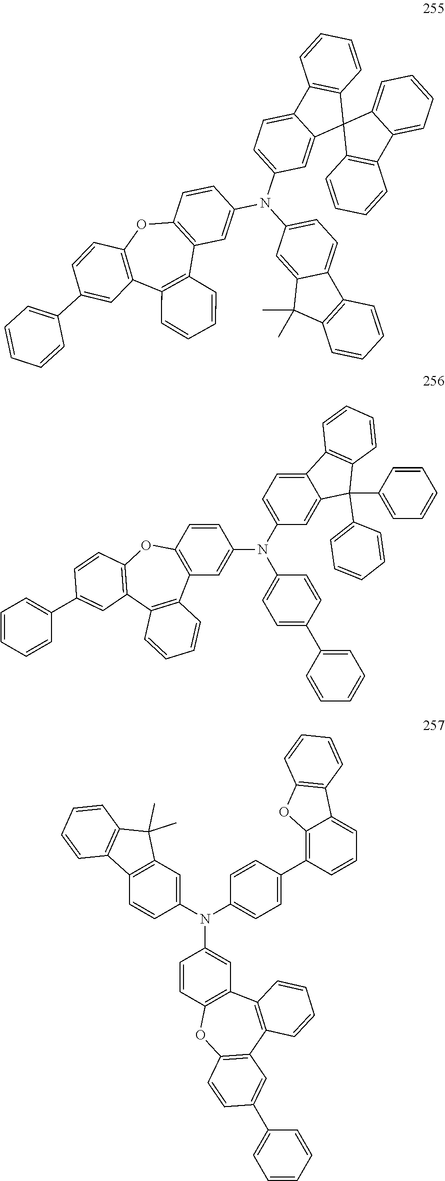

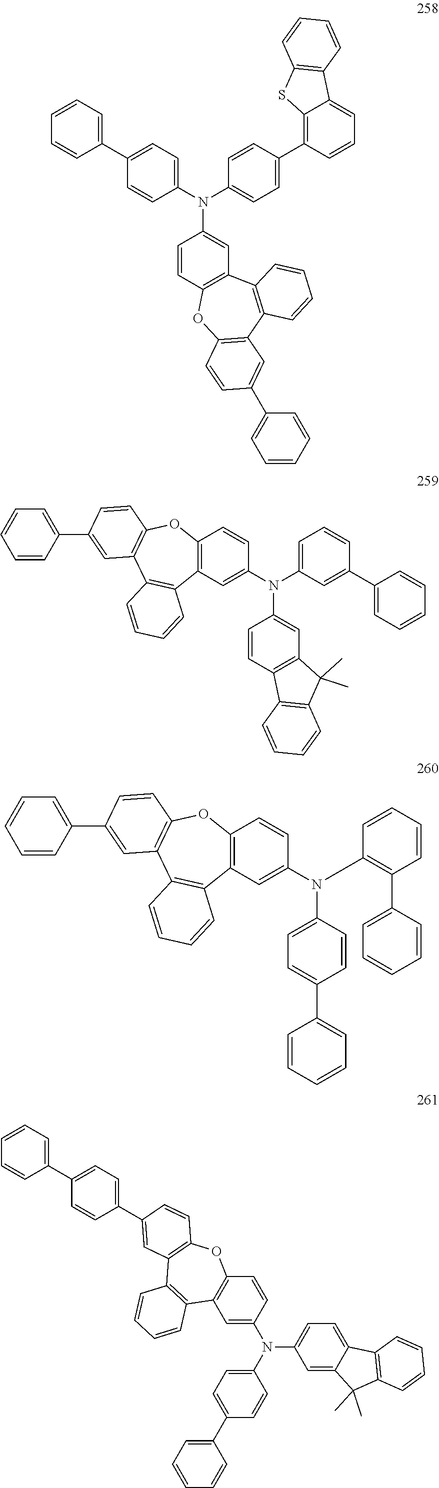 [SMILES: c1ccc(-c2ccc(N(c3ccc(-c4cccc5c4sc4ccccc45)cc3)c3ccc4c(c3)-c3ccccc3-c3cc(-c5ccccc5)ccc3O4)cc2)cc1 ; CC1(C)c2ccccc2-c2ccc(N(c3cccc(-c4ccccc4)c3)c3ccc4c(c3)-c3ccccc3-c3cc(-c5ccccc5)ccc3O4)cc21 ; c1ccc(-c2ccc(N(c3ccc4c(c3)-c3ccccc3-c3cc(-c5ccccc5)ccc3O4)c3ccccc3-c3ccccc3)cc2)cc1 ; CC1(C)c2ccccc2-c2ccc(N(c3ccc(-c4ccccc4)cc3)c3ccc4c(c3)-c3ccccc3-c3cc(-c5ccc(-c6ccccc6)cc5)ccc3O4)cc21]

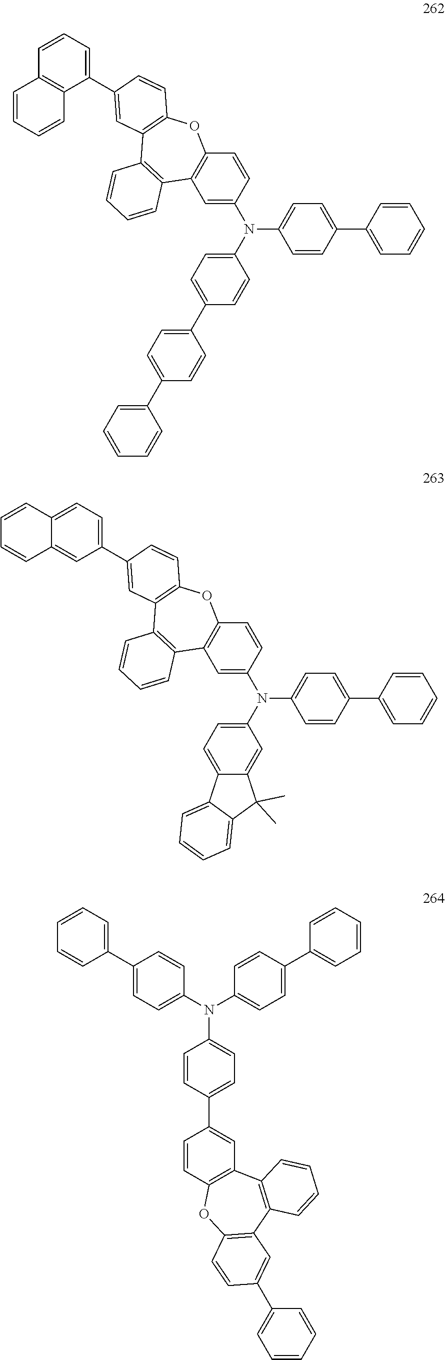 [SMILES: c1ccc(-c2ccc(-c3ccc(N(c4ccc(-c5ccccc5)cc4)c4ccc5c(c4)-c4ccccc4-c4cc(-c6cccc7ccccc67)ccc4O5)cc3)cc2)cc1 ; CC1(C)c2ccccc2-c2ccc(N(c3ccc(-c4ccccc4)cc3)c3ccc4c(c3)-c3ccccc3-c3cc(-c5ccc6ccccc6c5)ccc3O4)cc21 ; c1ccc(-c2ccc(N(c3ccc(-c4ccccc4)cc3)c3ccc(-c4ccc5c(c4)-c4ccccc4-c4cc(-c6ccccc6)ccc4O5)cc3)cc2)cc1]

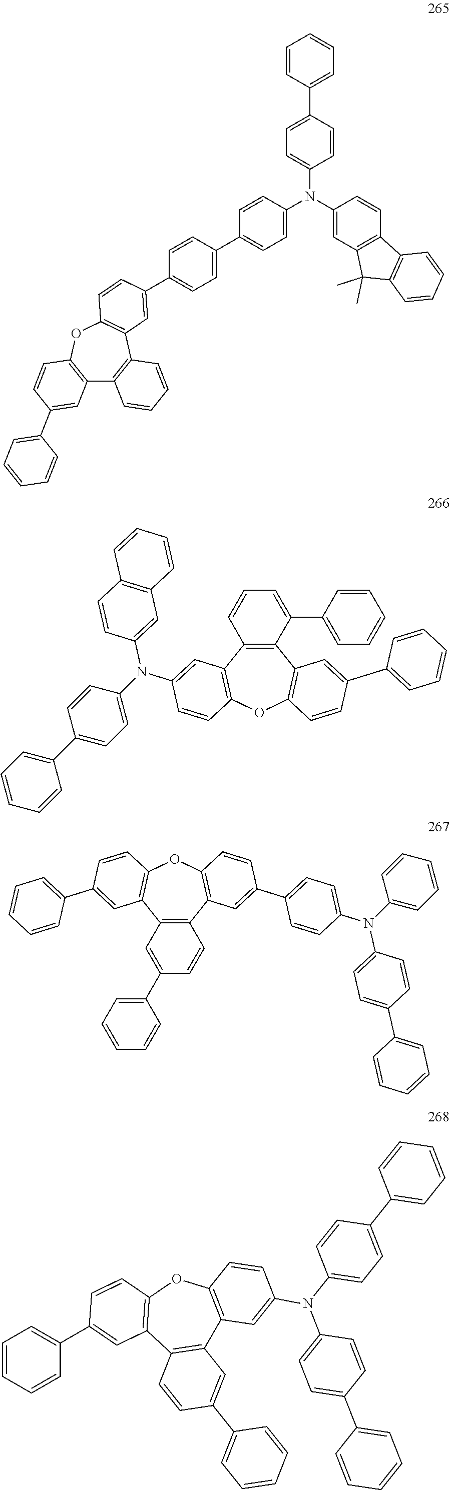 [SMILES: CC1(C)c2ccccc2-c2ccc(N(c3ccc(-c4ccccc4)cc3)c3ccc(-c4ccc(-c5ccc6c(c5)-c5ccccc5-c5cc(-c7ccccc7)ccc5O6)cc4)cc3)cc21 ; c1ccc(-c2ccc(N(c3ccc4c(c3)-c3cccc(-c5ccccc5)c3-c3cc(-c5ccccc5)ccc3O4)c3ccc4ccccc4c3)cc2)cc1 ; c1ccc(-c2ccc(N(c3ccccc3)c3ccc(-c4ccc5c(c4)-c4ccc(-c6ccccc6)cc4-c4cc(-c6ccccc6)ccc4O5)cc3)cc2)cc1 ; c1ccc(-c2ccc(N(c3ccc(-c4ccccc4)cc3)c3ccc4c(c3)-c3cc(-c5ccccc5)ccc3-c3cc(-c5ccccc5)ccc3O4)cc2)cc1]

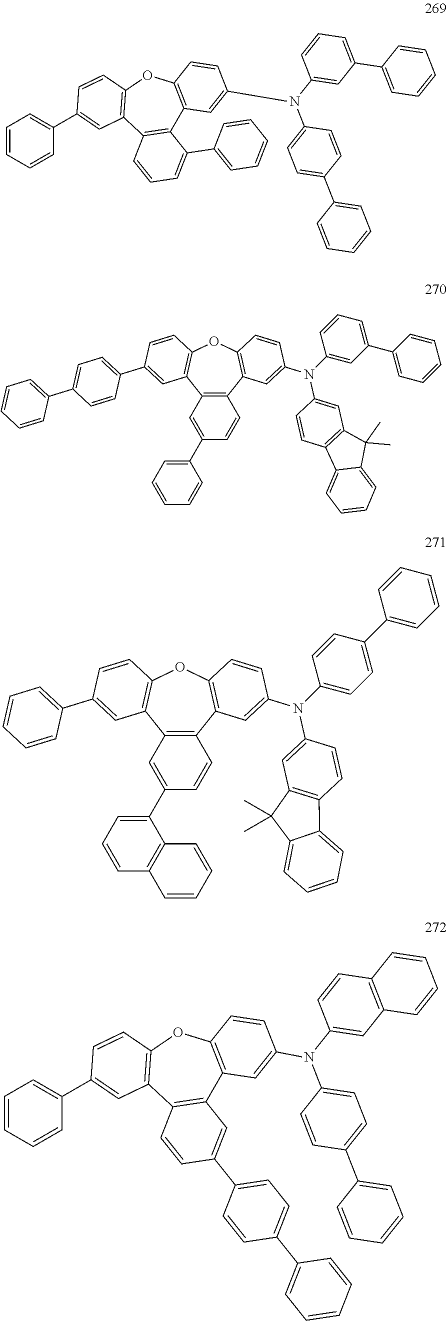 [SMILES: c1ccc(-c2ccc(N(c3cccc(-c4ccccc4)c3)c3ccc4c(c3)-c3c(-c5ccccc5)cccc3-c3cc(-c5ccccc5)ccc3O4)cc2)cc1 ; CC1(C)c2ccccc2-c2ccc(N(c3cccc(-c4ccccc4)c3)c3ccc4c(c3)-c3ccc(-c5ccccc5)cc3-c3cc(-c5ccc(-c6ccccc6)cc5)ccc3O4)cc21 ; CC1(C)c2ccccc2-c2ccc(N(c3ccc(-c4ccccc4)cc3)c3ccc4c(c3)-c3ccc(-c5cccc6ccccc56)cc3-c3cc(-c5ccccc5)ccc3O4)cc21 ; c1ccc(-c2ccc(-c3ccc4c(c3)-c3cc(N(c5ccc(-c6ccccc6)cc5)c5ccc6ccccc6c5)ccc3Oc3ccc(-c5ccccc5)cc3-4)cc2)cc1]

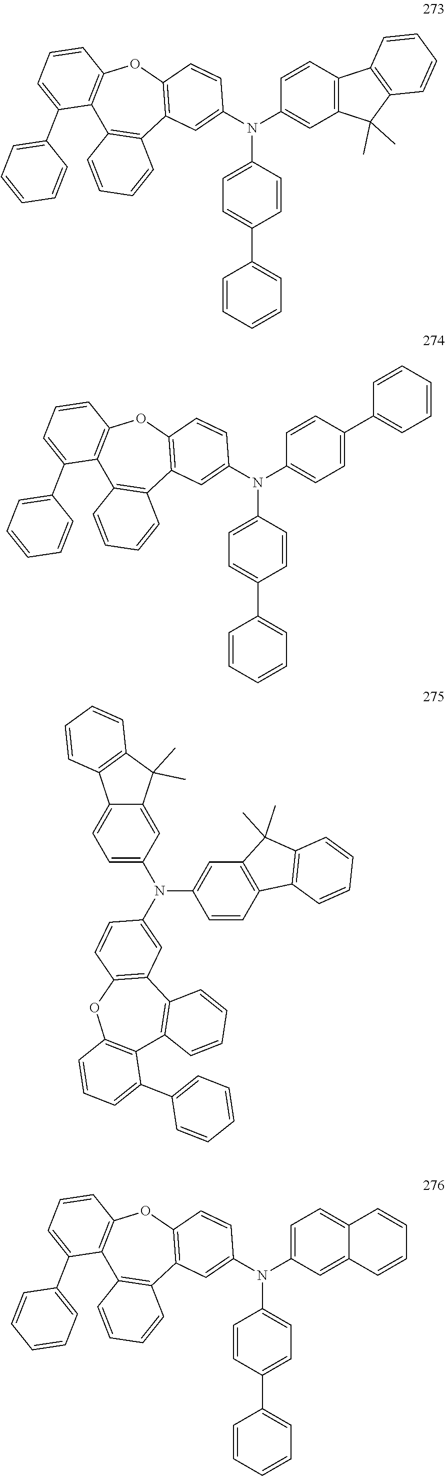 [SMILES: CC1(C)c2ccccc2-c2ccc(N(c3ccc(-c4ccccc4)cc3)c3ccc4c(c3)-c3ccccc3-c3c(cccc3-c3ccccc3)O4)cc21 ; c1ccc(-c2ccc(N(c3ccc(-c4ccccc4)cc3)c3ccc4c(c3)-c3ccccc3-c3c(cccc3-c3ccccc3)O4)cc2)cc1 ; CC1(C)c2ccccc2-c2ccc(N(c3ccc4c(c3)-c3ccccc3-c3c(cccc3-c3ccccc3)O4)c3ccc4c(c3)C(C)(C)c3ccccc3-4)cc21 ; c1ccc(-c2ccc(N(c3ccc4c(c3)-c3ccccc3-c3c(cccc3-c3ccccc3)O4)c3ccc4ccccc4c3)cc2)cc1]

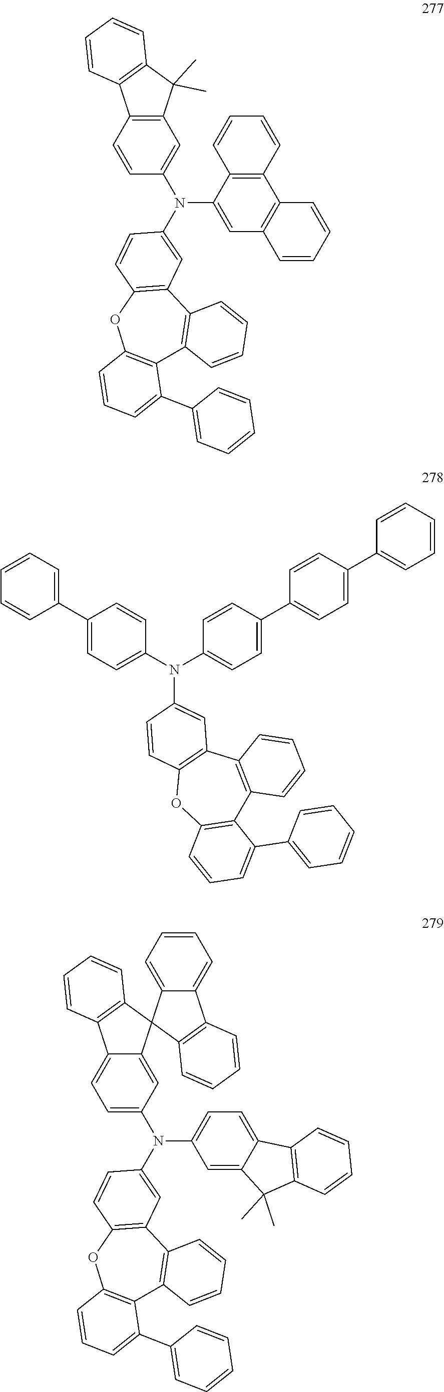 [SMILES: CC1(C)c2ccccc2-c2ccc(N(c3ccc4c(c3)-c3ccccc3-c3c(cccc3-c3ccccc3)O4)c3cc4ccccc4c4ccccc34)cc21 ; c1ccc(-c2ccc(-c3ccc(N(c4ccc(-c5ccccc5)cc4)c4ccc5c(c4)-c4ccccc4-c4c(cccc4-c4ccccc4)O5)cc3)cc2)cc1 ; CC1(C)c2ccccc2-c2ccc(N(c3ccc4c(c3)-c3ccccc3-c3c(cccc3-c3ccccc3)O4)c3ccc4c(c3)C3(c5ccccc5-c5ccccc53)c3ccccc3-4)cc21]

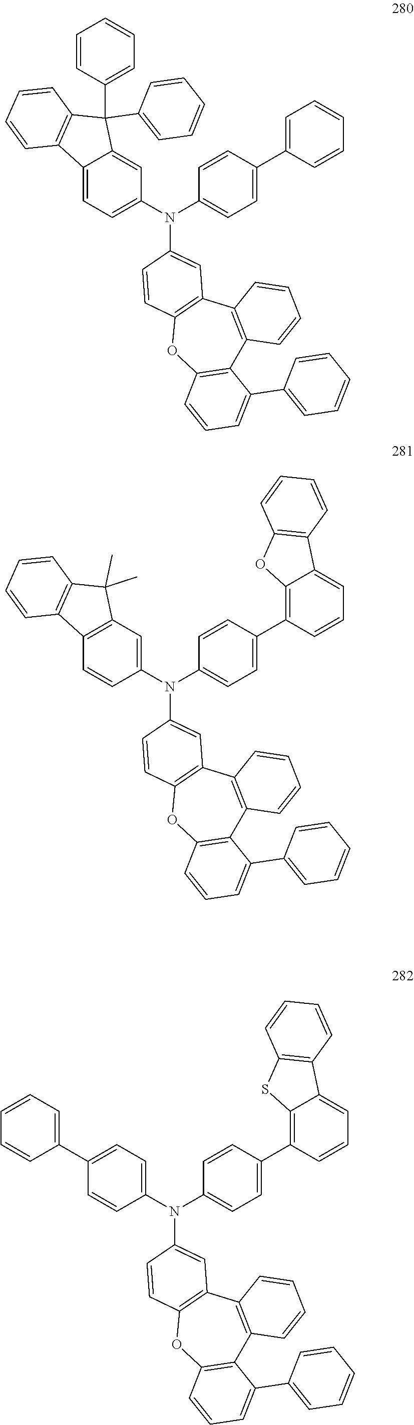 [SMILES: c1ccc(-c2ccc(N(c3ccc4c(c3)-c3ccccc3-c3c(cccc3-c3ccccc3)O4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)-c3ccccc3-c3c(cccc3-c3ccccc3)O4)cc21 ; c1ccc(-c2ccc(N(c3ccc(-c4cccc5c4sc4ccccc45)cc3)c3ccc4c(c3)-c3ccccc3-c3c(cccc3-c3ccccc3)O4)cc2)cc1]

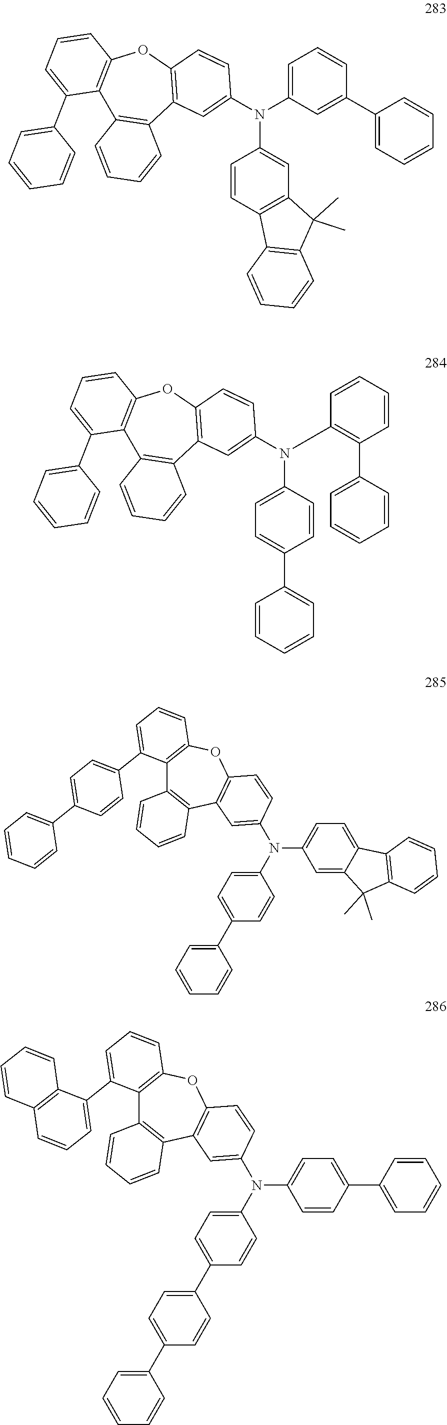 [SMILES: CC1(C)c2ccccc2-c2ccc(N(c3cccc(-c4ccccc4)c3)c3ccc4c(c3)-c3ccccc3-c3c(cccc3-c3ccccc3)O4)cc21 ; c1ccc(-c2ccc(N(c3ccc4c(c3)-c3ccccc3-c3c(cccc3-c3ccccc3)O4)c3ccccc3-c3ccccc3)cc2)cc1 ; CC1(C)c2ccccc2-c2ccc(N(c3ccc(-c4ccccc4)cc3)c3ccc4c(c3)-c3ccccc3-c3c(cccc3-c3ccc(-c5ccccc5)cc3)O4)cc21 ; c1ccc(-c2ccc(-c3ccc(N(c4ccc(-c5ccccc5)cc4)c4ccc5c(c4)-c4ccccc4-c4c(cccc4-c4cccc6ccccc46)O5)cc3)cc2)cc1]

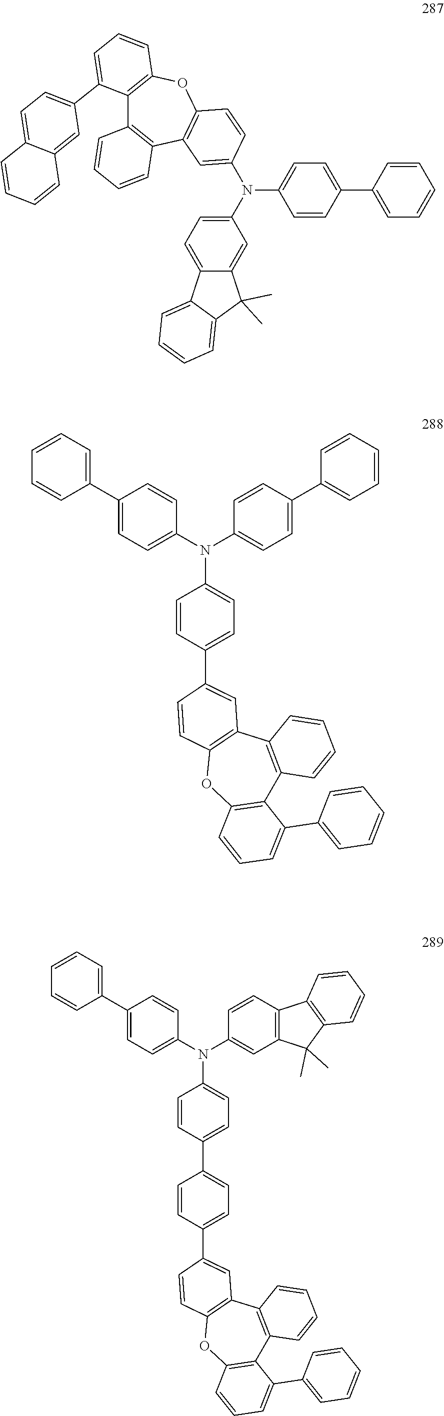 [SMILES: CC1(C)c2ccccc2-c2ccc(N(c3ccc(-c4ccccc4)cc3)c3ccc4c(c3)-c3ccccc3-c3c(cccc3-c3ccc5ccccc5c3)O4)cc21 ; c1ccc(-c2ccc(N(c3ccc(-c4ccccc4)cc3)c3ccc(-c4ccc5c(c4)-c4ccccc4-c4c(cccc4-c4ccccc4)O5)cc3)cc2)cc1 ; CC1(C)c2ccccc2-c2ccc(N(c3ccc(-c4ccccc4)cc3)c3ccc(-c4ccc(-c5ccc6c(c5)-c5ccccc5-c5c(cccc5-c5ccccc5)O6)cc4)cc3)cc21]

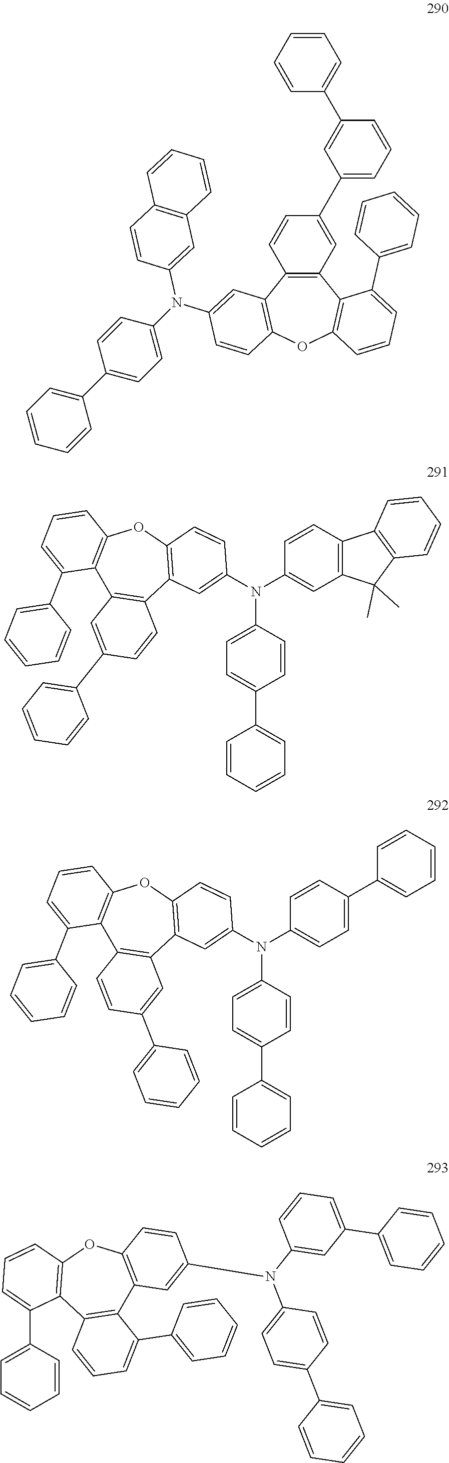 [SMILES: c1ccc(-c2ccc(N(c3ccc4c(c3)-c3ccc(-c5cccc(-c6ccccc6)c5)cc3-c3c(cccc3-c3ccccc3)O4)c3ccc4ccccc4c3)cc2)cc1 ; CC1(C)c2ccccc2-c2ccc(N(c3ccc(-c4ccccc4)cc3)c3ccc4c(c3)-c3ccc(-c5ccccc5)cc3-c3c(cccc3-c3ccccc3)O4)cc21 ; c1ccc(-c2ccc(N(c3ccc(-c4ccccc4)cc3)c3ccc4c(c3)-c3cc(-c5ccccc5)ccc3-c3c(cccc3-c3ccccc3)O4)cc2)cc1 ; c1ccc(-c2ccc(N(c3cccc(-c4ccccc4)c3)c3ccc4c(c3)-c3c(-c5ccccc5)cccc3-c3c(cccc3-c3ccccc3)O4)cc2)cc1]

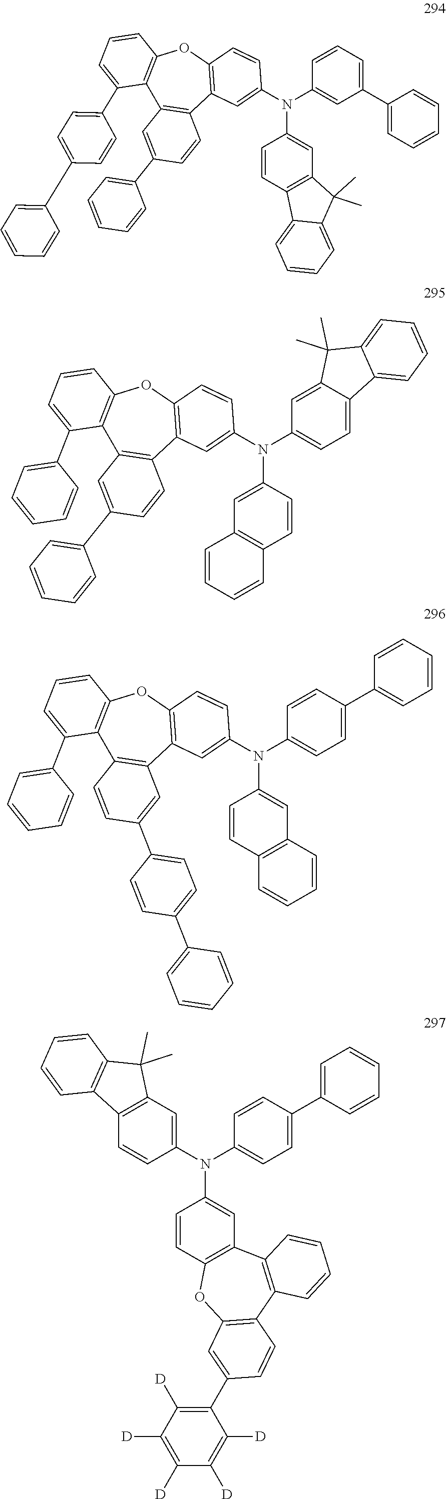

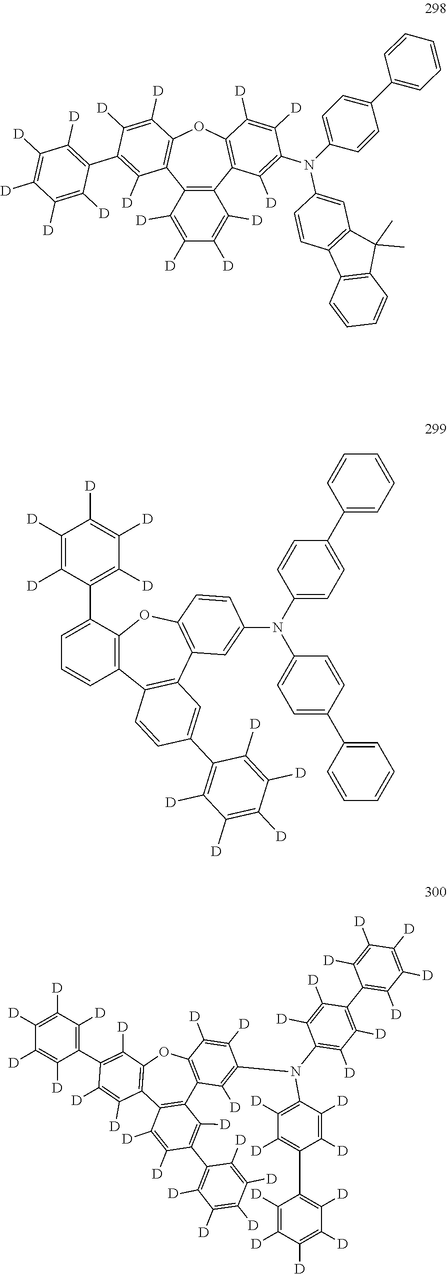

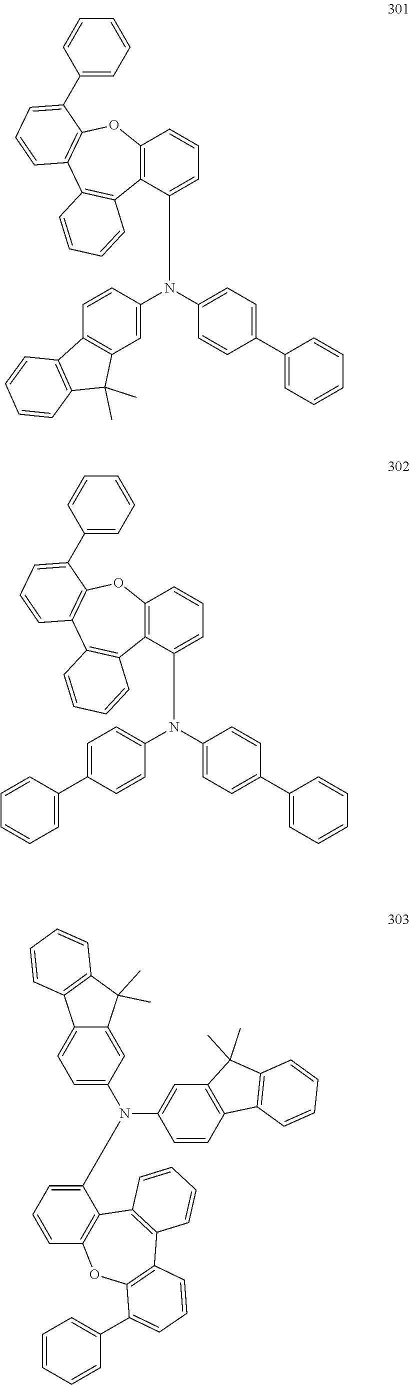 [SMILES: CC1(C)c2ccccc2-c2ccc(N(c3ccc(-c4ccccc4)cc3)c3cccc4c3-c3ccccc3-c3cccc(-c5ccccc5)c3O4)cc21 ; c1ccc(-c2ccc(N(c3ccc(-c4ccccc4)cc3)c3cccc4c3-c3ccccc3-c3cccc(-c5ccccc5)c3O4)cc2)cc1 ; CC1(C)c2ccccc2-c2ccc(N(c3ccc4c(c3)C(C)(C)c3ccccc3-4)c3cccc4c3-c3ccccc3-c3cccc(-c5ccccc5)c3O4)cc21]

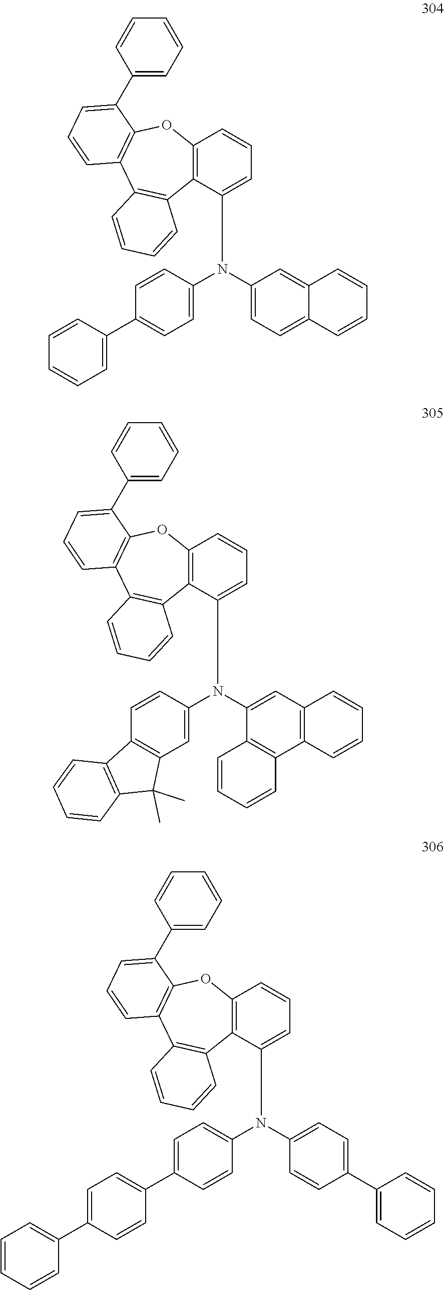 [SMILES: c1ccc(-c2ccc(N(c3ccc4ccccc4c3)c3cccc4c3-c3ccccc3-c3cccc(-c5ccccc5)c3O4)cc2)cc1 ; CC1(C)c2ccccc2-c2ccc(N(c3cccc4c3-c3ccccc3-c3cccc(-c5ccccc5)c3O4)c3cc4ccccc4c4ccccc34)cc21 ; c1ccc(-c2ccc(-c3ccc(N(c4ccc(-c5ccccc5)cc4)c4cccc5c4-c4ccccc4-c4cccc(-c6ccccc6)c4O5)cc3)cc2)cc1]

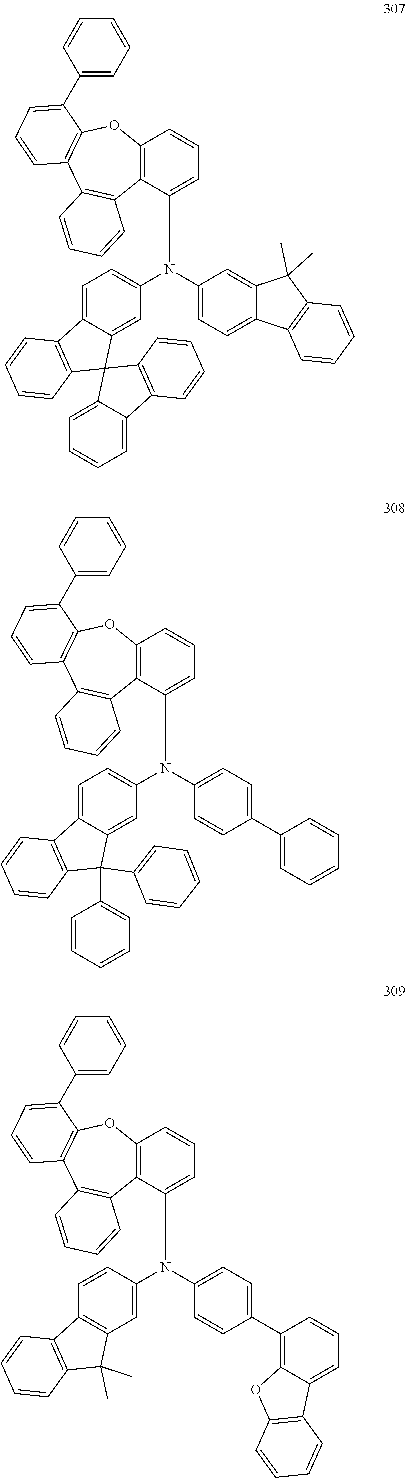 [SMILES: CC1(C)c2ccccc2-c2ccc(N(c3ccc4c(c3)C3(c5ccccc5-c5ccccc53)c3ccccc3-4)c3cccc4c3-c3ccccc3-c3cccc(-c5ccccc5)c3O4)cc21 ; c1ccc(-c2ccc(N(c3ccc4c(c3)C(c3ccccc3)(c3ccccc3)c3ccccc3-4)c3cccc4c3-c3ccccc3-c3cccc(-c5ccccc5)c3O4)cc2)cc1 ; CC1(C)c2ccccc2-c2ccc(N(c3ccc(-c4cccc5c4oc4ccccc45)cc3)c3cccc4c3-c3ccccc3-c3cccc(-c5ccccc5)c3O4)cc21]

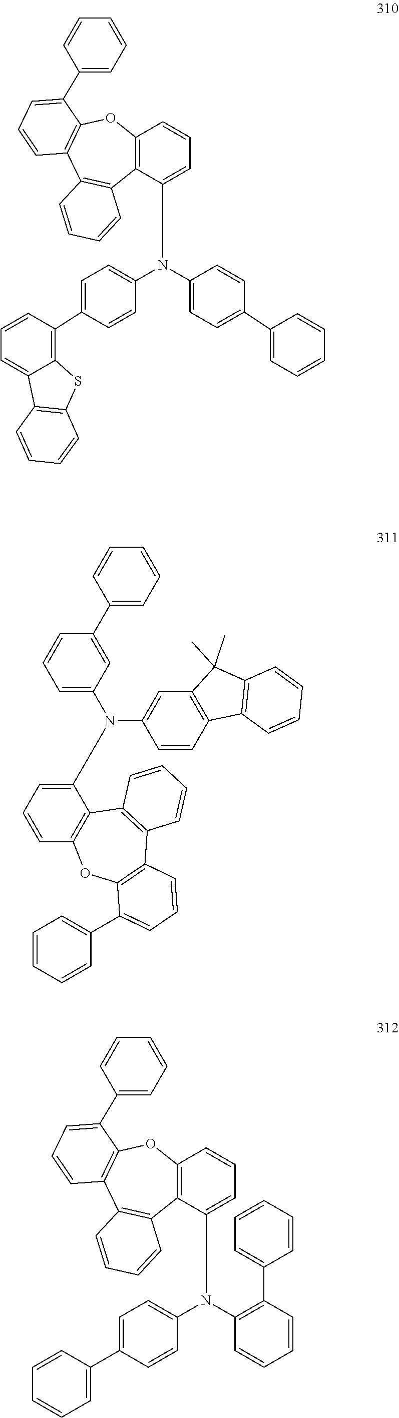 [SMILES: c1ccc(-c2ccc(N(c3ccc(-c4cccc5c4sc4ccccc45)cc3)c3cccc4c3-c3ccccc3-c3cccc(-c5ccccc5)c3O4)cc2)cc1 ; CC1(C)c2ccccc2-c2ccc(N(c3cccc(-c4ccccc4)c3)c3cccc4c3-c3ccccc3-c3cccc(-c5ccccc5)c3O4)cc21 ; c1ccc(-c2ccc(N(c3ccccc3-c3ccccc3)c3cccc4c3-c3ccccc3-c3cccc(-c5ccccc5)c3O4)cc2)cc1]

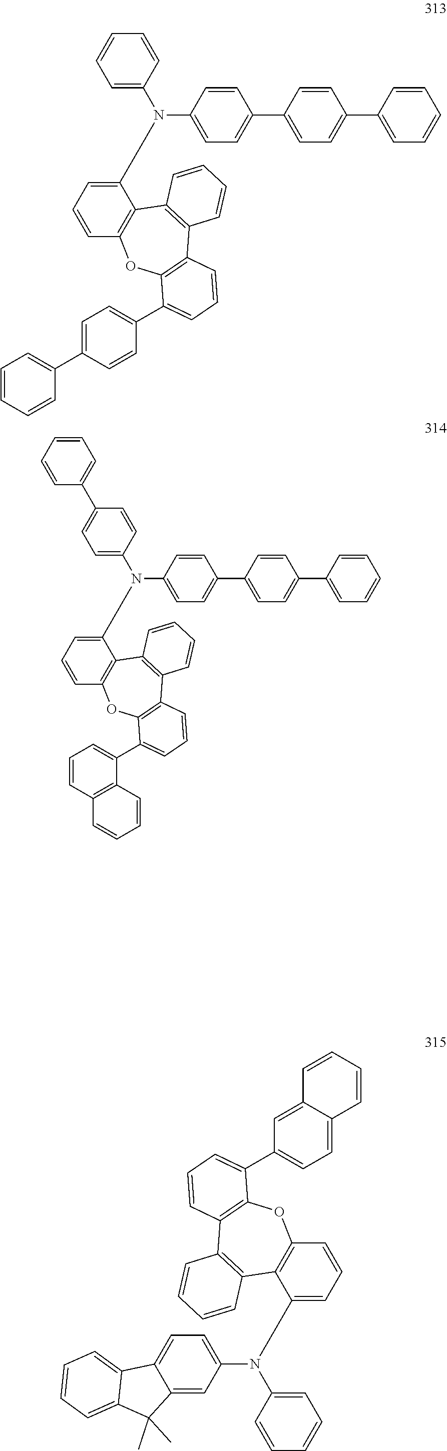

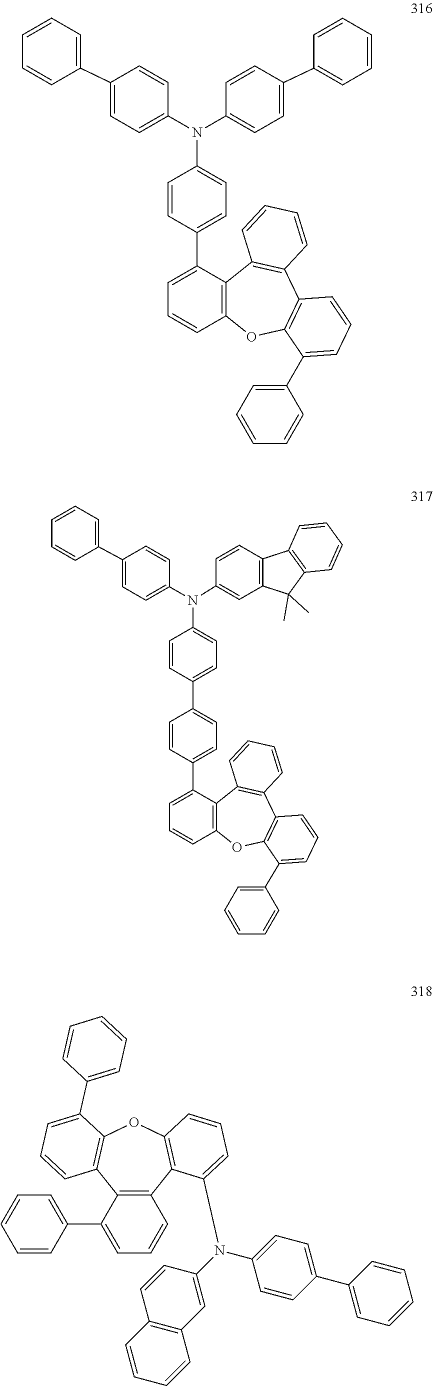 [SMILES: c1ccc(-c2ccc(N(c3ccc(-c4ccccc4)cc3)c3ccc(-c4cccc5c4-c4ccccc4-c4cccc(-c6ccccc6)c4O5)cc3)cc2)cc1 ; CC1(C)c2ccccc2-c2ccc(N(c3ccc(-c4ccccc4)cc3)c3ccc(-c4ccc(-c5cccc6c5-c5ccccc5-c5cccc(-c7ccccc7)c5O6)cc4)cc3)cc21 ; c1ccc(-c2ccc(N(c3ccc4ccccc4c3)c3cccc4c3-c3cccc(-c5ccccc5)c3-c3cccc(-c5ccccc5)c3O4)cc2)cc1]

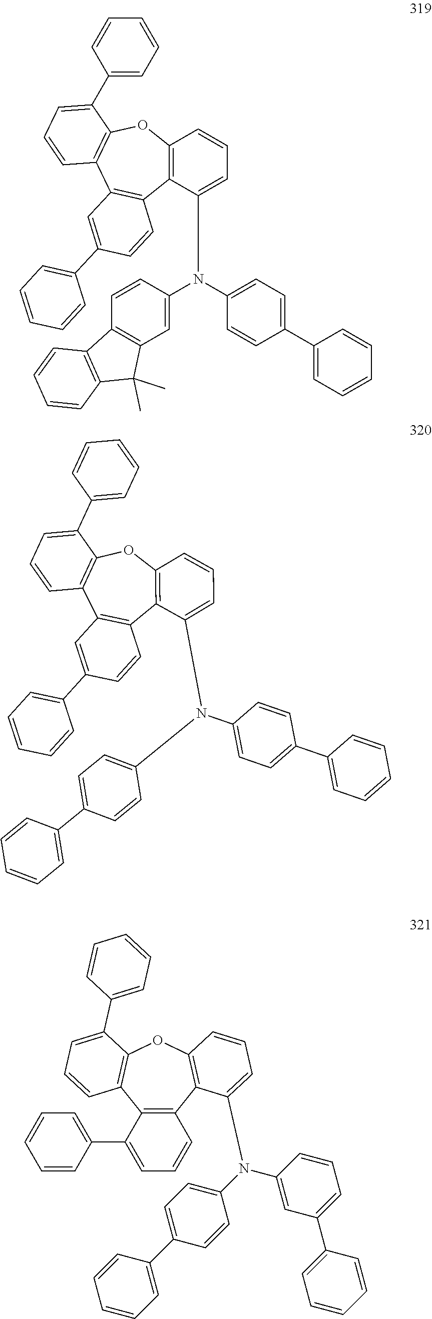

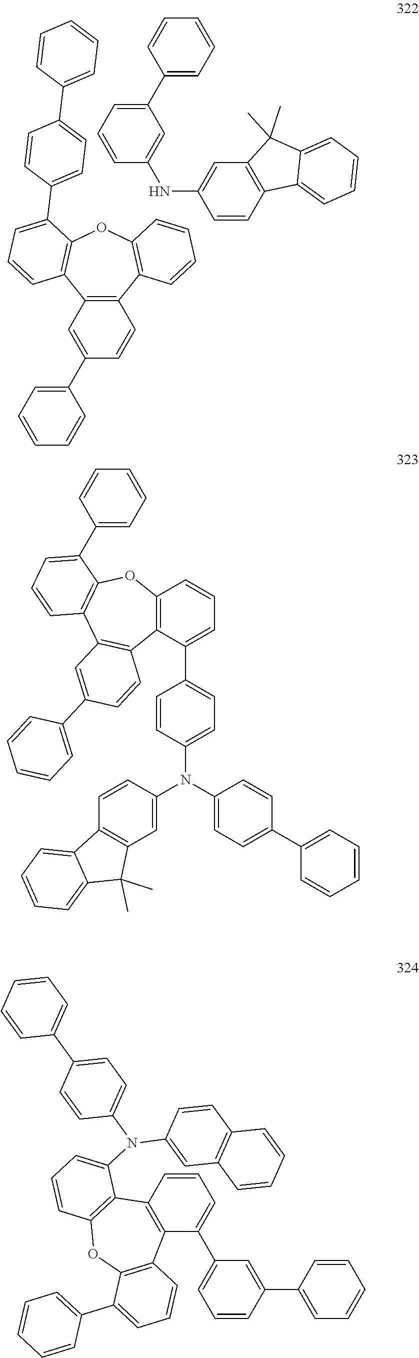

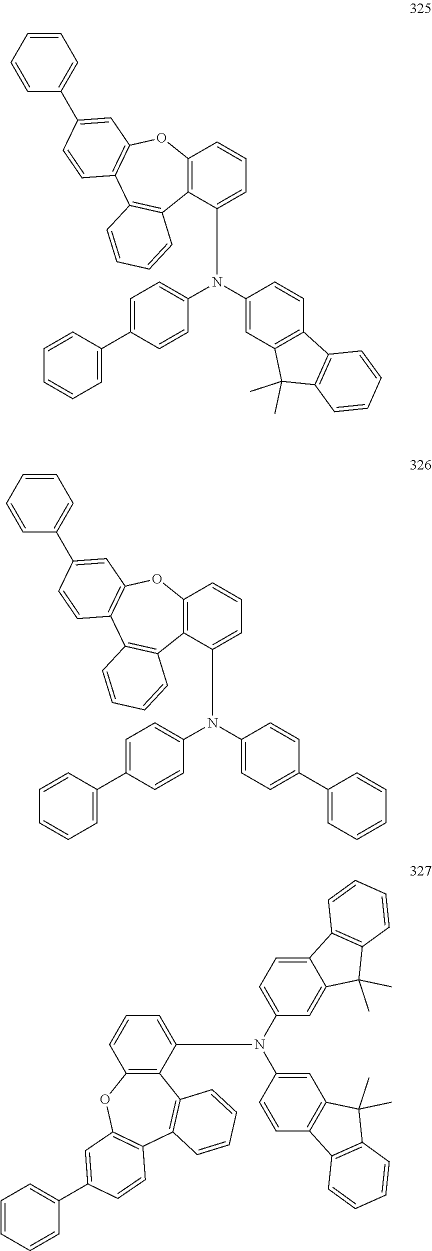

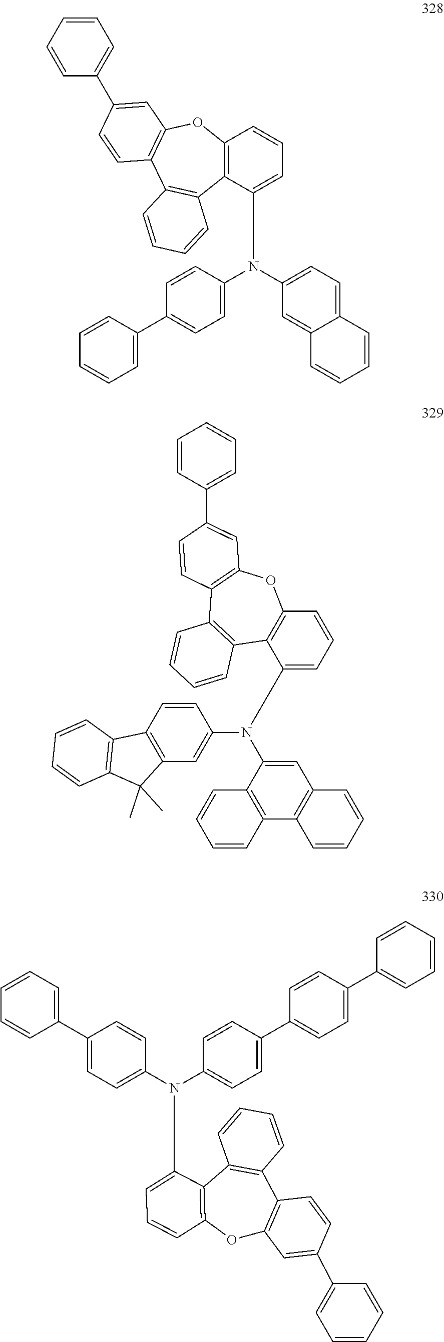 [SMILES: c1ccc(-c2ccc(N(c3ccc4ccccc4c3)c3cccc4c3-c3ccccc3-c3ccc(-c5ccccc5)cc3O4)cc2)cc1 ; CC1(C)c2ccccc2-c2ccc(N(c3cccc4c3-c3ccccc3-c3ccc(-c5ccccc5)cc3O4)c3cc4ccccc4c4ccccc34)cc21 ; c1ccc(-c2ccc(-c3ccc(N(c4ccc(-c5ccccc5)cc4)c4cccc5c4-c4ccccc4-c4ccc(-c6ccccc6)cc4O5)cc3)cc2)cc1]

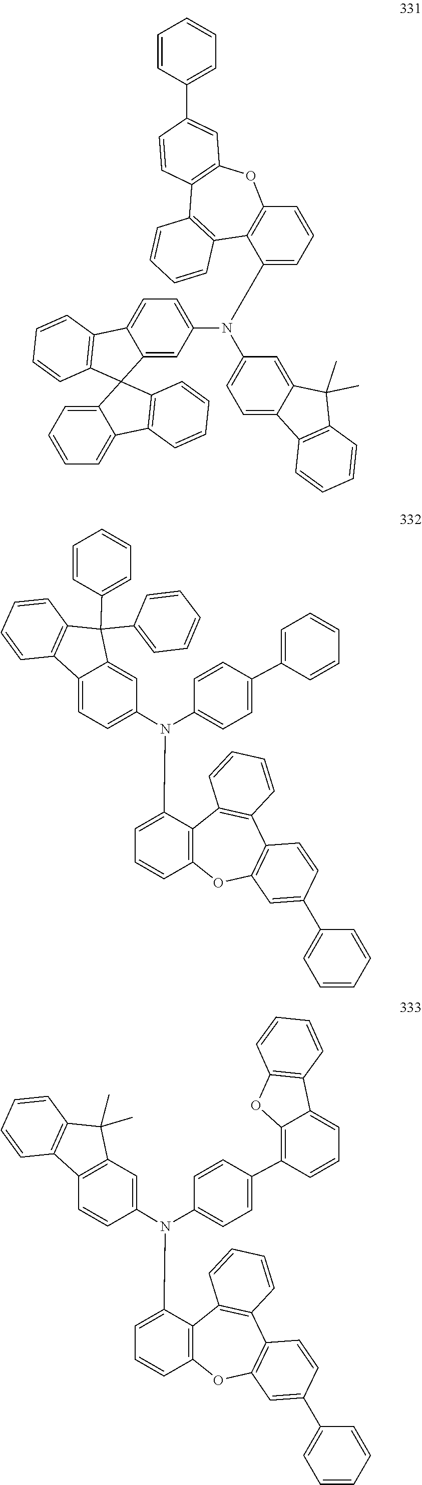

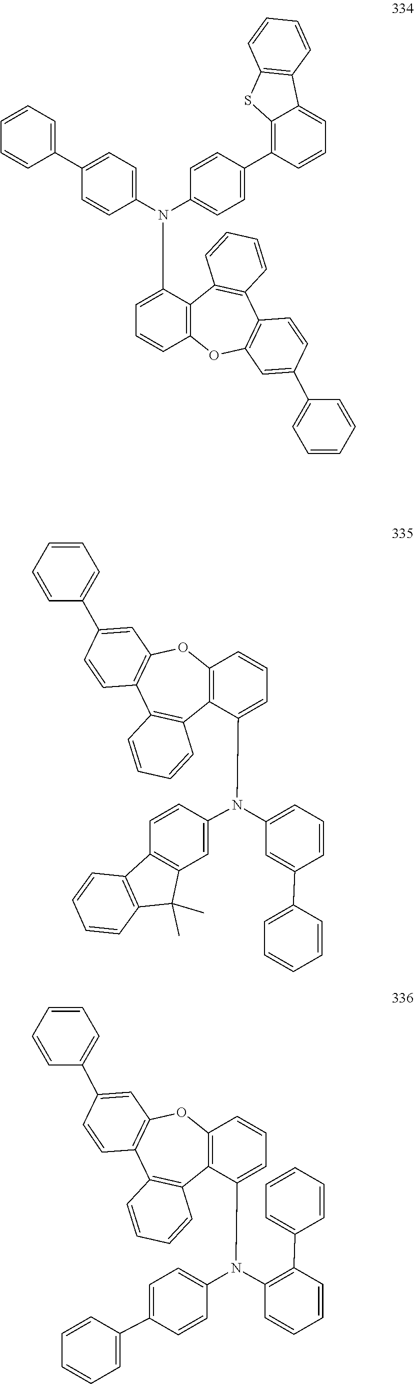 [SMILES: c1ccc(-c2ccc(N(c3ccc(-c4cccc5c4sc4ccccc45)cc3)c3cccc4c3-c3ccccc3-c3ccc(-c5ccccc5)cc3O4)cc2)cc1 ; CC1(C)c2ccccc2-c2ccc(N(c3cccc(-c4ccccc4)c3)c3cccc4c3-c3ccccc3-c3ccc(-c5ccccc5)cc3O4)cc21 ; c1ccc(-c2ccc(N(c3ccccc3-c3ccccc3)c3cccc4c3-c3ccccc3-c3ccc(-c5ccccc5)cc3O4)cc2)cc1]

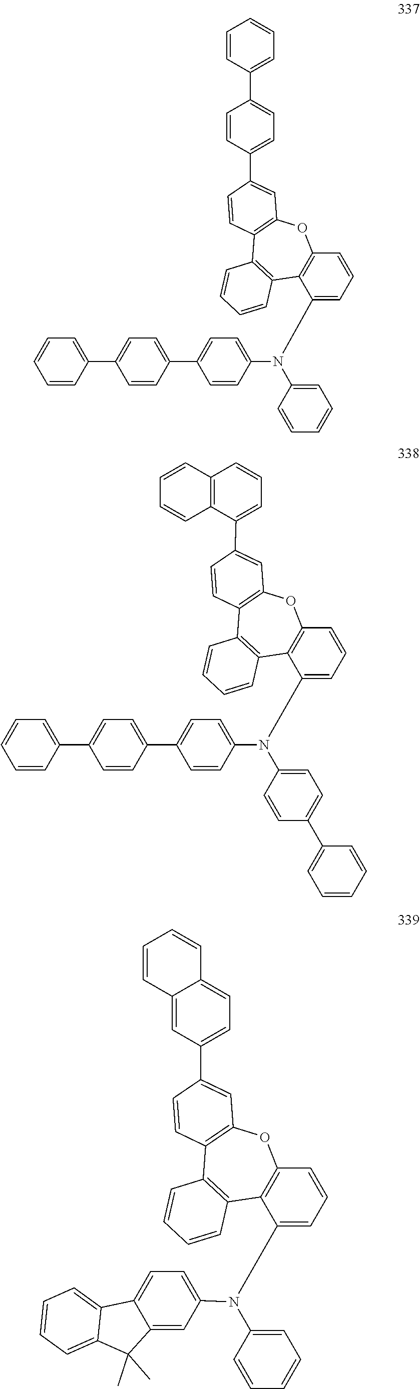 [SMILES: c1ccc(-c2ccc(-c3ccc(N(c4ccccc4)c4cccc5c4-c4ccccc4-c4ccc(-c6ccc(-c7ccccc7)cc6)cc4O5)cc3)cc2)cc1 ; c1ccc(-c2ccc(-c3ccc(N(c4ccc(-c5ccccc5)cc4)c4cccc5c4-c4ccccc4-c4ccc(-c6cccc7ccccc67)cc4O5)cc3)cc2)cc1 ; CC1(C)c2ccccc2-c2ccc(N(c3ccccc3)c3cccc4c3-c3ccccc3-c3ccc(-c5ccc6ccccc6c5)cc3O4)cc21]

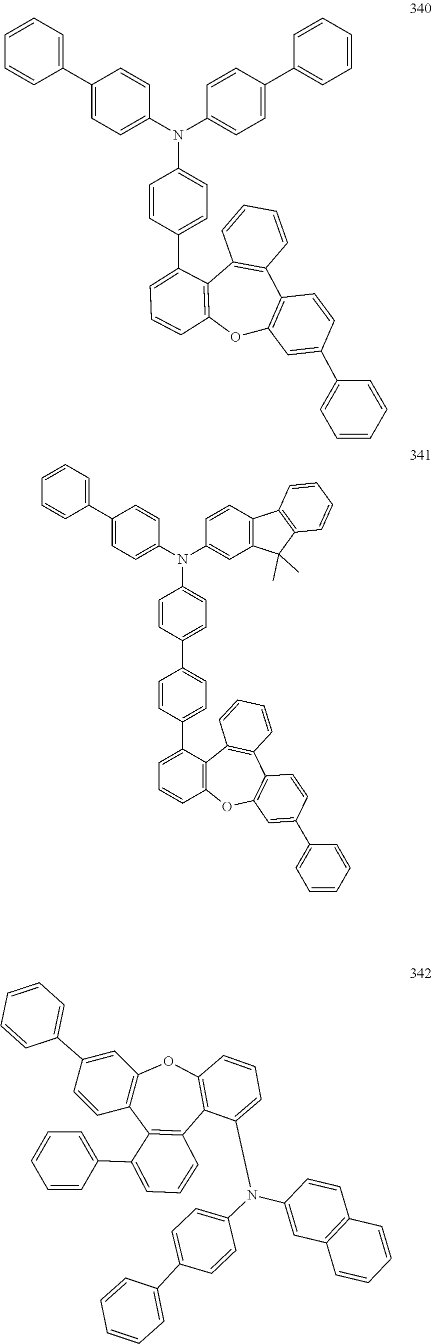 [SMILES: c1ccc(-c2ccc(N(c3ccc(-c4ccccc4)cc3)c3ccc(-c4cccc5c4-c4ccccc4-c4ccc(-c6ccccc6)cc4O5)cc3)cc2)cc1 ; CC1(C)c2ccccc2-c2ccc(N(c3ccc(-c4ccccc4)cc3)c3ccc(-c4ccc(-c5cccc6c5-c5ccccc5-c5ccc(-c7ccccc7)cc5O6)cc4)cc3)cc21 ; c1ccc(-c2ccc(N(c3ccc4ccccc4c3)c3cccc4c3-c3cccc(-c5ccccc5)c3-c3ccc(-c5ccccc5)cc3O4)cc2)cc1]

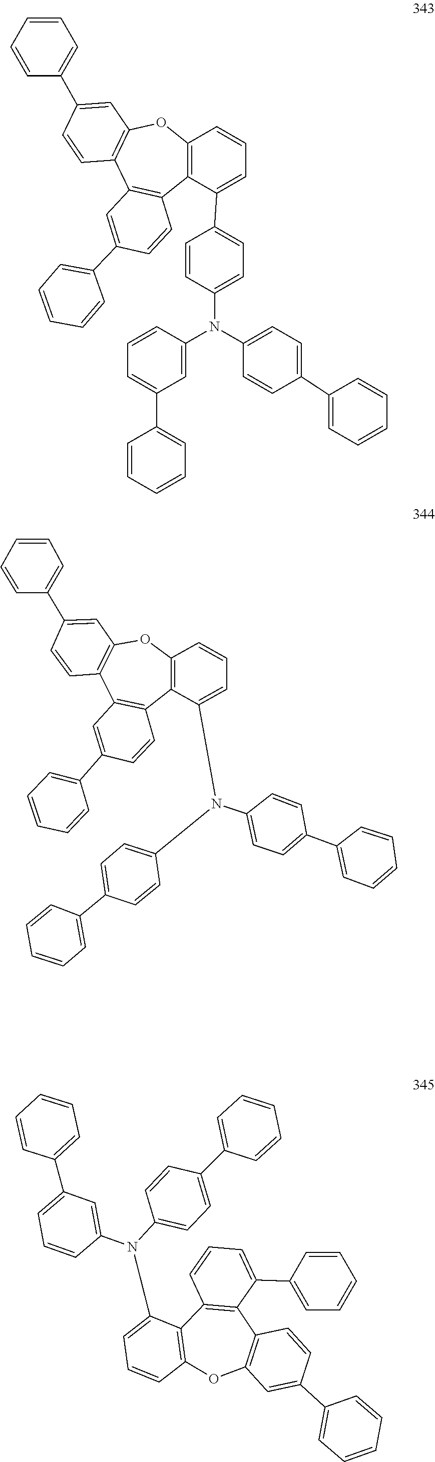 [SMILES: c1ccc(-c2ccc(N(c3ccc(-c4cccc5c4-c4ccc(-c6ccccc6)cc4-c4ccc(-c6ccccc6)cc4O5)cc3)c3cccc(-c4ccccc4)c3)cc2)cc1 ; c1ccc(-c2ccc(N(c3ccc(-c4ccccc4)cc3)c3cccc4c3-c3ccc(-c5ccccc5)cc3-c3ccc(-c5ccccc5)cc3O4)cc2)cc1 ; c1ccc(-c2ccc(N(c3cccc(-c4ccccc4)c3)c3cccc4c3-c3cccc(-c5ccccc5)c3-c3ccc(-c5ccccc5)cc3O4)cc2)cc1]

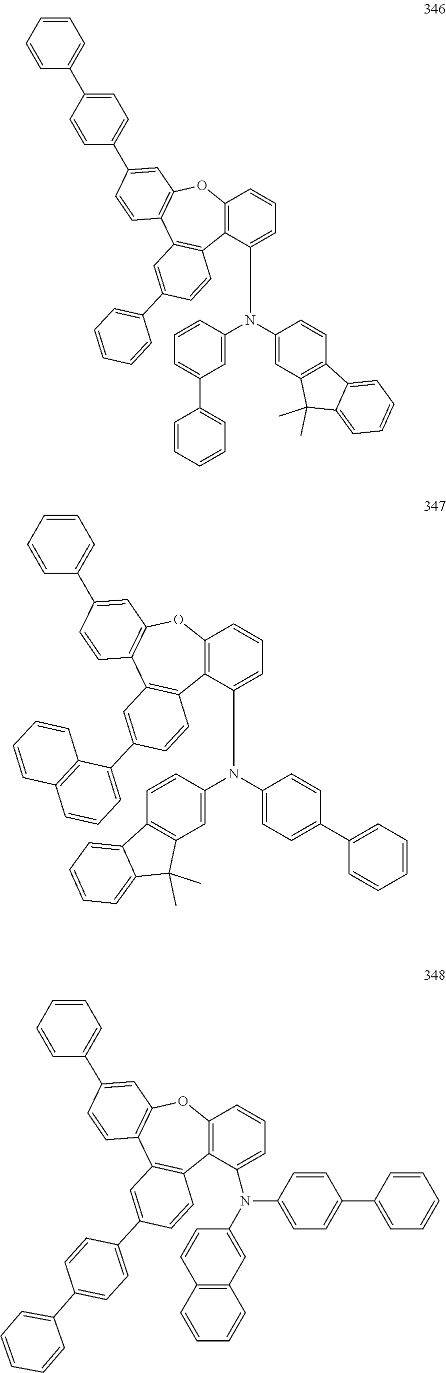 [SMILES: CC1(C)c2ccccc2-c2ccc(N(c3cccc(-c4ccccc4)c3)c3cccc4c3-c3ccc(-c5ccccc5)cc3-c3ccc(-c5ccc(-c6ccccc6)cc5)cc3O4)cc21 ; CC1(C)c2ccccc2-c2ccc(N(c3ccc(-c4ccccc4)cc3)c3cccc4c3-c3ccc(-c5cccc6ccccc56)cc3-c3ccc(-c5ccccc5)cc3O4)cc21 ; c1ccc(-c2ccc(-c3ccc4c(c3)-c3ccc(-c5ccccc5)cc3Oc3cccc(N(c5ccc(-c6ccccc6)cc5)c5ccc6ccccc6c5)c3-4)cc2)cc1]

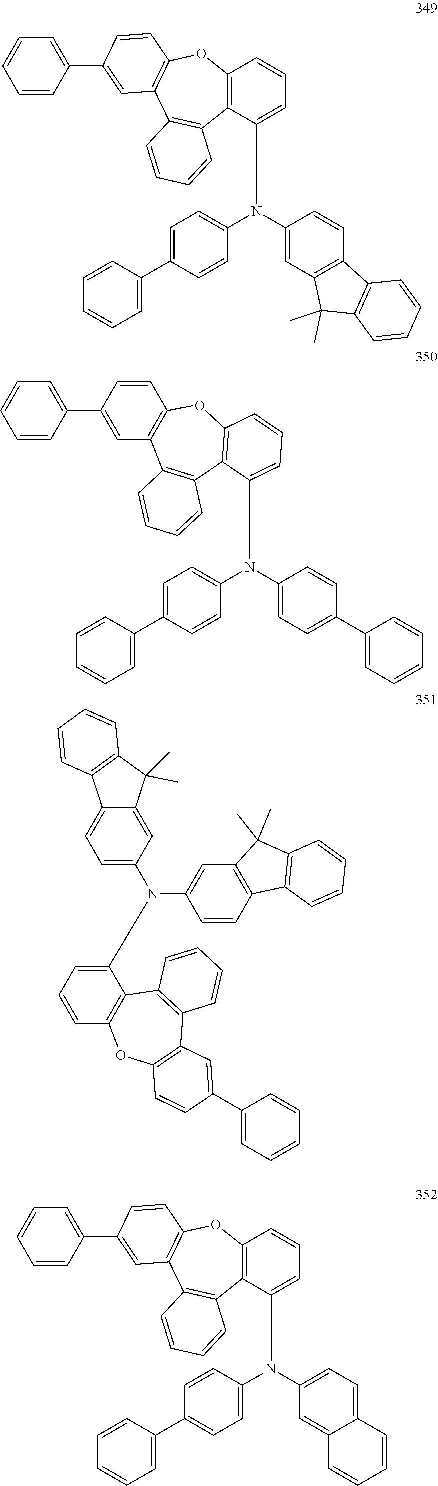

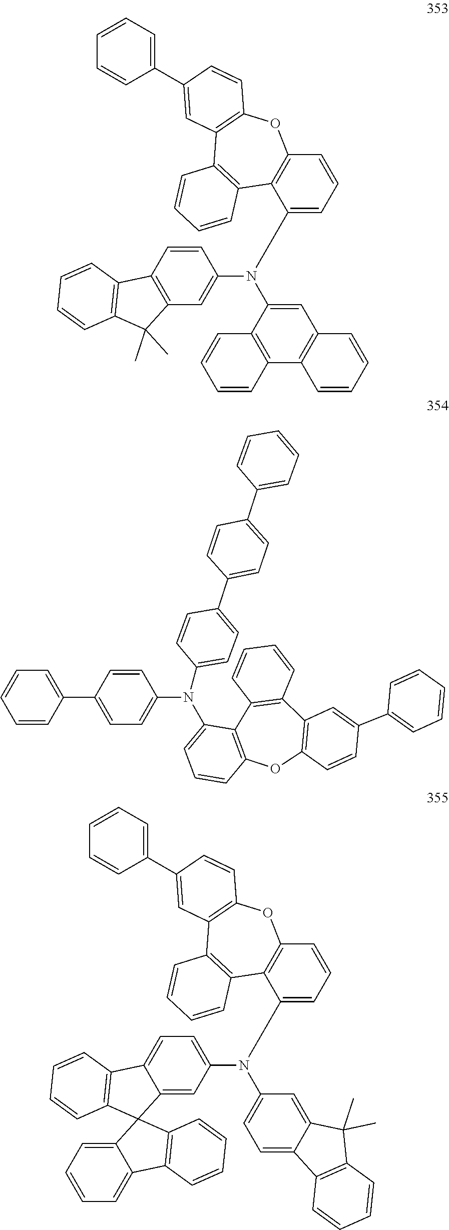

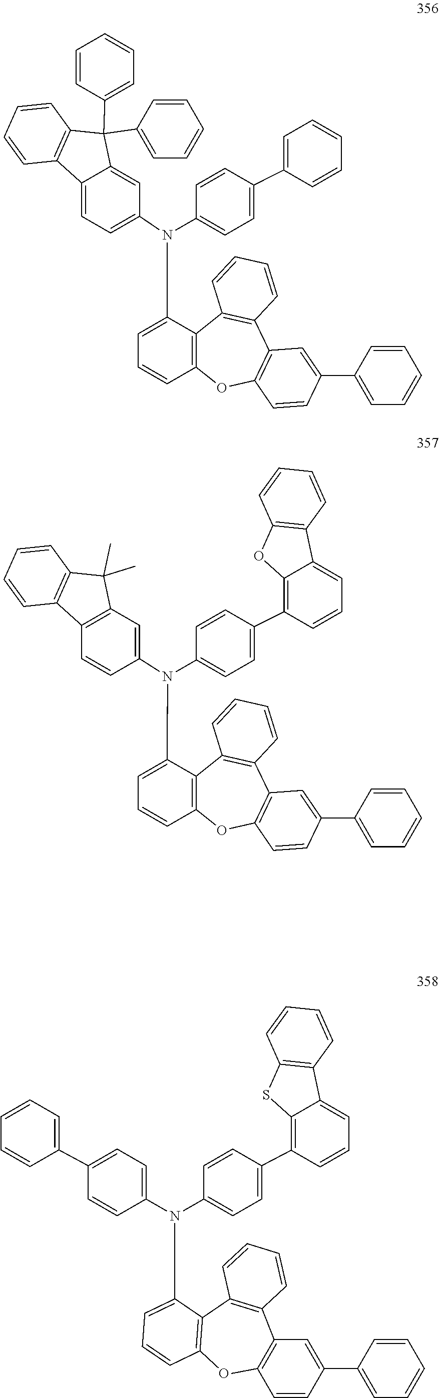 [SMILES: c1ccc(-c2ccc(N(c3ccc4c(c3)C(c3ccccc3)(c3ccccc3)c3ccccc3-4)c3cccc4c3-c3ccccc3-c3cc(-c5ccccc5)ccc3O4)cc2)cc1 ; CC1(C)c2ccccc2-c2ccc(N(c3ccc(-c4cccc5c4oc4ccccc45)cc3)c3cccc4c3-c3ccccc3-c3cc(-c5ccccc5)ccc3O4)cc21 ; c1ccc(-c2ccc(N(c3ccc(-c4cccc5c4sc4ccccc45)cc3)c3cccc4c3-c3ccccc3-c3cc(-c5ccccc5)ccc3O4)cc2)cc1]

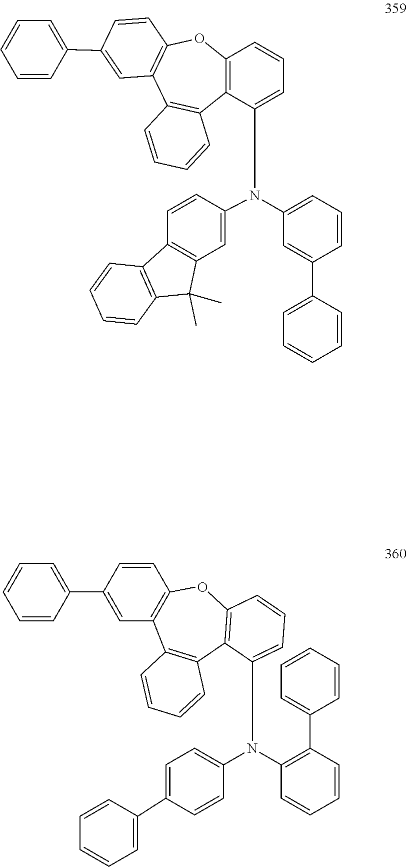 [SMILES: CC1(C)c2ccccc2-c2ccc(N(c3cccc(-c4ccccc4)c3)c3cccc4c3-c3ccccc3-c3cc(-c5ccccc5)ccc3O4)cc21 ; c1ccc(-c2ccc(N(c3ccccc3-c3ccccc3)c3cccc4c3-c3ccccc3-c3cc(-c5ccccc5)ccc3O4)cc2)cc1]

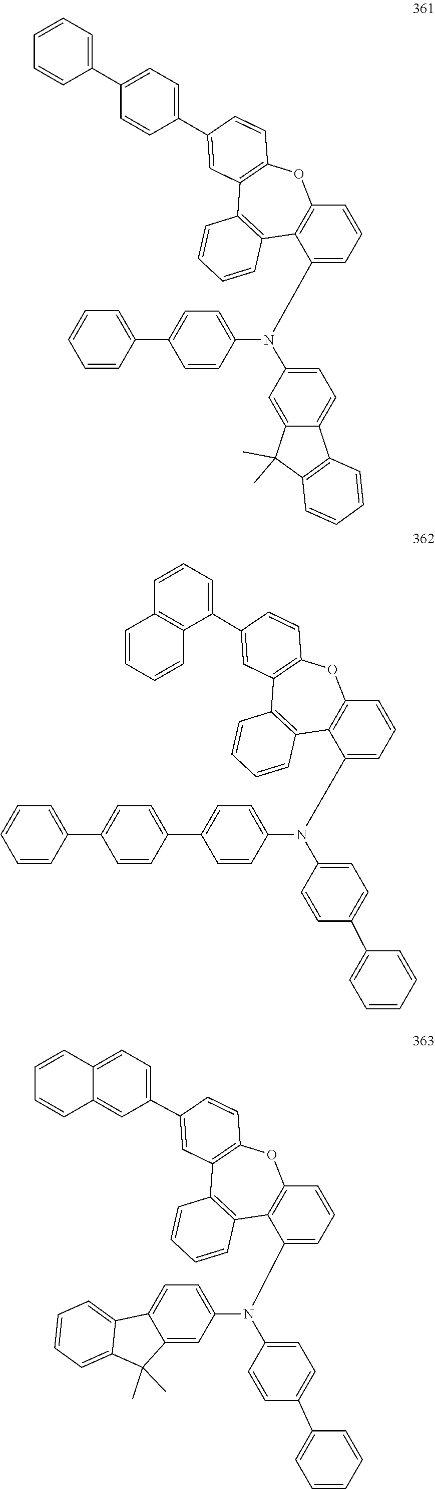 [SMILES: CC1(C)c2ccccc2-c2ccc(N(c3ccc(-c4ccccc4)cc3)c3cccc4c3-c3ccccc3-c3cc(-c5ccc(-c6ccccc6)cc5)ccc3O4)cc21 ; c1ccc(-c2ccc(-c3ccc(N(c4ccc(-c5ccccc5)cc4)c4cccc5c4-c4ccccc4-c4cc(-c6cccc7ccccc67)ccc4O5)cc3)cc2)cc1 ; CC1(C)c2ccccc2-c2ccc(N(c3ccc(-c4ccccc4)cc3)c3cccc4c3-c3ccccc3-c3cc(-c5ccc6ccccc6c5)ccc3O4)cc21]

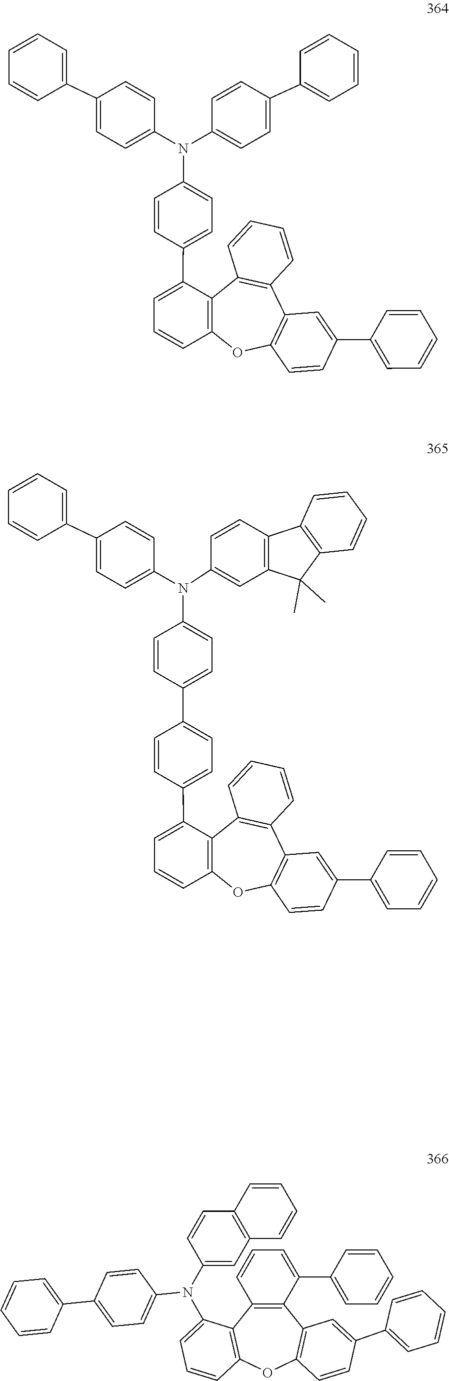 [SMILES: c1ccc(-c2ccc(N(c3ccc(-c4ccccc4)cc3)c3ccc(-c4cccc5c4-c4ccccc4-c4cc(-c6ccccc6)ccc4O5)cc3)cc2)cc1 ; CC1(C)c2ccccc2-c2ccc(N(c3ccc(-c4ccccc4)cc3)c3ccc(-c4ccc(-c5cccc6c5-c5ccccc5-c5cc(-c7ccccc7)ccc5O6)cc4)cc3)cc21 ; c1ccc(-c2ccc(N(c3ccc4ccccc4c3)c3cccc4c3-c3cccc(-c5ccccc5)c3-c3cc(-c5ccccc5)ccc3O4)cc2)cc1]

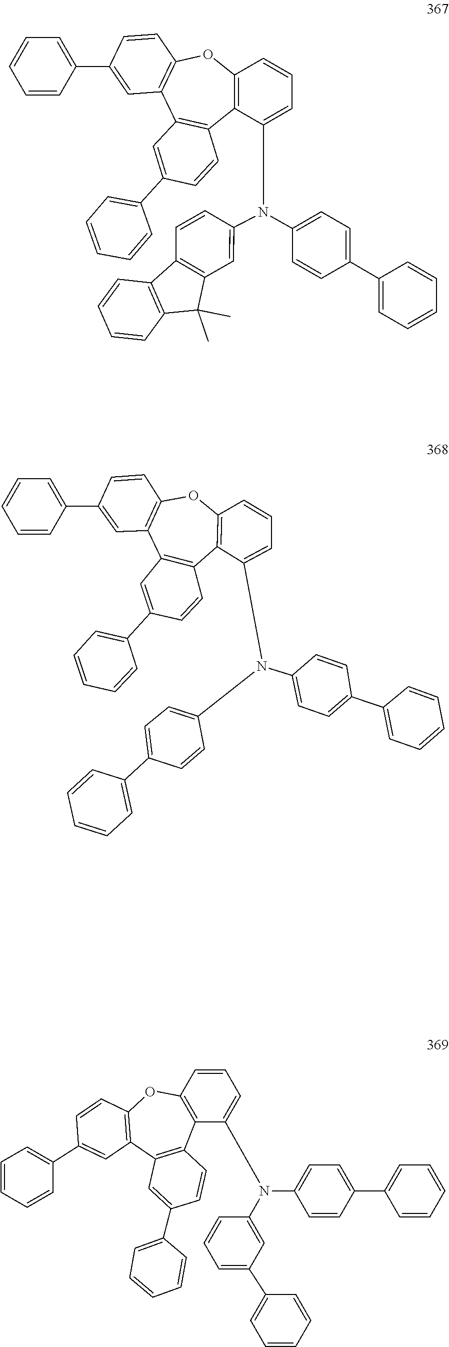 [SMILES: CC1(C)c2ccccc2-c2ccc(N(c3ccc(-c4ccccc4)cc3)c3cccc4c3-c3ccc(-c5ccccc5)cc3-c3cc(-c5ccccc5)ccc3O4)cc21 ; c1ccc(-c2ccc(N(c3ccc(-c4ccccc4)cc3)c3cccc4c3-c3ccc(-c5ccccc5)cc3-c3cc(-c5ccccc5)ccc3O4)cc2)cc1 ; c1ccc(-c2ccc(N(c3cccc(-c4ccccc4)c3)c3cccc4c3-c3ccc(-c5ccccc5)cc3-c3cc(-c5ccccc5)ccc3O4)cc2)cc1]

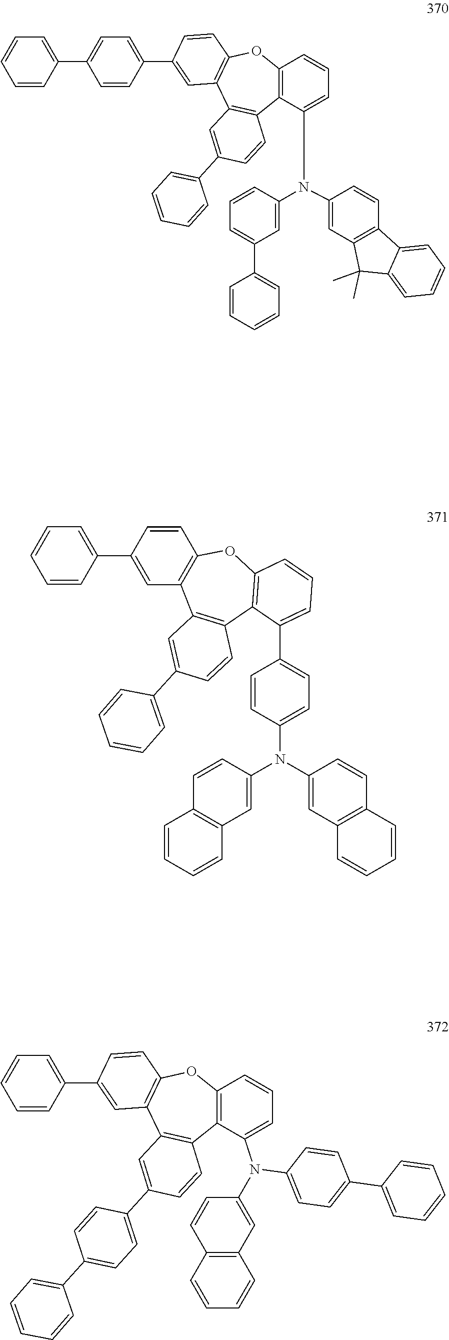 [SMILES: CC1(C)c2ccccc2-c2ccc(N(c3cccc(-c4ccccc4)c3)c3cccc4c3-c3ccc(-c5ccccc5)cc3-c3cc(-c5ccc(-c6ccccc6)cc5)ccc3O4)cc21 ; c1ccc(-c2ccc3c(c2)-c2cc(-c4ccccc4)ccc2-c2c(cccc2-c2ccc(N(c4ccc5ccccc5c4)c4ccc5ccccc5c4)cc2)O3)cc1 ; c1ccc(-c2ccc(-c3ccc4c(c3)-c3cc(-c5ccccc5)ccc3Oc3cccc(N(c5ccc(-c6ccccc6)cc5)c5ccc6ccccc6c5)c3-4)cc2)cc1]

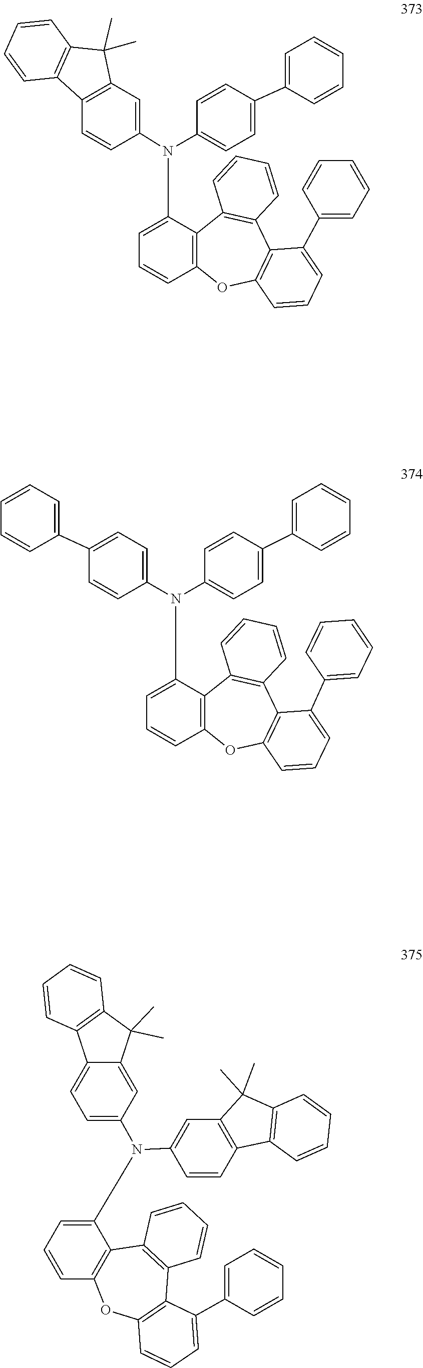 [SMILES: CC1(C)c2ccccc2-c2ccc(N(c3ccc(-c4ccccc4)cc3)c3cccc4c3-c3ccccc3-c3c(cccc3-c3ccccc3)O4)cc21 ; c1ccc(-c2ccc(N(c3ccc(-c4ccccc4)cc3)c3cccc4c3-c3ccccc3-c3c(cccc3-c3ccccc3)O4)cc2)cc1 ; CC1(C)c2ccccc2-c2ccc(N(c3ccc4c(c3)C(C)(C)c3ccccc3-4)c3cccc4c3-c3ccccc3-c3c(cccc3-c3ccccc3)O4)cc21]

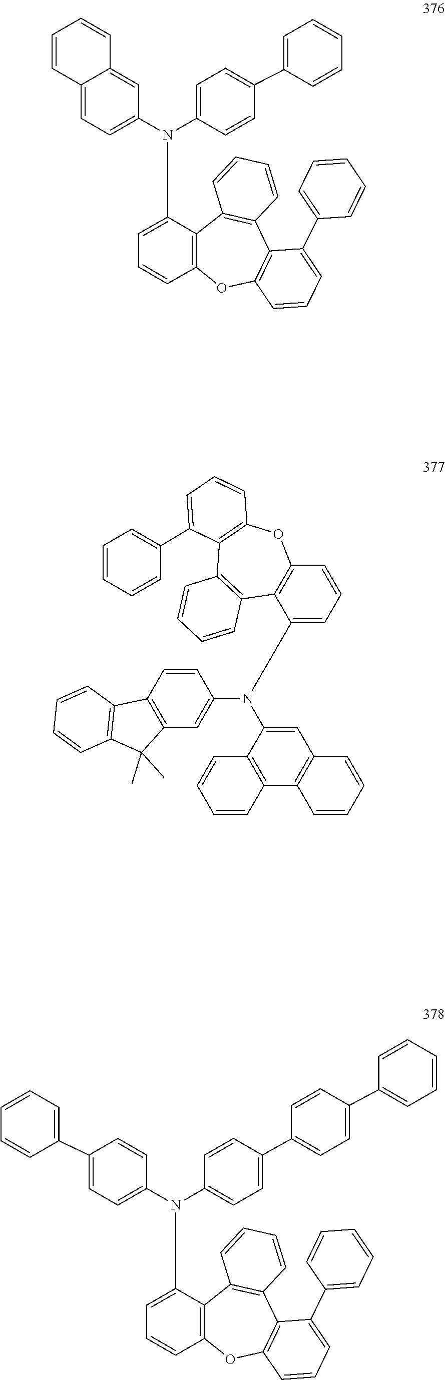 [SMILES: c1ccc(-c2ccc(N(c3ccc4ccccc4c3)c3cccc4c3-c3ccccc3-c3c(cccc3-c3ccccc3)O4)cc2)cc1 ; CC1(C)c2ccccc2-c2ccc(N(c3cccc4c3-c3ccccc3-c3c(cccc3-c3ccccc3)O4)c3cc4ccccc4c4ccccc34)cc21 ; c1ccc(-c2ccc(-c3ccc(N(c4ccc(-c5ccccc5)cc4)c4cccc5c4-c4ccccc4-c4c(cccc4-c4ccccc4)O5)cc3)cc2)cc1]

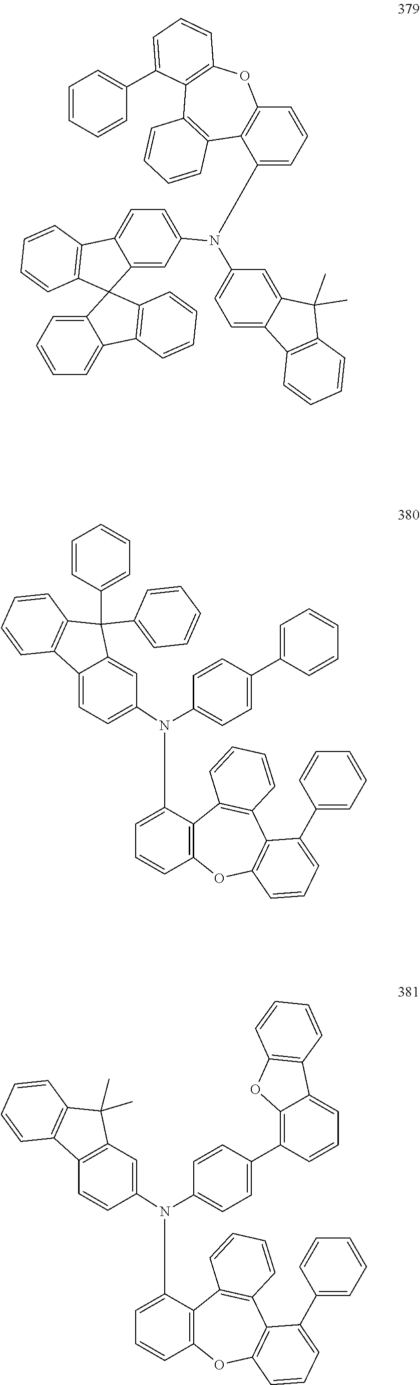 [SMILES: CC1(C)c2ccccc2-c2ccc(N(c3ccc4c(c3)C3(c5ccccc5-c5ccccc53)c3ccccc3-4)c3cccc4c3-c3ccccc3-c3c(cccc3-c3ccccc3)O4)cc21 ; c1ccc(-c2ccc(N(c3ccc4c(c3)C(c3ccccc3)(c3ccccc3)c3ccccc3-4)c3cccc4c3-c3ccccc3-c3c(cccc3-c3ccccc3)O4)cc2)cc1 ; CC1(C)c2ccccc2-c2ccc(N(c3ccc(-c4cccc5c4oc4ccccc45)cc3)c3cccc4c3-c3ccccc3-c3c(cccc3-c3ccccc3)O4)cc21]

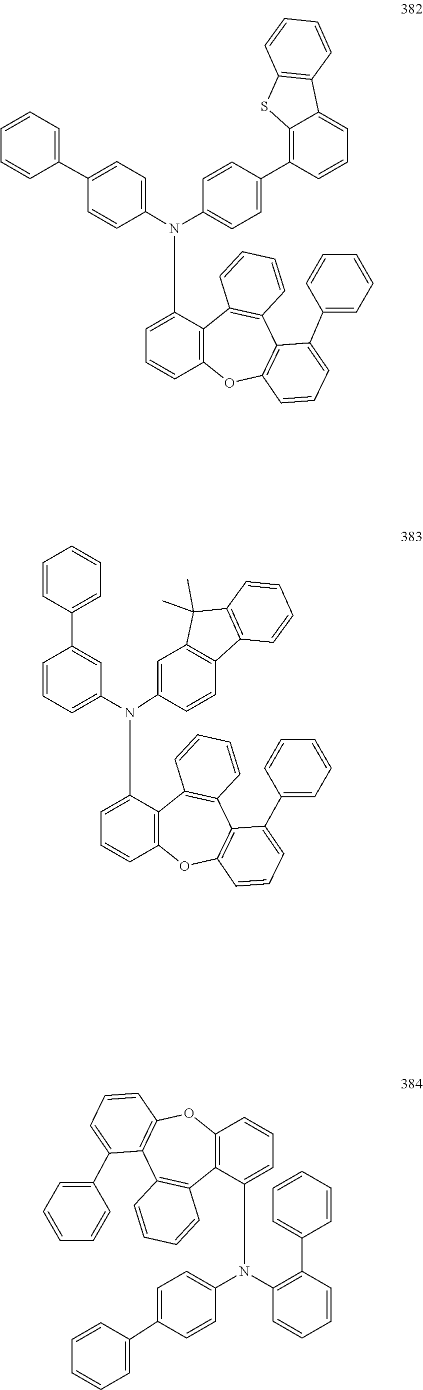 [SMILES: c1ccc(-c2ccc(N(c3ccc(-c4cccc5c4sc4ccccc45)cc3)c3cccc4c3-c3ccccc3-c3c(cccc3-c3ccccc3)O4)cc2)cc1 ; CC1(C)c2ccccc2-c2ccc(N(c3cccc(-c4ccccc4)c3)c3cccc4c3-c3ccccc3-c3c(cccc3-c3ccccc3)O4)cc21 ; c1ccc(-c2ccc(N(c3ccccc3-c3ccccc3)c3cccc4c3-c3ccccc3-c3c(cccc3-c3ccccc3)O4)cc2)cc1]

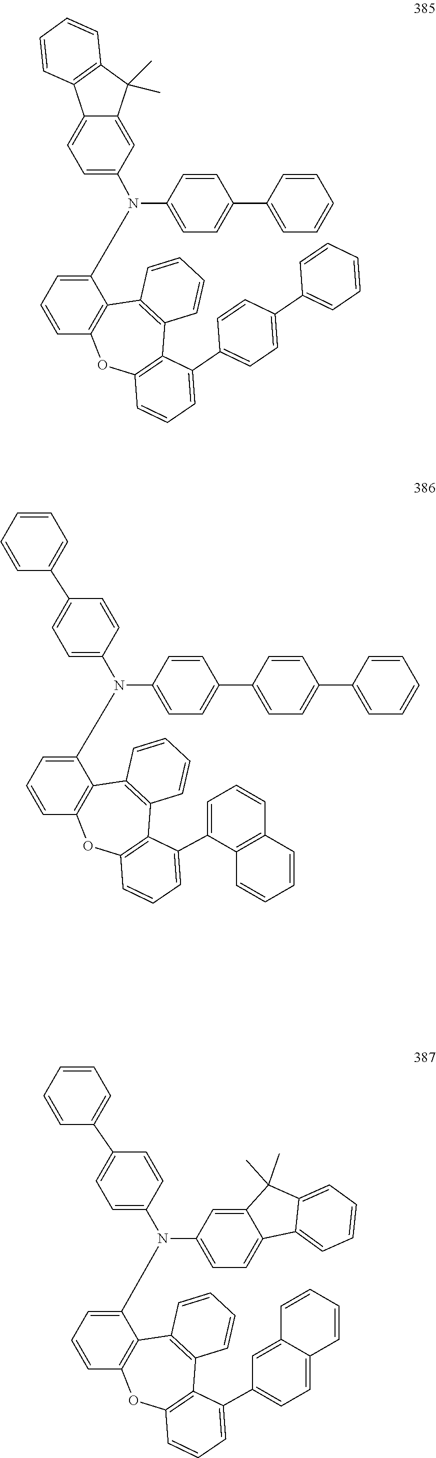 [SMILES: CC1(C)c2ccccc2-c2ccc(N(c3ccc(-c4ccccc4)cc3)c3cccc4c3-c3ccccc3-c3c(cccc3-c3ccc(-c5ccccc5)cc3)O4)cc21 ; c1ccc(-c2ccc(-c3ccc(N(c4ccc(-c5ccccc5)cc4)c4cccc5c4-c4ccccc4-c4c(cccc4-c4cccc6ccccc46)O5)cc3)cc2)cc1 ; CC1(C)c2ccccc2-c2ccc(N(c3ccc(-c4ccccc4)cc3)c3cccc4c3-c3ccccc3-c3c(cccc3-c3ccc5ccccc5c3)O4)cc21]

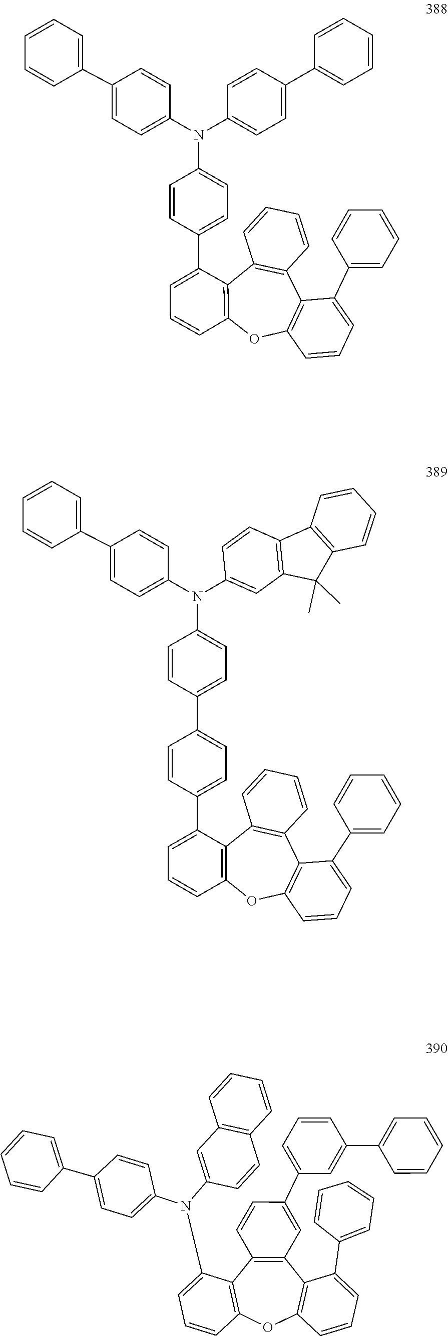 [SMILES: c1ccc(-c2ccc(N(c3ccc(-c4ccccc4)cc3)c3ccc(-c4cccc5c4-c4ccccc4-c4c(cccc4-c4ccccc4)O5)cc3)cc2)cc1 ; CC1(C)c2ccccc2-c2ccc(N(c3ccc(-c4ccccc4)cc3)c3ccc(-c4ccc(-c5cccc6c5-c5ccccc5-c5c(cccc5-c5ccccc5)O6)cc4)cc3)cc21 ; c1ccc(-c2ccc(N(c3ccc4ccccc4c3)c3cccc4c3-c3ccc(-c5cccc(-c6ccccc6)c5)cc3-c3c(cccc3-c3ccccc3)O4)cc2)cc1]

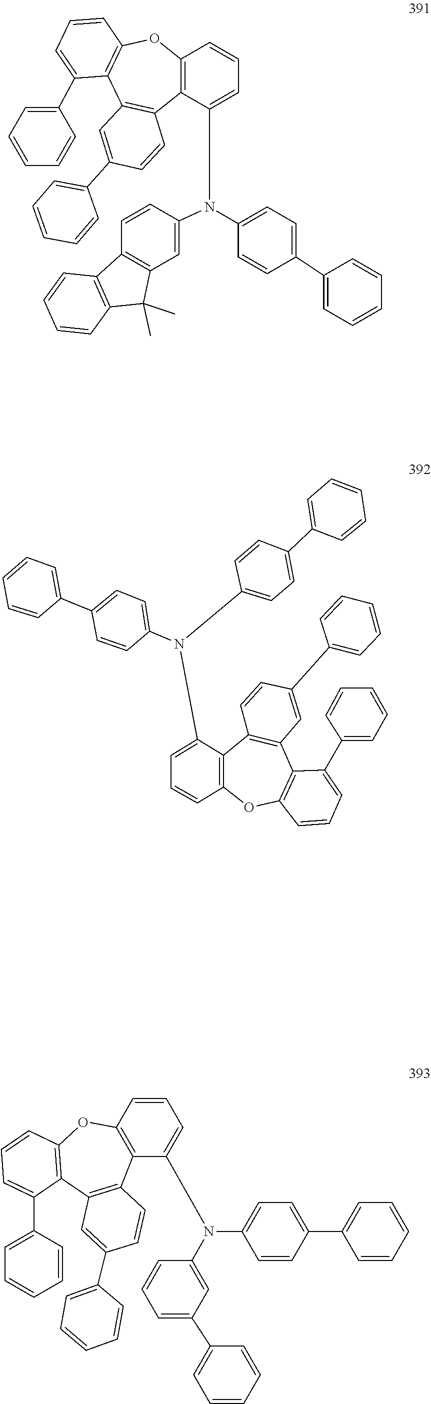 [SMILES: CC1(C)c2ccccc2-c2ccc(N(c3ccc(-c4ccccc4)cc3)c3cccc4c3-c3ccc(-c5ccccc5)cc3-c3c(cccc3-c3ccccc3)O4)cc21 ; c1ccc(-c2ccc(N(c3ccc(-c4ccccc4)cc3)c3cccc4c3-c3ccc(-c5ccccc5)cc3-c3c(cccc3-c3ccccc3)O4)cc2)cc1 ; c1ccc(-c2ccc(N(c3cccc(-c4ccccc4)c3)c3cccc4c3-c3ccc(-c5ccccc5)cc3-c3c(cccc3-c3ccccc3)O4)cc2)cc1]

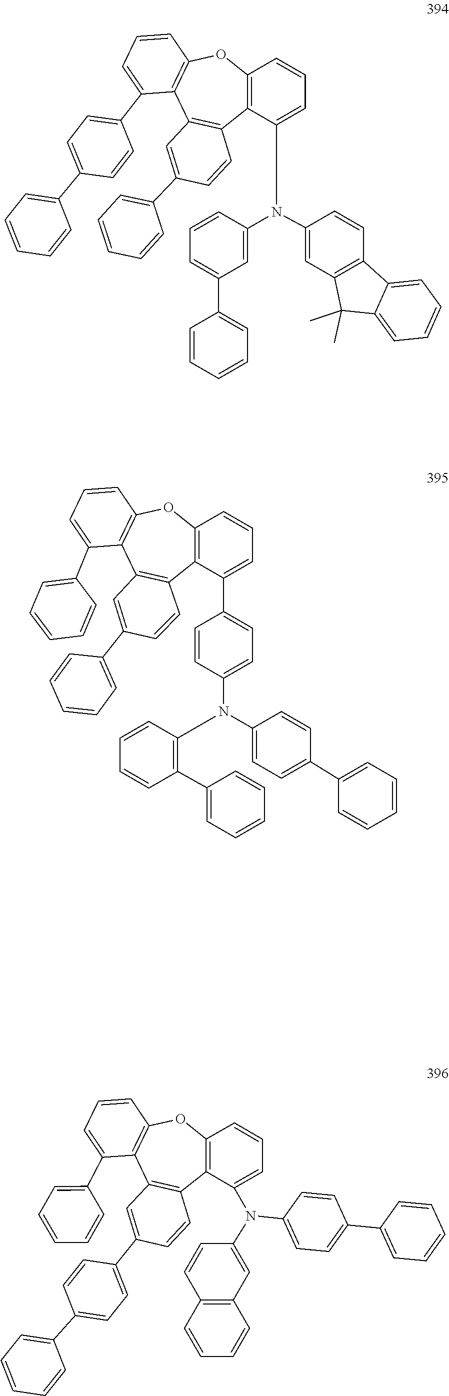 [SMILES: CC1(C)c2ccccc2-c2ccc(N(c3cccc(-c4ccccc4)c3)c3cccc4c3-c3ccc(-c5ccccc5)cc3-c3c(cccc3-c3ccc(-c5ccccc5)cc3)O4)cc21 ; c1ccc(-c2ccc(N(c3ccc(-c4cccc5c4-c4ccc(-c6ccccc6)cc4-c4c(cccc4-c4ccccc4)O5)cc3)c3ccccc3-c3ccccc3)cc2)cc1 ; c1ccc(-c2ccc(-c3ccc4c(c3)-c3c(cccc3-c3ccccc3)Oc3cccc(N(c5ccc(-c6ccccc6)cc5)c5ccc6ccccc6c5)c3-4)cc2)cc1]

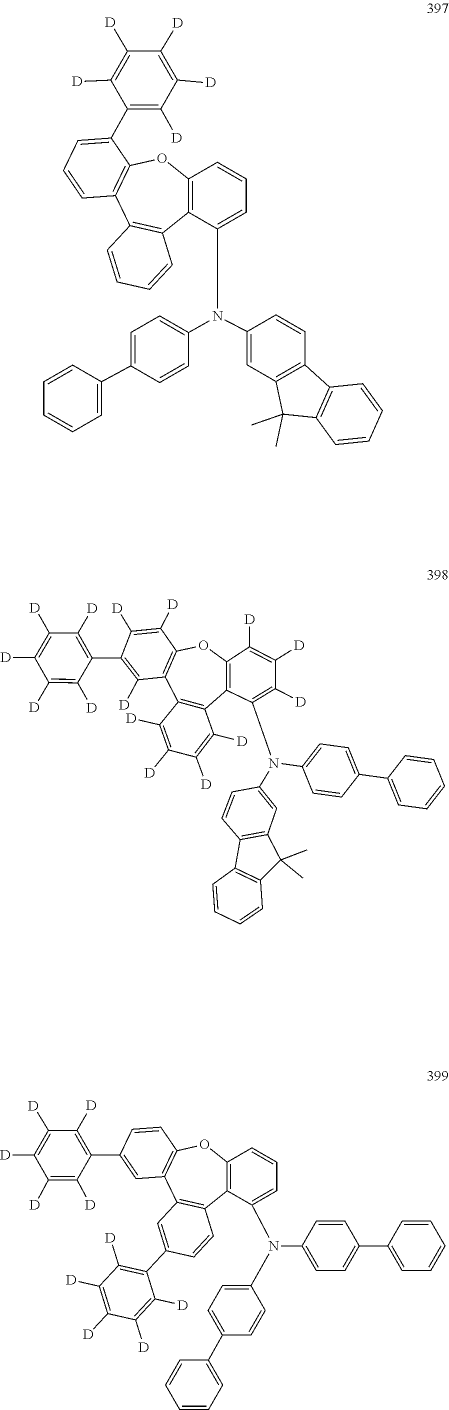 [SMILES: [2H]c1c([2H])c([2H])c(-c2cccc3c2Oc2cccc(N(c4ccc(-c5ccccc5)cc4)c4ccc5c(c4)C(C)(C)c4ccccc4-5)c2-c2ccccc2-3)c([2H])c1[2H] ; [2H]c1c([2H])c([2H])c(-c2c([2H])c([2H])c3c(c2[2H])-c2c([2H])c([2H])c([2H])c([2H])c2-c2c(c([2H])c([2H])c([2H])c2N(c2ccc(-c4ccccc4)cc2)c2ccc4c(c2)C(C)(C)c2ccccc2-4)O3)c([2H])c1[2H] ; [2H]c1c([2H])c([2H])c(-c2ccc3c(c2)-c2cc(-c4c([2H])c([2H])c([2H])c([2H])c4[2H])ccc2-c2c(cccc2N(c2ccc(-c4ccccc4)cc2)c2ccc(-c4ccccc4)cc2)O3)c([2H])c1[2H]]

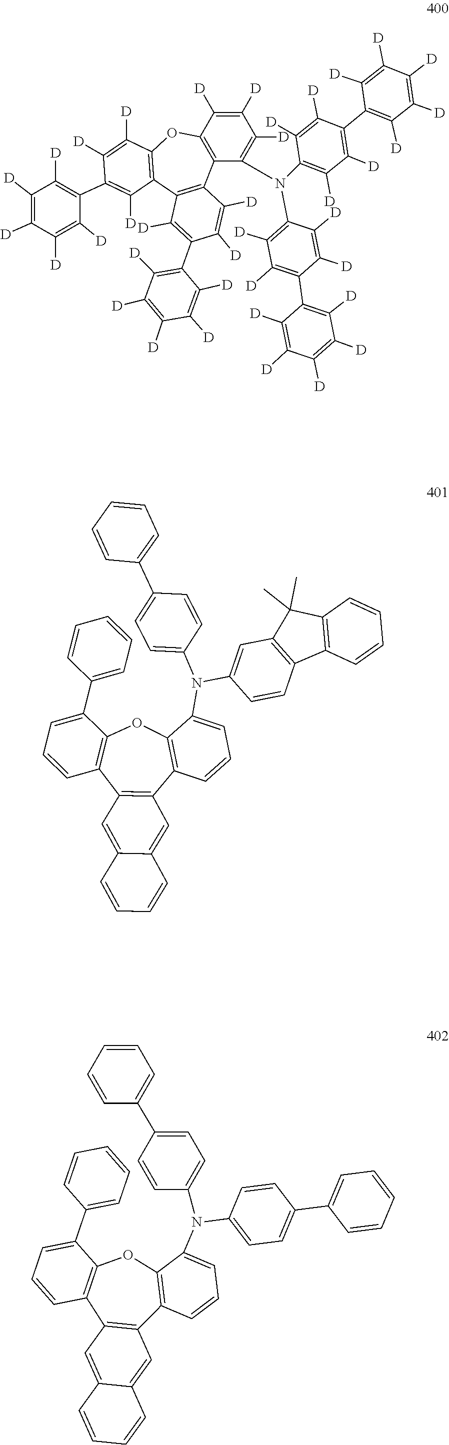 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c([2H])c4c3-c3c([2H])c([2H])c(-c5c([2H])c([2H])c([2H])c([2H])c5[2H])c([2H])c3-c3c([2H])c(-c5c([2H])c([2H])c([2H])c([2H])c5[2H])c([2H])c([2H])c3O4)c([2H])c2[2H])c([2H])c1[2H] ; CC1(C)c2ccccc2-c2ccc(N(c3ccc(-c4ccccc4)cc3)c3cccc4c3Oc3c(-c5ccccc5)cccc3-c3cc5ccccc5cc3-4)cc21 ; c1ccc(-c2ccc(N(c3ccc(-c4ccccc4)cc3)c3cccc4c3Oc3c(-c5ccccc5)cccc3-c3cc5ccccc5cc3-4)cc2)cc1]

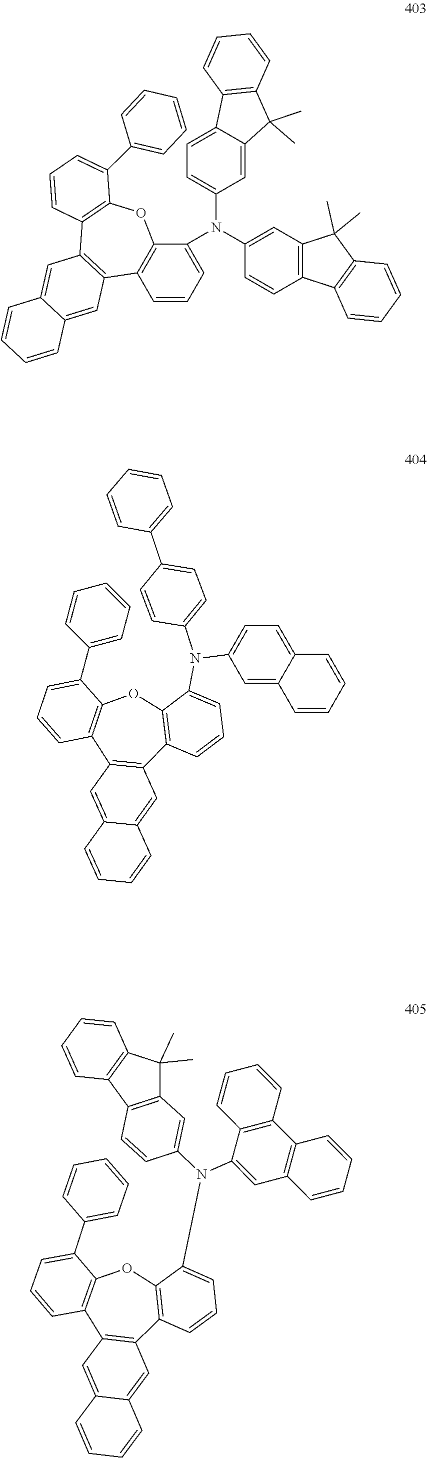 [SMILES: CC1(C)c2ccccc2-c2ccc(N(c3ccc4c(c3)C(C)(C)c3ccccc3-4)c3cccc4c3Oc3c(-c5ccccc5)cccc3-c3cc5ccccc5cc3-4)cc21 ; c1ccc(-c2ccc(N(c3ccc4ccccc4c3)c3cccc4c3Oc3c(-c5ccccc5)cccc3-c3cc5ccccc5cc3-4)cc2)cc1 ; CC1(C)c2ccccc2-c2ccc(N(c3cccc4c3Oc3c(-c5ccccc5)cccc3-c3cc5ccccc5cc3-4)c3cc4ccccc4c4ccccc34)cc21]

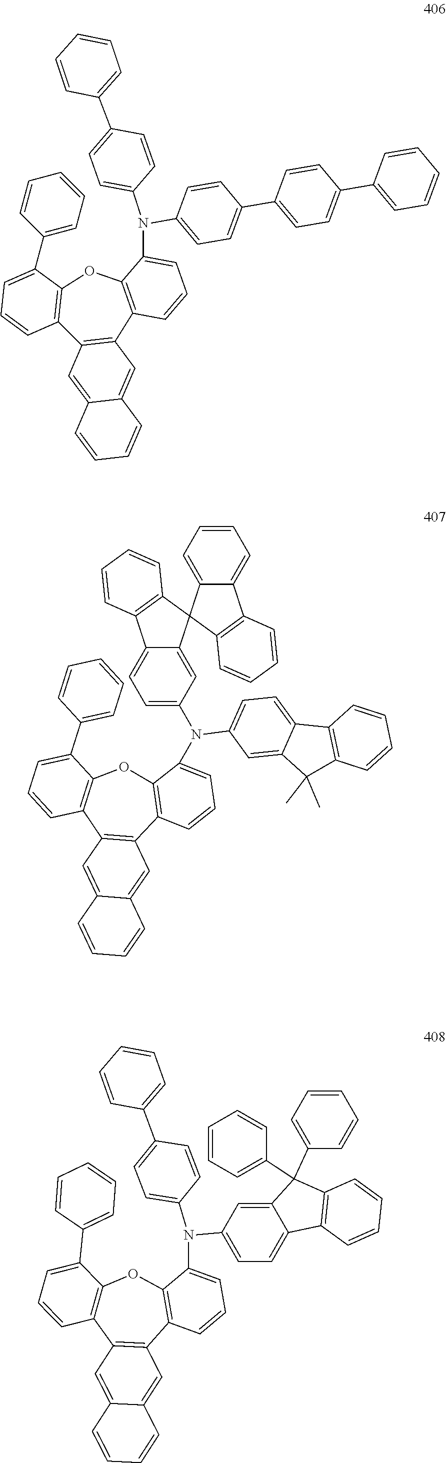 [SMILES: c1ccc(-c2ccc(-c3ccc(N(c4ccc(-c5ccccc5)cc4)c4cccc5c4Oc4c(-c6ccccc6)cccc4-c4cc6ccccc6cc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3c(-c5ccccc5)cccc3-c3cc5ccccc5cc3-4)cc21 ; c1ccc(-c2ccc(N(c3ccc4c(c3)C(c3ccccc3)(c3ccccc3)c3ccccc3-4)c3cccc4c3Oc3c(-c5ccccc5)cccc3-c3cc5ccccc5cc3-4)cc2)cc1]

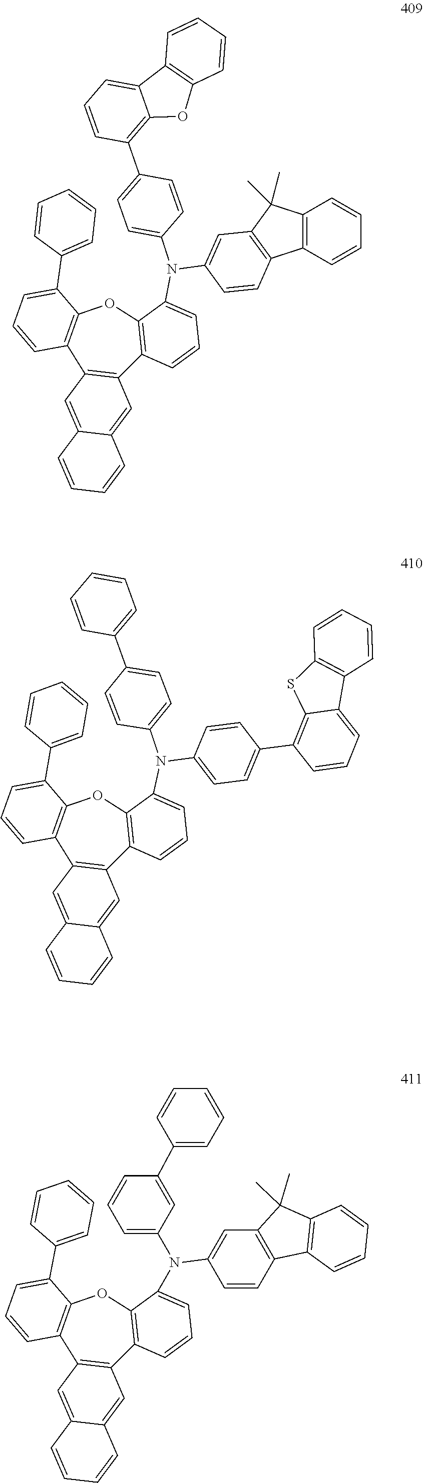 [SMILES: CC1(C)c2ccccc2-c2ccc(N(c3ccc(-c4cccc5c4oc4ccccc45)cc3)c3cccc4c3Oc3c(-c5ccccc5)cccc3-c3cc5ccccc5cc3-4)cc21 ; c1ccc(-c2ccc(N(c3ccc(-c4cccc5c4sc4ccccc45)cc3)c3cccc4c3Oc3c(-c5ccccc5)cccc3-c3cc5ccccc5cc3-4)cc2)cc1 ; CC1(C)c2ccccc2-c2ccc(N(c3cccc(-c4ccccc4)c3)c3cccc4c3Oc3c(-c5ccccc5)cccc3-c3cc5ccccc5cc3-4)cc21]

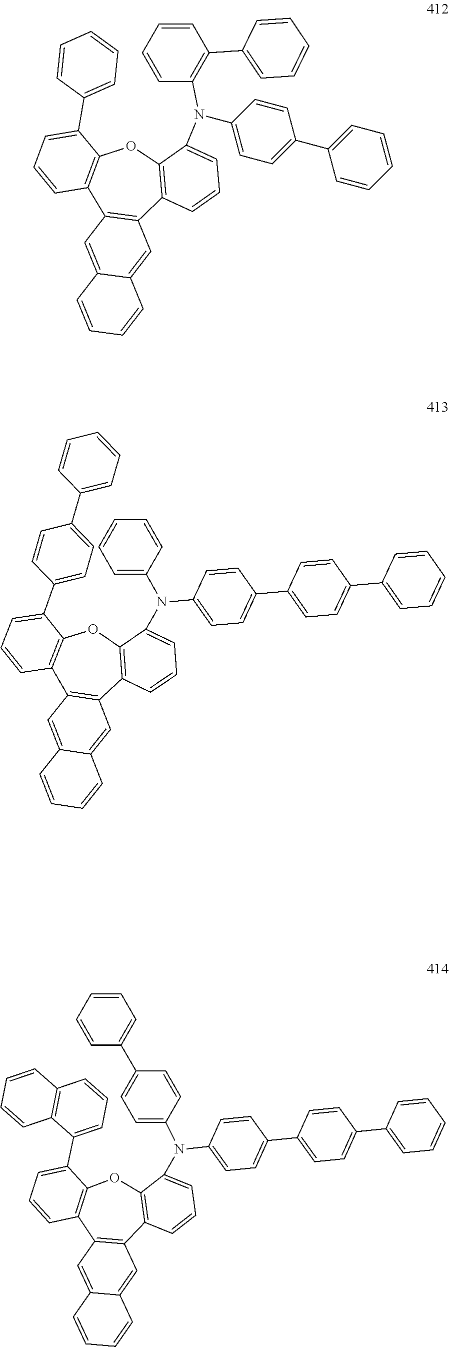 [SMILES: c1ccc(-c2ccc(N(c3ccccc3-c3ccccc3)c3cccc4c3Oc3c(-c5ccccc5)cccc3-c3cc5ccccc5cc3-4)cc2)cc1 ; c1ccc(-c2ccc(-c3ccc(N(c4ccccc4)c4cccc5c4Oc4c(-c6ccc(-c7ccccc7)cc6)cccc4-c4cc6ccccc6cc4-5)cc3)cc2)cc1 ; c1ccc(-c2ccc(-c3ccc(N(c4ccc(-c5ccccc5)cc4)c4cccc5c4Oc4c(cccc4-c4cccc6ccccc46)-c4cc6ccccc6cc4-5)cc3)cc2)cc1]

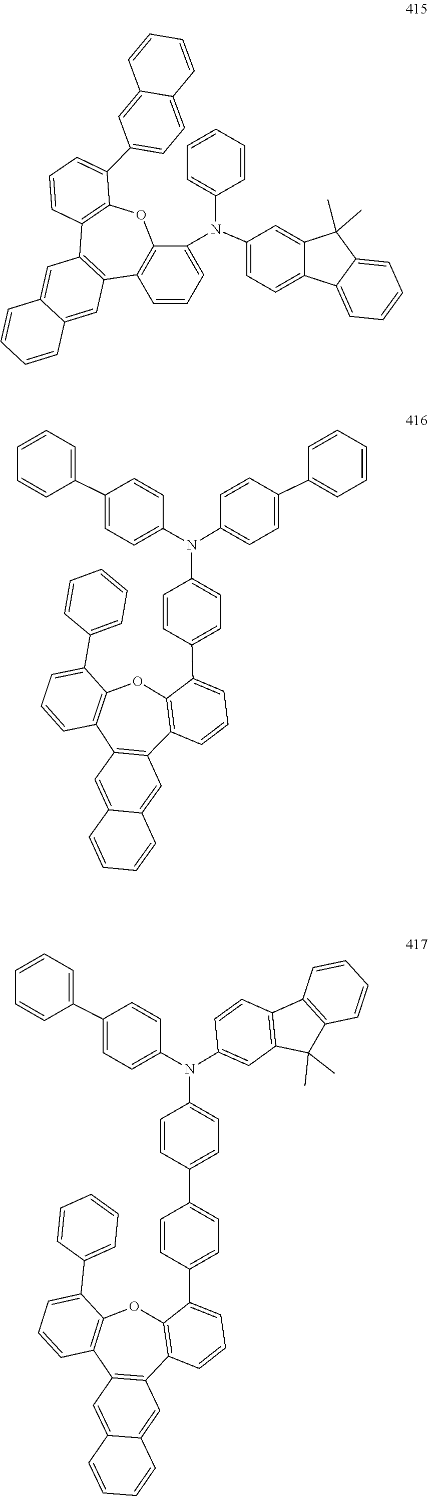 [SMILES: CC1(C)c2ccccc2-c2ccc(N(c3ccccc3)c3cccc4c3Oc3c(-c5ccc6ccccc6c5)cccc3-c3cc5ccccc5cc3-4)cc21 ; c1ccc(-c2ccc(N(c3ccc(-c4ccccc4)cc3)c3ccc(-c4cccc5c4Oc4c(-c6ccccc6)cccc4-c4cc6ccccc6cc4-5)cc3)cc2)cc1 ; CC1(C)c2ccccc2-c2ccc(N(c3ccc(-c4ccccc4)cc3)c3ccc(-c4ccc(-c5cccc6c5Oc5c(-c7ccccc7)cccc5-c5cc7ccccc7cc5-6)cc4)cc3)cc21]

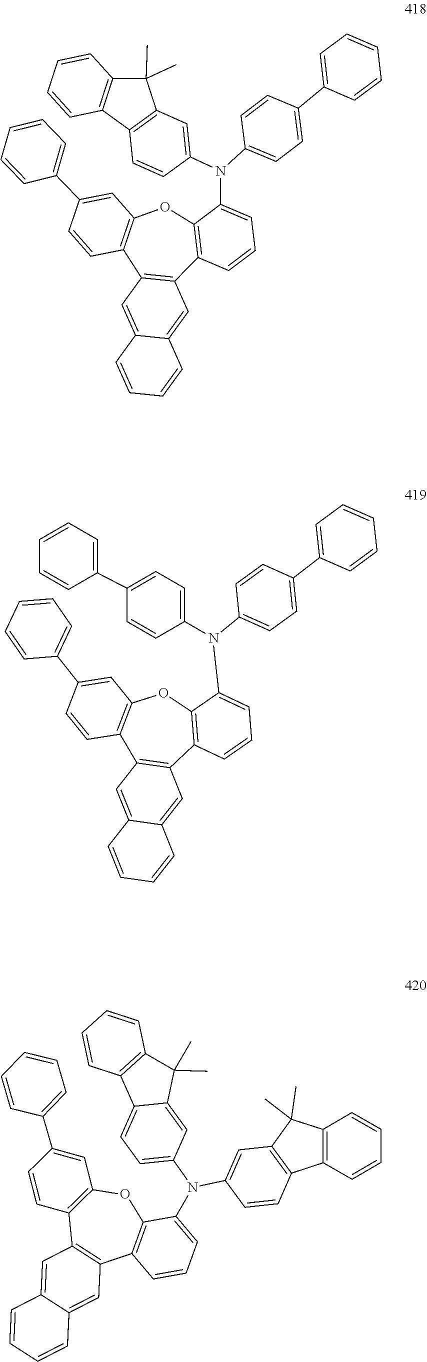 [SMILES: CC1(C)c2ccccc2-c2ccc(N(c3ccc(-c4ccccc4)cc3)c3cccc4c3Oc3cc(-c5ccccc5)ccc3-c3cc5ccccc5cc3-4)cc21 ; c1ccc(-c2ccc(N(c3ccc(-c4ccccc4)cc3)c3cccc4c3Oc3cc(-c5ccccc5)ccc3-c3cc5ccccc5cc3-4)cc2)cc1 ; CC1(C)c2ccccc2-c2ccc(N(c3ccc4c(c3)C(C)(C)c3ccccc3-4)c3cccc4c3Oc3cc(-c5ccccc5)ccc3-c3cc5ccccc5cc3-4)cc21]

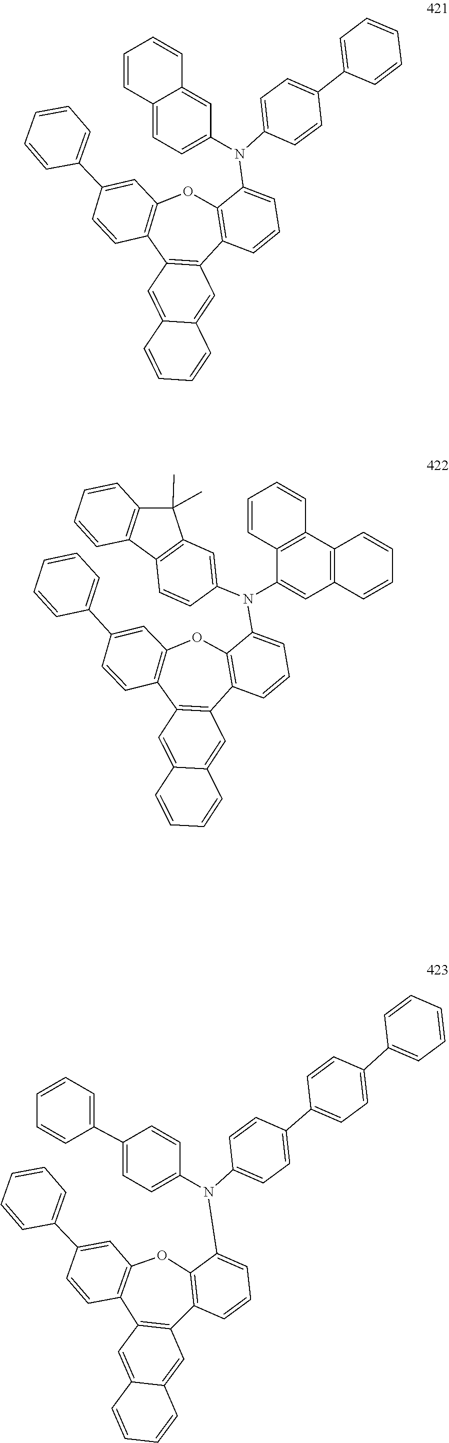 [SMILES: c1ccc(-c2ccc(N(c3ccc4ccccc4c3)c3cccc4c3Oc3cc(-c5ccccc5)ccc3-c3cc5ccccc5cc3-4)cc2)cc1 ; CC1(C)c2ccccc2-c2ccc(N(c3cccc4c3Oc3cc(-c5ccccc5)ccc3-c3cc5ccccc5cc3-4)c3cc4ccccc4c4ccccc34)cc21 ; c1ccc(-c2ccc(-c3ccc(N(c4ccc(-c5ccccc5)cc4)c4cccc5c4Oc4cc(-c6ccccc6)ccc4-c4cc6ccccc6cc4-5)cc3)cc2)cc1]

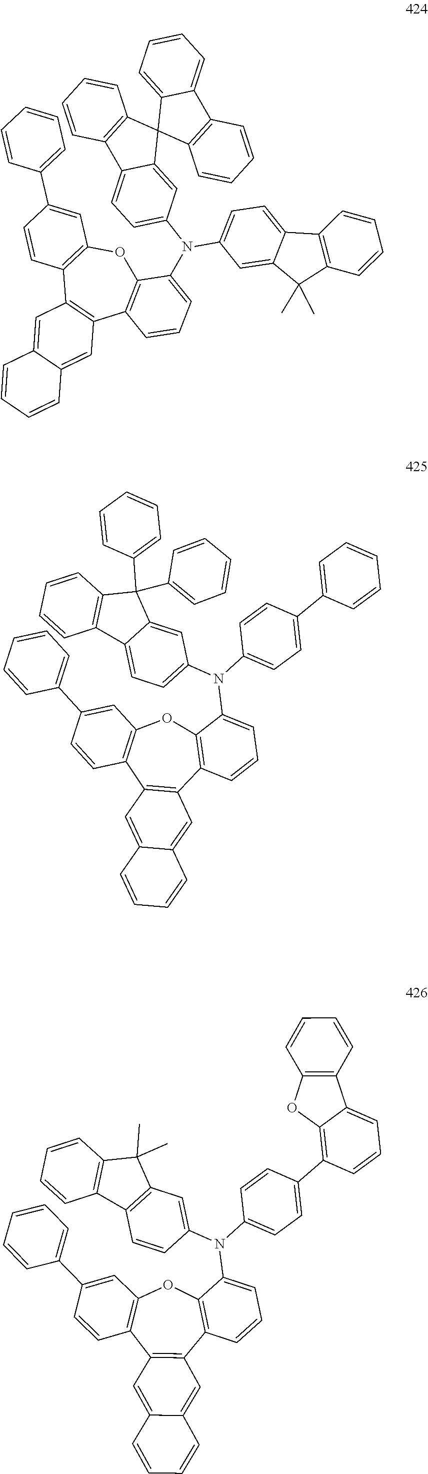 [SMILES: CC1(C)c2ccccc2-c2ccc(N(c3ccc4c(c3)C3(c5ccccc5-c5ccccc53)c3ccccc3-4)c3cccc4c3Oc3cc(-c5ccccc5)ccc3-c3cc5ccccc5cc3-4)cc21 ; c1ccc(-c2ccc(N(c3ccc4c(c3)C(c3ccccc3)(c3ccccc3)c3ccccc3-4)c3cccc4c3Oc3cc(-c5ccccc5)ccc3-c3cc5ccccc5cc3-4)cc2)cc1 ; CC1(C)c2ccccc2-c2ccc(N(c3ccc(-c4cccc5c4oc4ccccc45)cc3)c3cccc4c3Oc3cc(-c5ccccc5)ccc3-c3cc5ccccc5cc3-4)cc21]

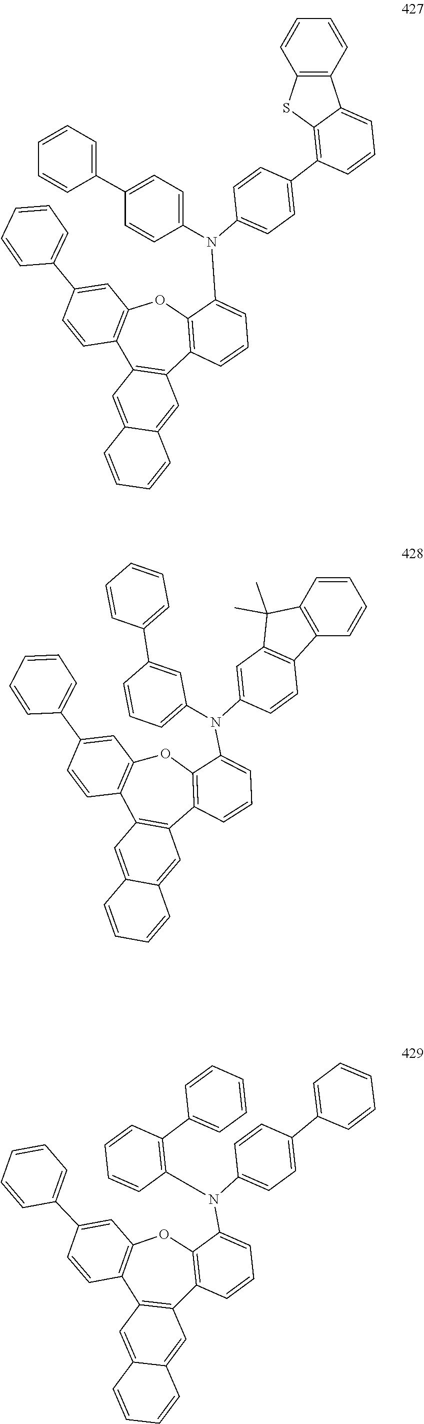 [SMILES: c1ccc(-c2ccc(N(c3ccc(-c4cccc5c4sc4ccccc45)cc3)c3cccc4c3Oc3cc(-c5ccccc5)ccc3-c3cc5ccccc5cc3-4)cc2)cc1 ; CC1(C)c2ccccc2-c2ccc(N(c3cccc(-c4ccccc4)c3)c3cccc4c3Oc3cc(-c5ccccc5)ccc3-c3cc5ccccc5cc3-4)cc21 ; c1ccc(-c2ccc(N(c3ccccc3-c3ccccc3)c3cccc4c3Oc3cc(-c5ccccc5)ccc3-c3cc5ccccc5cc3-4)cc2)cc1]

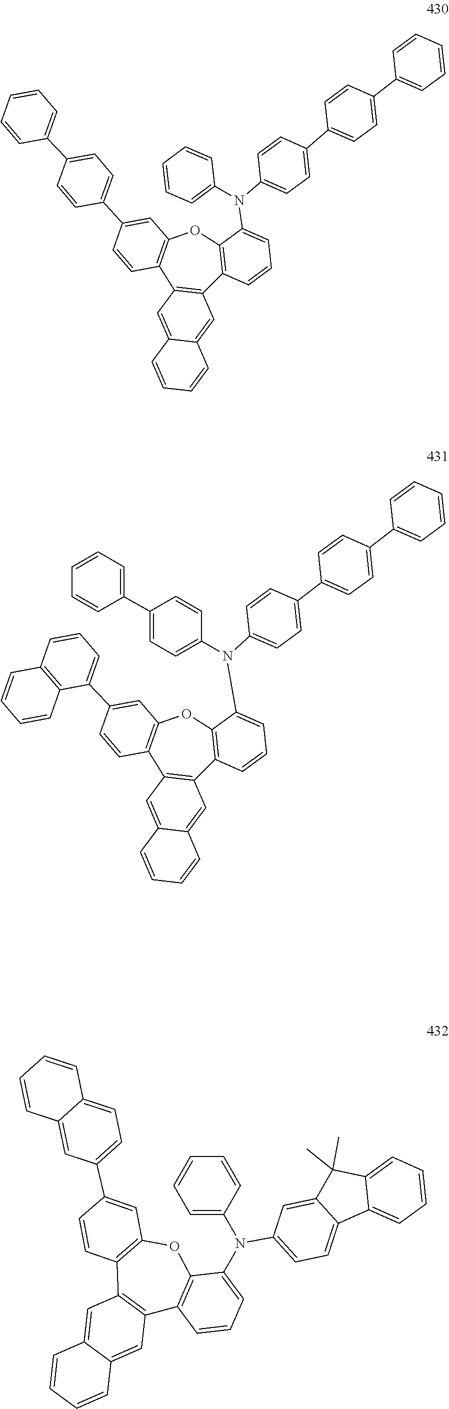 [SMILES: c1ccc(-c2ccc(-c3ccc(N(c4ccccc4)c4cccc5c4Oc4cc(-c6ccc(-c7ccccc7)cc6)ccc4-c4cc6ccccc6cc4-5)cc3)cc2)cc1 ; c1ccc(-c2ccc(-c3ccc(N(c4ccc(-c5ccccc5)cc4)c4cccc5c4Oc4cc(-c6cccc7ccccc67)ccc4-c4cc6ccccc6cc4-5)cc3)cc2)cc1 ; CC1(C)c2ccccc2-c2ccc(N(c3ccccc3)c3cccc4c3Oc3cc(-c5ccc6ccccc6c5)ccc3-c3cc5ccccc5cc3-4)cc21]

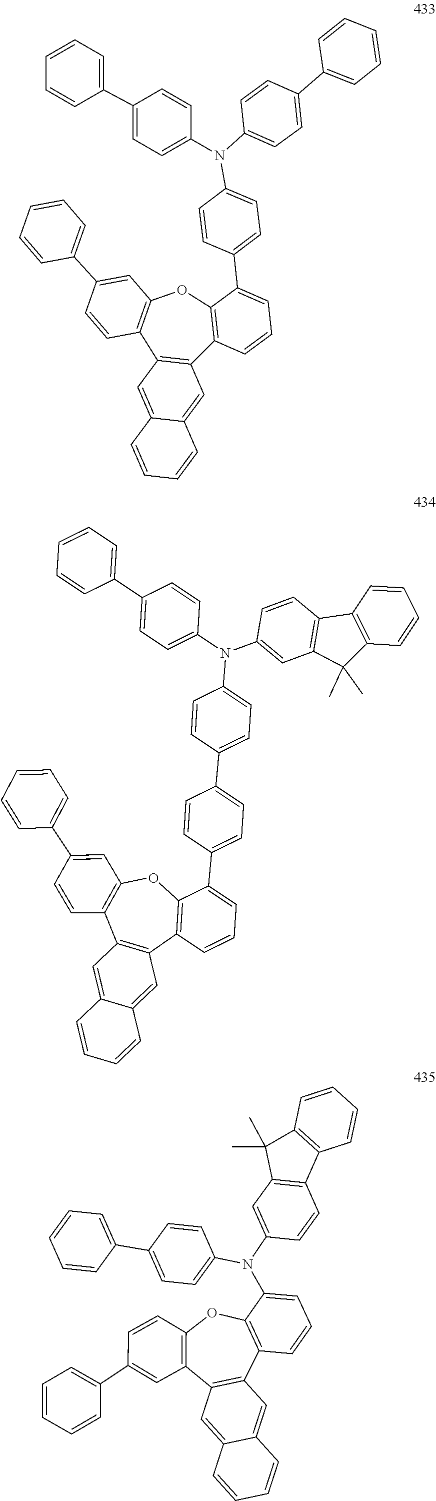 [SMILES: c1ccc(-c2ccc(N(c3ccc(-c4ccccc4)cc3)c3ccc(-c4cccc5c4Oc4cc(-c6ccccc6)ccc4-c4cc6ccccc6cc4-5)cc3)cc2)cc1 ; CC1(C)c2ccccc2-c2ccc(N(c3ccc(-c4ccccc4)cc3)c3ccc(-c4ccc(-c5cccc6c5Oc5cc(-c7ccccc7)ccc5-c5cc7ccccc7cc5-6)cc4)cc3)cc21 ; CC1(C)c2ccccc2-c2ccc(N(c3ccc(-c4ccccc4)cc3)c3cccc4c3Oc3ccc(-c5ccccc5)cc3-c3cc5ccccc5cc3-4)cc21]

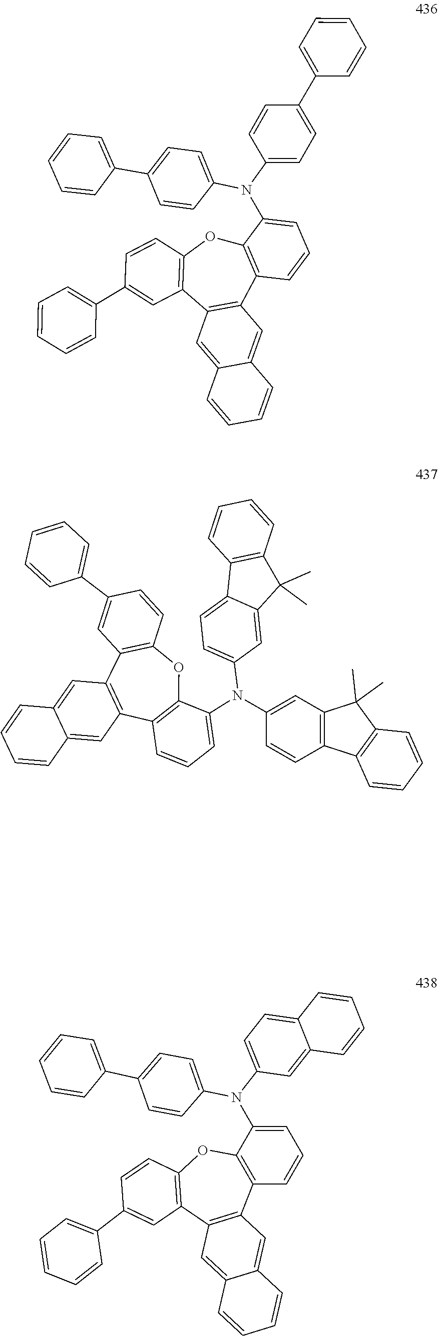 [SMILES: c1ccc(-c2ccc(N(c3ccc(-c4ccccc4)cc3)c3cccc4c3Oc3ccc(-c5ccccc5)cc3-c3cc5ccccc5cc3-4)cc2)cc1 ; CC1(C)c2ccccc2-c2ccc(N(c3ccc4c(c3)C(C)(C)c3ccccc3-4)c3cccc4c3Oc3ccc(-c5ccccc5)cc3-c3cc5ccccc5cc3-4)cc21 ; c1ccc(-c2ccc(N(c3ccc4ccccc4c3)c3cccc4c3Oc3ccc(-c5ccccc5)cc3-c3cc5ccccc5cc3-4)cc2)cc1]

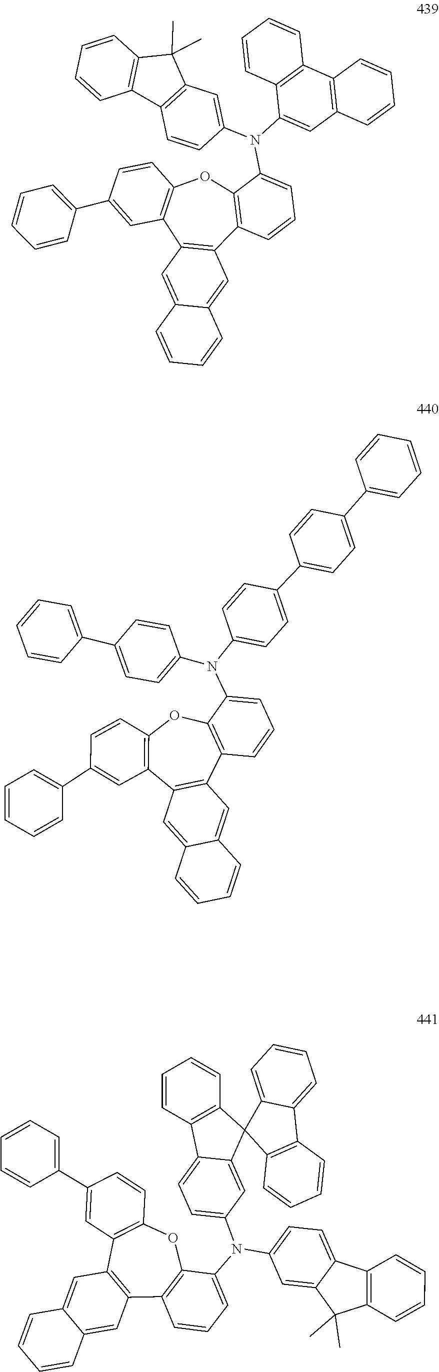 [SMILES: CC1(C)c2ccccc2-c2ccc(N(c3cccc4c3Oc3ccc(-c5ccccc5)cc3-c3cc5ccccc5cc3-4)c3cc4ccccc4c4ccccc34)cc21 ; c1ccc(-c2ccc(-c3ccc(N(c4ccc(-c5ccccc5)cc4)c4cccc5c4Oc4ccc(-c6ccccc6)cc4-c4cc6ccccc6cc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3ccc(-c5ccccc5)cc3-c3cc5ccccc5cc3-4)cc21]

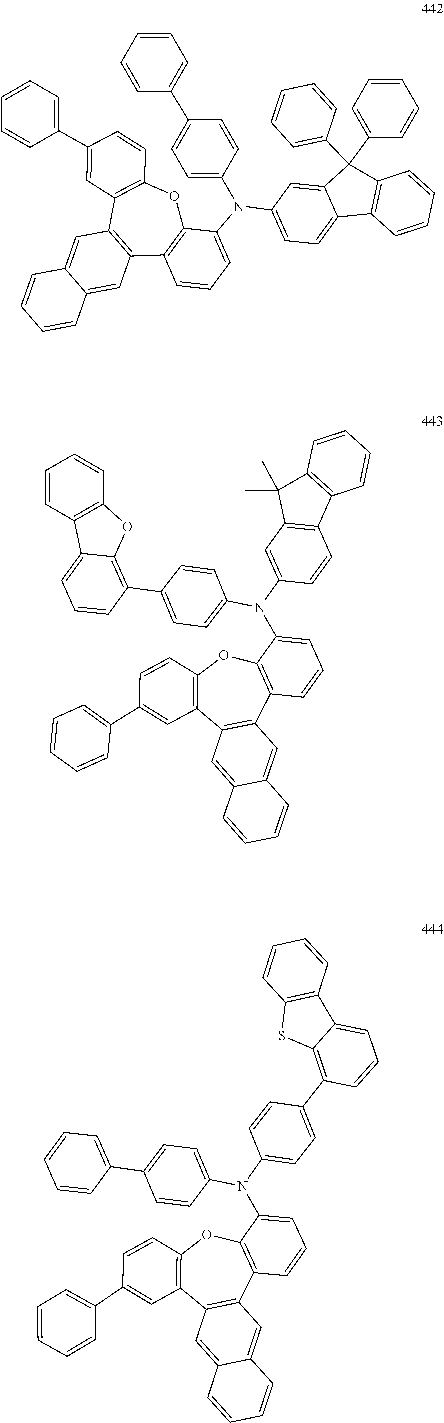 [SMILES: c1ccc(-c2ccc(N(c3ccc4c(c3)C(c3ccccc3)(c3ccccc3)c3ccccc3-4)c3cccc4c3Oc3ccc(-c5ccccc5)cc3-c3cc5ccccc5cc3-4)cc2)cc1 ; CC1(C)c2ccccc2-c2ccc(N(c3ccc(-c4cccc5c4oc4ccccc45)cc3)c3cccc4c3Oc3ccc(-c5ccccc5)cc3-c3cc5ccccc5cc3-4)cc21 ; c1ccc(-c2ccc(N(c3ccc(-c4cccc5c4sc4ccccc45)cc3)c3cccc4c3Oc3ccc(-c5ccccc5)cc3-c3cc5ccccc5cc3-4)cc2)cc1]

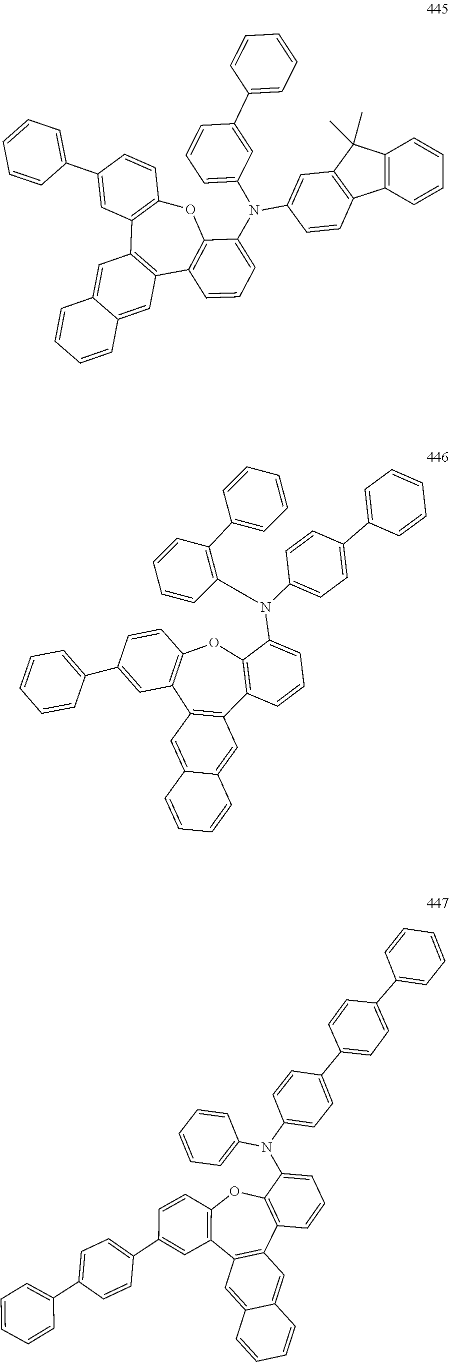 [SMILES: CC1(C)c2ccccc2-c2ccc(N(c3cccc(-c4ccccc4)c3)c3cccc4c3Oc3ccc(-c5ccccc5)cc3-c3cc5ccccc5cc3-4)cc21 ; c1ccc(-c2ccc(N(c3ccccc3-c3ccccc3)c3cccc4c3Oc3ccc(-c5ccccc5)cc3-c3cc5ccccc5cc3-4)cc2)cc1 ; c1ccc(-c2ccc(-c3ccc(N(c4ccccc4)c4cccc5c4Oc4ccc(-c6ccc(-c7ccccc7)cc6)cc4-c4cc6ccccc6cc4-5)cc3)cc2)cc1]

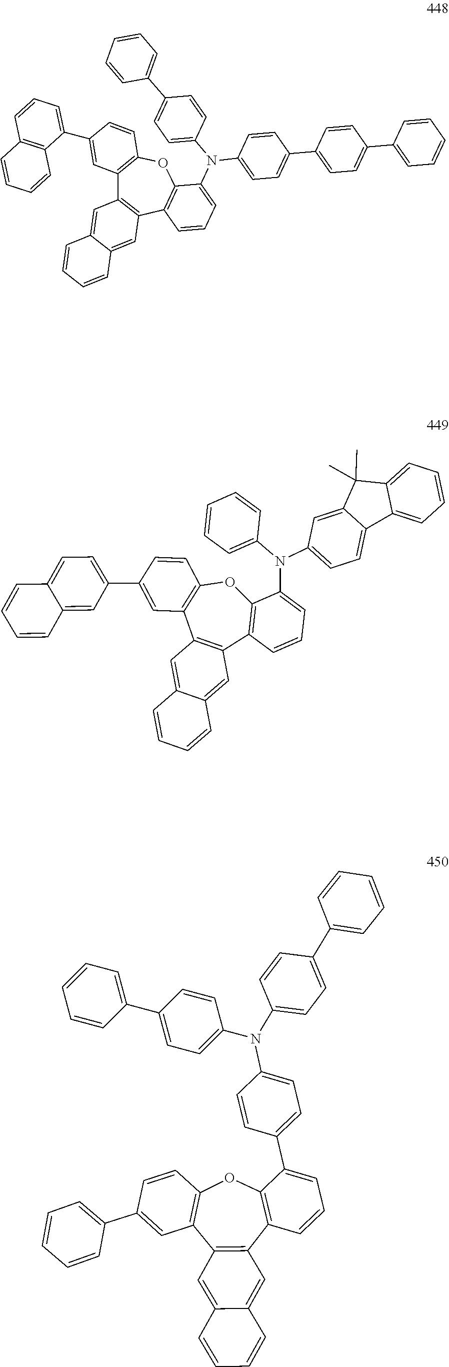 [SMILES: c1ccc(-c2ccc(-c3ccc(N(c4ccc(-c5ccccc5)cc4)c4cccc5c4Oc4ccc(-c6cccc7ccccc67)cc4-c4cc6ccccc6cc4-5)cc3)cc2)cc1 ; CC1(C)c2ccccc2-c2ccc(N(c3ccccc3)c3cccc4c3Oc3ccc(-c5ccc6ccccc6c5)cc3-c3cc5ccccc5cc3-4)cc21 ; c1ccc(-c2ccc(N(c3ccc(-c4ccccc4)cc3)c3ccc(-c4cccc5c4Oc4ccc(-c6ccccc6)cc4-c4cc6ccccc6cc4-5)cc3)cc2)cc1]

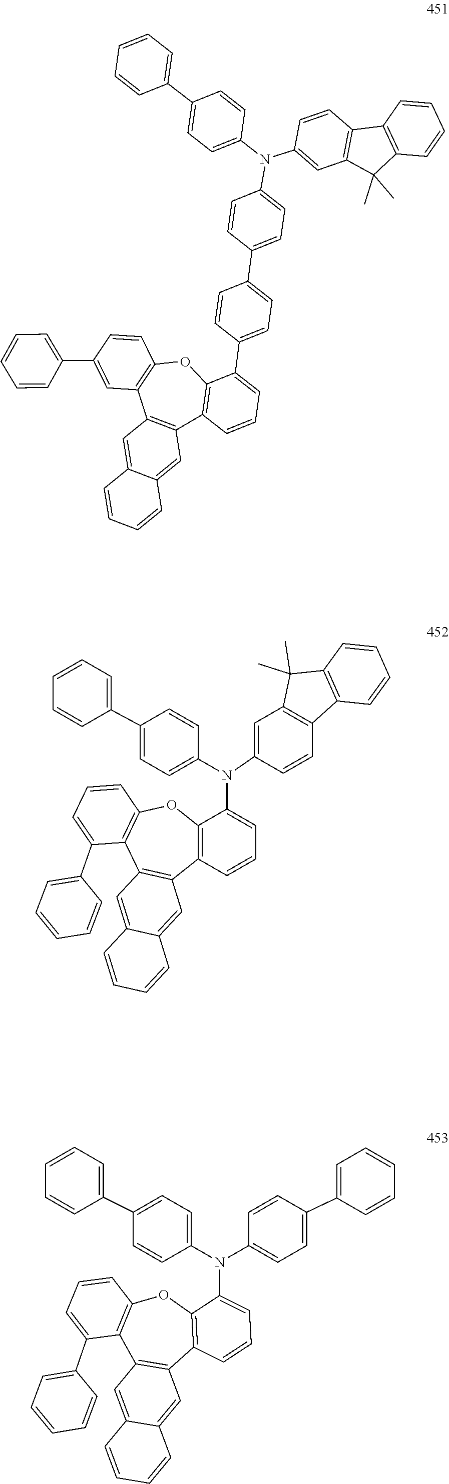 [SMILES: CC1(C)c2ccccc2-c2ccc(N(c3ccc(-c4ccccc4)cc3)c3ccc(-c4ccc(-c5cccc6c5Oc5ccc(-c7ccccc7)cc5-c5cc7ccccc7cc5-6)cc4)cc3)cc21 ; CC1(C)c2ccccc2-c2ccc(N(c3ccc(-c4ccccc4)cc3)c3cccc4c3Oc3cccc(-c5ccccc5)c3-c3cc5ccccc5cc3-4)cc21 ; c1ccc(-c2ccc(N(c3ccc(-c4ccccc4)cc3)c3cccc4c3Oc3cccc(-c5ccccc5)c3-c3cc5ccccc5cc3-4)cc2)cc1]

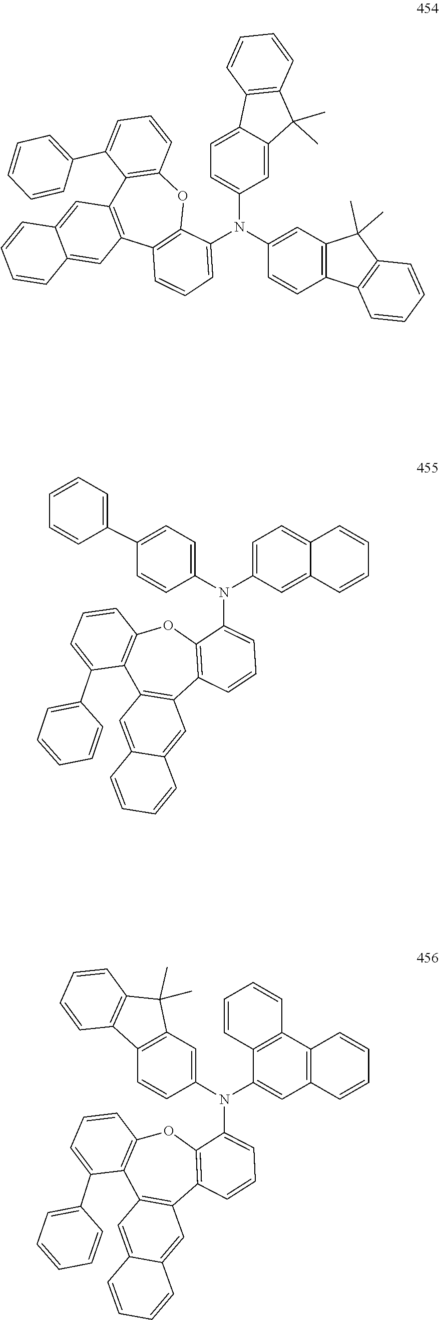 [SMILES: CC1(C)c2ccccc2-c2ccc(N(c3ccc4c(c3)C(C)(C)c3ccccc3-4)c3cccc4c3Oc3cccc(-c5ccccc5)c3-c3cc5ccccc5cc3-4)cc21 ; c1ccc(-c2ccc(N(c3ccc4ccccc4c3)c3cccc4c3Oc3cccc(-c5ccccc5)c3-c3cc5ccccc5cc3-4)cc2)cc1 ; CC1(C)c2ccccc2-c2ccc(N(c3cccc4c3Oc3cccc(-c5ccccc5)c3-c3cc5ccccc5cc3-4)c3cc4ccccc4c4ccccc34)cc21]

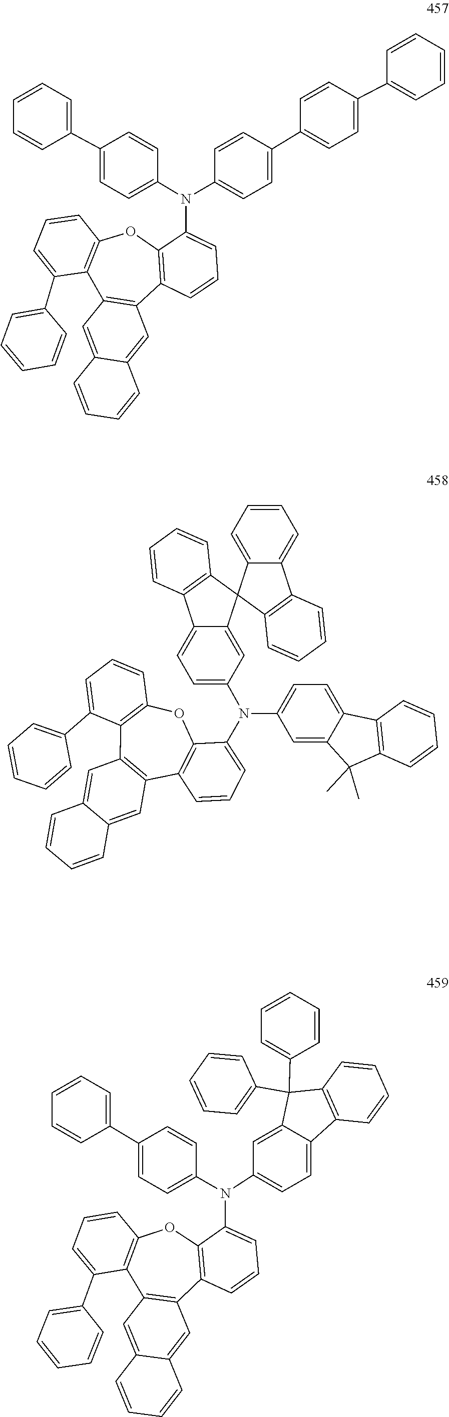 [SMILES: c1ccc(-c2ccc(-c3ccc(N(c4ccc(-c5ccccc5)cc4)c4cccc5c4Oc4cccc(-c6ccccc6)c4-c4cc6ccccc6cc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cccc(-c5ccccc5)c3-c3cc5ccccc5cc3-4)cc21 ; c1ccc(-c2ccc(N(c3ccc4c(c3)C(c3ccccc3)(c3ccccc3)c3ccccc3-4)c3cccc4c3Oc3cccc(-c5ccccc5)c3-c3cc5ccccc5cc3-4)cc2)cc1]

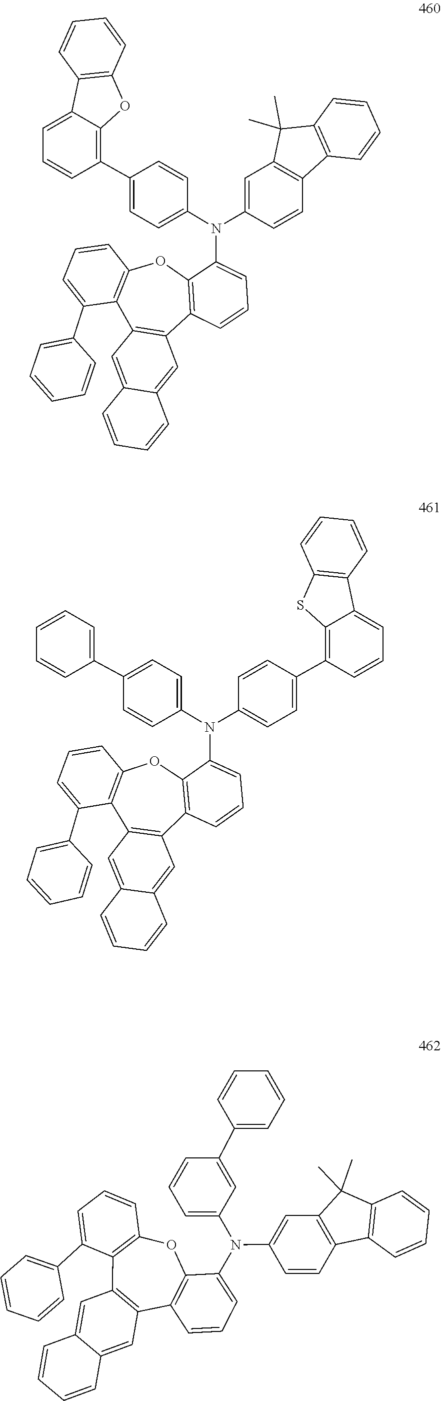 [SMILES: CC1(C)c2ccccc2-c2ccc(N(c3ccc(-c4cccc5c4oc4ccccc45)cc3)c3cccc4c3Oc3cccc(-c5ccccc5)c3-c3cc5ccccc5cc3-4)cc21 ; c1ccc(-c2ccc(N(c3ccc(-c4cccc5c4sc4ccccc45)cc3)c3cccc4c3Oc3cccc(-c5ccccc5)c3-c3cc5ccccc5cc3-4)cc2)cc1 ; CC1(C)c2ccccc2-c2ccc(N(c3cccc(-c4ccccc4)c3)c3cccc4c3Oc3cccc(-c5ccccc5)c3-c3cc5ccccc5cc3-4)cc21]

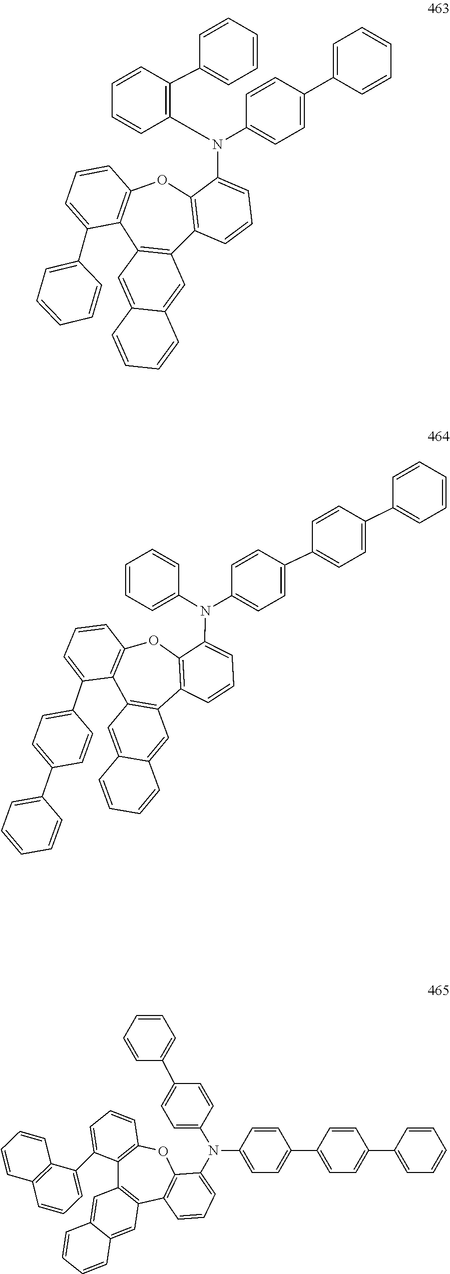 [SMILES: c1ccc(-c2ccc(N(c3ccccc3-c3ccccc3)c3cccc4c3Oc3cccc(-c5ccccc5)c3-c3cc5ccccc5cc3-4)cc2)cc1 ; c1ccc(-c2ccc(-c3ccc(N(c4ccccc4)c4cccc5c4Oc4cccc(-c6ccc(-c7ccccc7)cc6)c4-c4cc6ccccc6cc4-5)cc3)cc2)cc1 ; c1ccc(-c2ccc(-c3ccc(N(c4ccc(-c5ccccc5)cc4)c4cccc5c4Oc4cccc(-c6cccc7ccccc67)c4-c4cc6ccccc6cc4-5)cc3)cc2)cc1]

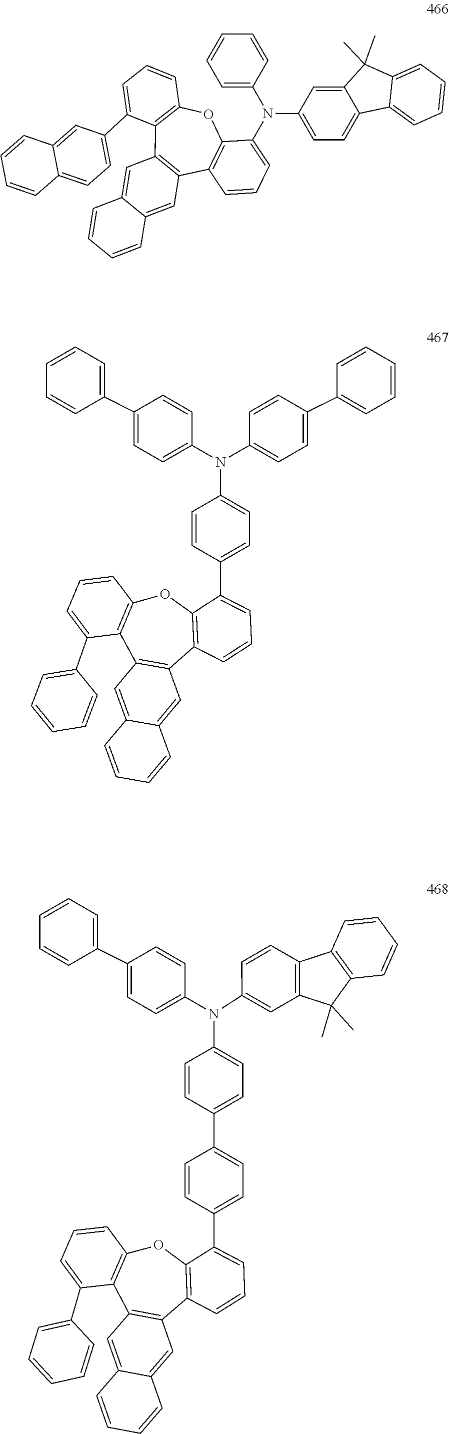 [SMILES: CC1(C)c2ccccc2-c2ccc(N(c3ccccc3)c3cccc4c3Oc3cccc(-c5ccc6ccccc6c5)c3-c3cc5ccccc5cc3-4)cc21 ; c1ccc(-c2ccc(N(c3ccc(-c4ccccc4)cc3)c3ccc(-c4cccc5c4Oc4cccc(-c6ccccc6)c4-c4cc6ccccc6cc4-5)cc3)cc2)cc1 ; CC1(C)c2ccccc2-c2ccc(N(c3ccc(-c4ccccc4)cc3)c3ccc(-c4ccc(-c5cccc6c5Oc5cccc(-c7ccccc7)c5-c5cc7ccccc7cc5-6)cc4)cc3)cc21]

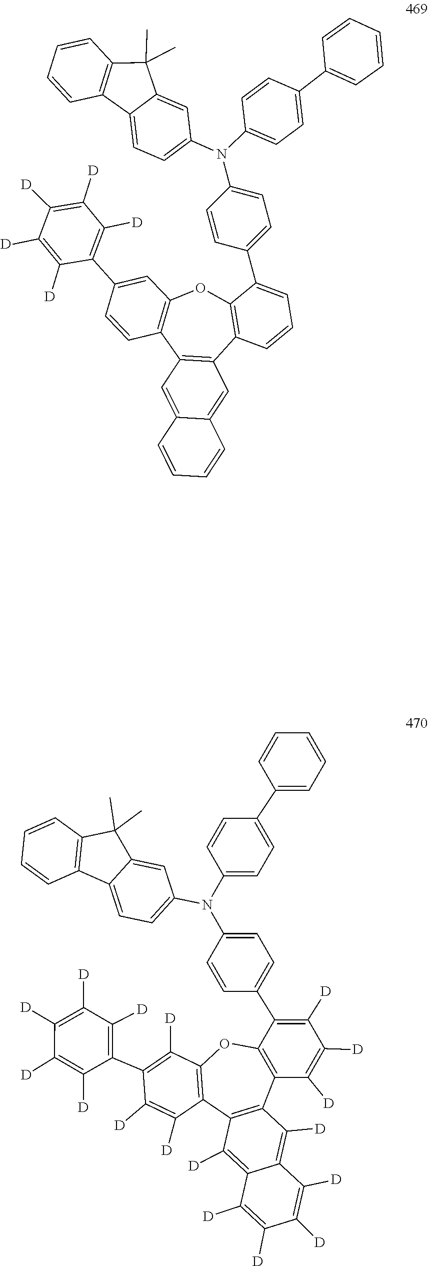 [SMILES: [2H]c1c([2H])c([2H])c(-c2ccc3c(c2)Oc2c(-c4ccc(N(c5ccc(-c6ccccc6)cc5)c5ccc6c(c5)C(C)(C)c5ccccc5-6)cc4)cccc2-c2cc4ccccc4cc2-3)c([2H])c1[2H] ; [2H]c1c([2H])c([2H])c(-c2c([2H])c([2H])c3c(c2[2H])Oc2c(-c4ccc(N(c5ccc(-c6ccccc6)cc5)c5ccc6c(c5)C(C)(C)c5ccccc5-6)cc4)c([2H])c([2H])c([2H])c2-c2c-3c([2H])c3c([2H])c([2H])c([2H])c([2H])c3c2[2H])c([2H])c1[2H]]

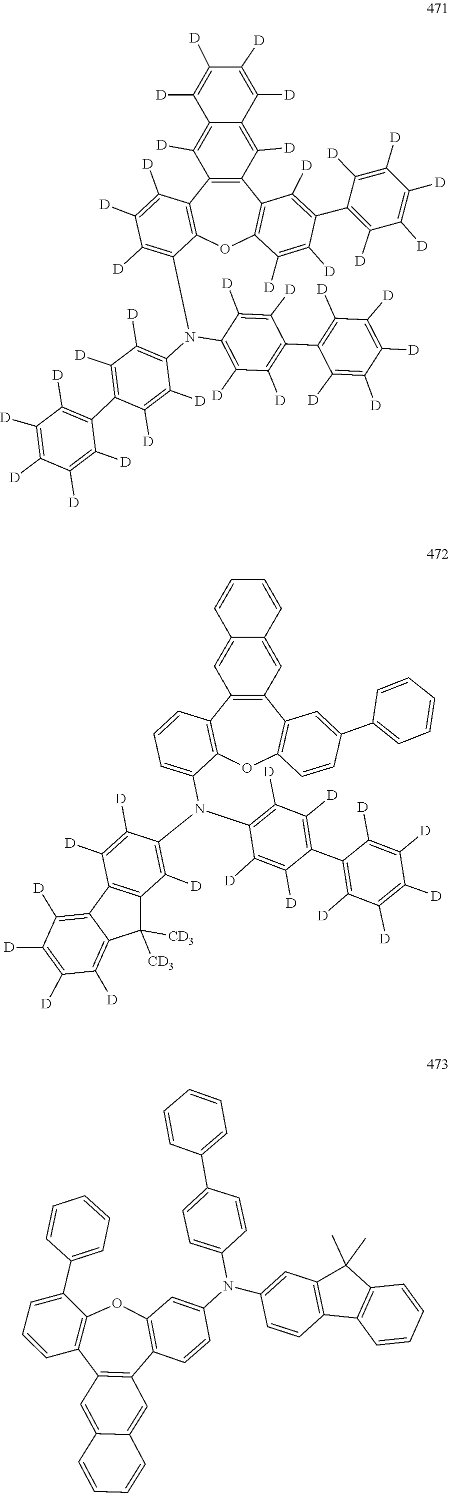

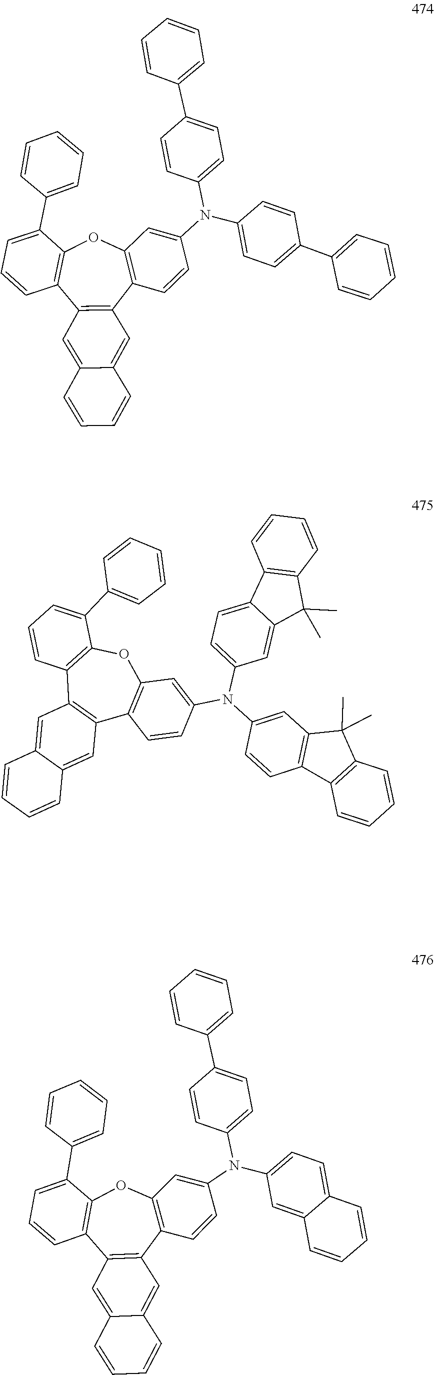 [SMILES: c1ccc(-c2ccc(N(c3ccc(-c4ccccc4)cc3)c3ccc4c(c3)Oc3c(-c5ccccc5)cccc3-c3cc5ccccc5cc3-4)cc2)cc1 ; CC1(C)c2ccccc2-c2ccc(N(c3ccc4c(c3)Oc3c(-c5ccccc5)cccc3-c3cc5ccccc5cc3-4)c3ccc4c(c3)C(C)(C)c3ccccc3-4)cc21 ; c1ccc(-c2ccc(N(c3ccc4c(c3)Oc3c(-c5ccccc5)cccc3-c3cc5ccccc5cc3-4)c3ccc4ccccc4c3)cc2)cc1]

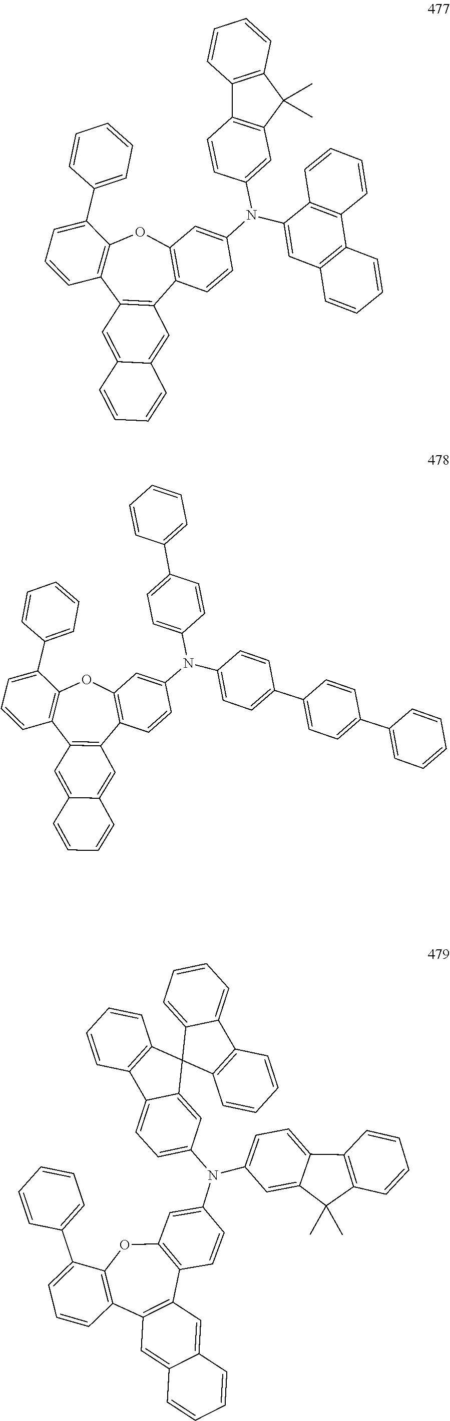 [SMILES: CC1(C)c2ccccc2-c2ccc(N(c3ccc4c(c3)Oc3c(-c5ccccc5)cccc3-c3cc5ccccc5cc3-4)c3cc4ccccc4c4ccccc34)cc21 ; c1ccc(-c2ccc(-c3ccc(N(c4ccc(-c5ccccc5)cc4)c4ccc5c(c4)Oc4c(-c6ccccc6)cccc4-c4cc6ccccc6cc4-5)cc3)cc2)cc1 ; CC1(C)c2ccccc2-c2ccc(N(c3ccc4c(c3)Oc3c(-c5ccccc5)cccc3-c3cc5ccccc5cc3-4)c3ccc4c(c3)C3(c5ccccc5-c5ccccc53)c3ccccc3-4)cc21]

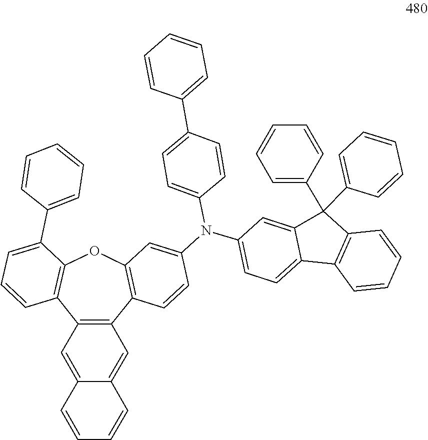 [SMILES: c1ccc(-c2ccc(N(c3ccc4c(c3)Oc3c(-c5ccccc5)cccc3-c3cc5ccccc5cc3-4)c3ccc4c(c3)C(c3ccccc3)(c3ccccc3)c3ccccc3-4)cc2)cc1]

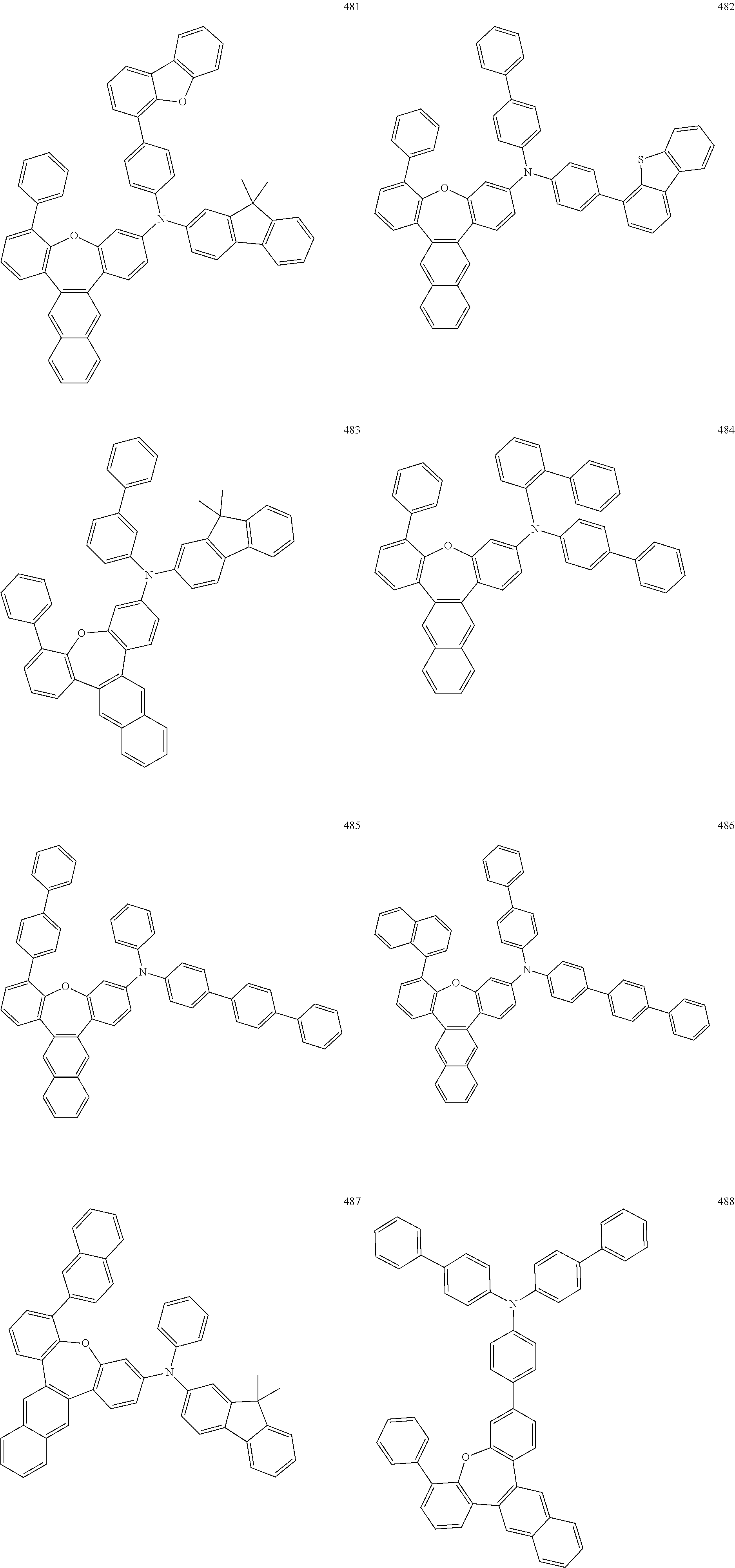

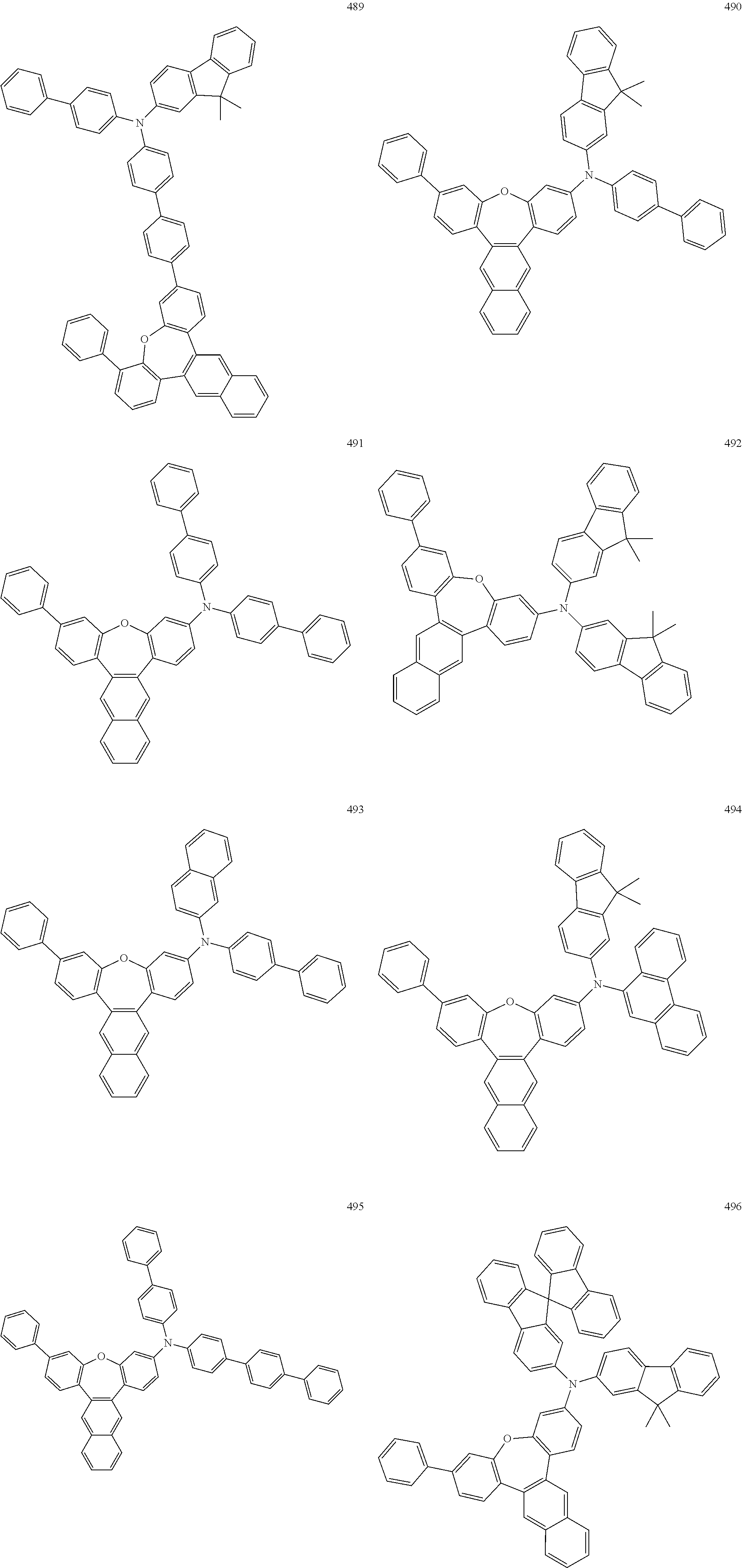

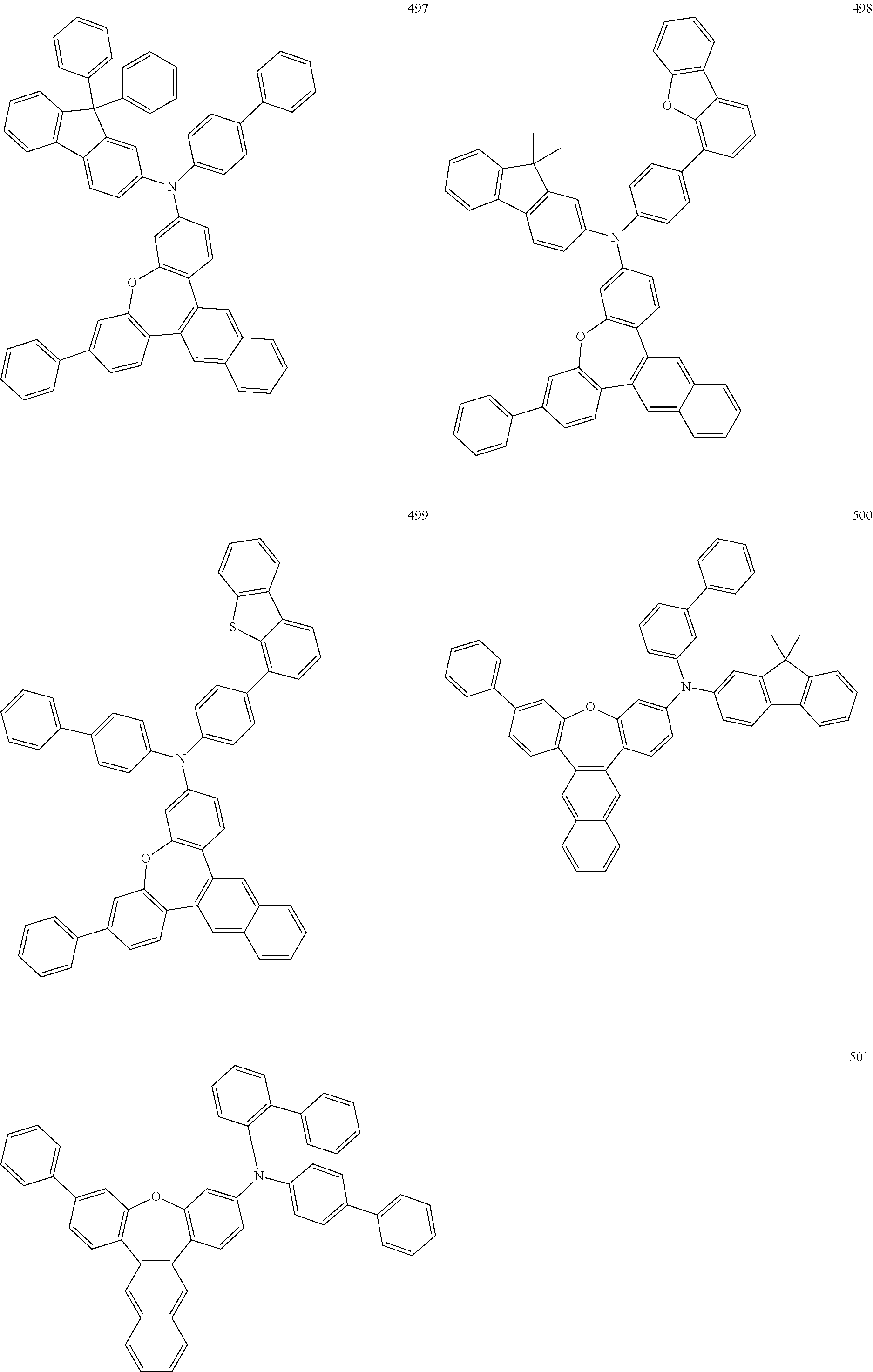

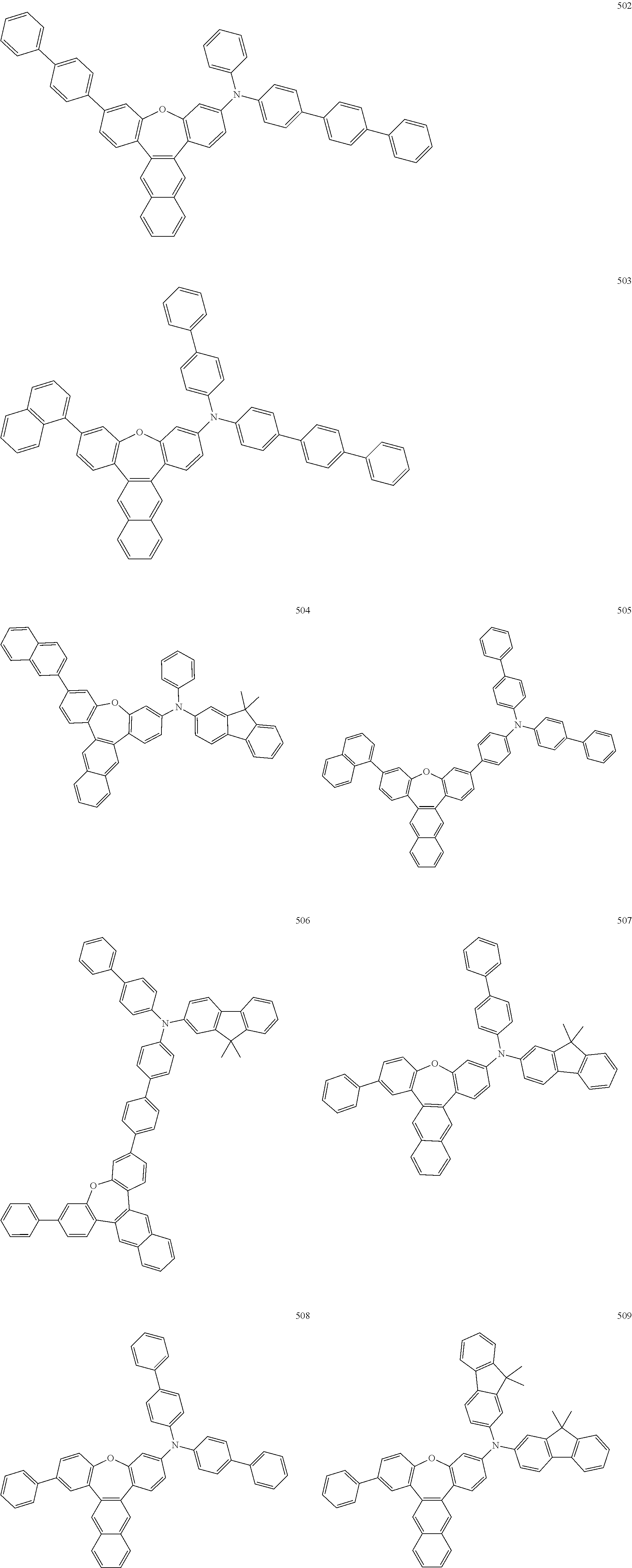

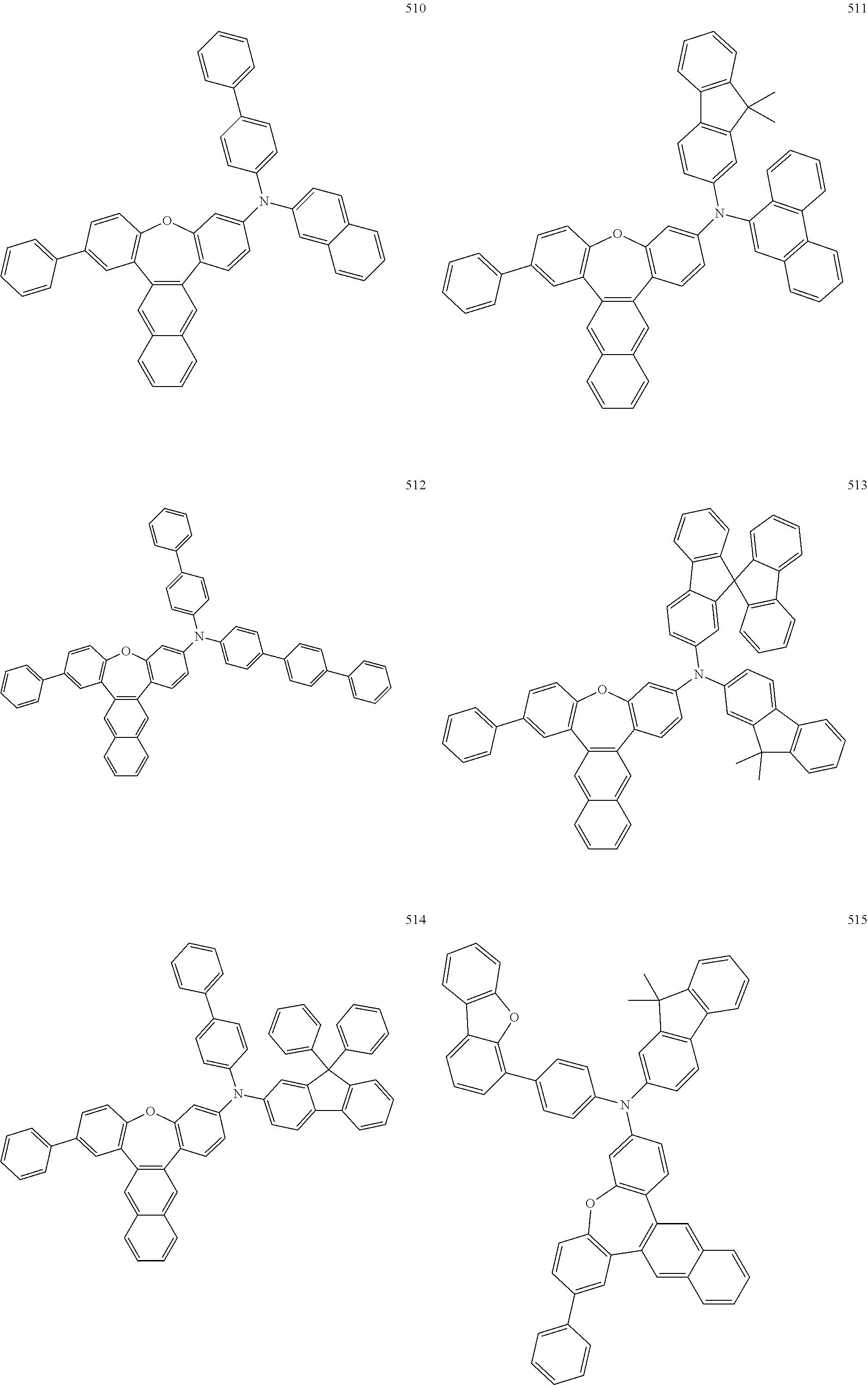

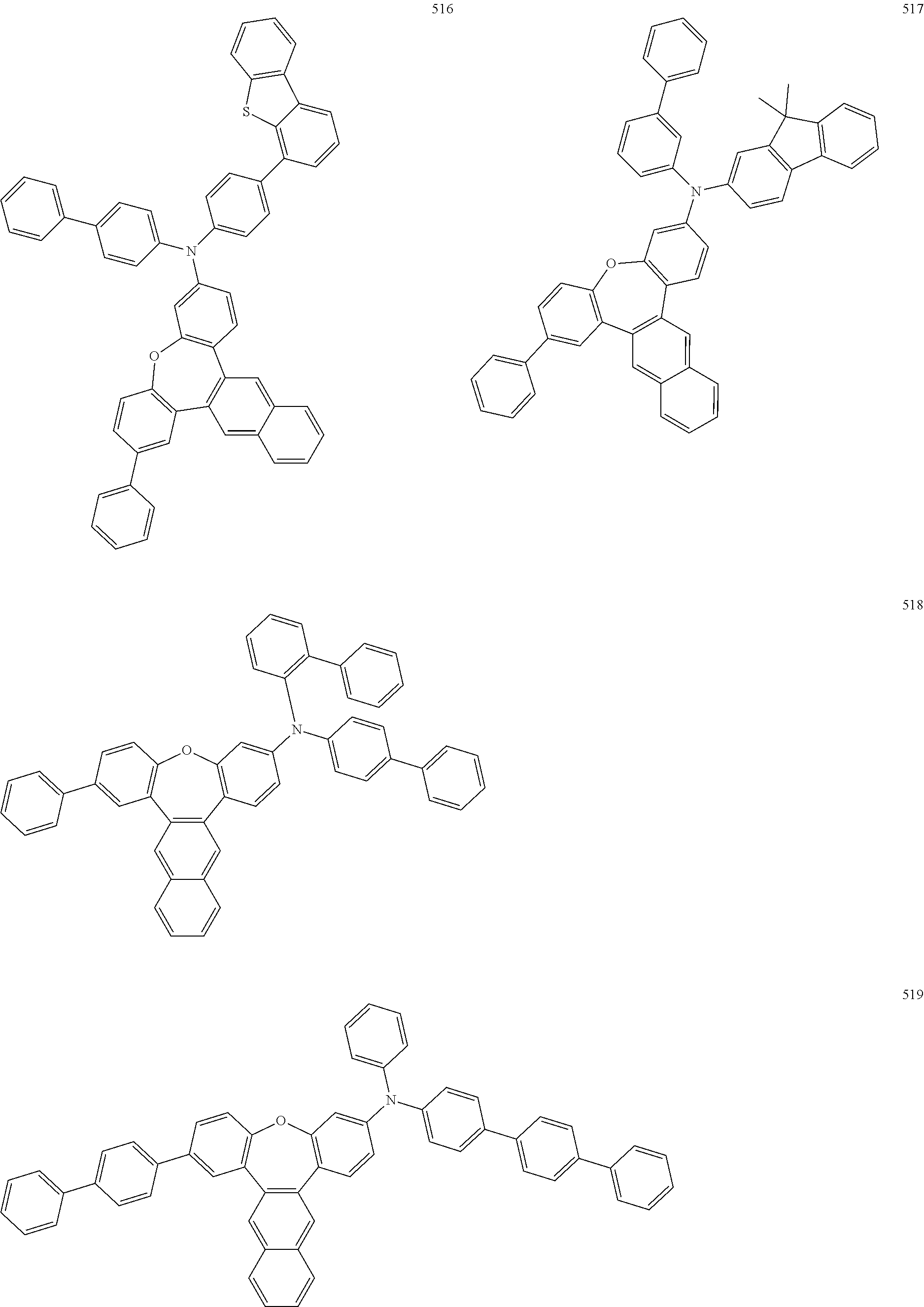 [SMILES: c1ccc(-c2ccc(N(c3ccc(-c4cccc5c4sc4ccccc45)cc3)c3ccc4c(c3)Oc3ccc(-c5ccccc5)cc3-c3cc5ccccc5cc3-4)cc2)cc1 ; CC1(C)c2ccccc2-c2ccc(N(c3cccc(-c4ccccc4)c3)c3ccc4c(c3)Oc3ccc(-c5ccccc5)cc3-c3cc5ccccc5cc3-4)cc21 ; c1ccc(-c2ccc(N(c3ccc4c(c3)Oc3ccc(-c5ccccc5)cc3-c3cc5ccccc5cc3-4)c3ccccc3-c3ccccc3)cc2)cc1 ; c1ccc(-c2ccc(-c3ccc(N(c4ccccc4)c4ccc5c(c4)Oc4ccc(-c6ccc(-c7ccccc7)cc6)cc4-c4cc6ccccc6cc4-5)cc3)cc2)cc1]

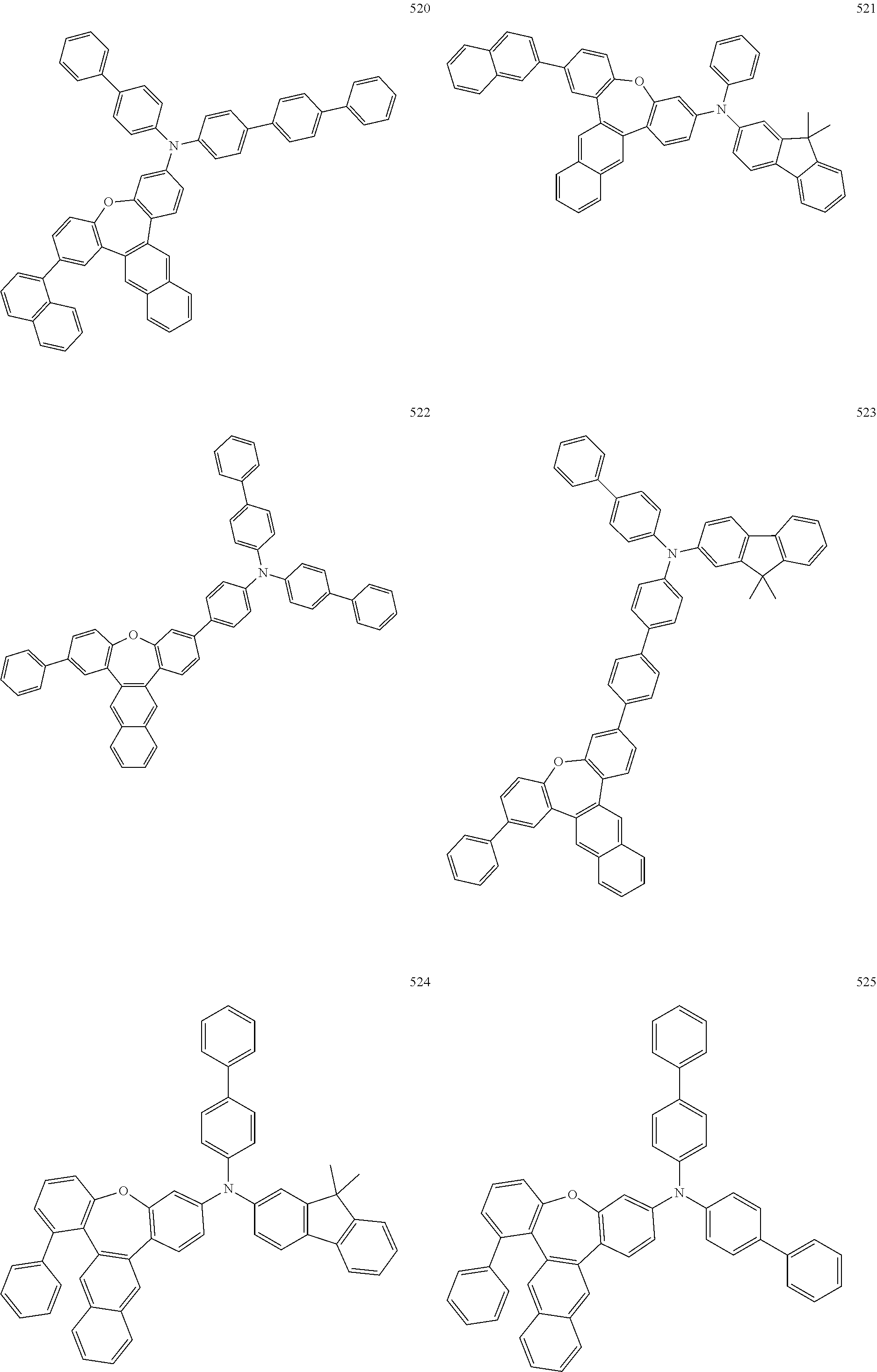

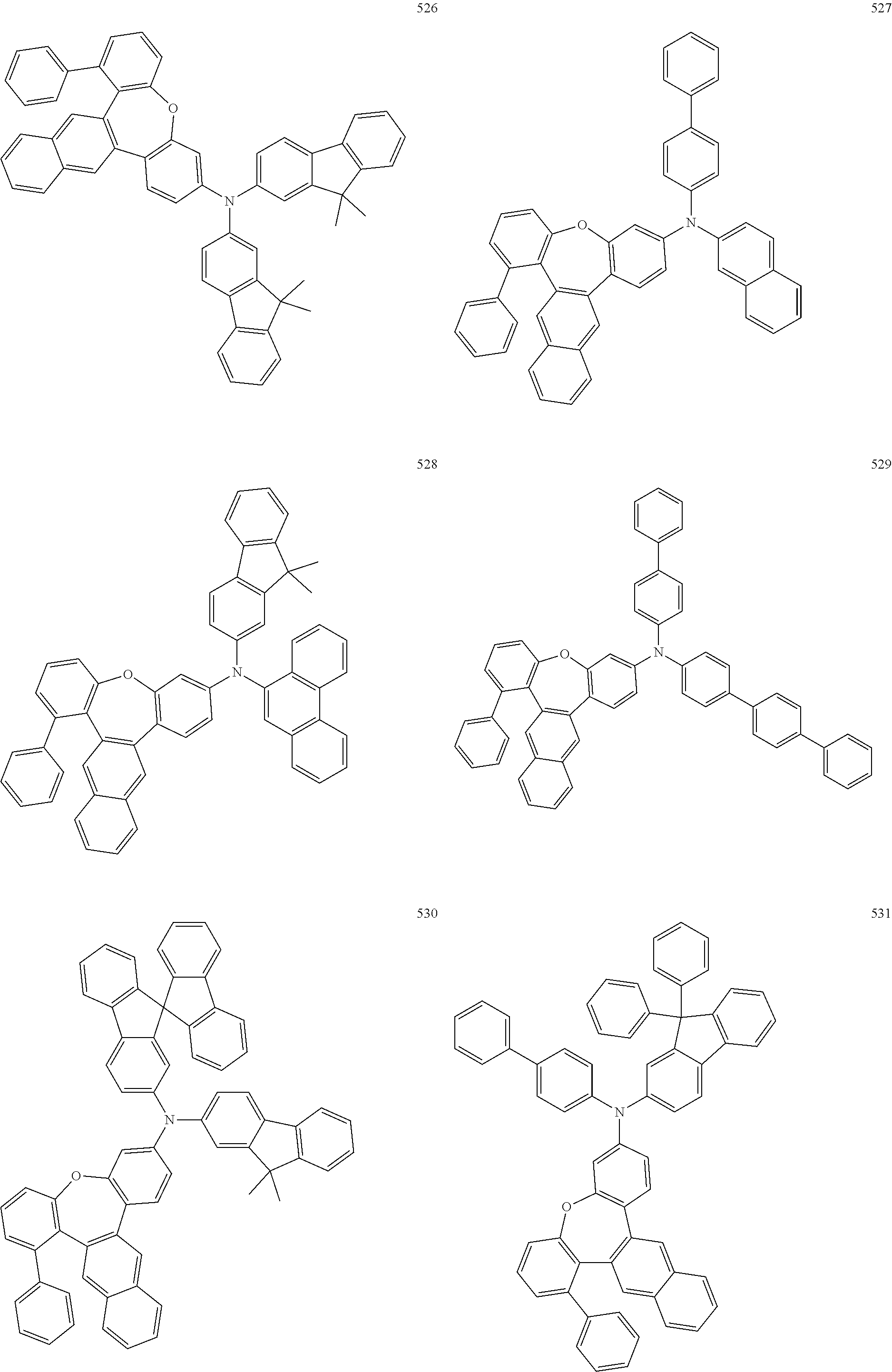

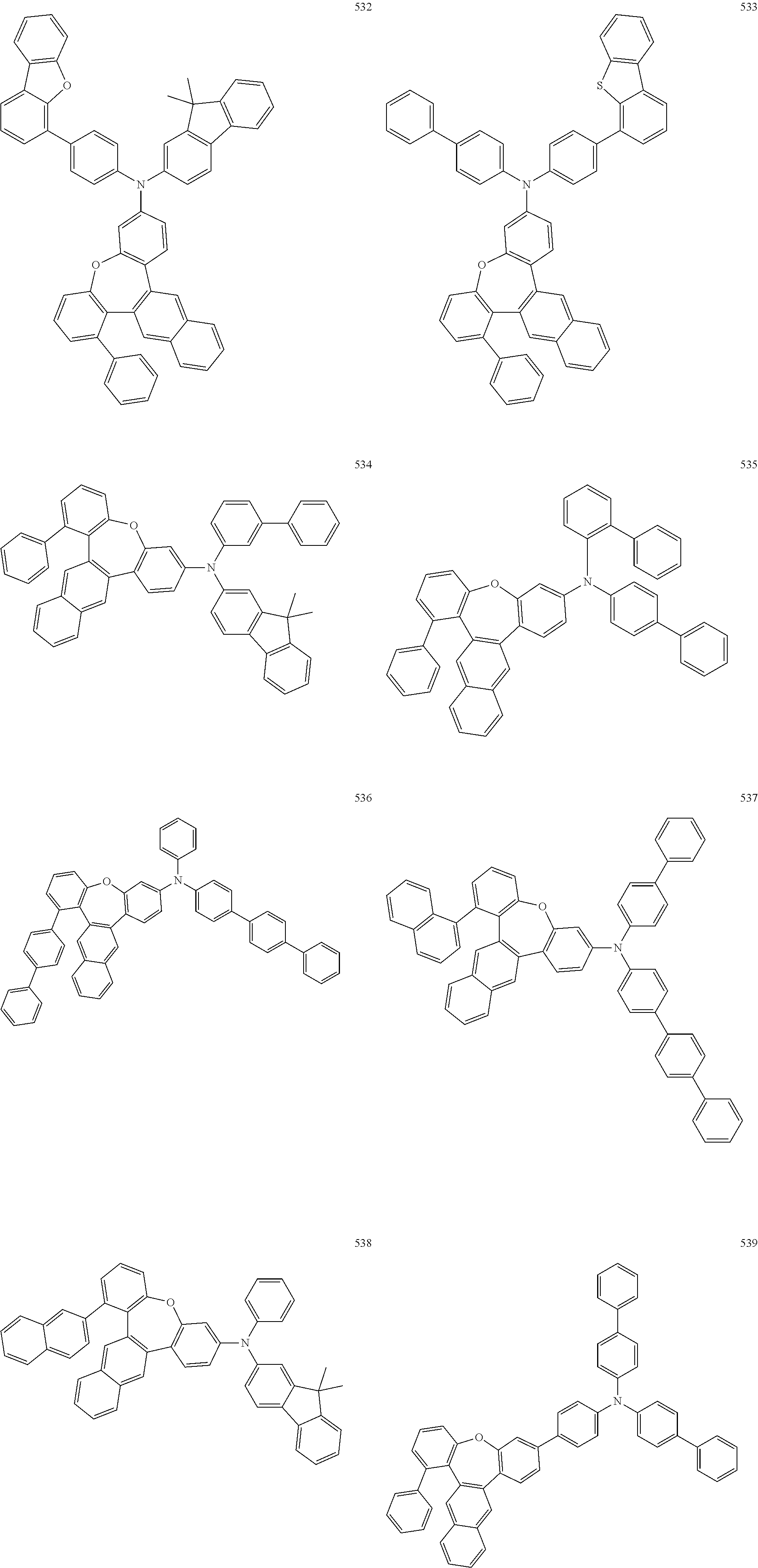

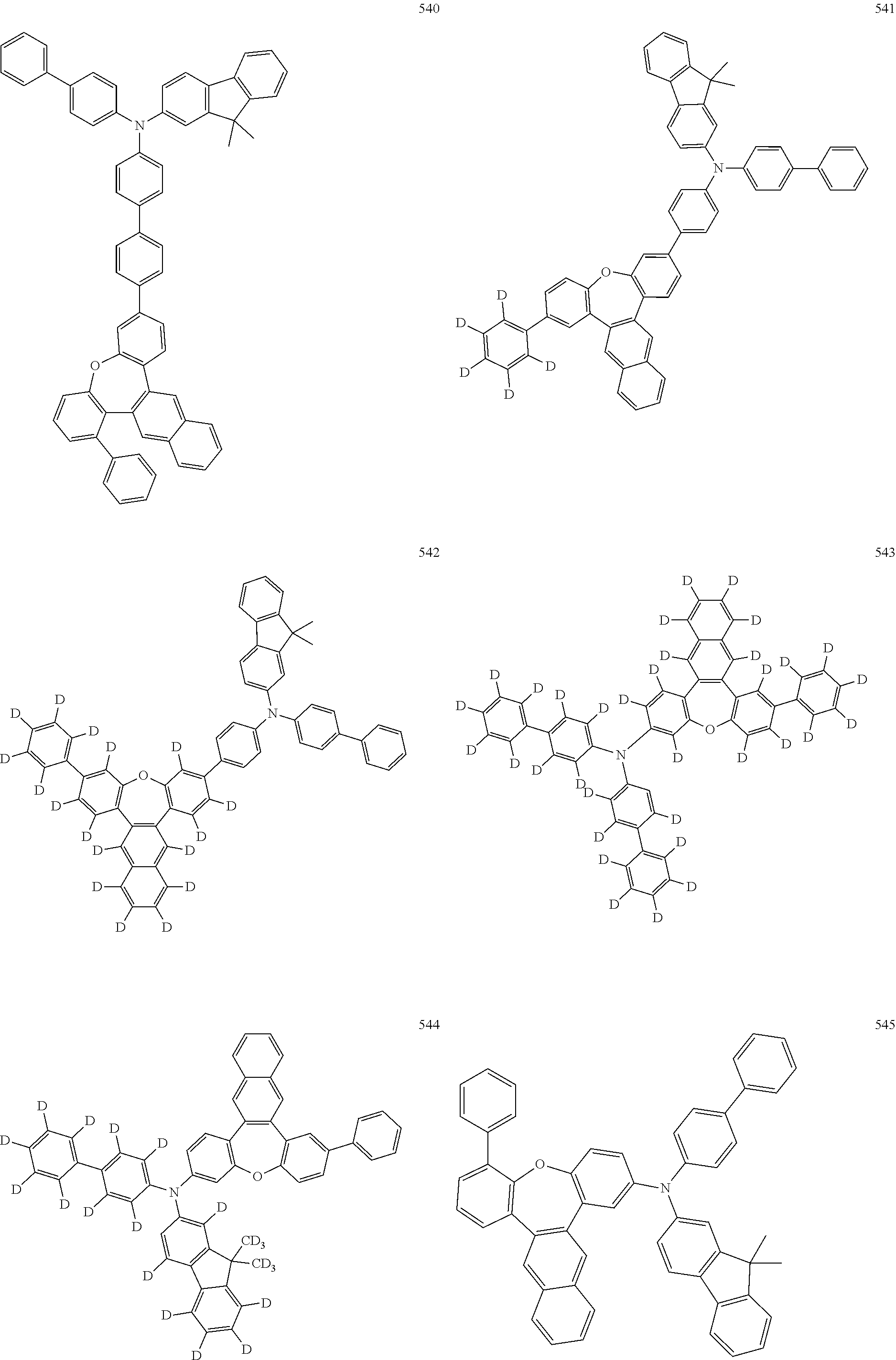

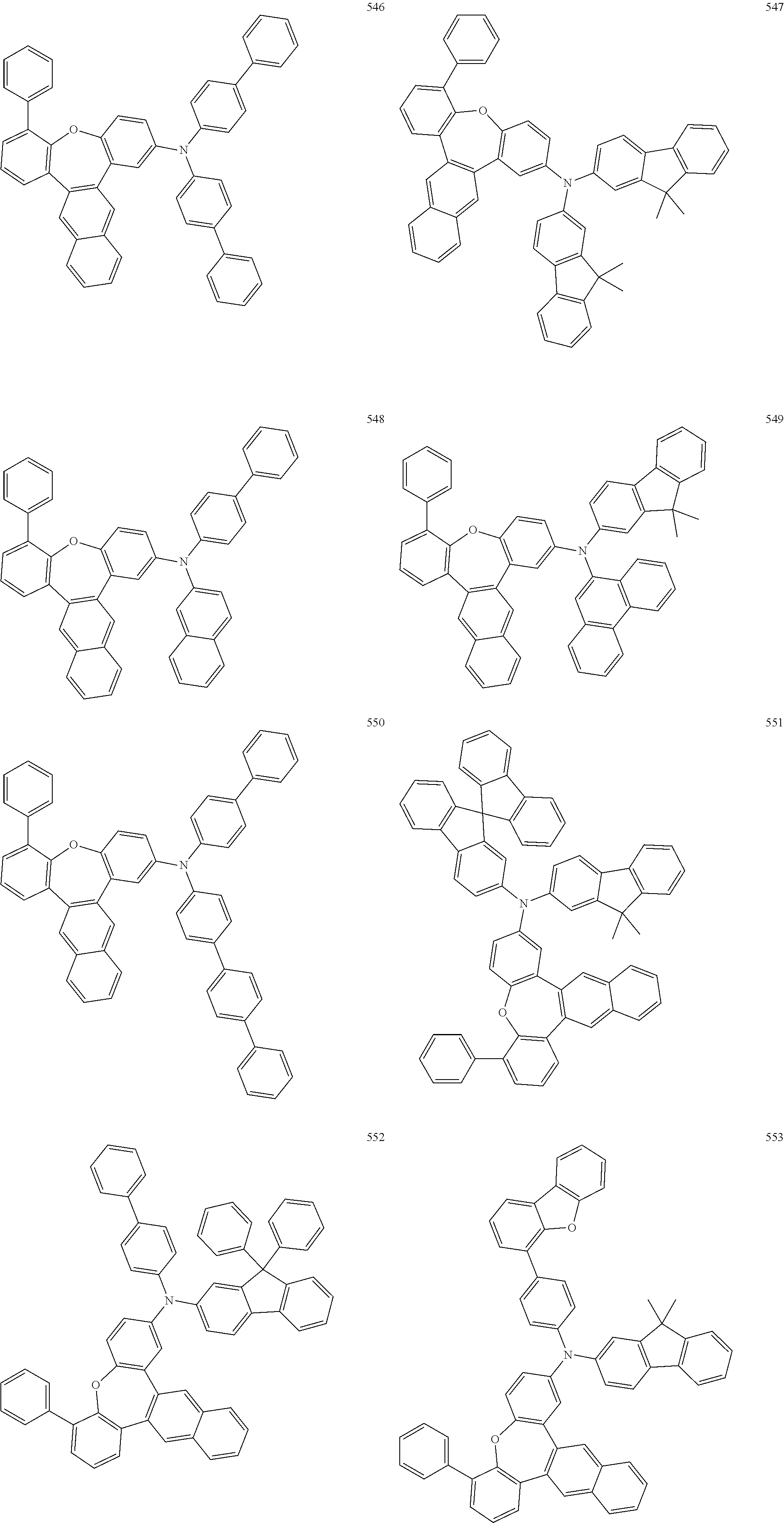

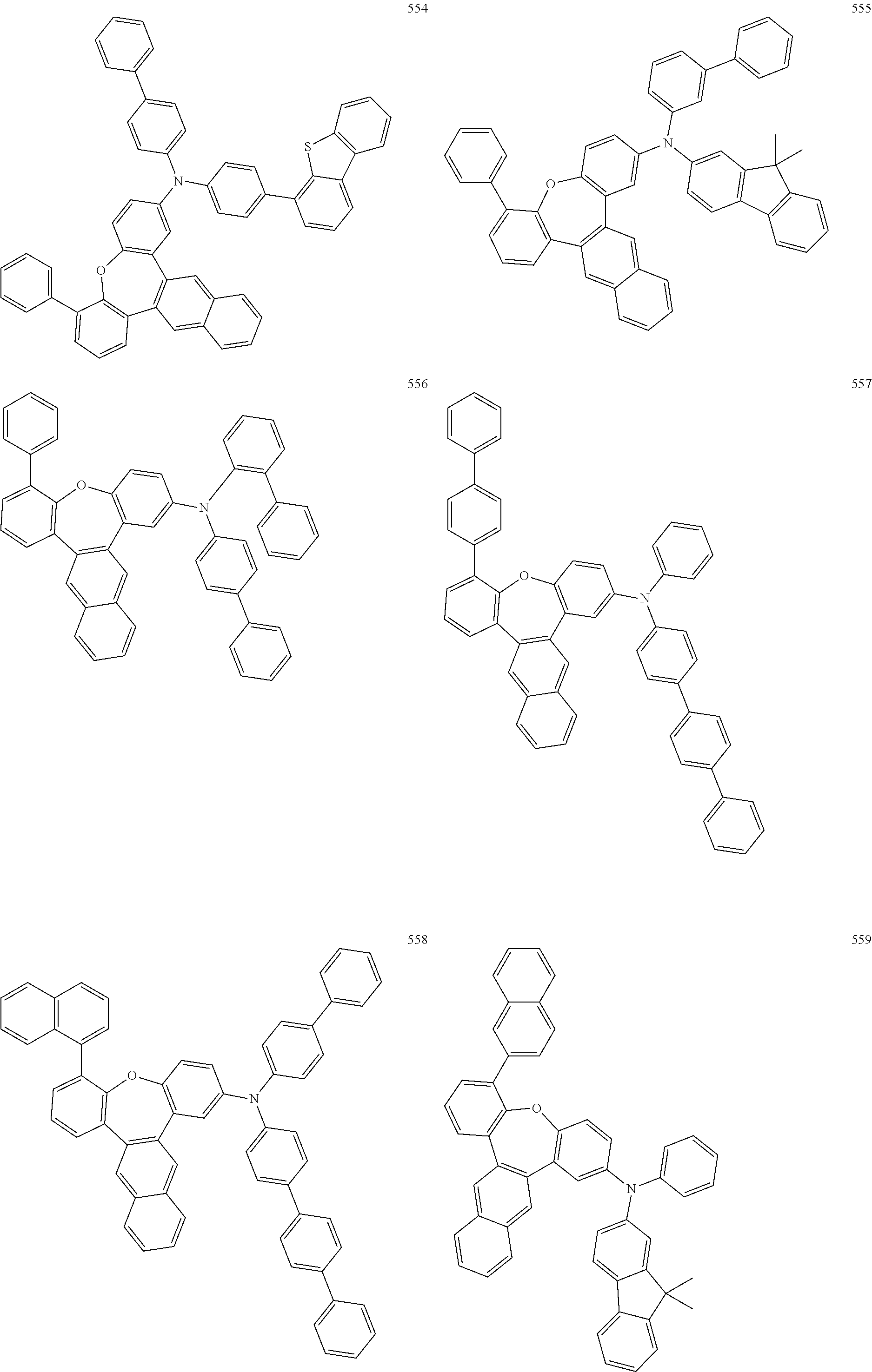

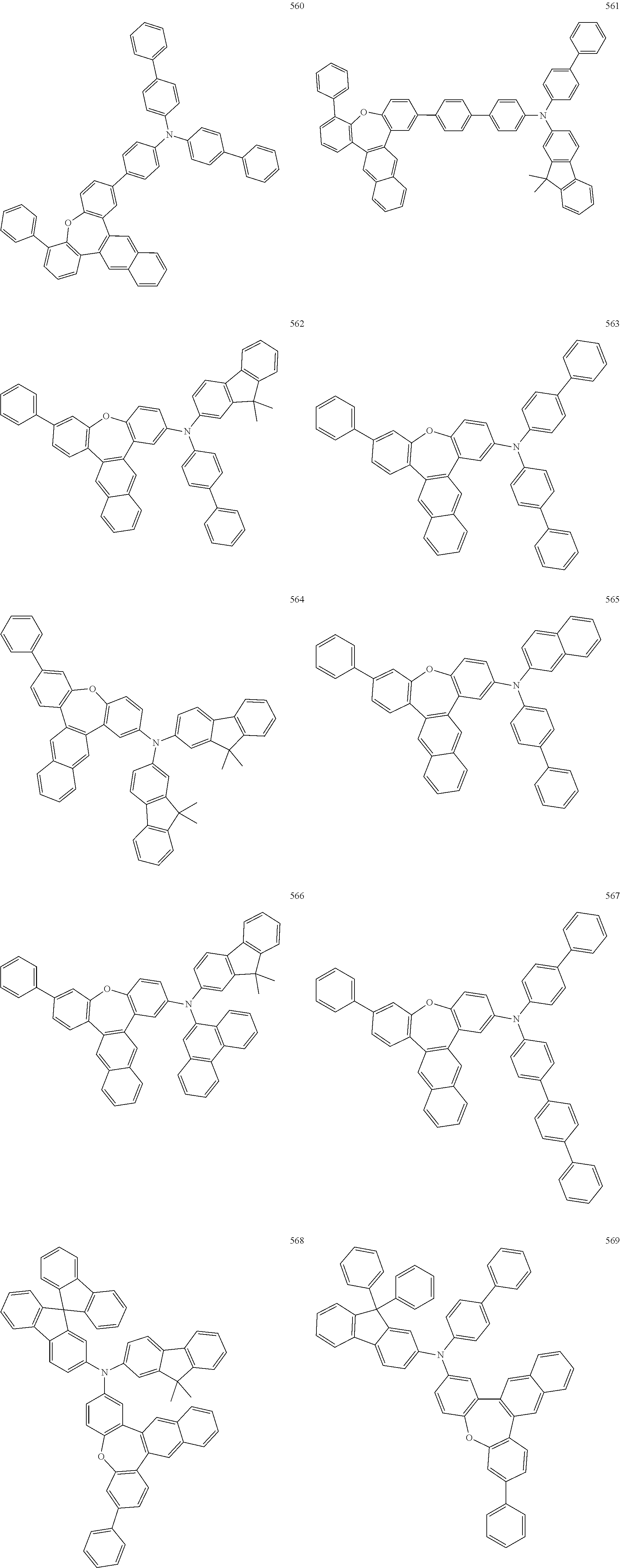

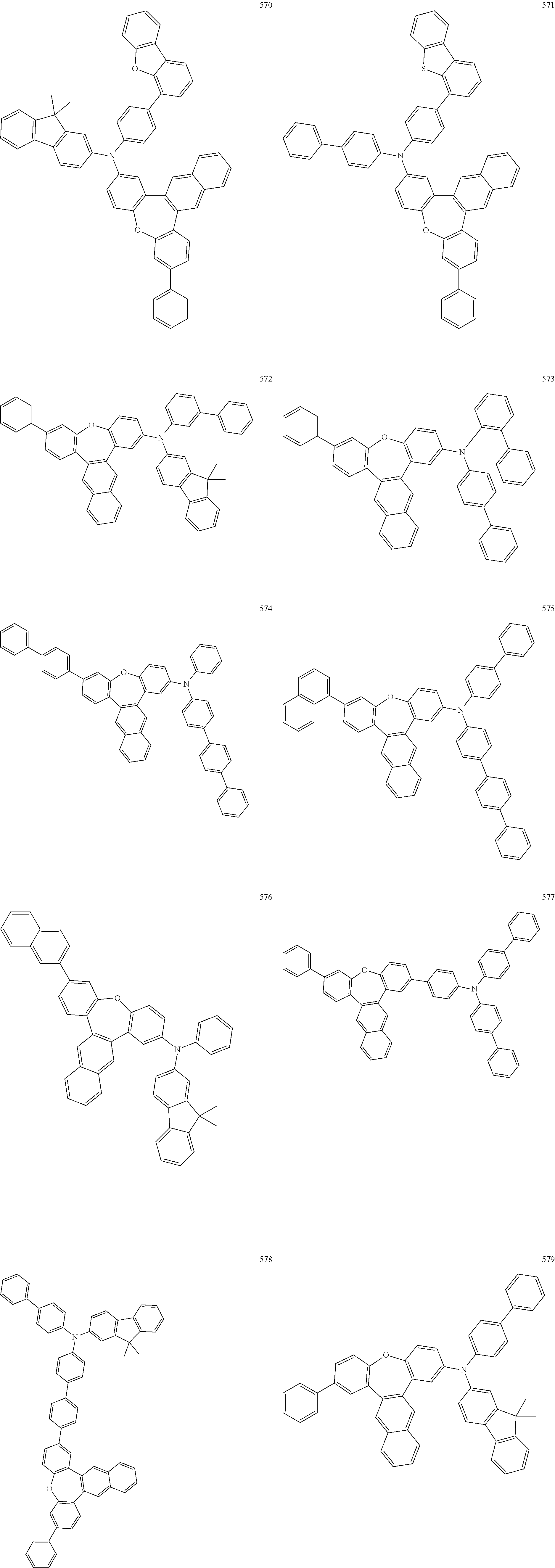

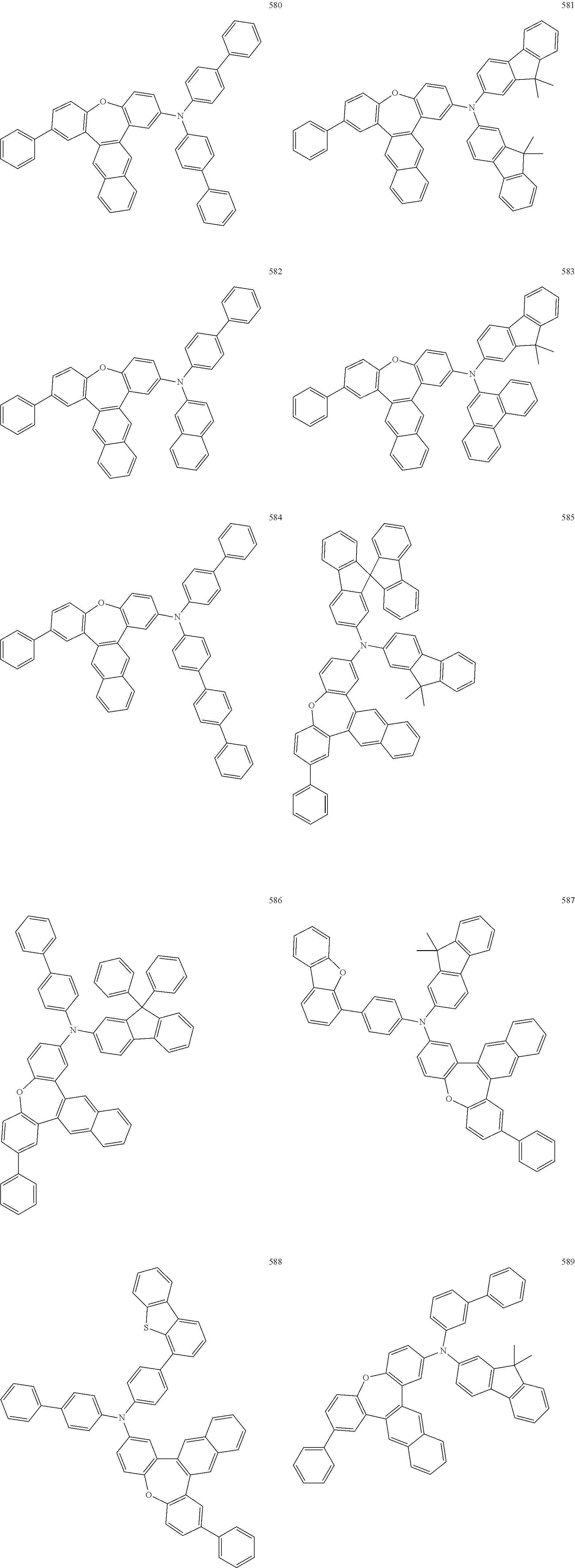

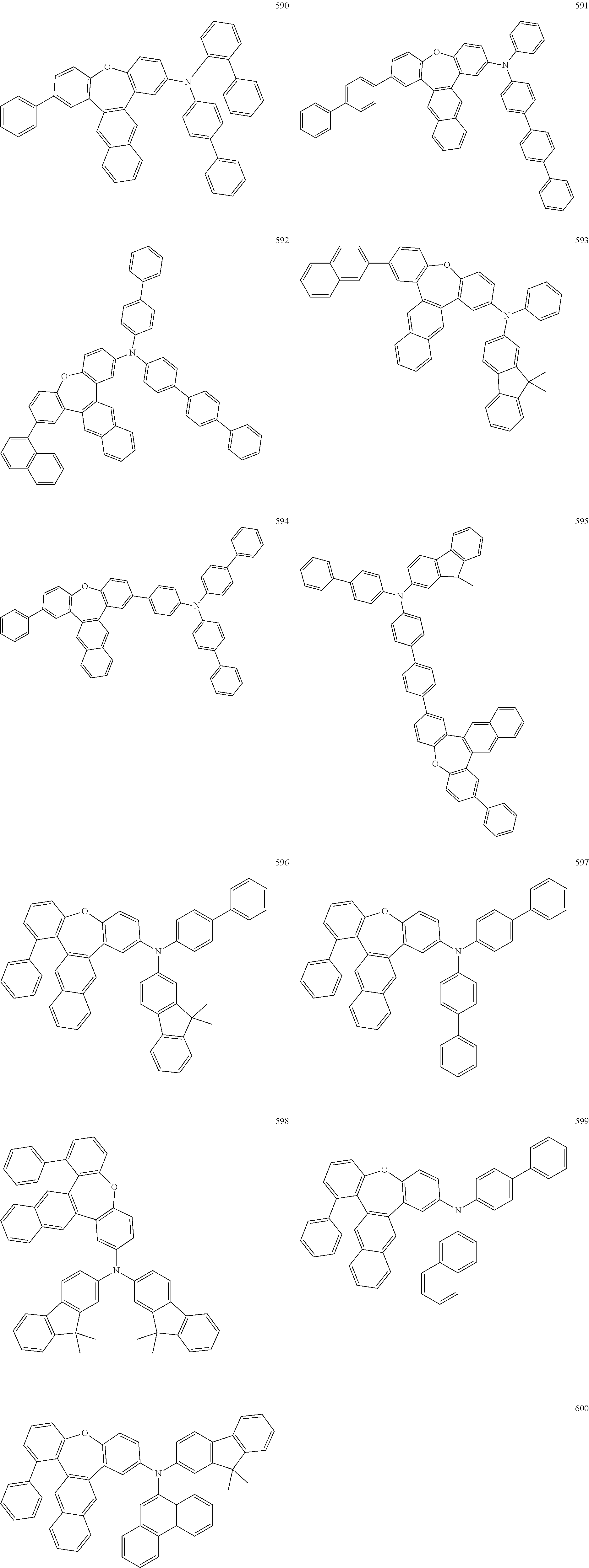

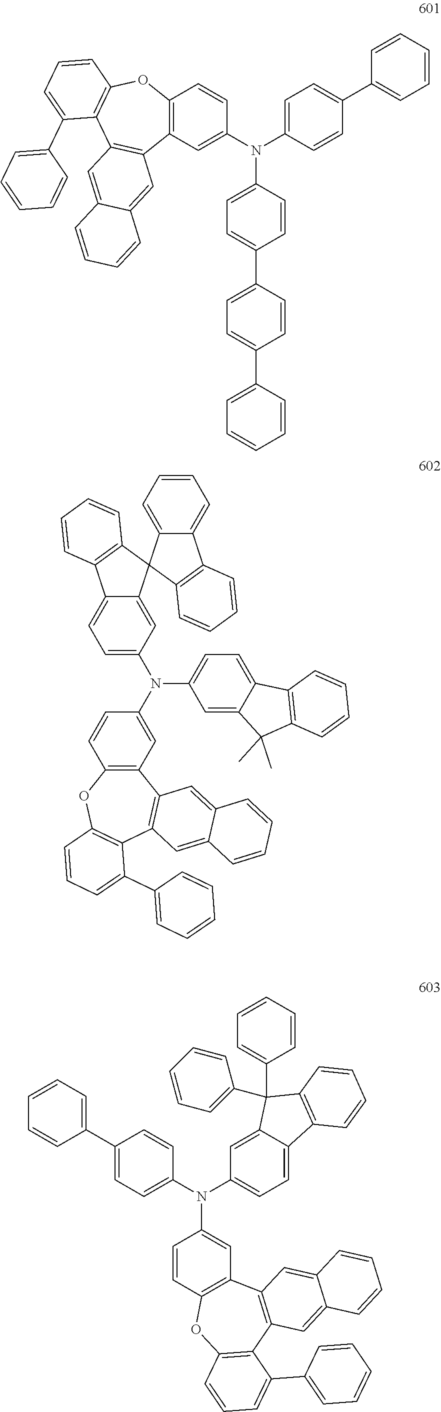 [SMILES: c1ccc(-c2ccc(-c3ccc(N(c4ccc(-c5ccccc5)cc4)c4ccc5c(c4)-c4cc6ccccc6cc4-c4c(cccc4-c4ccccc4)O5)cc3)cc2)cc1 ; CC1(C)c2ccccc2-c2ccc(N(c3ccc4c(c3)-c3cc5ccccc5cc3-c3c(cccc3-c3ccccc3)O4)c3ccc4c(c3)C3(c5ccccc5-c5ccccc53)c3ccccc3-4)cc21 ; c1ccc(-c2ccc(N(c3ccc4c(c3)-c3cc5ccccc5cc3-c3c(cccc3-c3ccccc3)O4)c3ccc4c(c3)C(c3ccccc3)(c3ccccc3)c3ccccc3-4)cc2)cc1]

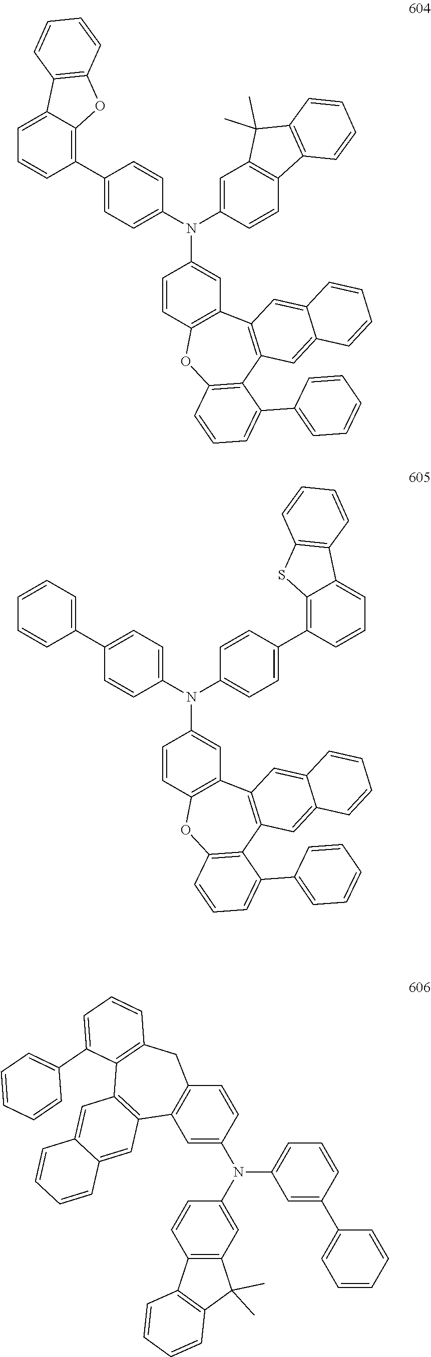 [SMILES: CC1(C)c2ccccc2-c2ccc(N(c3ccc(-c4cccc5c4oc4ccccc45)cc3)c3ccc4c(c3)-c3cc5ccccc5cc3-c3c(cccc3-c3ccccc3)O4)cc21 ; c1ccc(-c2ccc(N(c3ccc(-c4cccc5c4sc4ccccc45)cc3)c3ccc4c(c3)-c3cc5ccccc5cc3-c3c(cccc3-c3ccccc3)O4)cc2)cc1 ; CC1(C)c2ccccc2-c2ccc(N(c3cccc(-c4ccccc4)c3)c3ccc4c(c3)-c3cc5ccccc5cc3-c3c(cccc3-c3ccccc3)C4)cc21]

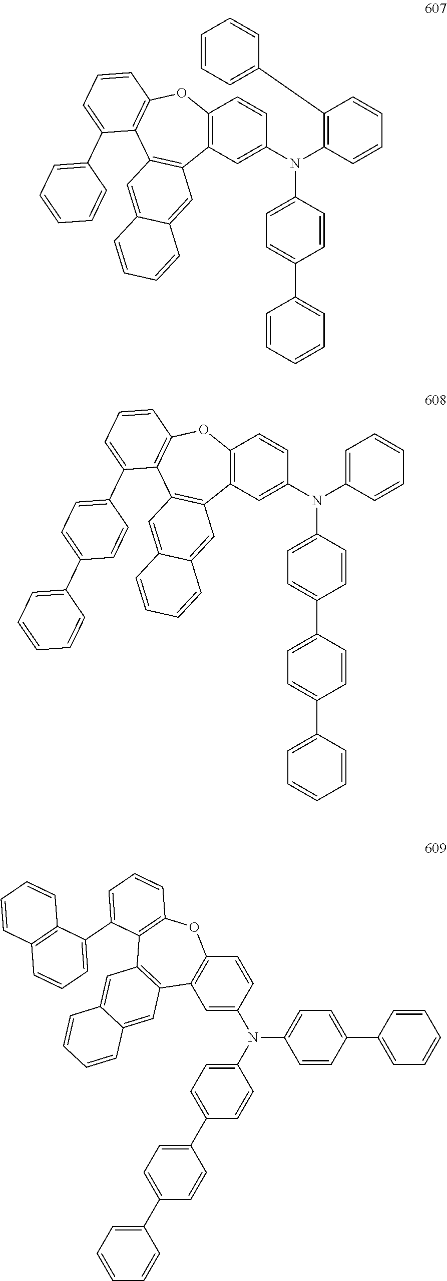 [SMILES: c1ccc(-c2ccc(N(c3ccc4c(c3)-c3cc5ccccc5cc3-c3c(cccc3-c3ccccc3)O4)c3ccccc3-c3ccccc3)cc2)cc1 ; c1ccc(-c2ccc(-c3ccc(N(c4ccccc4)c4ccc5c(c4)-c4cc6ccccc6cc4-c4c(cccc4-c4ccc(-c6ccccc6)cc4)O5)cc3)cc2)cc1 ; c1ccc(-c2ccc(-c3ccc(N(c4ccc(-c5ccccc5)cc4)c4ccc5c(c4)-c4cc6ccccc6cc4-c4c(cccc4-c4cccc6ccccc46)O5)cc3)cc2)cc1]

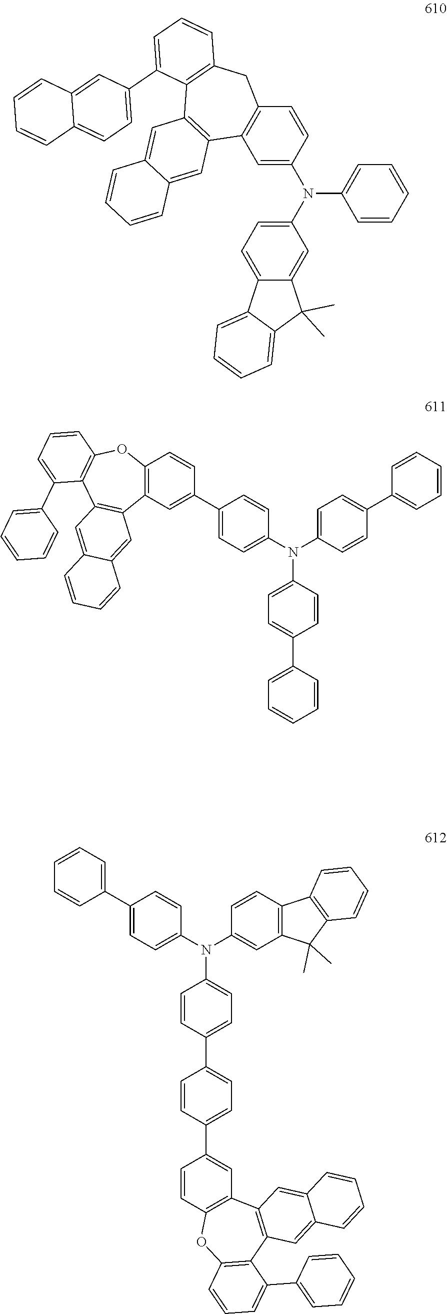 [SMILES: CC1(C)c2ccccc2-c2ccc(N(c3ccccc3)c3ccc4c(c3)-c3cc5ccccc5cc3-c3c(cccc3-c3ccc5ccccc5c3)C4)cc21 ; c1ccc(-c2ccc(N(c3ccc(-c4ccccc4)cc3)c3ccc(-c4ccc5c(c4)-c4cc6ccccc6cc4-c4c(cccc4-c4ccccc4)O5)cc3)cc2)cc1 ; CC1(C)c2ccccc2-c2ccc(N(c3ccc(-c4ccccc4)cc3)c3ccc(-c4ccc(-c5ccc6c(c5)-c5cc7ccccc7cc5-c5c(cccc5-c5ccccc5)O6)cc4)cc3)cc21]

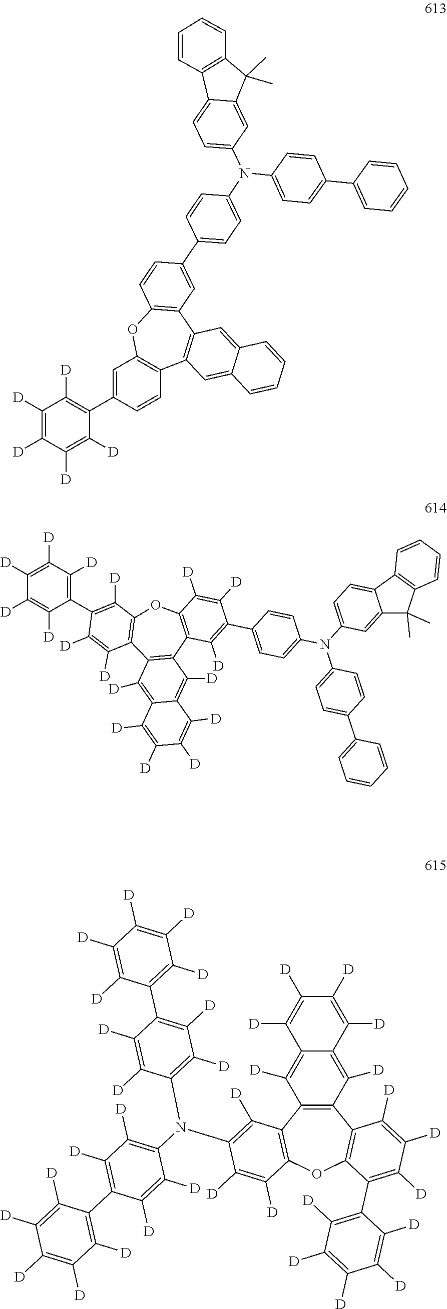

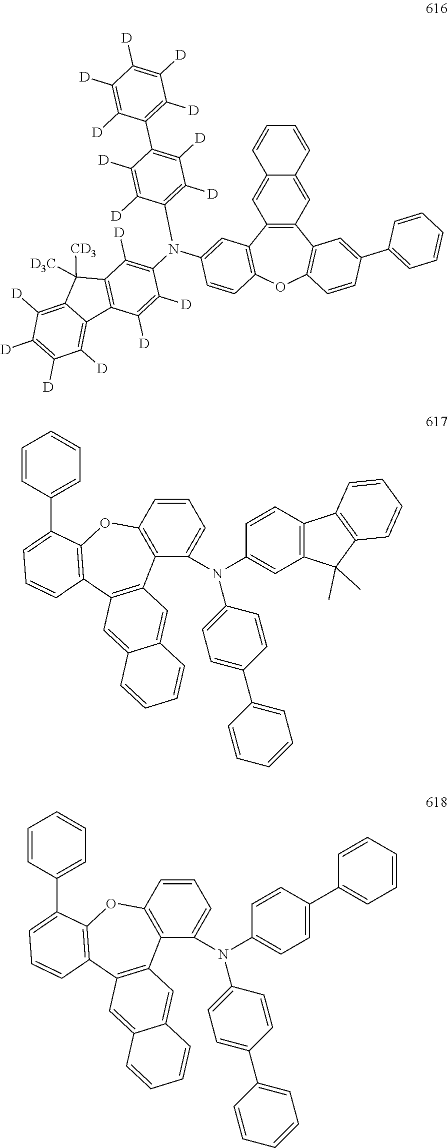

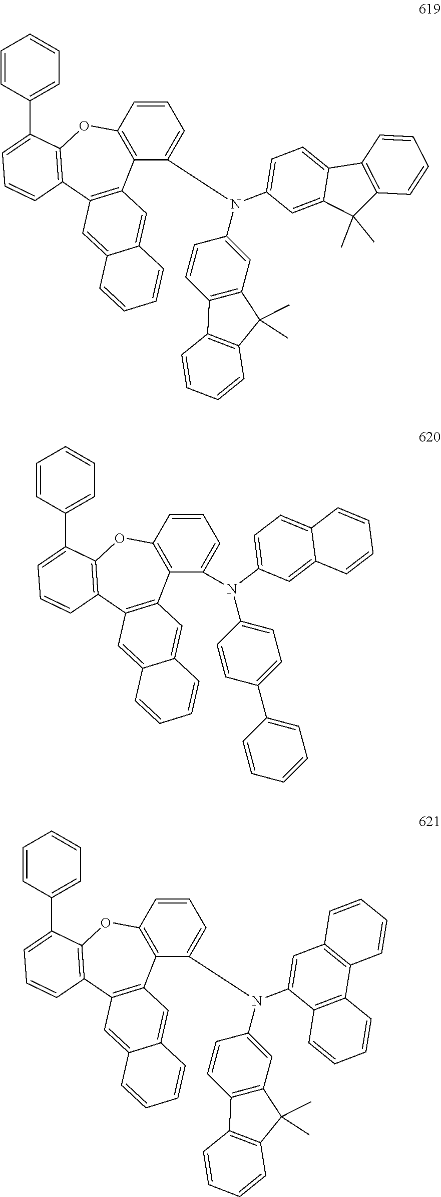 [SMILES: CC1(C)c2ccccc2-c2ccc(N(c3ccc4c(c3)C(C)(C)c3ccccc3-4)c3cccc4c3-c3cc5ccccc5cc3-c3cccc(-c5ccccc5)c3O4)cc21 ; c1ccc(-c2ccc(N(c3ccc4ccccc4c3)c3cccc4c3-c3cc5ccccc5cc3-c3cccc(-c5ccccc5)c3O4)cc2)cc1 ; CC1(C)c2ccccc2-c2ccc(N(c3cccc4c3-c3cc5ccccc5cc3-c3cccc(-c5ccccc5)c3O4)c3cc4ccccc4c4ccccc34)cc21]

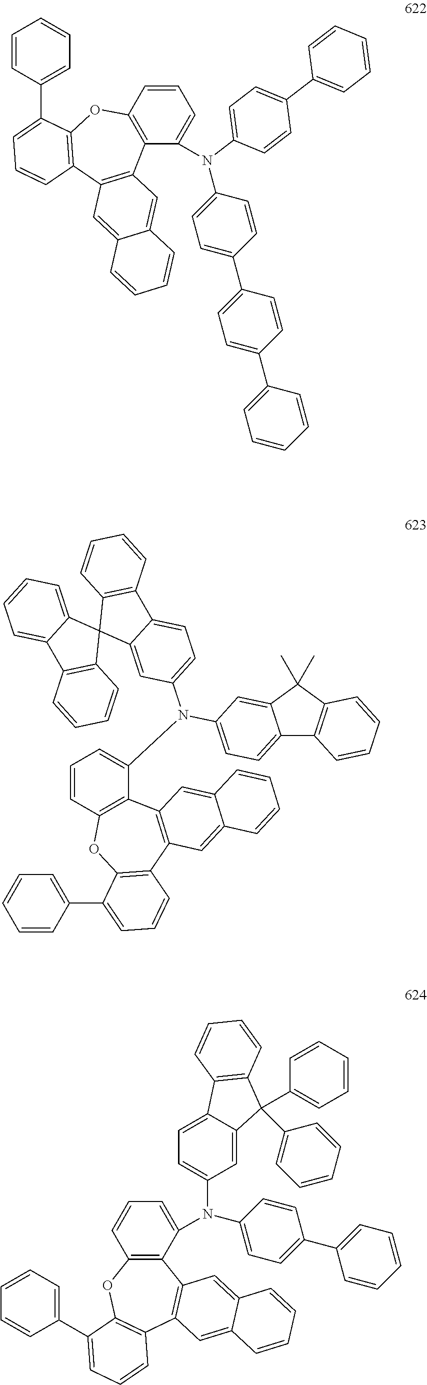 [SMILES: c1ccc(-c2ccc(-c3ccc(N(c4ccc(-c5ccccc5)cc4)c4cccc5c4-c4cc6ccccc6cc4-c4cccc(-c6ccccc6)c4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3cc5ccccc5cc3-c3cccc(-c5ccccc5)c3O4)cc21 ; c1ccc(-c2ccc(N(c3ccc4c(c3)C(c3ccccc3)(c3ccccc3)c3ccccc3-4)c3cccc4c3-c3cc5ccccc5cc3-c3cccc(-c5ccccc5)c3O4)cc2)cc1]

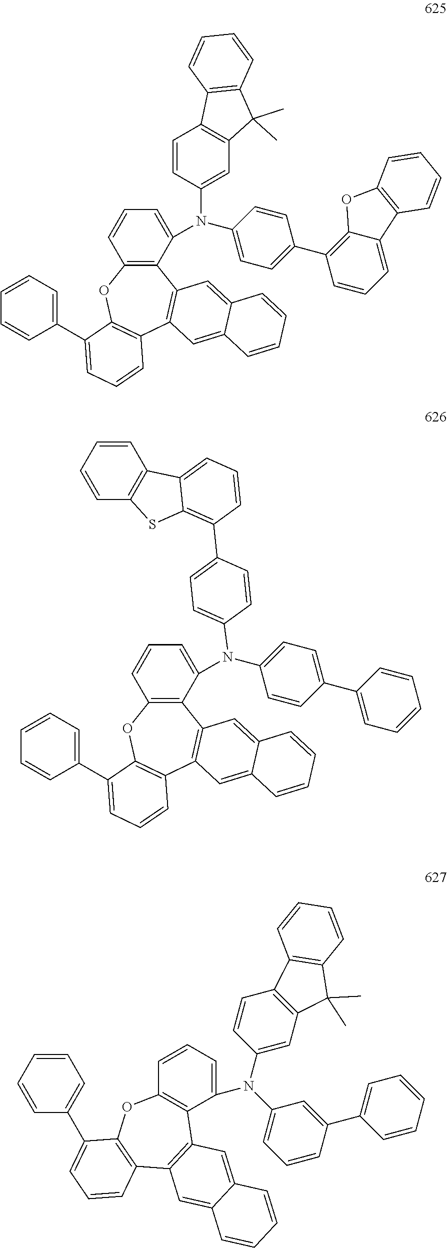 [SMILES: CC1(C)c2ccccc2-c2ccc(N(c3ccc(-c4cccc5c4oc4ccccc45)cc3)c3cccc4c3-c3cc5ccccc5cc3-c3cccc(-c5ccccc5)c3O4)cc21 ; c1ccc(-c2ccc(N(c3ccc(-c4cccc5c4sc4ccccc45)cc3)c3cccc4c3-c3cc5ccccc5cc3-c3cccc(-c5ccccc5)c3O4)cc2)cc1 ; CC1(C)c2ccccc2-c2ccc(N(c3cccc(-c4ccccc4)c3)c3cccc4c3-c3cc5ccccc5cc3-c3cccc(-c5ccccc5)c3O4)cc21]

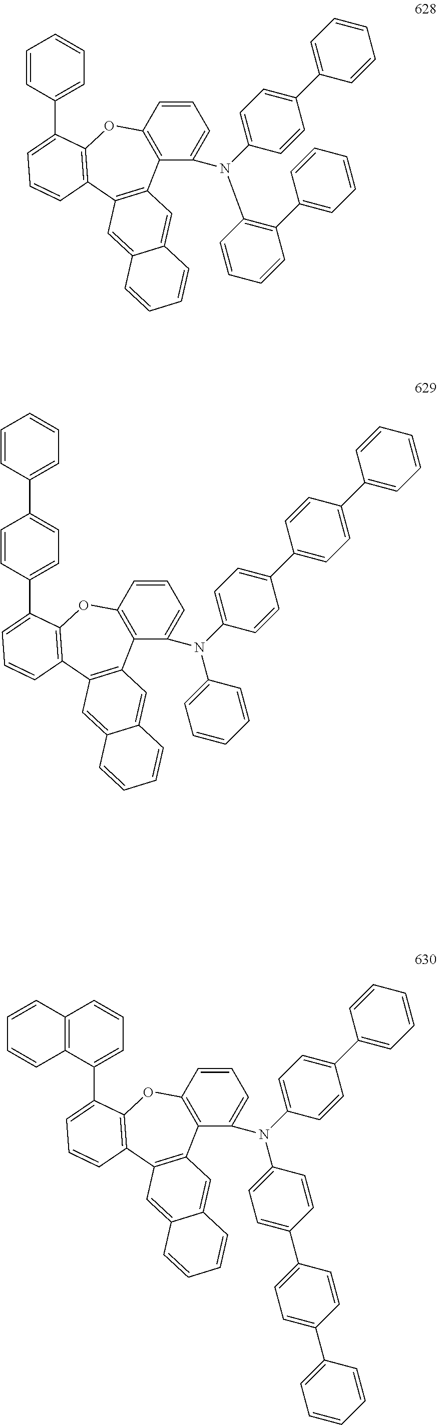 [SMILES: c1ccc(-c2ccc(N(c3ccccc3-c3ccccc3)c3cccc4c3-c3cc5ccccc5cc3-c3cccc(-c5ccccc5)c3O4)cc2)cc1 ; c1ccc(-c2ccc(-c3ccc(N(c4ccccc4)c4cccc5c4-c4cc6ccccc6cc4-c4cccc(-c6ccc(-c7ccccc7)cc6)c4O5)cc3)cc2)cc1 ; c1ccc(-c2ccc(-c3ccc(N(c4ccc(-c5ccccc5)cc4)c4cccc5c4-c4cc6ccccc6cc4-c4cccc(-c6cccc7ccccc67)c4O5)cc3)cc2)cc1]

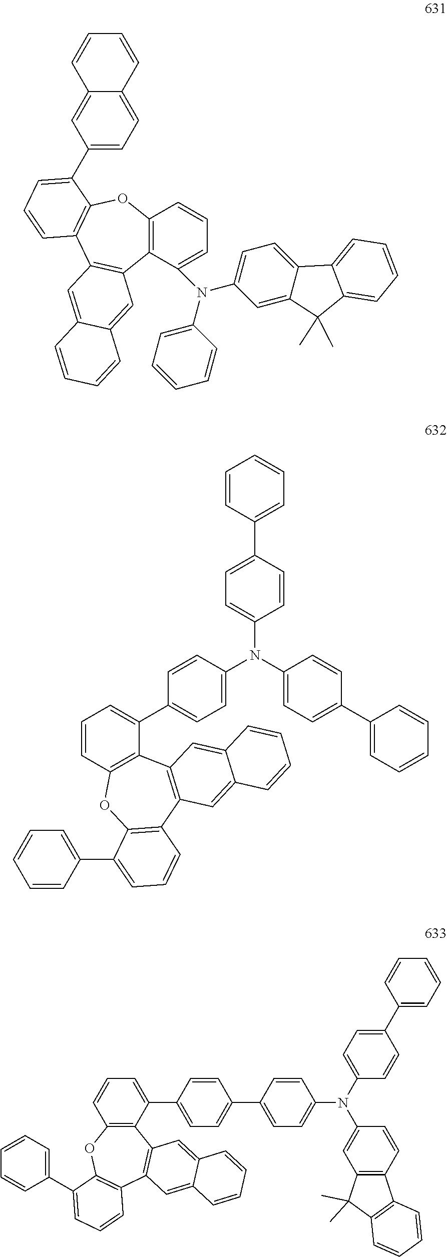 [SMILES: CC1(C)c2ccccc2-c2ccc(N(c3ccccc3)c3cccc4c3-c3cc5ccccc5cc3-c3cccc(-c5ccc6ccccc6c5)c3O4)cc21 ; c1ccc(-c2ccc(N(c3ccc(-c4ccccc4)cc3)c3ccc(-c4cccc5c4-c4cc6ccccc6cc4-c4cccc(-c6ccccc6)c4O5)cc3)cc2)cc1 ; CC1(C)c2ccccc2-c2ccc(N(c3ccc(-c4ccccc4)cc3)c3ccc(-c4ccc(-c5cccc6c5-c5cc7ccccc7cc5-c5cccc(-c7ccccc7)c5O6)cc4)cc3)cc21]

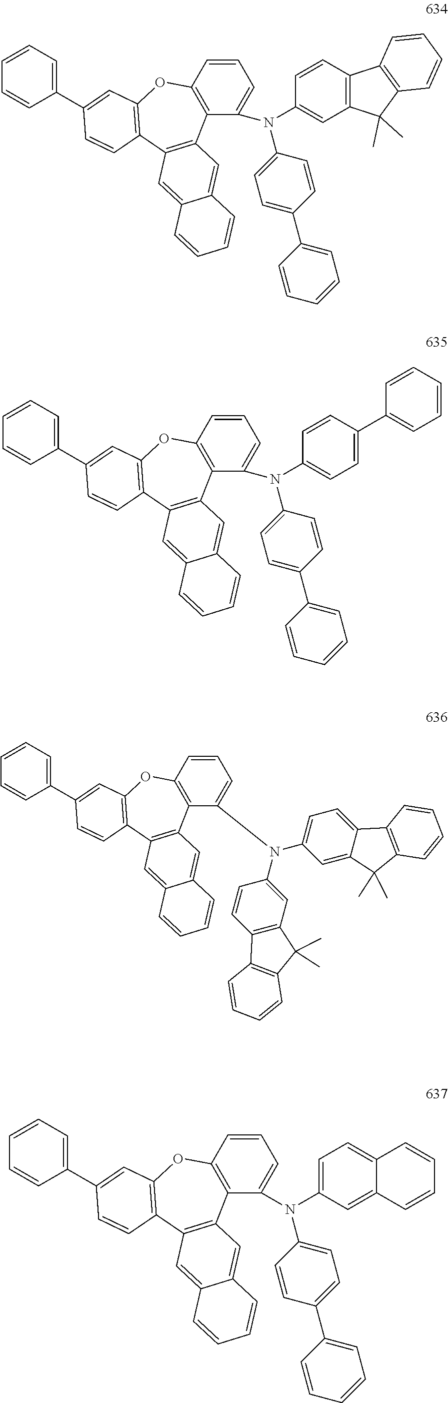 [SMILES: CC1(C)c2ccccc2-c2ccc(N(c3ccc(-c4ccccc4)cc3)c3cccc4c3-c3cc5ccccc5cc3-c3ccc(-c5ccccc5)cc3O4)cc21 ; c1ccc(-c2ccc(N(c3ccc(-c4ccccc4)cc3)c3cccc4c3-c3cc5ccccc5cc3-c3ccc(-c5ccccc5)cc3O4)cc2)cc1 ; CC1(C)c2ccccc2-c2ccc(N(c3ccc4c(c3)C(C)(C)c3ccccc3-4)c3cccc4c3-c3cc5ccccc5cc3-c3ccc(-c5ccccc5)cc3O4)cc21 ; c1ccc(-c2ccc(N(c3ccc4ccccc4c3)c3cccc4c3-c3cc5ccccc5cc3-c3ccc(-c5ccccc5)cc3O4)cc2)cc1]

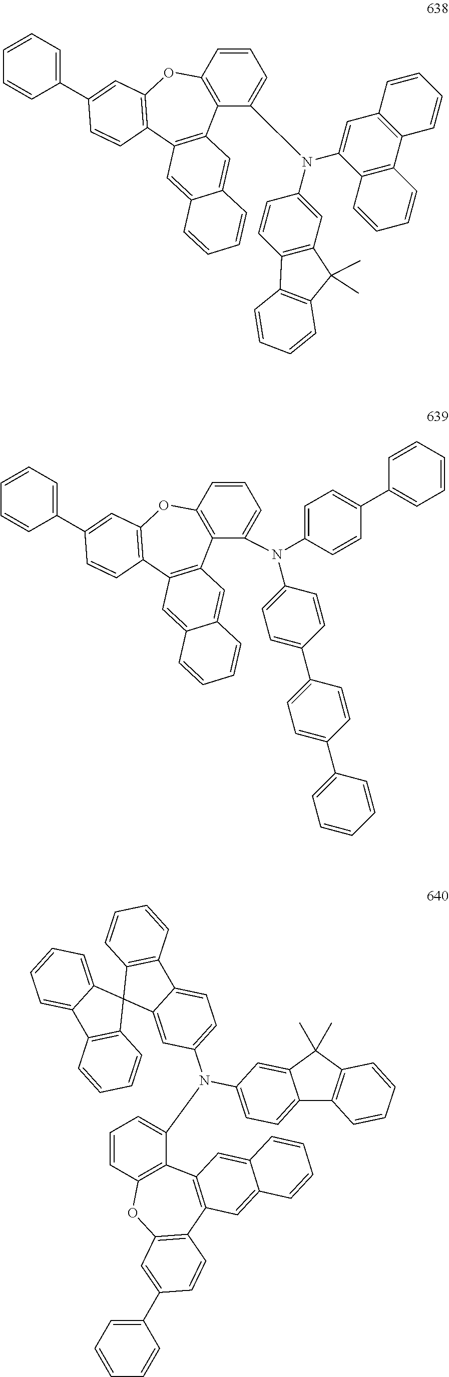 [SMILES: CC1(C)c2ccccc2-c2ccc(N(c3cccc4c3-c3cc5ccccc5cc3-c3ccc(-c5ccccc5)cc3O4)c3cc4ccccc4c4ccccc34)cc21 ; c1ccc(-c2ccc(-c3ccc(N(c4ccc(-c5ccccc5)cc4)c4cccc5c4-c4cc6ccccc6cc4-c4ccc(-c6ccccc6)cc4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3cc5ccccc5cc3-c3ccc(-c5ccccc5)cc3O4)cc21]

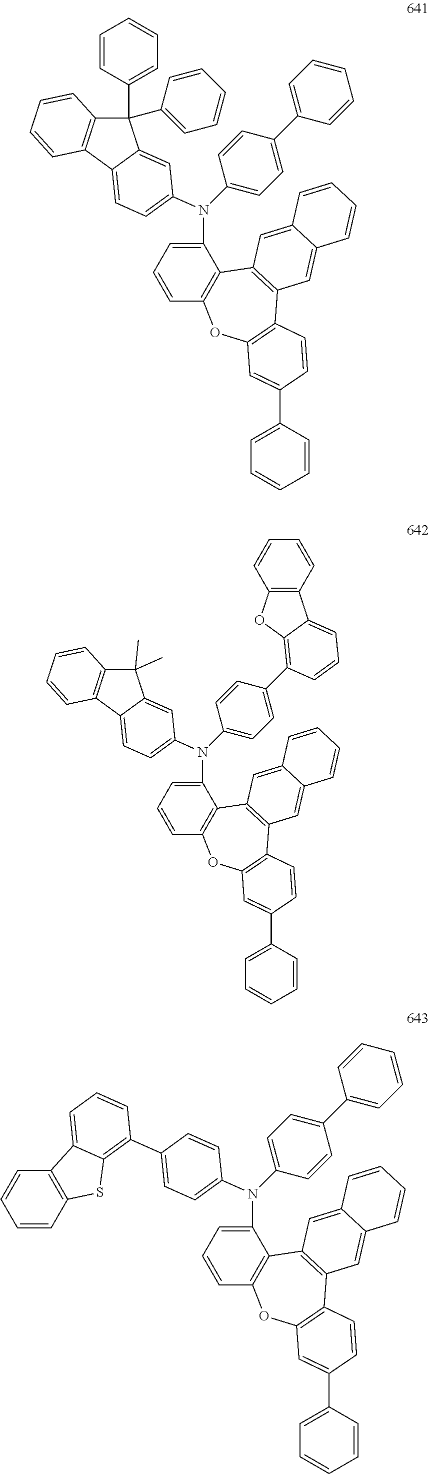 [SMILES: c1ccc(-c2ccc(N(c3ccc4c(c3)C(c3ccccc3)(c3ccccc3)c3ccccc3-4)c3cccc4c3-c3cc5ccccc5cc3-c3ccc(-c5ccccc5)cc3O4)cc2)cc1 ; CC1(C)c2ccccc2-c2ccc(N(c3ccc(-c4cccc5c4oc4ccccc45)cc3)c3cccc4c3-c3cc5ccccc5cc3-c3ccc(-c5ccccc5)cc3O4)cc21 ; c1ccc(-c2ccc(N(c3ccc(-c4cccc5c4sc4ccccc45)cc3)c3cccc4c3-c3cc5ccccc5cc3-c3ccc(-c5ccccc5)cc3O4)cc2)cc1]

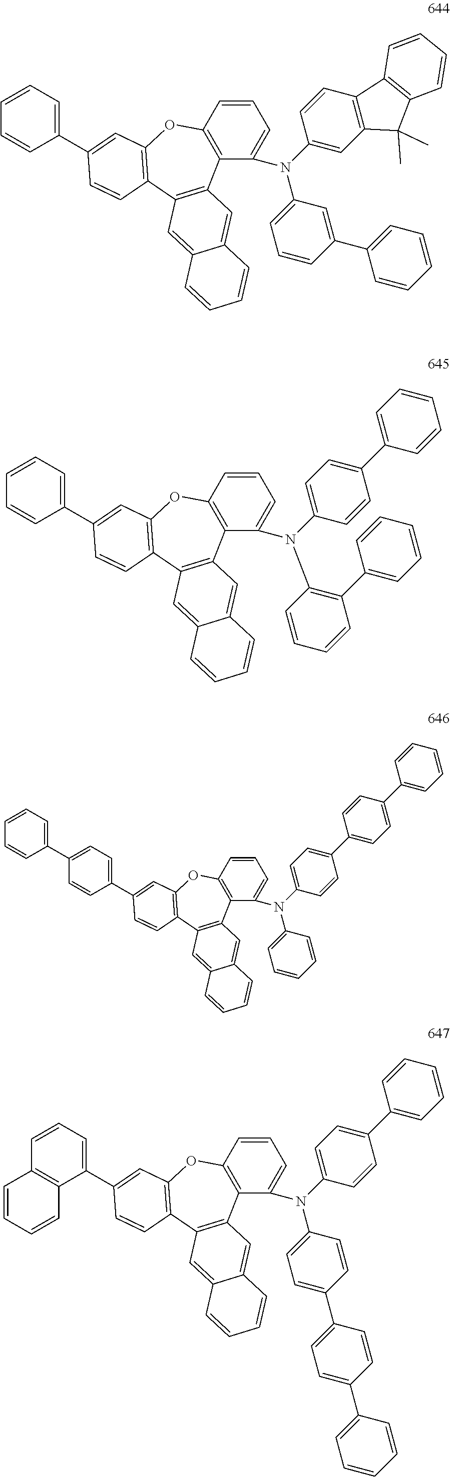 [SMILES: CC1(C)c2ccccc2-c2ccc(N(c3cccc(-c4ccccc4)c3)c3cccc4c3-c3cc5ccccc5cc3-c3ccc(-c5ccccc5)cc3O4)cc21 ; c1ccc(-c2ccc(N(c3ccccc3-c3ccccc3)c3cccc4c3-c3cc5ccccc5cc3-c3ccc(-c5ccccc5)cc3O4)cc2)cc1 ; c1ccc(-c2ccc(-c3ccc(N(c4ccccc4)c4cccc5c4-c4cc6ccccc6cc4-c4ccc(-c6ccc(-c7ccccc7)cc6)cc4O5)cc3)cc2)cc1 ; c1ccc(-c2ccc(-c3ccc(N(c4ccc(-c5ccccc5)cc4)c4cccc5c4-c4cc6ccccc6cc4-c4ccc(-c6cccc7ccccc67)cc4O5)cc3)cc2)cc1]

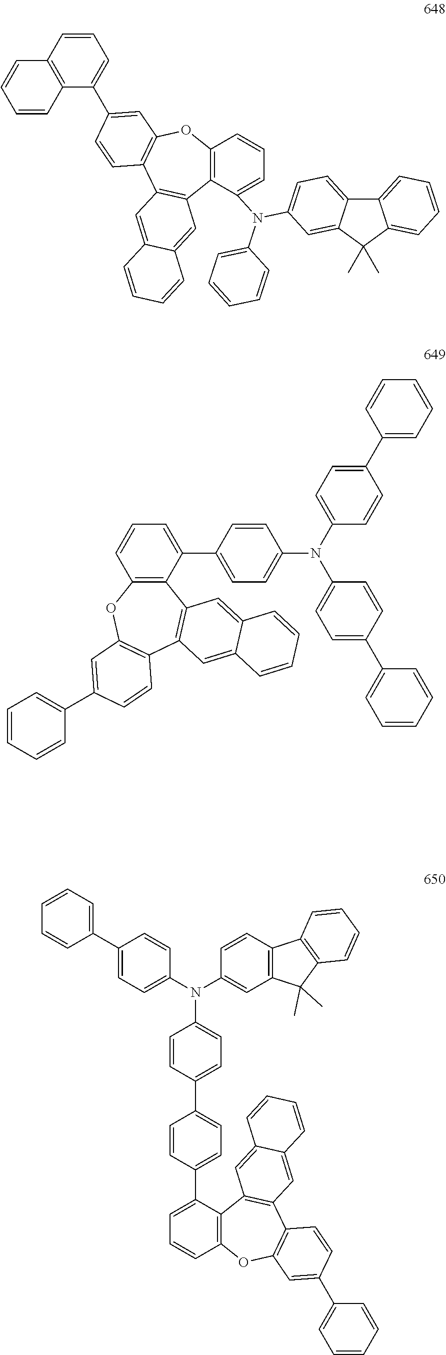 [SMILES: CC1(C)c2ccccc2-c2ccc(N(c3ccccc3)c3cccc4c3-c3cc5ccccc5cc3-c3ccc(-c5cccc6ccccc56)cc3O4)cc21 ; c1ccc(-c2ccc(N(c3ccc(-c4ccccc4)cc3)c3ccc(-c4cccc5c4-c4cc6ccccc6cc4-c4ccc(-c6ccccc6)cc4O5)cc3)cc2)cc1 ; CC1(C)c2ccccc2-c2ccc(N(c3ccc(-c4ccccc4)cc3)c3ccc(-c4ccc(-c5cccc6c5-c5cc7ccccc7cc5-c5ccc(-c7ccccc7)cc5O6)cc4)cc3)cc21]

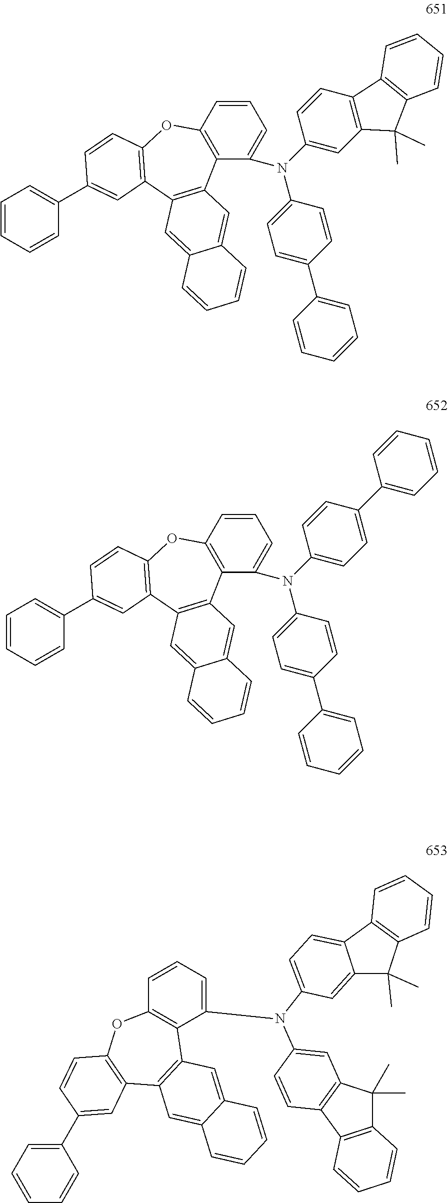 [SMILES: CC1(C)c2ccccc2-c2ccc(N(c3ccc(-c4ccccc4)cc3)c3cccc4c3-c3cc5ccccc5cc3-c3cc(-c5ccccc5)ccc3O4)cc21 ; c1ccc(-c2ccc(N(c3ccc(-c4ccccc4)cc3)c3cccc4c3-c3cc5ccccc5cc3-c3cc(-c5ccccc5)ccc3O4)cc2)cc1 ; CC1(C)c2ccccc2-c2ccc(N(c3ccc4c(c3)C(C)(C)c3ccccc3-4)c3cccc4c3-c3cc5ccccc5cc3-c3cc(-c5ccccc5)ccc3O4)cc21]

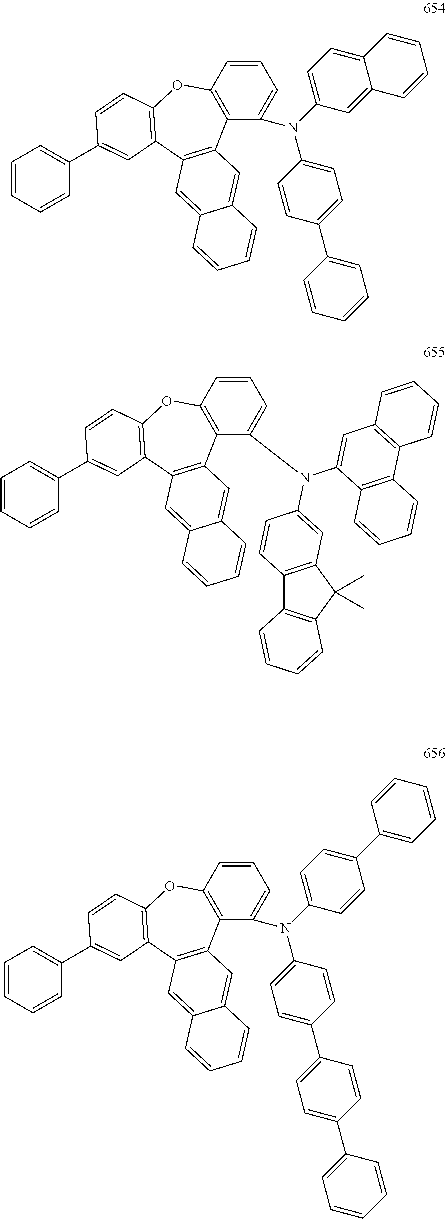 [SMILES: c1ccc(-c2ccc(N(c3ccc4ccccc4c3)c3cccc4c3-c3cc5ccccc5cc3-c3cc(-c5ccccc5)ccc3O4)cc2)cc1 ; CC1(C)c2ccccc2-c2ccc(N(c3cccc4c3-c3cc5ccccc5cc3-c3cc(-c5ccccc5)ccc3O4)c3cc4ccccc4c4ccccc34)cc21 ; c1ccc(-c2ccc(-c3ccc(N(c4ccc(-c5ccccc5)cc4)c4cccc5c4-c4cc6ccccc6cc4-c4cc(-c6ccccc6)ccc4O5)cc3)cc2)cc1]

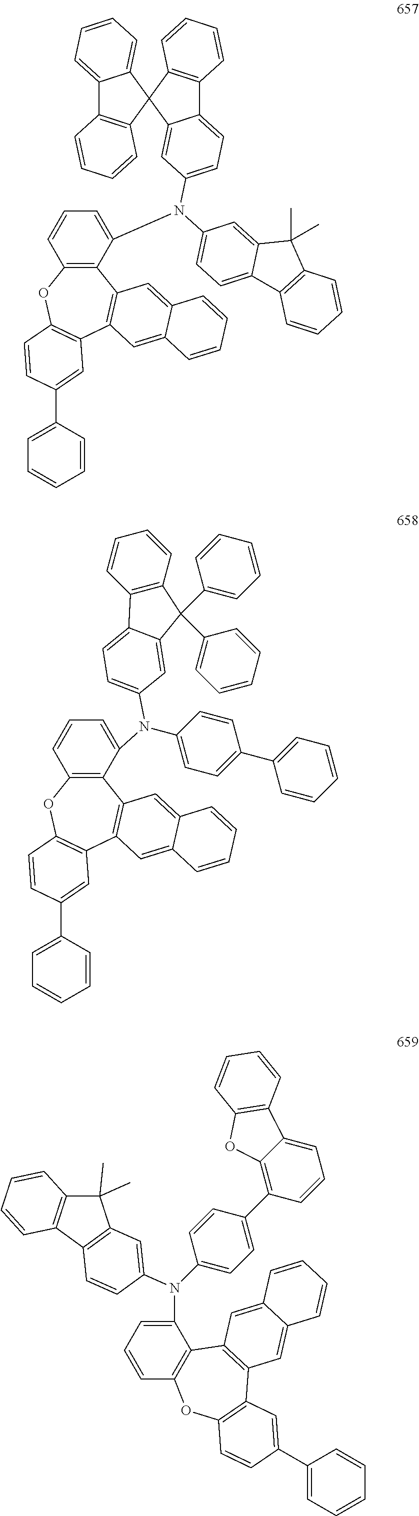 [SMILES: CC1(C)c2ccccc2-c2ccc(N(c3ccc4c(c3)C3(c5ccccc5-c5ccccc53)c3ccccc3-4)c3cccc4c3-c3cc5ccccc5cc3-c3cc(-c5ccccc5)ccc3O4)cc21 ; c1ccc(-c2ccc(N(c3ccc4c(c3)C(c3ccccc3)(c3ccccc3)c3ccccc3-4)c3cccc4c3-c3cc5ccccc5cc3-c3cc(-c5ccccc5)ccc3O4)cc2)cc1 ; CC1(C)c2ccccc2-c2ccc(N(c3ccc(-c4cccc5c4oc4ccccc45)cc3)c3cccc4c3-c3cc5ccccc5cc3-c3cc(-c5ccccc5)ccc3O4)cc21]

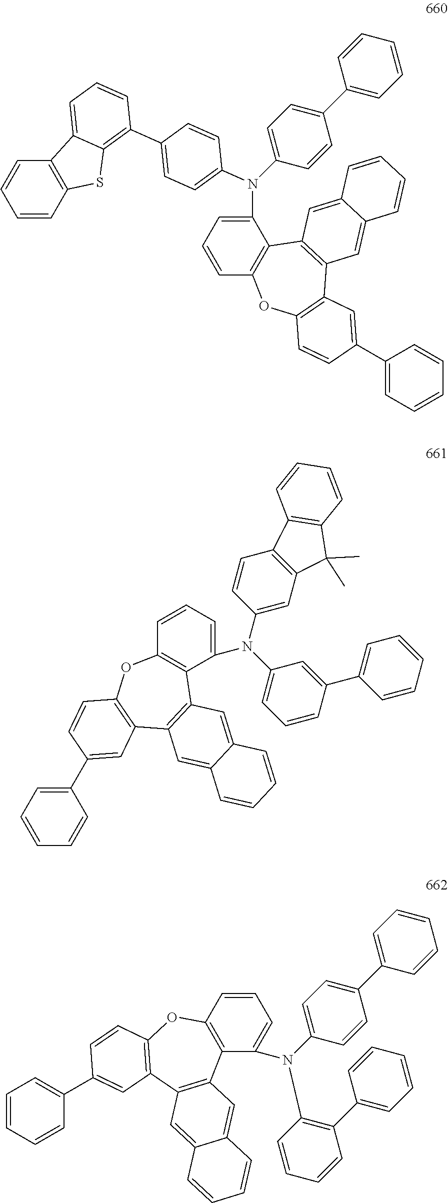 [SMILES: c1ccc(-c2ccc(N(c3ccc(-c4cccc5c4sc4ccccc45)cc3)c3cccc4c3-c3cc5ccccc5cc3-c3cc(-c5ccccc5)ccc3O4)cc2)cc1 ; CC1(C)c2ccccc2-c2ccc(N(c3cccc(-c4ccccc4)c3)c3cccc4c3-c3cc5ccccc5cc3-c3cc(-c5ccccc5)ccc3O4)cc21 ; c1ccc(-c2ccc(N(c3ccccc3-c3ccccc3)c3cccc4c3-c3cc5ccccc5cc3-c3cc(-c5ccccc5)ccc3O4)cc2)cc1]

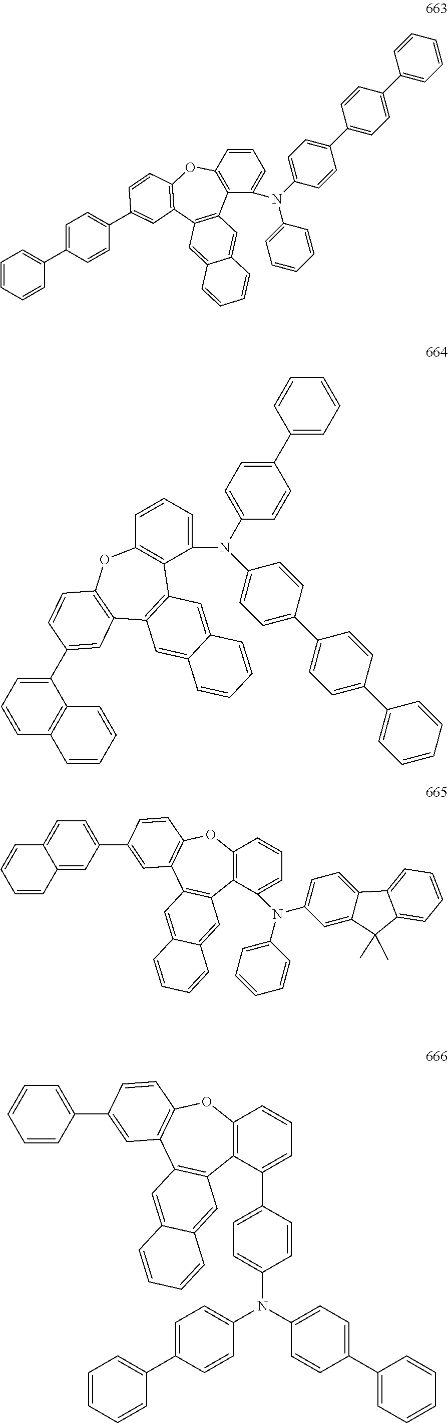 [SMILES: c1ccc(-c2ccc(-c3ccc(N(c4ccccc4)c4cccc5c4-c4cc6ccccc6cc4-c4cc(-c6ccc(-c7ccccc7)cc6)ccc4O5)cc3)cc2)cc1 ; c1ccc(-c2ccc(-c3ccc(N(c4ccc(-c5ccccc5)cc4)c4cccc5c4-c4cc6ccccc6cc4-c4cc(-c6cccc7ccccc67)ccc4O5)cc3)cc2)cc1 ; CC1(C)c2ccccc2-c2ccc(N(c3ccccc3)c3cccc4c3-c3cc5ccccc5cc3-c3cc(-c5ccc6ccccc6c5)ccc3O4)cc21 ; c1ccc(-c2ccc(N(c3ccc(-c4ccccc4)cc3)c3ccc(-c4cccc5c4-c4cc6ccccc6cc4-c4cc(-c6ccccc6)ccc4O5)cc3)cc2)cc1]

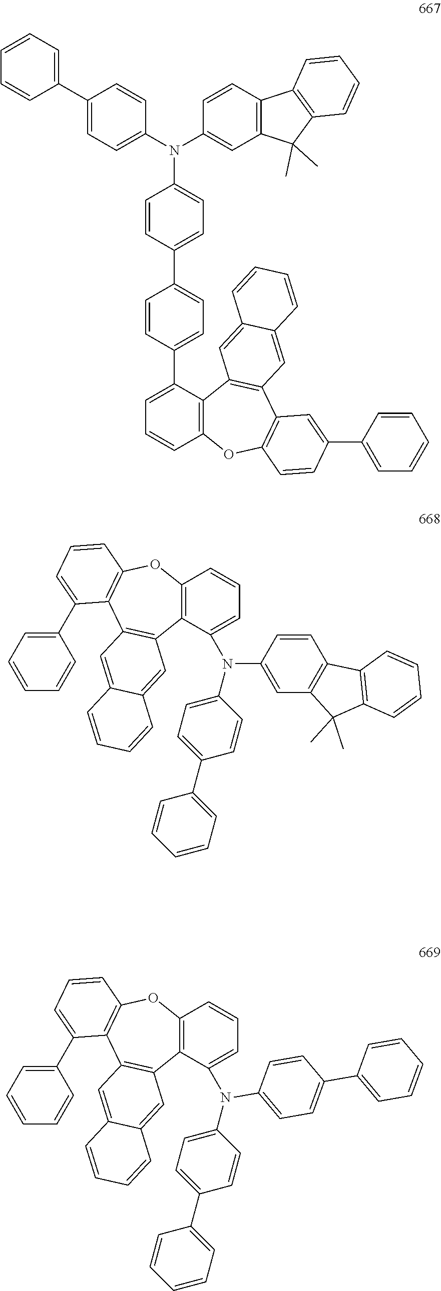 [SMILES: CC1(C)c2ccccc2-c2ccc(N(c3ccc(-c4ccccc4)cc3)c3ccc(-c4ccc(-c5cccc6c5-c5cc7ccccc7cc5-c5cc(-c7ccccc7)ccc5O6)cc4)cc3)cc21 ; CC1(C)c2ccccc2-c2ccc(N(c3ccc(-c4ccccc4)cc3)c3cccc4c3-c3cc5ccccc5cc3-c3c(cccc3-c3ccccc3)O4)cc21 ; c1ccc(-c2ccc(N(c3ccc(-c4ccccc4)cc3)c3cccc4c3-c3cc5ccccc5cc3-c3c(cccc3-c3ccccc3)O4)cc2)cc1]

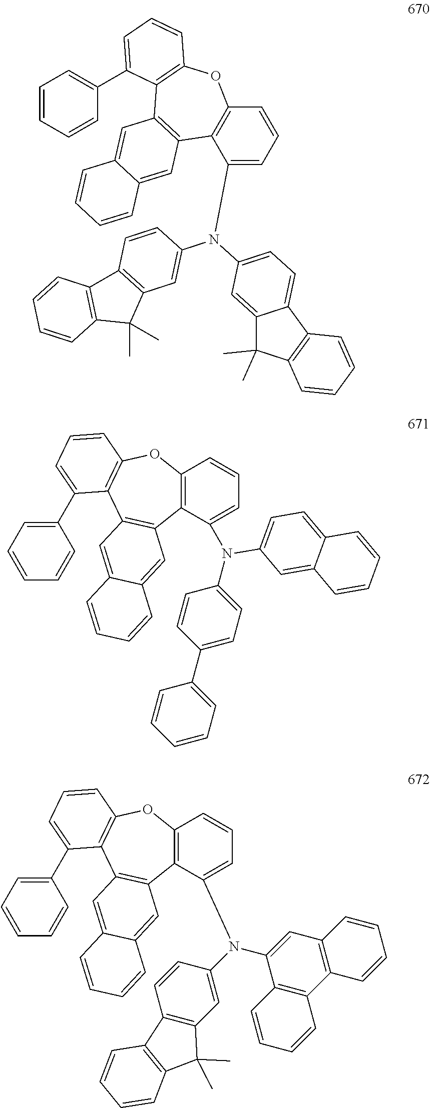 [SMILES: CC1(C)c2ccccc2-c2ccc(N(c3ccc4c(c3)C(C)(C)c3ccccc3-4)c3cccc4c3-c3cc5ccccc5cc3-c3c(cccc3-c3ccccc3)O4)cc21 ; c1ccc(-c2ccc(N(c3ccc4ccccc4c3)c3cccc4c3-c3cc5ccccc5cc3-c3c(cccc3-c3ccccc3)O4)cc2)cc1 ; CC1(C)c2ccccc2-c2ccc(N(c3cccc4c3-c3cc5ccccc5cc3-c3c(cccc3-c3ccccc3)O4)c3cc4ccccc4c4ccccc34)cc21]

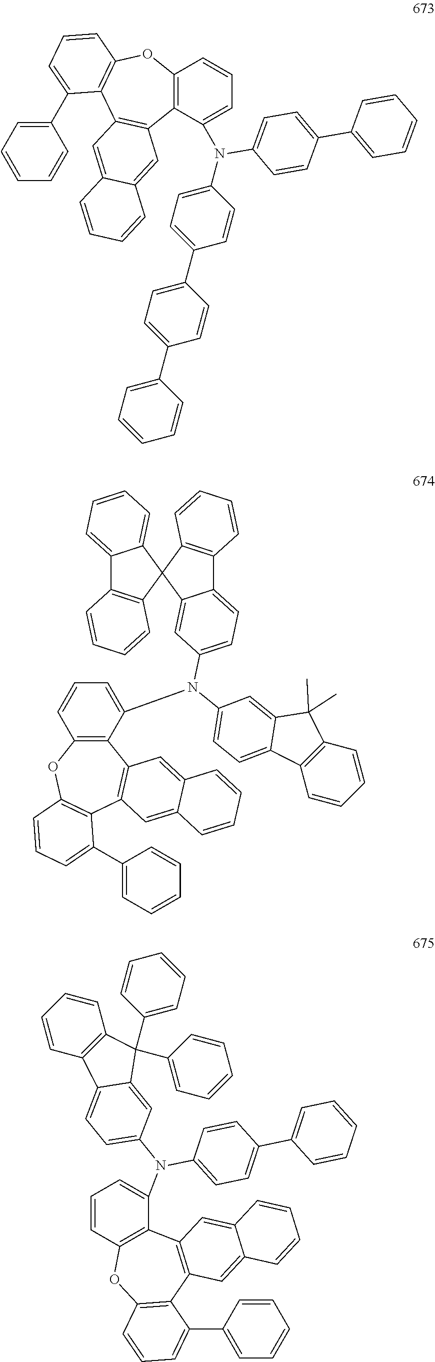

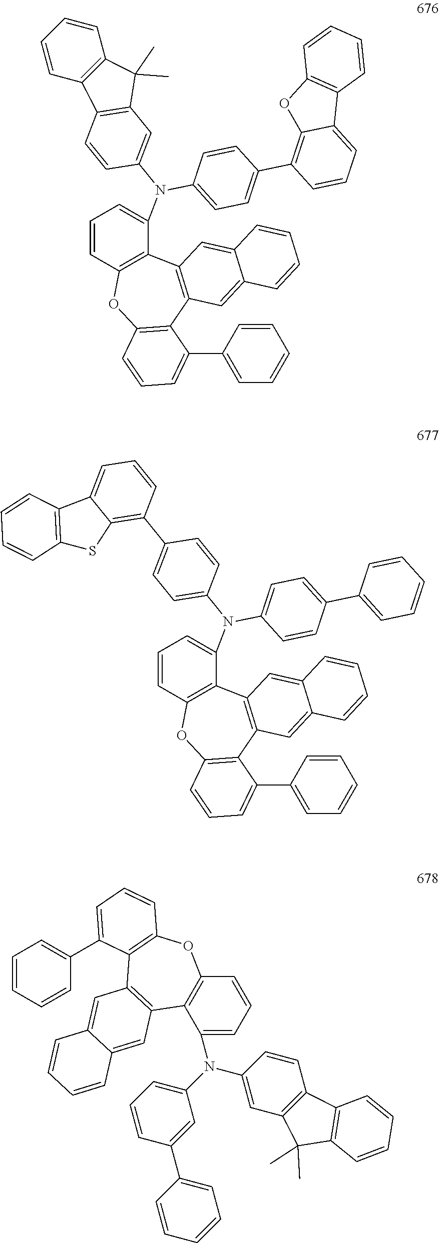 [SMILES: CC1(C)c2ccccc2-c2ccc(N(c3ccc(-c4cccc5c4oc4ccccc45)cc3)c3cccc4c3-c3cc5ccccc5cc3-c3c(cccc3-c3ccccc3)O4)cc21 ; c1ccc(-c2ccc(N(c3ccc(-c4cccc5c4sc4ccccc45)cc3)c3cccc4c3-c3cc5ccccc5cc3-c3c(cccc3-c3ccccc3)O4)cc2)cc1 ; CC1(C)c2ccccc2-c2ccc(N(c3cccc(-c4ccccc4)c3)c3cccc4c3-c3cc5ccccc5cc3-c3c(cccc3-c3ccccc3)O4)cc21]

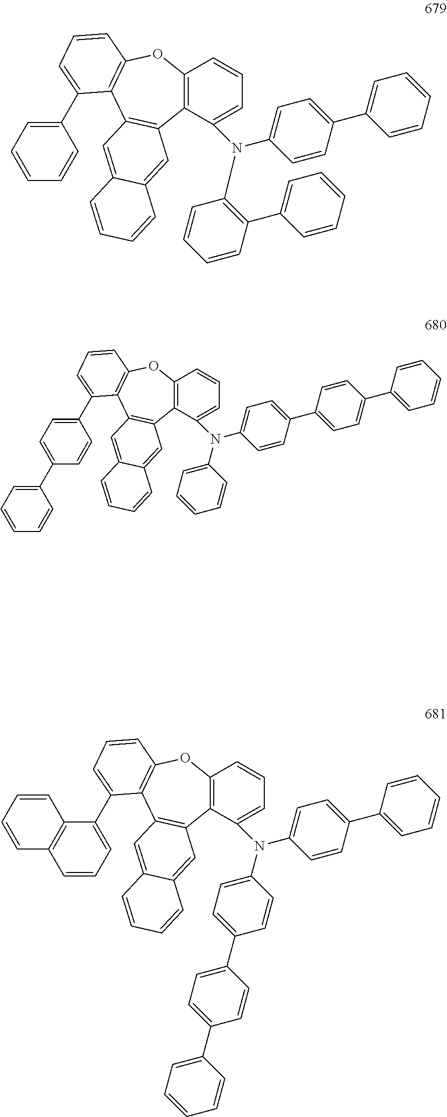 [SMILES: c1ccc(-c2ccc(N(c3ccccc3-c3ccccc3)c3cccc4c3-c3cc5ccccc5cc3-c3c(cccc3-c3ccccc3)O4)cc2)cc1 ; c1ccc(-c2ccc(-c3ccc(N(c4ccccc4)c4cccc5c4-c4cc6ccccc6cc4-c4c(cccc4-c4ccc(-c6ccccc6)cc4)O5)cc3)cc2)cc1 ; c1ccc(-c2ccc(-c3ccc(N(c4ccc(-c5ccccc5)cc4)c4cccc5c4-c4cc6ccccc6cc4-c4c(cccc4-c4cccc6ccccc46)O5)cc3)cc2)cc1]

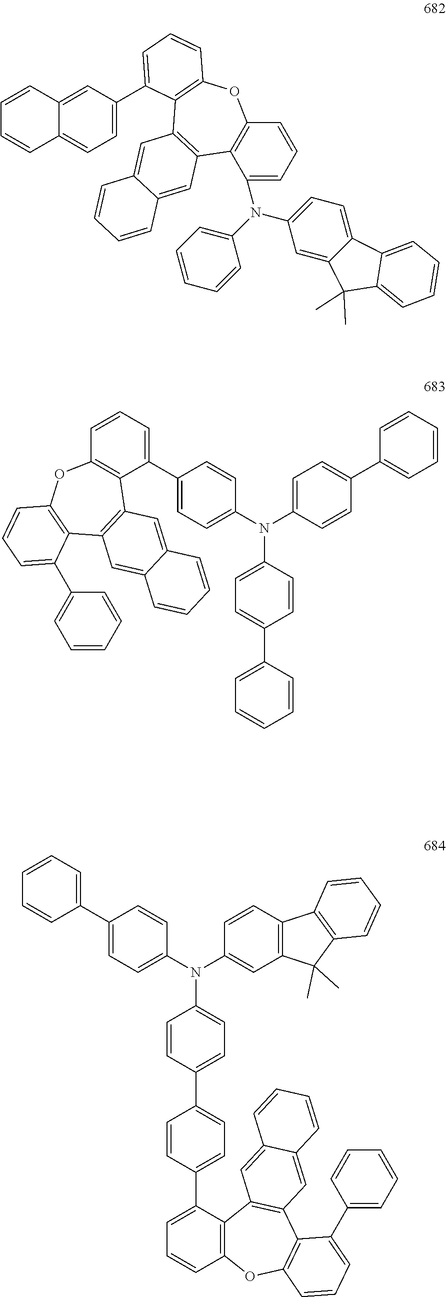 [SMILES: CC1(C)c2ccccc2-c2ccc(N(c3ccccc3)c3cccc4c3-c3cc5ccccc5cc3-c3c(cccc3-c3ccc5ccccc5c3)O4)cc21 ; c1ccc(-c2ccc(N(c3ccc(-c4ccccc4)cc3)c3ccc(-c4cccc5c4-c4cc6ccccc6cc4-c4c(cccc4-c4ccccc4)O5)cc3)cc2)cc1 ; CC1(C)c2ccccc2-c2ccc(N(c3ccc(-c4ccccc4)cc3)c3ccc(-c4ccc(-c5cccc6c5-c5cc7ccccc7cc5-c5c(cccc5-c5ccccc5)O6)cc4)cc3)cc21]

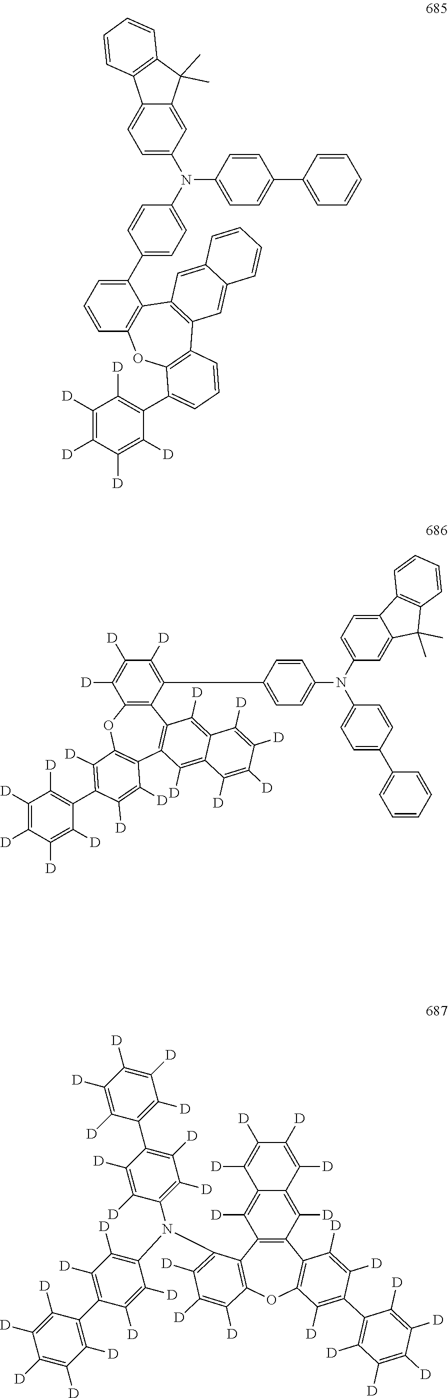

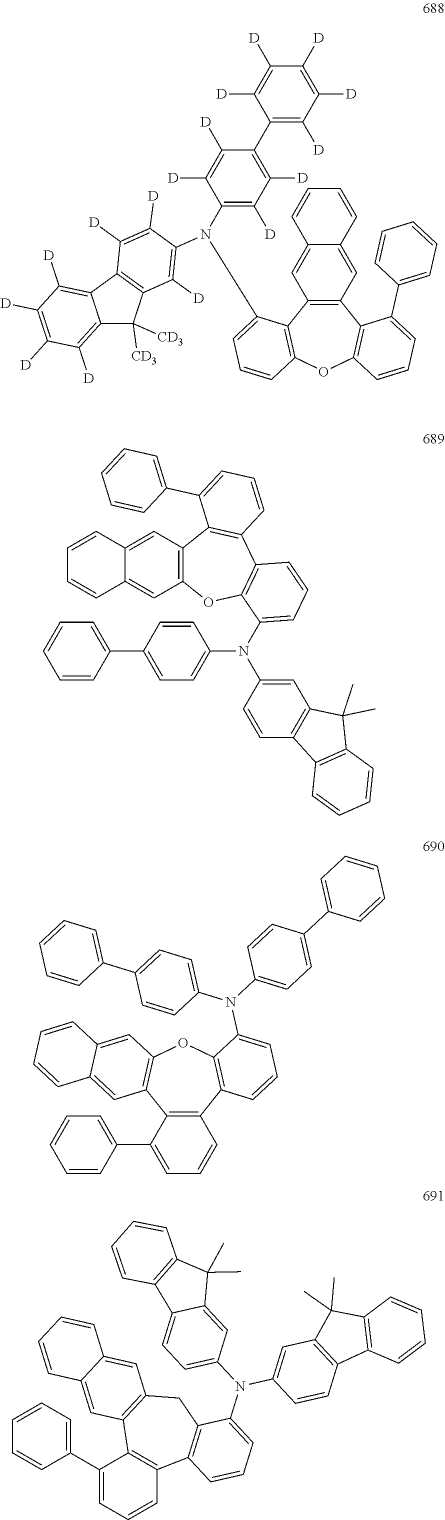

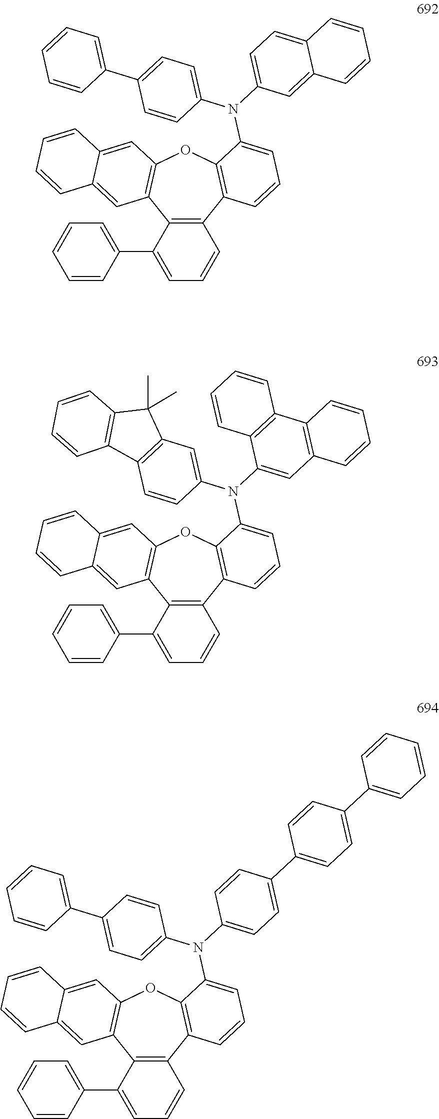 [SMILES: c1ccc(-c2ccc(N(c3ccc4ccccc4c3)c3cccc4c3Oc3cc5ccccc5cc3-c3c(-c5ccccc5)cccc3-4)cc2)cc1 ; CC1(C)c2ccccc2-c2ccc(N(c3cccc4c3Oc3cc5ccccc5cc3-c3c(-c5ccccc5)cccc3-4)c3cc4ccccc4c4ccccc34)cc21 ; c1ccc(-c2ccc(-c3ccc(N(c4ccc(-c5ccccc5)cc4)c4cccc5c4Oc4cc6ccccc6cc4-c4c(-c6ccccc6)cccc4-5)cc3)cc2)cc1]

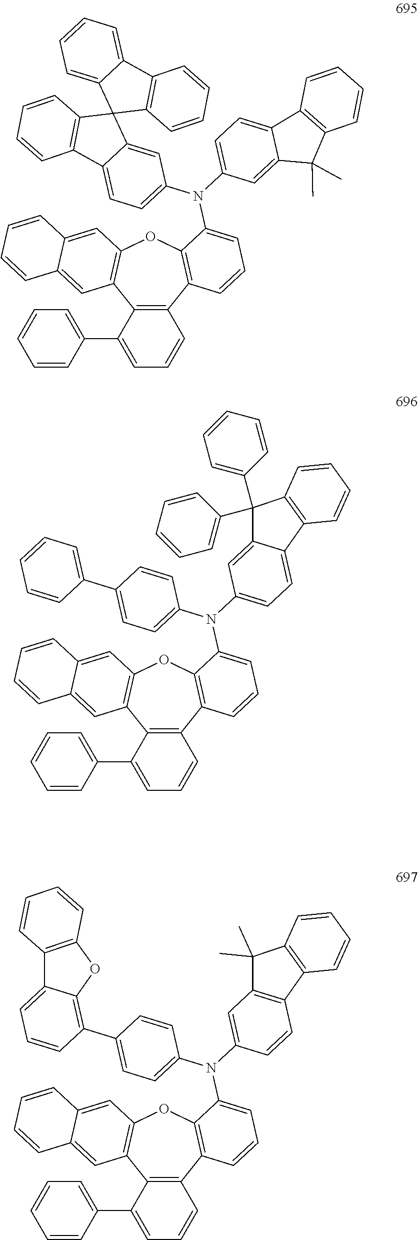 [SMILES: CC1(C)c2ccccc2-c2ccc(N(c3ccc4c(c3)C3(c5ccccc5-c5ccccc53)c3ccccc3-4)c3cccc4c3Oc3cc5ccccc5cc3-c3c(-c5ccccc5)cccc3-4)cc21 ; c1ccc(-c2ccc(N(c3ccc4c(c3)C(c3ccccc3)(c3ccccc3)c3ccccc3-4)c3cccc4c3Oc3cc5ccccc5cc3-c3c(-c5ccccc5)cccc3-4)cc2)cc1 ; CC1(C)c2ccccc2-c2ccc(N(c3ccc(-c4cccc5c4oc4ccccc45)cc3)c3cccc4c3Oc3cc5ccccc5cc3-c3c(-c5ccccc5)cccc3-4)cc21]

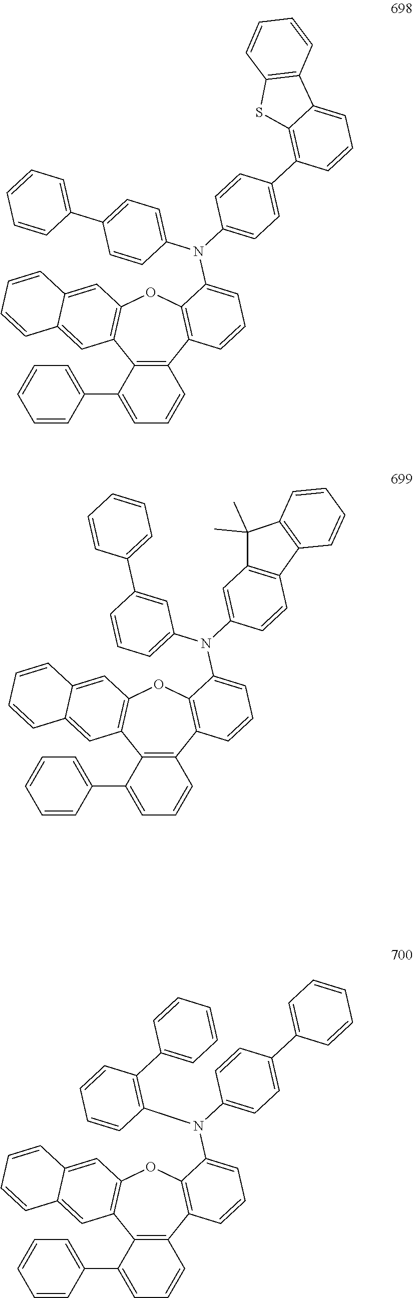 [SMILES: c1ccc(-c2ccc(N(c3ccc(-c4cccc5c4sc4ccccc45)cc3)c3cccc4c3Oc3cc5ccccc5cc3-c3c(-c5ccccc5)cccc3-4)cc2)cc1 ; CC1(C)c2ccccc2-c2ccc(N(c3cccc(-c4ccccc4)c3)c3cccc4c3Oc3cc5ccccc5cc3-c3c(-c5ccccc5)cccc3-4)cc21 ; c1ccc(-c2ccc(N(c3ccccc3-c3ccccc3)c3cccc4c3Oc3cc5ccccc5cc3-c3c(-c5ccccc5)cccc3-4)cc2)cc1]

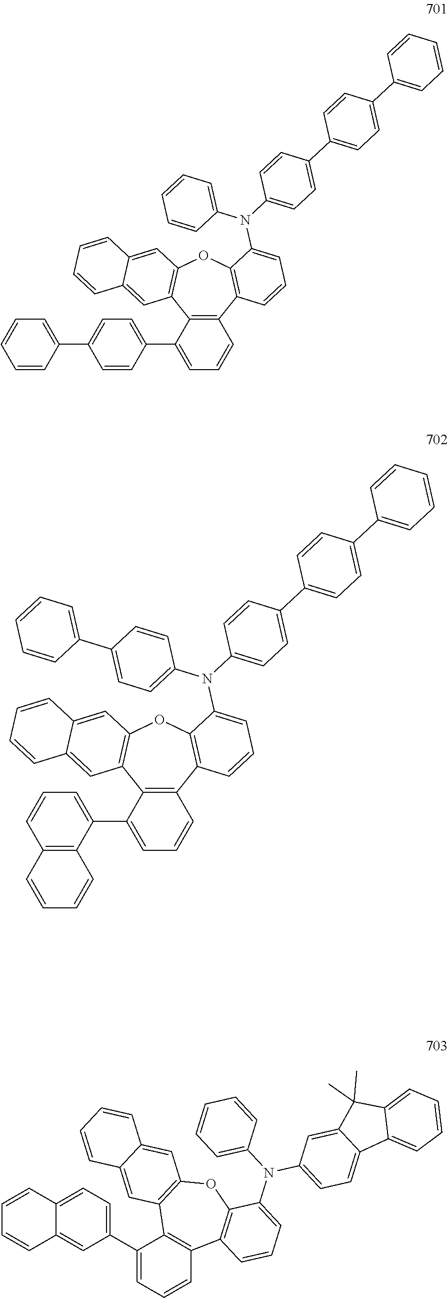 [SMILES: c1ccc(-c2ccc(-c3ccc(N(c4ccccc4)c4cccc5c4Oc4cc6ccccc6cc4-c4c(-c6ccc(-c7ccccc7)cc6)cccc4-5)cc3)cc2)cc1 ; c1ccc(-c2ccc(-c3ccc(N(c4ccc(-c5ccccc5)cc4)c4cccc5c4Oc4cc6ccccc6cc4-c4c-5cccc4-c4cccc5ccccc45)cc3)cc2)cc1 ; CC1(C)c2ccccc2-c2ccc(N(c3ccccc3)c3cccc4c3Oc3cc5ccccc5cc3-c3c(-c5ccc6ccccc6c5)cccc3-4)cc21]

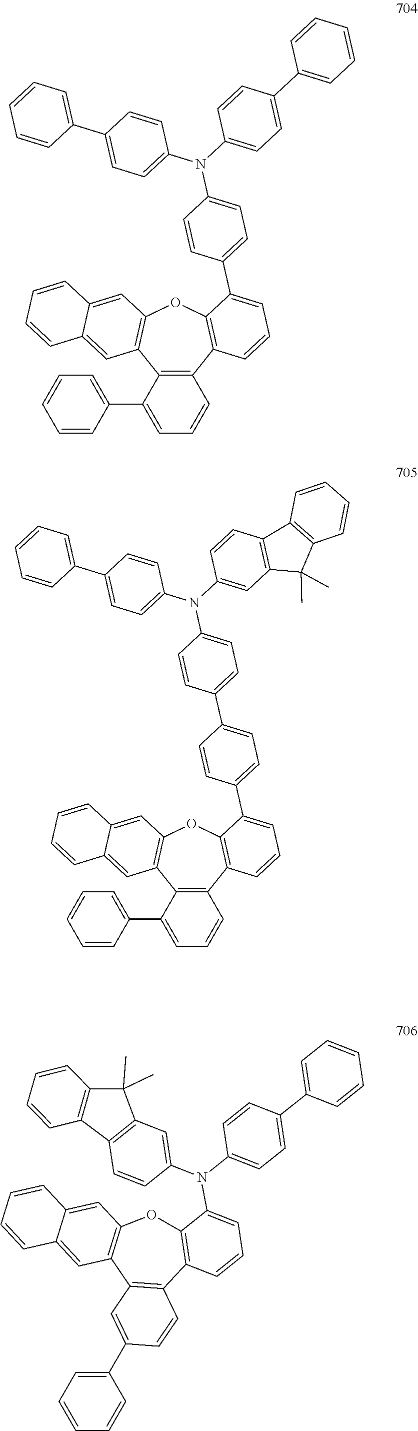 [SMILES: c1ccc(-c2ccc(N(c3ccc(-c4ccccc4)cc3)c3ccc(-c4cccc5c4Oc4cc6ccccc6cc4-c4c(-c6ccccc6)cccc4-5)cc3)cc2)cc1 ; CC1(C)c2ccccc2-c2ccc(N(c3ccc(-c4ccccc4)cc3)c3ccc(-c4ccc(-c5cccc6c5Oc5cc7ccccc7cc5-c5c(-c7ccccc7)cccc5-6)cc4)cc3)cc21 ; CC1(C)c2ccccc2-c2ccc(N(c3ccc(-c4ccccc4)cc3)c3cccc4c3Oc3cc5ccccc5cc3-c3cc(-c5ccccc5)ccc3-4)cc21]

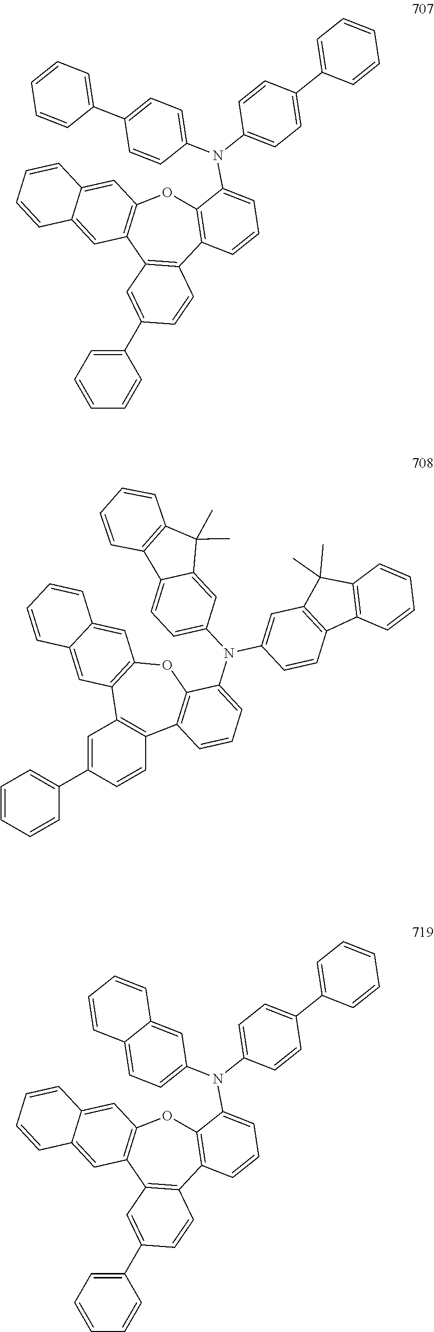 [SMILES: c1ccc(-c2ccc(N(c3ccc(-c4ccccc4)cc3)c3cccc4c3Oc3cc5ccccc5cc3-c3cc(-c5ccccc5)ccc3-4)cc2)cc1 ; CC1(C)c2ccccc2-c2ccc(N(c3ccc4c(c3)C(C)(C)c3ccccc3-4)c3cccc4c3Oc3cc5ccccc5cc3-c3cc(-c5ccccc5)ccc3-4)cc21 ; c1ccc(-c2ccc(N(c3ccc4ccccc4c3)c3cccc4c3Oc3cc5ccccc5cc3-c3cc(-c5ccccc5)ccc3-4)cc2)cc1]

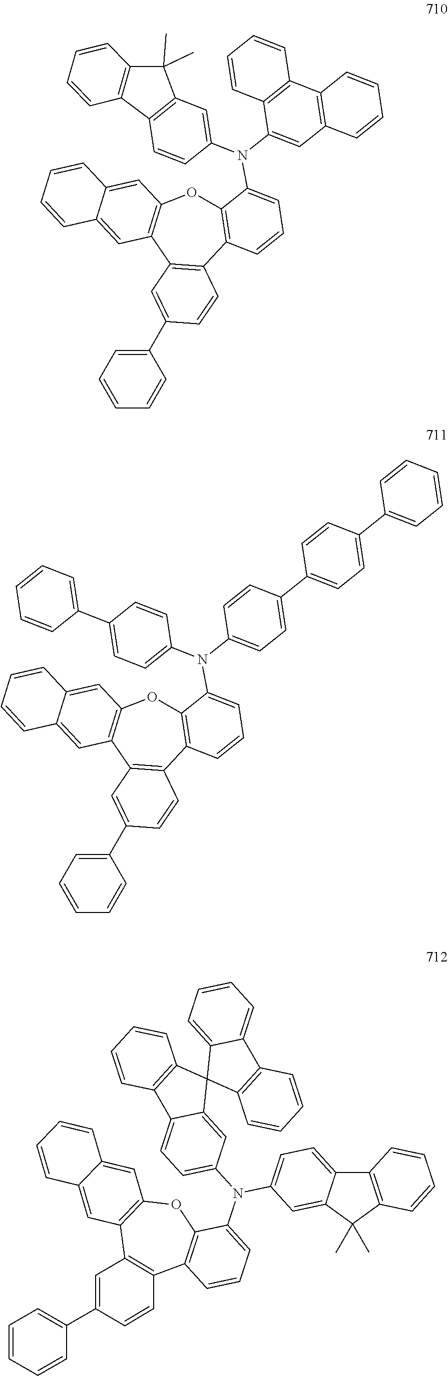 [SMILES: CC1(C)c2ccccc2-c2ccc(N(c3cccc4c3Oc3cc5ccccc5cc3-c3cc(-c5ccccc5)ccc3-4)c3cc4ccccc4c4ccccc34)cc21 ; c1ccc(-c2ccc(-c3ccc(N(c4ccc(-c5ccccc5)cc4)c4cccc5c4Oc4cc6ccccc6cc4-c4cc(-c6ccccc6)ccc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cc5ccccc5cc3-c3cc(-c5ccccc5)ccc3-4)cc21]

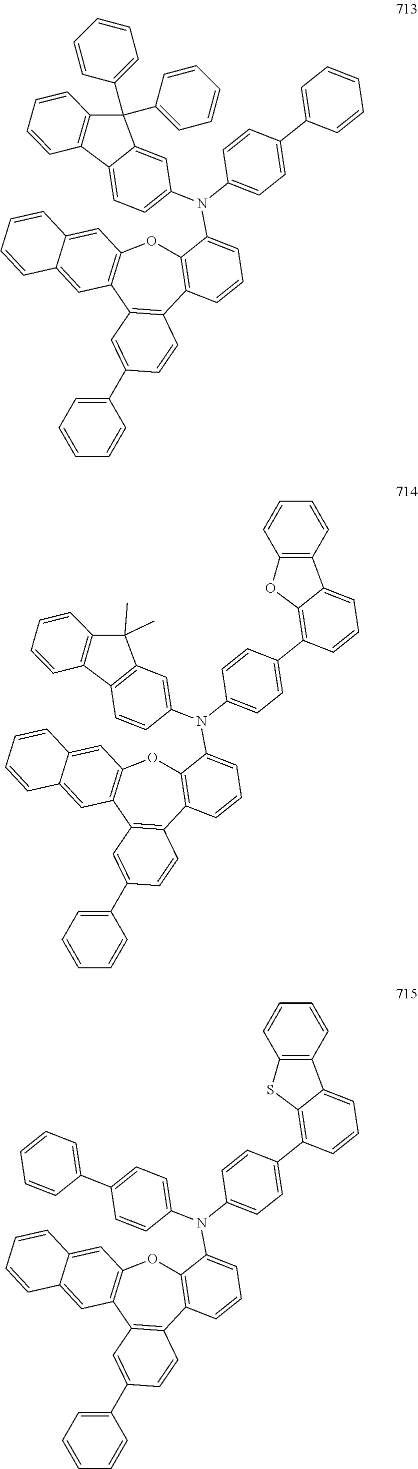 [SMILES: c1ccc(-c2ccc(N(c3ccc4c(c3)C(c3ccccc3)(c3ccccc3)c3ccccc3-4)c3cccc4c3Oc3cc5ccccc5cc3-c3cc(-c5ccccc5)ccc3-4)cc2)cc1 ; CC1(C)c2ccccc2-c2ccc(N(c3ccc(-c4cccc5c4oc4ccccc45)cc3)c3cccc4c3Oc3cc5ccccc5cc3-c3cc(-c5ccccc5)ccc3-4)cc21 ; c1ccc(-c2ccc(N(c3ccc(-c4cccc5c4sc4ccccc45)cc3)c3cccc4c3Oc3cc5ccccc5cc3-c3cc(-c5ccccc5)ccc3-4)cc2)cc1]

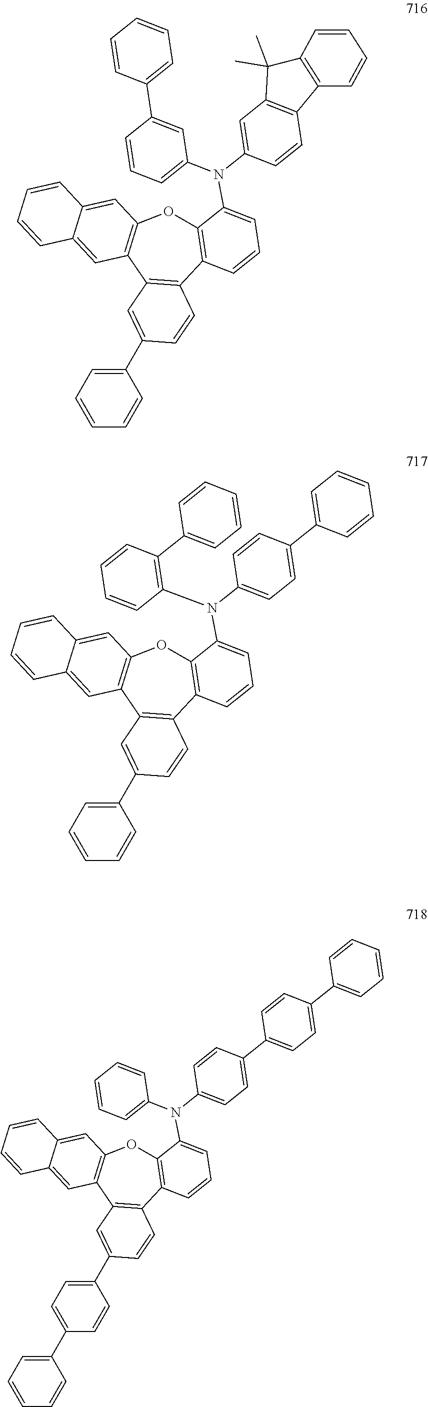 [SMILES: CC1(C)c2ccccc2-c2ccc(N(c3cccc(-c4ccccc4)c3)c3cccc4c3Oc3cc5ccccc5cc3-c3cc(-c5ccccc5)ccc3-4)cc21 ; c1ccc(-c2ccc(N(c3ccccc3-c3ccccc3)c3cccc4c3Oc3cc5ccccc5cc3-c3cc(-c5ccccc5)ccc3-4)cc2)cc1 ; c1ccc(-c2ccc(-c3ccc(N(c4ccccc4)c4cccc5c4Oc4cc6ccccc6cc4-c4cc(-c6ccc(-c7ccccc7)cc6)ccc4-5)cc3)cc2)cc1]

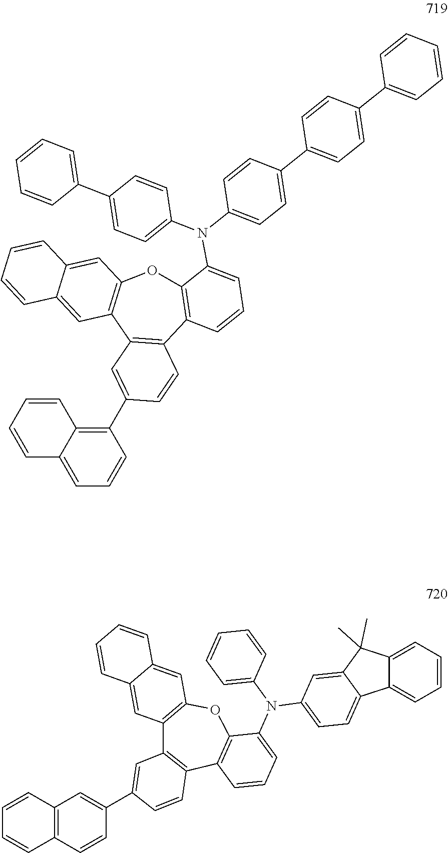 [SMILES: c1ccc(-c2ccc(-c3ccc(N(c4ccc(-c5ccccc5)cc4)c4cccc5c4Oc4cc6ccccc6cc4-c4cc(-c6cccc7ccccc67)ccc4-5)cc3)cc2)cc1 ; CC1(C)c2ccccc2-c2ccc(N(c3ccccc3)c3cccc4c3Oc3cc5ccccc5cc3-c3cc(-c5ccc6ccccc6c5)ccc3-4)cc21]

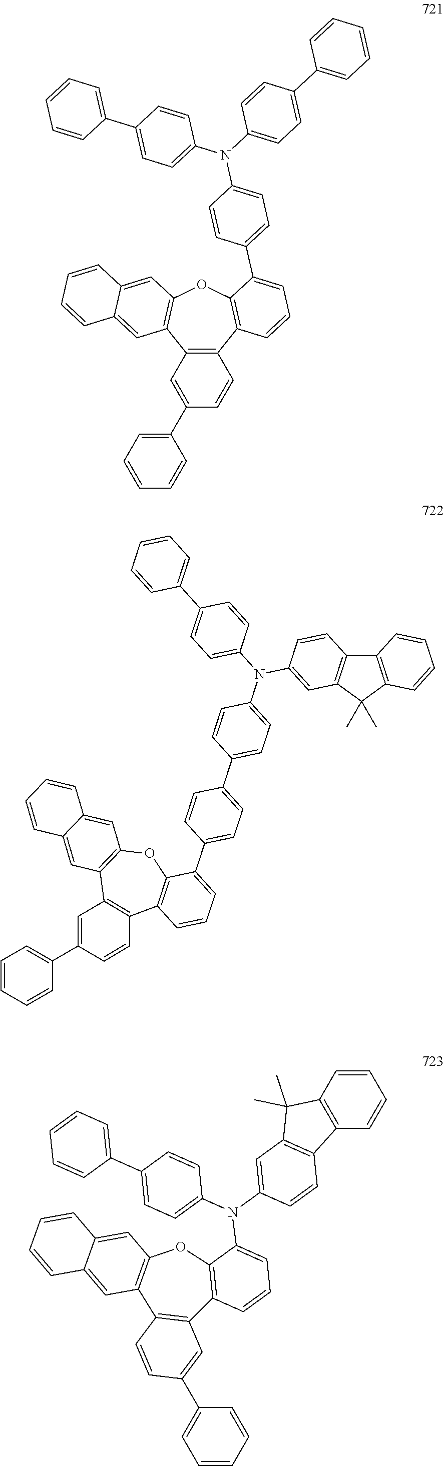 [SMILES: c1ccc(-c2ccc(N(c3ccc(-c4ccccc4)cc3)c3ccc(-c4cccc5c4Oc4cc6ccccc6cc4-c4cc(-c6ccccc6)ccc4-5)cc3)cc2)cc1 ; CC1(C)c2ccccc2-c2ccc(N(c3ccc(-c4ccccc4)cc3)c3ccc(-c4ccc(-c5cccc6c5Oc5cc7ccccc7cc5-c5cc(-c7ccccc7)ccc5-6)cc4)cc3)cc21 ; CC1(C)c2ccccc2-c2ccc(N(c3ccc(-c4ccccc4)cc3)c3cccc4c3Oc3cc5ccccc5cc3-c3ccc(-c5ccccc5)cc3-4)cc21]

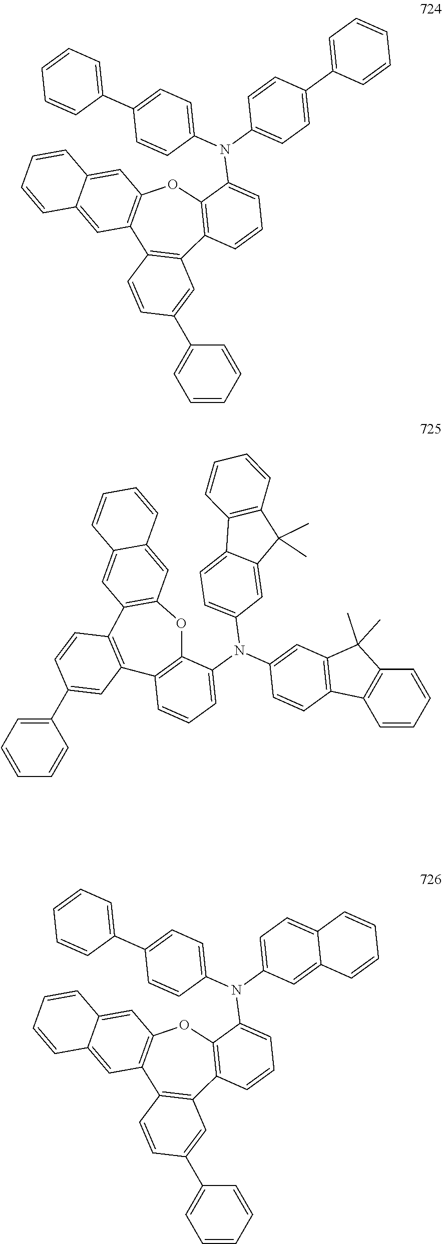 [SMILES: c1ccc(-c2ccc(N(c3ccc(-c4ccccc4)cc3)c3cccc4c3Oc3cc5ccccc5cc3-c3ccc(-c5ccccc5)cc3-4)cc2)cc1 ; CC1(C)c2ccccc2-c2ccc(N(c3ccc4c(c3)C(C)(C)c3ccccc3-4)c3cccc4c3Oc3cc5ccccc5cc3-c3ccc(-c5ccccc5)cc3-4)cc21 ; c1ccc(-c2ccc(N(c3ccc4ccccc4c3)c3cccc4c3Oc3cc5ccccc5cc3-c3ccc(-c5ccccc5)cc3-4)cc2)cc1]

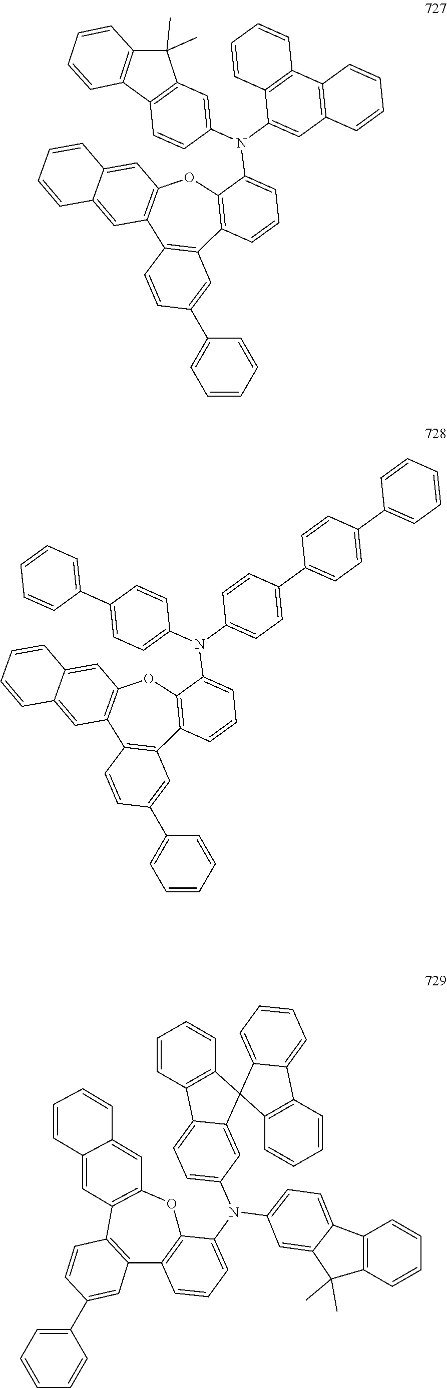 [SMILES: CC1(C)c2ccccc2-c2ccc(N(c3cccc4c3Oc3cc5ccccc5cc3-c3ccc(-c5ccccc5)cc3-4)c3cc4ccccc4c4ccccc34)cc21 ; c1ccc(-c2ccc(-c3ccc(N(c4ccc(-c5ccccc5)cc4)c4cccc5c4Oc4cc6ccccc6cc4-c4ccc(-c6ccccc6)cc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cc5ccccc5cc3-c3ccc(-c5ccccc5)cc3-4)cc21]

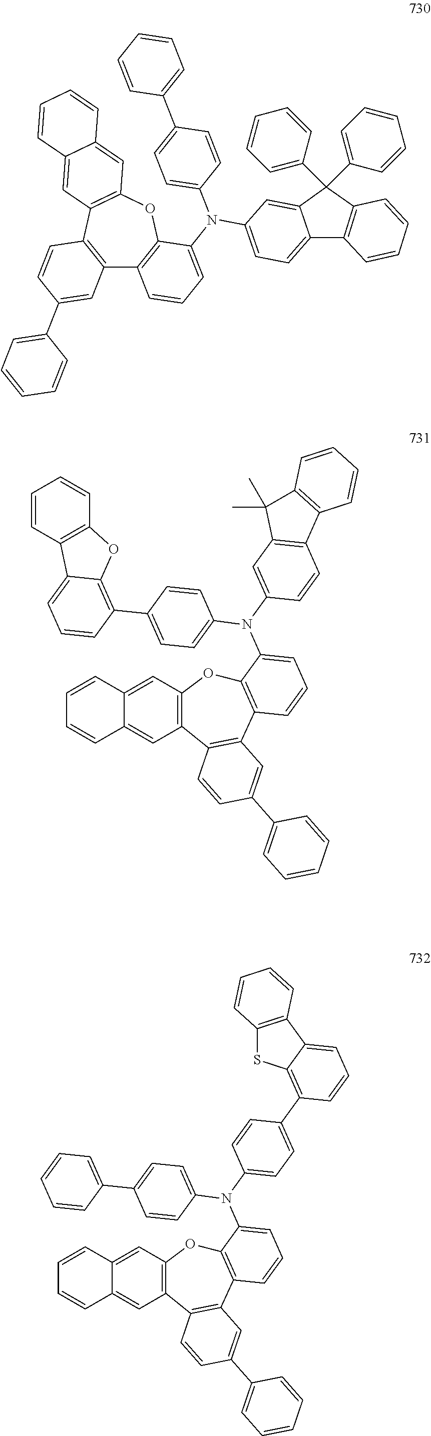 [SMILES: c1ccc(-c2ccc(N(c3ccc4c(c3)C(c3ccccc3)(c3ccccc3)c3ccccc3-4)c3cccc4c3Oc3cc5ccccc5cc3-c3ccc(-c5ccccc5)cc3-4)cc2)cc1 ; CC1(C)c2ccccc2-c2ccc(N(c3ccc(-c4cccc5c4oc4ccccc45)cc3)c3cccc4c3Oc3cc5ccccc5cc3-c3ccc(-c5ccccc5)cc3-4)cc21 ; c1ccc(-c2ccc(N(c3ccc(-c4cccc5c4sc4ccccc45)cc3)c3cccc4c3Oc3cc5ccccc5cc3-c3ccc(-c5ccccc5)cc3-4)cc2)cc1]

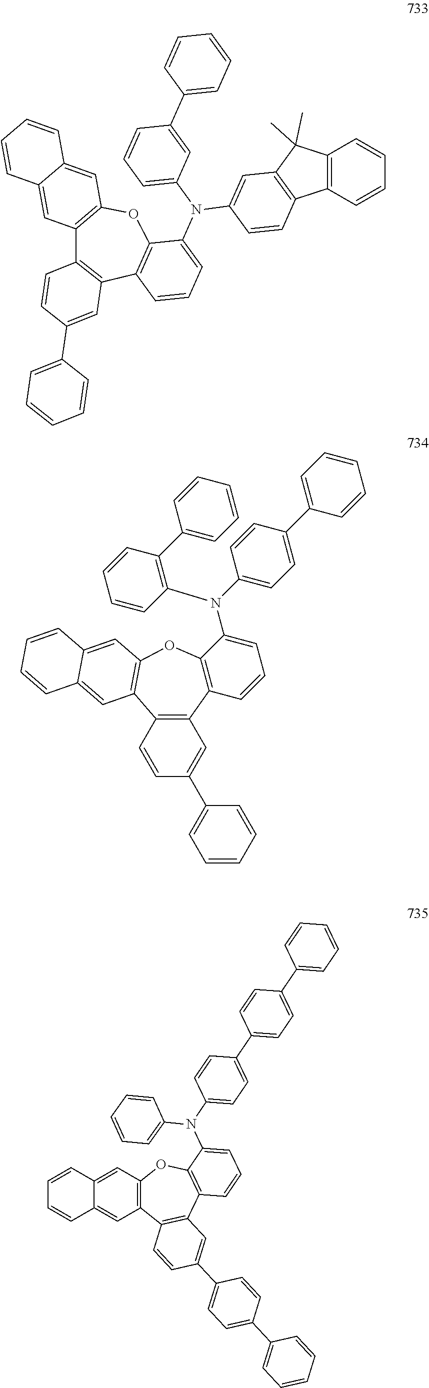 [SMILES: CC1(C)c2ccccc2-c2ccc(N(c3cccc(-c4ccccc4)c3)c3cccc4c3Oc3cc5ccccc5cc3-c3ccc(-c5ccccc5)cc3-4)cc21 ; c1ccc(-c2ccc(N(c3ccccc3-c3ccccc3)c3cccc4c3Oc3cc5ccccc5cc3-c3ccc(-c5ccccc5)cc3-4)cc2)cc1 ; c1ccc(-c2ccc(-c3ccc(N(c4ccccc4)c4cccc5c4Oc4cc6ccccc6cc4-c4ccc(-c6ccc(-c7ccccc7)cc6)cc4-5)cc3)cc2)cc1]

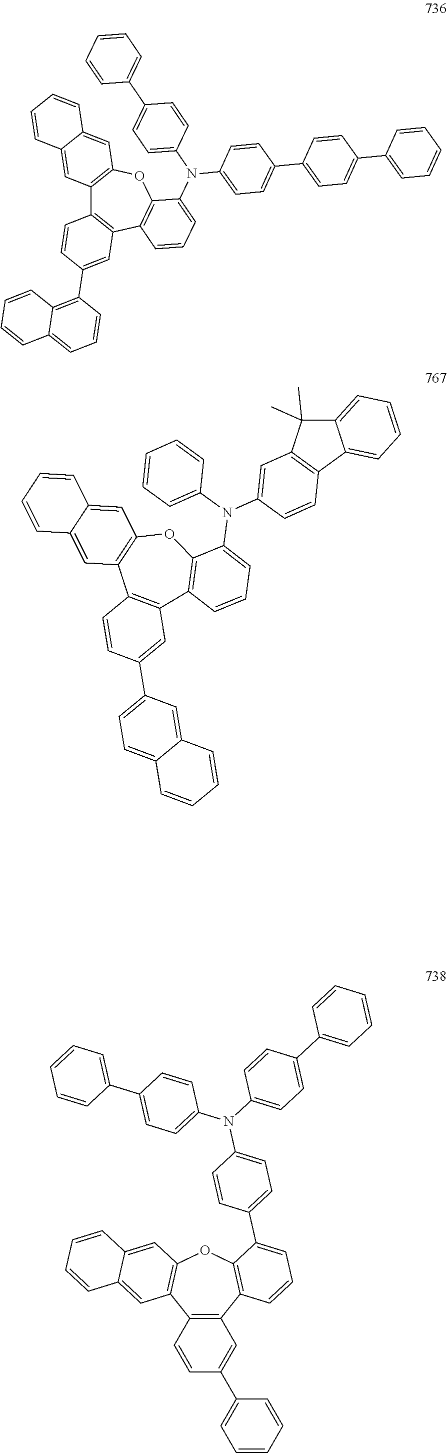 [SMILES: c1ccc(-c2ccc(-c3ccc(N(c4ccc(-c5ccccc5)cc4)c4cccc5c4Oc4cc6ccccc6cc4-c4ccc(-c6cccc7ccccc67)cc4-5)cc3)cc2)cc1 ; CC1(C)c2ccccc2-c2ccc(N(c3ccccc3)c3cccc4c3Oc3cc5ccccc5cc3-c3ccc(-c5ccc6ccccc6c5)cc3-4)cc21 ; c1ccc(-c2ccc(N(c3ccc(-c4ccccc4)cc3)c3ccc(-c4cccc5c4Oc4cc6ccccc6cc4-c4ccc(-c6ccccc6)cc4-5)cc3)cc2)cc1]

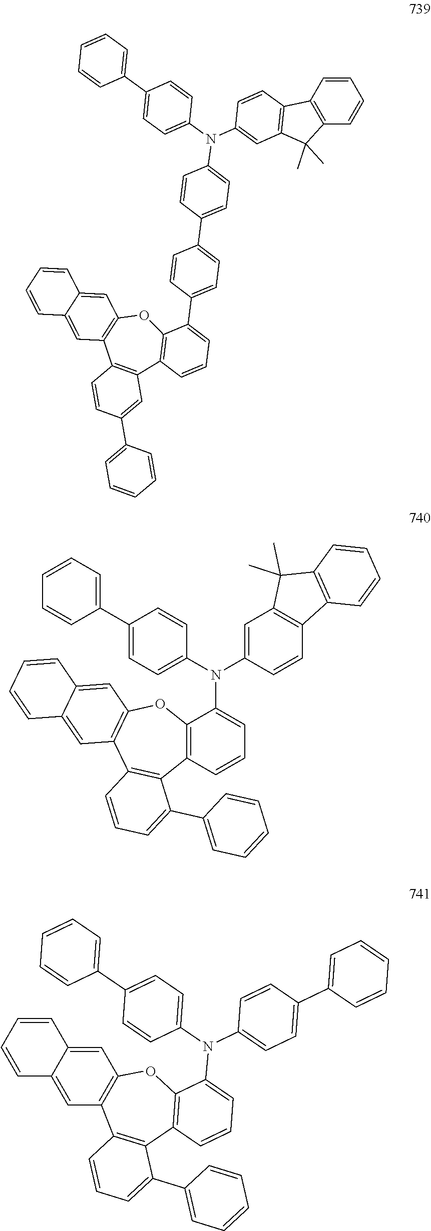 [SMILES: CC1(C)c2ccccc2-c2ccc(N(c3ccc(-c4ccccc4)cc3)c3ccc(-c4ccc(-c5cccc6c5Oc5cc7ccccc7cc5-c5ccc(-c7ccccc7)cc5-6)cc4)cc3)cc21 ; CC1(C)c2ccccc2-c2ccc(N(c3ccc(-c4ccccc4)cc3)c3cccc4c3Oc3cc5ccccc5cc3-c3cccc(-c5ccccc5)c3-4)cc21 ; c1ccc(-c2ccc(N(c3ccc(-c4ccccc4)cc3)c3cccc4c3Oc3cc5ccccc5cc3-c3cccc(-c5ccccc5)c3-4)cc2)cc1]

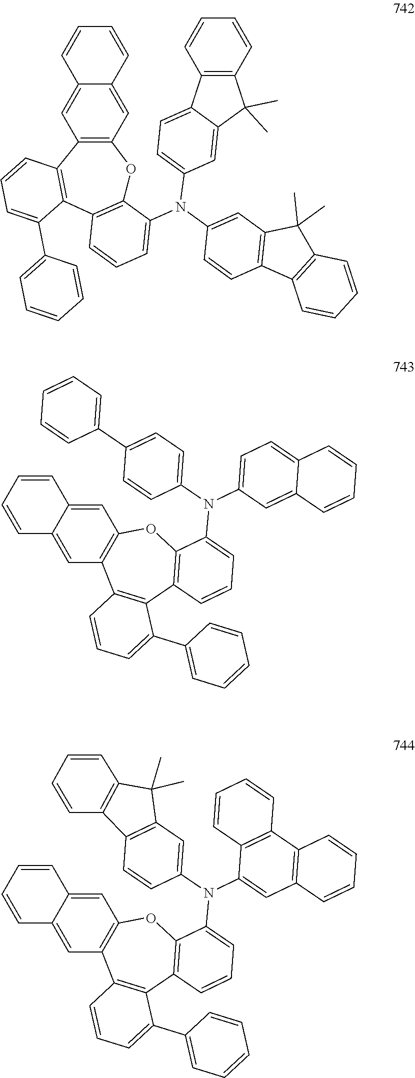 [SMILES: CC1(C)c2ccccc2-c2ccc(N(c3ccc4c(c3)C(C)(C)c3ccccc3-4)c3cccc4c3Oc3cc5ccccc5cc3-c3cccc(-c5ccccc5)c3-4)cc21 ; c1ccc(-c2ccc(N(c3ccc4ccccc4c3)c3cccc4c3Oc3cc5ccccc5cc3-c3cccc(-c5ccccc5)c3-4)cc2)cc1 ; CC1(C)c2ccccc2-c2ccc(N(c3cccc4c3Oc3cc5ccccc5cc3-c3cccc(-c5ccccc5)c3-4)c3cc4ccccc4c4ccccc34)cc21]

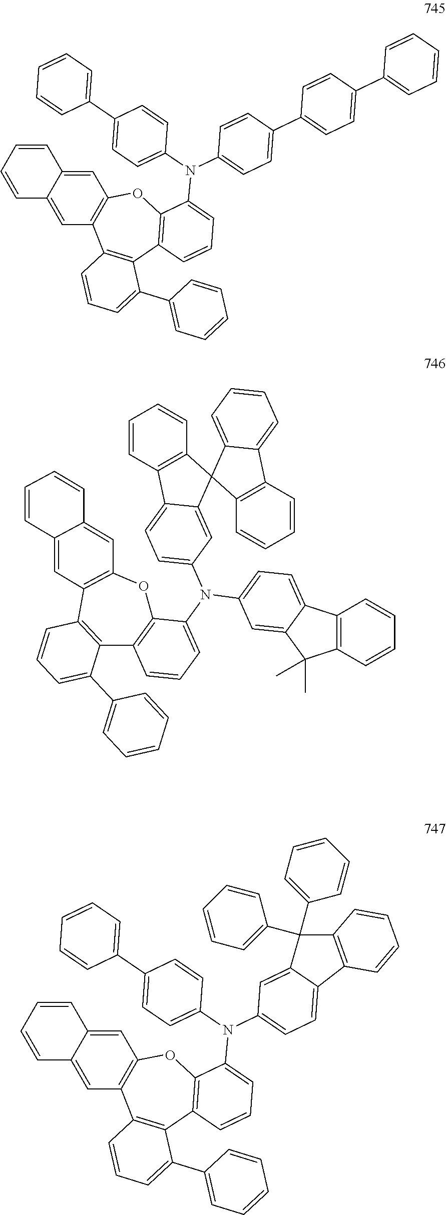 [SMILES: c1ccc(-c2ccc(-c3ccc(N(c4ccc(-c5ccccc5)cc4)c4cccc5c4Oc4cc6ccccc6cc4-c4cccc(-c6ccccc6)c4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cc5ccccc5cc3-c3cccc(-c5ccccc5)c3-4)cc21 ; c1ccc(-c2ccc(N(c3ccc4c(c3)C(c3ccccc3)(c3ccccc3)c3ccccc3-4)c3cccc4c3Oc3cc5ccccc5cc3-c3cccc(-c5ccccc5)c3-4)cc2)cc1]

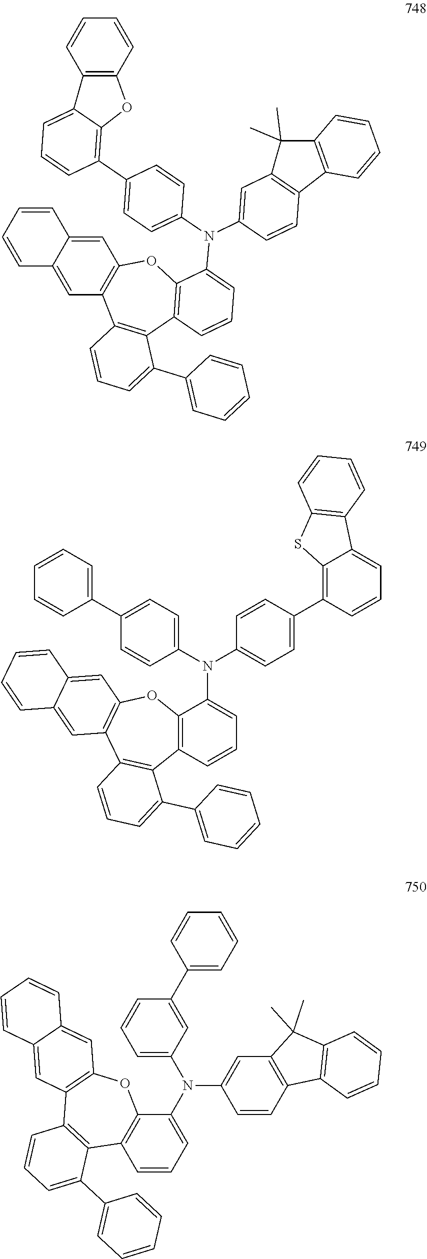 [SMILES: CC1(C)c2ccccc2-c2ccc(N(c3ccc(-c4cccc5c4oc4ccccc45)cc3)c3cccc4c3Oc3cc5ccccc5cc3-c3cccc(-c5ccccc5)c3-4)cc21 ; c1ccc(-c2ccc(N(c3ccc(-c4cccc5c4sc4ccccc45)cc3)c3cccc4c3Oc3cc5ccccc5cc3-c3cccc(-c5ccccc5)c3-4)cc2)cc1 ; CC1(C)c2ccccc2-c2ccc(N(c3cccc(-c4ccccc4)c3)c3cccc4c3Oc3cc5ccccc5cc3-c3cccc(-c5ccccc5)c3-4)cc21]

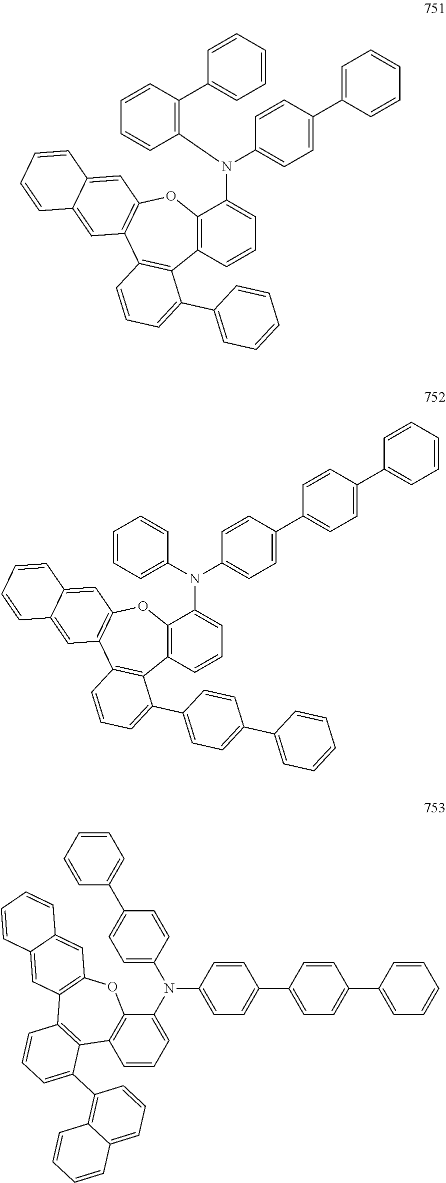 [SMILES: c1ccc(-c2ccc(N(c3ccccc3-c3ccccc3)c3cccc4c3Oc3cc5ccccc5cc3-c3cccc(-c5ccccc5)c3-4)cc2)cc1 ; c1ccc(-c2ccc(-c3ccc(N(c4ccccc4)c4cccc5c4Oc4cc6ccccc6cc4-c4cccc(-c6ccc(-c7ccccc7)cc6)c4-5)cc3)cc2)cc1 ; c1ccc(-c2ccc(-c3ccc(N(c4ccc(-c5ccccc5)cc4)c4cccc5c4Oc4cc6ccccc6cc4-c4cccc(-c6cccc7ccccc67)c4-5)cc3)cc2)cc1]

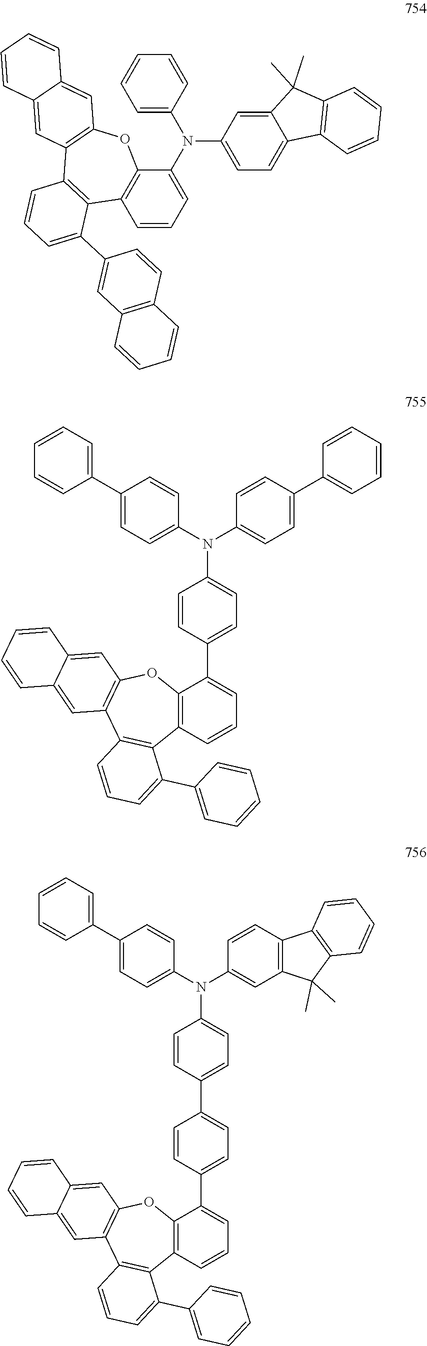 [SMILES: CC1(C)c2ccccc2-c2ccc(N(c3ccccc3)c3cccc4c3Oc3cc5ccccc5cc3-c3cccc(-c5ccc6ccccc6c5)c3-4)cc21 ; c1ccc(-c2ccc(N(c3ccc(-c4ccccc4)cc3)c3ccc(-c4cccc5c4Oc4cc6ccccc6cc4-c4cccc(-c6ccccc6)c4-5)cc3)cc2)cc1 ; CC1(C)c2ccccc2-c2ccc(N(c3ccc(-c4ccccc4)cc3)c3ccc(-c4ccc(-c5cccc6c5Oc5cc7ccccc7cc5-c5cccc(-c7ccccc7)c5-6)cc4)cc3)cc21]

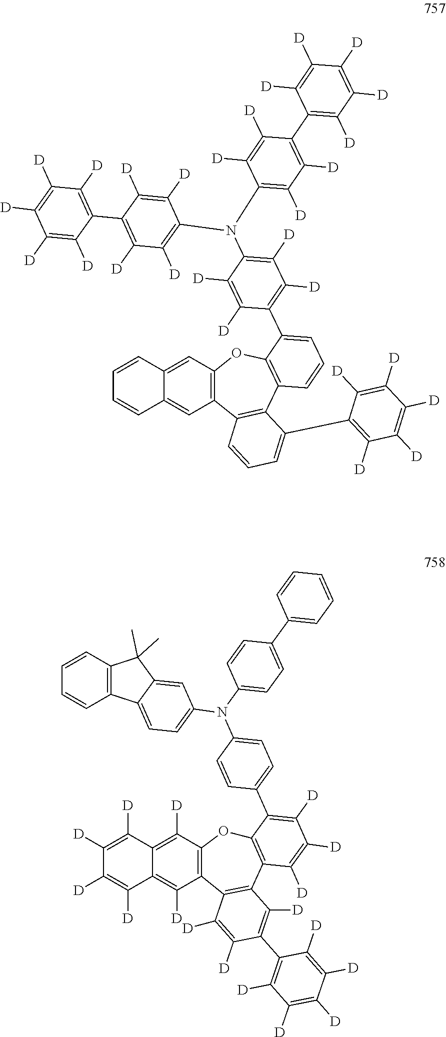 [SMILES: [2H]c1c([2H])c([2H])c(-c2cccc3c2-c2cccc(-c4c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c4[2H])c2Oc2cc4ccccc4cc2-3)c([2H])c1[2H] ; [2H]c1c([2H])c([2H])c(-c2c([2H])c([2H])c3c(c2[2H])-c2c([2H])c([2H])c([2H])c(-c4ccc(N(c5ccc(-c6ccccc6)cc5)c5ccc6c(c5)C(C)(C)c5ccccc5-6)cc4)c2Oc2c-3c([2H])c3c([2H])c([2H])c([2H])c([2H])c3c2[2H])c([2H])c1[2H]]

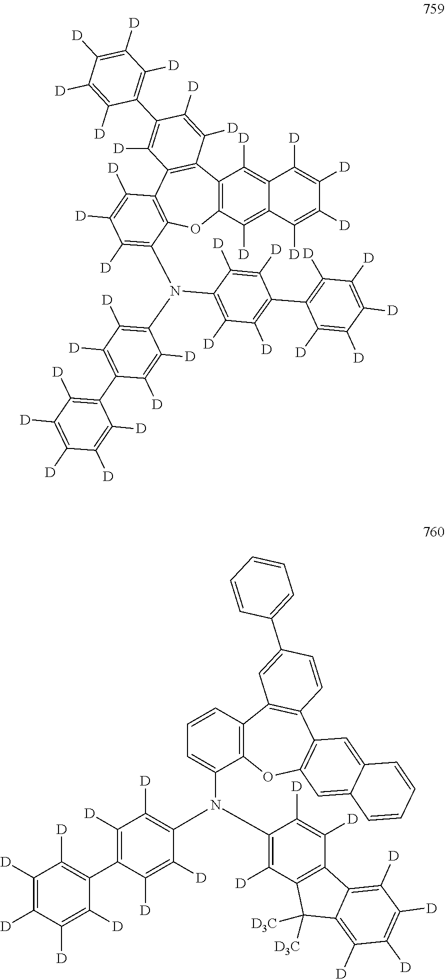

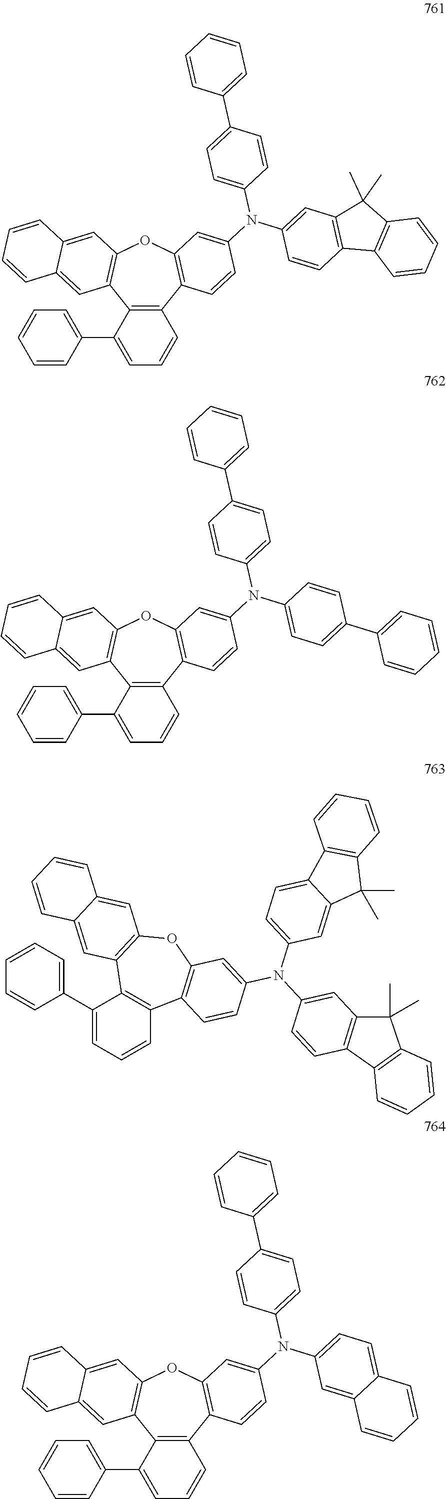 [SMILES: CC1(C)c2ccccc2-c2ccc(N(c3ccc(-c4ccccc4)cc3)c3ccc4c(c3)Oc3cc5ccccc5cc3-c3c(-c5ccccc5)cccc3-4)cc21 ; c1ccc(-c2ccc(N(c3ccc(-c4ccccc4)cc3)c3ccc4c(c3)Oc3cc5ccccc5cc3-c3c(-c5ccccc5)cccc3-4)cc2)cc1 ; CC1(C)c2ccccc2-c2ccc(N(c3ccc4c(c3)Oc3cc5ccccc5cc3-c3c(-c5ccccc5)cccc3-4)c3ccc4c(c3)C(C)(C)c3ccccc3-4)cc21 ; c1ccc(-c2ccc(N(c3ccc4c(c3)Oc3cc5ccccc5cc3-c3c(-c5ccccc5)cccc3-4)c3ccc4ccccc4c3)cc2)cc1]

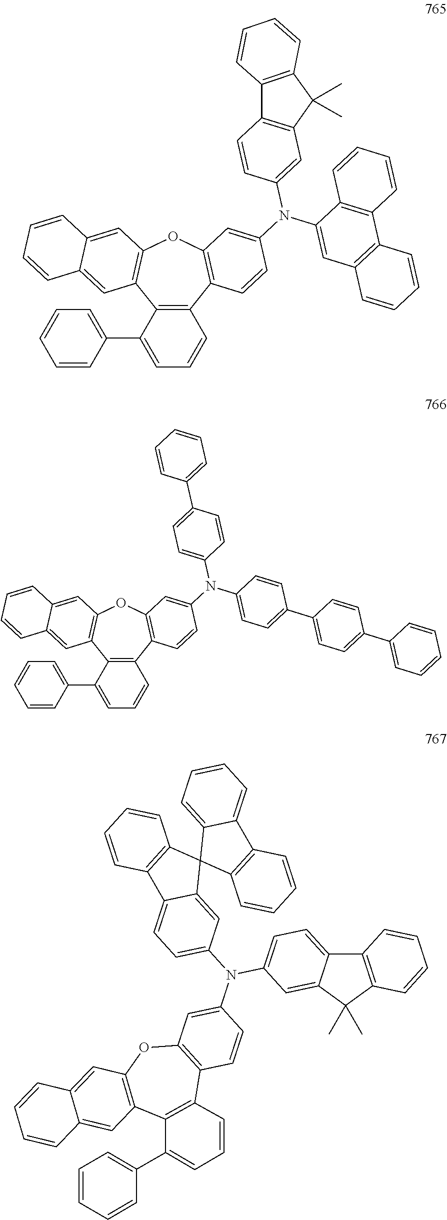 [SMILES: CC1(C)c2ccccc2-c2ccc(N(c3ccc4c(c3)Oc3cc5ccccc5cc3-c3c(-c5ccccc5)cccc3-4)c3cc4ccccc4c4ccccc34)cc21 ; c1ccc(-c2ccc(-c3ccc(N(c4ccc(-c5ccccc5)cc4)c4ccc5c(c4)Oc4cc6ccccc6cc4-c4c(-c6ccccc6)cccc4-5)cc3)cc2)cc1 ; CC1(C)c2ccccc2-c2ccc(N(c3ccc4c(c3)Oc3cc5ccccc5cc3-c3c(-c5ccccc5)cccc3-4)c3ccc4c(c3)C3(c5ccccc5-c5ccccc53)c3ccccc3-4)cc21]

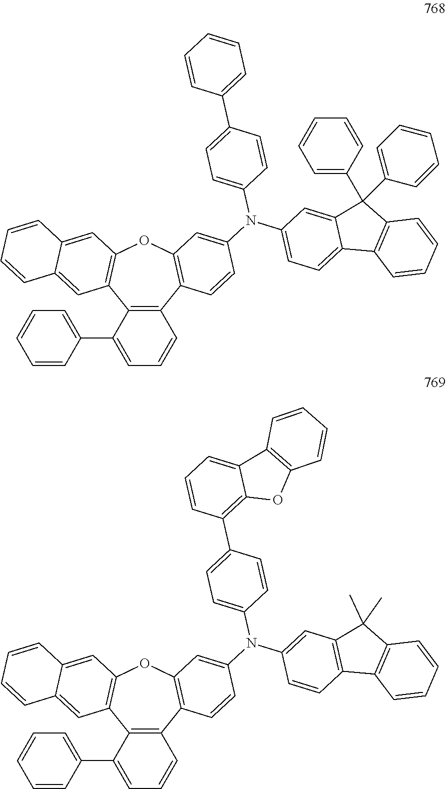 [SMILES: c1ccc(-c2ccc(N(c3ccc4c(c3)Oc3cc5ccccc5cc3-c3c(-c5ccccc5)cccc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc5ccccc5cc3-c3c(-c5ccccc5)cccc3-4)cc21]

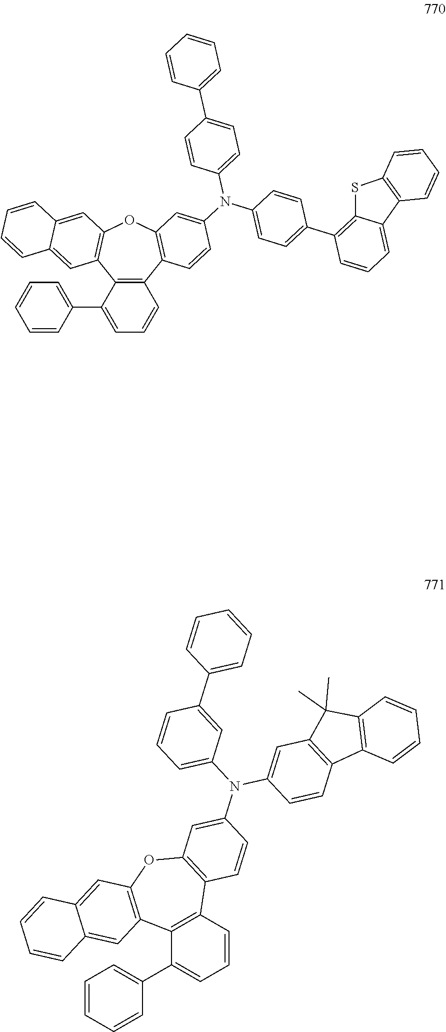 [SMILES: c1ccc(-c2ccc(N(c3ccc(-c4cccc5c4sc4ccccc45)cc3)c3ccc4c(c3)Oc3cc5ccccc5cc3-c3c(-c5ccccc5)cccc3-4)cc2)cc1 ; CC1(C)c2ccccc2-c2ccc(N(c3cccc(-c4ccccc4)c3)c3ccc4c(c3)Oc3cc5ccccc5cc3-c3c(-c5ccccc5)cccc3-4)cc21]

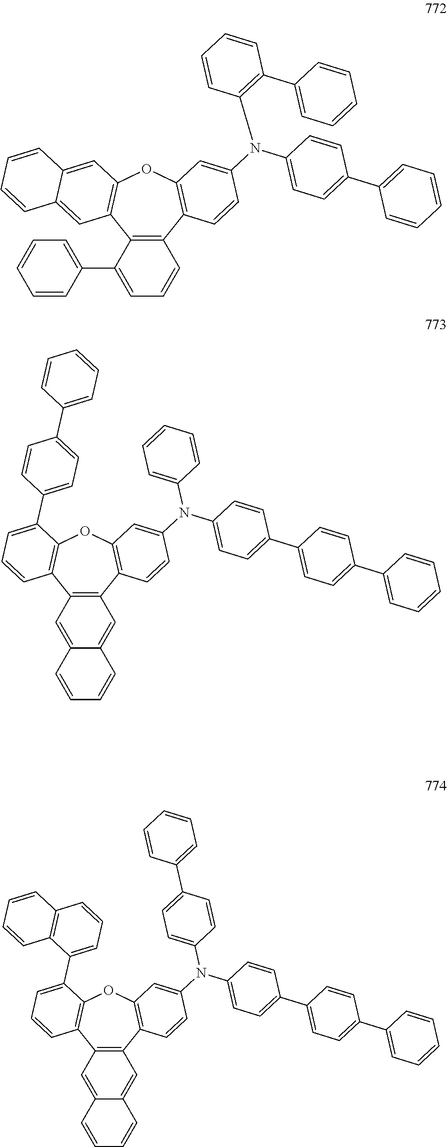 [SMILES: c1ccc(-c2ccc(N(c3ccc4c(c3)Oc3cc5ccccc5cc3-c3c(-c5ccccc5)cccc3-4)c3ccccc3-c3ccccc3)cc2)cc1 ; c1ccc(-c2ccc(-c3ccc(N(c4ccccc4)c4ccc5c(c4)Oc4c(-c6ccc(-c7ccccc7)cc6)cccc4-c4cc6ccccc6cc4-5)cc3)cc2)cc1 ; c1ccc(-c2ccc(-c3ccc(N(c4ccc(-c5ccccc5)cc4)c4ccc5c(c4)Oc4c(cccc4-c4cccc6ccccc46)-c4cc6ccccc6cc4-5)cc3)cc2)cc1]

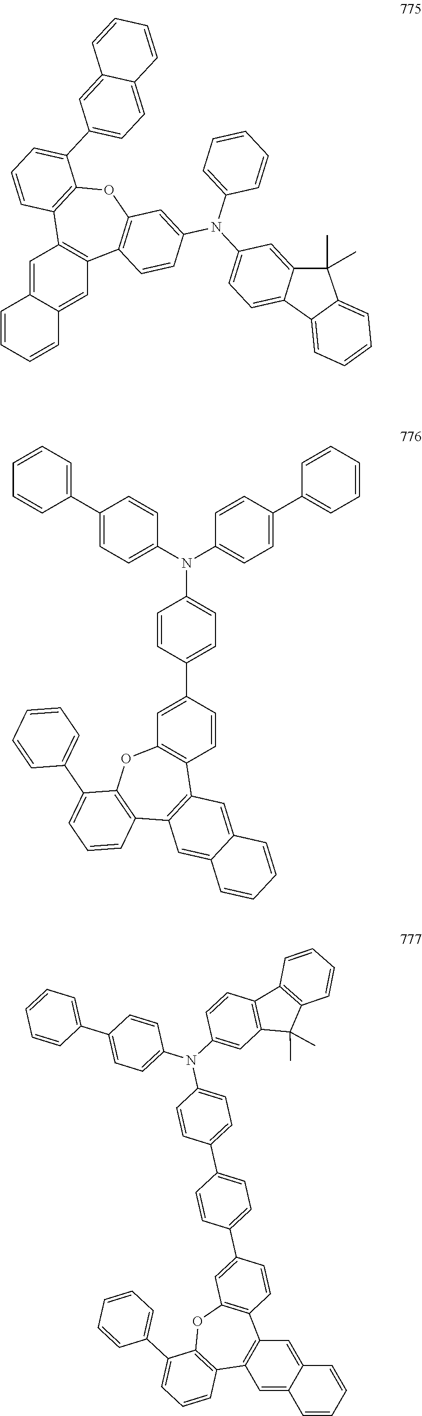 [SMILES: CC1(C)c2ccccc2-c2ccc(N(c3ccccc3)c3ccc4c(c3)Oc3c(-c5ccc6ccccc6c5)cccc3-c3cc5ccccc5cc3-4)cc21 ; c1ccc(-c2ccc(N(c3ccc(-c4ccccc4)cc3)c3ccc(-c4ccc5c(c4)Oc4c(-c6ccccc6)cccc4-c4cc6ccccc6cc4-5)cc3)cc2)cc1 ; CC1(C)c2ccccc2-c2ccc(N(c3ccc(-c4ccccc4)cc3)c3ccc(-c4ccc(-c5ccc6c(c5)Oc5c(-c7ccccc7)cccc5-c5cc7ccccc7cc5-6)cc4)cc3)cc21]

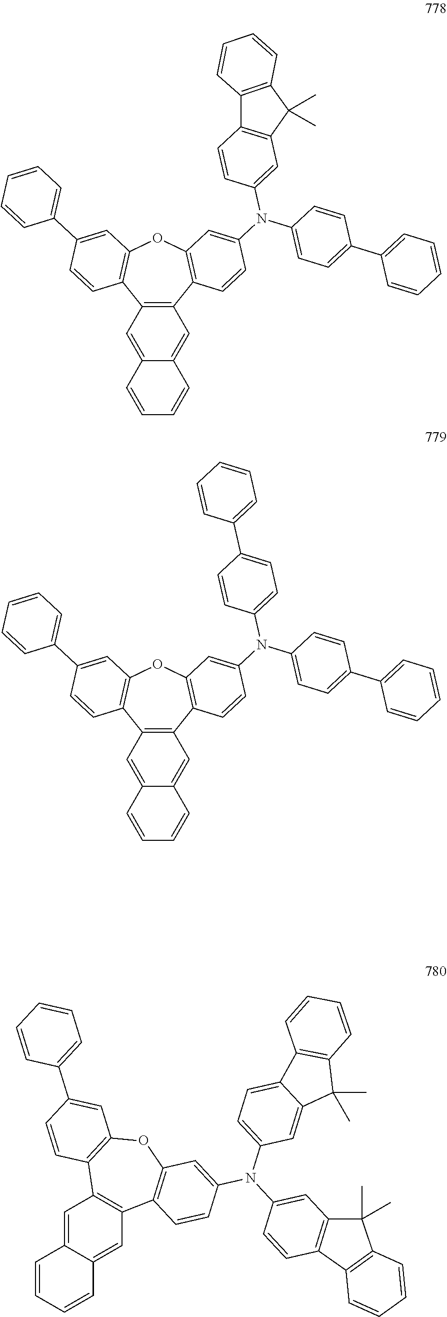 [SMILES: CC1(C)c2ccccc2-c2ccc(N(c3ccc(-c4ccccc4)cc3)c3ccc4c(c3)Oc3cc(-c5ccccc5)ccc3-c3cc5ccccc5cc3-4)cc21 ; c1ccc(-c2ccc(N(c3ccc(-c4ccccc4)cc3)c3ccc4c(c3)Oc3cc(-c5ccccc5)ccc3-c3cc5ccccc5cc3-4)cc2)cc1 ; CC1(C)c2ccccc2-c2ccc(N(c3ccc4c(c3)Oc3cc(-c5ccccc5)ccc3-c3cc5ccccc5cc3-4)c3ccc4c(c3)C(C)(C)c3ccccc3-4)cc21]

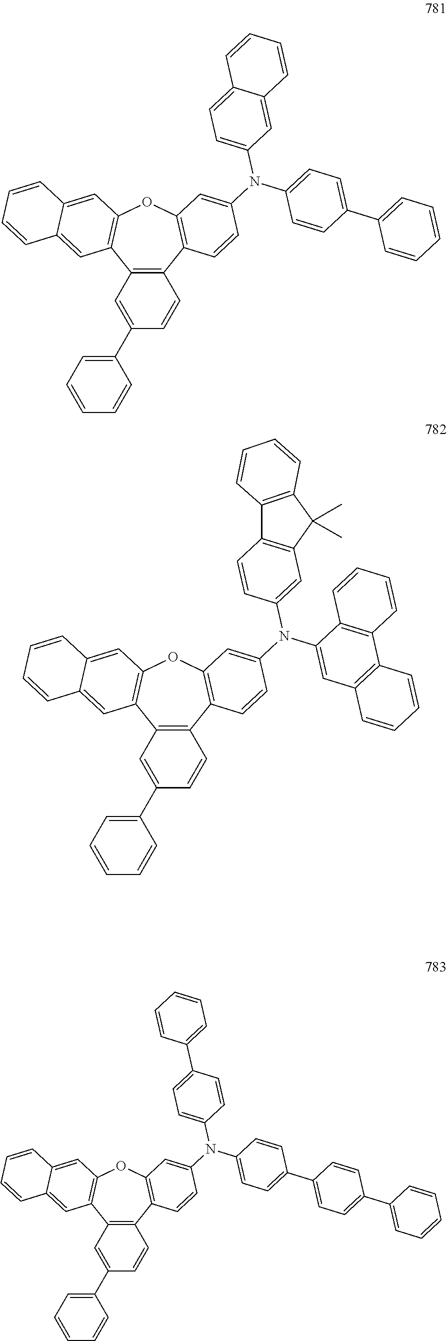 [SMILES: c1ccc(-c2ccc(N(c3ccc4c(c3)Oc3cc5ccccc5cc3-c3cc(-c5ccccc5)ccc3-4)c3ccc4ccccc4c3)cc2)cc1 ; CC1(C)c2ccccc2-c2ccc(N(c3ccc4c(c3)Oc3cc5ccccc5cc3-c3cc(-c5ccccc5)ccc3-4)c3cc4ccccc4c4ccccc34)cc21 ; c1ccc(-c2ccc(-c3ccc(N(c4ccc(-c5ccccc5)cc4)c4ccc5c(c4)Oc4cc6ccccc6cc4-c4cc(-c6ccccc6)ccc4-5)cc3)cc2)cc1]

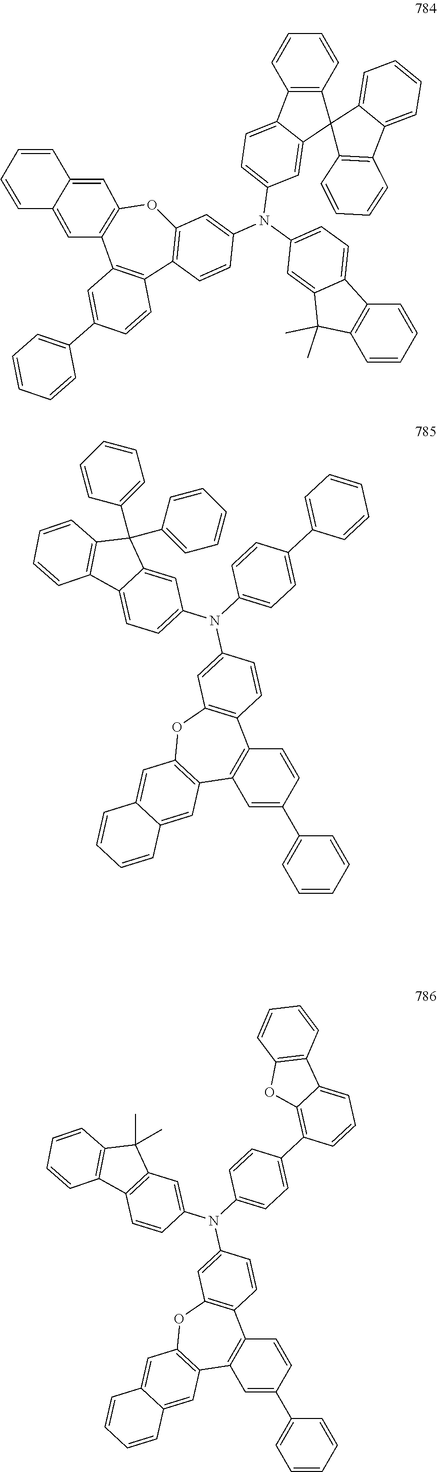 [SMILES: CC1(C)c2ccccc2-c2ccc(N(c3ccc4c(c3)Oc3cc5ccccc5cc3-c3cc(-c5ccccc5)ccc3-4)c3ccc4c(c3)C3(c5ccccc5-c5ccccc53)c3ccccc3-4)cc21 ; c1ccc(-c2ccc(N(c3ccc4c(c3)Oc3cc5ccccc5cc3-c3cc(-c5ccccc5)ccc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc5ccccc5cc3-c3cc(-c5ccccc5)ccc3-4)cc21]

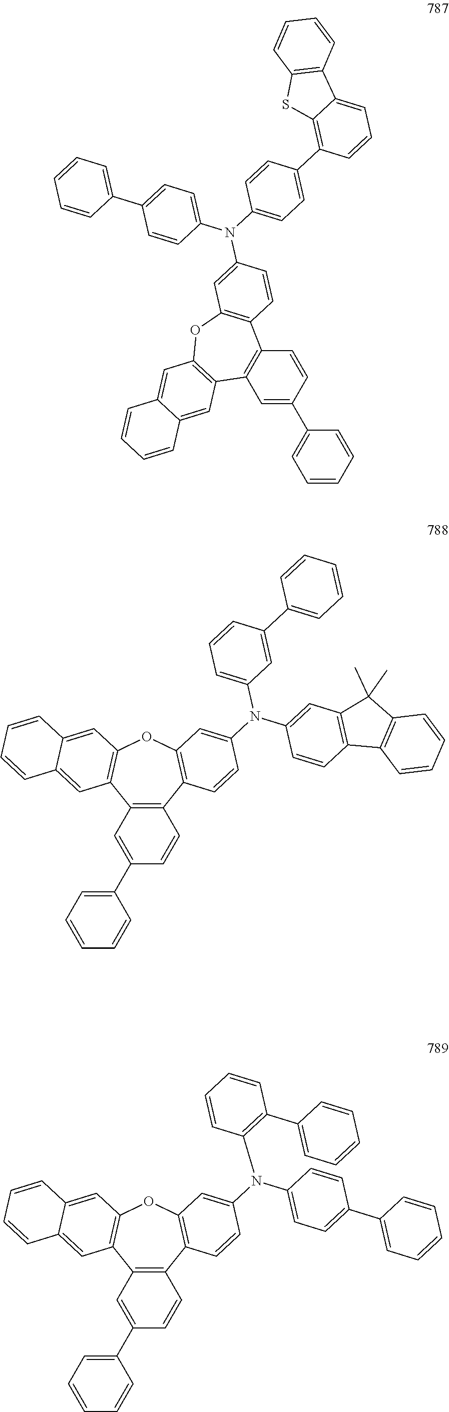 [SMILES: c1ccc(-c2ccc(N(c3ccc(-c4cccc5c4sc4ccccc45)cc3)c3ccc4c(c3)Oc3cc5ccccc5cc3-c3cc(-c5ccccc5)ccc3-4)cc2)cc1 ; CC1(C)c2ccccc2-c2ccc(N(c3cccc(-c4ccccc4)c3)c3ccc4c(c3)Oc3cc5ccccc5cc3-c3cc(-c5ccccc5)ccc3-4)cc21 ; c1ccc(-c2ccc(N(c3ccc4c(c3)Oc3cc5ccccc5cc3-c3cc(-c5ccccc5)ccc3-4)c3ccccc3-c3ccccc3)cc2)cc1]

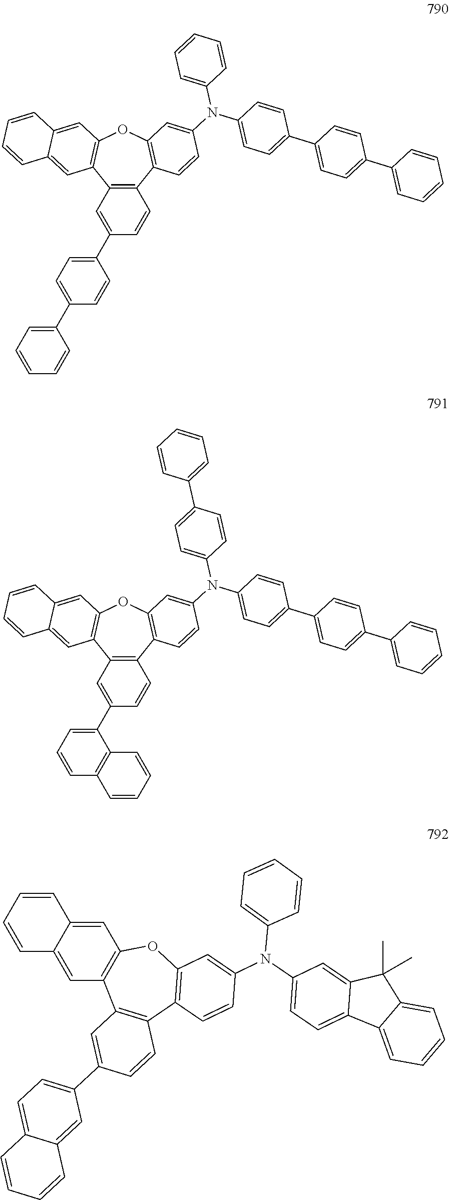 [SMILES: c1ccc(-c2ccc(-c3ccc(N(c4ccccc4)c4ccc5c(c4)Oc4cc6ccccc6cc4-c4cc(-c6ccc(-c7ccccc7)cc6)ccc4-5)cc3)cc2)cc1 ; c1ccc(-c2ccc(-c3ccc(N(c4ccc(-c5ccccc5)cc4)c4ccc5c(c4)Oc4cc6ccccc6cc4-c4cc(-c6cccc7ccccc67)ccc4-5)cc3)cc2)cc1 ; CC1(C)c2ccccc2-c2ccc(N(c3ccccc3)c3ccc4c(c3)Oc3cc5ccccc5cc3-c3cc(-c5ccc6ccccc6c5)ccc3-4)cc21]

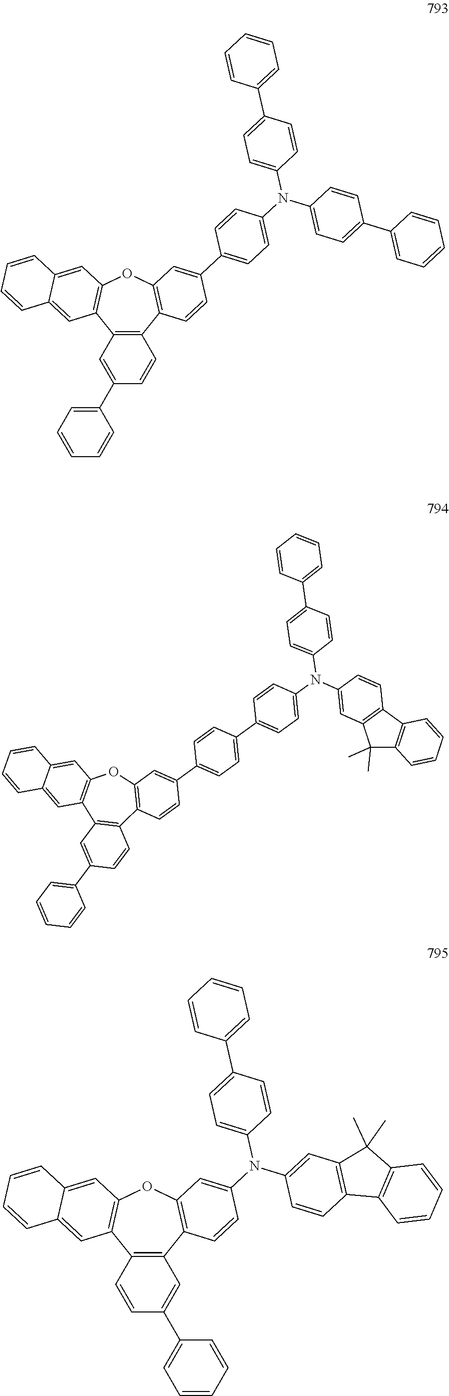 [SMILES: c1ccc(-c2ccc(N(c3ccc(-c4ccccc4)cc3)c3ccc(-c4ccc5c(c4)Oc4cc6ccccc6cc4-c4cc(-c6ccccc6)ccc4-5)cc3)cc2)cc1 ; CC1(C)c2ccccc2-c2ccc(N(c3ccc(-c4ccccc4)cc3)c3ccc(-c4ccc(-c5ccc6c(c5)Oc5cc7ccccc7cc5-c5cc(-c7ccccc7)ccc5-6)cc4)cc3)cc21 ; CC1(C)c2ccccc2-c2ccc(N(c3ccc(-c4ccccc4)cc3)c3ccc4c(c3)Oc3cc5ccccc5cc3-c3ccc(-c5ccccc5)cc3-4)cc21]

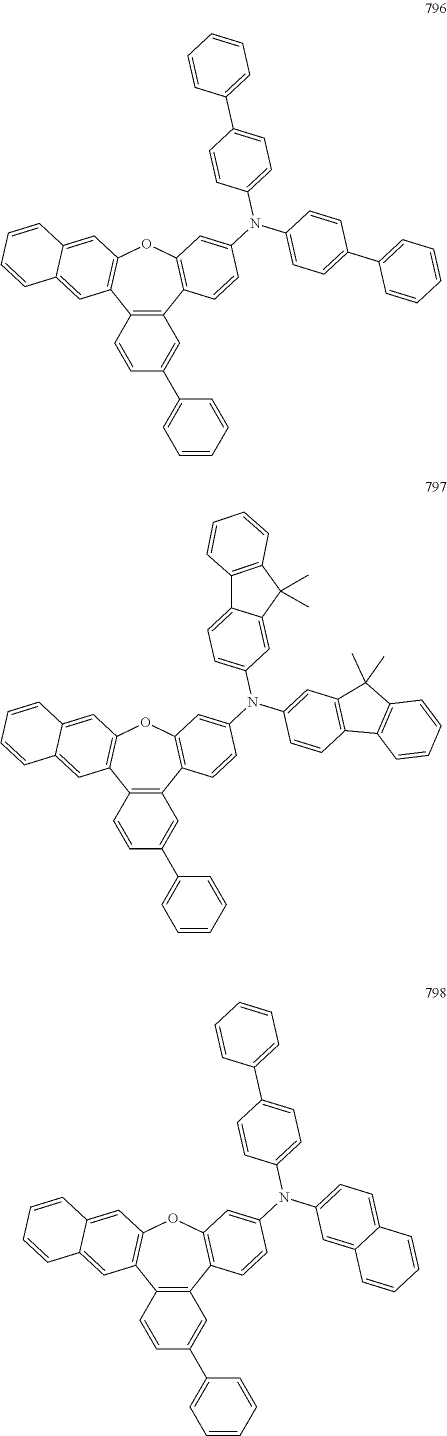 [SMILES: c1ccc(-c2ccc(N(c3ccc(-c4ccccc4)cc3)c3ccc4c(c3)Oc3cc5ccccc5cc3-c3ccc(-c5ccccc5)cc3-4)cc2)cc1 ; CC1(C)c2ccccc2-c2ccc(N(c3ccc4c(c3)Oc3cc5ccccc5cc3-c3ccc(-c5ccccc5)cc3-4)c3ccc4c(c3)C(C)(C)c3ccccc3-4)cc21 ; c1ccc(-c2ccc(N(c3ccc4c(c3)Oc3cc5ccccc5cc3-c3ccc(-c5ccccc5)cc3-4)c3ccc4ccccc4c3)cc2)cc1]

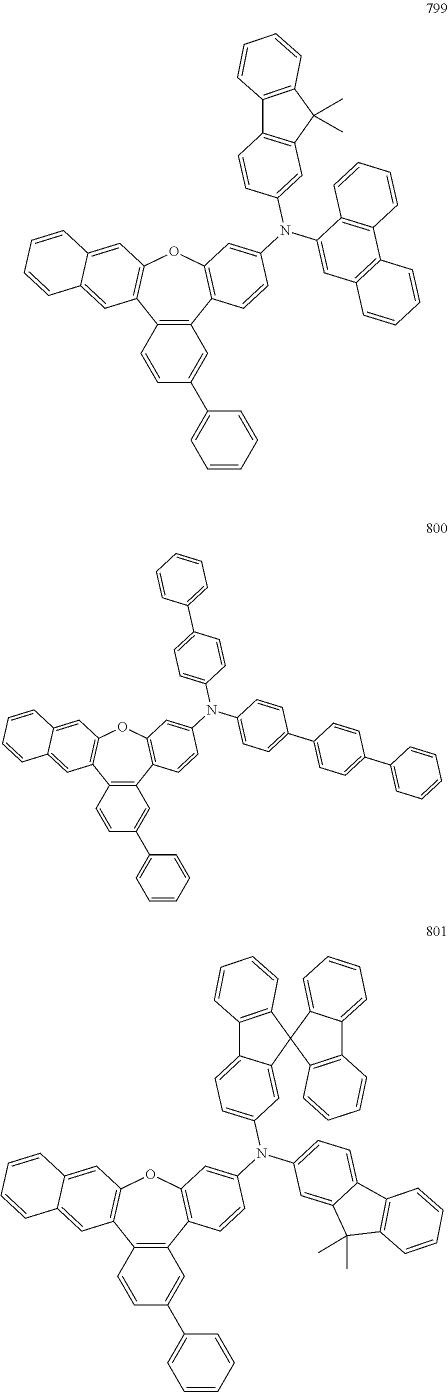 [SMILES: CC1(C)c2ccccc2-c2ccc(N(c3ccc4c(c3)Oc3cc5ccccc5cc3-c3ccc(-c5ccccc5)cc3-4)c3cc4ccccc4c4ccccc34)cc21 ; c1ccc(-c2ccc(-c3ccc(N(c4ccc(-c5ccccc5)cc4)c4ccc5c(c4)Oc4cc6ccccc6cc4-c4ccc(-c6ccccc6)cc4-5)cc3)cc2)cc1 ; CC1(C)c2ccccc2-c2ccc(N(c3ccc4c(c3)Oc3cc5ccccc5cc3-c3ccc(-c5ccccc5)cc3-4)c3ccc4c(c3)C3(c5ccccc5-c5ccccc53)c3ccccc3-4)cc21]

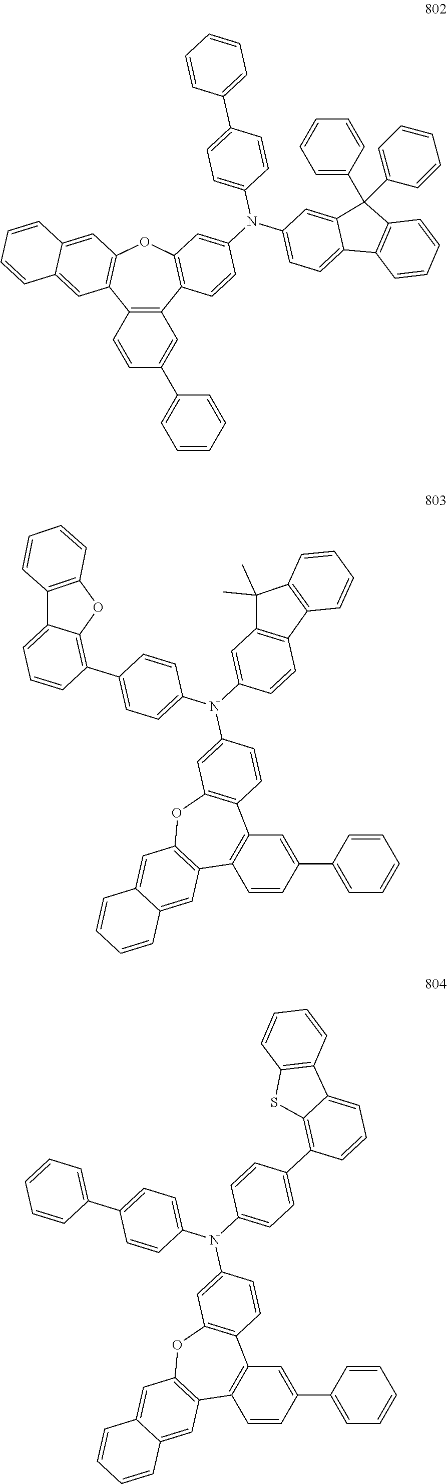 [SMILES: c1ccc(-c2ccc(N(c3ccc4c(c3)Oc3cc5ccccc5cc3-c3ccc(-c5ccccc5)cc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc5ccccc5cc3-c3ccc(-c5ccccc5)cc3-4)cc21 ; c1ccc(-c2ccc(N(c3ccc(-c4cccc5c4sc4ccccc45)cc3)c3ccc4c(c3)Oc3cc5ccccc5cc3-c3ccc(-c5ccccc5)cc3-4)cc2)cc1]

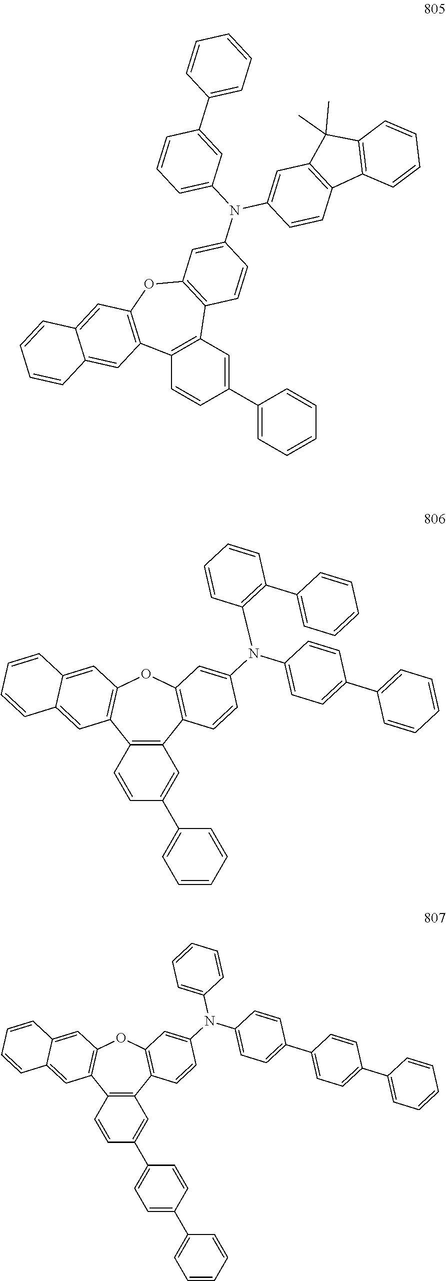 [SMILES: CC1(C)c2ccccc2-c2ccc(N(c3cccc(-c4ccccc4)c3)c3ccc4c(c3)Oc3cc5ccccc5cc3-c3ccc(-c5ccccc5)cc3-4)cc21 ; c1ccc(-c2ccc(N(c3ccc4c(c3)Oc3cc5ccccc5cc3-c3ccc(-c5ccccc5)cc3-4)c3ccccc3-c3ccccc3)cc2)cc1 ; c1ccc(-c2ccc(-c3ccc(N(c4ccccc4)c4ccc5c(c4)Oc4cc6ccccc6cc4-c4ccc(-c6ccc(-c7ccccc7)cc6)cc4-5)cc3)cc2)cc1]

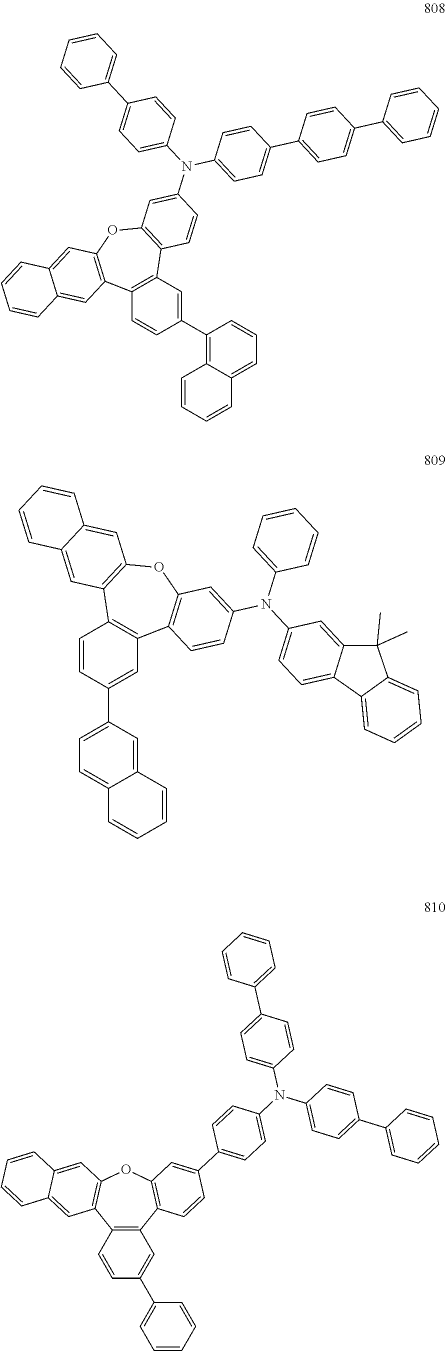 [SMILES: c1ccc(-c2ccc(-c3ccc(N(c4ccc(-c5ccccc5)cc4)c4ccc5c(c4)Oc4cc6ccccc6cc4-c4ccc(-c6cccc7ccccc67)cc4-5)cc3)cc2)cc1 ; CC1(C)c2ccccc2-c2ccc(N(c3ccccc3)c3ccc4c(c3)Oc3cc5ccccc5cc3-c3ccc(-c5ccc6ccccc6c5)cc3-4)cc21 ; c1ccc(-c2ccc(N(c3ccc(-c4ccccc4)cc3)c3ccc(-c4ccc5c(c4)Oc4cc6ccccc6cc4-c4ccc(-c6ccccc6)cc4-5)cc3)cc2)cc1]

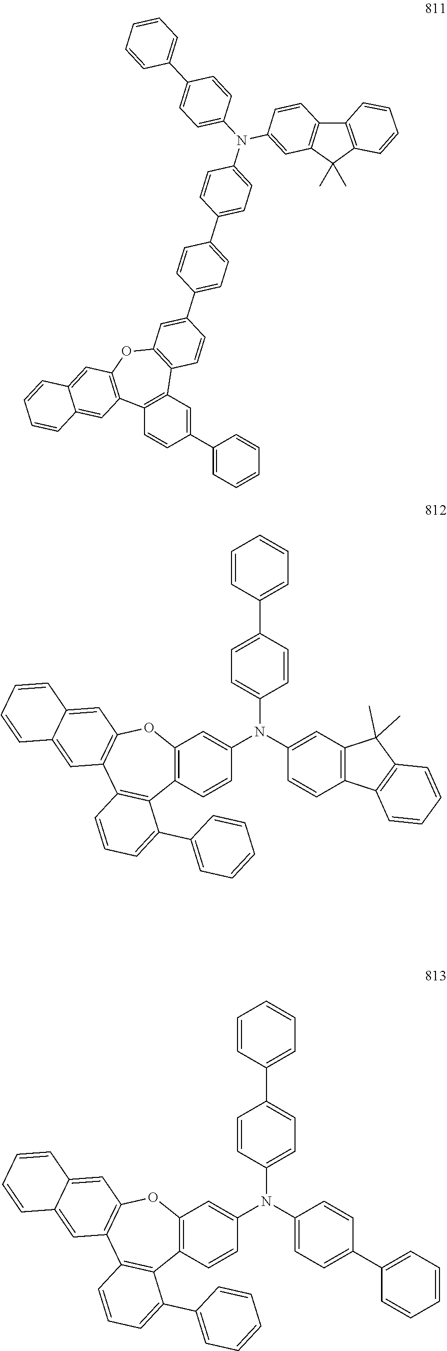 [SMILES: CC1(C)c2ccccc2-c2ccc(N(c3ccc(-c4ccccc4)cc3)c3ccc(-c4ccc(-c5ccc6c(c5)Oc5cc7ccccc7cc5-c5ccc(-c7ccccc7)cc5-6)cc4)cc3)cc21 ; CC1(C)c2ccccc2-c2ccc(N(c3ccc(-c4ccccc4)cc3)c3ccc4c(c3)Oc3cc5ccccc5cc3-c3cccc(-c5ccccc5)c3-4)cc21 ; c1ccc(-c2ccc(N(c3ccc(-c4ccccc4)cc3)c3ccc4c(c3)Oc3cc5ccccc5cc3-c3cccc(-c5ccccc5)c3-4)cc2)cc1]

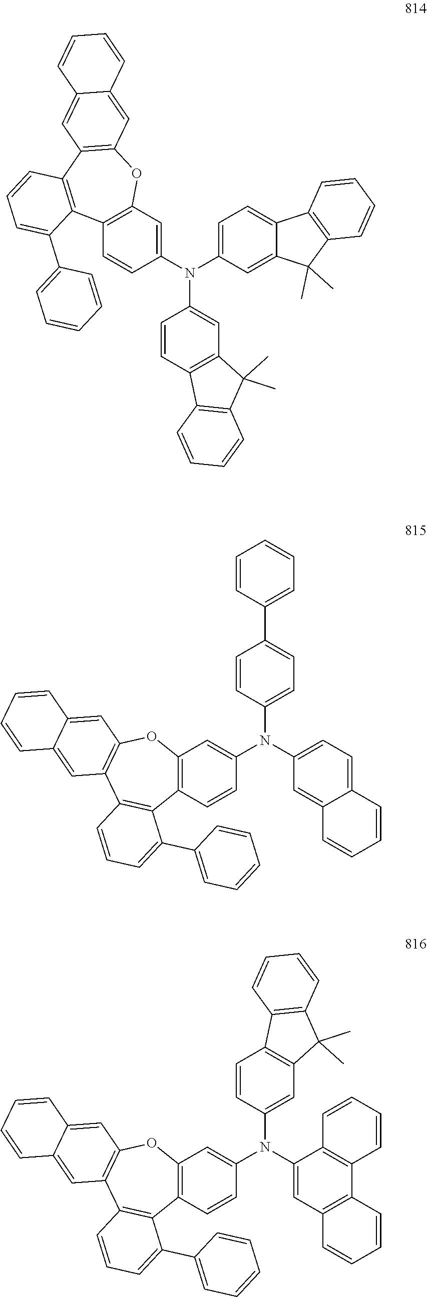 [SMILES: CC1(C)c2ccccc2-c2ccc(N(c3ccc4c(c3)Oc3cc5ccccc5cc3-c3cccc(-c5ccccc5)c3-4)c3ccc4c(c3)C(C)(C)c3ccccc3-4)cc21 ; c1ccc(-c2ccc(N(c3ccc4c(c3)Oc3cc5ccccc5cc3-c3cccc(-c5ccccc5)c3-4)c3ccc4ccccc4c3)cc2)cc1 ; CC1(C)c2ccccc2-c2ccc(N(c3ccc4c(c3)Oc3cc5ccccc5cc3-c3cccc(-c5ccccc5)c3-4)c3cc4ccccc4c4ccccc34)cc21]

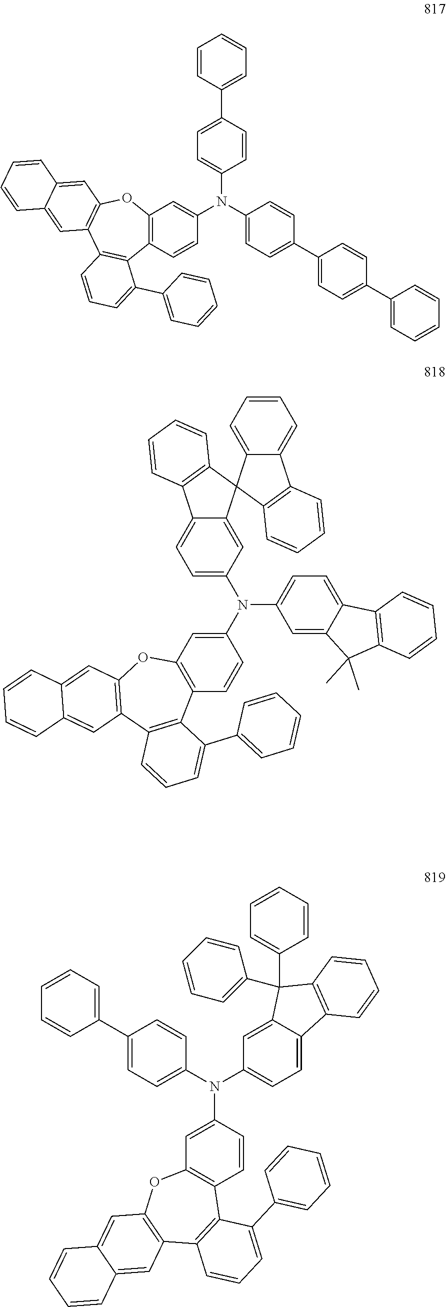 [SMILES: c1ccc(-c2ccc(-c3ccc(N(c4ccc(-c5ccccc5)cc4)c4ccc5c(c4)Oc4cc6ccccc6cc4-c4cccc(-c6ccccc6)c4-5)cc3)cc2)cc1 ; CC1(C)c2ccccc2-c2ccc(N(c3ccc4c(c3)Oc3cc5ccccc5cc3-c3cccc(-c5ccccc5)c3-4)c3ccc4c(c3)C3(c5ccccc5-c5ccccc53)c3ccccc3-4)cc21 ; c1ccc(-c2ccc(N(c3ccc4c(c3)Oc3cc5ccccc5cc3-c3cccc(-c5ccccc5)c3-4)c3ccc4c(c3)C(c3ccccc3)(c3ccccc3)c3ccccc3-4)cc2)cc1]

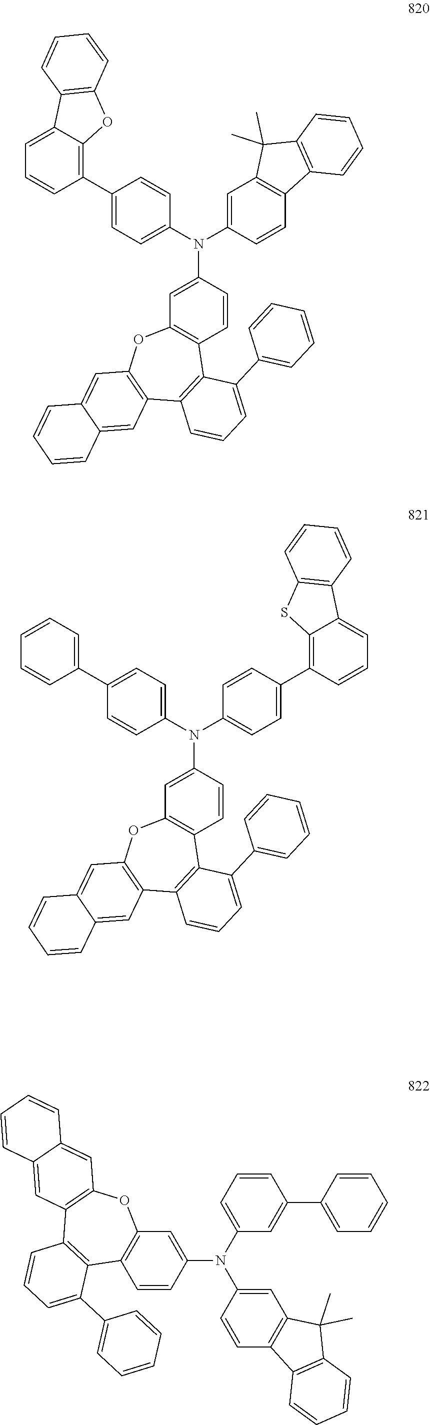 [SMILES: CC1(C)c2ccccc2-c2ccc(N(c3ccc(-c4cccc5c4oc4ccccc45)cc3)c3ccc4c(c3)Oc3cc5ccccc5cc3-c3cccc(-c5ccccc5)c3-4)cc21 ; c1ccc(-c2ccc(N(c3ccc(-c4cccc5c4sc4ccccc45)cc3)c3ccc4c(c3)Oc3cc5ccccc5cc3-c3cccc(-c5ccccc5)c3-4)cc2)cc1 ; CC1(C)c2ccccc2-c2ccc(N(c3cccc(-c4ccccc4)c3)c3ccc4c(c3)Oc3cc5ccccc5cc3-c3cccc(-c5ccccc5)c3-4)cc21]

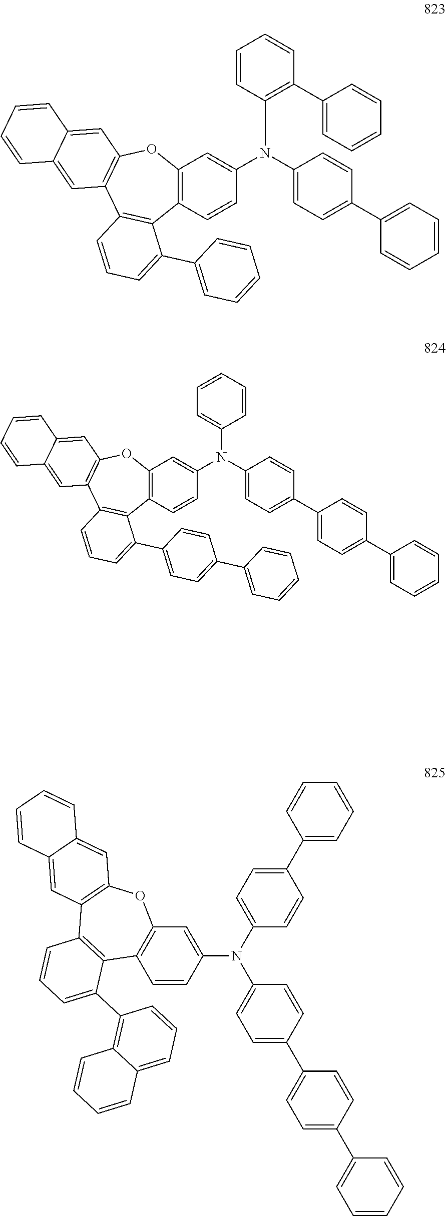 [SMILES: c1ccc(-c2ccc(N(c3ccc4c(c3)Oc3cc5ccccc5cc3-c3cccc(-c5ccccc5)c3-4)c3ccccc3-c3ccccc3)cc2)cc1 ; c1ccc(-c2ccc(-c3ccc(N(c4ccccc4)c4ccc5c(c4)Oc4cc6ccccc6cc4-c4cccc(-c6ccc(-c7ccccc7)cc6)c4-5)cc3)cc2)cc1 ; c1ccc(-c2ccc(-c3ccc(N(c4ccc(-c5ccccc5)cc4)c4ccc5c(c4)Oc4cc6ccccc6cc4-c4cccc(-c6cccc7ccccc67)c4-5)cc3)cc2)cc1]

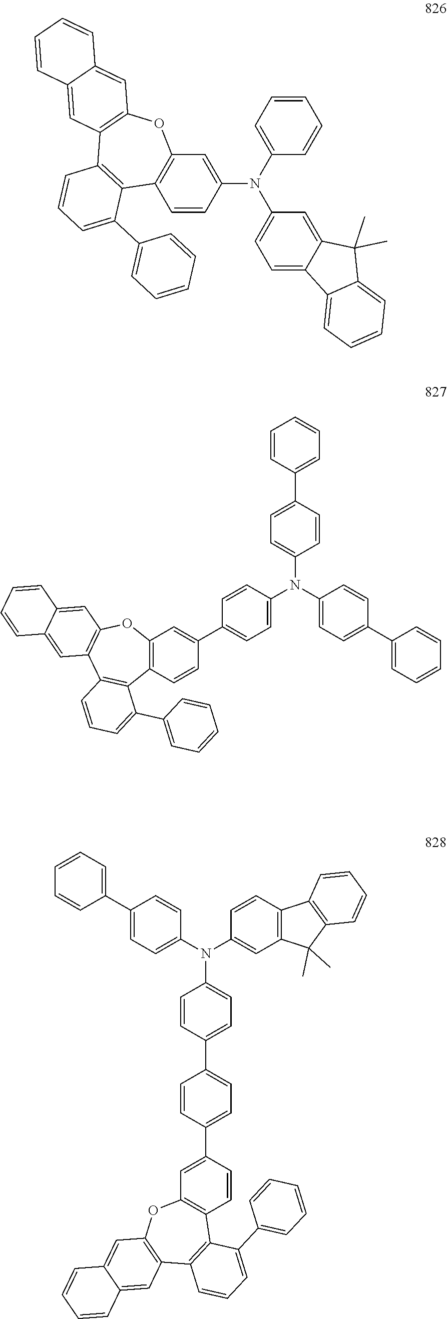 [SMILES: CC1(C)c2ccccc2-c2ccc(N(c3ccccc3)c3ccc4c(c3)Oc3cc5ccccc5cc3-c3cccc(-c5ccccc5)c3-4)cc21 ; c1ccc(-c2ccc(N(c3ccc(-c4ccccc4)cc3)c3ccc(-c4ccc5c(c4)Oc4cc6ccccc6cc4-c4cccc(-c6ccccc6)c4-5)cc3)cc2)cc1 ; CC1(C)c2ccccc2-c2ccc(N(c3ccc(-c4ccccc4)cc3)c3ccc(-c4ccc(-c5ccc6c(c5)Oc5cc7ccccc7cc5-c5cccc(-c7ccccc7)c5-6)cc4)cc3)cc21]

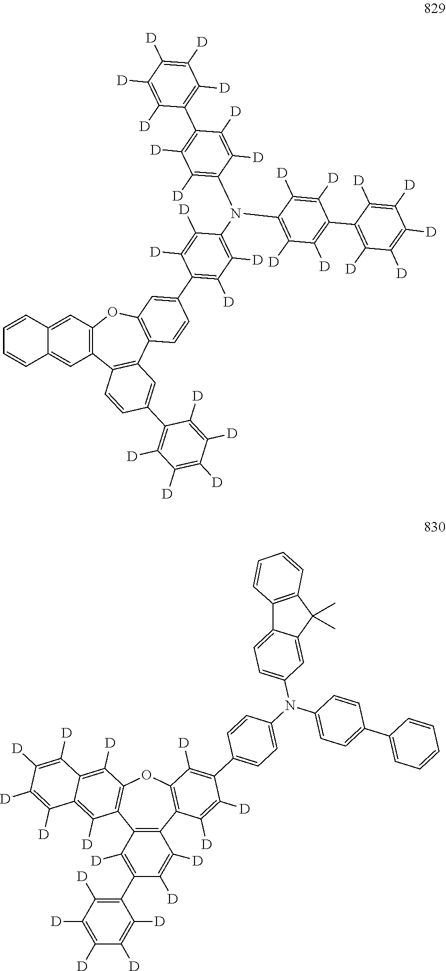 [SMILES: [2H]c1c([2H])c([2H])c(-c2ccc3c(c2)-c2ccc(-c4c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c4[2H])cc2Oc2cc4ccccc4cc2-3)c([2H])c1[2H] ; [2H]c1c([2H])c([2H])c(-c2c([2H])c([2H])c3c(c2[2H])-c2c(c([2H])c4c([2H])c([2H])c([2H])c([2H])c4c2[2H])Oc2c([2H])c(-c4ccc(N(c5ccc(-c6ccccc6)cc5)c5ccc6c(c5)C(C)(C)c5ccccc5-6)cc4)c([2H])c([2H])c2-3)c([2H])c1[2H]]

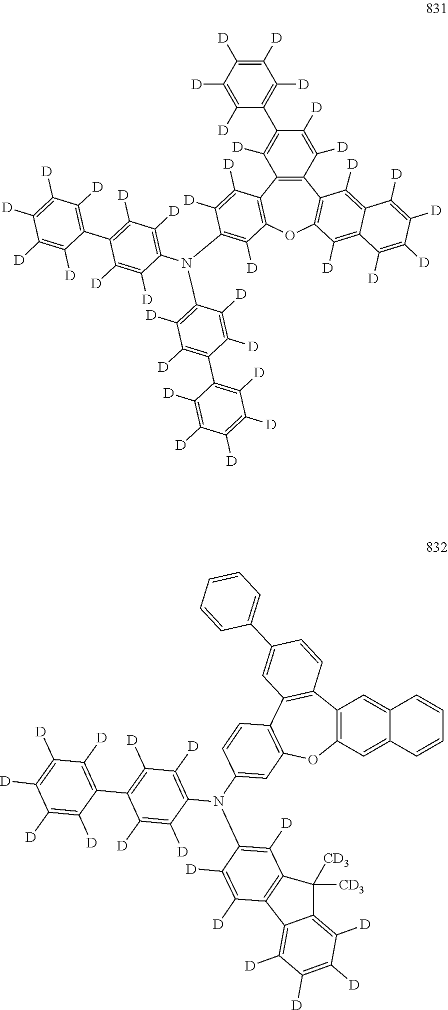

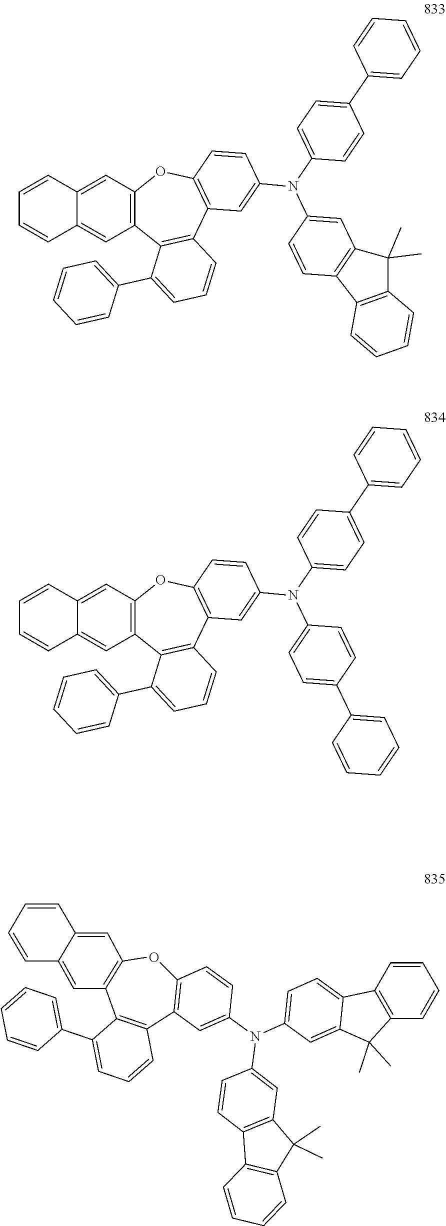 [SMILES: CC1(C)c2ccccc2-c2ccc(N(c3ccc(-c4ccccc4)cc3)c3ccc4c(c3)-c3cccc(-c5ccccc5)c3-c3cc5ccccc5cc3O4)cc21 ; c1ccc(-c2ccc(N(c3ccc(-c4ccccc4)cc3)c3ccc4c(c3)-c3cccc(-c5ccccc5)c3-c3cc5ccccc5cc3O4)cc2)cc1 ; CC1(C)c2ccccc2-c2ccc(N(c3ccc4c(c3)-c3cccc(-c5ccccc5)c3-c3cc5ccccc5cc3O4)c3ccc4c(c3)C(C)(C)c3ccccc3-4)cc21]

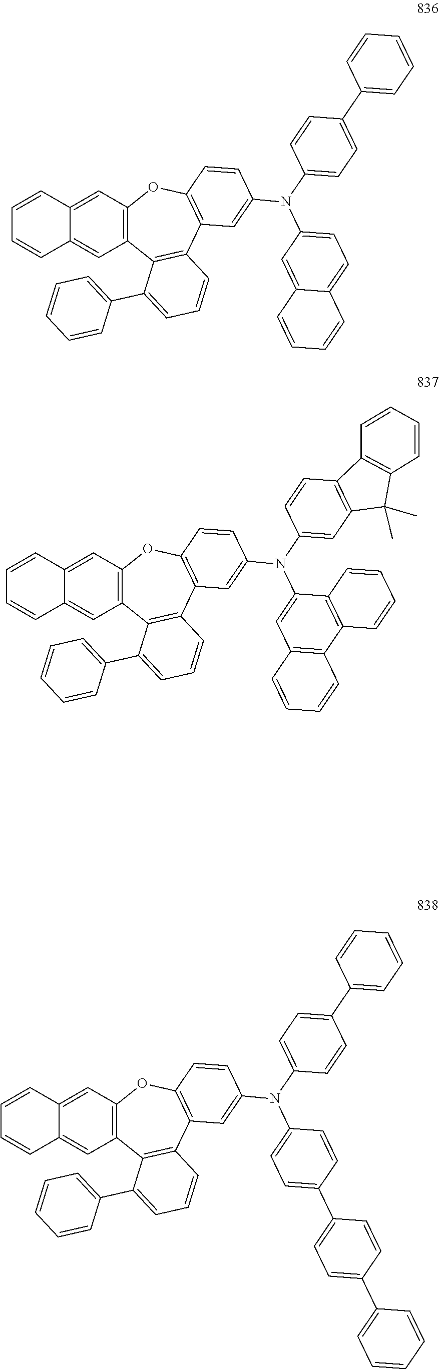 [SMILES: c1ccc(-c2ccc(N(c3ccc4c(c3)-c3cccc(-c5ccccc5)c3-c3cc5ccccc5cc3O4)c3ccc4ccccc4c3)cc2)cc1 ; CC1(C)c2ccccc2-c2ccc(N(c3ccc4c(c3)-c3cccc(-c5ccccc5)c3-c3cc5ccccc5cc3O4)c3cc4ccccc4c4ccccc34)cc21 ; c1ccc(-c2ccc(-c3ccc(N(c4ccc(-c5ccccc5)cc4)c4ccc5c(c4)-c4cccc(-c6ccccc6)c4-c4cc6ccccc6cc4O5)cc3)cc2)cc1]

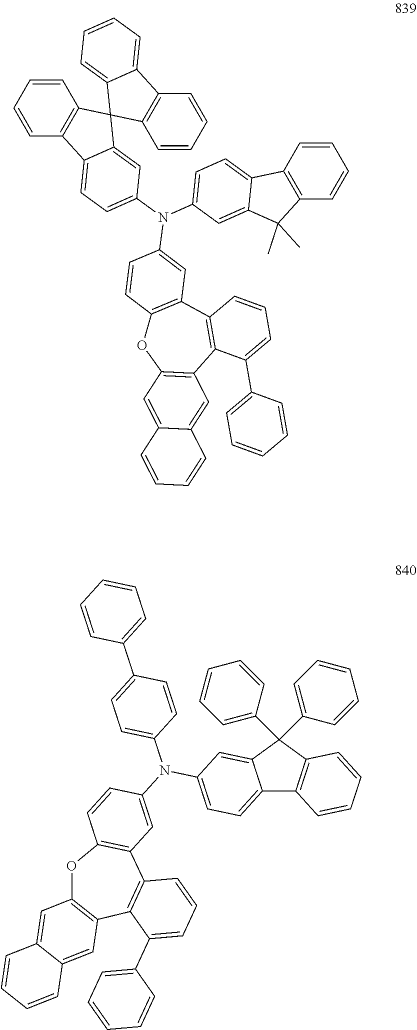 [SMILES: CC1(C)c2ccccc2-c2ccc(N(c3ccc4c(c3)-c3cccc(-c5ccccc5)c3-c3cc5ccccc5cc3O4)c3ccc4c(c3)C3(c5ccccc5-c5ccccc53)c3ccccc3-4)cc21 ; c1ccc(-c2ccc(N(c3ccc4c(c3)-c3cccc(-c5ccccc5)c3-c3cc5ccccc5cc3O4)c3ccc4c(c3)C(c3ccccc3)(c3ccccc3)c3ccccc3-4)cc2)cc1]

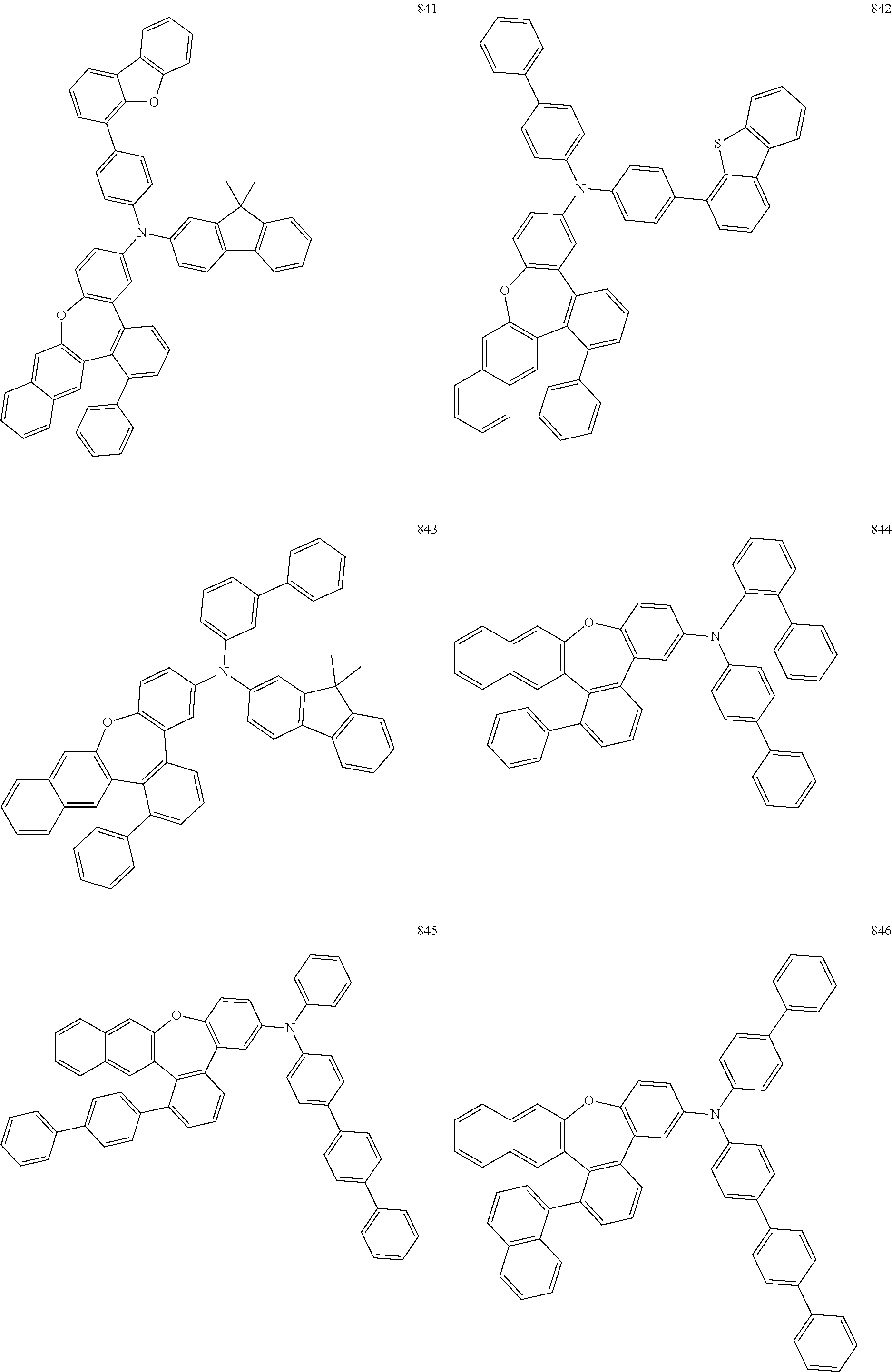

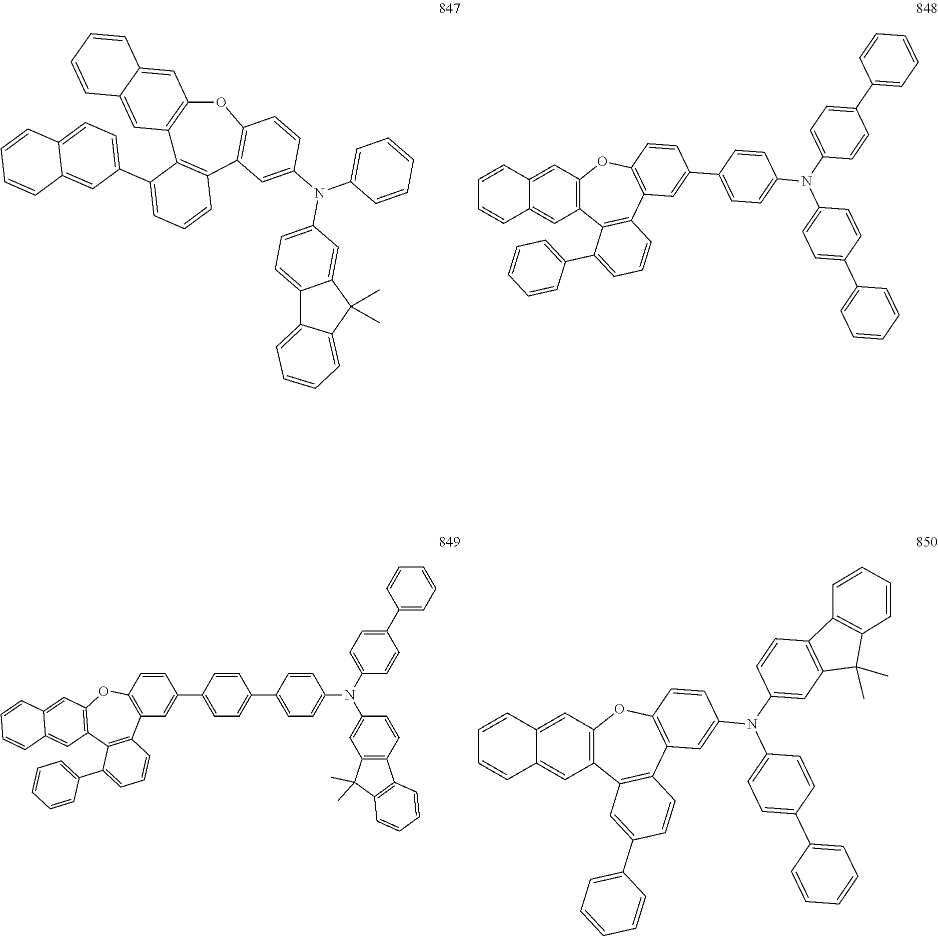 [SMILES: CC1(C)c2ccccc2-c2ccc(N(c3ccccc3)c3ccc4c(c3)-c3cccc(-c5ccc6ccccc6c5)c3-c3cc5ccccc5cc3O4)cc21 ; c1ccc(-c2ccc(N(c3ccc(-c4ccccc4)cc3)c3ccc(-c4ccc5c(c4)-c4cccc(-c6ccccc6)c4-c4cc6ccccc6cc4O5)cc3)cc2)cc1 ; CC1(C)c2ccccc2-c2ccc(N(c3ccc(-c4ccccc4)cc3)c3ccc(-c4ccc(-c5ccc6c(c5)-c5cccc(-c7ccccc7)c5-c5cc7ccccc7cc5O6)cc4)cc3)cc21 ; CC1(C)c2ccccc2-c2ccc(N(c3ccc(-c4ccccc4)cc3)c3ccc4c(c3)-c3ccc(-c5ccccc5)cc3-c3cc5ccccc5cc3O4)cc21]

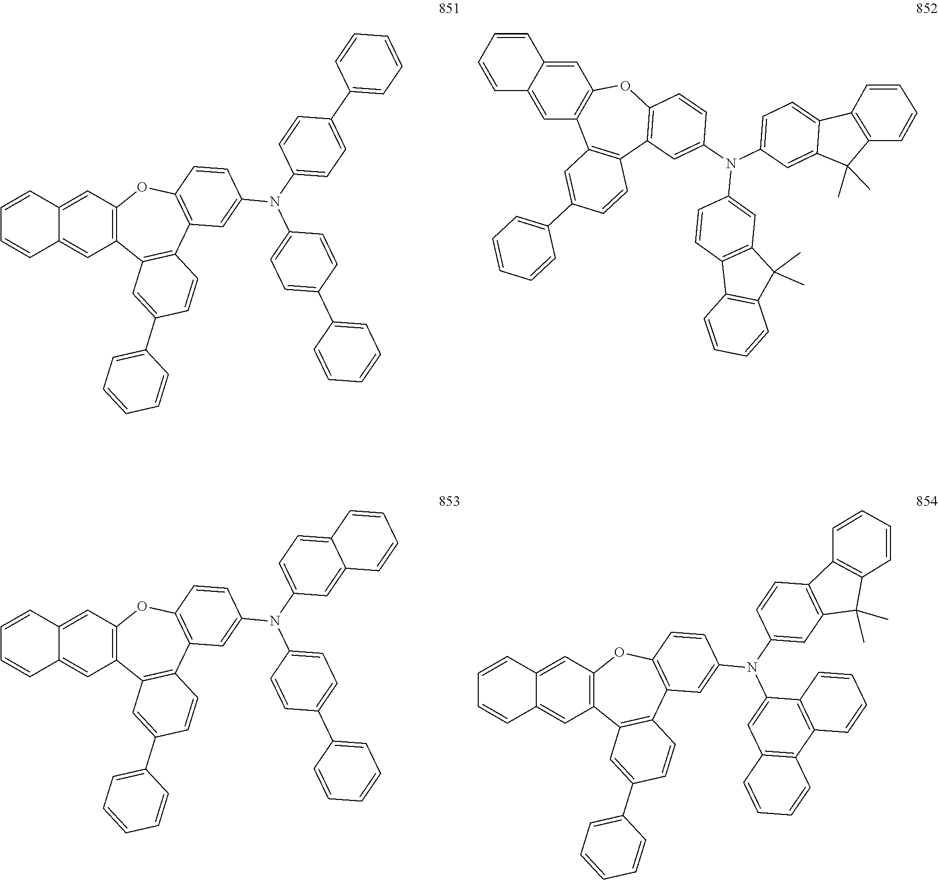 [SMILES: c1ccc(-c2ccc(N(c3ccc(-c4ccccc4)cc3)c3ccc4c(c3)-c3ccc(-c5ccccc5)cc3-c3cc5ccccc5cc3O4)cc2)cc1 ; CC1(C)c2ccccc2-c2ccc(N(c3ccc4c(c3)-c3ccc(-c5ccccc5)cc3-c3cc5ccccc5cc3O4)c3ccc4c(c3)C(C)(C)c3ccccc3-4)cc21 ; c1ccc(-c2ccc(N(c3ccc4c(c3)-c3ccc(-c5ccccc5)cc3-c3cc5ccccc5cc3O4)c3ccc4ccccc4c3)cc2)cc1 ; CC1(C)c2ccccc2-c2ccc(N(c3ccc4c(c3)-c3ccc(-c5ccccc5)cc3-c3cc5ccccc5cc3O4)c3cc4ccccc4c4ccccc34)cc21]

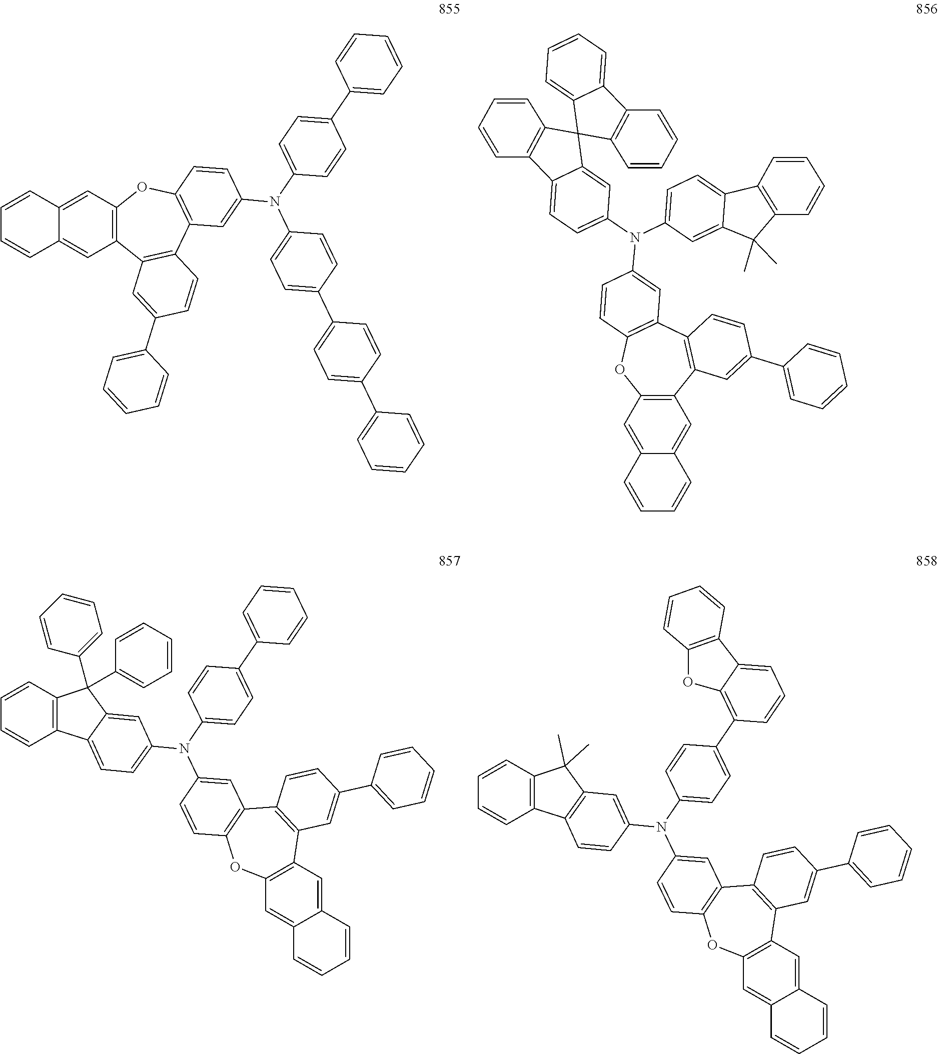

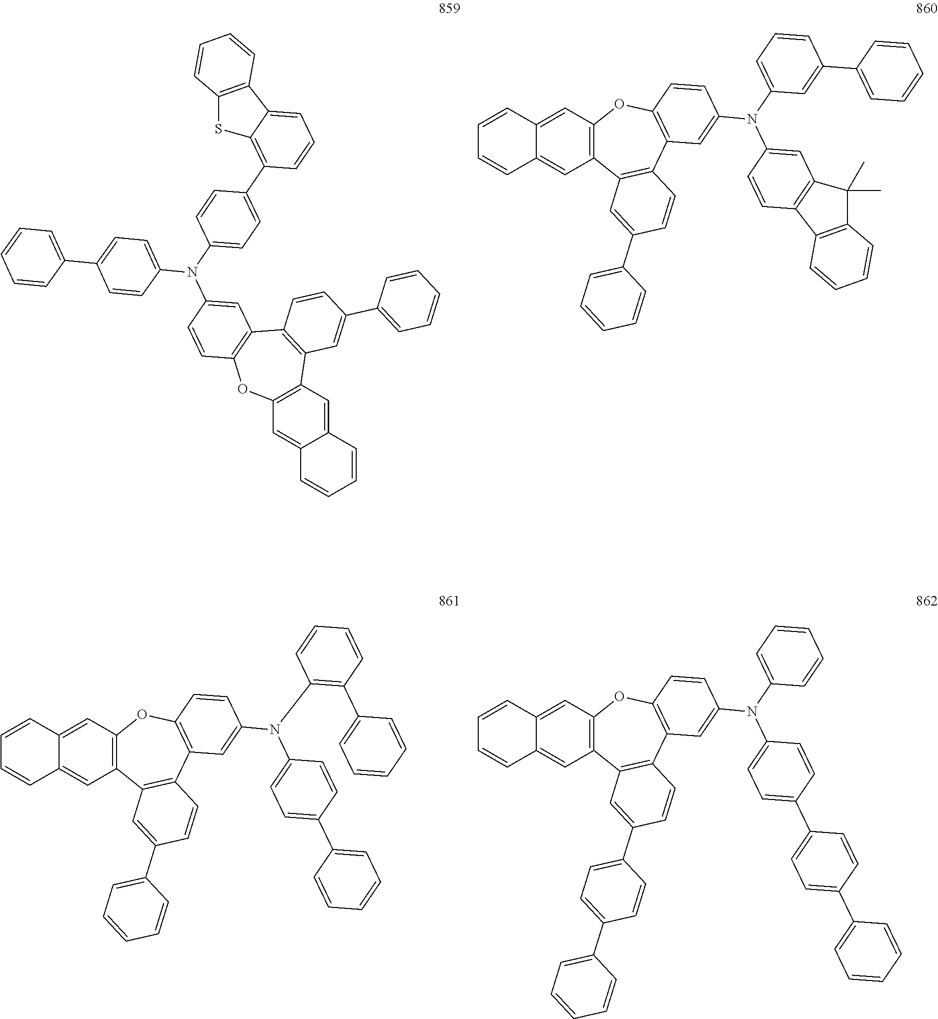 [SMILES: c1ccc(-c2ccc(N(c3ccc(-c4cccc5c4sc4ccccc45)cc3)c3ccc4c(c3)-c3ccc(-c5ccccc5)cc3-c3cc5ccccc5cc3O4)cc2)cc1 ; CC1(C)c2ccccc2-c2ccc(N(c3cccc(-c4ccccc4)c3)c3ccc4c(c3)-c3ccc(-c5ccccc5)cc3-c3cc5ccccc5cc3O4)cc21 ; c1ccc(-c2ccc(N(c3ccc4c(c3)-c3ccc(-c5ccccc5)cc3-c3cc5ccccc5cc3O4)c3ccccc3-c3ccccc3)cc2)cc1 ; c1ccc(-c2ccc(-c3ccc(N(c4ccccc4)c4ccc5c(c4)-c4ccc(-c6ccc(-c7ccccc7)cc6)cc4-c4cc6ccccc6cc4O5)cc3)cc2)cc1]

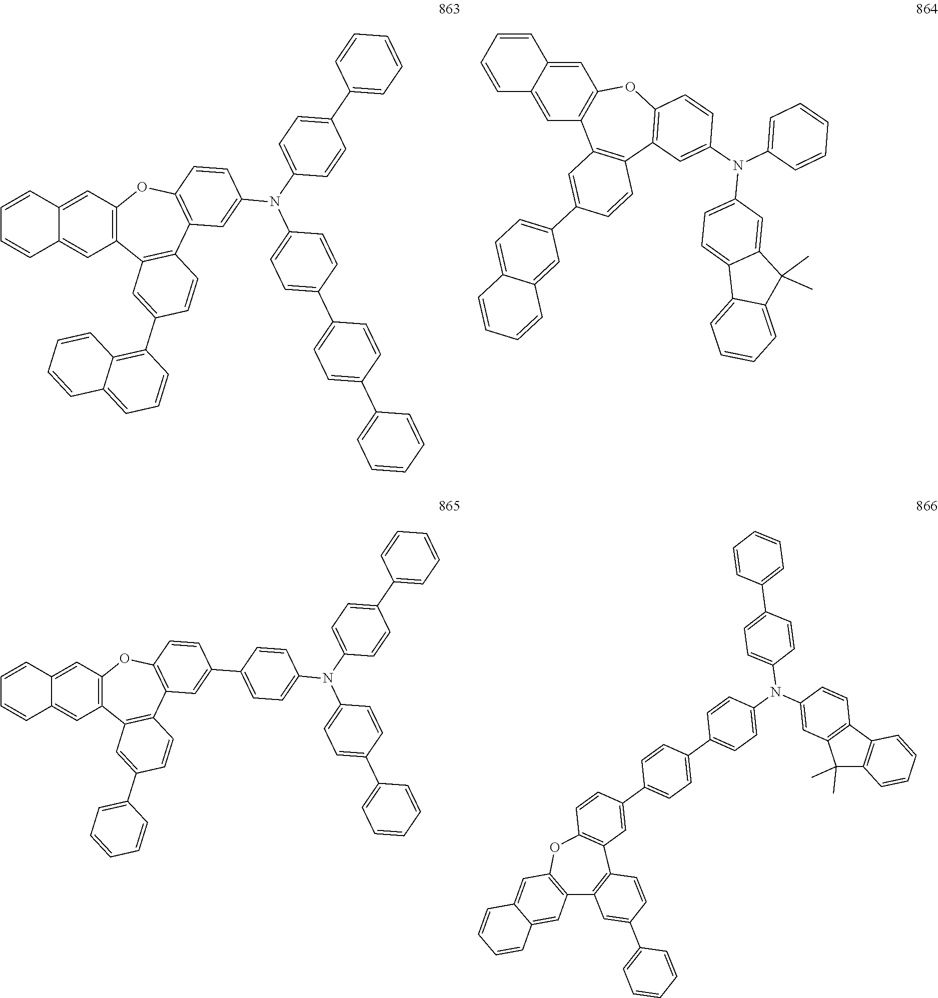 [SMILES: c1ccc(-c2ccc(-c3ccc(N(c4ccc(-c5ccccc5)cc4)c4ccc5c(c4)-c4ccc(-c6cccc7ccccc67)cc4-c4cc6ccccc6cc4O5)cc3)cc2)cc1 ; CC1(C)c2ccccc2-c2ccc(N(c3ccccc3)c3ccc4c(c3)-c3ccc(-c5ccc6ccccc6c5)cc3-c3cc5ccccc5cc3O4)cc21 ; c1ccc(-c2ccc(N(c3ccc(-c4ccccc4)cc3)c3ccc(-c4ccc5c(c4)-c4ccc(-c6ccccc6)cc4-c4cc6ccccc6cc4O5)cc3)cc2)cc1 ; CC1(C)c2ccccc2-c2ccc(N(c3ccc(-c4ccccc4)cc3)c3ccc(-c4ccc(-c5ccc6c(c5)-c5ccc(-c7ccccc7)cc5-c5cc7ccccc7cc5O6)cc4)cc3)cc21]

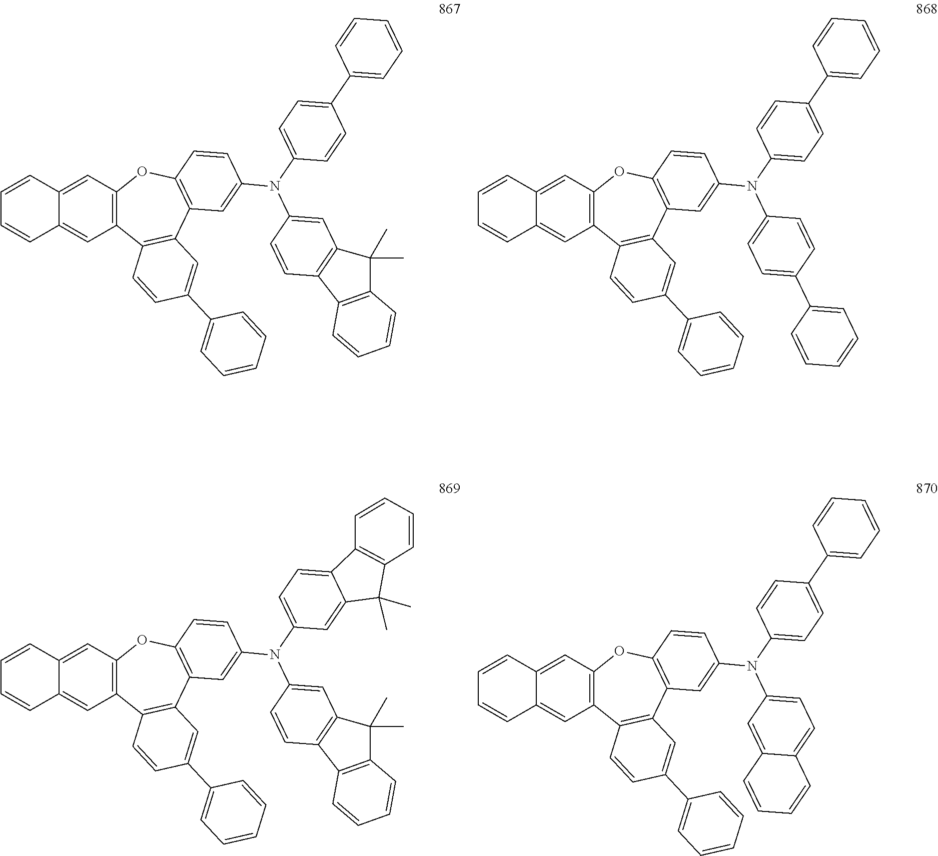 [SMILES: CC1(C)c2ccccc2-c2ccc(N(c3ccc(-c4ccccc4)cc3)c3ccc4c(c3)-c3cc(-c5ccccc5)ccc3-c3cc5ccccc5cc3O4)cc21 ; c1ccc(-c2ccc(N(c3ccc(-c4ccccc4)cc3)c3ccc4c(c3)-c3cc(-c5ccccc5)ccc3-c3cc5ccccc5cc3O4)cc2)cc1 ; CC1(C)c2ccccc2-c2ccc(N(c3ccc4c(c3)-c3cc(-c5ccccc5)ccc3-c3cc5ccccc5cc3O4)c3ccc4c(c3)C(C)(C)c3ccccc3-4)cc21 ; c1ccc(-c2ccc(N(c3ccc4c(c3)-c3cc(-c5ccccc5)ccc3-c3cc5ccccc5cc3O4)c3ccc4ccccc4c3)cc2)cc1]

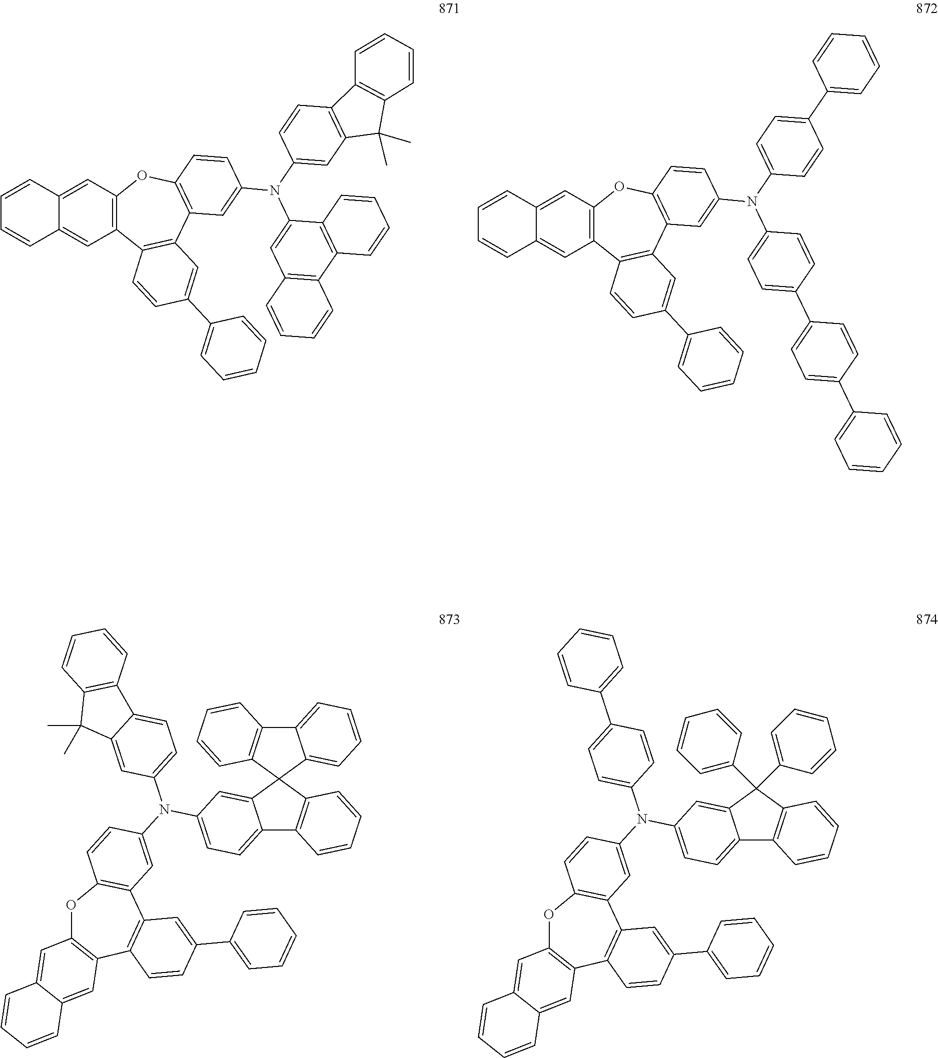 [SMILES: CC1(C)c2ccccc2-c2ccc(N(c3ccc4c(c3)-c3cc(-c5ccccc5)ccc3-c3cc5ccccc5cc3O4)c3cc4ccccc4c4ccccc34)cc21 ; c1ccc(-c2ccc(-c3ccc(N(c4ccc(-c5ccccc5)cc4)c4ccc5c(c4)-c4cc(-c6ccccc6)ccc4-c4cc6ccccc6cc4O5)cc3)cc2)cc1 ; CC1(C)c2ccccc2-c2ccc(N(c3ccc4c(c3)-c3cc(-c5ccccc5)ccc3-c3cc5ccccc5cc3O4)c3ccc4c(c3)C3(c5ccccc5-c5ccccc53)c3ccccc3-4)cc21 ; c1ccc(-c2ccc(N(c3ccc4c(c3)-c3cc(-c5ccccc5)ccc3-c3cc5ccccc5cc3O4)c3ccc4c(c3)C(c3ccccc3)(c3ccccc3)c3ccccc3-4)cc2)cc1]

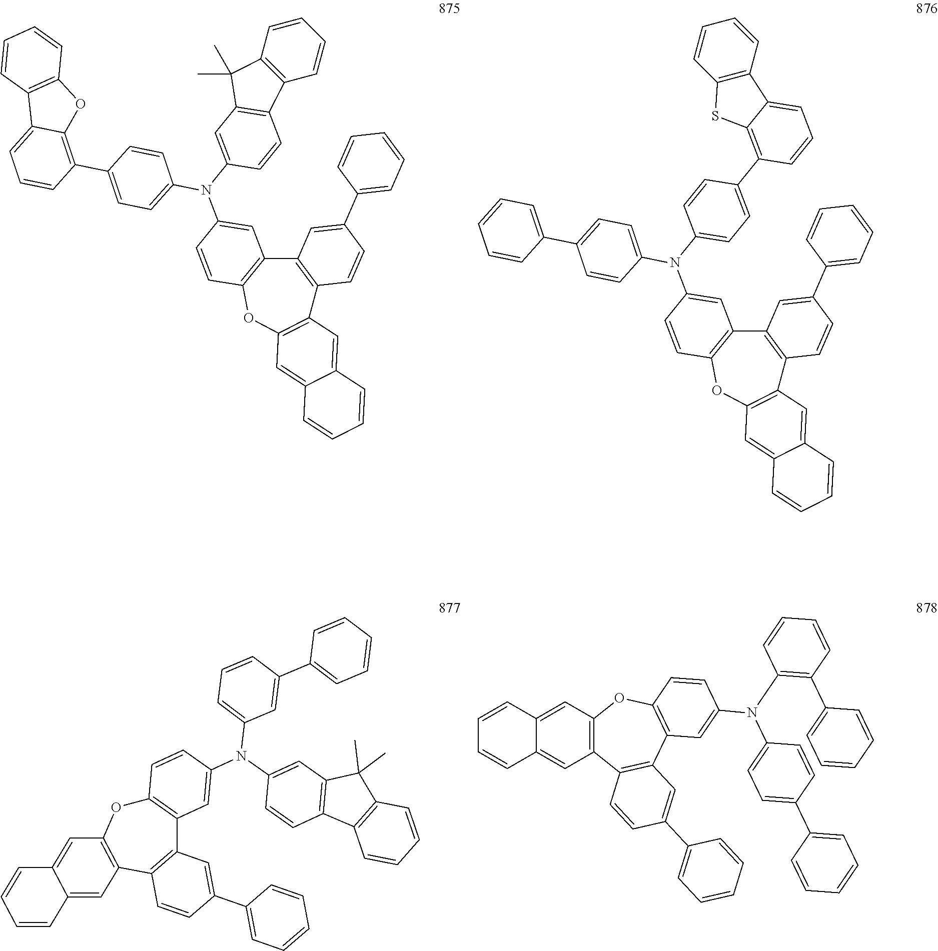 [SMILES: CC1(C)c2ccccc2-c2ccc(N(c3ccc(-c4cccc5c4oc4ccccc45)cc3)c3ccc4c(c3)-c3cc(-c5ccccc5)ccc3-c3cc5ccccc5cc3O4)cc21 ; c1ccc(-c2ccc(N(c3ccc(-c4cccc5c4sc4ccccc45)cc3)c3ccc4c(c3)-c3cc(-c5ccccc5)ccc3-c3cc5ccccc5cc3O4)cc2)cc1 ; CC1(C)c2ccccc2-c2ccc(N(c3cccc(-c4ccccc4)c3)c3ccc4c(c3)-c3cc(-c5ccccc5)ccc3-c3cc5ccccc5cc3O4)cc21 ; c1ccc(-c2ccc(N(c3ccc4c(c3)-c3cc(-c5ccccc5)ccc3-c3cc5ccccc5cc3O4)c3ccccc3-c3ccccc3)cc2)cc1]

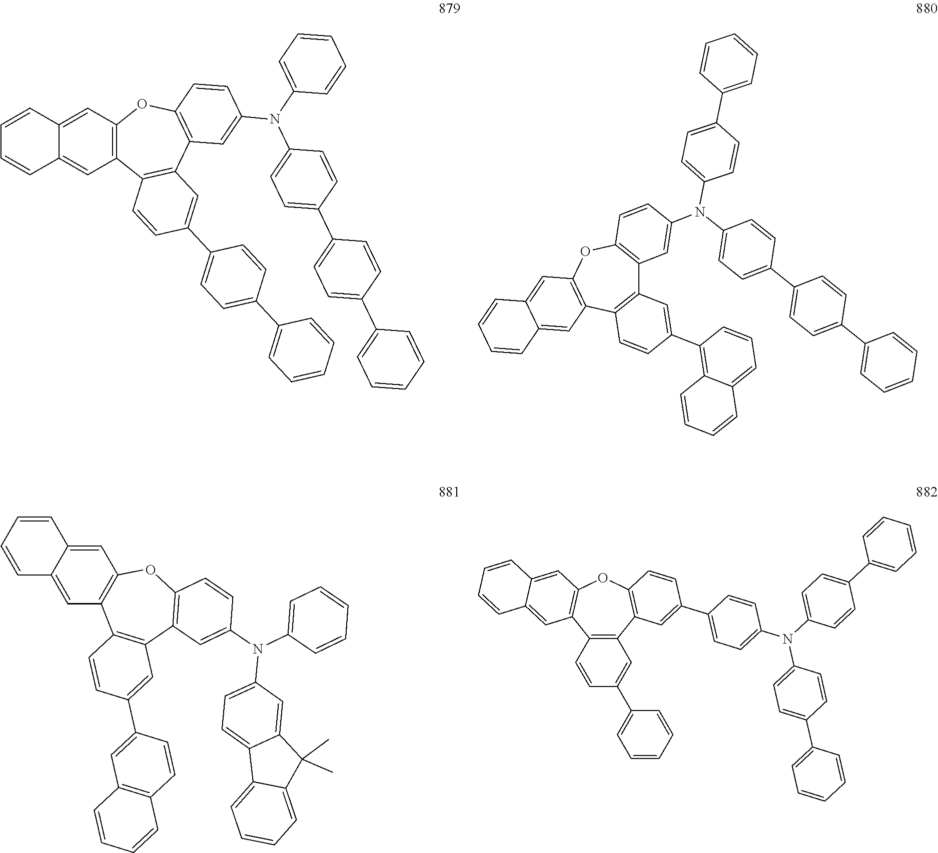 [SMILES: c1ccc(-c2ccc(-c3ccc(N(c4ccccc4)c4ccc5c(c4)-c4cc(-c6ccc(-c7ccccc7)cc6)ccc4-c4cc6ccccc6cc4O5)cc3)cc2)cc1 ; c1ccc(-c2ccc(-c3ccc(N(c4ccc(-c5ccccc5)cc4)c4ccc5c(c4)-c4cc(-c6cccc7ccccc67)ccc4-c4cc6ccccc6cc4O5)cc3)cc2)cc1 ; CC1(C)c2ccccc2-c2ccc(N(c3ccccc3)c3ccc4c(c3)-c3cc(-c5ccc6ccccc6c5)ccc3-c3cc5ccccc5cc3O4)cc21 ; c1ccc(-c2ccc(N(c3ccc(-c4ccccc4)cc3)c3ccc(-c4ccc5c(c4)-c4cc(-c6ccccc6)ccc4-c4cc6ccccc6cc4O5)cc3)cc2)cc1]

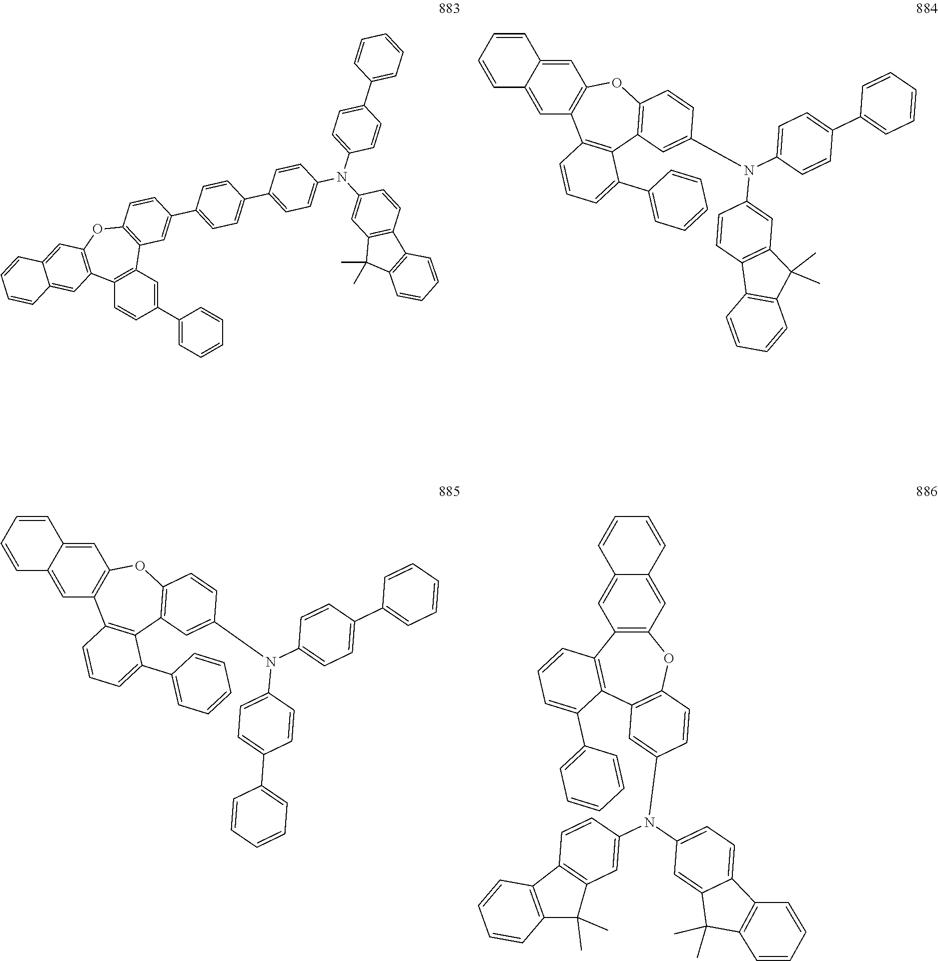 [SMILES: CC1(C)c2ccccc2-c2ccc(N(c3ccc(-c4ccccc4)cc3)c3ccc(-c4ccc(-c5ccc6c(c5)-c5cc(-c7ccccc7)ccc5-c5cc7ccccc7cc5O6)cc4)cc3)cc21 ; CC1(C)c2ccccc2-c2ccc(N(c3ccc(-c4ccccc4)cc3)c3ccc4c(c3)-c3c(-c5ccccc5)cccc3-c3cc5ccccc5cc3O4)cc21 ; c1ccc(-c2ccc(N(c3ccc(-c4ccccc4)cc3)c3ccc4c(c3)-c3c(-c5ccccc5)cccc3-c3cc5ccccc5cc3O4)cc2)cc1 ; CC1(C)c2ccccc2-c2ccc(N(c3ccc4c(c3)-c3c(-c5ccccc5)cccc3-c3cc5ccccc5cc3O4)c3ccc4c(c3)C(C)(C)c3ccccc3-4)cc21]

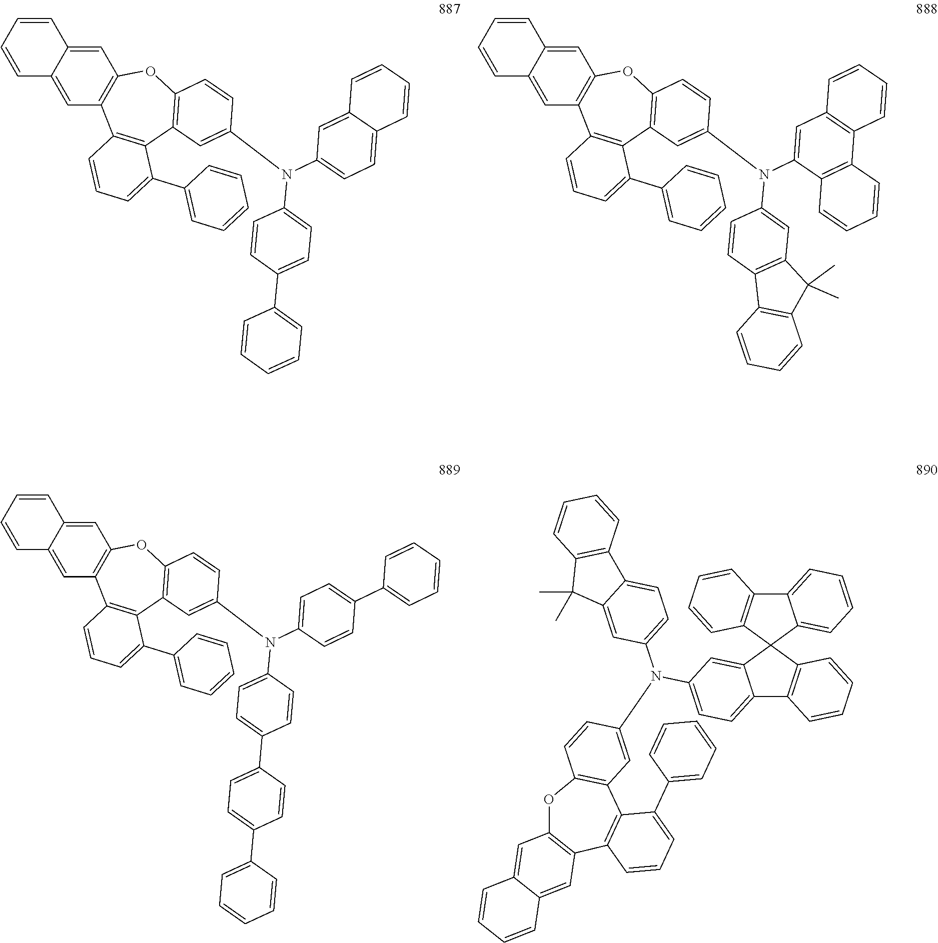 [SMILES: c1ccc(-c2ccc(N(c3ccc4c(c3)-c3c(-c5ccccc5)cccc3-c3cc5ccccc5cc3O4)c3ccc4ccccc4c3)cc2)cc1 ; CC1(C)c2ccccc2-c2ccc(N(c3ccc4c(c3)-c3c(-c5ccccc5)cccc3-c3cc5ccccc5cc3O4)c3cc4ccccc4c4ccccc34)cc21 ; c1ccc(-c2ccc(-c3ccc(N(c4ccc(-c5ccccc5)cc4)c4ccc5c(c4)-c4c(-c6ccccc6)cccc4-c4cc6ccccc6cc4O5)cc3)cc2)cc1 ; CC1(C)c2ccccc2-c2ccc(N(c3ccc4c(c3)-c3c(-c5ccccc5)cccc3-c3cc5ccccc5cc3O4)c3ccc4c(c3)C3(c5ccccc5-c5ccccc53)c3ccccc3-4)cc21]

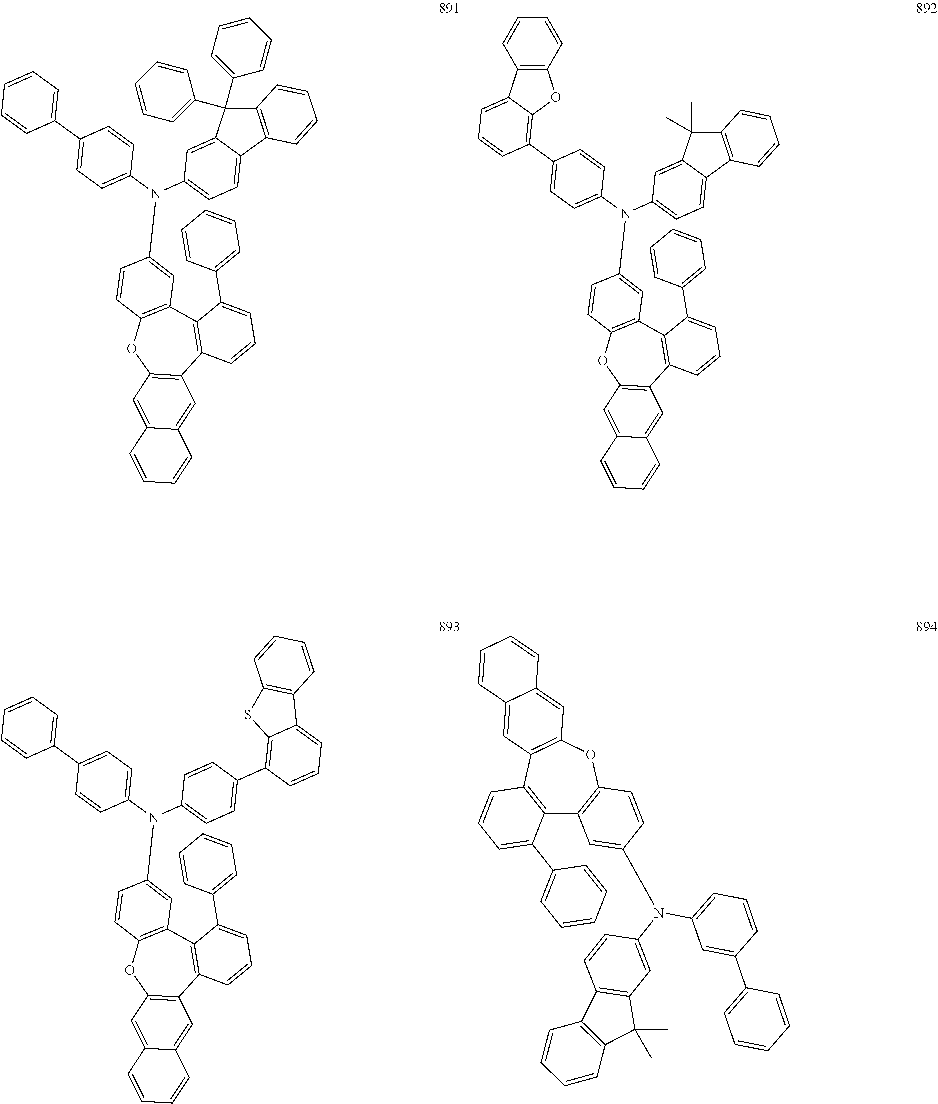 [SMILES: c1ccc(-c2ccc(N(c3ccc4c(c3)-c3c(-c5ccccc5)cccc3-c3cc5ccccc5cc3O4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)-c3c(-c5ccccc5)cccc3-c3cc5ccccc5cc3O4)cc21 ; c1ccc(-c2ccc(N(c3ccc(-c4cccc5c4sc4ccccc45)cc3)c3ccc4c(c3)-c3c(-c5ccccc5)cccc3-c3cc5ccccc5cc3O4)cc2)cc1 ; CC1(C)c2ccccc2-c2ccc(N(c3cccc(-c4ccccc4)c3)c3ccc4c(c3)-c3c(-c5ccccc5)cccc3-c3cc5ccccc5cc3O4)cc21]

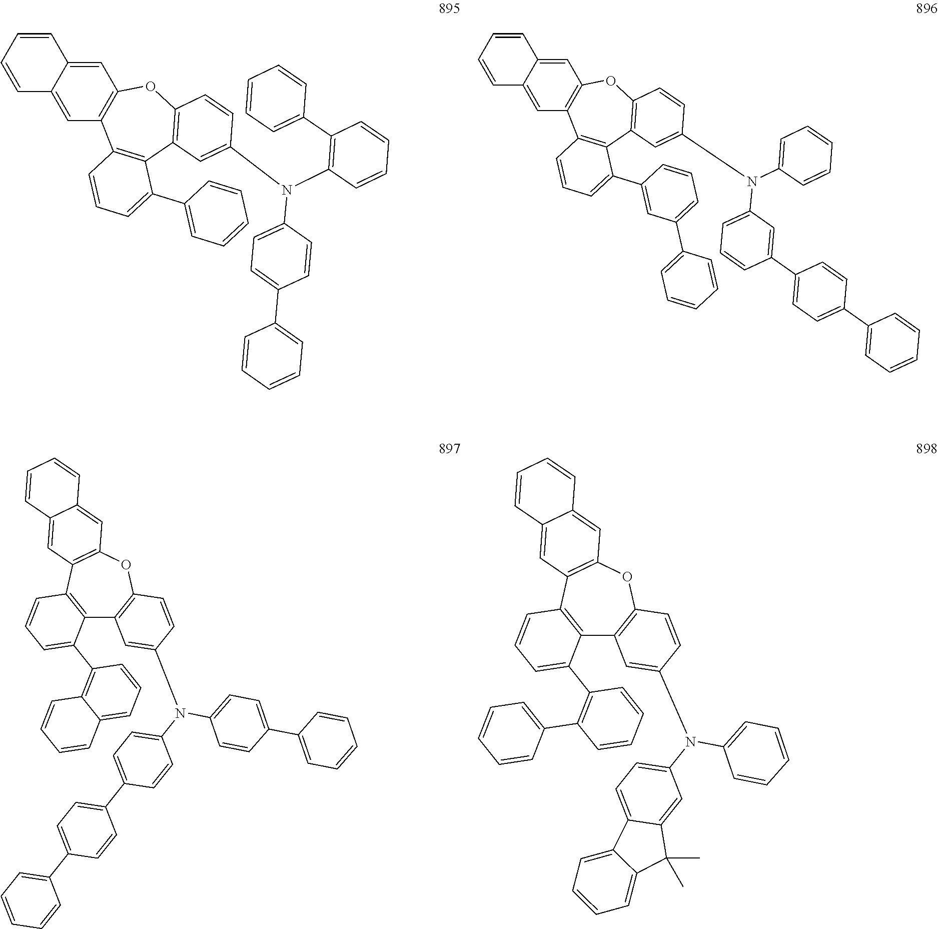 [SMILES: c1ccc(-c2ccc(N(c3ccc4c(c3)-c3c(-c5ccccc5)cccc3-c3cc5ccccc5cc3O4)c3ccccc3-c3ccccc3)cc2)cc1 ; c1ccc(-c2ccc(-c3cccc(N(c4ccccc4)c4ccc5c(c4)-c4c(-c6cccc(-c7ccccc7)c6)cccc4-c4cc6ccccc6cc4O5)c3)cc2)cc1 ; c1ccc(-c2ccc(-c3ccc(N(c4ccc(-c5ccccc5)cc4)c4ccc5c(c4)-c4c(cccc4-c4cccc6ccccc46)-c4cc6ccccc6cc4O5)cc3)cc2)cc1 ; CC1(C)c2ccccc2-c2ccc(N(c3ccccc3)c3ccc4c(c3)-c3c(cccc3-c3ccccc3-c3ccccc3)-c3cc5ccccc5cc3O4)cc21]

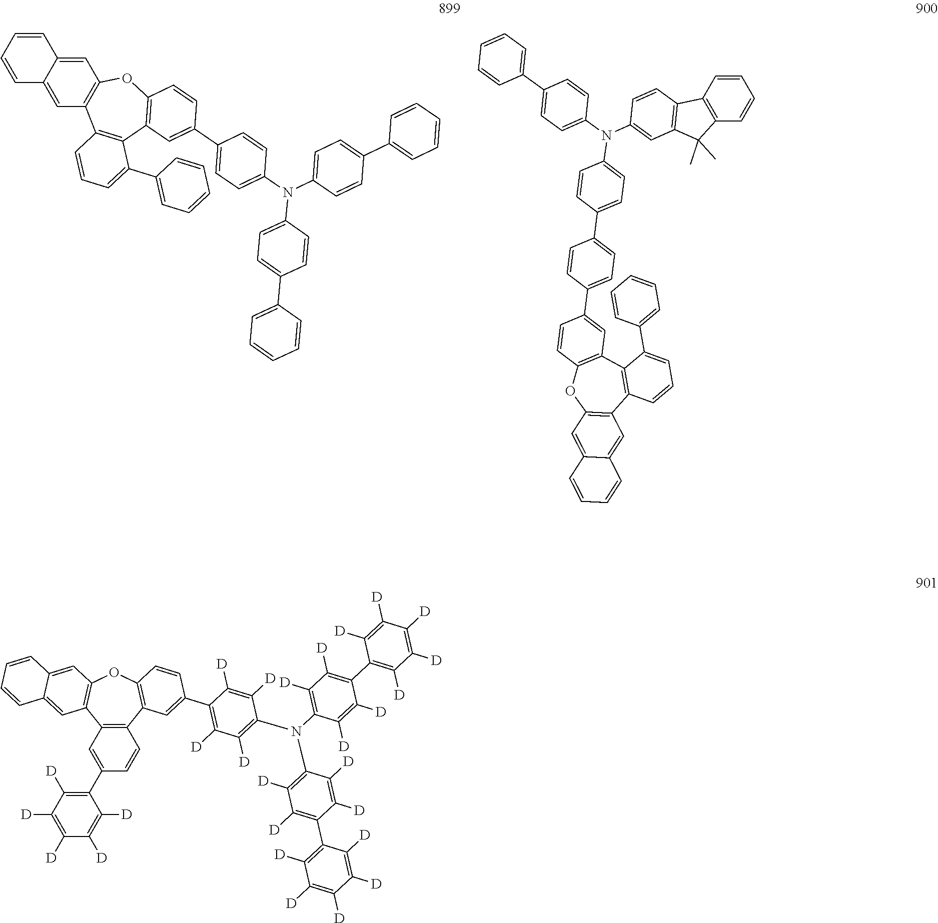 [SMILES: c1ccc(-c2ccc(N(c3ccc(-c4ccccc4)cc3)c3ccc(-c4ccc5c(c4)-c4c(-c6ccccc6)cccc4-c4cc6ccccc6cc4O5)cc3)cc2)cc1 ; CC1(C)c2ccccc2-c2ccc(N(c3ccc(-c4ccccc4)cc3)c3ccc(-c4ccc(-c5ccc6c(c5)-c5c(-c7ccccc7)cccc5-c5cc7ccccc7cc5O6)cc4)cc3)cc21 ; [2H]c1c([2H])c([2H])c(-c2ccc3c(c2)-c2cc4ccccc4cc2Oc2ccc(-c4c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c4[2H])cc2-3)c([2H])c1[2H]]

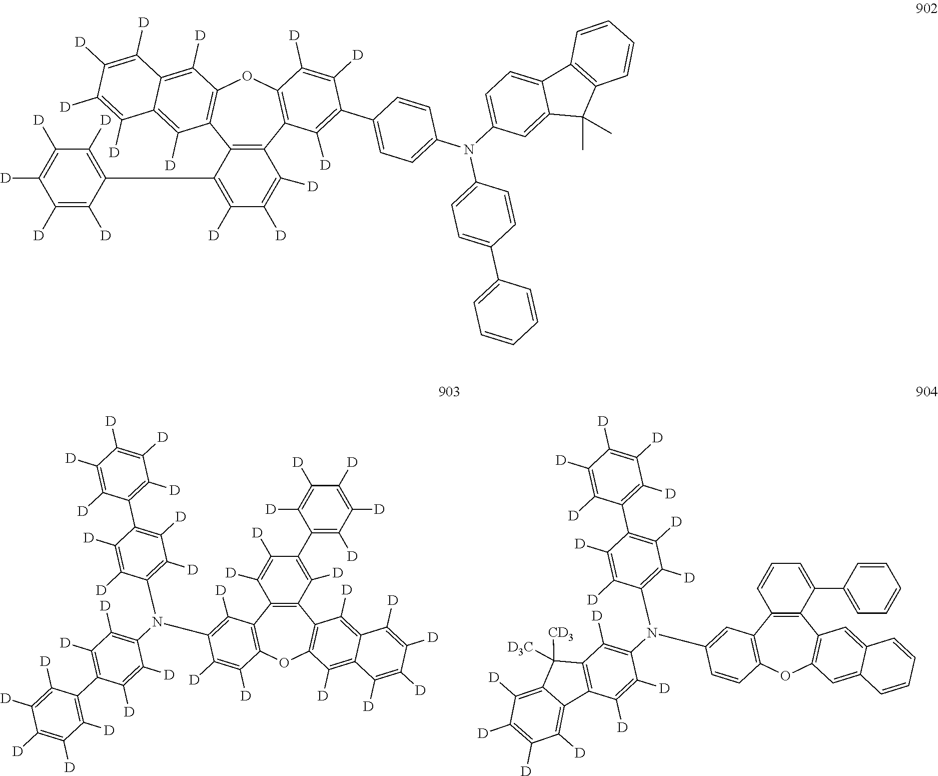

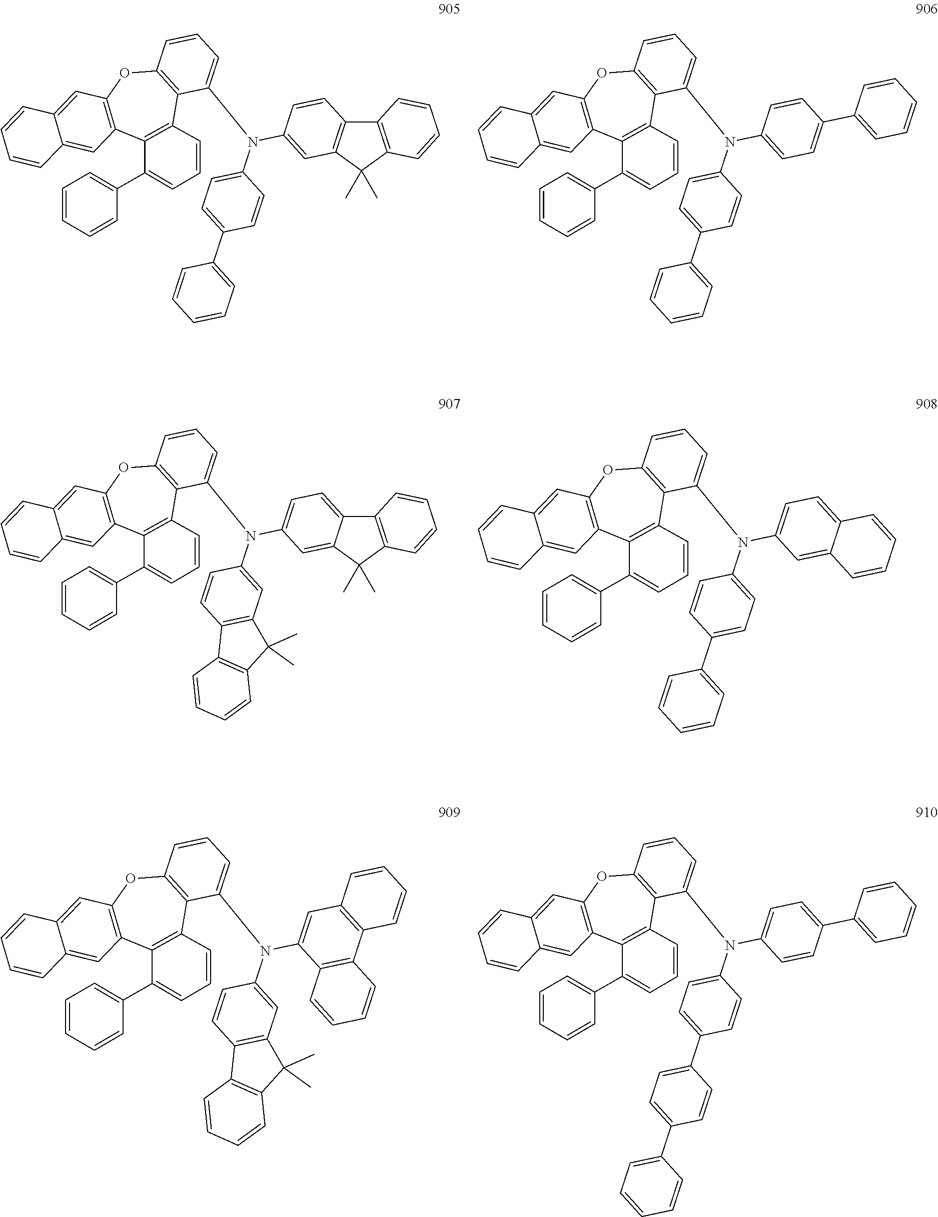

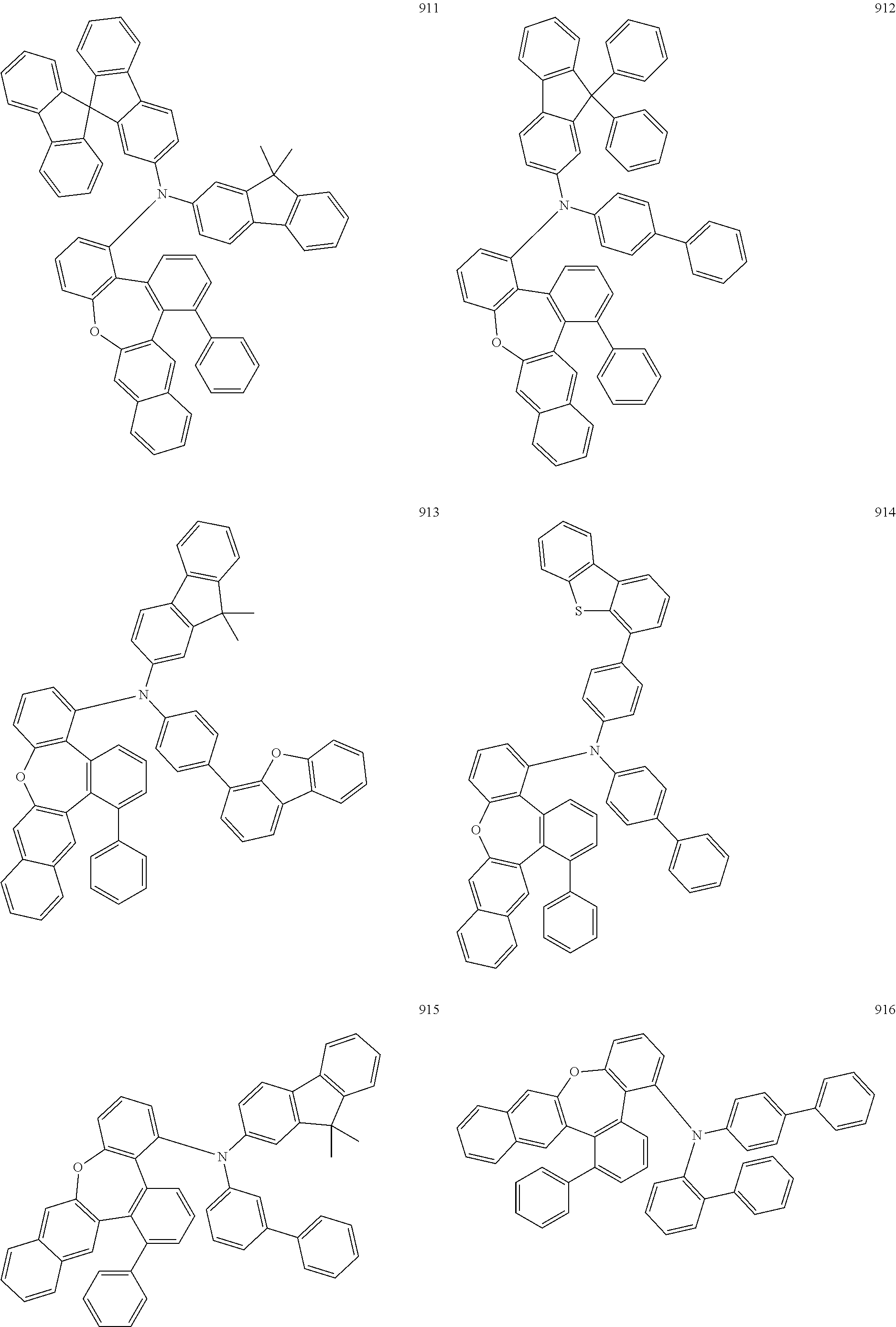

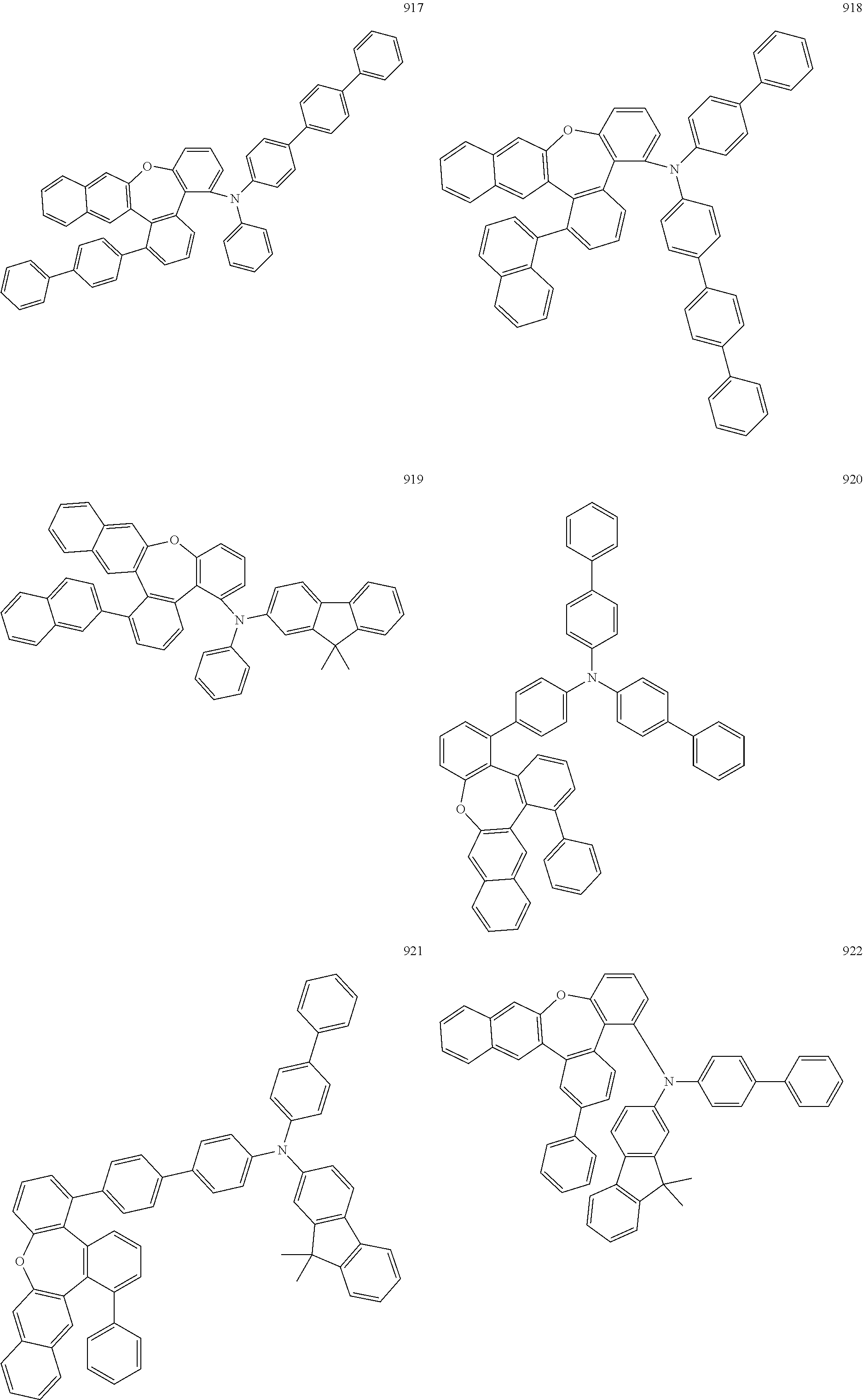

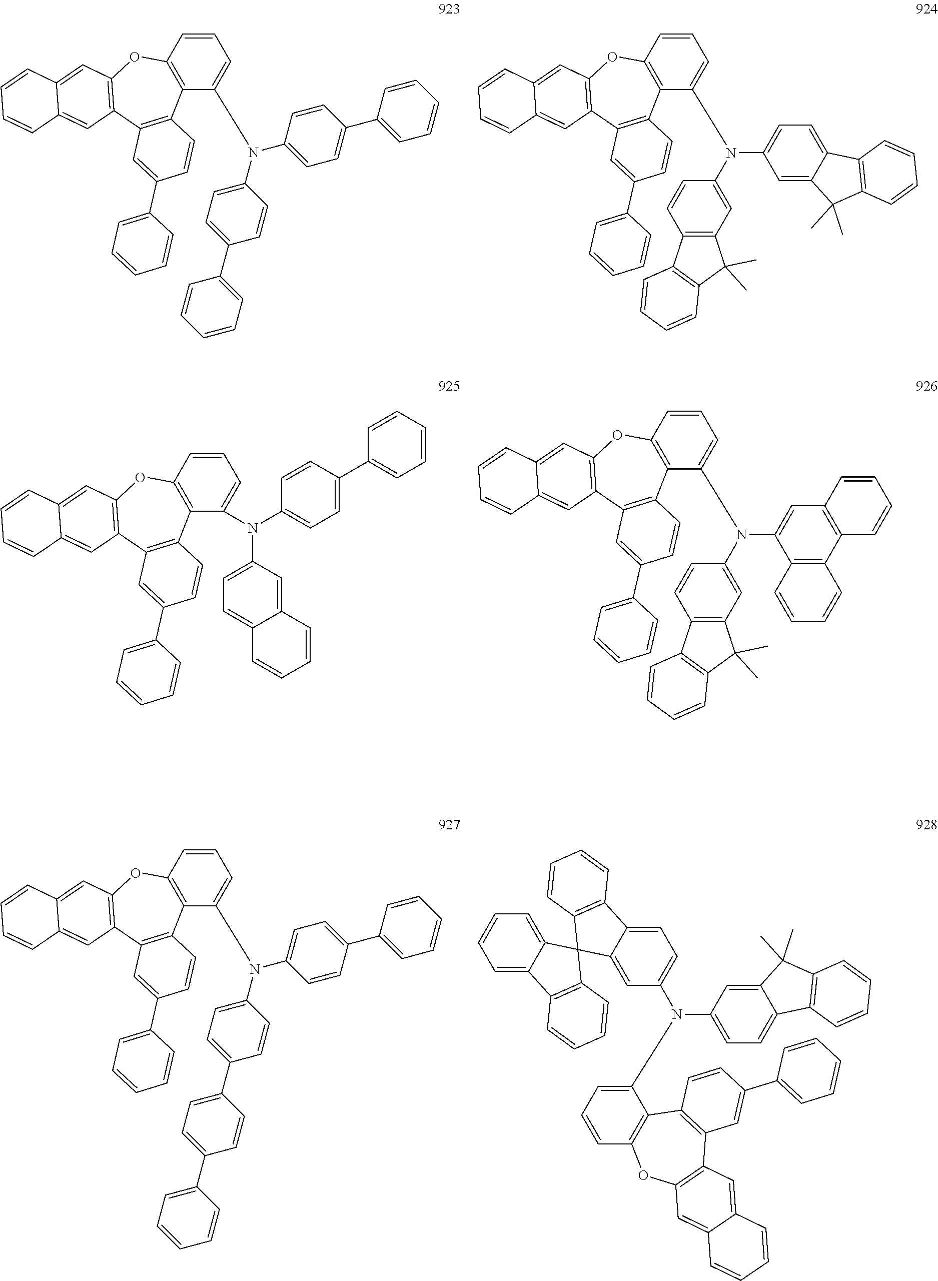

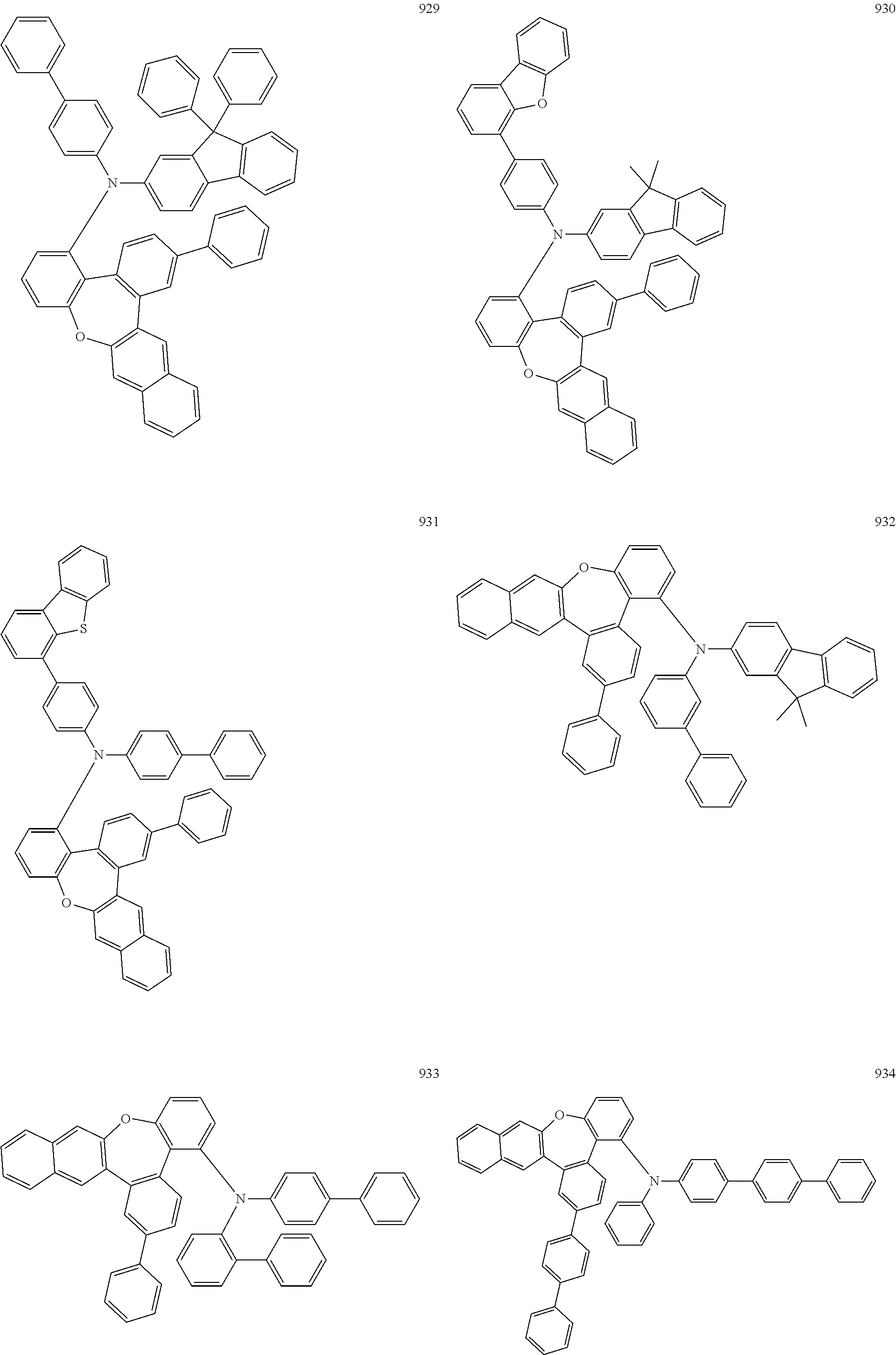

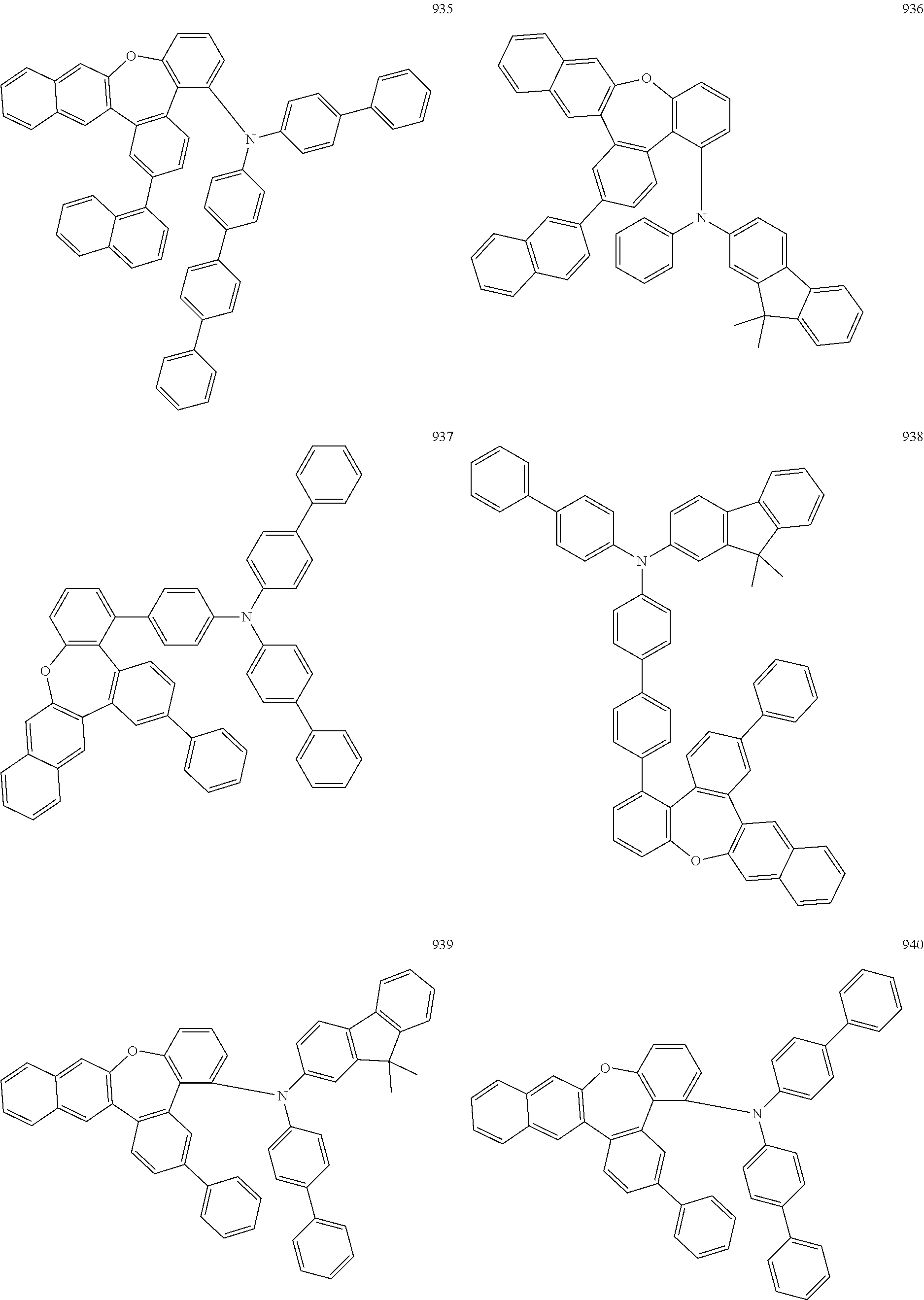

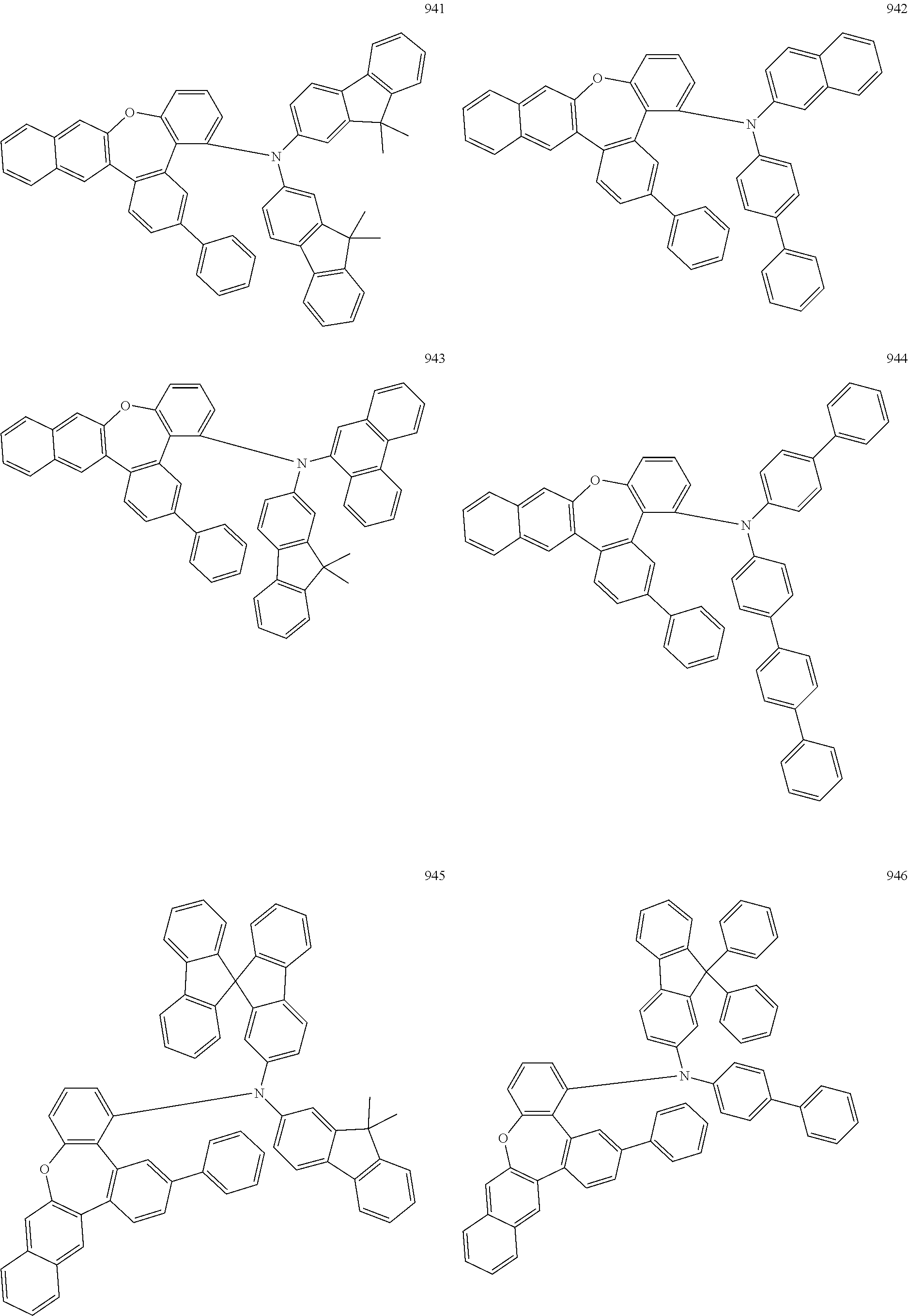

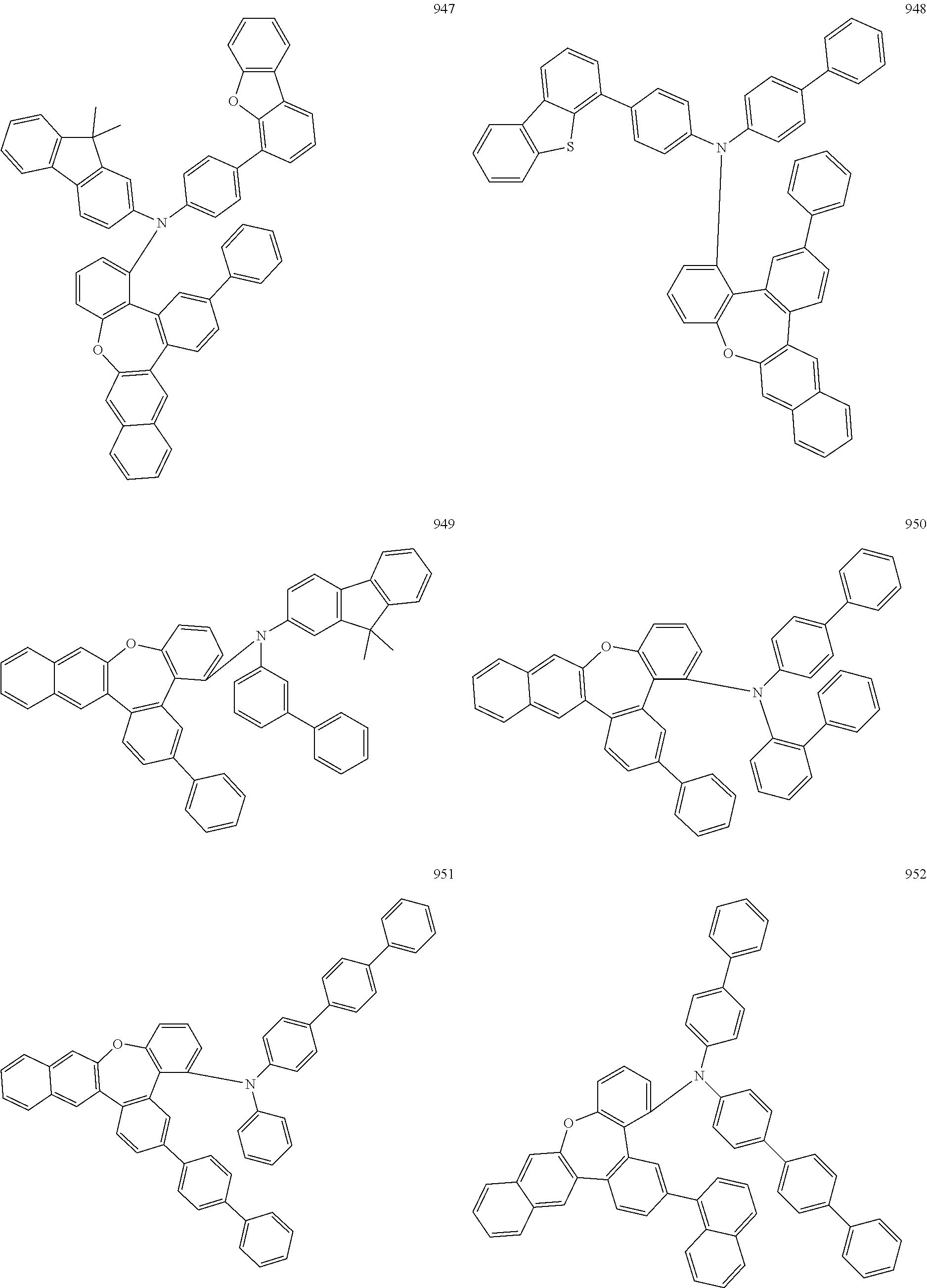

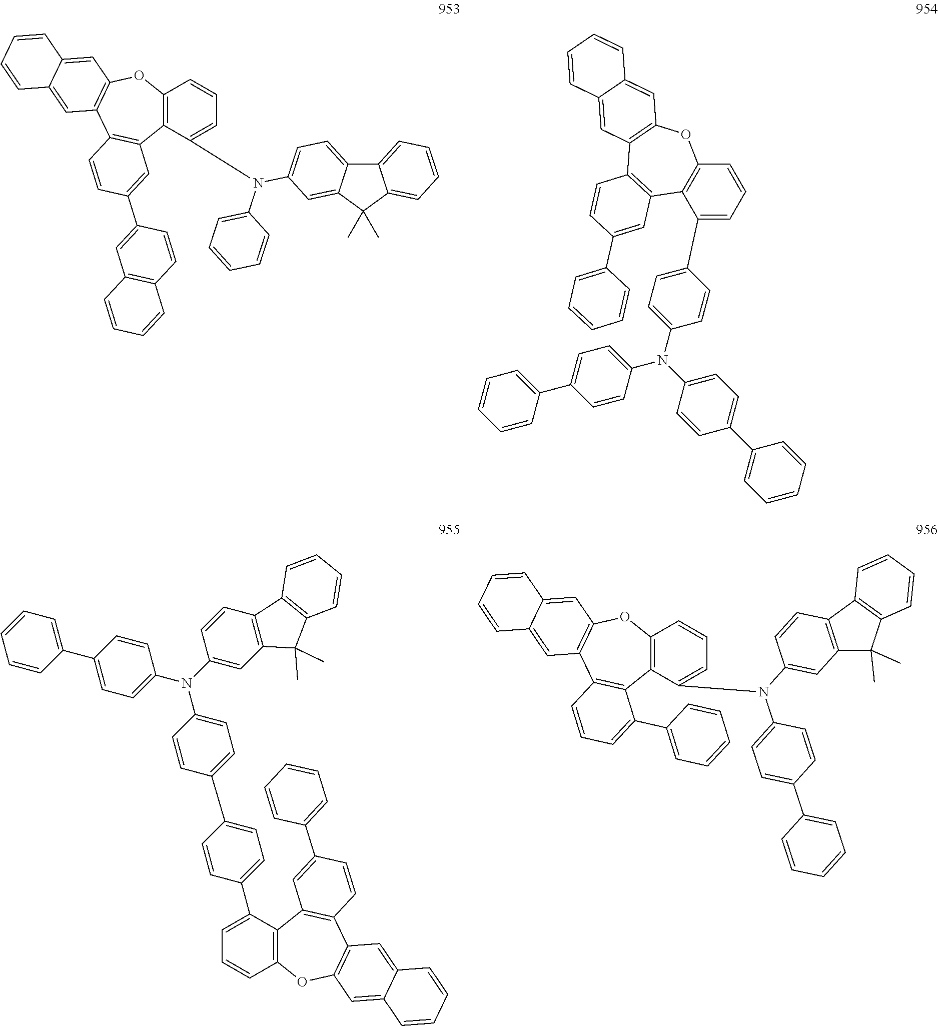 [SMILES: CC1(C)c2ccccc2-c2ccc(N(c3ccccc3)c3cccc4c3-c3cc(-c5ccc6ccccc6c5)ccc3-c3cc5ccccc5cc3O4)cc21 ; c1ccc(-c2ccc(N(c3ccc(-c4ccccc4)cc3)c3ccc(-c4cccc5c4-c4cc(-c6ccccc6)ccc4-c4cc6ccccc6cc4O5)cc3)cc2)cc1 ; CC1(C)c2ccccc2-c2ccc(N(c3ccc(-c4ccccc4)cc3)c3ccc(-c4ccc(-c5cccc6c5-c5cc(-c7ccccc7)ccc5-c5cc7ccccc7cc5O6)cc4)cc3)cc21 ; CC1(C)c2ccccc2-c2ccc(N(c3ccc(-c4ccccc4)cc3)c3cccc4c3-c3c(-c5ccccc5)cccc3-c3cc5ccccc5cc3O4)cc21]

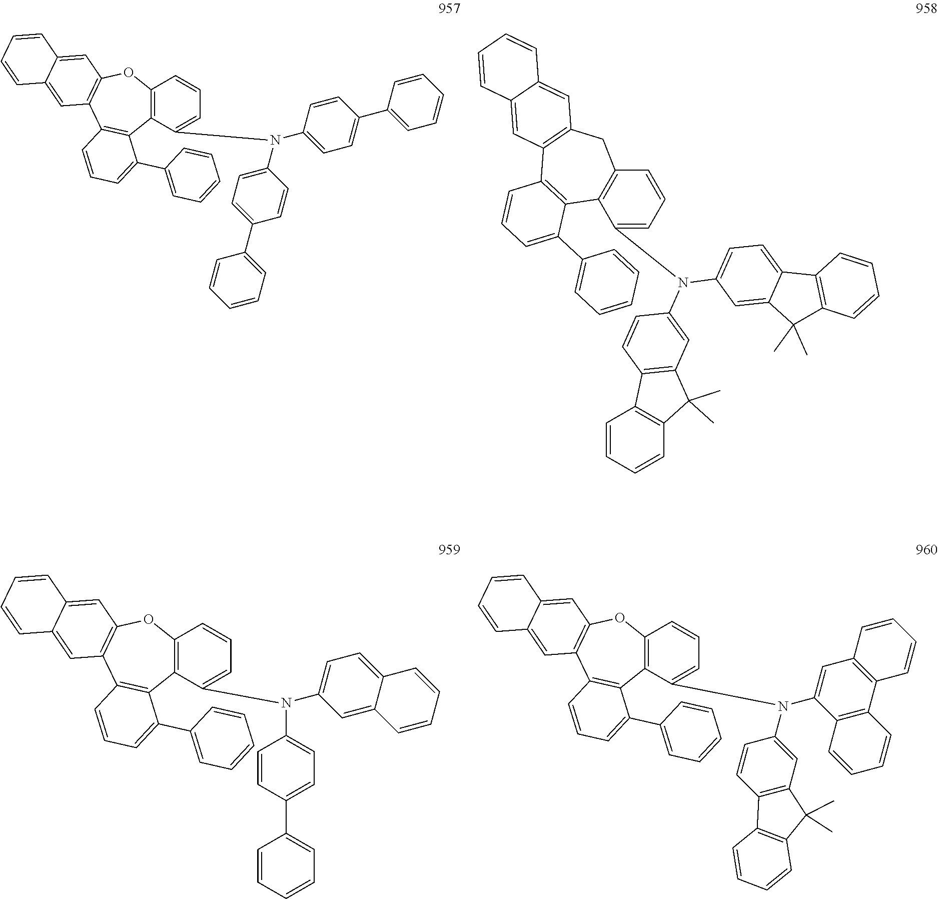

[chemical structure]

[chemical structure]

[chemical structure]

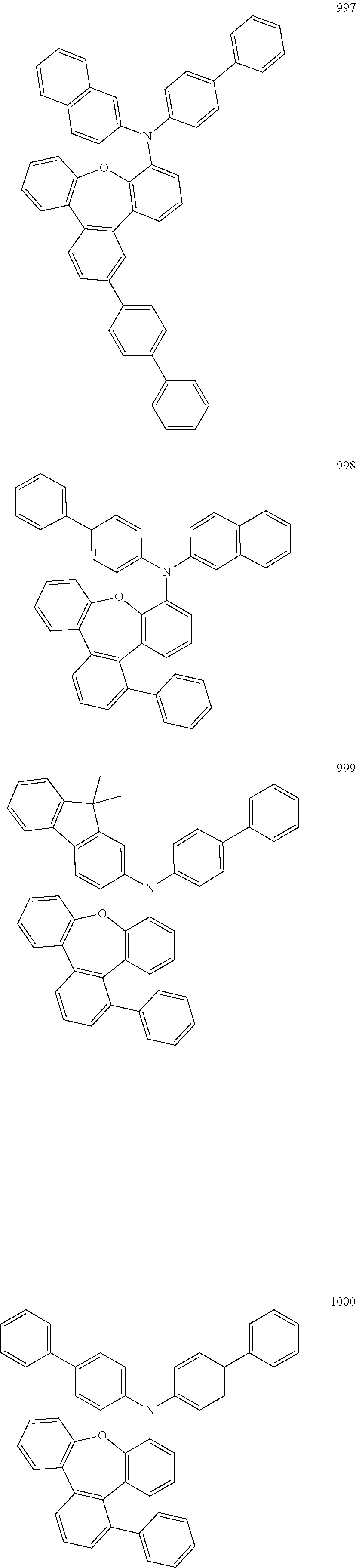 [SMILES: c1ccc(-c2ccc(-c3ccc4c(c3)-c3cccc(N(c5ccc(-c6ccccc6)cc5)c5ccc6ccccc6c5)c3Oc3ccccc3-4)cc2)cc1 ; c1ccc(-c2ccc(N(c3ccc4ccccc4c3)c3cccc4c3Oc3ccccc3-c3cccc(-c5ccccc5)c3-4)cc2)cc1 ; CC1(C)c2ccccc2-c2ccc(N(c3ccc(-c4ccccc4)cc3)c3cccc4c3Oc3ccccc3-c3cccc(-c5ccccc5)c3-4)cc21 ; c1ccc(-c2ccc(N(c3ccc(-c4ccccc4)cc3)c3cccc4c3Oc3ccccc3-c3cccc(-c5ccccc5)c3-4)cc2)cc1]

[chemical structure]

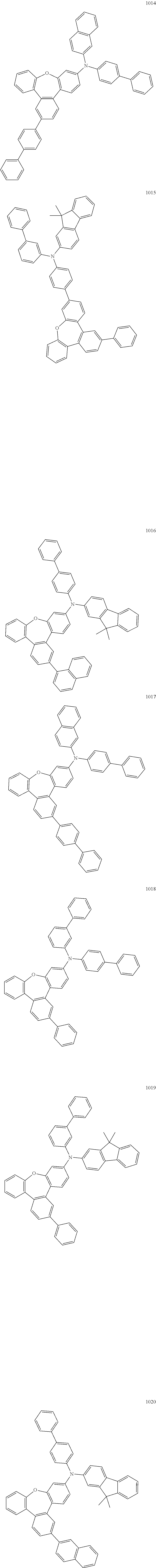

[chemical structure]

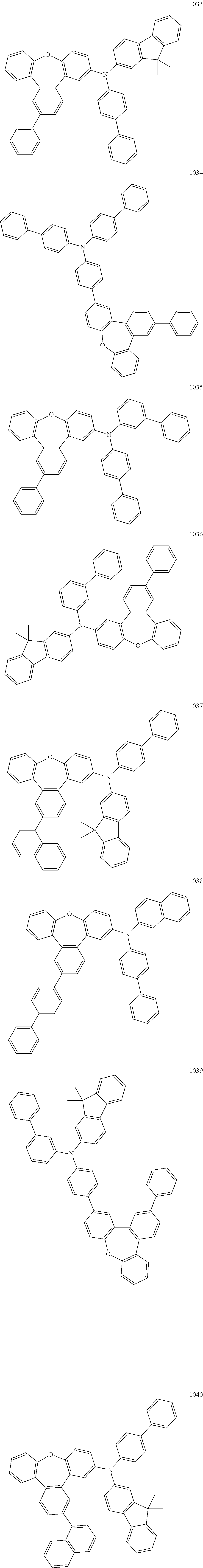

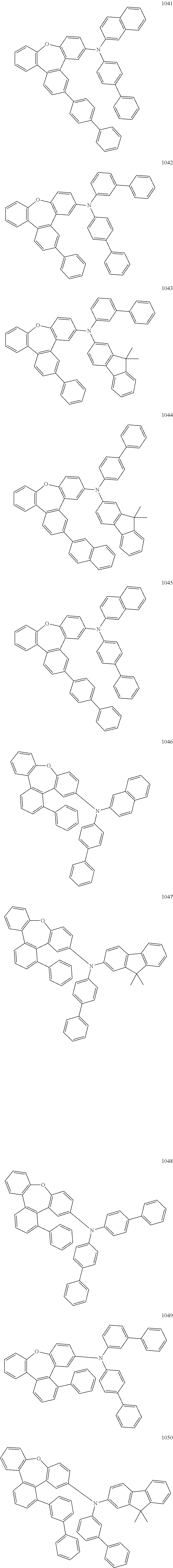

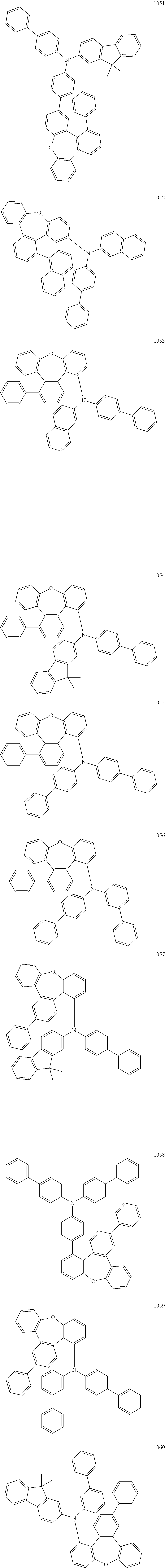

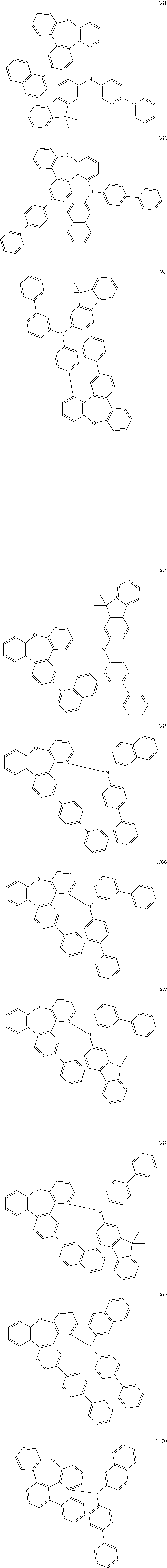

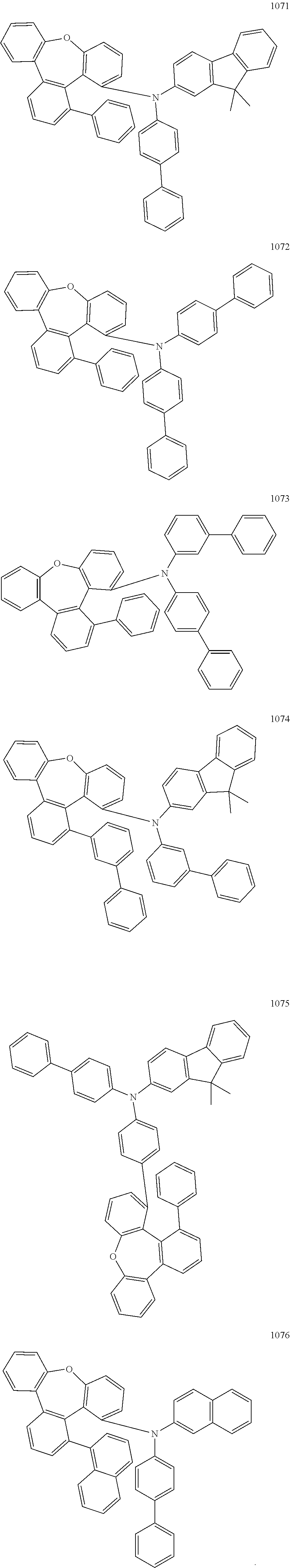

Further, compounds having intrinsic properties of the introduced substituents may be synthesized by introducing various substituents to the structure of Chemical Formula 1 above. For example, materials satisfying conditions required in each organic material layer may be synthesized by introducing substituents mainly used for a hole injection layer material, a hole transport layer material, an electron blocking layer material, a light emitting layer material, an electron transport layer material, a hole blocking layer material, and an electron injection layer material used during manufacturing of an organic light emitting device to the core structure.

Further, various substituents are introduced to the structure of Chemical Formula 1 above so that the energy band gap may be enabled to be finely controlled, whereas the properties in the interface between organic materials may be improved, and the use of the materials may be diversified.

Further, various substituents are introduced to the structure of Chemical Formula 1 above so that the characteristics of the hole are strengthened, and the band gap and triplet energy level (T₁ level) values may be enabled to be controlled, thereby increasing the energy level control possibility and diversifying the use of the materials.

Meanwhile, the compound represented by Chemical Formula 1 above has excellent thermal stability due to a high glass transition temperature (T_{g}). An increase in such thermal stability becomes an important factor in providing driving stability to the device.

Further, in one embodiment of the present disclosure, there is provided an organic light emitting device comprising: a first electrode; a second electrode provided opposite to the first electrode; and one or more organic material layers provided between the first electrode and the second electrode, wherein one or more layers of the organic material layers contain the heterocyclic compound represented by Chemical Formula 1 above.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic material layers may include one or more selected from the group consisting of an electron injection layer, an electron transport layer, a hole blocking layer, a light emitting layer, an electron blocking layer, a hole transport layer, and a hole injection layer, and one or more layers selected from the group consisting of an electron injection layer, an electron transport layer, a hole blocking layer, a light emitting layer, an electron blocking layer, a hole transport layer, and a hole injection layer may contain the heterocyclic compound represented by Chemical Formula 1 above.

In another embodiment of the present disclosure, the organic material layers may include a hole transport layer, and the hole transport layer may contain the heterocyclic compound represented by Chemical Formula 1 above.

In another embodiment of the present disclosure, the organic material layers may include an electron blocking layer, and the electron blocking layer may contain the heterocyclic compound represented by Chemical Formula 1 above.

In one embodiment of the present disclosure, the organic light emitting device may be a blue organic light emitting device, and the heterocyclic compound represented by Chemical Formula 1 above may be used as a material of the blue organic light emitting device.

In one embodiment of the present disclosure, the organic light emitting device may be a green organic light emitting device, and the heterocyclic compound represented by Chemical Formula 1 above may be used as a material of the green organic light emitting device.

In one embodiment of the present disclosure, the organic light emitting device may be a red organic light emitting device, and the heterocyclic compound represented by Chemical Formula 1 above may be used as a material of the red organic light emitting device.

In one embodiment of the present disclosure, the organic light emitting device may be a blue organic light emitting device, and the heterocyclic compound represented by Chemical Formula 1 above may be used as a material of a hole transport layer or an electron blocking layer of the blue organic light emitting device.

In one embodiment of the present disclosure, the organic light emitting device may be a green organic light emitting device, and the heterocyclic compound represented by Chemical Formula 1 above may be used as a material of a hole transport layer or an electron blocking layer of the green organic light emitting device.

In one embodiment of the present disclosure, the organic light emitting device may be a red organic light emitting device, and the heterocyclic compound represented by Chemical Formula 1 above may be used as a material of a hole transport layer or an electron blocking layer of the red organic light emitting device.

Specific details for the heterocyclic compound represented by Chemical Formula 1 above are the same as described above.

In an organic light emitting device according to one embodiment of the present disclosure, the organic material layers may include an electron injection layer or an electron transport layer, and the electron injection layer or the electron transport layer may contain the heterocyclic compound.

In an organic light emitting device according to another embodiment, the organic material layers may include an electron blocking layer or a hole blocking layer, and the electron blocking layer or the hole blocking layer may contain the heterocyclic compound.

In an organic light emitting device according to another embodiment, the organic material layers may include an electron transport layer, a light emitting layer, or a hole blocking layer, and the electron transport layer, the light emitting layer, or the hole blocking layer may contain the heterocyclic compound.

In an organic light emitting device according to another embodiment, the organic material layers may include a hole transport layer or an electron blocking layer, and the hole transport layer or the electron blocking layer may contain the heterocyclic compound.

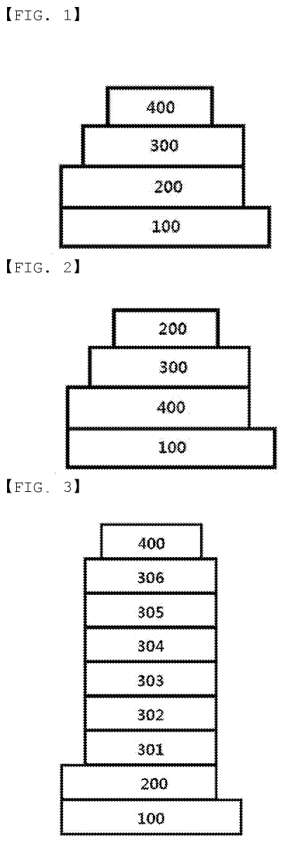

FIGS. 1 to 3 illustrate lamination orders of electrodes and organic material layers of an organic light emitting device according to one embodiment of the present disclosure. However, it is not intended that the scope of the present application be limited by these drawings, and the structure of an organic light emitting device known in the art may also be applied to the present application.

Referring to FIG. 1, an organic light emitting device in which a positive electrode 200, an organic material layer 300, and a negative electrode 400 are sequentially laminated on a substrate 100 is illustrated. However, it is not limited to such a structure only, and as shown in FIG. 2, an organic light emitting device in which a negative electrode, an organic material layer, and a positive electrode are sequentially laminated on a substrate may also be implemented.

FIG. 3 illustrates a case in which the organic material layer is multilayers. The organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by such a lamination structure, and if necessary, the remaining layers except for the light emitting layer may be omitted, and other necessary functional layers may be further added.

Further, one embodiment of the present disclosure provides a composition for an organic material layer of an organic light emitting device, comprising the heterocyclic compound represented by Chemical Formula 1 above.

Specific details for the heterocyclic compound represented by Chemical Formula 1 above are the same as described above.

The composition for an organic material layer of an organic light emitting device may be used when forming an organic material of the organic light emitting device, and particularly, it may be more preferably used when forming a hole transport layer or an electron blocking layer.

An organic light emitting device according to the present disclosure may be manufactured by a conventional method and material for manufacturing an organic light emitting device except that one or more organic material layers are formed using the above-described heterocyclic compound.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating, or the like, but is not limited thereto.

The organic material layer of the organic light emitting device according to the present disclosure may be formed in a single layer structure, but may also be formed in a multilayer structure in which two or more organic material layers are laminated. For example, the organic light emitting device according to the present disclosure may have a structure including an electron injection layer, an electron transport layer, a hole blocking layer, a light emitting layer, an electron blocking layer, an electron transport layer, an electron injection layer, and the like as the organic material layer. However, the structure of the organic light emitting device is not limited thereto, and may include a smaller number of organic material layers.

In one embodiment of the present disclosure, there is provided a method for manufacturing an organic light emitting device, the method comprising steps of: preparing a substrate; forming a first electrode on the substrate; forming one or more organic material layers on the first electrode; and forming a second electrode on the organic material layers, wherein the step of forming the organic material layers includes a step of forming one or more organic material layers using a composition for an organic material layer according to one embodiment of the present disclosure.

In an organic light emitting device according to one embodiment of the present disclosure, materials other than the heterocyclic compound represented by Chemical Formula 1 above are exemplified below, but these are for illustration only and not for limiting the scope of the present application, and may be replaced by materials known in the art.

As the positive electrode material, materials having a relatively large work function may be used, and transparent conductive oxides, metals, conductive polymers, or the like may be used. Specific examples of the positive electrode material include metals such as vanadium, chromium, copper, zinc, and gold, or alloys thereof; metal oxides such as zinc oxide, indium oxide, indium tin oxide (ITO), and indium zinc oxide (IZO); ZnO: Al or SnO₂: combinations of metals such as Sb, and oxides; conductive polymers such as poly(3-methylthiophene), poly[3,4-(ethylene-1,2-dioxy)thiophene] (PEDOT), polypyrrole, and polyaniline; and the like, but are not limited thereto.

As the negative electrode material, materials having a relatively low work function may be used, and metals, metal oxides, conductive polymers, or the like may be used. Specific examples of the negative electrode material include metals such as magnesium, calcium, sodium, potassium, titanium, indium, yttrium, lithium, gadolinium, aluminum, silver, tin, and lead, or alloys thereof; a multilayered material such as LiF/Al or LiO₂/Al; and the like, but are not limited thereto.

As the hole injection layer material, known hole injection layer materials may also be used, and for example, phthalocyanine compounds such as copper phthalocyanine and the like disclosed in U.S. Pat. No. 4,356,429, or starburst-type amine derivatives described in the literature [Advanced Material, 6, p. 677 (1994)] such as tris(4-carbazoyl-9-ylphenyl)amine (TCTA), 4,4′,4″-tris[phenyl(m-tolyl)amino]triphenylamine (m-MTDATA), 1,3,5-tris[4-(3-methylphenylphenylamino)phenyl]benzene (m-MTDAPB), a soluble conductive polymer of polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like may be used.

As the hole transport layer material, pyrazoline derivatives, arylamine-based derivatives, stilbene derivatives, triphenyldiamine derivatives, and the like may be used, and low molecular weight or high molecular weight materials may also be used.

As the electron transport layer material, metal complexes or the like of oxadiazole derivatives, anthraquinodimethane and its derivatives, and benzoquinone its derivatives, naphthoquinone and its derivatives, anthraquinone and its derivatives, tetracyanoanthraquinodimethane and its derivatives, fluorenone derivatives, diphenyldicyanoethylene and its derivatives, diphenoquinone derivatives, and 8-hydroxyquinoline and its derivatives may be used, and high molecular weight materials as well as low molecular weight materials may also be used.

As the electron injection layer material, for example, LiF is typically used in the art, but the present application is not limited thereto.

As the light emitting layer material, a red, green, or blue light emitting material may be used, and two or more light emitting materials may be mixed and used if necessary. At this time, two or more light emitting materials may be deposited and used as individual sources, or may be premixed to be deposited and used as one source. In addition, as the light emitting layer material, a fluorescent material may be used, but a phosphorescent material may also be used. As the light emitting layer material, a material that emits light by combining holes and electrons respectively injected from the positive electrode and the negative electrode may be used alone, but materials in which the host material and the dopant material involve together in light emission may also be used.

When the host of the light emitting layer material is mixed and used, a host of the same series may be mixed and used, or a host of different series may also be mixed and used. For example, any two or more types of materials of n-type host materials or p-type host materials may be selected and used as a host material of the light emitting layer.

The organic light emitting device according to one embodiment of the present disclosure may be a top emission type, a back emission type, or a double side emission type depending on materials used.

The heterocyclic compound according to one embodiment of the present disclosure may act on a principle similar to that applied to an organic light emitting device even in organic electronic devices including an organic solar cell, an organic photoreceptor, an organic transistor, and the like.

### MODE FOR CARRYING OUT THE INVENTION

Hereinafter, preferred examples are presented to help the understanding of the present disclosure, but the following Examples are only provided for easier understanding of the present disclosure, and the present disclosure is not limited thereto.

### PREPARATION EXAMPLES

### Preparation Example 1. Preparation of Compound 1

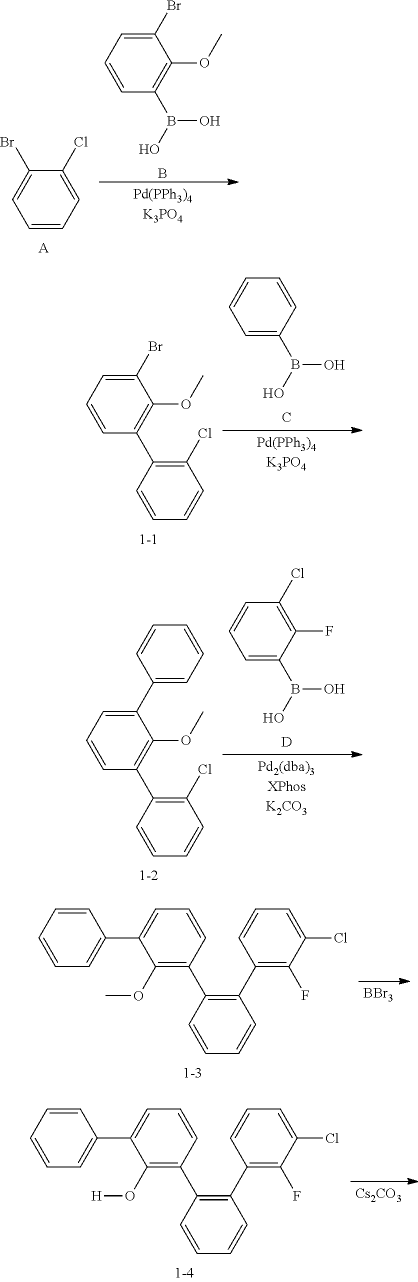

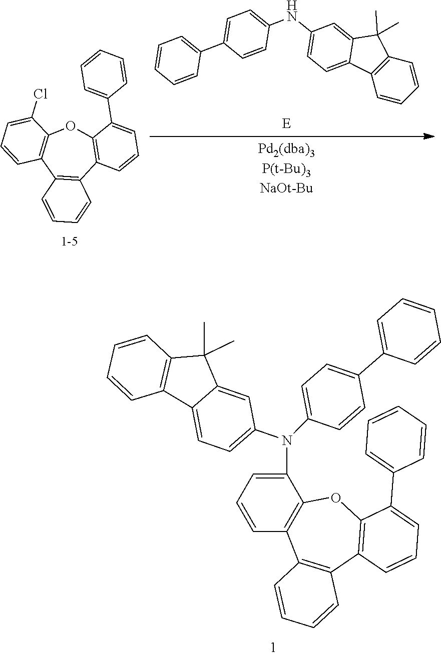

### 1) Preparation of Compound 1-1

1-bromo-2-chlorobenzene (A) (30 g, 0.157 mol, 1 eq), (3-bromo-2-methoxyphenyl)boronic acid (B) (39.9 g, 0.173 mol, 1.1 eq), tetrakis(triphenylphosphine)palladium (Pd(PPh₃)₄) (9.2 g, 0.008 mol, 0.05 eq), and potassium triphosphate (K₃PO₄) (73.2 g, 0.345 mol, 2.2 eq) were added to 1,4-dioxane (360 ml) and water (60 ml), and stirred at 100° C. for 12 hours. After completion of the reaction by adding water, extraction was performed using methylene chloride (MC) and water. Thereafter, water was removed from the extract with magnesium sulfate (MgSO₄), and then the water-removed extract was separated by a silica gel column to obtain 33 g of Compound 1-1 (yield 95%).

### 2) Preparation of Compound 1-2

Compound 1-1 (33 g, 0.111 mol, 1 eq), phenylboronic acid (C) (14.9 g, 0.122 mol, 1.1 eq), tetrakis(triphenylphosphine)palladium (Pd(PPh₃)₄) (6.9 g, 0.006 mol, 0.05 eq), and potassium triphosphate (K₃PO₄) (51.8 g, 0.244 mol, 2.2 eq) were added to 1,4-dioxane (396 ml) and water (99 ml), and stirred at 100° C. for 6 hours. After completion of the reaction by adding water, extraction was performed using methylene chloride (MC) and water. Thereafter, water was removed from the extract with magnesium sulfate (MgSO₄), and then the water-removed extract was separated by a silica gel column to obtain 30.1 g of Compound 1-2 (yield 93%).

### 3) Preparation of Compound 1-3

Compound 1-2 (30 g, 0.102 mol, 1 eq), (3-chloro-2-fluorophenyl)boronic acid (D) (26.7 g, 0.153 mol, 1.5 eq), tris(dibenzylideneacetone)dipalladium (Pd₂(dba)₃) (4.6 g, 0.005 mol, 0.05 eq), XPhos (4.8 g, 0.010 mol, 0.1 eq), and potassium carbonate (K₂CO₃) (42.2 g, 0.306 mol, 3.0 eq) were added to 1,4-dioxane (360 ml) and water (90 ml), and stirred at 100° C. for 24 hours. After completion of the reaction by adding water, extraction was performed using methylene chloride (MC) and water. Thereafter, water was removed from the extract with magnesium sulfate (MgSO₄), and then the water-removed extract was separated by a silica gel column to obtain 22.2 g of Compound 1-3 (yield 70%).

### 4) Preparation of Compound 1-4

Compound 1-3 (22 g, 0.057 mol, 1 eq) was added to methylene chloride (MC) (220 ml) and cooled while stirring it at 0° C. Thereafter, boron tribromide (BBr₃) (28.6 g, 0.114 mol, 2.0 eq) was slowly added dropwise, and then stirred at room temperature for 1 hour. After completion of the reaction by adding water, extraction was performed using methylene chloride (MC) and water. Thereafter, water was removed from the extract with magnesium sulfate (MgSO₄), and then the water-removed extract was separated by a silica gel column to obtain 19.5 g of Compound 1-4 (yield 95%).

### 5) Preparation of Compound 1-5

Compound 1-4 (19 g, 0.051 mol, 1 eq) and cesium carbonate (Cs₂CO₃) (33.2 g, 0.102 mol, 2.0 eq) were added to dimethylacetamide (DMA) (285 ml), and stirring was performed for 1 hour while refluxing the mixture. After completion of the reaction by adding water, extraction was performed using methylene chloride (MC) and water. Thereafter, water was removed from the extract with magnesium sulfate (MgSO₄), and then the water-removed extract was separated by a silica gel column to obtain 16.3 g of Compound 1-5 (yield 95%).

### 6) Preparation of Compound 1

Compound 1-5 (8 g, 0.023 mol, 1 eq), N-([1,1′-biphenyl]-4-yl)-9,9-dimethyl-9H-fluoren-2-amine (E) (9.0 g, 0.025 mol, 1.1 eq), tris(dibenzylideneacetone)dipalladium (Pd₂(dba)₃) (0.9 g, 0.001 mol, 0.05 eq), tri-tert-butylphosphine (P(t-Bu)₃) (50% in Toluene) (0.8 g, 0.002 mol, 0.1 eq), and sodium tert-butoxide (NaOt-Bu) (4.4 g, 0.046 mol, 2.0 eq) were added to toluene (80 ml), and stirred at 100° C. for 2 hours. After completion of the reaction by adding water, extraction was performed using methylene chloride (MC) and water. Thereafter, water was removed from the extract with magnesium sulfate (MgSO₄), and then the water-removed extract was separated by a silica gel column to obtain 12.2 g of Compound 1 (yield 80%).

The compounds were synthesized in the same manner as in Preparation Example 1 above by using Intermediate A of Table 1 below instead of 1-bromo-2-chlorobenzene (A), using Intermediate B of Table 1 below instead of (3-bromo-2-methoxyphenyl)boronic acid (B), using Intermediate C of Table 1 below instead of phenylboronic acid (C), using Intermediate D of Table 1 below instead of (3-chloro-2-fluorophenyl)boronic acid (D), and using Intermediate E of Table 1 below instead of N-([1,1′-biphenyl]-4-yl)-9,9-dimethyl-9H-fluoren-2-amine (E) in Preparation Example 1 above.

TABLE 1

| Compound number | Intermediate A | Intermediate B | Intermediate C | Intermediate D | Intermediate E | Yield |
|---|---|---|---|---|---|---|
| 1 | 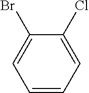 | 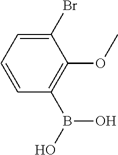 | 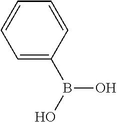 | 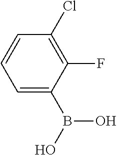 [SMILES: OB(O)c1cccc(Cl)c1F] | 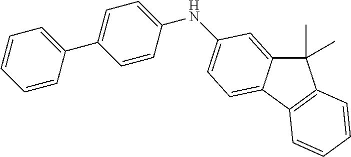 [SMILES: CC1(C)c2ccccc2-c2ccc(Nc3ccc(-c4ccccc4)cc3)cc21] | 45% |
| 120 | 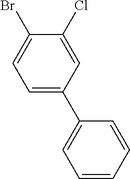 [SMILES: Clc1cc(-c2ccccc2)ccc1Br] | 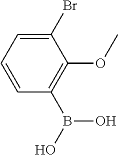 | 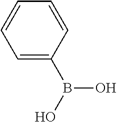 | 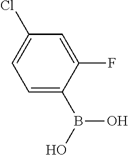 [SMILES: OB(O)c1ccc(Cl)cc1F] | 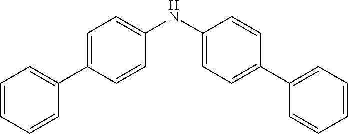 [SMILES: c1ccc(-c2ccc(Nc3ccc(-c4ccccc4)cc3)cc2)cc1] | 50% |
| 197 | 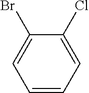 | 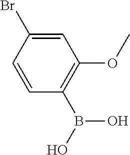 | 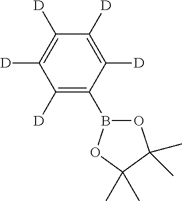 [SMILES: [2H]c1c([2H])c([2H])c(B2OC(C)(C)C(C)(C)O2)c([2H])c1[2H]] | 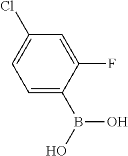 [SMILES: OB(O)c1ccc(Cl)cc1F] | 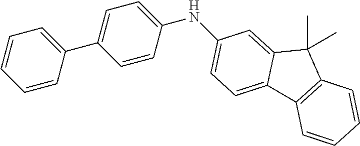 [SMILES: CC1(C)c2ccccc2-c2ccc(Nc3ccc(-c4ccccc4)cc3)cc21] | 48% |
| 228 | 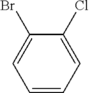 | 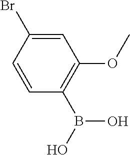 | 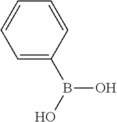 | 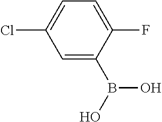 [SMILES: OB(O)c1cc(Cl)ccc1F] | 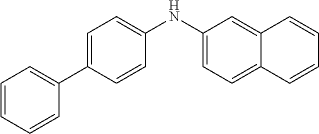 [SMILES: c1ccc(-c2ccc(Nc3ccc4ccccc4c3)cc2)cc1] | 49% |
| 233 | 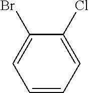 | 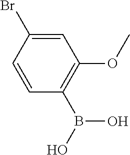 | 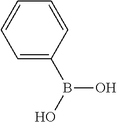 | 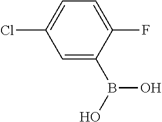 [SMILES: OB(O)c1cc(Cl)ccc1F] | 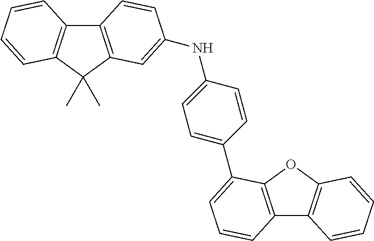 [SMILES: CC1(C)c2ccccc2-c2ccc(Nc3ccc(-c4cccc5c4oc4ccccc45)cc3)cc21] | 49% |
| 246 | 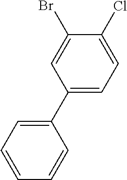 [SMILES: Clc1ccc(-c2ccccc2)cc1Br] | 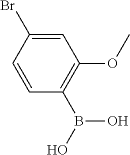 | 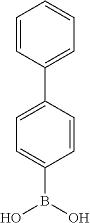 [SMILES: OB(O)c1ccc(-c2ccccc2)cc1] | 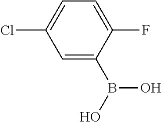 [SMILES: OB(O)c1cc(Cl)ccc1F] | 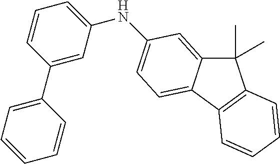 [SMILES: CC1(C)c2ccccc2-c2ccc(Nc3cccc(-c4ccccc4)c3)cc21] | 48% |
| 237 | 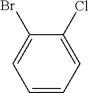 | 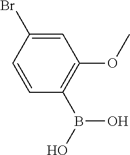 | 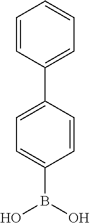 [SMILES: OB(O)c1ccc(-c2ccccc2)cc1] | 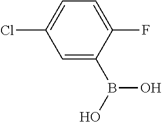 [SMILES: OB(O)c1cc(Cl)ccc1F] | 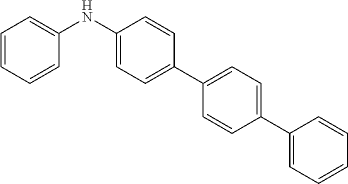 [SMILES: c1ccc(Nc2ccc(-c3ccc(-c4ccccc4)cc3)cc2)cc1] | 50% |
| 508 | 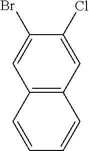 [SMILES: Clc1cc2ccccc2cc1Br] | 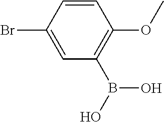 | 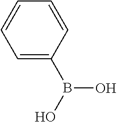 | 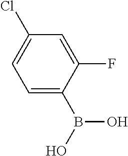 [SMILES: OB(O)c1ccc(Cl)cc1F] | 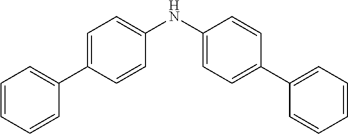 [SMILES: c1ccc(-c2ccc(Nc3ccc(-c4ccccc4)cc3)cc2)cc1] | 50% |
| 514 | 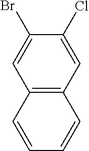 [SMILES: Clc1cc2ccccc2cc1Br] | 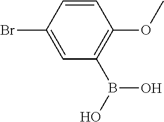 | 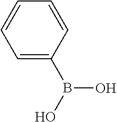 | 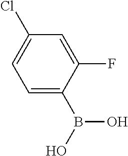 [SMILES: OB(O)c1ccc(Cl)cc1F] | 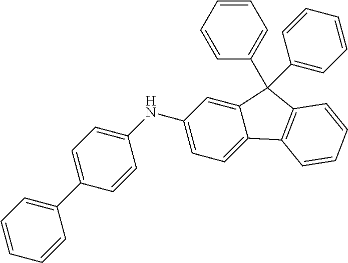 [SMILES: c1ccc(-c2ccc(Nc3ccc4c(c3)C(c3ccccc3)(c3ccccc3)c3ccccc3-4)cc2)cc1] | 50% |
| 562 | 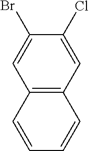 [SMILES: Clc1cc2ccccc2cc1Br] | 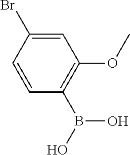 | 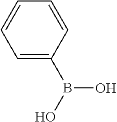 | 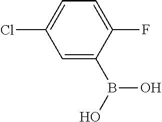 [SMILES: OB(O)c1cc(Cl)ccc1F] | 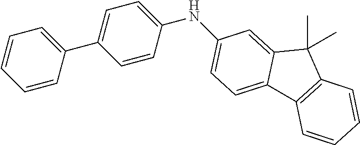 [SMILES: CC1(C)c2ccccc2-c2ccc(Nc3ccc(-c4ccccc4)cc3)cc21] | 48% |

### Preparation Example 2: Preparation of Compound 16

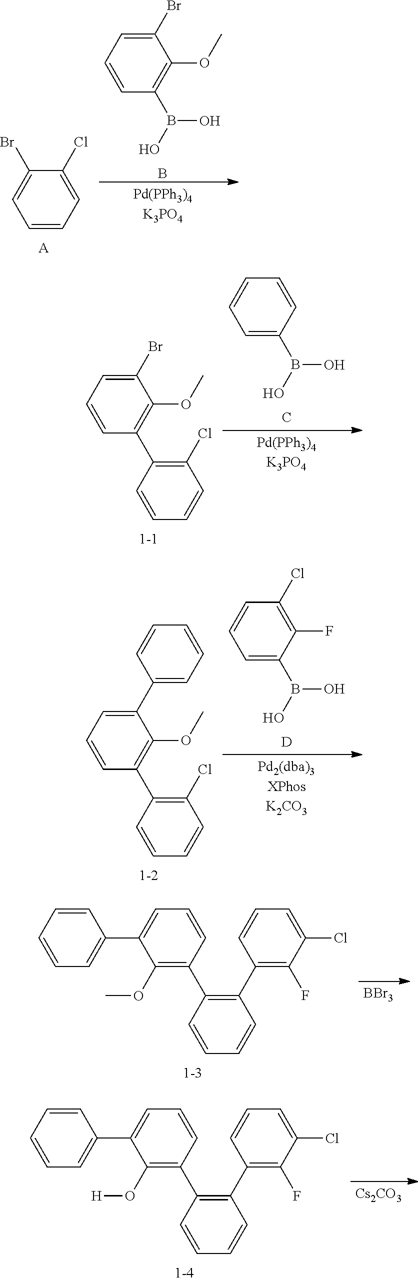

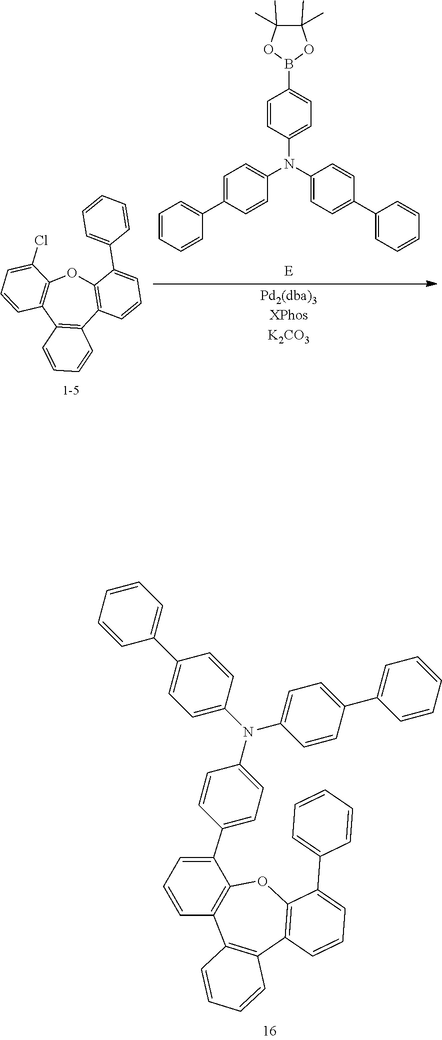

Compounds were synthesized in Preparation Example 2 in the same manner as in the preparation of Compound 1-1 to Compound 1-5 in Preparation Example 1 above.

### 1) Preparation of Compound 16

Compound 1-5 (8 g, 0.023 mol, 1 eq), N-([1,1′-biphenyl]-4-yl)-N-(4-(4,4,5,5-tetramethyl-1,3,2-dioxaborolan-2-yl)phenyl)-[1,1′-biphenyl]-4-amine (E) (13.1 g, 0.025 mol, 1.1 eq), tris(dibenzylideneacetone)dipalladium (Pd₂(dba)₃) (0.9 g, 0.001 mol, 0.05 eq), Xphos (1.0 g, 0.002 mol, 0.1 eq), and potassium carbonate (K₂CO₃) (9.5 g, 0.046 mol, 3.0 eq) were added to toluene (80 ml), and stirred at 100° C. for 6 hours. After completion of the reaction by adding water, extraction was performed using methylene chloride (MC) and water. Thereafter, water was removed from the extract with magnesium sulfate (MgSO₄), and then the water-removed extract was separated by a silica gel column to obtain 10.7 g of Compound 16 (yield 75%).

The compounds were synthesized in the same manner as in Preparation Example 2 above by using Intermediate A of Table 2 below instead of 1-bromo-2-chlorobenzene (A), using Intermediate B of Table 2 below instead of (3-bromo-2-methoxyphenyl)boronic acid (B), using Intermediate C of Table 2 below instead of phenylboronic acid (C), using Intermediate D of Table 2 below instead of (3-chloro-2-fluorophenyl)boronic acid (D), and using Intermediate E of Table 2 below instead of N-([1,1′-biphenyl]-4-yl)-N-(4-(4,4,5,5-tetramethyl-1,3,2-dioxaborolan-2-yl)phenyl)-[1,1′-biphenyl]-4-amine (E) in Preparation Example 2 above.

TABLE 2

| Compound number | Intermediate A | Intermediate B | Intermediate C | Intermediate D | Intermediate E | Yield |
|---|---|---|---|---|---|---|
| 16 | 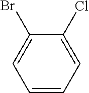 | 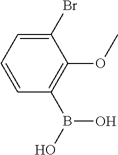 | 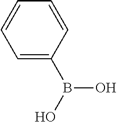 | 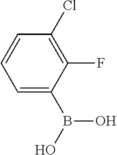 [SMILES: OB(O)c1cccc(Cl)c1F] | 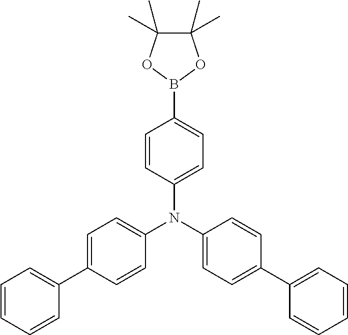 [SMILES: CC1(C)OB(c2ccc(N(c3ccc(-c4ccccc4)cc3)c3ccc(-c4ccccc4)cc3)cc2)OC1(C)C] | 42% |
| 23 | 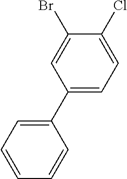 [SMILES: Clc1ccc(-c2ccccc2)cc1Br] | 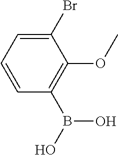 | 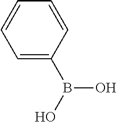 | 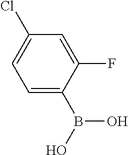 [SMILES: OB(O)c1ccc(Cl)cc1F] | 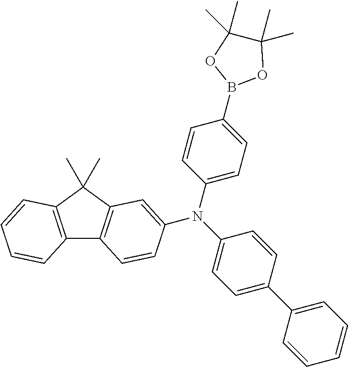 [SMILES: CC1(C)c2ccccc2-c2ccc(N(c3ccc(B4OC(C)(C)C(C)(C)O4)cc3)c3ccc(-c4ccccc4)cc3)cc21] | 41% |
| 217 | 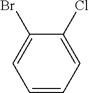 | 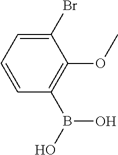 | 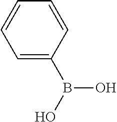 | 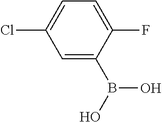 [SMILES: OB(O)c1cc(Cl)ccc1F] | 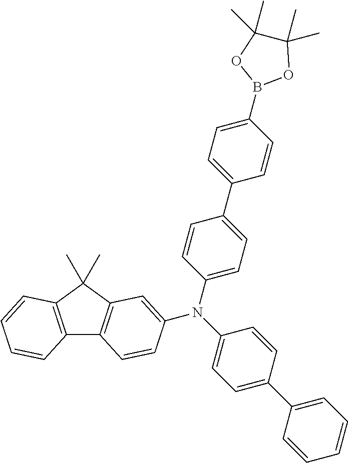 [SMILES: CC1(C)c2ccccc2-c2ccc(N(c3ccc(-c4ccccc4)cc3)c3ccc(-c4ccc(B5OC(C)(C)C(C)(C)O5)cc4)cc3)cc21] | 42% |
| 522 | 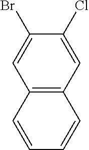 [SMILES: Clc1cc2ccccc2cc1Br] | 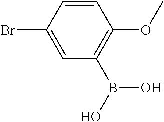 | 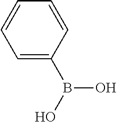 | 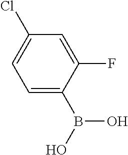 [SMILES: OB(O)c1ccc(Cl)cc1F] | 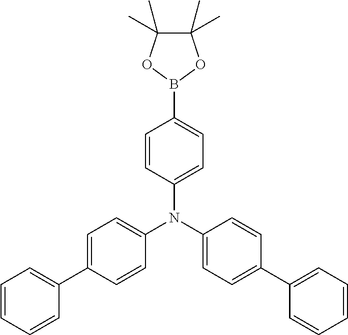 [SMILES: CC1(C)OB(c2ccc(N(c3ccc(-c4ccccc4)cc3)c3ccc(-c4ccccc4)cc3)cc2)OC1(C)C] | 42% |
| 541 | 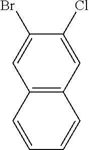 [SMILES: Clc1cc2ccccc2cc1Br] | 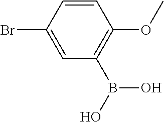 | 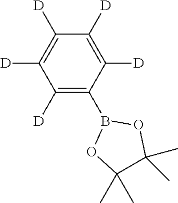 [SMILES: [2H]c1c([2H])c([2H])c(B2OC(C)(C)C(C)(C)O2)c([2H])c1[2H]] | 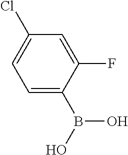 [SMILES: OB(O)c1ccc(Cl)cc1F] | 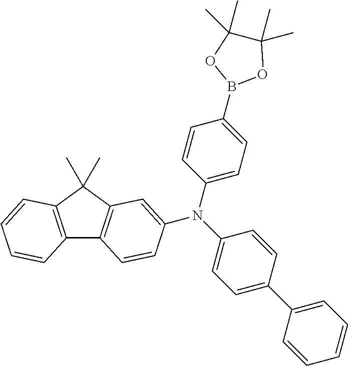 [SMILES: CC1(C)c2ccccc2-c2ccc(N(c3ccc(B4OC(C)(C)C(C)(C)O4)cc3)c3ccc(-c4ccccc4)cc3)cc21] | 41% |
| 578 | 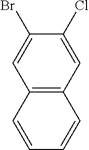 [SMILES: Clc1cc2ccccc2cc1Br] | 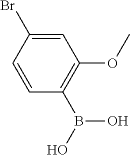 | 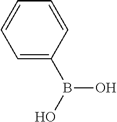 | 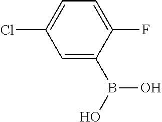 [SMILES: OB(O)c1cc(Cl)ccc1F] | 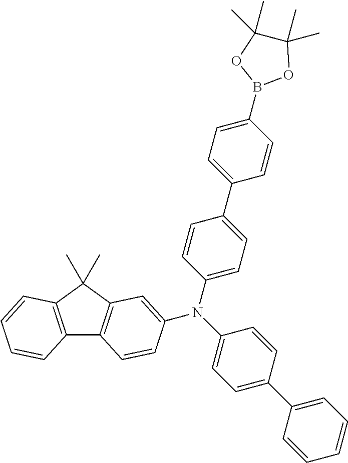 [SMILES: CC1(C)c2ccccc2-c2ccc(N(c3ccc(-c4ccccc4)cc3)c3ccc(-c4ccc(B5OC(C)(C)C(C)(C)O5)cc4)cc3)cc21] | 41% |

### Preparation Example 3: Preparation of Compound 985

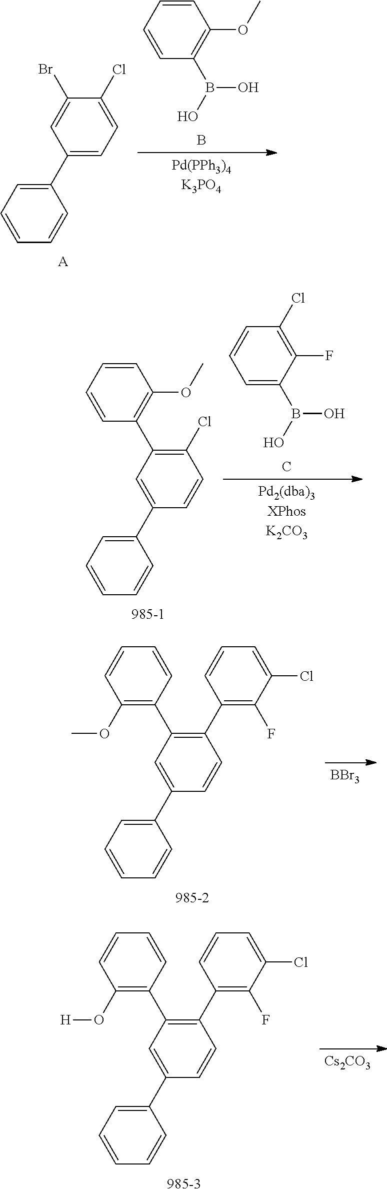

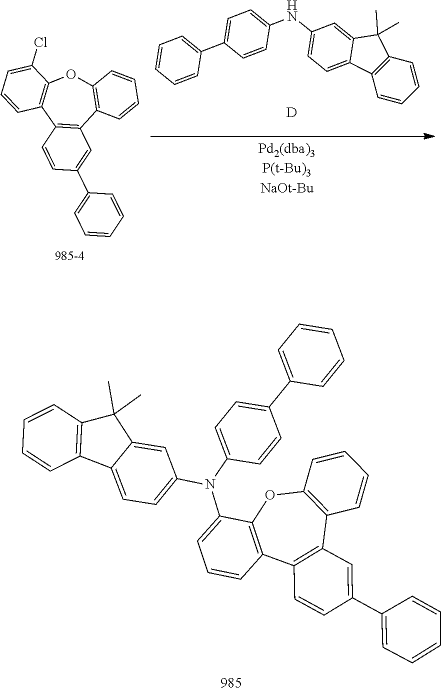

Compound 985 was synthesized in Preparation Example 3 in the same manner as in the preparation of Compound 1-1, Compound 1-3 to Compound 1-5, and Compound 1 in Preparation Example 1 above.

The compounds were synthesized in the same manner as in Preparation Example 3 above by using Intermediate A of Table 3 below instead of 3-bromo-4-chloro-1,1′-biphenyl (A), using Intermediate B of Table 3 below instead of (2-methoxyphenyl)boronic acid (B), using Intermediate C of Table 3 below instead of (3-chloro-2-fluorophenyl)boronic acid (C), and using Intermediate D of Table 3 below instead of N-([1,1′-biphenyl]-4-yl)-9,9-dimethyl-9H-fluoren-2-amine (D) in Preparation Example 3 above.

TABLE 3

| Compound number | Intermediate A | Intermediate B | Intermediate C | Intermediate D | yield |
|---|---|---|---|---|---|
| 985 | 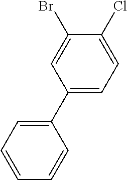 [SMILES: Clc1ccc(-c2ccccc2)cc1Br] | 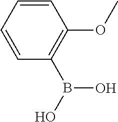 | 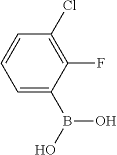 [SMILES: OB(O)c1cccc(Cl)c1F] | 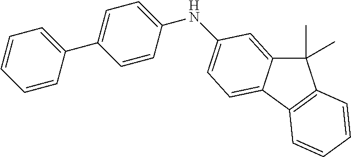 [SMILES: CC1(C)c2ccccc2-c2ccc(Nc3ccc(-c4ccccc4)cc3)cc21] | 50% |
| 199 | 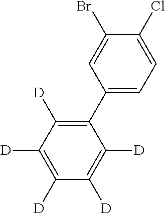 [SMILES: [2H]c1c([2H])c([2H])c(-c2ccc(Cl)c(Br)c2)c([2H])c1[2H]] | 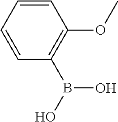 | 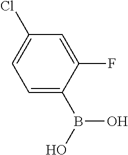 [SMILES: OB(O)c1ccc(Cl)cc1F] | 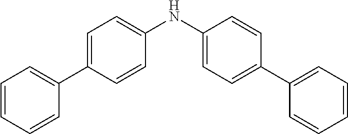 [SMILES: c1ccc(-c2ccc(Nc3ccc(-c4ccccc4)cc3)cc2)cc1] | 51% |
| 702 | 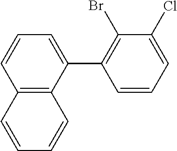 [SMILES: Clc1cccc(-c2cccc3ccccc23)c1Br] | 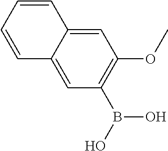 | 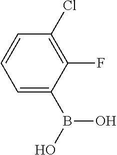 [SMILES: OB(O)c1cccc(Cl)c1F] | 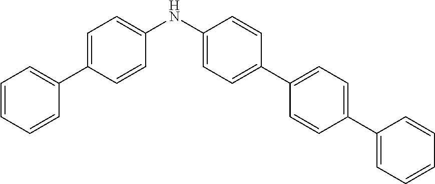 [SMILES: c1ccc(-c2ccc(Nc3ccc(-c4ccc(-c5ccccc5)cc4)cc3)cc2)cc1] | 51% |
| 708 | 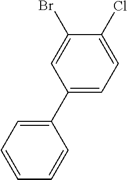 [SMILES: Clc1ccc(-c2ccccc2)cc1Br] | 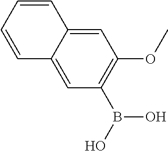 | 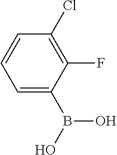 [SMILES: OB(O)c1cccc(Cl)c1F] | 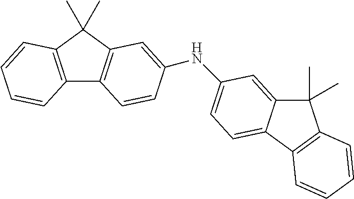 [SMILES: CC1(C)c2ccccc2-c2ccc(Nc3ccc4c(c3)C(C)(C)c3ccccc3-4)cc21] | 48% |
| 854 | 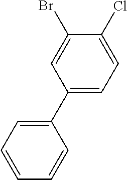 [SMILES: Clc1ccc(-c2ccccc2)cc1Br] | 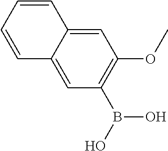 | 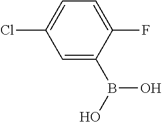 [SMILES: OB(O)c1cc(Cl)ccc1F] | 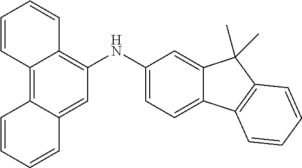 [SMILES: CC1(C)c2ccccc2-c2ccc(Nc3cc4ccccc4c4ccccc34)cc21] | 50% |
| 989 | 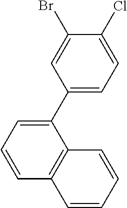 [SMILES: Clc1ccc(-c2cccc3ccccc23)cc1Br] | 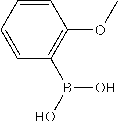 | 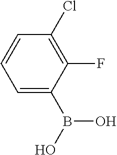 [SMILES: OB(O)c1cccc(Cl)c1F] | 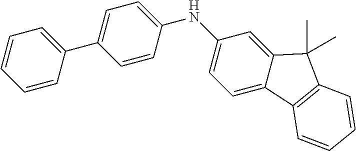 [SMILES: CC1(C)c2ccccc2-c2ccc(Nc3ccc(-c4ccccc4)cc3)cc21] | 49% |
| 1007 | 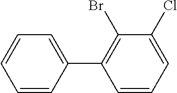 [SMILES: Clc1cccc(-c2ccccc2)c1Br] | 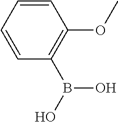 | 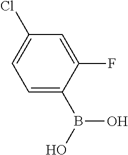 [SMILES: OB(O)c1ccc(Cl)cc1F] | 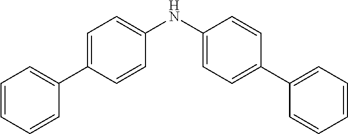 [SMILES: c1ccc(-c2ccc(Nc3ccc(-c4ccccc4)cc3)cc2)cc1] | 43% |
| 1011 | 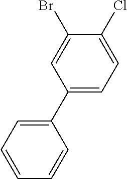 [SMILES: Clc1ccc(-c2ccccc2)cc1Br] | 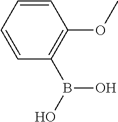 | 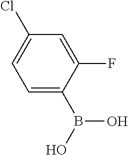 [SMILES: OB(O)c1ccc(Cl)cc1F] | 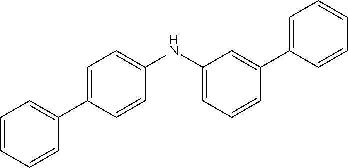 [SMILES: c1ccc(-c2ccc(Nc3cccc(-c4ccccc4)c3)cc2)cc1] | 49% |
| 1012 | 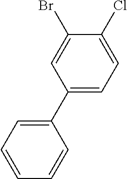 [SMILES: Clc1ccc(-c2ccccc2)cc1Br] | 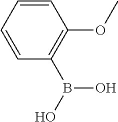 | 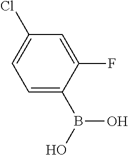 [SMILES: OB(O)c1ccc(Cl)cc1F] | 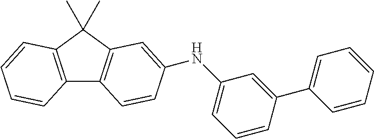 [SMILES: CC1(C)c2ccccc2-c2ccc(Nc3cccc(-c4ccccc4)c3)cc21] | 50% |
| 1046 | 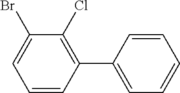 [SMILES: Clc1c(Br)cccc1-c1ccccc1] | 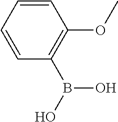 | 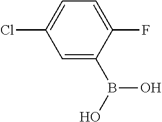 [SMILES: OB(O)c1cc(Cl)ccc1F] | 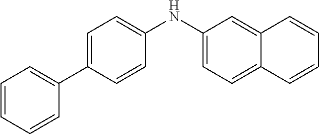 [SMILES: c1ccc(-c2ccc(Nc3ccc4ccccc4c3)cc2)cc1] | 40% |

### Preparation Example 4: Preparation of Compound 986

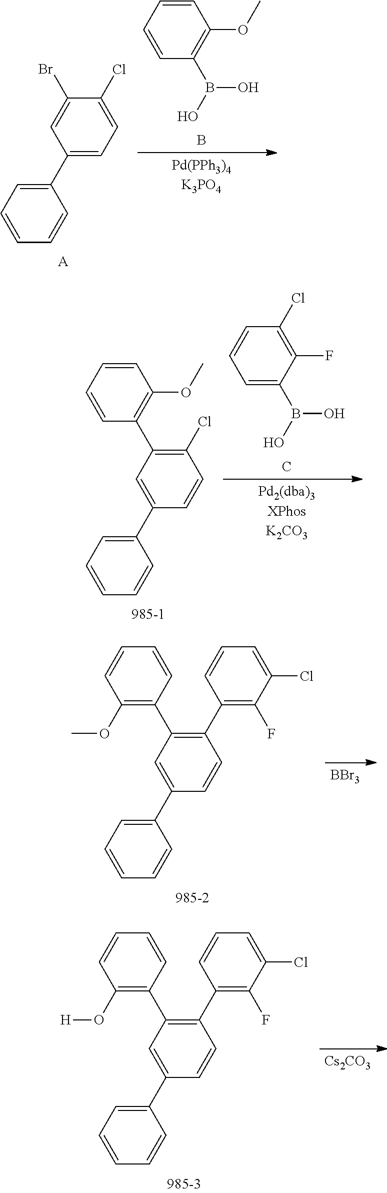

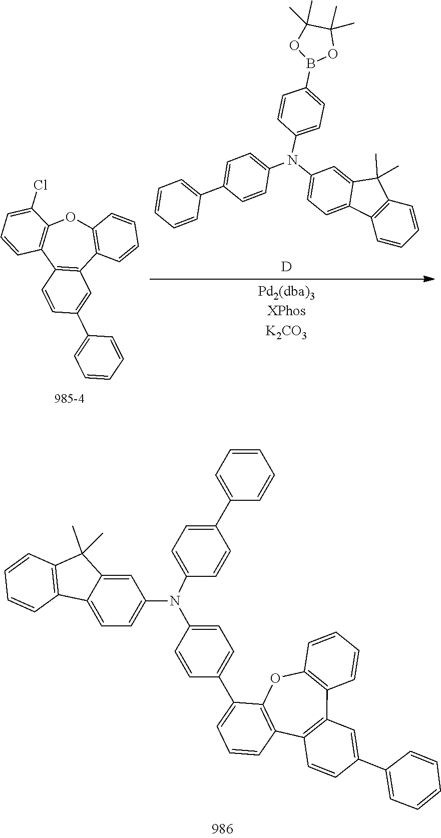

The preparation in Preparation Example 4 was performed in the same manner as in the preparation of Compounds 985-1 to 985-4 in Preparation Example 3 above, and Compound 986 was synthesized in the same manner as in the preparation of Compound 16 in Preparation Example 2 above.

The compounds were synthesized in the same manner as in Preparation Example 4 above by using Intermediate A of Table 4 below instead of 3-bromo-4-chloro-1,1′-biphenyl (A), using Intermediate B of Table 4 below instead of (2-methoxyphenyl)boronic acid (B), using Intermediate C of Table 4 below instead of (3-chloro-2-fluorophenyl)boronic acid (C), and using Intermediate D of Table 4 below instead of N-([1,1′-biphenyl]-4-yl)-9,9-dimethyl-N-(4-(4,4,5,5-tetramethyl-1,3,2-dioxaborolan-2-yl)phenyl)-9H-fluoren-2-amine (D) in Preparation Example 4 above.

TABLE 4

| Compound number | Intermediate A | Intermediate B | Intermediate C | Intermediate D | yield |
|---|---|---|---|---|---|
| 986 | 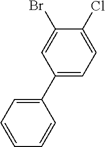 [SMILES: Clc1ccc(-c2ccccc2)cc1Br] | 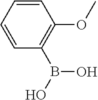 | 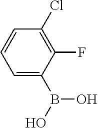 [SMILES: OB(O)c1cccc(Cl)c1F] | 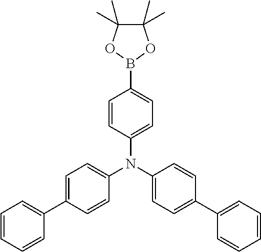 [SMILES: CC1(C)OB(c2ccc(N(c3ccc(-c4ccccc4)cc3)c3ccc(-c4ccccc4)cc3)cc2)OC1(C)C] | 48% |
| 721 | 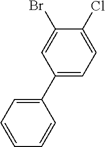 [SMILES: Clc1ccc(-c2ccccc2)cc1Br] | 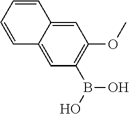 | 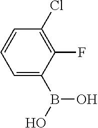 [SMILES: OB(O)c1cccc(Cl)c1F] | 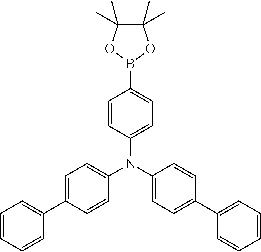 [SMILES: CC1(C)OB(c2ccc(N(c3ccc(-c4ccccc4)cc3)c3ccc(-c4ccccc4)cc3)cc2)OC1(C)C] | 47% |
| 828 | 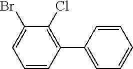 [SMILES: Clc1c(Br)cccc1-c1ccccc1] | 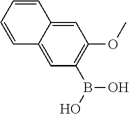 | 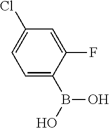 [SMILES: OB(O)c1ccc(Cl)cc1F] | 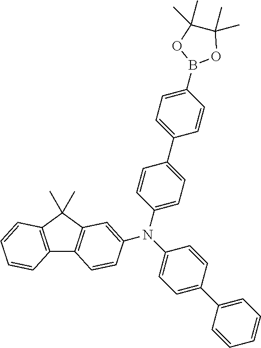 [SMILES: CC1(C)c2ccccc2-c2ccc(N(c3ccc(-c4ccccc4)cc3)c3ccc(-c4ccc(B5OC(C)(C)C(C)(C)O5)cc4)cc3)cc21] | 48% |
| 865 | 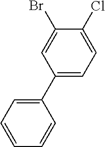 [SMILES: Clc1ccc(-c2ccccc2)cc1Br] | 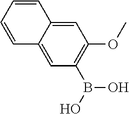 | 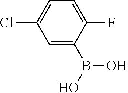 [SMILES: OB(O)c1cc(Cl)ccc1F] | 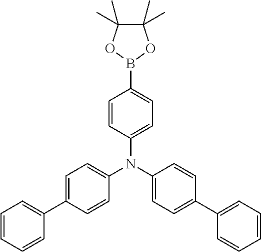 [SMILES: CC1(C)OB(c2ccc(N(c3ccc(-c4ccccc4)cc3)c3ccc(-c4ccccc4)cc3)cc2)OC1(C)C] | 50% |
| 1010 | 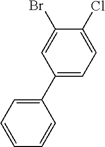 [SMILES: Clc1ccc(-c2ccccc2)cc1Br] | 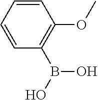 | 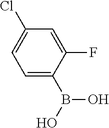 [SMILES: OB(O)c1ccc(Cl)cc1F] | 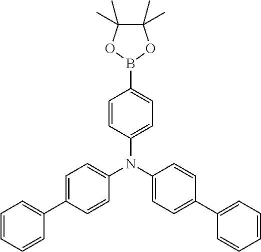 [SMILES: CC1(C)OB(c2ccc(N(c3ccc(-c4ccccc4)cc3)c3ccc(-c4ccccc4)cc3)cc2)OC1(C)C] | 50% |
| 1015 | 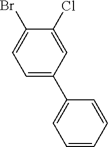 [SMILES: Clc1cc(-c2ccccc2)ccc1Br] | 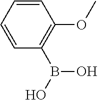 | 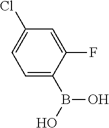 [SMILES: OB(O)c1ccc(Cl)cc1F] | 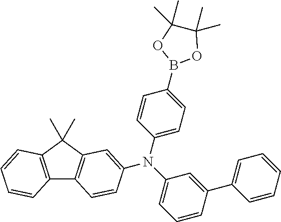 [SMILES: CC1(C)c2ccccc2-c2ccc(N(c3ccc(B4OC(C)(C)C(C)(C)O4)cc3)c3cccc(-c4ccccc4)c3)cc21] | 46% |
| 1051 | 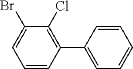 [SMILES: Clc1c(Br)cccc1-c1ccccc1] | 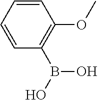 | 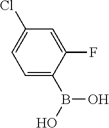 [SMILES: OB(O)c1ccc(Cl)cc1F] | 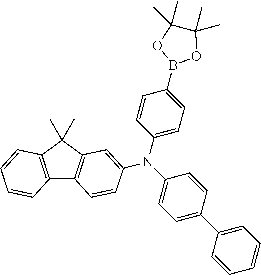 [SMILES: CC1(C)c2ccccc2-c2ccc(N(c3ccc(B4OC(C)(C)C(C)(C)O4)cc3)c3ccc(-c4ccccc4)cc3)cc21] | 45% |

### Preparation Example 5: Preparation of Compound 300

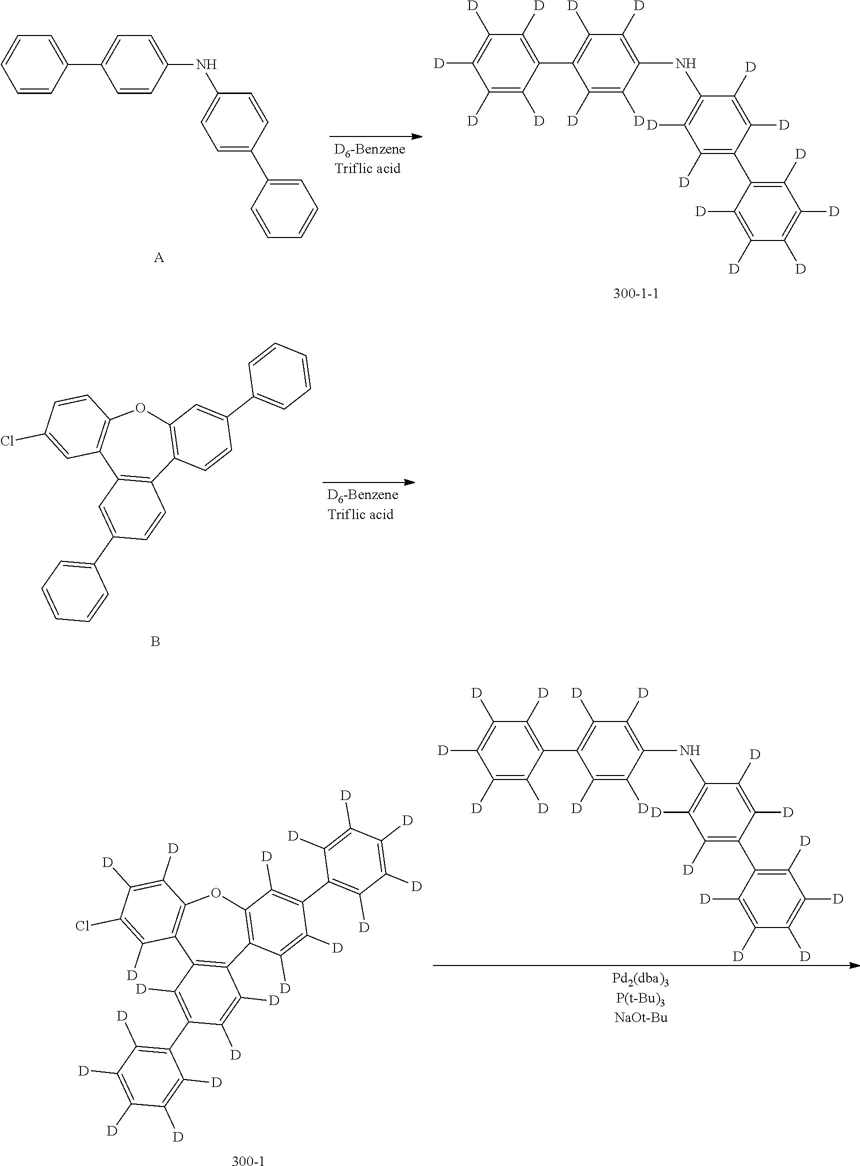

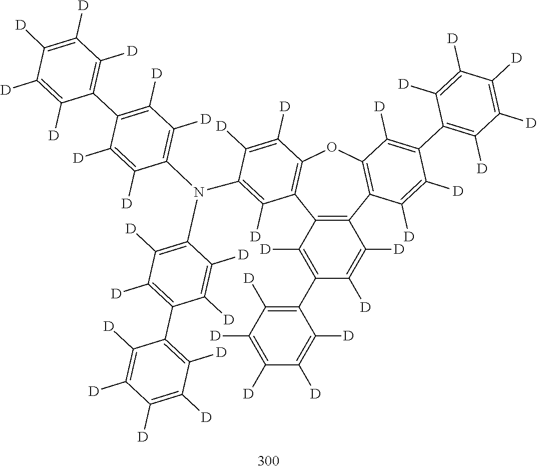 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])-c3c([2H])c(-c5c([2H])c([2H])c([2H])c([2H])c5[2H])c([2H])c([2H])c3-c3c([2H])c([2H])c(-c5c([2H])c([2H])c([2H])c([2H])c5[2H])c([2H])c3O4)c([2H])c2[2H])c([2H])c1[2H]]

Compound B in Preparation Example 5 is a compound synthesized by Preparation Example above, Compound 300-1 was synthesized in the same manner as in the preparation of Compound 300-1-1 below, and Compound 300 was synthesized in the same manner as in the preparation of Compound 1 in Preparation Example 1 above.

### 1) Preparation of Compound 300-1-1

Di([1,1′-biphenyl]-4-yl)amine (A) (30 g, 0.093 mol, 1 eq) was added to D₆-benzene (300 ml) and triflic acid (57.5 ml, 0.651 mol, 7.0 eq), and stirred at 60° C. for 1 hour. After completion of the reaction by adding water, extraction was performed using methylene chloride (MC) and water. Thereafter, water was removed from the extract with magnesium sulfate (MgSO₄), and then the water-removed extract was separated by a silica gel column to obtain 29.2 g of Compound 300-1-1 (yield 92%).

The compounds were synthesized in the same manner as in Preparation Example 5 above by using Intermediate A of Table 5 below instead of di([1,1′-biphenyl]-4-yl)amine (A) and using Intermediate B of Table 5 below instead of 10-chloro-2,7-diphenyltribenzo[b,d,f]oxepine (B) in Preparation Example 5 above.

TABLE 5

| Compound number | Intermediate A | Intermediate B | Yield |
|---|---|---|---|
| 986 | 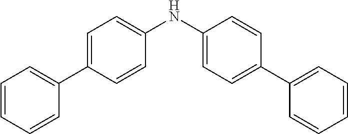 [SMILES: c1ccc(-c2ccc(Nc3ccc(-c4ccccc4)cc3)cc2)cc1] | 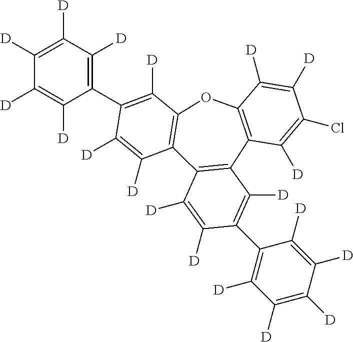 [SMILES: [2H]c1c([2H])c([2H])c(-c2c([2H])c([2H])c3c(c2[2H])Oc2c([2H])c([2H])c(Cl)c([2H])c2-c2c([2H])c(-c4c([2H])c([2H])c([2H])c([2H])c4[2H])c([2H])c([2H])c2-3)c([2H])c1[2H]] | 70% |
| 471 | 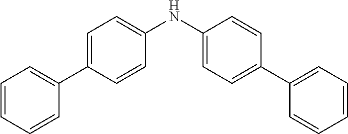 [SMILES: c1ccc(-c2ccc(Nc3ccc(-c4ccccc4)cc3)cc2)cc1] | 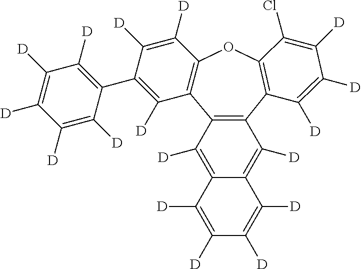 [SMILES: [2H]c1c([2H])c([2H])c(-c2c([2H])c([2H])c3c(c2[2H])-c2c(c([2H])c4c([2H])c([2H])c([2H])c([2H])c4c2[2H])-c2c([2H])c([2H])c([2H])c(Cl)c2O3)c([2H])c1[2H]] | 69% |
| 615 | 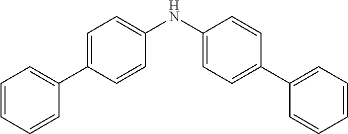 [SMILES: c1ccc(-c2ccc(Nc3ccc(-c4ccccc4)cc3)cc2)cc1] | 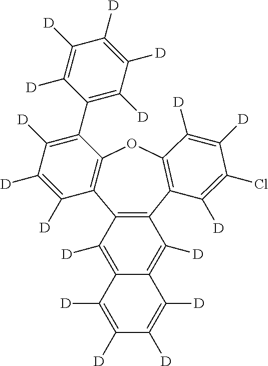 [SMILES: [2H]c1c([2H])c([2H])c(-c2c([2H])c([2H])c([2H])c3c2Oc2c([2H])c([2H])c(Cl)c([2H])c2-c2c-3c([2H])c3c([2H])c([2H])c([2H])c([2H])c3c2[2H])c([2H])c1[2H]] | 68% |
| 759 | 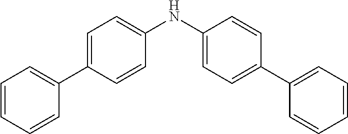 [SMILES: c1ccc(-c2ccc(Nc3ccc(-c4ccccc4)cc3)cc2)cc1] | 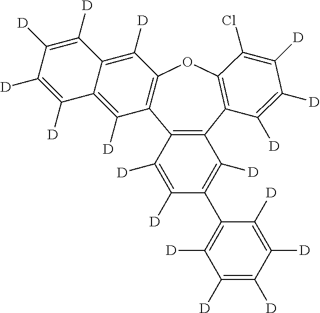 [SMILES: [2H]c1c([2H])c([2H])c(-c2c([2H])c([2H])c3c(c2[2H])-c2c([2H])c([2H])c([2H])c(Cl)c2Oc2c-3c([2H])c3c([2H])c([2H])c([2H])c([2H])c3c2[2H])c([2H])c1[2H]] | 71% |
| 903 | 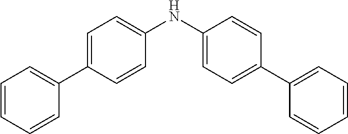 [SMILES: c1ccc(-c2ccc(Nc3ccc(-c4ccccc4)cc3)cc2)cc1] | 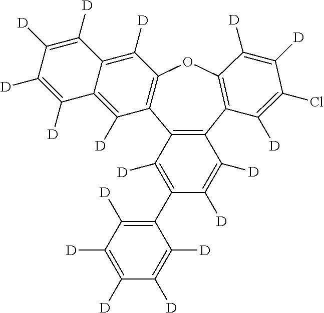 [SMILES: [2H]c1c([2H])c([2H])c(-c2c([2H])c([2H])c3c(c2[2H])-c2c(c([2H])c4c([2H])c([2H])c([2H])c([2H])c4c2[2H])Oc2c([2H])c([2H])c(Cl)c([2H])c2-3)c([2H])c1[2H]] | 70% |

### Preparation Example 6: Preparation of Compound 829

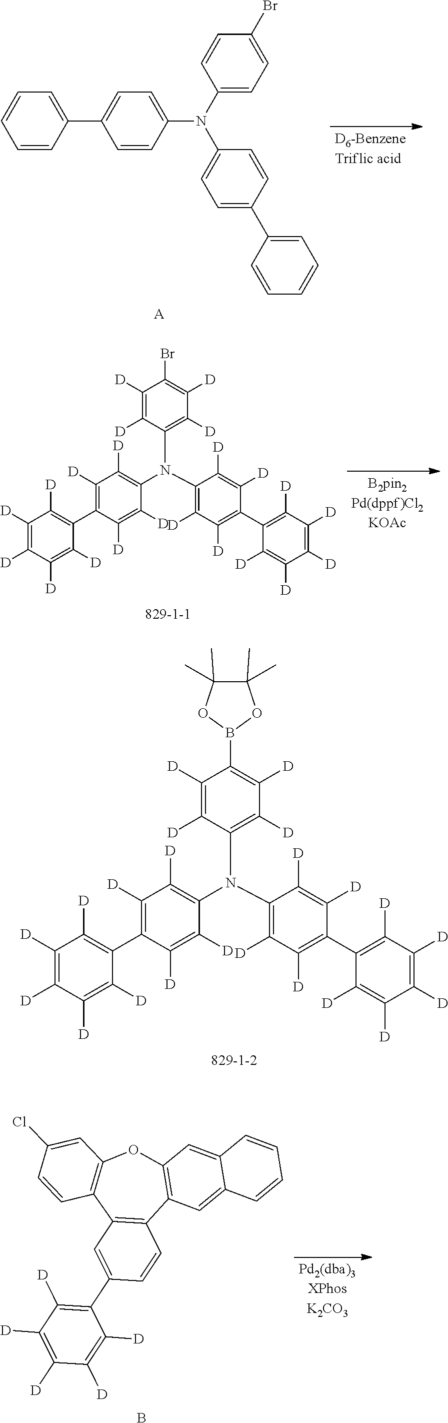

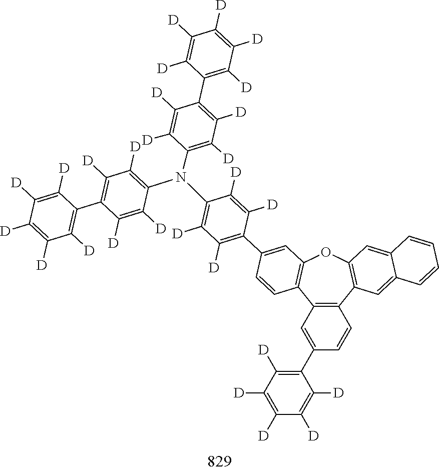 [SMILES: [2H]c1c([2H])c([2H])c(-c2ccc3c(c2)-c2ccc(-c4c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c4[2H])cc2Oc2cc4ccccc4cc2-3)c([2H])c1[2H]]

Compound 829-1-1 in Preparation Example 6 was synthesized in the same manner as in the preparation of Compound 300-1-1 of Preparation Example 5 above, Compound B is a compound synthesized by Preparation Example above, and Compound 829 was synthesized in the same manner as in the preparation of Compound 16 of Preparation Example 2 above.

### 1) Preparation of Compound 829-1-2

N-([1,1′-biphenyl]-4-yl-d9)-N-(4-bromophenyl-2,3,5,6-d4)-[1,1′-biphenyl]-4-amine-d9 (30 g, 0.060 mol, 1 eq) (A′), bis(pinacolato)diboron (B₂pin₂) (22.9 g, 0.090 mol, 1.5 eq), [1,1′-bis(diphenylphosphino)ferrocene]palladium (II) dichloride (Pd(dppf)Cl₂) (2.2 g, 0.003 mol, 0.05 eq), and potassium acetate (KOAc) (17.7 g, 0.180 mol, 3.0 eq) were added to 1,4-dioxane (300 ml), and stirred at 100° C. for 12 hours. After completion of the reaction by adding water, extraction was performed using methylene chloride (MC) and water. Thereafter, water was removed from the extract with magnesium sulfate (MgSO₄), and then the water-removed extract was separated by a silica gel column to obtain 27.8 g of Compound 829-1-2 (yield 85%).

The compounds were synthesized in the same manner as in Preparation Example 6 above by using Intermediate A of Table 6 below instead of N-([1,1′-biphenyl]-4-yl)-N-(4-bromophenyl)-[1,1′-biphenyl]-4-amine (A), which is an intermediate of N-([1,1′-biphenyl]-4-yl-d9)-N-(4-bromophenyl-2,3,5,6-d4)-[1,1′-biphenyl]-4-amine-d9 (A′), and using Intermediate B of Table 6 below instead of 7-chloro-3-(phenyl-d5)dibenzo[b,d]naphtho[2,3-f]oxepine (B) in Preparation Example 6 above.

TABLE 6

| Compound number | Intermediate A | Intermediate B | Yield |
|---|---|---|---|
| 829 | 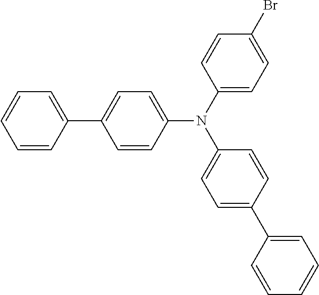 [SMILES: Brc1ccc(N(c2ccc(-c3ccccc3)cc2)c2ccc(-c3ccccc3)cc2)cc1] | 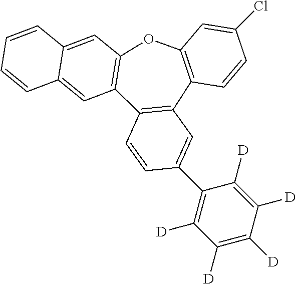 [SMILES: [2H]c1c([2H])c([2H])c(-c2ccc3c(c2)-c2ccc(Cl)cc2Oc2cc4ccccc4cc2-3)c([2H])c1[2H]] | 68% |
| 757 | 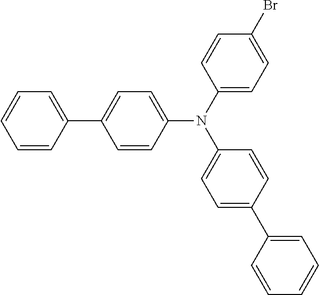 [SMILES: Brc1ccc(N(c2ccc(-c3ccccc3)cc2)c2ccc(-c3ccccc3)cc2)cc1] | 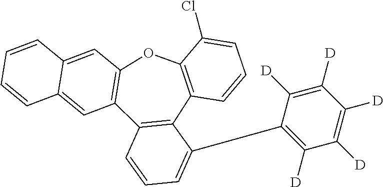 [SMILES: [2H]c1c([2H])c([2H])c(-c2cccc3c2-c2cccc(Cl)c2Oc2cc4ccccc4cc2-3)c([2H])c1[2H]] | 66% |

The remaining compounds other than the compounds described in Preparation Examples 1 to 6 and Tables 1 to 6 above were also prepared in the same manner as in the aforementioned Preparation Examples, and the synthesis results are shown in Tables 7 and 8. Table 7 below is the measurement values of 1H NMR (CDCl₃, 200 Mz), and Table 8 below is the measurement values of Field desorption mass spectrometry (FD-MS).

TABLE 7

| Compound | ¹H NMR (CDCl₃, 200 MHz) |
|---|---|
| 1 | δ = 8.05(2H, d), 7.96-7.86(4H, m), 7.75(2H, d), 7.61-7.55(8H, m), 7.49-7.41(7H, |
| | m), 7.37-7.24(7H, m), 7.16(1H, d), 1.69(6H, s) |
| 16 | δ = 8.05(4H, d), 7.96(2H, d), 7.75(4H, d), 7.60-7.51(10H, m), 7.49-7.37(17H, m) |
| 23 | δ = 8.15-8.13(2H, m), 8.05(4H, d), 7.90-7.86(2H, m), 7.75(4H, d), 7.55-7.41(18H, |
| | m), 7.38-7.28(8H, m), 7.16(1H, d), 1.69(6H, s) |
| 120 | δ = 8.15-8.13(2H, m), 8.05(2H, d), 7.75-7.69(7H, m), 7.55-7.51(6H, m), 7.49- |
| | 7.41(13H, m), 7.37-7.35(5H, m), 7.09(1H, d), 7.05(1H, s) |
| 197 | δ = 7.96-7.86(5H, m), 7.75-7.69(3H, m), 7.60-7.52(6H, m), 7.49-7.41(3H, m), 7.37- |
| | 7.28(5H, m), 7.16(1H, d), 7.09(1H, d), 7.05(1H, s) 6.97(1H, d) 1.69(6H, s) |
| 199 | δ = 8.15-8.13(2H, m), 7.95(1H, d), 7.75-7.69(5H, m), 7.55-7.41(11H, m), 7.37- |
| | 7.35(5H, m), 7.19-7.18(2H, m), 7.09-7.05(2H, m) |
| 217 | δ = 8.05(2H, d), 7.96-7.86(5H, m), 7.77-7.75(3H, m), 7.60-7.51(9H, m), 7.49- |
| | 7.41(7H, m), 7.38-7.33(7H, m), 7.28-7.25(5H, m), 7.16(1H, d), 1.69(6H, s) |
| 228 | δ = 7.96-7.92(3H, m), 7.78-7.71(6H, m), 7.60-7.52(6H, m), 7.49-7.37(11H, m), 7.24- |
| | 7.23(2H, m), 7.15-7.11(2H, m), 6.97(1H, d) |
| 233 | δ = 8.08-7.86(8H, m), 7.75(2H, d), 7.60-7.51(8H, m), 7.49-7.41(3H, m), 7.39- |
| | 7.31(6H, m), 7.28-7.23(3H, m), 7.16-7.15(2H, m), 6.97(1H, d), 1.69(6H, s) |
| 246 | δ = 8.15-8.13(2H, m), 7.90-7.86(3H, m), 7.75(6H, d), 7.55-7.41(12H, m), 7.38- |
| | 7.33(3H, m), 7.28-7.23(8H, m), 7.18-7.15(4H, m), 6.97(1H, d) 1.69(6H, s) |
| 247 | δ = 7.96-7.92(3H, m), 7.75(4H, d), 7.60-7.52(5H, m), 7.49-7.37(8H, m), 7.25- |
| | 7.23(12H, m), 7.15-6.97(5H, m) |
| 300 | All D Substituents |
| 471 | All D Substituents |
| 508 | δ = 8.57(2H, s), 8.15(2H, d), 7.90(1H, s), 7.77-7.64(10H, m), 7.55-7.41(13H, m), |
| | 7.38-7.37(5H, m), 7.09-7.05(2H, m) |
| 514 | δ = 8.57(2H, s), 8.15(2H, d), 7.90-7.86(3H, m), 7.77-7.64(8H, m), 7.55-7.41(9H, |
| | m), 7.38-7.33(5H, m), 7.28-7.18(7H, m), 7.16-7.05(7H, m) |
| 522 | δ = 8.57(2H, s), 8.15(2H, d), 7.92-7.90(2H, m), 7.77-7.75(7H, m), 7.64(2H, d), |
| | 7.55-7.52(7H, m), 7.49-7.41(9H, m), 7.38-7.37(7H, m), 6.97(1H, d) |
| 541 | δ = 8.57(2H, s), 8.15(2H, d), 7.92-7.86(4H, m), 7.77-7.75(3H, m), 7.64(2H, d), |
| | 7.55-7.52(6H, m), 7.49-7.41(3H, m), 7.38-7.37(8H, m), 7.16(1H, d), 6.97(1H, d), |
| | 1.69(6H, s) |
| 562 | δ = 8.57(2H, s), 8.15(2H, d), 7.92-7.86(3H, m), 7.75(4H, d), 7.64(2H, m), 7.55- |
| | 7.41(10H, m), 7.38-7.33(4H, m), 7.28-7.15(5H, m), 6.97(1H, d), 1.69(6H, s) |
| 578 | δ = 8.57(2H, s), 8.15(2H, d), 7.92-7.86(4H, m), 7.77-7.75(5H, m), 7.64(2H, d), |
| | 7.55-7.52(6H, m), 7.49-7.41(6H, m), 7.38-7.25(12H, m), 7.16(1H, d), 6.97(1H, d), |
| | 1.69(6H, s) |
| 615 | All D Substituents |
| 702 | δ = 8.95(1H, d), 8.50(1H, d), 8.20(1H, d), 8.10-8.04(6H, m), 7.83-7.75(7H, m), |
| | 7.61-7.52(8H, m), 7.49-7.41(6H, m), 7.39-7.37(5H, m), 7.27-7.24(6H, m) |
| 708 | δ = 8.15-8.04(5H, m), 7.90-7.83(5H, m), 7.75(2H, d), 7.61-7.52(5H, m), 7.49- |
| | 7.41(3H, m), 7.38-7.24(9H, m), 7.16(2H, d), 1.69(12H, s) |
| 721 | δ = 8.15-8.04(7H, m), 7.83(1H, s), 7.75(6H, d), 7.61-7.49(14H, m), 7.45-7.35(11H, |
| | m) |
| 757 | δ = 8.10-8.04(7H, m), 7.83(2H, m), 7.61-7.52(2H, m), 7.45(1H, m) |
| 759 | All D Substituents |
| 828 | δ = 8.10-8.04(5H, m), 7.92-7.75(9H, m), 7.61-7.49(10H, m), 7.46-7.37(9H, m), 7.33- |
| | 7.28(6H, m), 7.16(1H, d), 6.97(1H, d), 1.69(6H, s) |
| 829 | δ = 8.15-8.04(5H, m), 7.92(1H, d), 7.83(1H, s), 7.61-7.52(3H, m), 7.35(1H, d), |
| | 6.97(1H, d) |
| 854 | δ = 8.98(1H, d), 8.84(1H, d), 8.15-8.04(6H, m), 7.90-7.83(4H, m), 7.75-7.61(7H, |
| | m), 7.55-7.41(6H, m), 7.38-7.23(6H, m), 7.16-7.15(2H, m), 1.69(6H, s) |
| 865 | δ = 8.15-8.04(5H, m), 7.90(1H, s), 7.83-7.75(8H, m), 7.61-7.49(14H, m), 7.41- |
| | 7.35(11H, m) |
| 903 | All D Substituents |
| 985 | δ = 8.15-8.13(2H, m), 7.95-7.86(3H, m), 7.75(4H, d), 7.61-7.49(8H, m), 7.45- |
| | 7.37(6H, m), 7.35-7.27(4H, m), 7.24-7.16(4H, m), 1.69(6H, s) |
| 986 | δ = 8.15-8.13(2H, m), 8.05(2H, d), 7.95(1H, d), 7.75(6H, d), 7.55-7.49(12H, m), |
| | 7.45-7.41(5H, m), 7.37-7.35(7H, m), 7.19-7.18(2H, m) |
| 989 | δ = 8.95(1H, d), 8.50(1H, d), 8.20-8.09(4H, m), 7.95-7.86(3H, m), 7.77-7.75(3H, |
| | m), 7.61-7.49(7H, m), 7.45-7.37(6H, m), 7.35-7.28(4H, m), 7.24-7.16(4H, m), |
| | 1.69(1H, s) |
| 1007 | δ = 8.06(2H, d), 7.95(1H, d), 7.83-7.69(8H, m), 7.55-7.46(10H, m), 7.45-7.37(8H, |
| | m), 7.19-7.18(2H, m), 7.09-7.05(2H, m) |
| 1010 | δ = 8.15-8.13(2H, m), 7.95-7.92(2H, m), 7.75(6H, d), 7.55-7.49(13H, m), 7.45- |
| | 7.35(11H, m), 7.19-7.18(2H, m), 6.97(1H, d) |
| 1011 | δ = 8.15-8.13(2H, m), 7.95(1H, d), 7.75-7.69(7H, m), 7.55-7.46(9H, m), 7.45- |
| | 7.41(4H, m), 7.37-7.27(4H, m), 7.19-7.17(4H, m), 7.09-7.05(2H, m) |
| 1012 | δ = 8.15-8.13(2H, m), 7.95-7.86(3H, m), 7.75-7.69(5H, m), 7.55-7.46(6H, m), 7.45- |
| | 7.41(3H, m), 7.38-7.27(5H, m), 7.19-7.16(5H, m), 7.09-7.05(2H, m), 1.69(6H, s) |
| 1015 | δ = 8.15-8.13(2H, m), 7.95-7.86(4H, m), 7.75(4H, d), 7.55-7.49(9H, m), 7.45- |
| | 7.38(4H, m), 7.37-7.27(6H, m), 7.19-7.16(5H, m), 6.97(1H, d), 1.69(6H, s) |
| 1046 | δ = 8.06(2H, d), 7.95(1H, d), 7.83-7.71(7H, m), 7.55-7.46(7H, m), 7.45-7.37(8H, |
| | m), 7.24-7.11(6H, m) |
| 1051 | δ = 8.06(2H, d), 7.95-7.75(10H, m), 7.55-7.49(7H, m), 7.46-7.37(11H, m), 7.33- |
| | 7.28(2H, m), 7.19-7.16(3H, m), 1.69(6H, s) |

TABLE 8

| Compound | FD-MS | Compound | FD-MS |
|---|---|---|---|
| 1 | m/z = 679.29 | 16 | m/z = 715.29 |
| | (C₅₁H₃₇NO = 679.86) | | (C₅₄H₃₇NO = 715.90) |
| 23 | m/z = 831.35 | 120 | m/z = 715.29 |
| | (C₆₃H₄₅NO = 832.06) | | (C₅₄H₃₇NO = 715.90) |
| 197 | m/z = 684.32 | 199 | m/z = 644.29 |
| | (C₅₁H₃₂D₅NO = 684.89) | | (C₄₈H₂₈D₅NO = 644.83) |
| 217 | m/z = 831.35 | 228 | m/z = 613.24 |
| | (C₆₃H₄₅NO = 832.06) | | (C₄₆H₃₁NO = 613.76) |
| 233 | m/z = 769.30 | 246 | m/z = 831.35 |
| | (C₅₇H₃₉NO₂ = 769.94) | | (C₆₃H₄₅NO = 832.06) |
| 247 | m/z = 715.29 | 300 | m/z = 752.52 |
| | (C₅₄H₃₇NO = 715.90) | | (C₅₄D₃₇NO = 753.12) |
| 471 | m/z = 724.49 | 508 | m/z = 689.27 |
| | (C₅₂D₃₅NO = 725.07) | | (C₅₂H₃₅NO = 689.86) |
| 514 | m/z = 853.33 | 522 | m/z = 765.30 |
| | (C₆₅H₄₃NO = 854.06) | | (C₅₈H₃₉NO = 765.96) |
| 541 | m/z = 810.37 | 562 | m/z = 729.30 |
| | (C₆₁H₃₈D₅NO = 811.05) | | (C₅₅H₃₉NO = 729.92) |
| 578 | m/z = 881.37 | 615 | m/z = 724.49 |
| | (C₆₇H₄₇NO = 882.12) | | (C₅₂D₃₅NO = 725.07) |
| 702 | m/z = 815.32 | 708 | m/z = 769.33 |
| | (C₆₂H₄₁NO = 816.02) | | (C₅₈H₄₃NO = 769.99) |
| 721 | m/z = 765.30 | 757 | m/z = 792.47 |
| | (C₅₈H₃₉NO = 765.96) | | (C₅₈H₁₂D₂₇NO = 793.12) |
| 759 | m/z = 724.49 | 828 | m/z = 881.37 |
| | (C₅₂D₃₅NO = 725.07) | | (C₆₇H₄₇NO = 882.12) |
| 829 | m/z = 792.47 | 854 | m/z = 753.30 |
| | (C₅₈H₁₂D₂₇NO = 793.12) | | (C₅₇H₃₉NO = 753.94) |
| 865 | m/z = 765.30 | 903 | m/z = 724.49 |
| | (C₅₈H₃₉NO = 765.96) | | (C₅₂D₃₅NO = 725.07) |
| 985 | m/z = 679.29 | 986 | m/z = 715.29 |
| | (C₅₁H₃₇NO = 679.86) | | (C₅₄H₃₇NO = 715.90) |
| 989 | m/z = 729.30 | 1007 | m/z = 639.26 |
| | (C₅₅H₃₉NO = 729.92) | | (C₄₈H₃₃NO = 639.80) |
| 1010 | m/z = 715.29 | 1011 | m/z = 639.26 |
| | (C₅₄H₃₇NO = 715.90) | | (C₄₈H₃₃NO = 639.80) |
| 1012 | m/z = 679.29 | 1015 | m/z = 755.32 |
| | (C₅₁H₃₇NO = 679.86) | | (C₅₇H₄₁NO = 755.96) |
| 1046 | m/z = 613.24 | 1051 | m/z = 755.32 |
| | (C₄₆H₃₁NO = 613.76) | | (C₅₇H₄₁NO = 755.96) |

### EXPERIMENTAL EXAMPLE

### Experimental Example 1

### (1) Manufacture of Organic Light Emitting Devices

After a transparent electrode Indium Tin Oxide (ITO) thin film obtained from glass for OLED (manufactured by Samsung-Corning) was ultrasonically washed for 5 minutes each using trichloroethylene, acetone, ethanol, and distilled water sequentially, the ultrasonically washed transparent electrode ITO thin film was put into isopropanol and stored, and then used. Next, an ITO substrate was installed in a substrate folder of vacuum deposition equipment, and the following 4,4′,4″-tris(N,N-(2-naphthyl)-phenylamino)triphenylamine (2-TNATA) was put into a cell in the vacuum deposition equipment.

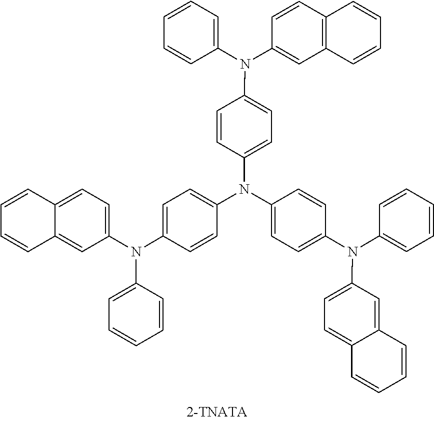 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, after evacuating the chamber until the vacuum degree in a chamber reached 10⁻⁶ torr, an electric current was applied to the cell to evaporate 2-TNATA, thereby depositing a hole injection layer of a thickness of 600 Å on the ITO substrate. The compounds shown in Table 9 below were put into another cell in the vacuum deposition equipment, and an electric current was applied to the cell to evaporate the compounds, thereby depositing a hole transport layer with a thickness of 300 Å on the hole injection layer.

After the hole injection layer and the hole transport layer were formed in this way, a blue light emitting material having the following structure was deposited thereon as a light emitting layer. Specifically, for the blue light emitting material, H1, a blue light emitting host material, was vacuum-deposited to a thickness of 200 Å on one cell in the vacuum deposition equipment, and D1, a blue light emitting dopant material, was vacuum-deposited thereon to a thickness of 5% compared to the host material.

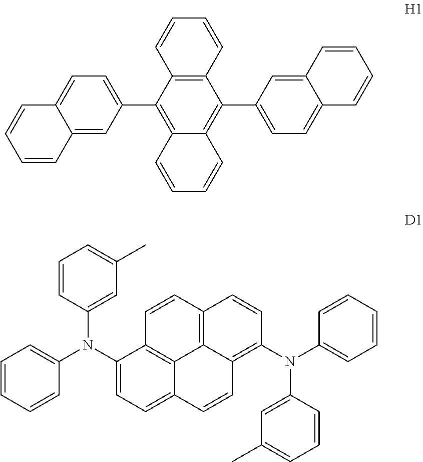

Subsequently, as an electron transport layer, a compound of the following structural formula E1 was deposited to a thickness of 300 Å.

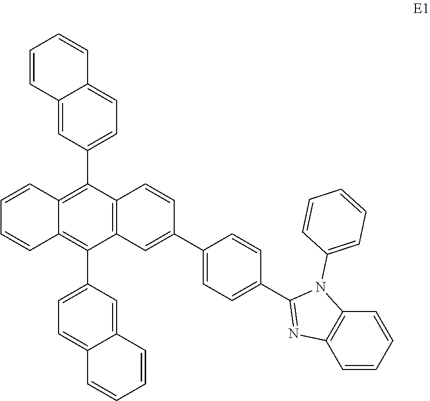 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

As an electron injection layer, lithium fluoride (LiF) was deposited to a thickness of 10 Å, and an aluminum (Al) negative electrode was deposited to a thickness of 1,000 Å to manufacture an organic light emitting device.

Meanwhile, all organic compounds required for the manufacture of the organic light emitting device were vacuum sublimated and purified under 10⁻⁶ to 10⁻⁸ torr for each material respectively, and used in the manufacture of the organic light emitting device.

### (2) Driving Voltages and Luminous Efficiencies of Organic Light Emitting Devices

For each of the organic light emitting devices of Examples 1 to 30 and Comparative Examples 1 to 7 manufactured as described above, electroluminescence (EL) properties were respectively measured with M7000 of McScience Inc., and lifetime T₉₅ values, which are the time at which they become 95% of the initial luminance, were measured with the measurement results when the reference luminance was 700 cd/m² through a lifetime measurement system (M6000) manufactured by McScience Inc.

The results of measuring the driving voltages, luminous efficiencies, color coordinates (CIE), and lifetimes of the blue organic light emitting devices manufactured according to the present disclosure are shown in Table 9 below.

TABLE 9

| | | Driving | Luminous | | |
|---|---|---|---|---|---|
| | | voltage | efficiency | CIE | Lifetime |
| | Compound | (V) | (cd/A) | (x, y) | (T₉₅) |
| Comparative | NPB | 5.54 | 6.05 | (0.134, 0.100) | 61 |
| Example 1 | | | | | |
| Comparative | Comparative | 5.44 | 6.13 | (0.135, 0.101) | 63 |
| Example 2 | compound A | | | | |
| Comparative | Comparative | 5.75 | 6.22 | (0.134, 0.101) | 68 |
| Example 3 | compound B | | | | |
| Comparative | Comparative | 5.41 | 6.23 | (0.132, 0.101) | 67 |
| Example 4 | compound C | | | | |
| Comparative | Comparative | 5.43 | 6.22 | (0.133, 0.100) | 70 |
| Example 5 | compound D | | | | |
| Comparative | Comparative | 5.44 | 6.21 | (0.133, 0.101) | 71 |
| Example 6 | compound E | | | | |
| Comparative | Comparative | 5.24 | 6.25 | (0.132, 0.101) | 73 |
| Example 7 | compound F | | | | |
| Example 1 | 1 | 4.86 | 6.95 | (0.134, 0.100) | 90 |
| Example 2 | 16 | 4.83 | 7.05 | (0.133, 0.101) | 95 |
| Example 3 | 120 | 4.58 | 7.34 | (0.134, 0.101) | 118 |
| Example 4 | 197 | 4.85 | 6.95 | (0.133, 0.100) | 105 |
| Example 5 | 199 | 4.84 | 7.03 | (0.132, 0.101) | 103 |
| Example 6 | 228 | 4.83 | 6.92 | (0.133, 0.100) | 92 |
| Example 7 | 233 | 4.84 | 6.95 | (0.134, 0.100) | 93 |
| Example 8 | 247 | 4.53 | 7.35 | (0.133, 0.102) | 116 |
| Example 9 | 300 | 4.55 | 7.31 | (0.134, 0.101) | 118 |
| Example 10 | 508 | 4.83 | 7.00 | (0.133, 0.100) | 91 |
| Example 11 | 522 | 4.85 | 6.90 | (0.132, 0.101) | 90 |
| Example 12 | 562 | 4.88 | 6.93 | (0.134, 0.100) | 92 |
| Example 13 | 615 | 4.82 | 6.95 | (0.132, 0.102) | 115 |
| Example 14 | 708 | 4.89 | 6.91 | (0.133, 0.100) | 92 |
| Example 15 | 721 | 4.81 | 6.88 | (0.133, 0.100) | 90 |
| Example 16 | 759 | 4.85 | 6.93 | (0.134, 0.101) | 114 |
| Example 17 | 829 | 4.82 | 6.98 | (0.133, 0.101) | 110 |
| Example 18 | 854 | 4.89 | 6.93 | (0.132, 0.101) | 91 |
| Example 19 | 865 | 4.91 | 6.98 | (0.132, 0.102) | 91 |
| Example 20 | 903 | 4.81 | 7.04 | (0.132, 0.100) | 113 |
| Example 21 | 985 | 4.84 | 6.89 | (0.133, 0.100) | 90 |
| Example 22 | 986 | 4.89 | 6.99 | (0.133, 0.101) | 91 |
| Example 23 | 989 | 4.84 | 6.97 | (0.134, 0.100) | 93 |
| Example 24 | 1007 | 4.87 | 6.96 | (0.133, 0.100) | 92 |
| Example 25 | 1010 | 4.81 | 6.88 | (0.132, 0.102) | 93 |
| Example 26 | 1011 | 4.83 | 6.93 | (0.134, 0.101) | 94 |
| Example 27 | 1012 | 4.82 | 6.95 | (0.134, 0.102) | 93 |
| Example 28 | 1015 | 4.94 | 7.03 | (0.133, 0.100) | 92 |
| Example 29 | 1046 | 4.89 | 6.97 | (0.133, 0.100) | 92 |
| Example 30 | 1051 | 4.85 | 6.92 | (0.134, 0.101) | 92 |

The compound of Comparative Example 1 above is as follows:

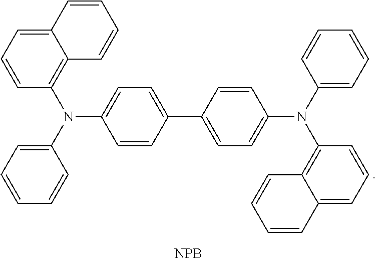 [SMILES: c1ccc(N(c2ccc(-c3ccc(N(c4ccccc4)c4cccc5ccccc45)cc3)cc2)c2cccc3ccccc23)cc1 ; "NPB"]

Further, the compounds of Comparative Examples 2 to 7 are the same as the following Comparative Compounds A to F:

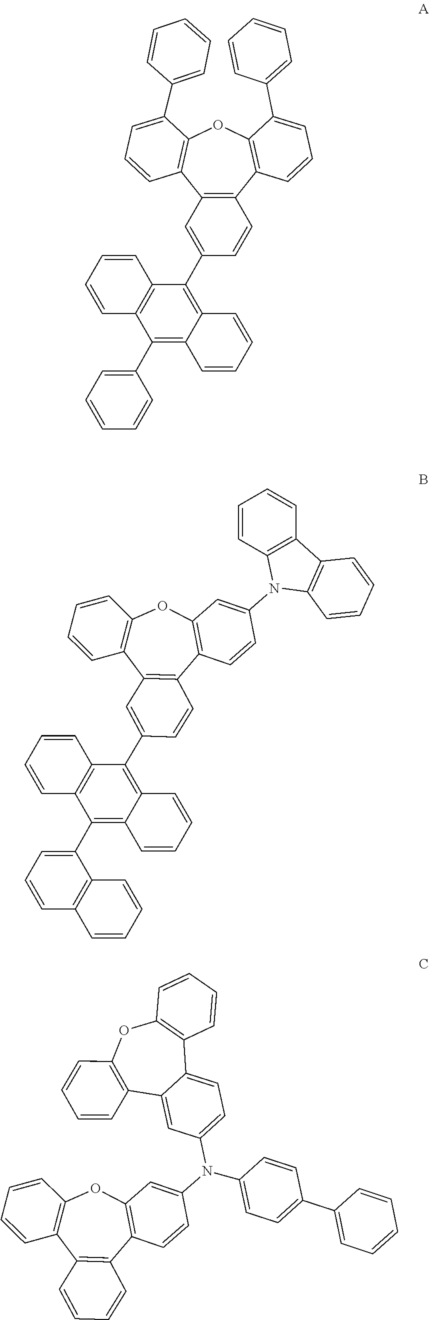 [SMILES: c1ccc(-c2cccc3c2Oc2c(-c4ccccc4)cccc2-c2cc(-c4c5ccccc5c(-c5ccccc5)c5ccccc45)ccc2-3)cc1 ; c1ccc2c(c1)Oc1cc(-n3c4ccccc4c4ccccc43)ccc1-c1ccc(-c3c4ccccc4c(-c4cccc5ccccc45)c4ccccc34)cc1-2 ; c1ccc(-c2ccc(N(c3ccc4c(c3)Oc3ccccc3-c3ccccc3-4)c3ccc4c(c3)-c3ccccc3Oc3ccccc3-4)cc2)cc1]

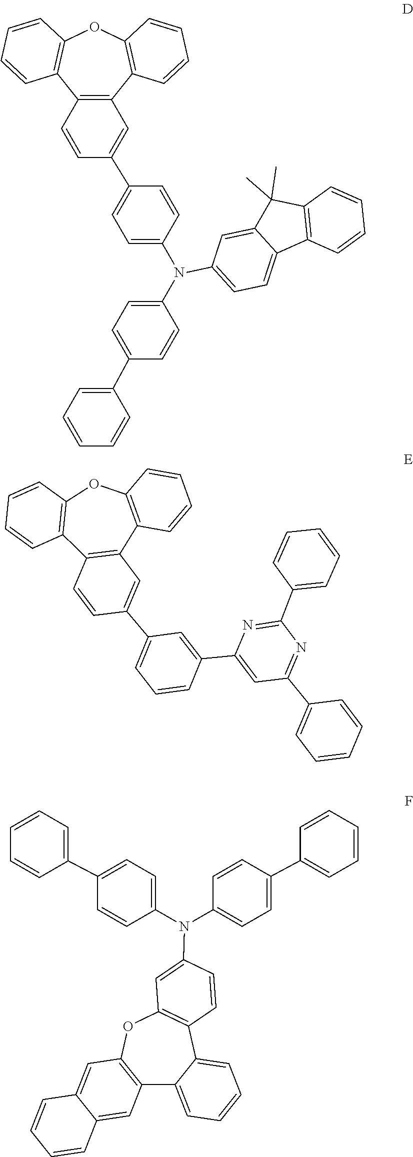

From the results of Table 9 above, it could be confirmed that driving voltages were low, and luminous efficiencies and lifetimes were remarkably improved in the blue organic light emitting devices of Examples 1 to 30 using the heterocyclic compound according to the present disclosure as the hole transport layer material compared to the organic light emitting devices using the compounds described in Comparative Examples 1 to 7. When the structures of the compounds described in Examples 1 to 30 above are compared with those of the compounds described in Comparative Examples 2 to 7, they are similar in that they have an oxepine backbone, but are different in that the substitution positions of the arylamine are different and they have additional substituents. Since the heterocyclic compound according to the present disclosure has a high triplet energy level (T₁ level) value due to these structural features, and enables the introduction of various substituents in the case of a tri-substituted oxepine derivative structure, it has advantages of securing high thermal stability of the material and enabling triplet energy levels to be adjusted. The heterocyclic compound according to the present disclosure using di- and tri-substituted oxepine derivatives has improved hole transport properties or improves stability compared to the compounds of Comparative Examples, thereby indicating that it is excellent in all aspects of driving voltage, luminous efficiency, and lifetime.

### Experimental Example 2

### (1) Manufacture of Organic Light Emitting Devices

After a transparent electrode Indium Tin Oxide (ITO) thin film obtained from glass for OLED (manufactured by Samsung-Corning) was ultrasonically washed for 5 minutes each using trichloroethylene, acetone, ethanol, and distilled water sequentially, the ultrasonically washed transparent electrode ITO thin film was put into isopropanol and stored, and then used. Next, an ITO substrate was installed in a substrate folder of vacuum deposition equipment, and 4,4′,4″-tris(N,N-(2-naphthyl)-phenylamino)triphenylamine (2-TNATA) below was put into a cell in the vacuum deposition equipment.

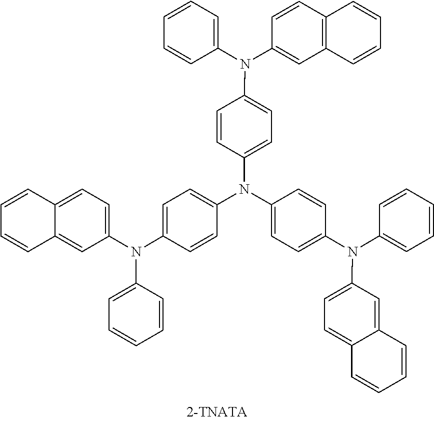 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, after evacuating the chamber until the vacuum degree in a chamber reached 10⁻⁶ torr, an electric current was applied to the cell to evaporate 2-TNATA, thereby depositing a hole injection layer with a thickness of 600 Å on the ITO substrate. N,N′-bis(α-naphthyl)-N,N′-diphenyl-4,4′-diamine (NPB) below was put into another cell in the vacuum deposition equipment, and an electric current was applied to the cell to evaporate NPB, thereby depositing a hole transport layer with a thickness of 250 Å on the hole injection layer. Thereafter, an electron blocking layer with a thickness of 50 Å was deposited on the hole transport layer by evaporating the compounds shown in Table 10.

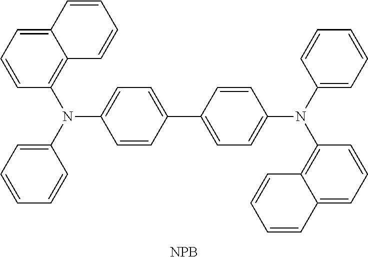 [SMILES: c1ccc(N(c2ccc(-c3ccc(N(c4ccccc4)c4cccc5ccccc45)cc3)cc2)c2cccc3ccccc23)cc1 ; "NPB"]

After the hole injection layer, the hole transport layer, and the electron blocking layer were formed in this way, a blue light emitting material having the following structure was deposited thereon as a light emitting layer. Specifically, for the blue light emitting material, H1, a blue light emitting host material, was vacuum-deposited to a thickness of 200 Å on one cell in the vacuum deposition equipment, and D1, a blue light emitting dopant material, was vacuum-deposited thereon to a thickness of 5% compared to the host material.

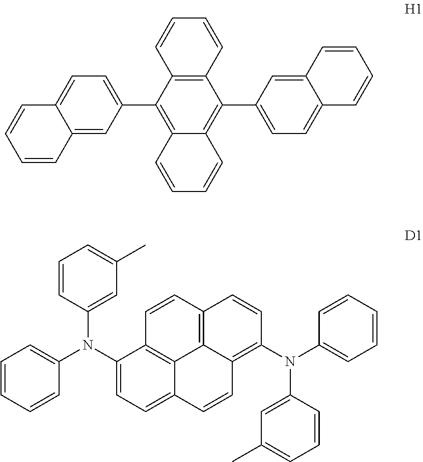

Subsequently, as an electron transport layer, a compound of the following structural formula E1 was deposited to a thickness of 300 Å.

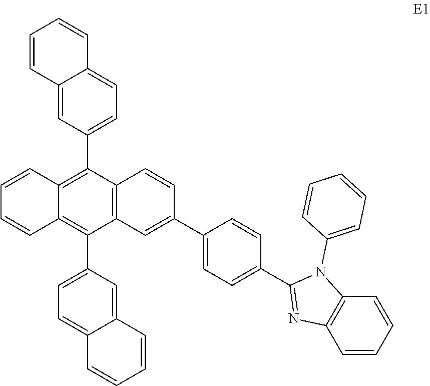 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

As an electron injection layer, lithium fluoride (LiF) was deposited to a thickness of 10 Å, and an aluminum (Al) negative electrode was deposited to a thickness of 1,000 Å to manufacture organic light emitting devices.

Meanwhile, all organic compounds required for the manufacture of the organic light emitting devices were vacuum sublimated and purified under 10⁻⁶ to 10⁻⁸ torr for each material respectively, and used in the manufacture of the organic light emitting devices.

### (2) Driving Voltages and Luminous Efficiencies of Organic Light Emitting Devices

For each of the organic light emitting devices of Examples 31 to 60 and Comparative Examples 8 to 14 manufactured as described above, electroluminescence (EL) properties were respectively measured with M7000 of McScience Inc., and lifetime T₉₅ values, which are the time at which they become 95% of the initial luminance, were measured with the measurement results when the reference luminance was 700 cd/m² through a lifetime measurement system (M6000) manufactured by McScience Inc.

The results of measuring the driving voltages, luminous efficiencies, color coordinates (CIE), and lifetimes of the blue organic light emitting devices manufactured according to the present disclosure are shown in Table 10 below.

TABLE 10

| | | Driving | Luminous | | |
|---|---|---|---|---|---|
| | | voltage | efficiency | CIE | Lifetime |
| | Compound | (V) | (cd/A) | (x, y) | (T₉₅) |
| Comparative | NPB | 5.55 | 6.07 | (0.134, 0.100) | 53 |
| Example 8 | | | | | |
| Comparative | Comparative | 5.42 | 6.13 | (0.134, 0.100) | 52 |
| Example 9 | Compound A | | | | |
| Comparative | Comparative | 5.33 | 6.11 | (0.133, 0.100) | 54 |
| Example 10 | Compound B | | | | |
| Comparative | Comparative | 5.31 | 6.18 | (0.133, 0.100) | 59 |
| Example 11 | Compound C | | | | |
| Comparative | Comparative | 5.32 | 6.20 | (0.134, 0.100) | 60 |
| Example 12 | Compound D | | | | |
| Comparative | Comparative | 5.32 | 6.19 | (0.134, 0.101) | 58 |
| Example 13 | Compound E | | | | |
| Comparative | Comparative | 5.34 | 6.23 | (0.134, 0.100) | 61 |
| Example 14 | Compound F | | | | |
| Example 31 | 1 | 4.73 | 6.89 | (0.133, 0.100) | 90 |
| Example 32 | 16 | 4.70 | 6.85 | (0.133, 0.101) | 89 |
| Example 33 | 120 | 4.35 | 7.44 | (0.134, 0.100) | 109 |
| Example 34 | 197 | 4.70 | 6.90 | (0.134, 0.100) | 94 |
| Example 35 | 199 | 4.78 | 6.94 | (0.133, 0.101) | 89 |
| Example 36 | 228 | 4.80 | 7.01 | (0.134, 0.101) | 88 |
| Example 37 | 233 | 4.82 | 6.93 | (0.133, 0.100) | 90 |
| Example 38 | 247 | 4.31 | 7.34 | (0.134, 0.101) | 109 |
| Example 39 | 300 | 4.34 | 7.38 | (0.133, 0.101) | 110 |
| Example 40 | 508 | 4.78 | 6.88 | (0.134, 0.100) | 89 |
| Example 41 | 522 | 4.80 | 6.94 | (0.133, 0.100) | 90 |
| Example 42 | 562 | 4.70 | 6.79 | (0.134, 0.100) | 90 |
| Example 43 | 615 | 4.80 | 7.01 | (0.133, 0.100) | 110 |
| Example 44 | 708 | 4.78 | 6.94 | (0.133, 0.101) | 88 |
| Example 45 | 721 | 4.77 | 6.99 | (0.134, 0.101) | 87 |
| Example 46 | 759 | 4.83 | 7.03 | (0.134, 0.100) | 108 |
| Example 47 | 829 | 4.75 | 6.97 | (0.134, 0.100) | 99 |
| Example 48 | 854 | 4.76 | 7.00 | (0.133, 0.101) | 87 |
| Example 49 | 865 | 4.73 | 6.88 | (0.134, 0.100) | 87 |
| Example 50 | 903 | 4.73 | 6.89 | (0.133, 0.100) | 109 |
| Example 51 | 985 | 4.76 | 6.83 | (0.134, 0.100) | 86 |
| Example 52 | 986 | 4.78 | 6.90 | (0.133, 0.100) | 86 |
| Example 53 | 989 | 4.75 | 6.94 | (0.133, 0.100) | 86 |
| Example 54 | 1007 | 4.78 | 6.95 | (0.134, 0.100) | 88 |
| Example 55 | 1010 | 4.79 | 7.00 | (0.133, 0.101) | 86 |
| Example 56 | 1011 | 4.80 | 7.01 | (0.133, 0.100) | 87 |
| Example 57 | 1012 | 4.79 | 6.97 | (0.134, 0.100) | 89 |
| Example 58 | 1015 | 4.80 | 7.05 | (0.134, 0.100) | 90 |
| Example 59 | 1046 | 4.75 | 6.85 | (0.134, 0.100) | 86 |
| Example 60 | 1051 | 4.77 | 6.89 | (0.133, 0.100) | 87 |

The compound of Comparative Example 8 is as follows:

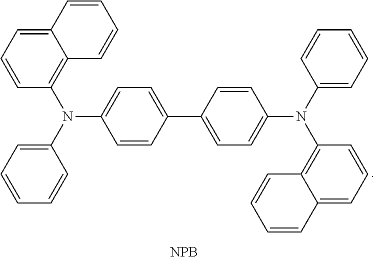 [SMILES: c1ccc(N(c2ccc(-c3ccc(N(c4ccccc4)c4cccc5ccccc45)cc3)cc2)c2cccc3ccccc23)cc1 ; "NPB"]

Further, the compounds of Comparative Examples 9 to 14 are the same as Comparative Compounds A to F described above.

From the results of Table 10 above, it could be confirmed that driving voltages were low, and luminous efficiencies and lifetimes were remarkably improved in the blue organic light emitting devices of Examples 31 to 60 using the heterocyclic compound according to the present disclosure as the electron blocking layer material compared to the organic light emitting devices using the compounds described in Comparative Examples 8 to 14.

When electrons move to the positive electrode by passing by the hole transport layer without being coupled in the light emitting layer, a phenomenon of reducing the efficiency and lifetime of the organic light emitting device occurs. In order to prevent such a phenomenon, when a compound having a high LUMO level and a triplet energy level (T₁ level) is used as the electron blocking layer, electrons that want to move to the positive electrode by passing by the light emitting layer will be blocked from moving by the energy barrier of the electron blocking layer. Due to this, the probability that holes and electrons form excitons in the light emitting layer increases, and the possibility that they are emitted into light from the light emitting layer increases. As these results, when the heterocyclic compound according to the present disclosure is used as a material for the electron blocking layer, the organic light emitting device is exhibited to be excellent in all aspects of driving voltage, efficiency, and lifetime.

Particularly, when the heterocyclic compound according to the present disclosure is used as a hole transport layer and an electron blocking layer, since the hole characteristics are strengthened, and the hole transport ability is improved and the stability of the molecule is also increased through adjustment of the band gap and triplet energy level (T₁ level) values, it can be confirmed that the driving voltage of the organic light emitting device is lowered, the light efficiency is improved, and the lifetime properties of the organic light emitting devices are improved by the improved thermal stability of the compound.

All simple modifications or changes of the present disclosure shall fall within the scope of the present disclosure, and the specific protection scope of the present disclosure will become clear from the appended claims.

1. A heterocyclic compound represented by the following Chemical Formula 1: 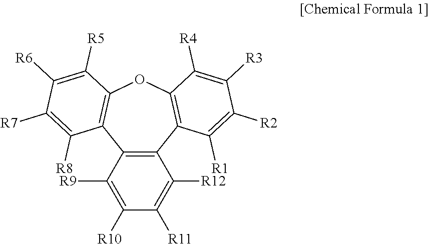
wherein,
R1 to R12 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and a group represented by Chemical Formula 2 below, or two or more groups adjacent to each other bond to each other to form a substituted or unsubstituted C6 to C60 aromatic hydrocarbon ring or a substituted or unsubstituted C2 to C60 heterocycle, and wherein A101, A102, and A103 are the same as or different from each other and are each independently a substituted or unsubstituted C1 to C60 alkyl group; a substituted or unsubstituted C6 to C60 aryl group; or a substituted or unsubstituted C2 to C60 heteroaryl group, 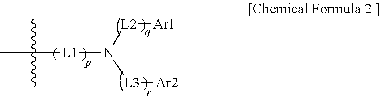
wherein,
L1, L2, and L3 are the same as or different from each other, and each independently a single bond; a substituted or unsubstituted C6 to C60 arylene group; or a substituted or unsubstituted C2 to C60 heteroarylene group,
Ar1 and Ar2 are the same as or different from each other, and are each independently a substituted or unsubstituted C6 to C60 aryl group; or a substituted or unsubstituted C2 to C60 heteroaryl group, and
p, q, and r are the same as or different from each other, and are each independently integers of 0 to 3.

2. The heterocyclic compound according to claim 1, wherein at least one of R1 to R8 is a group represented by Chemical Formula 2 above.

3. The heterocyclic compound according to claim 2, wherein, when at least one of R1 to R4 is a group represented by Chemical Formula 2 above, at least one of R5 to R12 is a substituted or unsubstituted C6 to C60 aryl group.

4. The heterocyclic compound according to claim 2, wherein, when at least one of R1 to R4 is a group represented by Chemical Formula 2 above, at least one of R5 to R8 is a substituted or unsubstituted C6 to C60 aryl group, and at least one of R9 to R12 is a substituted or unsubstituted C6 to C60 aryl group.

5. The heterocyclic compound according to claim 1, wherein the heterocyclic compound represented by Chemical Formula 1 above is represented by any one of the following Chemical Formula 1-1, Chemical Formula 1-2, and Chemical Formula 1-3: 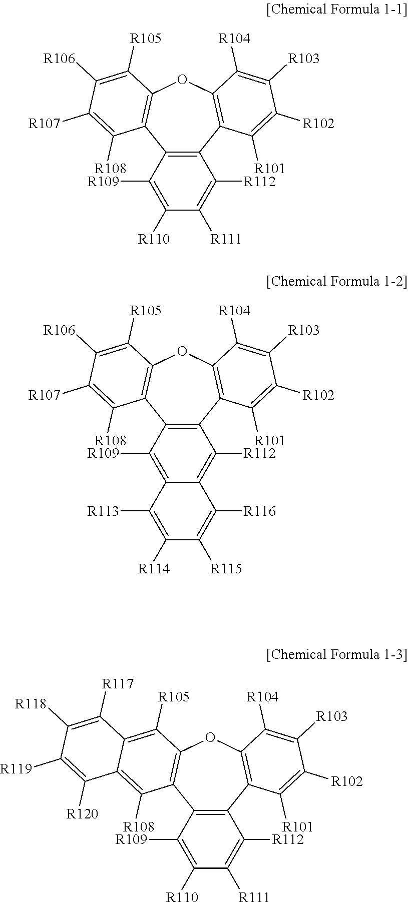
wherein,
R101 to R120 are the same as or different from each other, and are each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)A101A102; —SiA101A102A103; and a group represented by Chemical Formula 2 below, and wherein A101, A102 and A103 are the same as or different from each other and are each independently a substituted or unsubstituted C1 to C60 alkyl group; a substituted or unsubstituted C6 to C60 aryl group; or a substituted or unsubstituted C2 to C60 heteroaryl group, 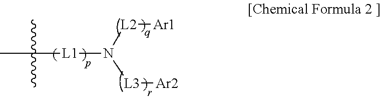
wherein,
L1, L2, L3, Ar1, Ar2, p, q and r are the same as defined above,
in Chemical Formula 1-1 above, when at least one of R101 to R104 is a group represented by Chemical Formula 2 above, at least one of R105 to R112 is a substituted or unsubstituted C6 to C60 aryl group,
in Chemical Formula 1-2 above, when at least one of R101 to R104 is a group represented by Chemical Formula 2 above, at least one of R105 to R109 and R112 to R116 is a substituted or unsubstituted C6 to C60 aryl group, and
in Chemical Formula 1-3 above, when at least one of R101 to R104 is a group represented by Chemical Formula 2 above, at least one of R105, R108 to R112, and R117 to R120 is a substituted or unsubstituted C6 to C60 aryl group.

6. The heterocyclic compound according to claim 5, wherein, in Chemical Formula 1-2 above, at least one of R105 to R108 is a substituted or unsubstituted C6 to C60 aryl group.

7. The heterocyclic compound according to claim 5, wherein, in Chemical Formula 1-3 above, at least one of R109 to R112 is a substituted or unsubstituted C6 to C60 aryl group.

8. The heterocyclic compound according to claim 1, wherein the heterocyclic compound represented by Chemical Formula 1 above does not contain deuterium as a substituent, or the content of deuterium based on the total number of hydrogen atoms and deuterium atoms is 1% to 100%.

9. The heterocyclic compound according to claim 1, wherein Chemical Formula 1 above is represented by any one of the following compounds:

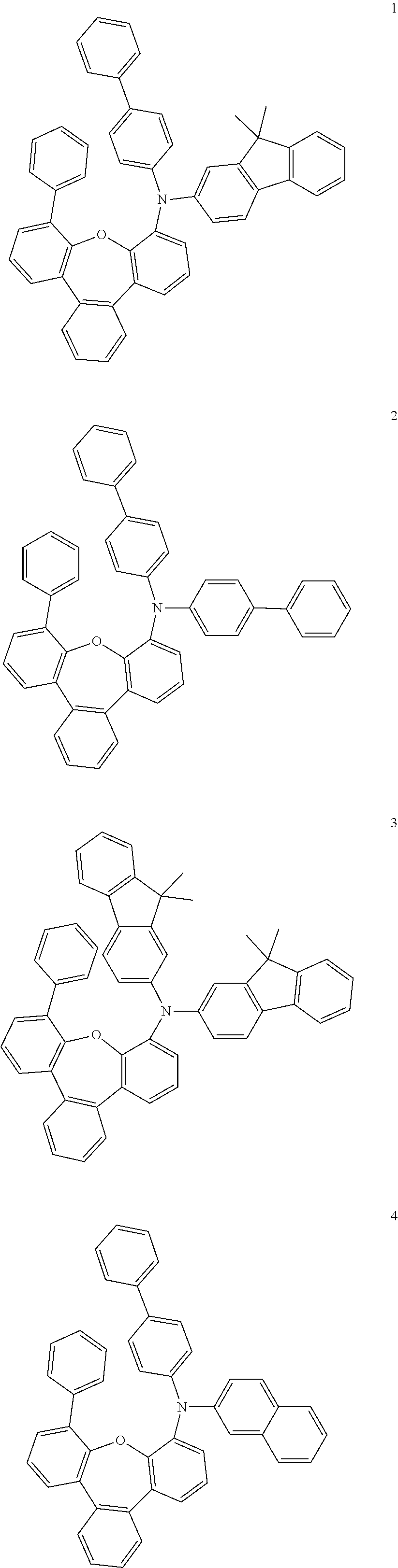 [SMILES: CC1(C)c2ccccc2-c2ccc(N(c3ccc(-c4ccccc4)cc3)c3cccc4c3Oc3c(-c5ccccc5)cccc3-c3ccccc3-4)cc21 ; c1ccc(-c2ccc(N(c3ccc(-c4ccccc4)cc3)c3cccc4c3Oc3c(-c5ccccc5)cccc3-c3ccccc3-4)cc2)cc1 ; CC1(C)c2ccccc2-c2ccc(N(c3ccc4c(c3)C(C)(C)c3ccccc3-4)c3cccc4c3Oc3c(-c5ccccc5)cccc3-c3ccccc3-4)cc21 ; c1ccc(-c2ccc(N(c3ccc4ccccc4c3)c3cccc4c3Oc3c(-c5ccccc5)cccc3-c3ccccc3-4)cc2)cc1]

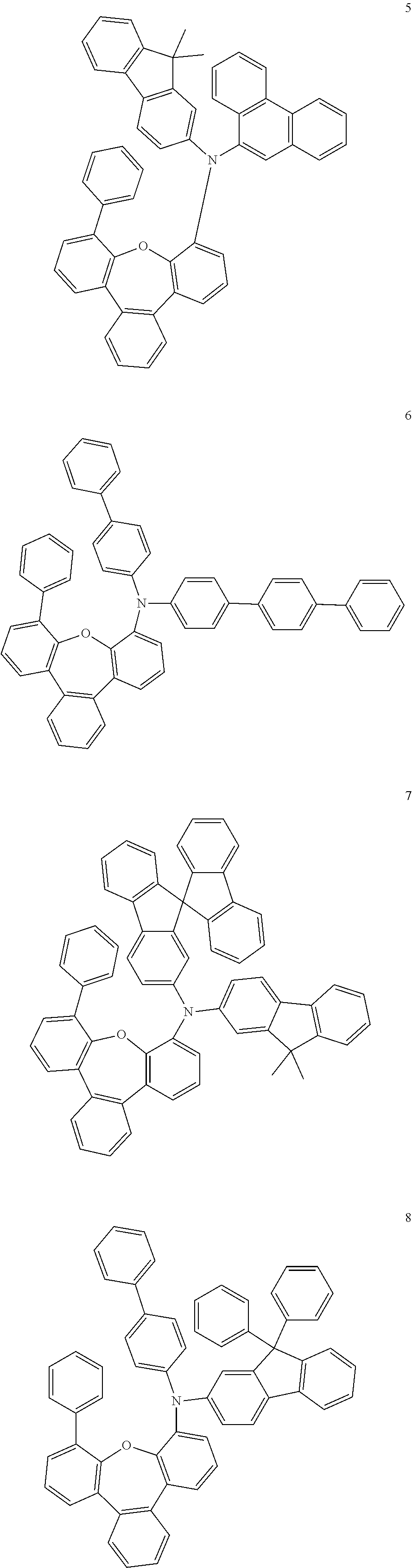 [SMILES: CC1(C)c2ccccc2-c2ccc(N(c3cccc4c3Oc3c(-c5ccccc5)cccc3-c3ccccc3-4)c3cc4ccccc4c4ccccc34)cc21 ; c1ccc(-c2ccc(-c3ccc(N(c4ccc(-c5ccccc5)cc4)c4cccc5c4Oc4c(-c6ccccc6)cccc4-c4ccccc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3c(-c5ccccc5)cccc3-c3ccccc3-4)cc21 ; c1ccc(-c2ccc(N(c3ccc4c(c3)C(c3ccccc3)(c3ccccc3)c3ccccc3-4)c3cccc4c3Oc3c(-c5ccccc5)cccc3-c3ccccc3-4)cc2)cc1]

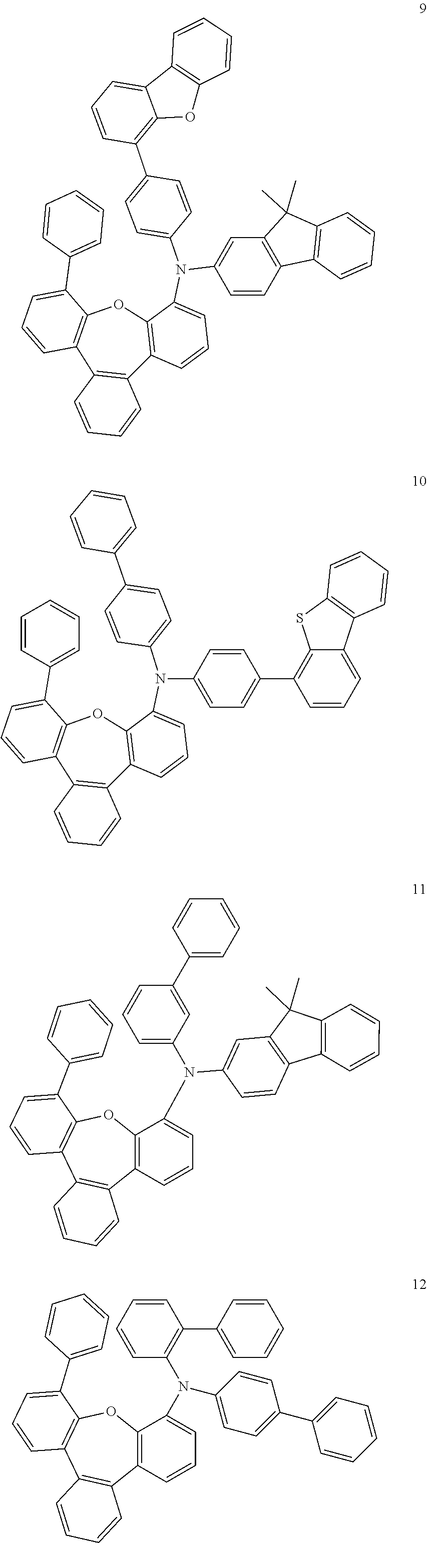 [SMILES: CC1(C)c2ccccc2-c2ccc(N(c3ccc(-c4cccc5c4oc4ccccc45)cc3)c3cccc4c3Oc3c(-c5ccccc5)cccc3-c3ccccc3-4)cc21 ; c1ccc(-c2ccc(N(c3ccc(-c4cccc5c4sc4ccccc45)cc3)c3cccc4c3Oc3c(-c5ccccc5)cccc3-c3ccccc3-4)cc2)cc1 ; CC1(C)c2ccccc2-c2ccc(N(c3cccc(-c4ccccc4)c3)c3cccc4c3Oc3c(-c5ccccc5)cccc3-c3ccccc3-4)cc21 ; c1ccc(-c2ccc(N(c3ccccc3-c3ccccc3)c3cccc4c3Oc3c(-c5ccccc5)cccc3-c3ccccc3-4)cc2)cc1]

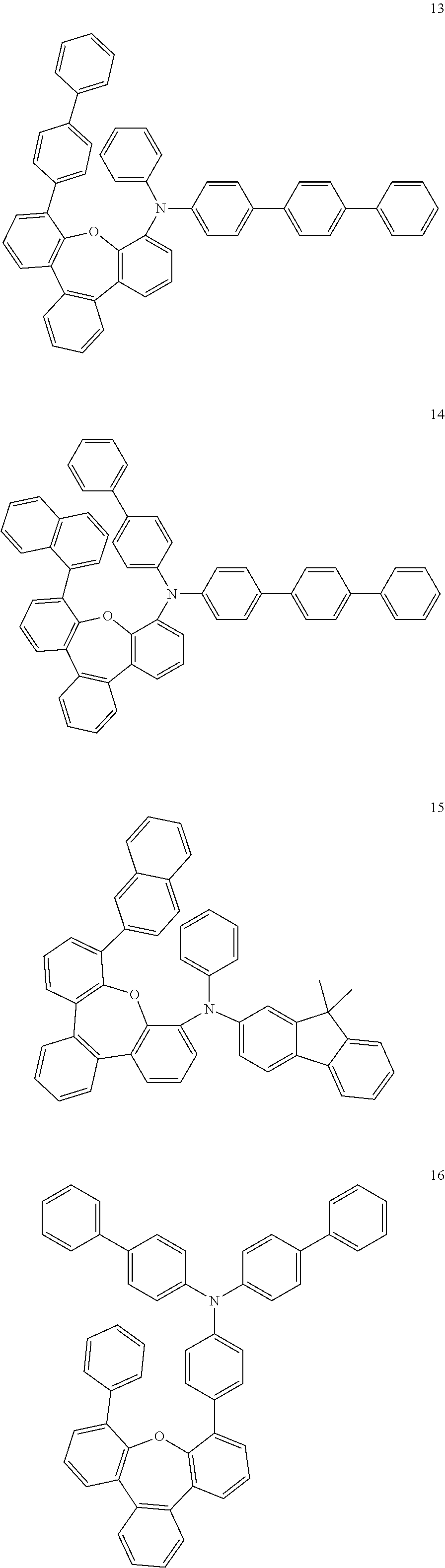 [SMILES: c1ccc(-c2ccc(-c3ccc(N(c4ccccc4)c4cccc5c4Oc4c(-c6ccc(-c7ccccc7)cc6)cccc4-c4ccccc4-5)cc3)cc2)cc1 ; c1ccc(-c2ccc(-c3ccc(N(c4ccc(-c5ccccc5)cc4)c4cccc5c4Oc4c(cccc4-c4cccc6ccccc46)-c4ccccc4-5)cc3)cc2)cc1 ; CC1(C)c2ccccc2-c2ccc(N(c3ccccc3)c3cccc4c3Oc3c(-c5ccc6ccccc6c5)cccc3-c3ccccc3-4)cc21 ; c1ccc(-c2ccc(N(c3ccc(-c4ccccc4)cc3)c3ccc(-c4cccc5c4Oc4c(-c6ccccc6)cccc4-c4ccccc4-5)cc3)cc2)cc1]

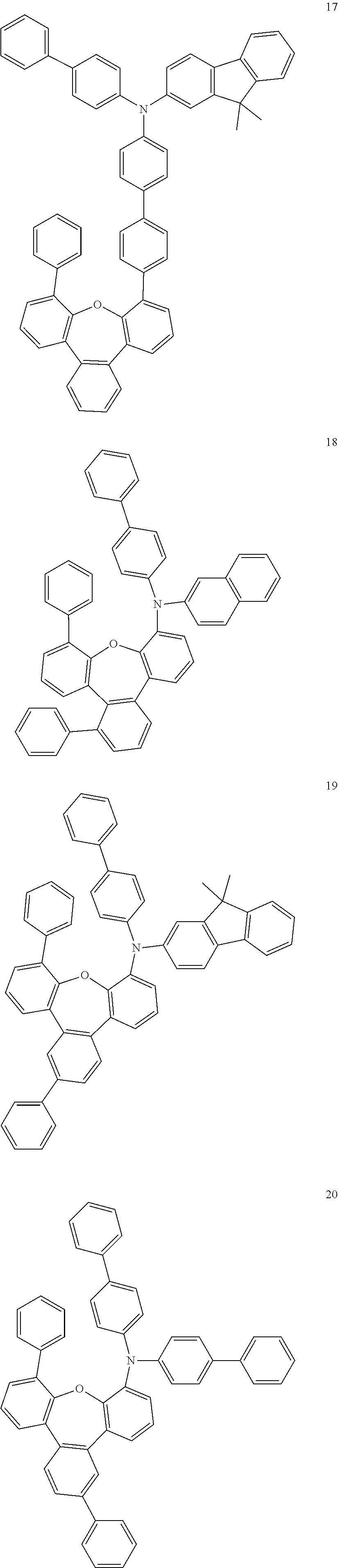 [SMILES: CC1(C)c2ccccc2-c2ccc(N(c3ccc(-c4ccccc4)cc3)c3ccc(-c4ccc(-c5cccc6c5Oc5c(-c7ccccc7)cccc5-c5ccccc5-6)cc4)cc3)cc21 ; c1ccc(-c2ccc(N(c3ccc4ccccc4c3)c3cccc4c3Oc3c(-c5ccccc5)cccc3-c3c(-c5ccccc5)cccc3-4)cc2)cc1 ; CC1(C)c2ccccc2-c2ccc(N(c3ccc(-c4ccccc4)cc3)c3cccc4c3Oc3c(-c5ccccc5)cccc3-c3cc(-c5ccccc5)ccc3-4)cc21 ; c1ccc(-c2ccc(N(c3ccc(-c4ccccc4)cc3)c3cccc4c3Oc3c(-c5ccccc5)cccc3-c3ccc(-c5ccccc5)cc3-4)cc2)cc1]

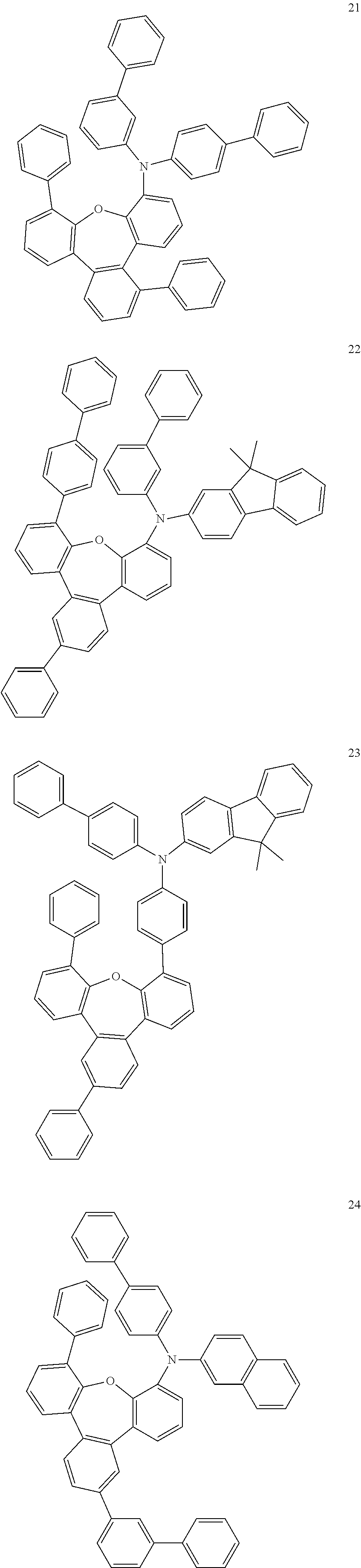

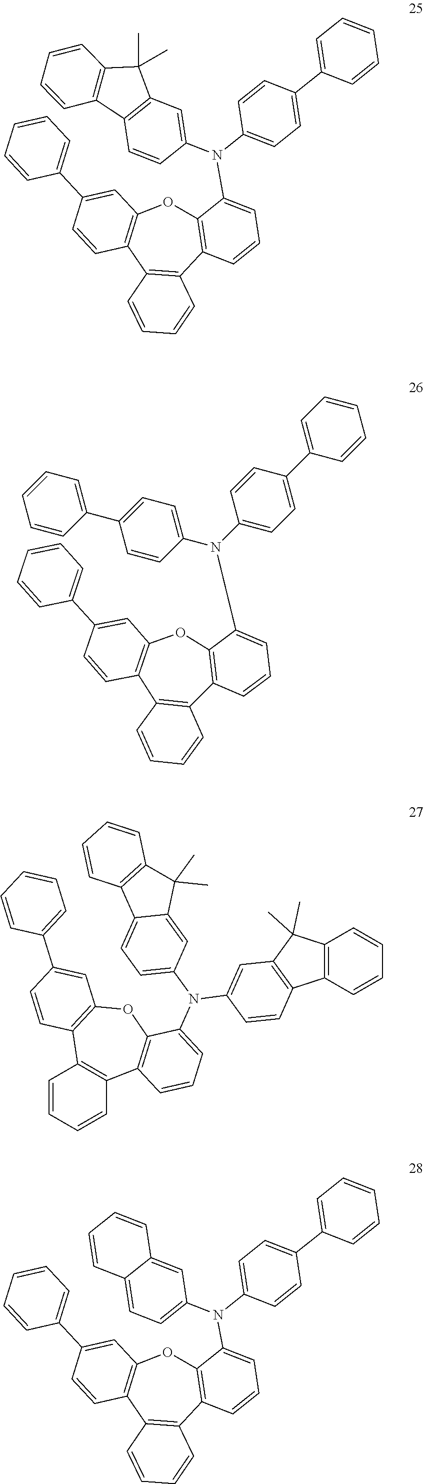 [SMILES: CC1(C)c2ccccc2-c2ccc(N(c3ccc(-c4ccccc4)cc3)c3cccc4c3Oc3cc(-c5ccccc5)ccc3-c3ccccc3-4)cc21 ; c1ccc(-c2ccc(N(c3ccc(-c4ccccc4)cc3)c3cccc4c3Oc3cc(-c5ccccc5)ccc3-c3ccccc3-4)cc2)cc1 ; CC1(C)c2ccccc2-c2ccc(N(c3ccc4c(c3)C(C)(C)c3ccccc3-4)c3cccc4c3Oc3cc(-c5ccccc5)ccc3-c3ccccc3-4)cc21 ; c1ccc(-c2ccc(N(c3ccc4ccccc4c3)c3cccc4c3Oc3cc(-c5ccccc5)ccc3-c3ccccc3-4)cc2)cc1]

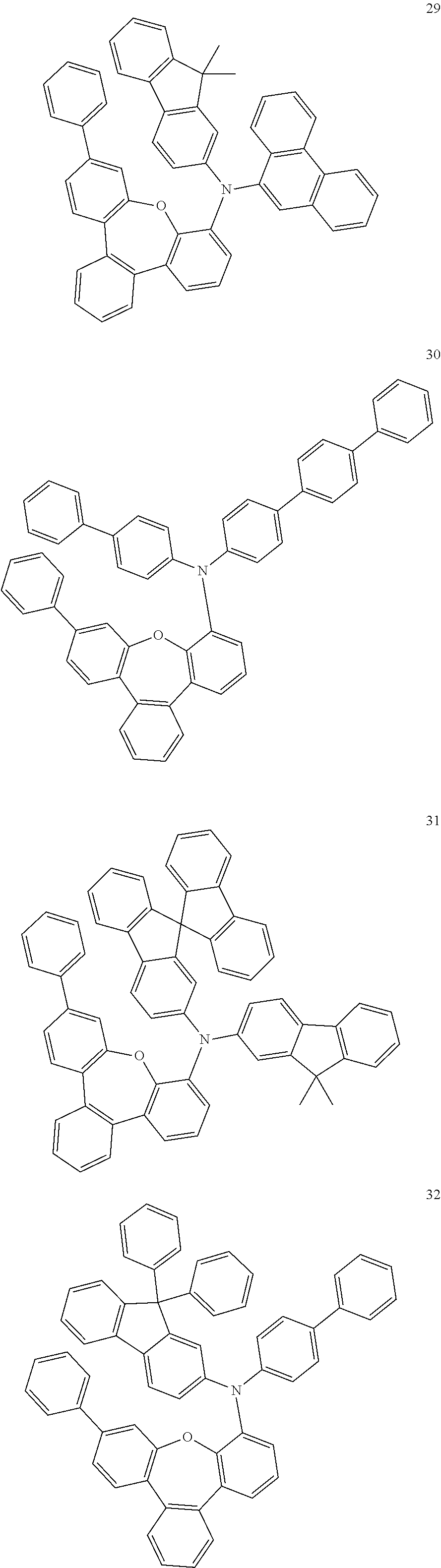 [SMILES: CC1(C)c2ccccc2-c2ccc(N(c3cccc4c3Oc3cc(-c5ccccc5)ccc3-c3ccccc3-4)c3cc4ccccc4c4ccccc34)cc21 ; c1ccc(-c2ccc(-c3ccc(N(c4ccc(-c5ccccc5)cc4)c4cccc5c4Oc4cc(-c6ccccc6)ccc4-c4ccccc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cc(-c5ccccc5)ccc3-c3ccccc3-4)cc21 ; c1ccc(-c2ccc(N(c3ccc4c(c3)C(c3ccccc3)(c3ccccc3)c3ccccc3-4)c3cccc4c3Oc3cc(-c5ccccc5)ccc3-c3ccccc3-4)cc2)cc1]

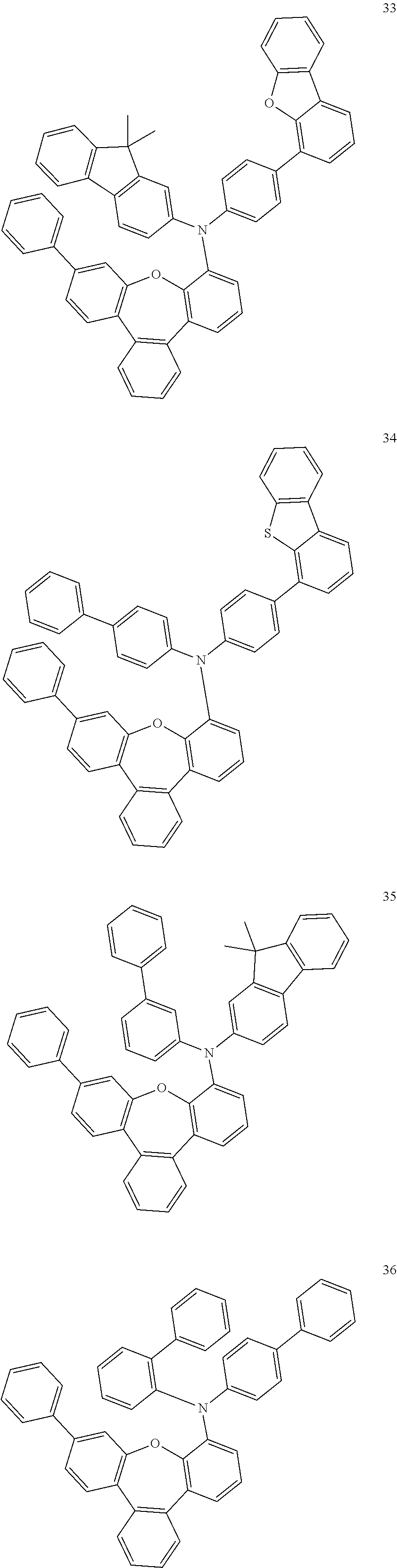 [SMILES: CC1(C)c2ccccc2-c2ccc(N(c3ccc(-c4cccc5c4oc4ccccc45)cc3)c3cccc4c3Oc3cc(-c5ccccc5)ccc3-c3ccccc3-4)cc21 ; c1ccc(-c2ccc(N(c3ccc(-c4cccc5c4sc4ccccc45)cc3)c3cccc4c3Oc3cc(-c5ccccc5)ccc3-c3ccccc3-4)cc2)cc1 ; CC1(C)c2ccccc2-c2ccc(N(c3cccc(-c4ccccc4)c3)c3cccc4c3Oc3cc(-c5ccccc5)ccc3-c3ccccc3-4)cc21 ; c1ccc(-c2ccc(N(c3ccccc3-c3ccccc3)c3cccc4c3Oc3cc(-c5ccccc5)ccc3-c3ccccc3-4)cc2)cc1]

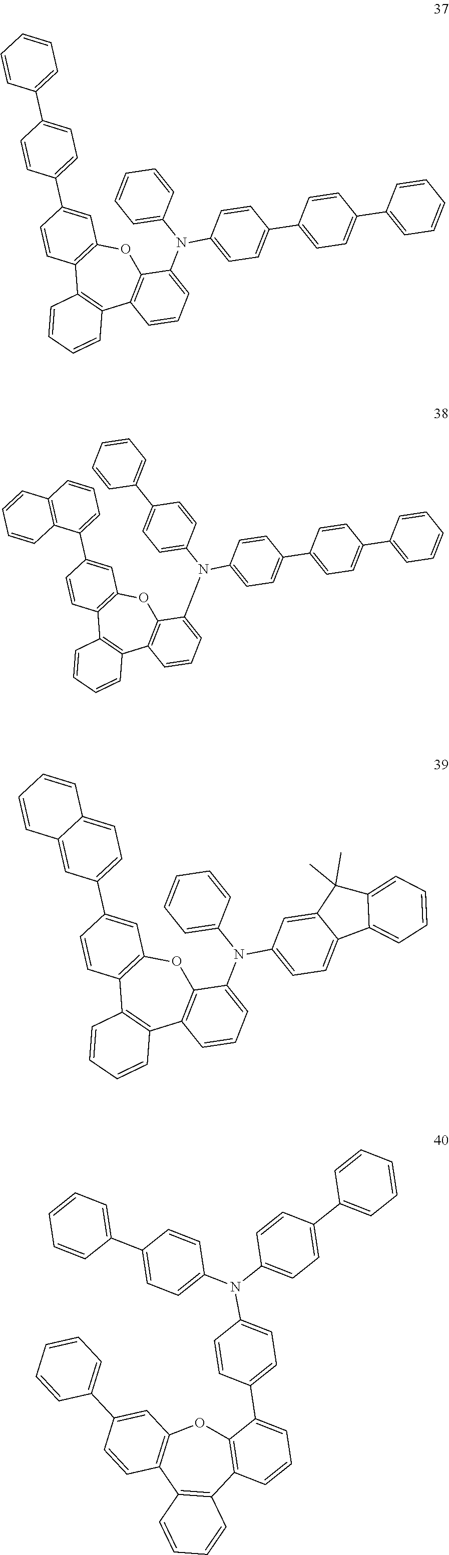 [SMILES: c1ccc(-c2ccc(-c3ccc(N(c4ccccc4)c4cccc5c4Oc4cc(-c6ccc(-c7ccccc7)cc6)ccc4-c4ccccc4-5)cc3)cc2)cc1 ; c1ccc(-c2ccc(-c3ccc(N(c4ccc(-c5ccccc5)cc4)c4cccc5c4Oc4cc(-c6cccc7ccccc67)ccc4-c4ccccc4-5)cc3)cc2)cc1 ; CC1(C)c2ccccc2-c2ccc(N(c3ccccc3)c3cccc4c3Oc3cc(-c5ccc6ccccc6c5)ccc3-c3ccccc3-4)cc21 ; c1ccc(-c2ccc(N(c3ccc(-c4ccccc4)cc3)c3ccc(-c4cccc5c4Oc4cc(-c6ccccc6)ccc4-c4ccccc4-5)cc3)cc2)cc1]

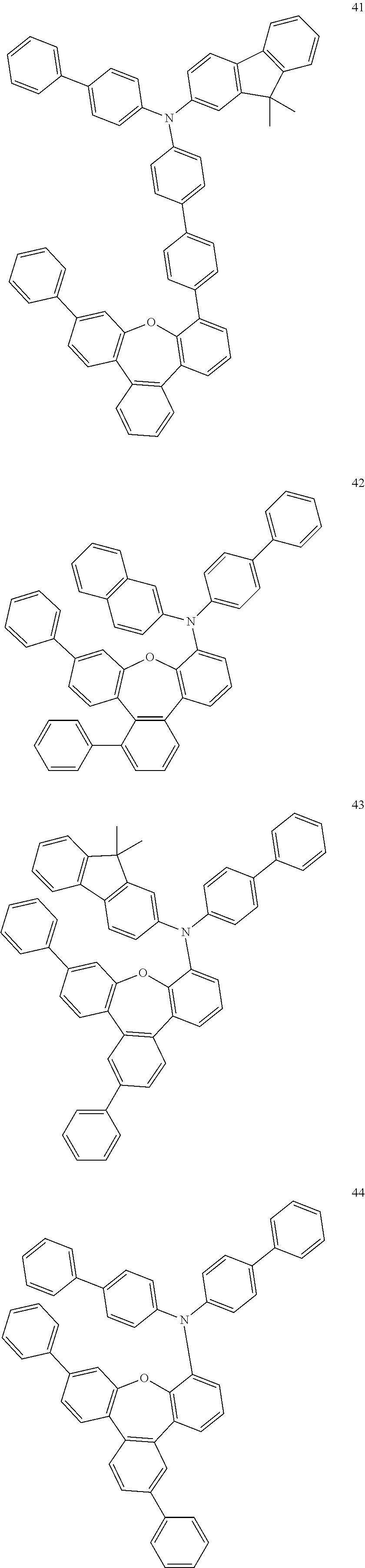 [SMILES: CC1(C)c2ccccc2-c2ccc(N(c3ccc(-c4ccccc4)cc3)c3ccc(-c4ccc(-c5cccc6c5Oc5cc(-c7ccccc7)ccc5-c5ccccc5-6)cc4)cc3)cc21 ; c1ccc(-c2ccc(N(c3ccc4ccccc4c3)c3cccc4c3Oc3cc(-c5ccccc5)ccc3-c3c(-c5ccccc5)cccc3-4)cc2)cc1 ; CC1(C)c2ccccc2-c2ccc(N(c3ccc(-c4ccccc4)cc3)c3cccc4c3Oc3cc(-c5ccccc5)ccc3-c3cc(-c5ccccc5)ccc3-4)cc21 ; c1ccc(-c2ccc(N(c3ccc(-c4ccccc4)cc3)c3cccc4c3Oc3cc(-c5ccccc5)ccc3-c3ccc(-c5ccccc5)cc3-4)cc2)cc1]

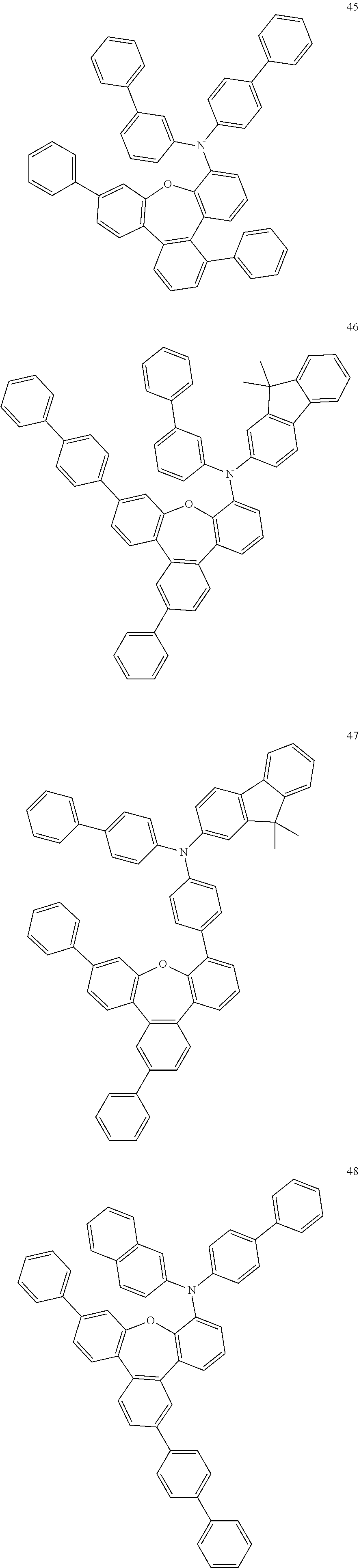 [SMILES: c1ccc(-c2ccc(N(c3cccc(-c4ccccc4)c3)c3cccc4c3Oc3cc(-c5ccccc5)ccc3-c3cccc(-c5ccccc5)c3-4)cc2)cc1 ; CC1(C)c2ccccc2-c2ccc(N(c3cccc(-c4ccccc4)c3)c3cccc4c3Oc3cc(-c5ccc(-c6ccccc6)cc5)ccc3-c3cc(-c5ccccc5)ccc3-4)cc21 ; CC1(C)c2ccccc2-c2ccc(N(c3ccc(-c4ccccc4)cc3)c3ccc(-c4cccc5c4Oc4cc(-c6ccccc6)ccc4-c4cc(-c6ccccc6)ccc4-5)cc3)cc21 ; c1ccc(-c2ccc(-c3ccc4c(c3)-c3cccc(N(c5ccc(-c6ccccc6)cc5)c5ccc6ccccc6c5)c3Oc3cc(-c5ccccc5)ccc3-4)cc2)cc1]

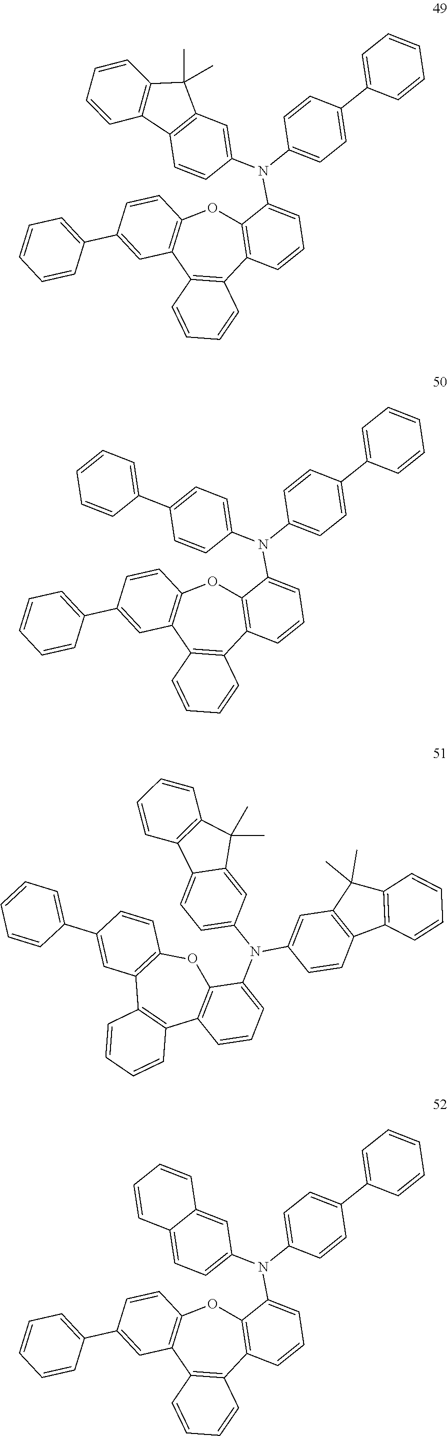 [SMILES: CC1(C)c2ccccc2-c2ccc(N(c3ccc(-c4ccccc4)cc3)c3cccc4c3Oc3ccc(-c5ccccc5)cc3-c3ccccc3-4)cc21 ; c1ccc(-c2ccc(N(c3ccc(-c4ccccc4)cc3)c3cccc4c3Oc3ccc(-c5ccccc5)cc3-c3ccccc3-4)cc2)cc1 ; CC1(C)c2ccccc2-c2ccc(N(c3ccc4c(c3)C(C)(C)c3ccccc3-4)c3cccc4c3Oc3ccc(-c5ccccc5)cc3-c3ccccc3-4)cc21 ; c1ccc(-c2ccc(N(c3ccc4ccccc4c3)c3cccc4c3Oc3ccc(-c5ccccc5)cc3-c3ccccc3-4)cc2)cc1]

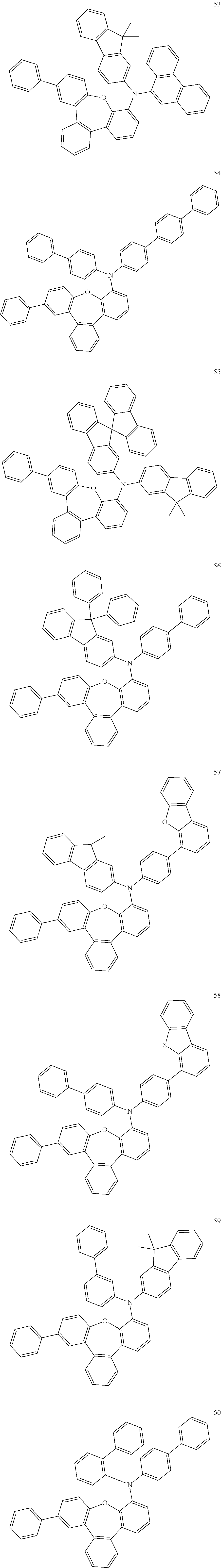

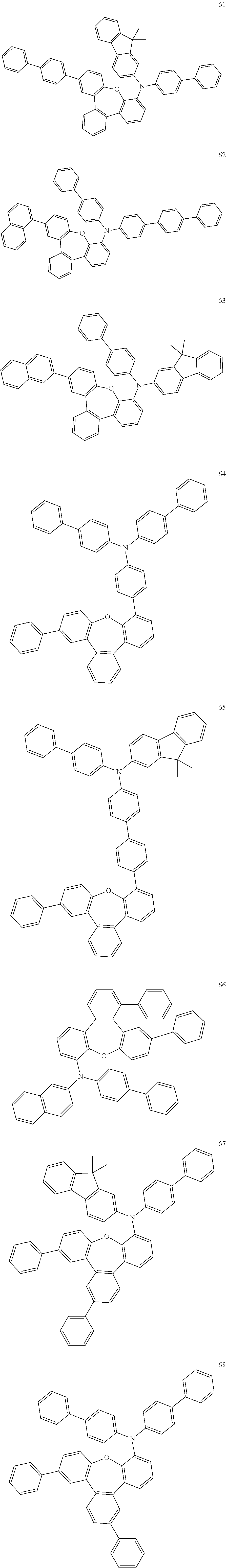

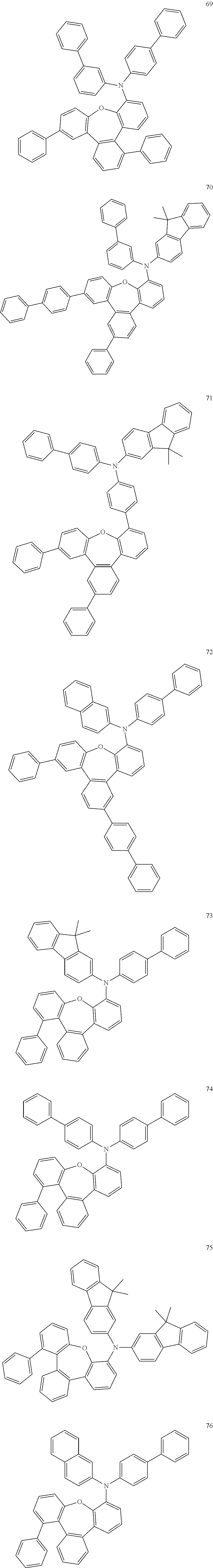

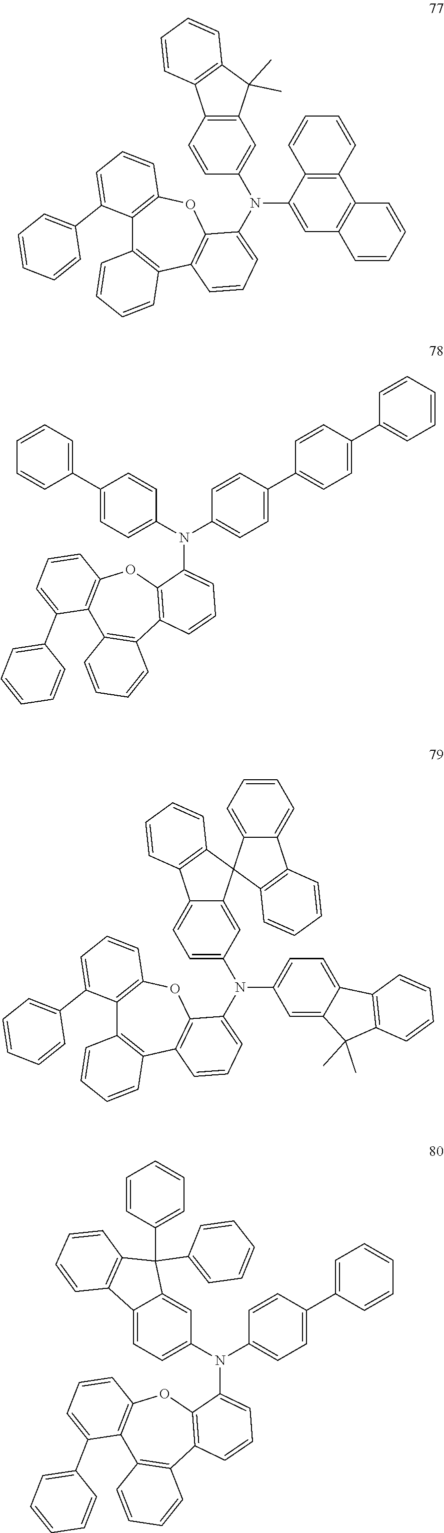 [SMILES: CC1(C)c2ccccc2-c2ccc(N(c3cccc4c3Oc3cccc(-c5ccccc5)c3-c3ccccc3-4)c3cc4ccccc4c4ccccc34)cc21 ; c1ccc(-c2ccc(-c3ccc(N(c4ccc(-c5ccccc5)cc4)c4cccc5c4Oc4cccc(-c6ccccc6)c4-c4ccccc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cccc(-c5ccccc5)c3-c3ccccc3-4)cc21 ; c1ccc(-c2ccc(N(c3ccc4c(c3)C(c3ccccc3)(c3ccccc3)c3ccccc3-4)c3cccc4c3Oc3cccc(-c5ccccc5)c3-c3ccccc3-4)cc2)cc1]

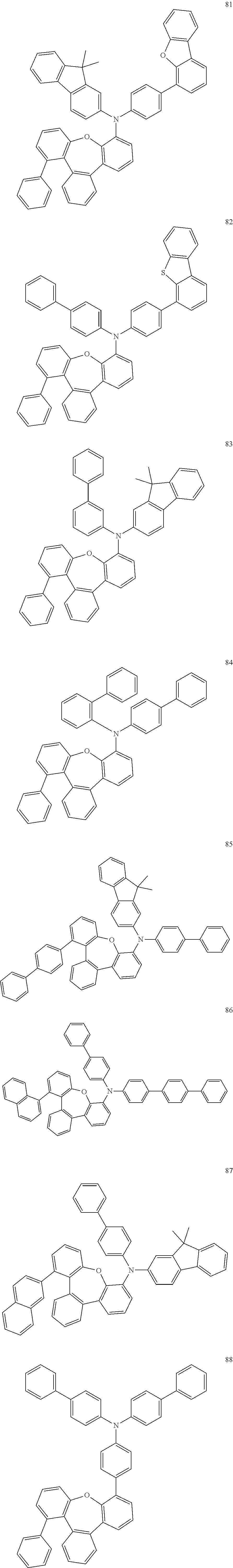

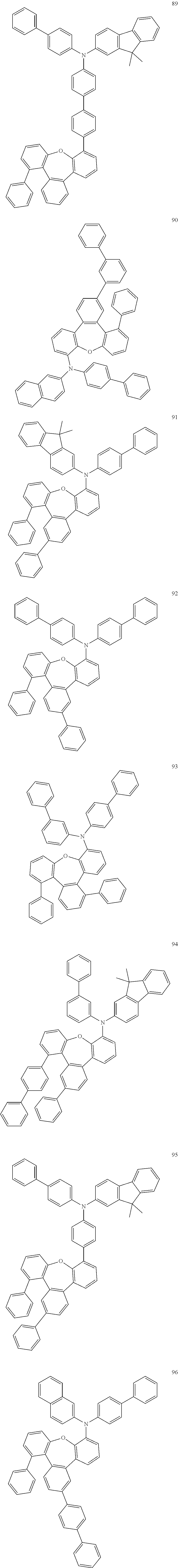

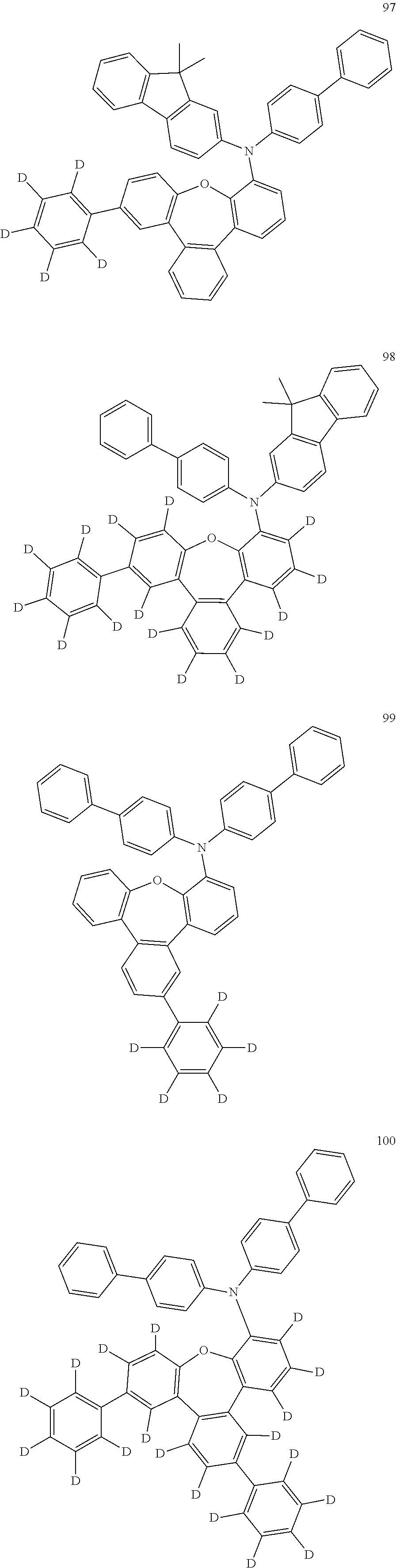

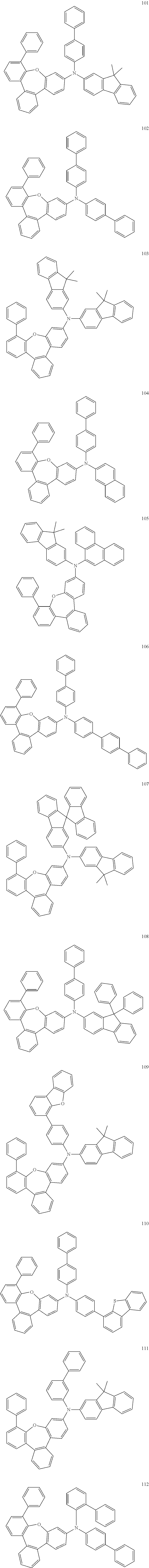

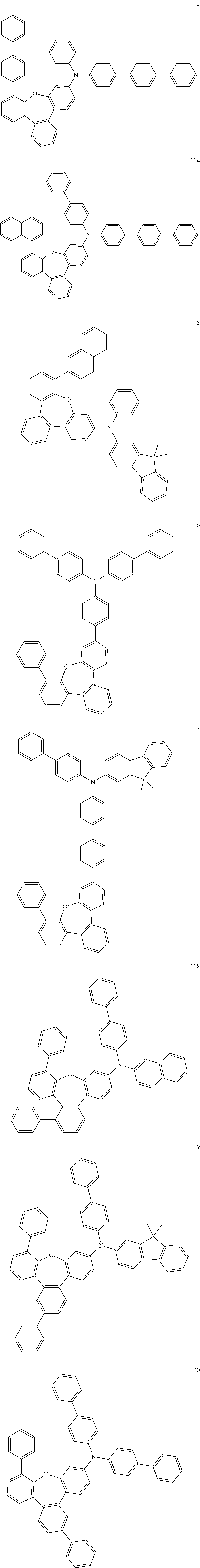

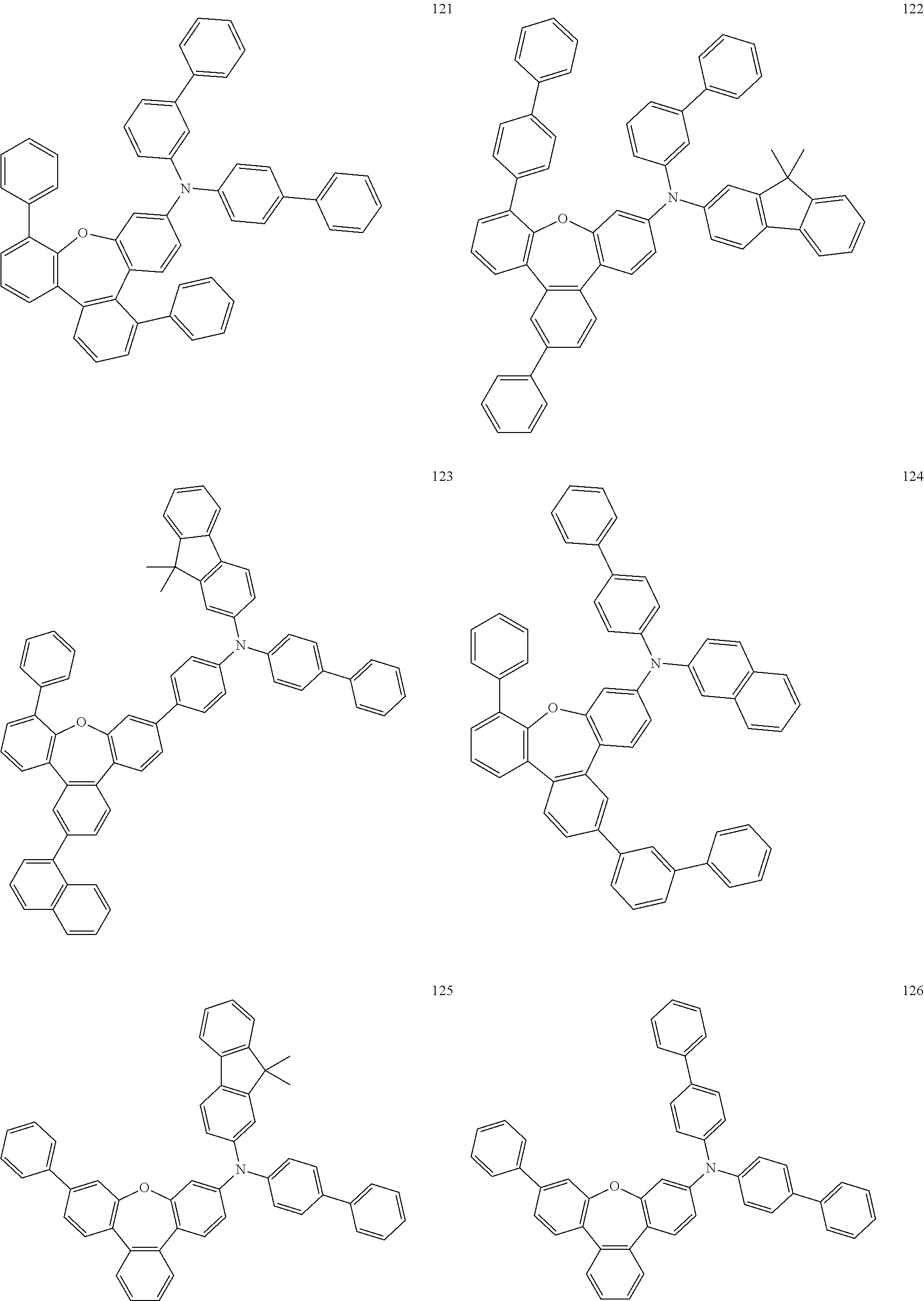

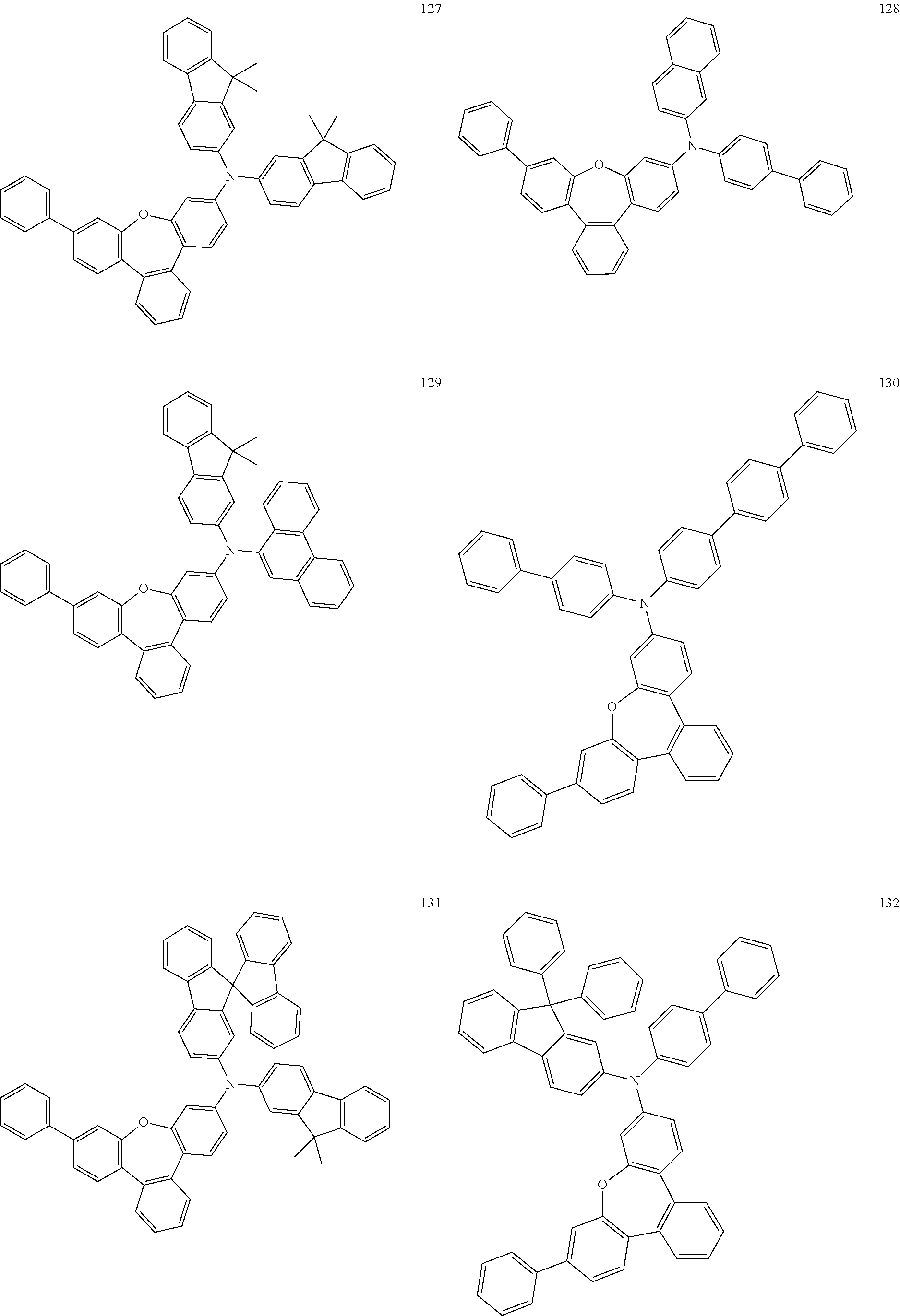

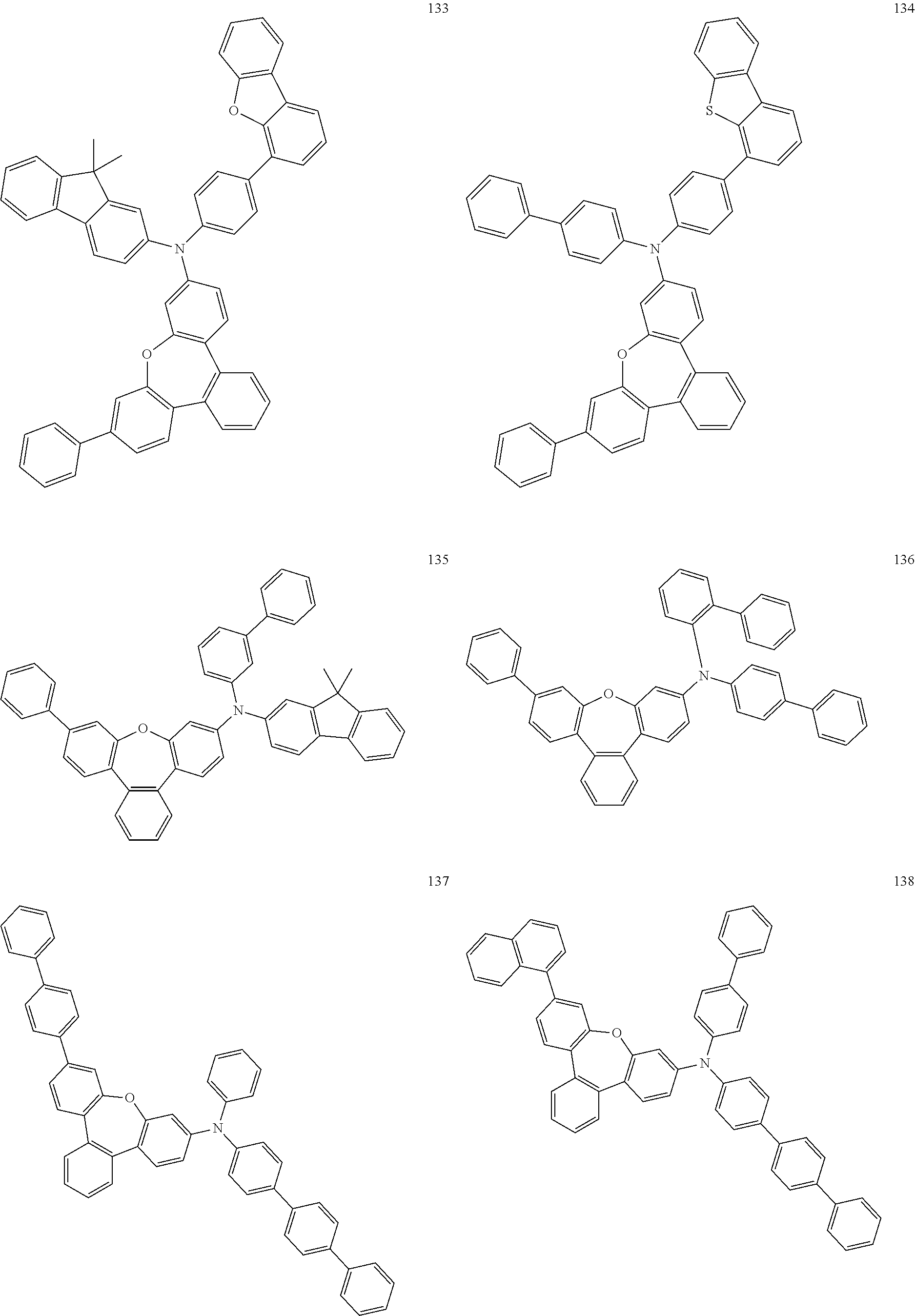

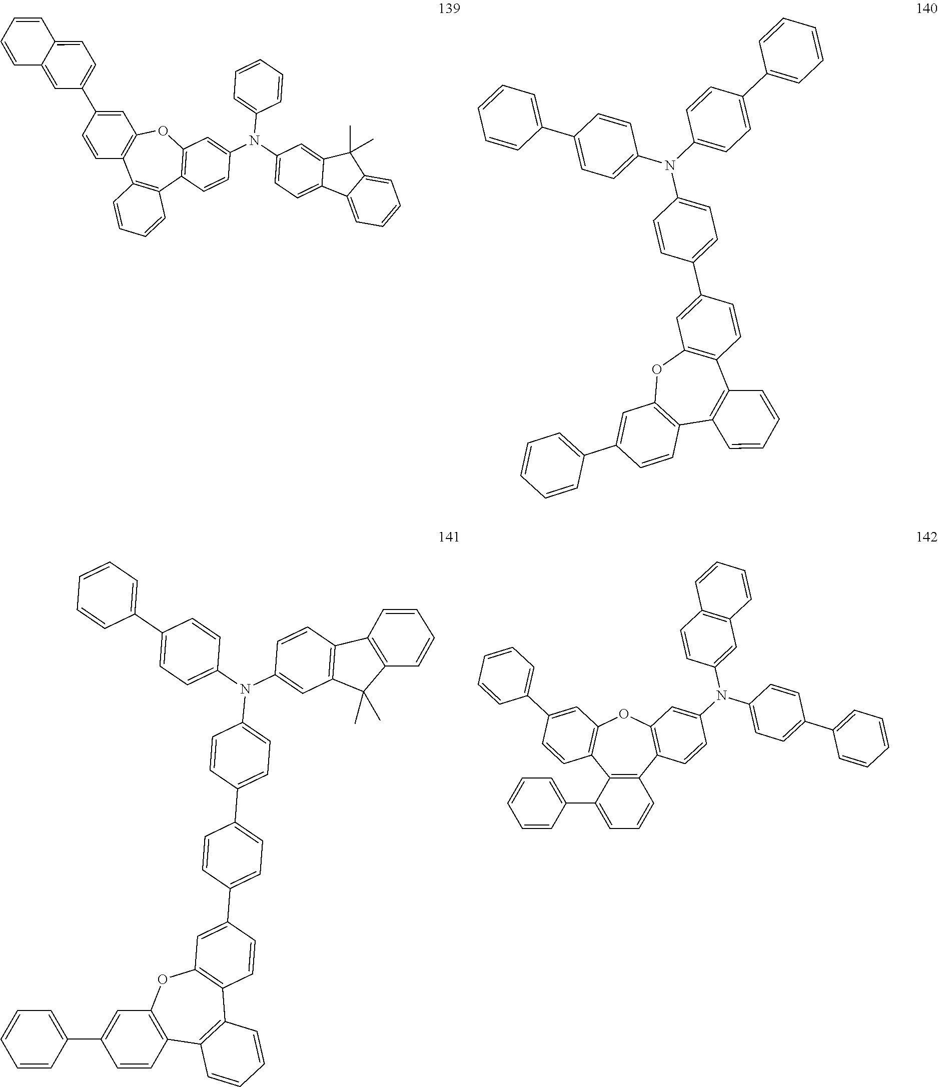 [SMILES: CC1(C)c2ccccc2-c2ccc(N(c3ccccc3)c3ccc4c(c3)Oc3cc(-c5ccc6ccccc6c5)ccc3-c3ccccc3-4)cc21 ; c1ccc(-c2ccc(N(c3ccc(-c4ccccc4)cc3)c3ccc(-c4ccc5c(c4)Oc4cc(-c6ccccc6)ccc4-c4ccccc4-5)cc3)cc2)cc1 ; CC1(C)c2ccccc2-c2ccc(N(c3ccc(-c4ccccc4)cc3)c3ccc(-c4ccc(-c5ccc6c(c5)Oc5cc(-c7ccccc7)ccc5-c5ccccc5-6)cc4)cc3)cc21 ; c1ccc(-c2ccc(N(c3ccc4c(c3)Oc3cc(-c5ccccc5)ccc3-c3c(-c5ccccc5)cccc3-4)c3ccc4ccccc4c3)cc2)cc1]

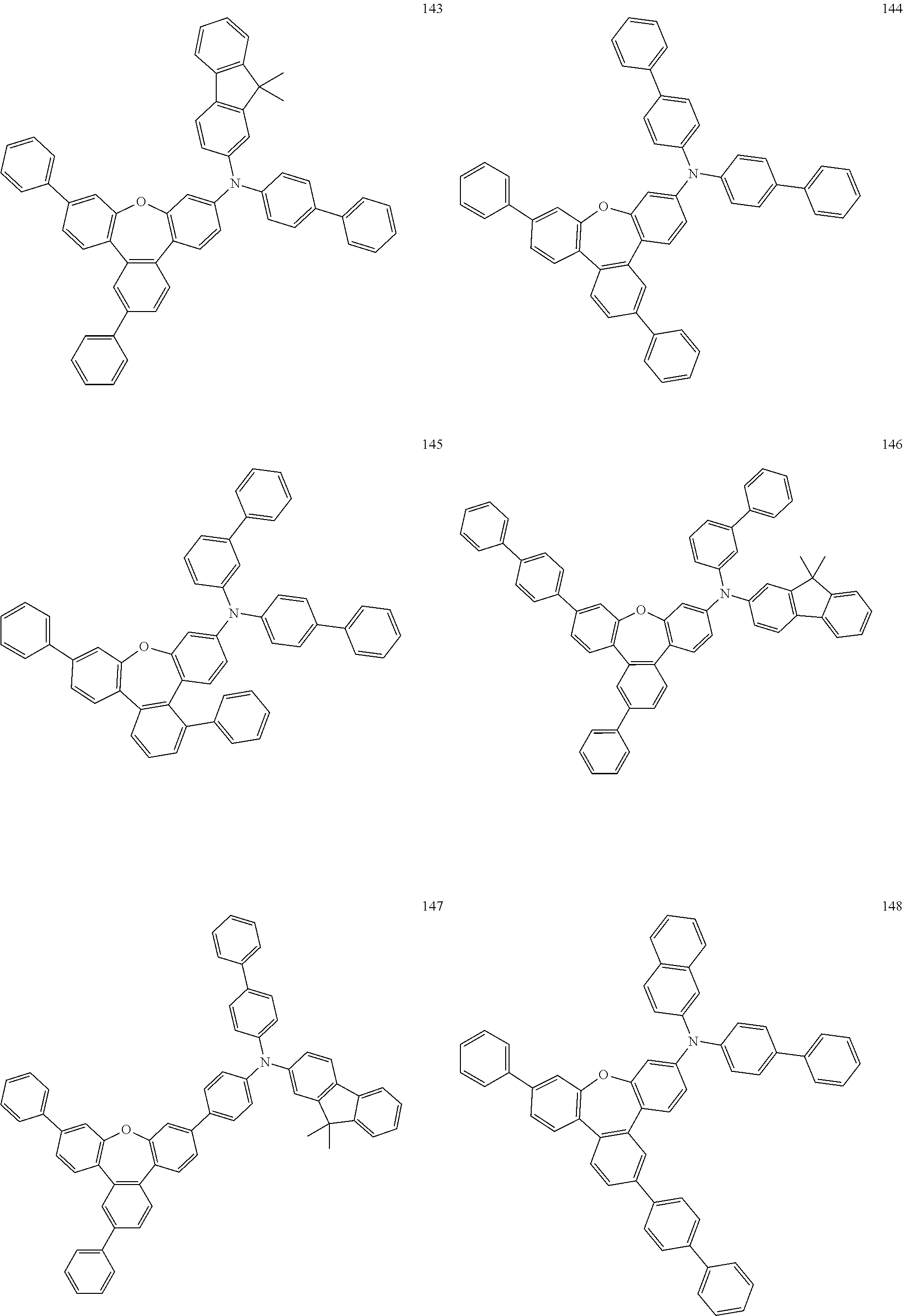

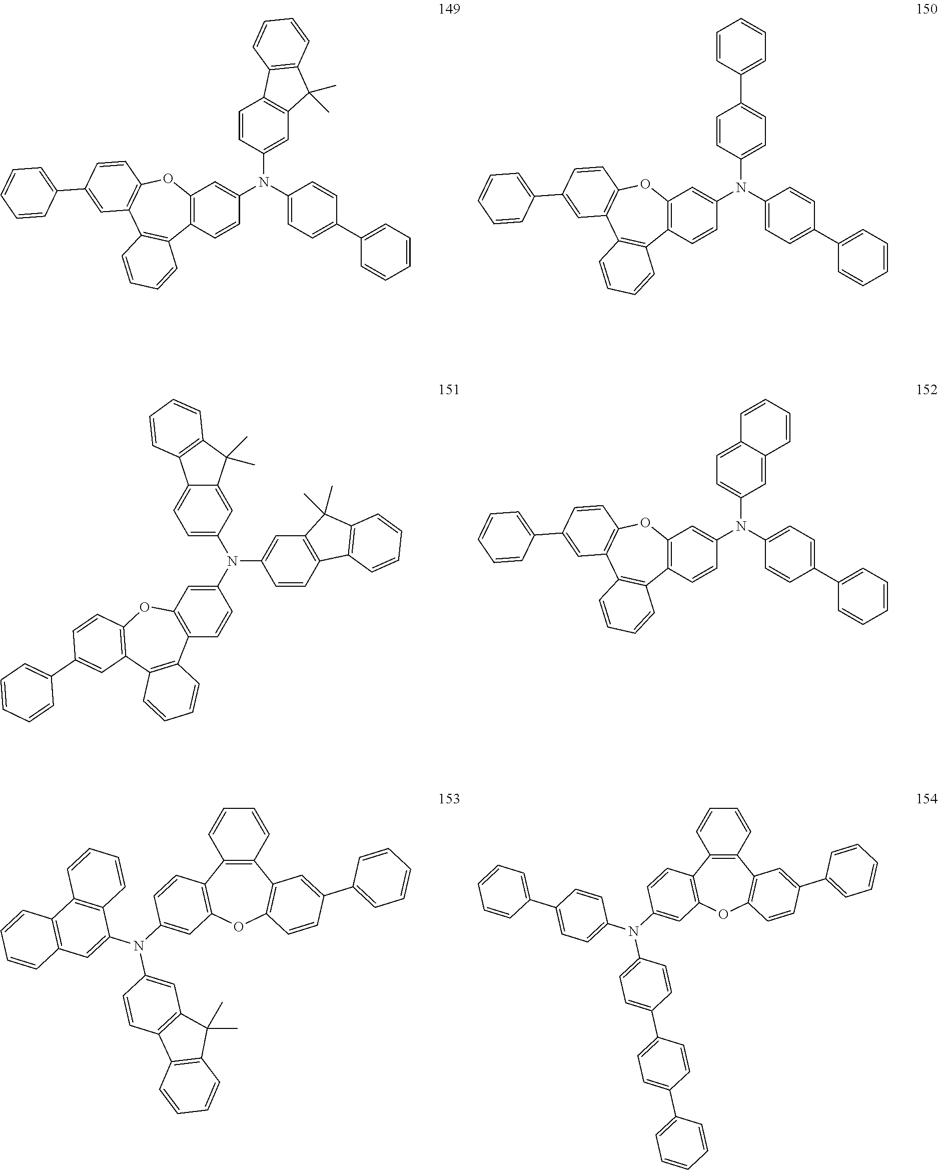

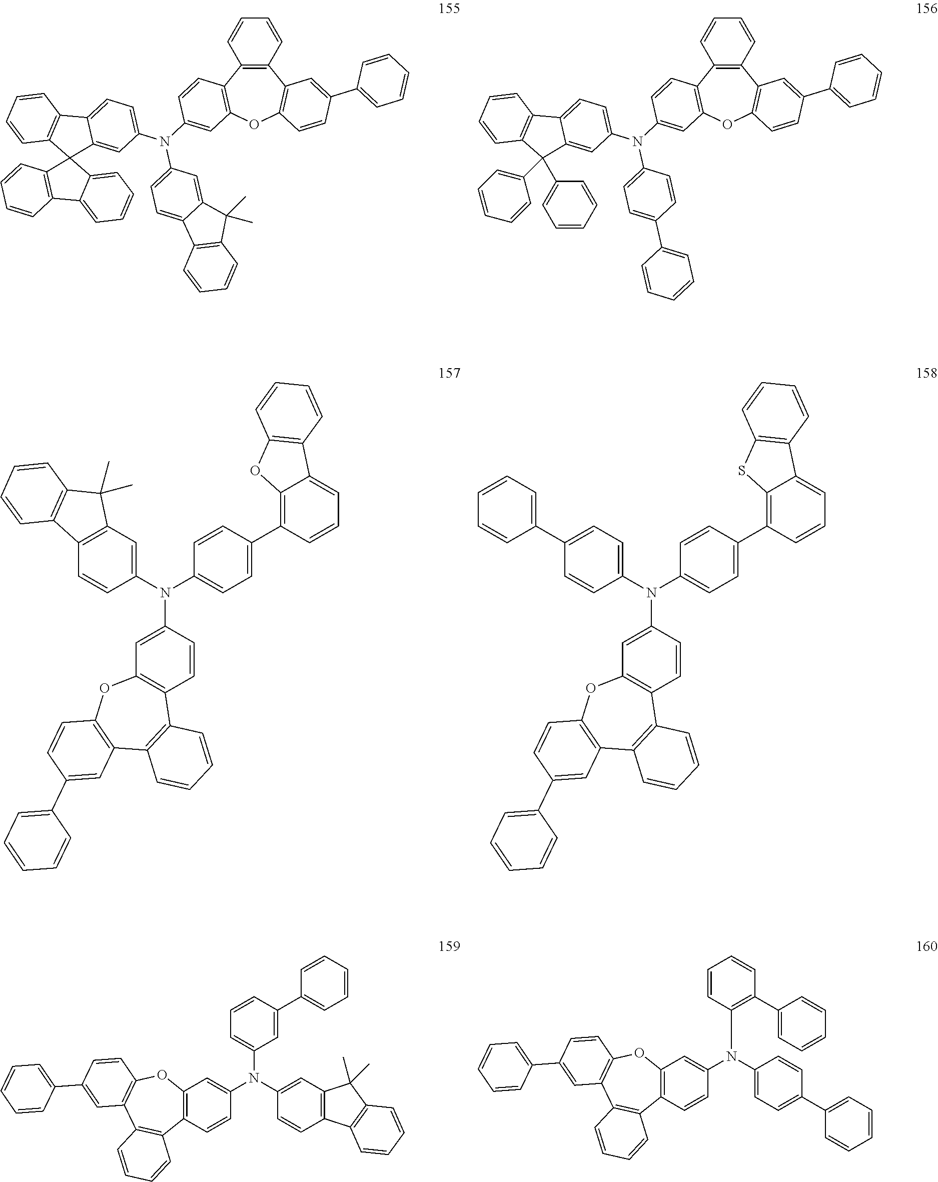

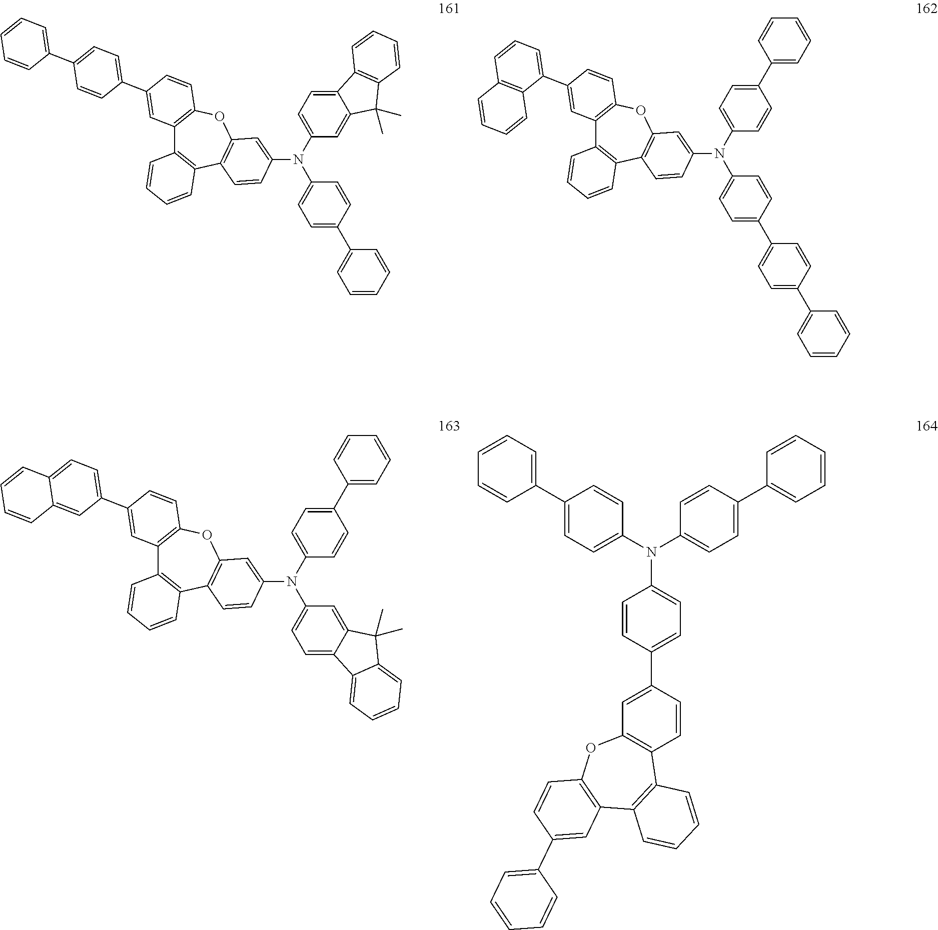 [SMILES: CC1(C)c2ccccc2-c2ccc(N(c3ccc(-c4ccccc4)cc3)c3ccc4c(c3)Oc3ccc(-c5ccc(-c6ccccc6)cc5)cc3-c3ccccc3-4)cc21 ; c1ccc(-c2ccc(-c3ccc(N(c4ccc(-c5ccccc5)cc4)c4ccc5c(c4)Oc4ccc(-c6cccc7ccccc67)cc4-c4ccccc4-5)cc3)cc2)cc1 ; CC1(C)c2ccccc2-c2ccc(N(c3ccc(-c4ccccc4)cc3)c3ccc4c(c3)Oc3ccc(-c5ccc6ccccc6c5)cc3-c3ccccc3-4)cc21 ; c1ccc(-c2ccc(N(c3ccc(-c4ccccc4)cc3)c3ccc(-c4ccc5c(c4)Oc4ccc(-c6ccccc6)cc4-c4ccccc4-5)cc3)cc2)cc1]

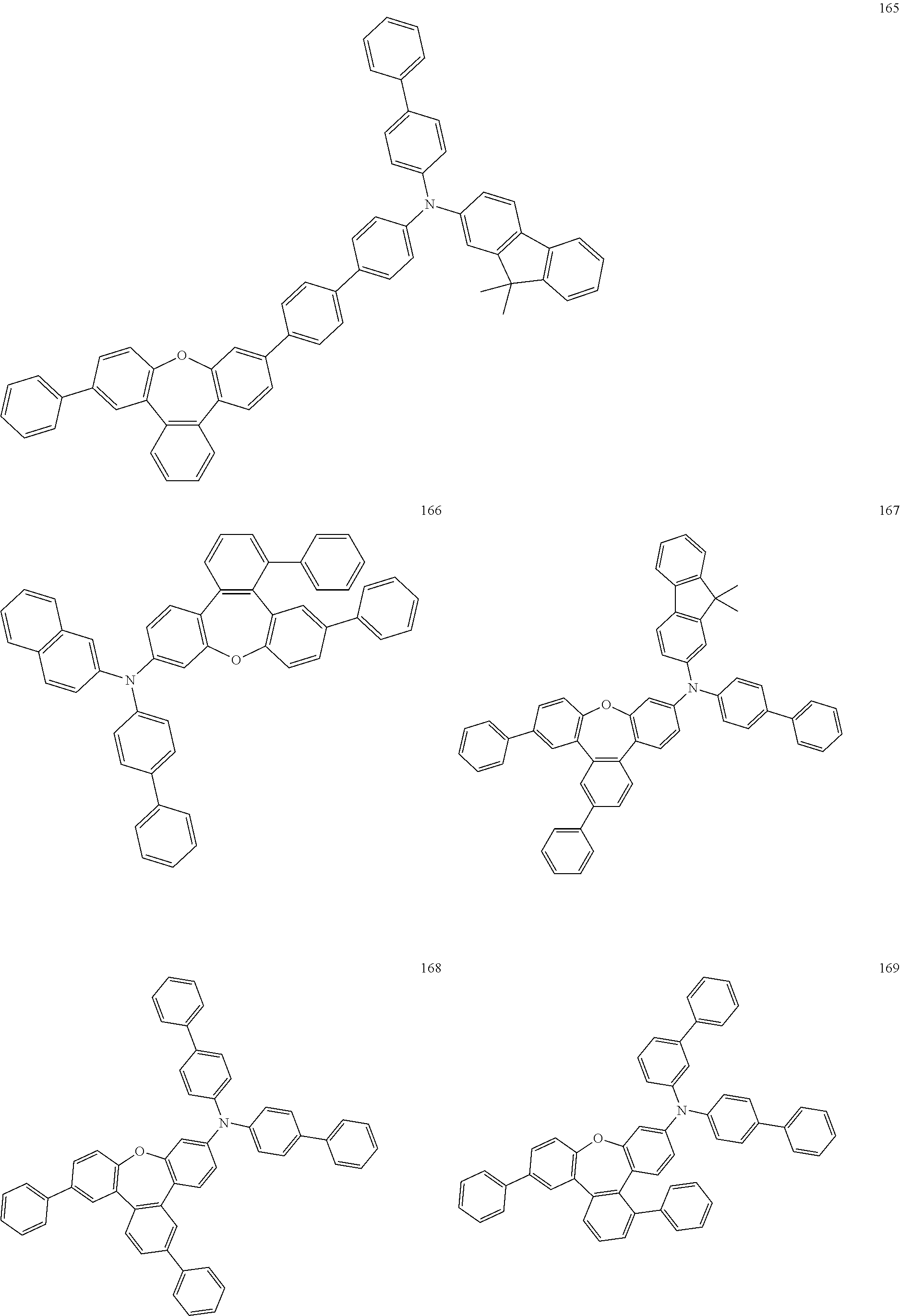

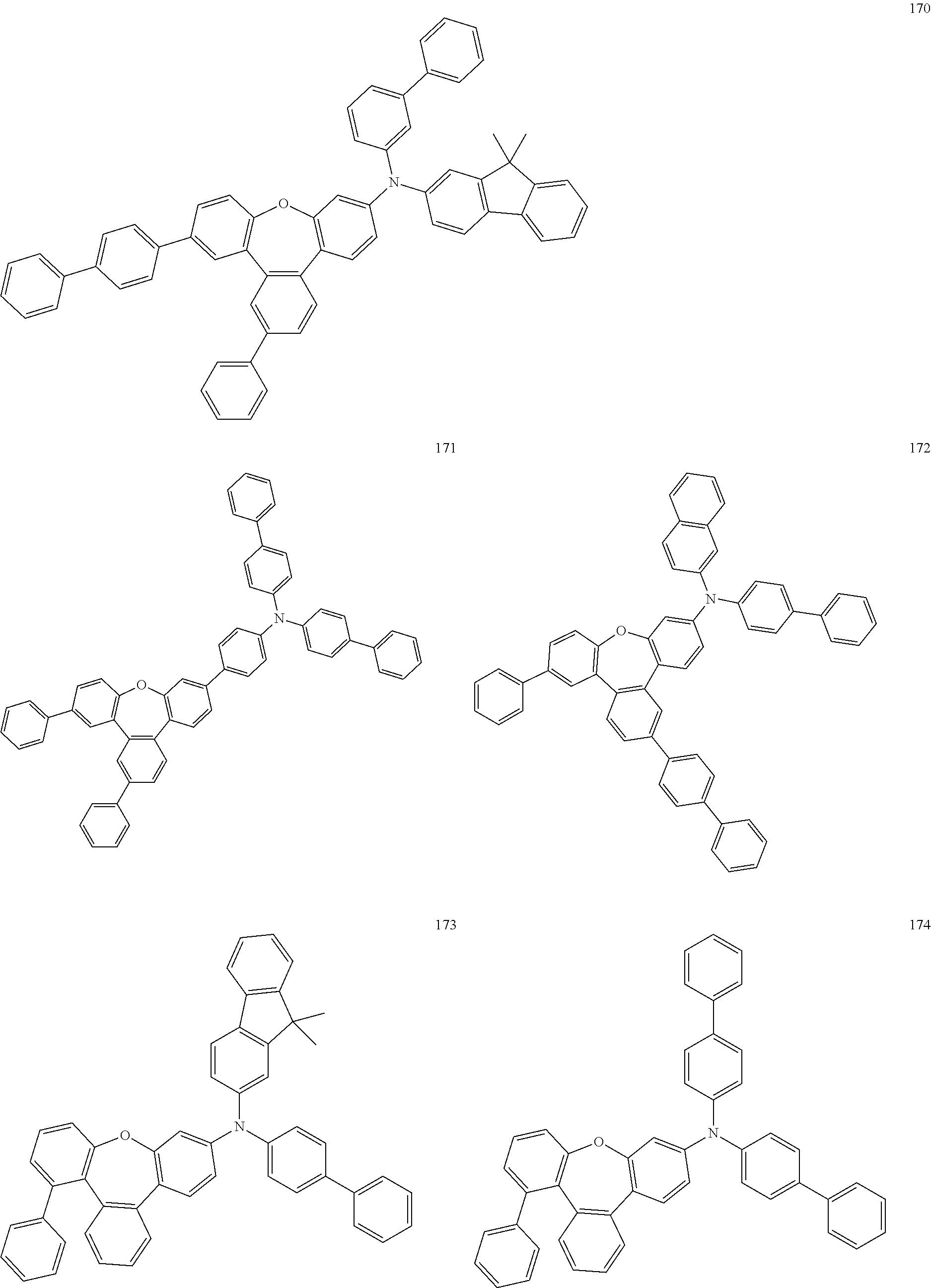

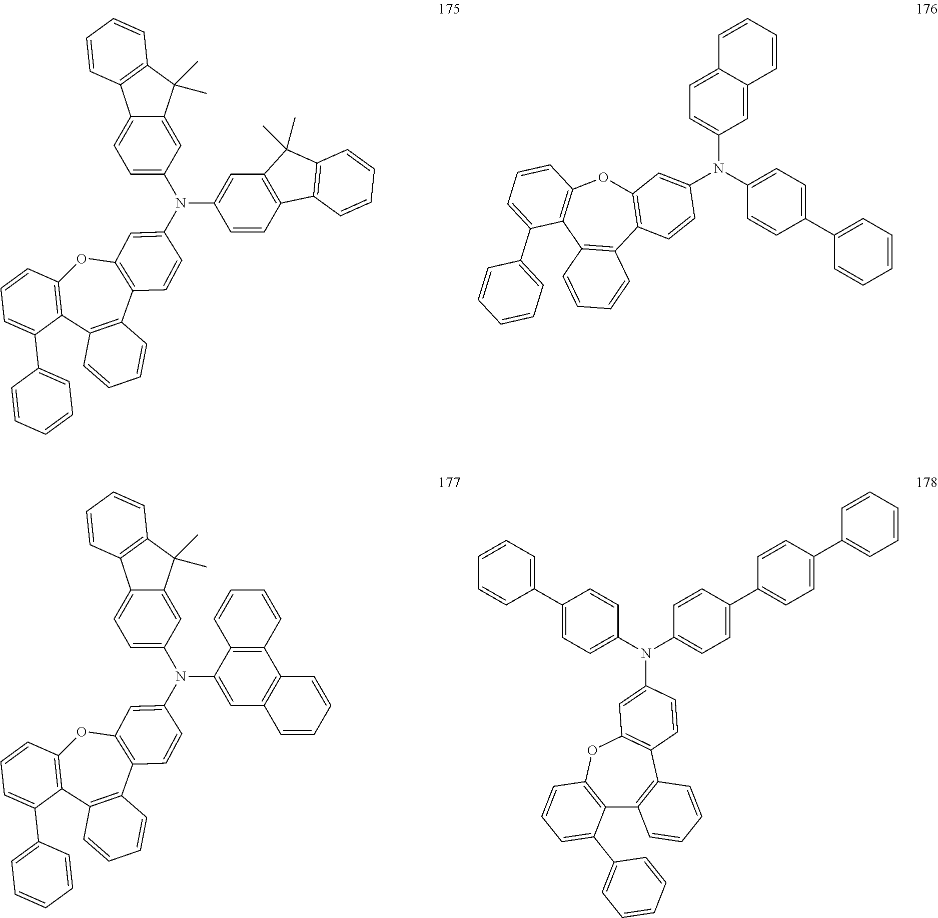 [SMILES: CC1(C)c2ccccc2-c2ccc(N(c3ccc4c(c3)Oc3cccc(-c5ccccc5)c3-c3ccccc3-4)c3ccc4c(c3)C(C)(C)c3ccccc3-4)cc21 ; c1ccc(-c2ccc(N(c3ccc4c(c3)Oc3cccc(-c5ccccc5)c3-c3ccccc3-4)c3ccc4ccccc4c3)cc2)cc1 ; CC1(C)c2ccccc2-c2ccc(N(c3ccc4c(c3)Oc3cccc(-c5ccccc5)c3-c3ccccc3-4)c3cc4ccccc4c4ccccc34)cc21 ; c1ccc(-c2ccc(-c3ccc(N(c4ccc(-c5ccccc5)cc4)c4ccc5c(c4)Oc4cccc(-c6ccccc6)c4-c4ccccc4-5)cc3)cc2)cc1]

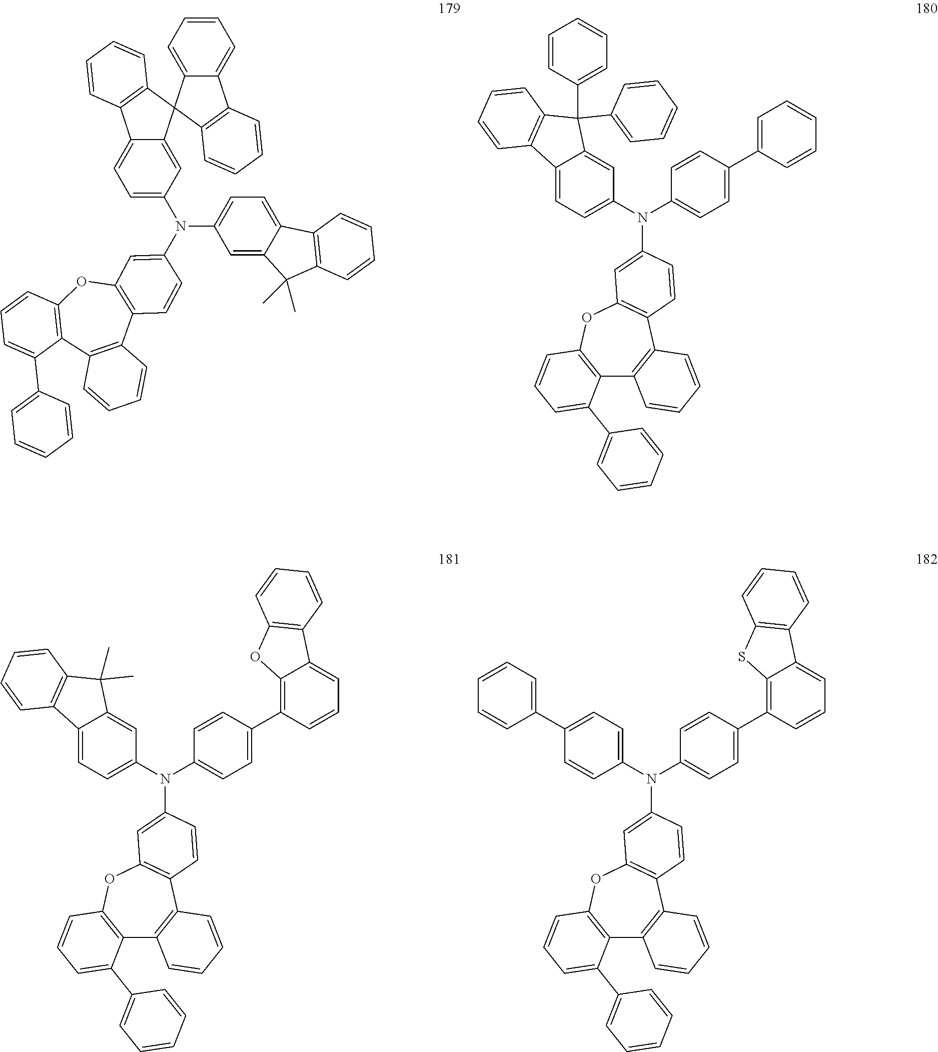 [SMILES: CC1(C)c2ccccc2-c2ccc(N(c3ccc4c(c3)Oc3cccc(-c5ccccc5)c3-c3ccccc3-4)c3ccc4c(c3)C3(c5ccccc5-c5ccccc53)c3ccccc3-4)cc21 ; c1ccc(-c2ccc(N(c3ccc4c(c3)Oc3cccc(-c5ccccc5)c3-c3ccccc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cccc(-c5ccccc5)c3-c3ccccc3-4)cc21 ; c1ccc(-c2ccc(N(c3ccc(-c4cccc5c4sc4ccccc45)cc3)c3ccc4c(c3)Oc3cccc(-c5ccccc5)c3-c3ccccc3-4)cc2)cc1]

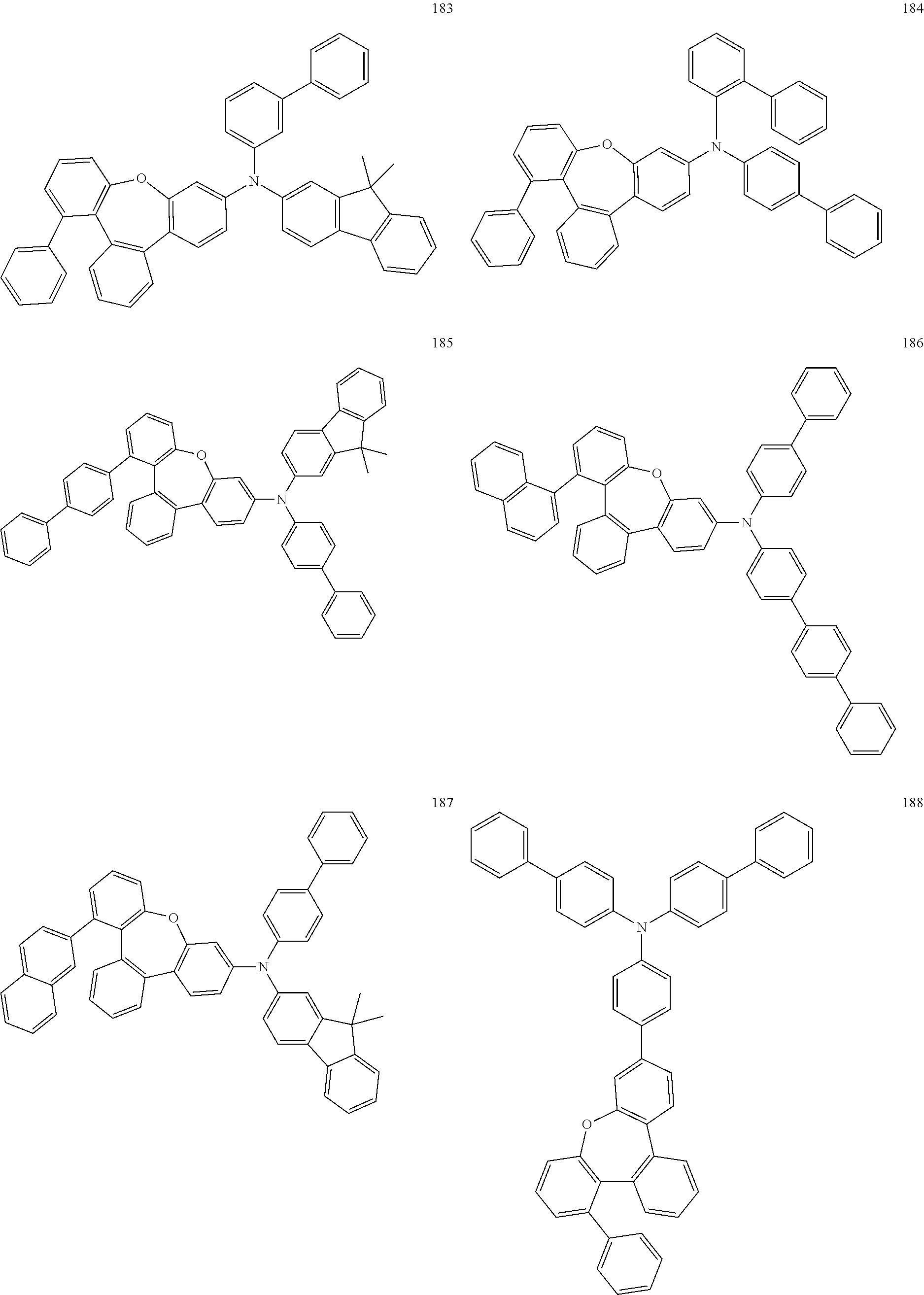

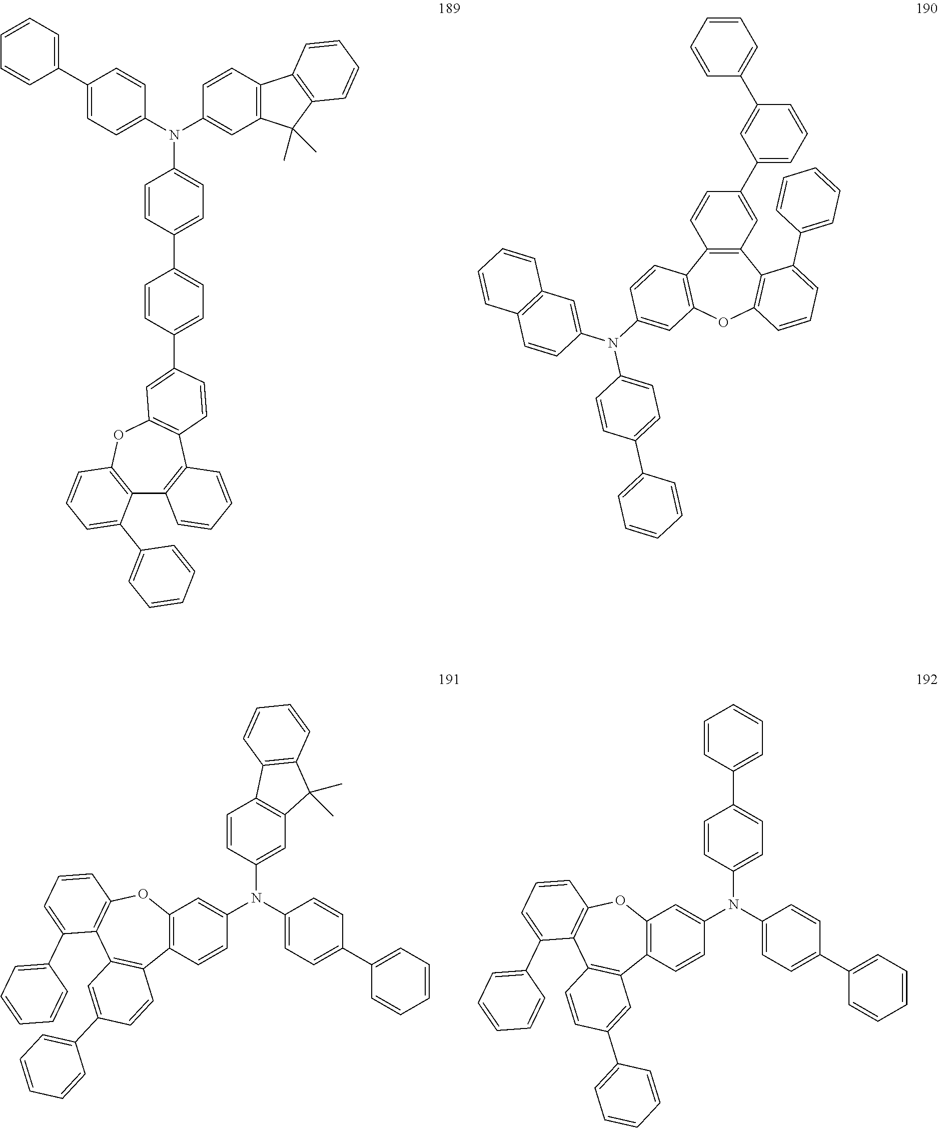 [SMILES: CC1(C)c2ccccc2-c2ccc(N(c3ccc(-c4ccccc4)cc3)c3ccc(-c4ccc(-c5ccc6c(c5)Oc5cccc(-c7ccccc7)c5-c5ccccc5-6)cc4)cc3)cc21 ; c1ccc(-c2ccc(N(c3ccc4c(c3)Oc3cccc(-c5ccccc5)c3-c3cc(-c5cccc(-c6ccccc6)c5)ccc3-4)c3ccc4ccccc4c3)cc2)cc1 ; CC1(C)c2ccccc2-c2ccc(N(c3ccc(-c4ccccc4)cc3)c3ccc4c(c3)Oc3cccc(-c5ccccc5)c3-c3cc(-c5ccccc5)ccc3-4)cc21 ; c1ccc(-c2ccc(N(c3ccc(-c4ccccc4)cc3)c3ccc4c(c3)Oc3cccc(-c5ccccc5)c3-c3ccc(-c5ccccc5)cc3-4)cc2)cc1]

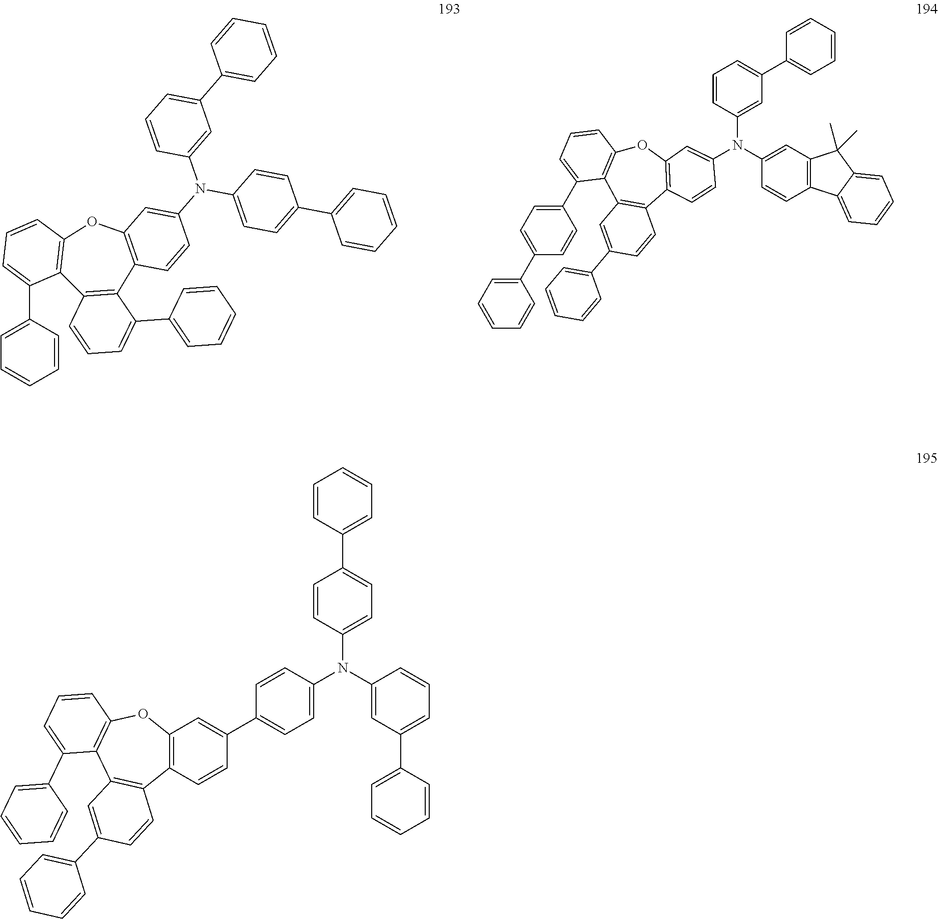 [SMILES: c1ccc(-c2ccc(N(c3cccc(-c4ccccc4)c3)c3ccc4c(c3)Oc3cccc(-c5ccccc5)c3-c3cccc(-c5ccccc5)c3-4)cc2)cc1 ; CC1(C)c2ccccc2-c2ccc(N(c3cccc(-c4ccccc4)c3)c3ccc4c(c3)Oc3cccc(-c5ccc(-c6ccccc6)cc5)c3-c3cc(-c5ccccc5)ccc3-4)cc21 ; c1ccc(-c2ccc(N(c3ccc(-c4ccc5c(c4)Oc4cccc(-c6ccccc6)c4-c4cc(-c6ccccc6)ccc4-5)cc3)c3cccc(-c4ccccc4)c3)cc2)cc1]

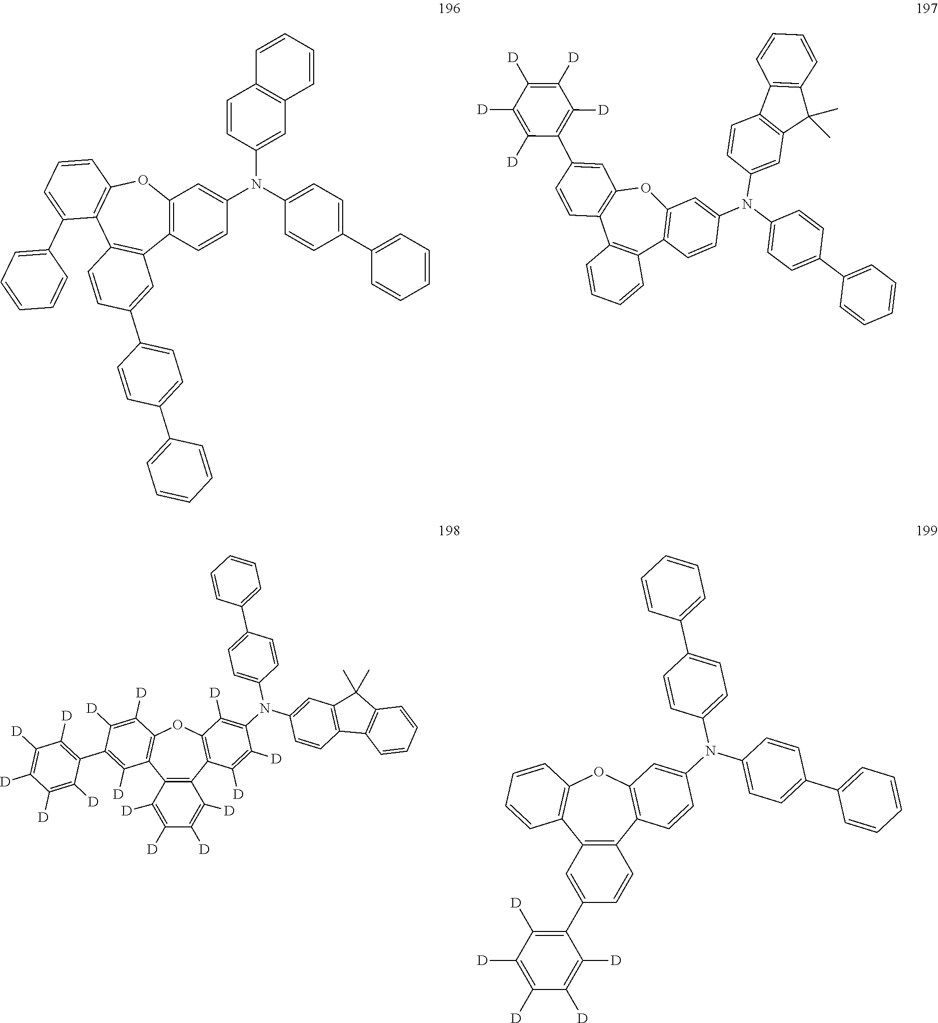

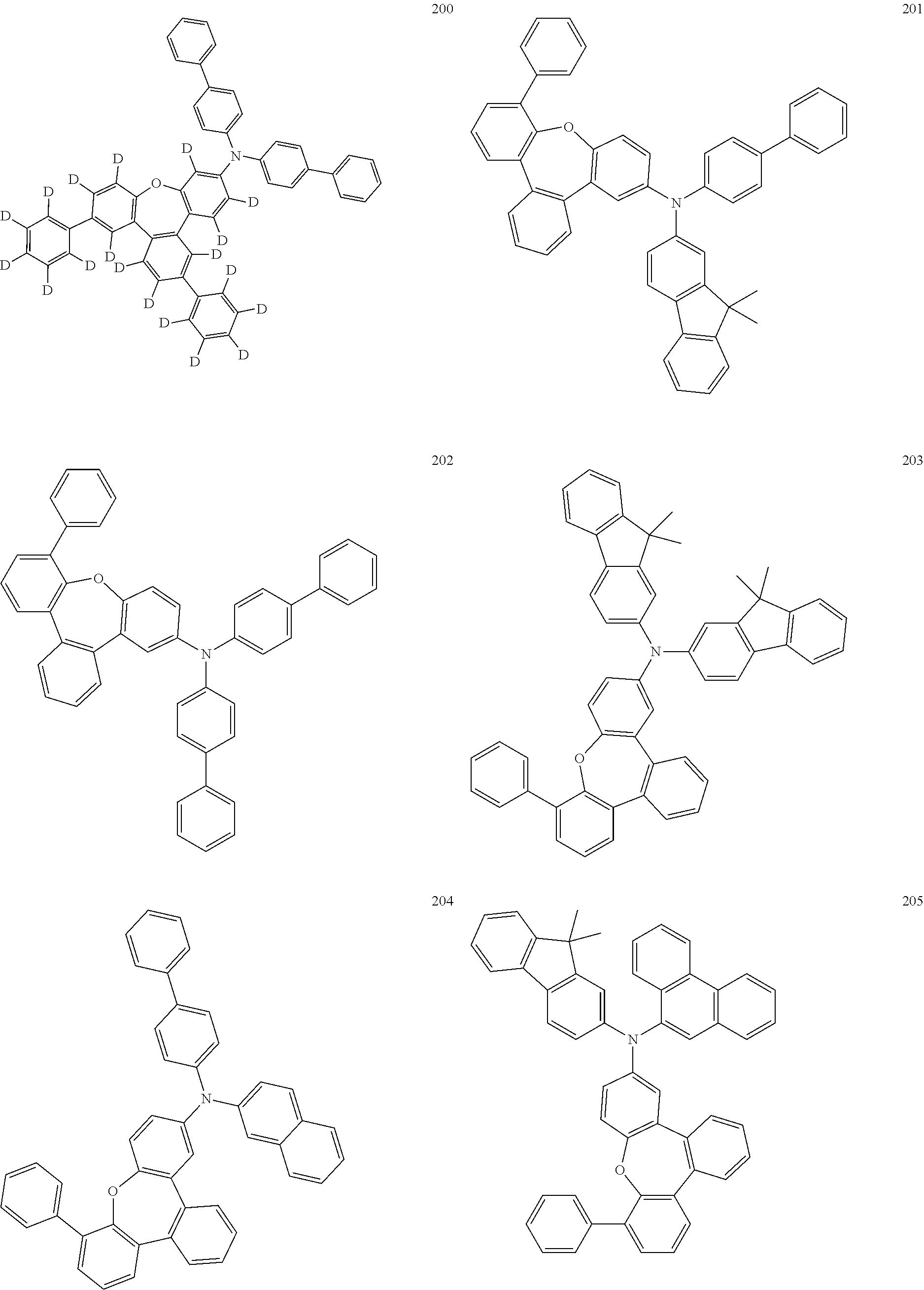

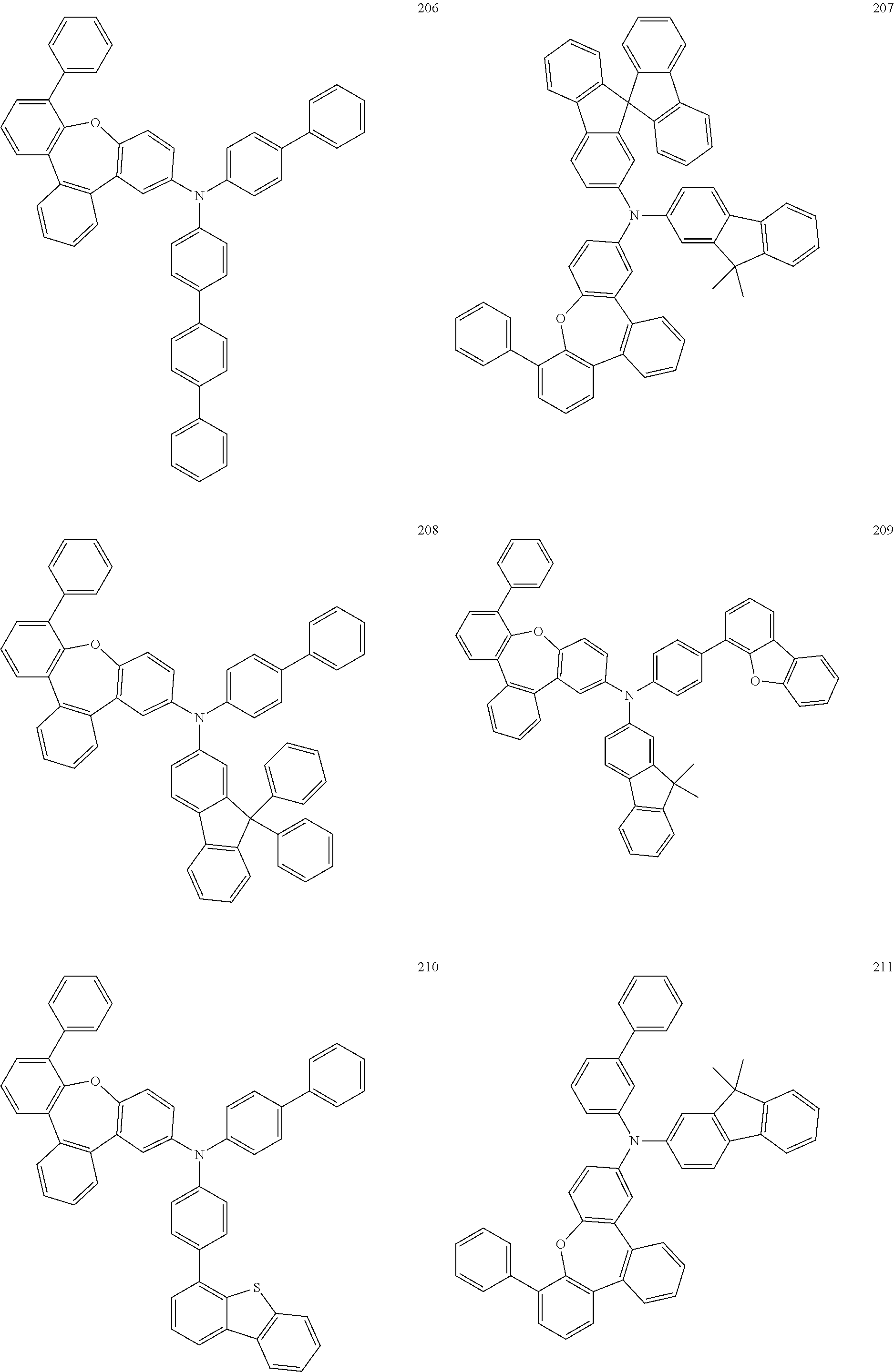

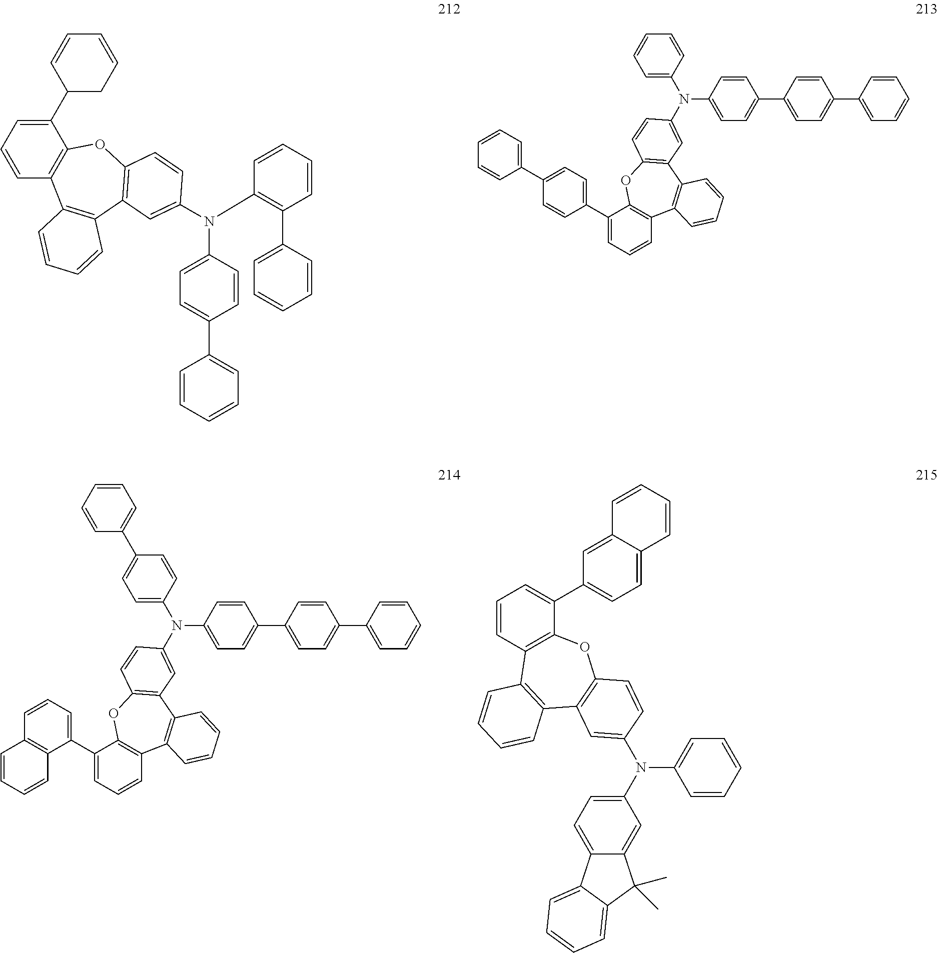 [SMILES: C1=CCC(c2cccc3c2Oc2ccc(N(c4ccc(-c5ccccc5)cc4)c4ccccc4-c4ccccc4)cc2-c2ccccc2-3)C=C1 ; c1ccc(-c2ccc(-c3ccc(N(c4ccccc4)c4ccc5c(c4)-c4ccccc4-c4cccc(-c6ccc(-c7ccccc7)cc6)c4O5)cc3)cc2)cc1 ; c1ccc(-c2ccc(-c3ccc(N(c4ccc(-c5ccccc5)cc4)c4ccc5c(c4)-c4ccccc4-c4cccc(-c6cccc7ccccc67)c4O5)cc3)cc2)cc1 ; CC1(C)c2ccccc2-c2ccc(N(c3ccccc3)c3ccc4c(c3)-c3ccccc3-c3cccc(-c5ccc6ccccc6c5)c3O4)cc21]

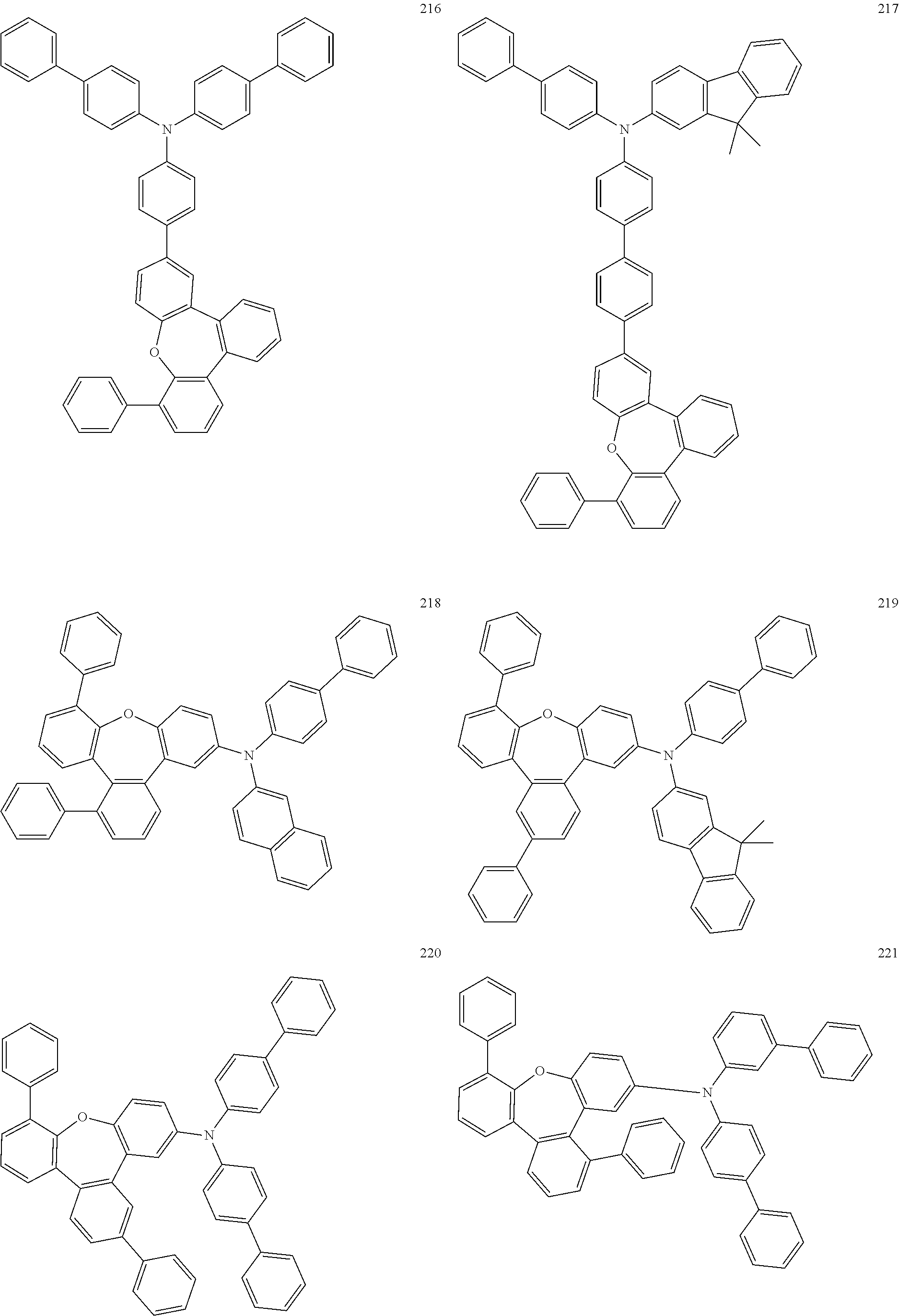

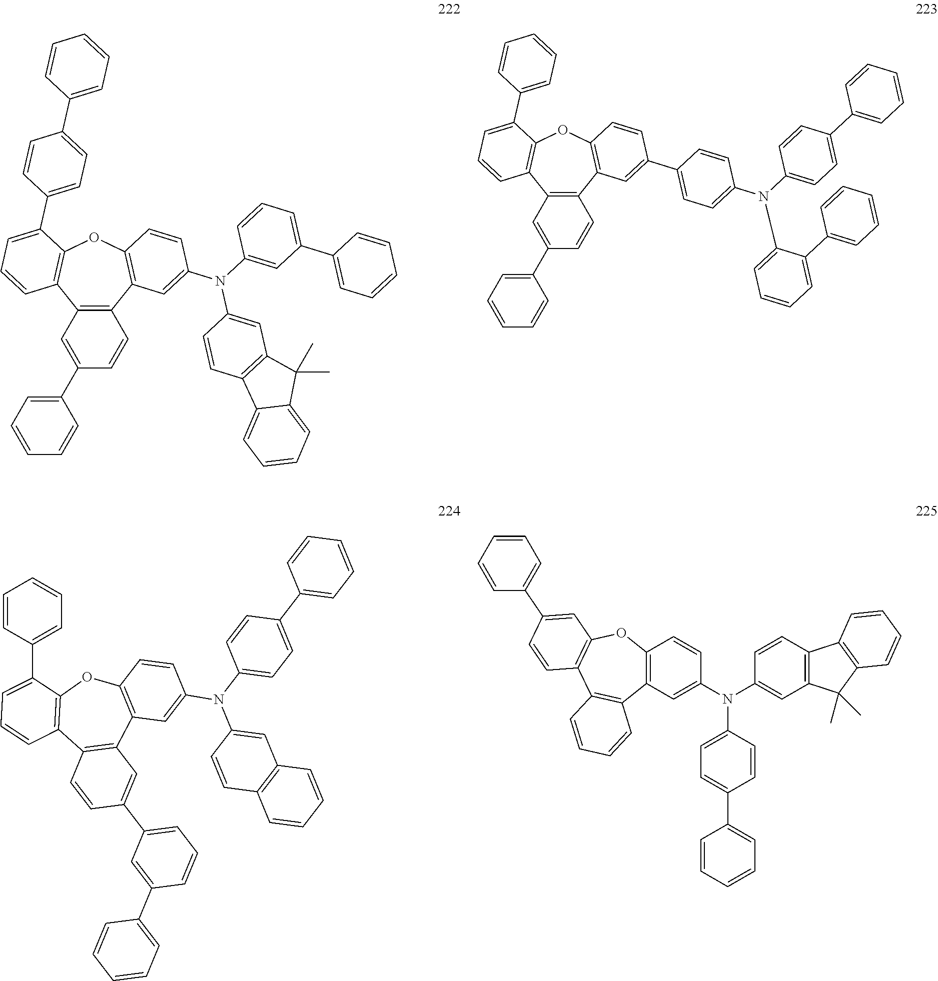 [SMILES: CC1(C)c2ccccc2-c2ccc(N(c3cccc(-c4ccccc4)c3)c3ccc4c(c3)-c3ccc(-c5ccccc5)cc3-c3cccc(-c5ccc(-c6ccccc6)cc5)c3O4)cc21 ; c1ccc(-c2ccc(N(c3ccc(-c4ccc5c(c4)-c4ccc(-c6ccccc6)cc4-c4cccc(-c6ccccc6)c4O5)cc3)c3ccccc3-c3ccccc3)cc2)cc1 ; c1ccc(-c2ccc(N(c3ccc4c(c3)-c3cc(-c5cccc(-c6ccccc6)c5)ccc3-c3cccc(-c5ccccc5)c3O4)c3ccc4ccccc4c3)cc2)cc1 ; CC1(C)c2ccccc2-c2ccc(N(c3ccc(-c4ccccc4)cc3)c3ccc4c(c3)-c3ccccc3-c3ccc(-c5ccccc5)cc3O4)cc21]

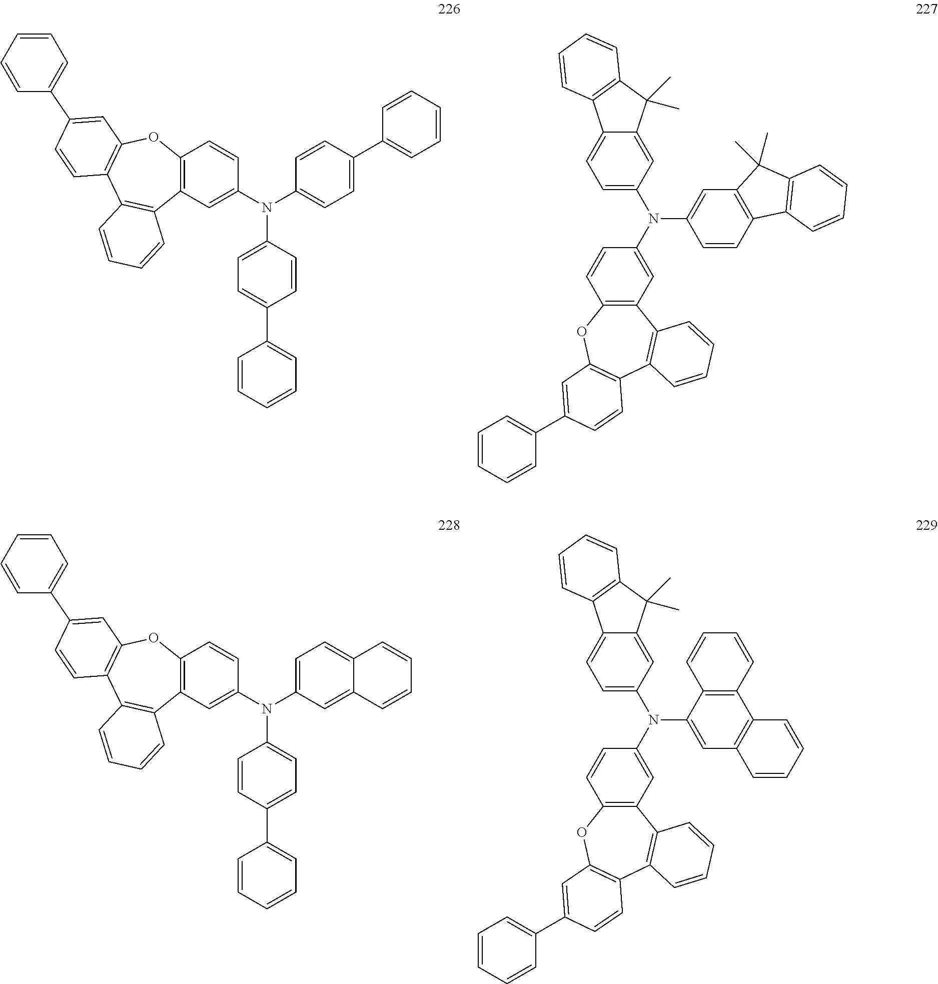 [SMILES: c1ccc(-c2ccc(N(c3ccc(-c4ccccc4)cc3)c3ccc4c(c3)-c3ccccc3-c3ccc(-c5ccccc5)cc3O4)cc2)cc1 ; CC1(C)c2ccccc2-c2ccc(N(c3ccc4c(c3)-c3ccccc3-c3ccc(-c5ccccc5)cc3O4)c3ccc4c(c3)C(C)(C)c3ccccc3-4)cc21 ; c1ccc(-c2ccc(N(c3ccc4c(c3)-c3ccccc3-c3ccc(-c5ccccc5)cc3O4)c3ccc4ccccc4c3)cc2)cc1 ; CC1(C)c2ccccc2-c2ccc(N(c3ccc4c(c3)-c3ccccc3-c3ccc(-c5ccccc5)cc3O4)c3cc4ccccc4c4ccccc34)cc21]

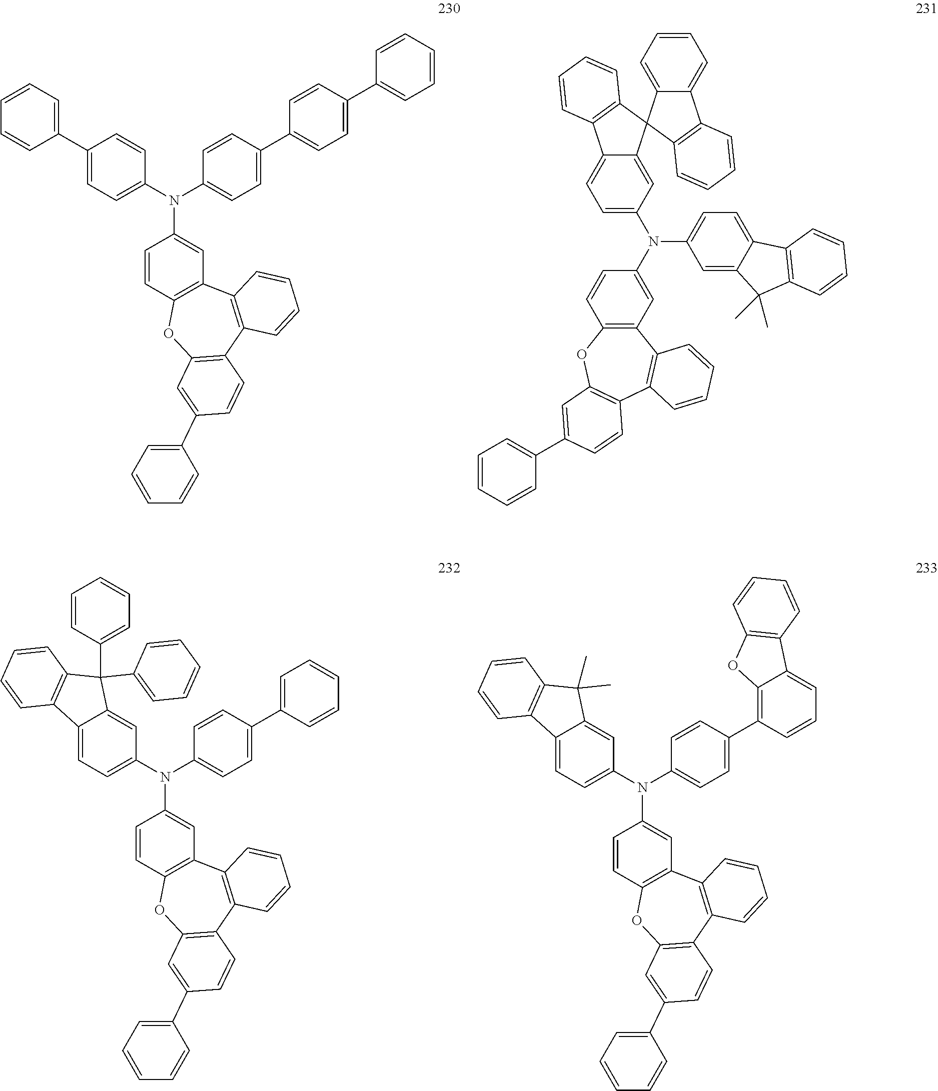 [SMILES: c1ccc(-c2ccc(-c3ccc(N(c4ccc(-c5ccccc5)cc4)c4ccc5c(c4)-c4ccccc4-c4ccc(-c6ccccc6)cc4O5)cc3)cc2)cc1 ; CC1(C)c2ccccc2-c2ccc(N(c3ccc4c(c3)-c3ccccc3-c3ccc(-c5ccccc5)cc3O4)c3ccc4c(c3)C3(c5ccccc5-c5ccccc53)c3ccccc3-4)cc21 ; c1ccc(-c2ccc(N(c3ccc4c(c3)-c3ccccc3-c3ccc(-c5ccccc5)cc3O4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)-c3ccccc3-c3ccc(-c5ccccc5)cc3O4)cc21]

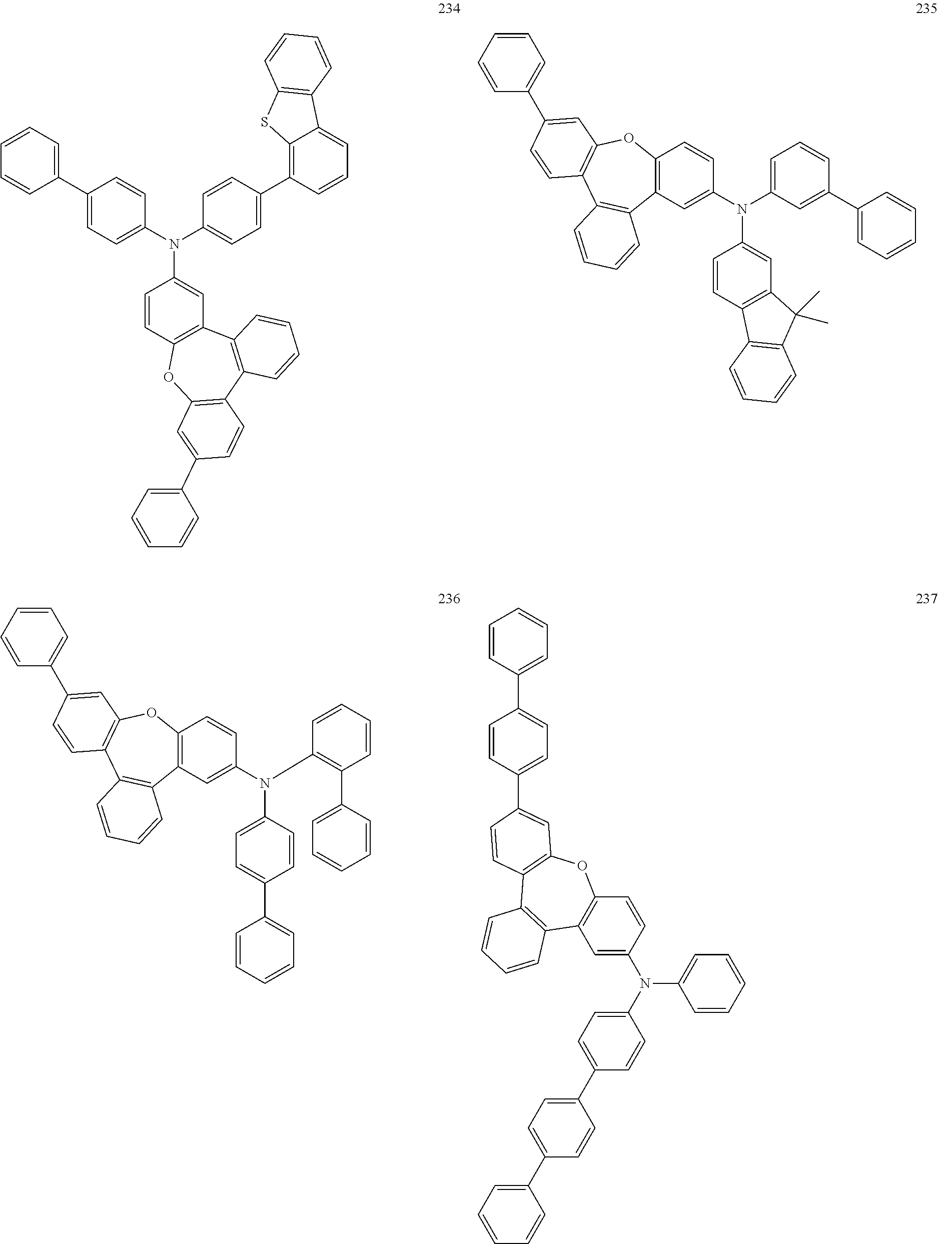 [SMILES: c1ccc(-c2ccc(N(c3ccc(-c4cccc5c4sc4ccccc45)cc3)c3ccc4c(c3)-c3ccccc3-c3ccc(-c5ccccc5)cc3O4)cc2)cc1 ; CC1(C)c2ccccc2-c2ccc(N(c3cccc(-c4ccccc4)c3)c3ccc4c(c3)-c3ccccc3-c3ccc(-c5ccccc5)cc3O4)cc21 ; c1ccc(-c2ccc(N(c3ccc4c(c3)-c3ccccc3-c3ccc(-c5ccccc5)cc3O4)c3ccccc3-c3ccccc3)cc2)cc1 ; c1ccc(-c2ccc(-c3ccc(N(c4ccccc4)c4ccc5c(c4)-c4ccccc4-c4ccc(-c6ccc(-c7ccccc7)cc6)cc4O5)cc3)cc2)cc1]

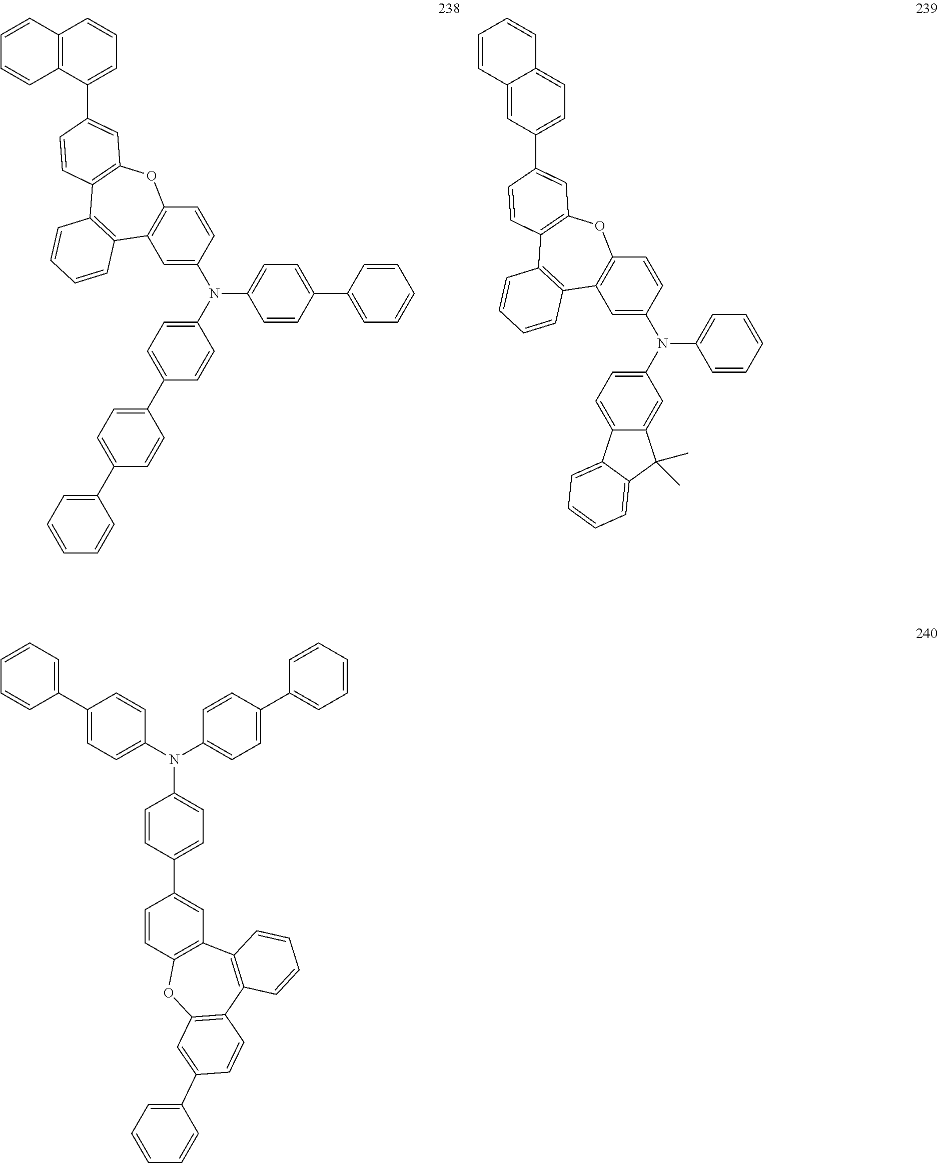 [SMILES: c1ccc(-c2ccc(-c3ccc(N(c4ccc(-c5ccccc5)cc4)c4ccc5c(c4)-c4ccccc4-c4ccc(-c6cccc7ccccc67)cc4O5)cc3)cc2)cc1 ; CC1(C)c2ccccc2-c2ccc(N(c3ccccc3)c3ccc4c(c3)-c3ccccc3-c3ccc(-c5ccc6ccccc6c5)cc3O4)cc21 ; c1ccc(-c2ccc(N(c3ccc(-c4ccccc4)cc3)c3ccc(-c4ccc5c(c4)-c4ccccc4-c4ccc(-c6ccccc6)cc4O5)cc3)cc2)cc1]

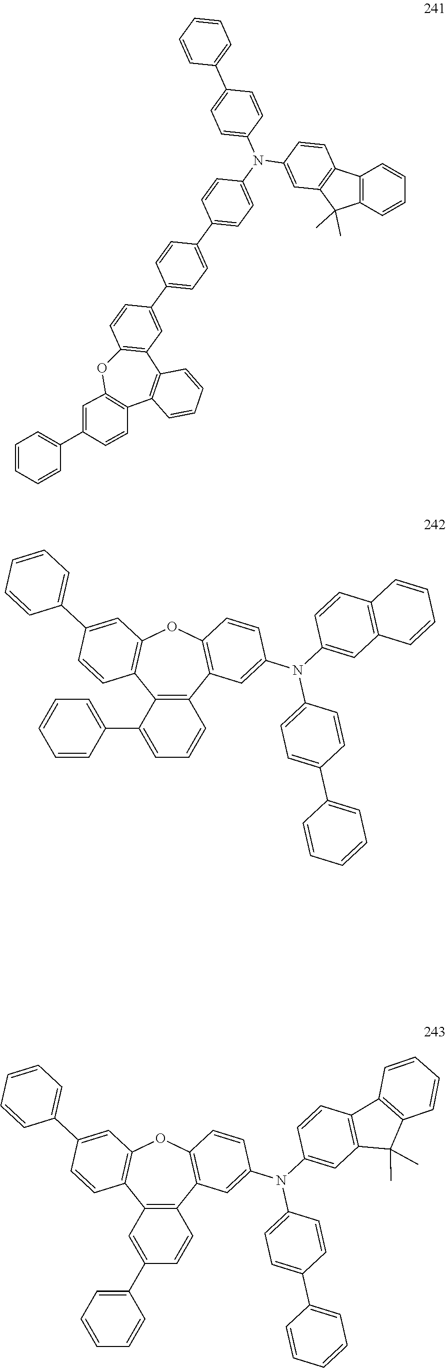 [SMILES: CC1(C)c2ccccc2-c2ccc(N(c3ccc(-c4ccccc4)cc3)c3ccc(-c4ccc(-c5ccc6c(c5)-c5ccccc5-c5ccc(-c7ccccc7)cc5O6)cc4)cc3)cc21 ; c1ccc(-c2ccc(N(c3ccc4c(c3)-c3cccc(-c5ccccc5)c3-c3ccc(-c5ccccc5)cc3O4)c3ccc4ccccc4c3)cc2)cc1 ; CC1(C)c2ccccc2-c2ccc(N(c3ccc(-c4ccccc4)cc3)c3ccc4c(c3)-c3ccc(-c5ccccc5)cc3-c3ccc(-c5ccccc5)cc3O4)cc21]

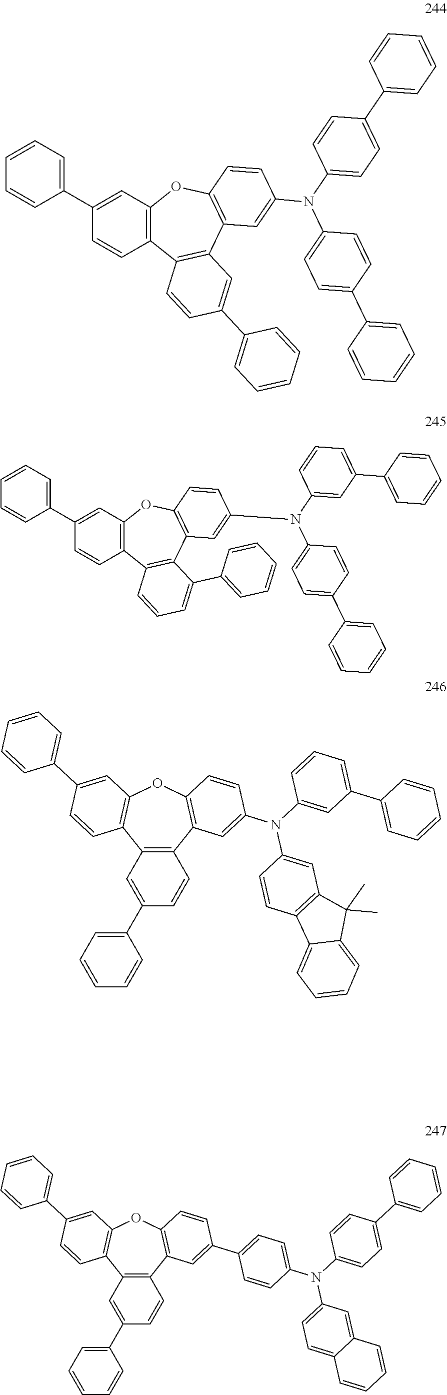 [SMILES: c1ccc(-c2ccc(N(c3ccc(-c4ccccc4)cc3)c3ccc4c(c3)-c3cc(-c5ccccc5)ccc3-c3ccc(-c5ccccc5)cc3O4)cc2)cc1 ; c1ccc(-c2ccc(N(c3cccc(-c4ccccc4)c3)c3ccc4c(c3)-c3c(-c5ccccc5)cccc3-c3ccc(-c5ccccc5)cc3O4)cc2)cc1 ; CC1(C)c2ccccc2-c2ccc(N(c3cccc(-c4ccccc4)c3)c3ccc4c(c3)-c3ccc(-c5ccccc5)cc3-c3ccc(-c5ccccc5)cc3O4)cc21 ; c1ccc(-c2ccc(N(c3ccc(-c4ccc5c(c4)-c4ccc(-c6ccccc6)cc4-c4ccc(-c6ccccc6)cc4O5)cc3)c3ccc4ccccc4c3)cc2)cc1]

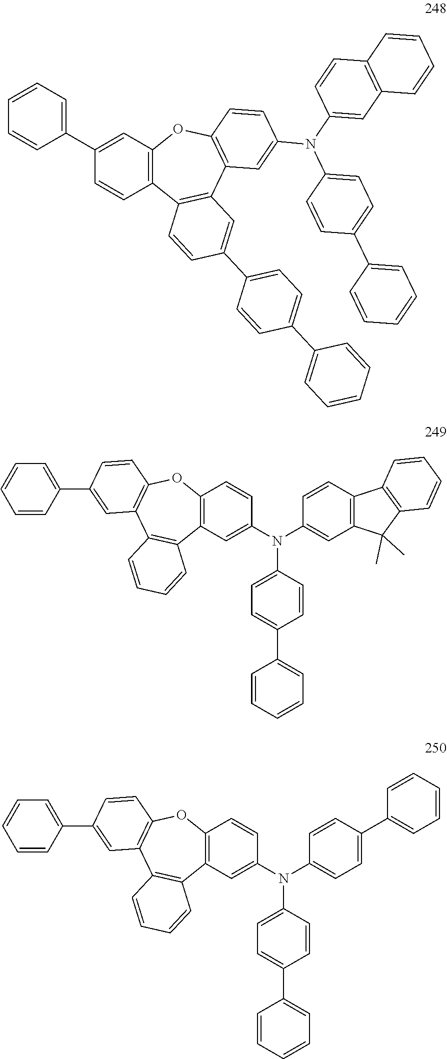 [SMILES: c1ccc(-c2ccc(-c3ccc4c(c3)-c3cc(N(c5ccc(-c6ccccc6)cc5)c5ccc6ccccc6c5)ccc3Oc3cc(-c5ccccc5)ccc3-4)cc2)cc1 ; CC1(C)c2ccccc2-c2ccc(N(c3ccc(-c4ccccc4)cc3)c3ccc4c(c3)-c3ccccc3-c3cc(-c5ccccc5)ccc3O4)cc21 ; c1ccc(-c2ccc(N(c3ccc(-c4ccccc4)cc3)c3ccc4c(c3)-c3ccccc3-c3cc(-c5ccccc5)ccc3O4)cc2)cc1]

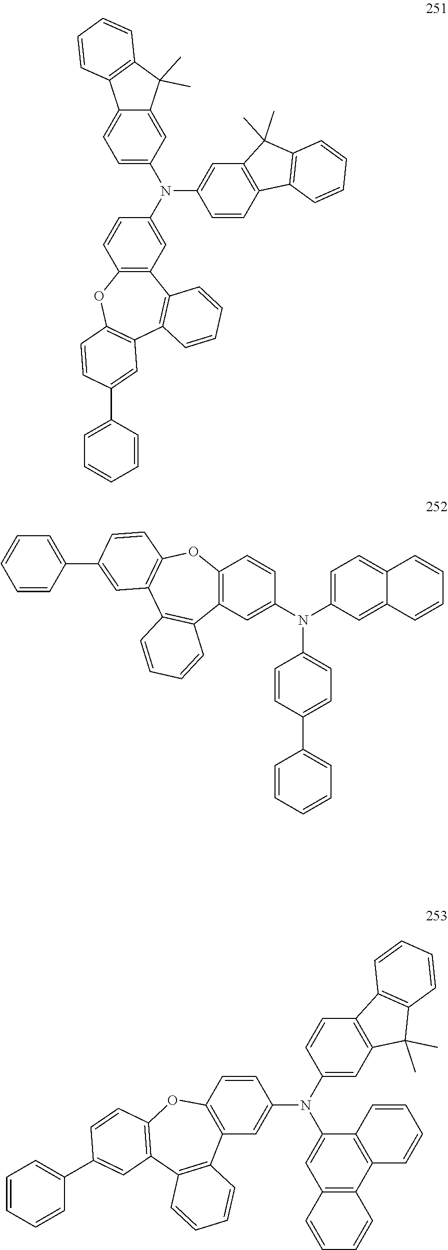 [SMILES: CC1(C)c2ccccc2-c2ccc(N(c3ccc4c(c3)-c3ccccc3-c3cc(-c5ccccc5)ccc3O4)c3ccc4c(c3)C(C)(C)c3ccccc3-4)cc21 ; c1ccc(-c2ccc(N(c3ccc4c(c3)-c3ccccc3-c3cc(-c5ccccc5)ccc3O4)c3ccc4ccccc4c3)cc2)cc1 ; CC1(C)c2ccccc2-c2ccc(N(c3ccc4c(c3)-c3ccccc3-c3cc(-c5ccccc5)ccc3O4)c3cc4ccccc4c4ccccc34)cc21]

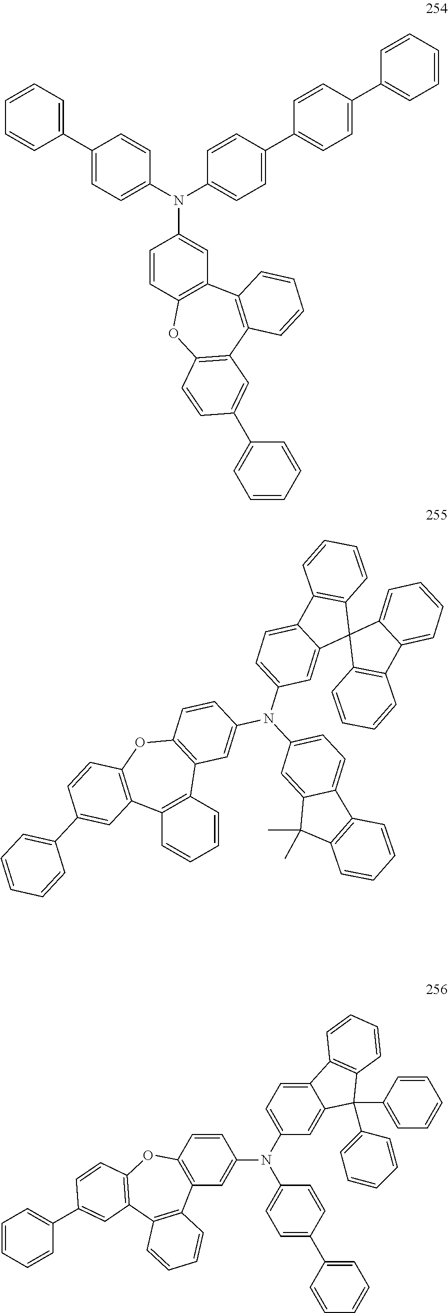 [SMILES: c1ccc(-c2ccc(-c3ccc(N(c4ccc(-c5ccccc5)cc4)c4ccc5c(c4)-c4ccccc4-c4cc(-c6ccccc6)ccc4O5)cc3)cc2)cc1 ; CC1(C)c2ccccc2-c2ccc(N(c3ccc4c(c3)-c3ccccc3-c3cc(-c5ccccc5)ccc3O4)c3ccc4c(c3)C3(c5ccccc5-c5ccccc53)c3ccccc3-4)cc21 ; c1ccc(-c2ccc(N(c3ccc4c(c3)-c3ccccc3-c3cc(-c5ccccc5)ccc3O4)c3ccc4c(c3)C(c3ccccc3)(c3ccccc3)c3ccccc3-4)cc2)cc1]

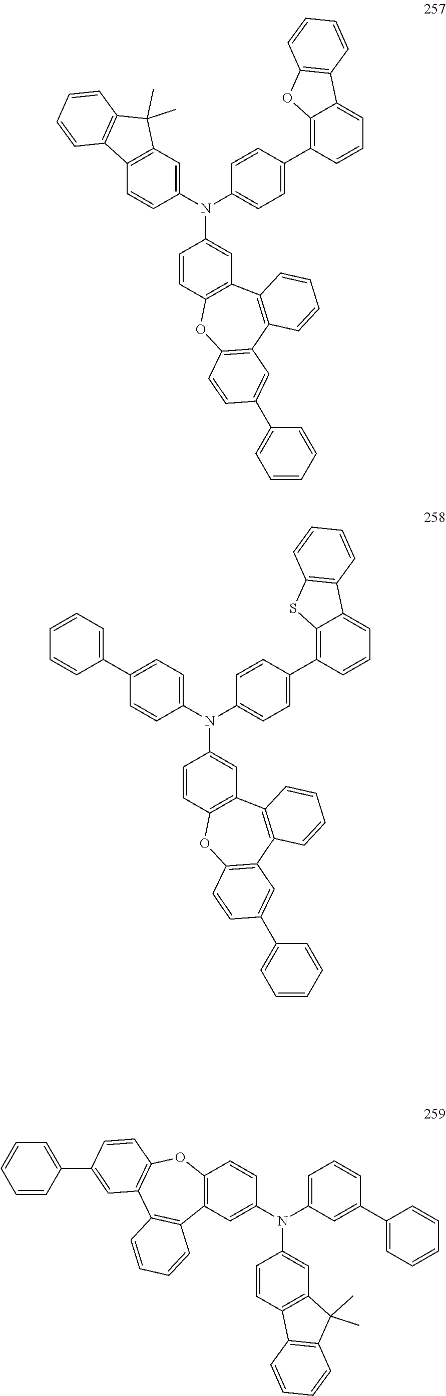 [SMILES: CC1(C)c2ccccc2-c2ccc(N(c3ccc(-c4cccc5c4oc4ccccc45)cc3)c3ccc4c(c3)-c3ccccc3-c3cc(-c5ccccc5)ccc3O4)cc21 ; c1ccc(-c2ccc(N(c3ccc(-c4cccc5c4sc4ccccc45)cc3)c3ccc4c(c3)-c3ccccc3-c3cc(-c5ccccc5)ccc3O4)cc2)cc1 ; CC1(C)c2ccccc2-c2ccc(N(c3cccc(-c4ccccc4)c3)c3ccc4c(c3)-c3ccccc3-c3cc(-c5ccccc5)ccc3O4)cc21]

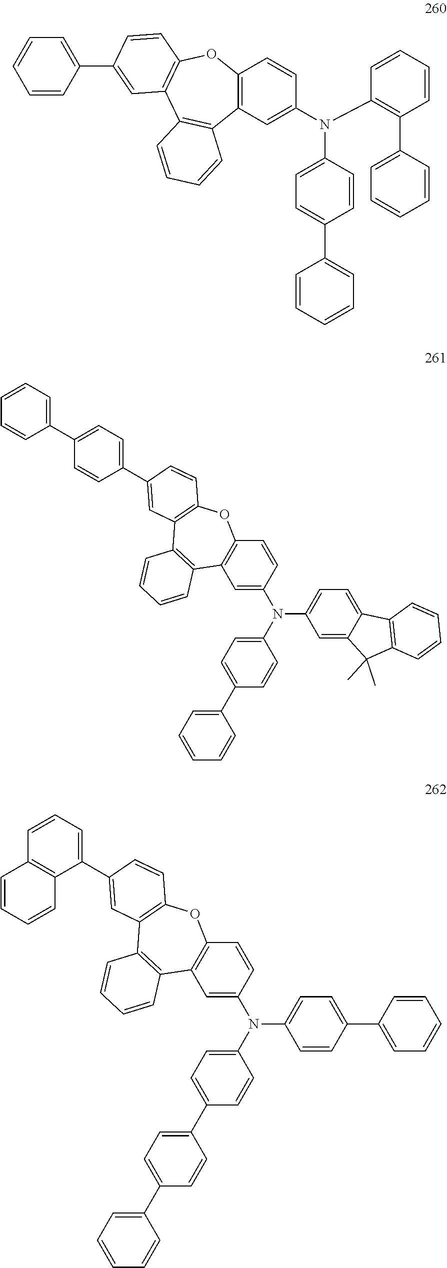 [SMILES: c1ccc(-c2ccc(N(c3ccc4c(c3)-c3ccccc3-c3cc(-c5ccccc5)ccc3O4)c3ccccc3-c3ccccc3)cc2)cc1 ; CC1(C)c2ccccc2-c2ccc(N(c3ccc(-c4ccccc4)cc3)c3ccc4c(c3)-c3ccccc3-c3cc(-c5ccc(-c6ccccc6)cc5)ccc3O4)cc21 ; c1ccc(-c2ccc(-c3ccc(N(c4ccc(-c5ccccc5)cc4)c4ccc5c(c4)-c4ccccc4-c4cc(-c6cccc7ccccc67)ccc4O5)cc3)cc2)cc1]

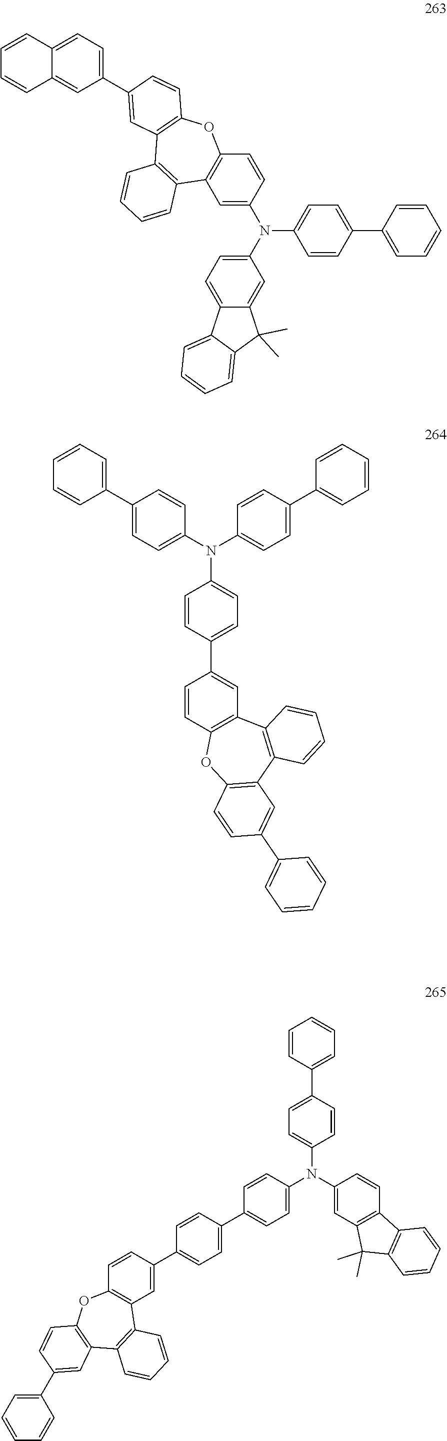 [SMILES: CC1(C)c2ccccc2-c2ccc(N(c3ccc(-c4ccccc4)cc3)c3ccc4c(c3)-c3ccccc3-c3cc(-c5ccc6ccccc6c5)ccc3O4)cc21 ; c1ccc(-c2ccc(N(c3ccc(-c4ccccc4)cc3)c3ccc(-c4ccc5c(c4)-c4ccccc4-c4cc(-c6ccccc6)ccc4O5)cc3)cc2)cc1 ; CC1(C)c2ccccc2-c2ccc(N(c3ccc(-c4ccccc4)cc3)c3ccc(-c4ccc(-c5ccc6c(c5)-c5ccccc5-c5cc(-c7ccccc7)ccc5O6)cc4)cc3)cc21]

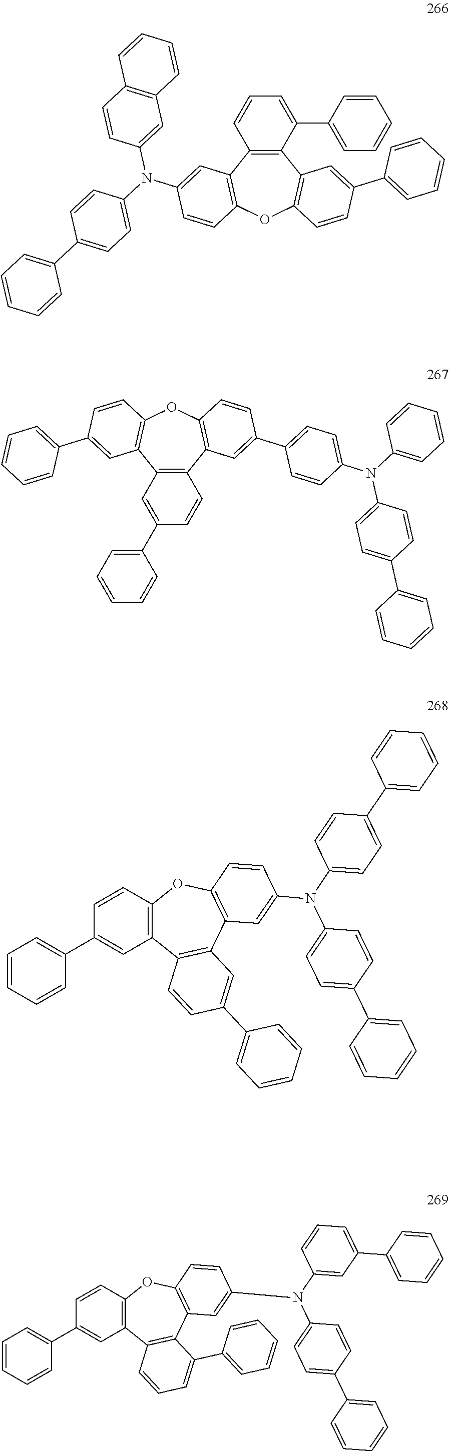 [SMILES: c1ccc(-c2ccc(N(c3ccc4c(c3)-c3cccc(-c5ccccc5)c3-c3cc(-c5ccccc5)ccc3O4)c3ccc4ccccc4c3)cc2)cc1 ; c1ccc(-c2ccc(N(c3ccccc3)c3ccc(-c4ccc5c(c4)-c4ccc(-c6ccccc6)cc4-c4cc(-c6ccccc6)ccc4O5)cc3)cc2)cc1 ; c1ccc(-c2ccc(N(c3ccc(-c4ccccc4)cc3)c3ccc4c(c3)-c3cc(-c5ccccc5)ccc3-c3cc(-c5ccccc5)ccc3O4)cc2)cc1 ; c1ccc(-c2ccc(N(c3cccc(-c4ccccc4)c3)c3ccc4c(c3)-c3c(-c5ccccc5)cccc3-c3cc(-c5ccccc5)ccc3O4)cc2)cc1]

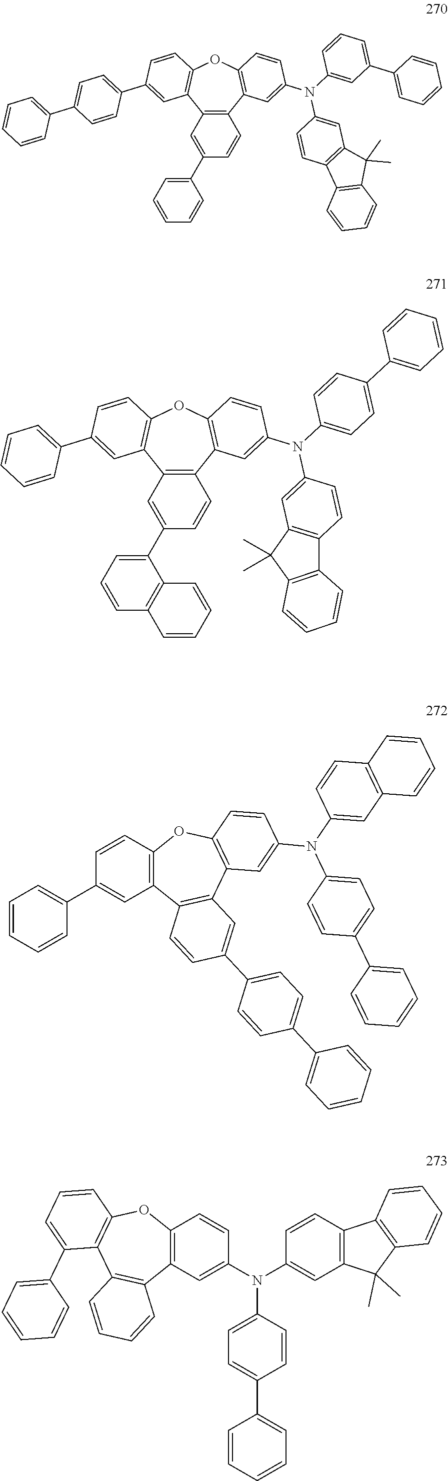 [SMILES: CC1(C)c2ccccc2-c2ccc(N(c3cccc(-c4ccccc4)c3)c3ccc4c(c3)-c3ccc(-c5ccccc5)cc3-c3cc(-c5ccc(-c6ccccc6)cc5)ccc3O4)cc21 ; CC1(C)c2ccccc2-c2ccc(N(c3ccc(-c4ccccc4)cc3)c3ccc4c(c3)-c3ccc(-c5cccc6ccccc56)cc3-c3cc(-c5ccccc5)ccc3O4)cc21 ; c1ccc(-c2ccc(-c3ccc4c(c3)-c3cc(N(c5ccc(-c6ccccc6)cc5)c5ccc6ccccc6c5)ccc3Oc3ccc(-c5ccccc5)cc3-4)cc2)cc1 ; CC1(C)c2ccccc2-c2ccc(N(c3ccc(-c4ccccc4)cc3)c3ccc4c(c3)-c3ccccc3-c3c(cccc3-c3ccccc3)O4)cc21]

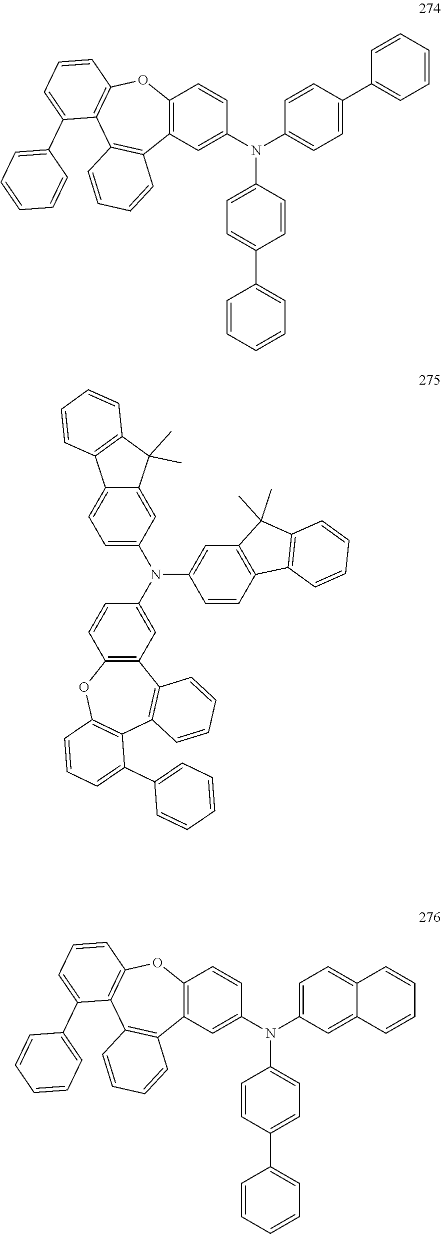 [SMILES: c1ccc(-c2ccc(N(c3ccc(-c4ccccc4)cc3)c3ccc4c(c3)-c3ccccc3-c3c(cccc3-c3ccccc3)O4)cc2)cc1 ; CC1(C)c2ccccc2-c2ccc(N(c3ccc4c(c3)-c3ccccc3-c3c(cccc3-c3ccccc3)O4)c3ccc4c(c3)C(C)(C)c3ccccc3-4)cc21 ; c1ccc(-c2ccc(N(c3ccc4c(c3)-c3ccccc3-c3c(cccc3-c3ccccc3)O4)c3ccc4ccccc4c3)cc2)cc1]

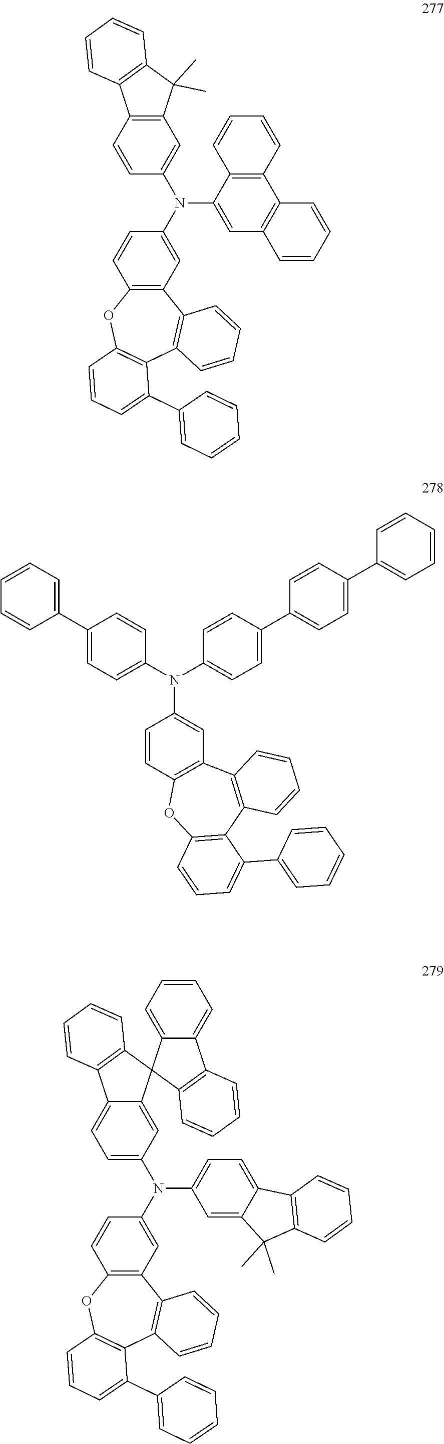 [SMILES: CC1(C)c2ccccc2-c2ccc(N(c3ccc4c(c3)-c3ccccc3-c3c(cccc3-c3ccccc3)O4)c3cc4ccccc4c4ccccc34)cc21 ; c1ccc(-c2ccc(-c3ccc(N(c4ccc(-c5ccccc5)cc4)c4ccc5c(c4)-c4ccccc4-c4c(cccc4-c4ccccc4)O5)cc3)cc2)cc1 ; CC1(C)c2ccccc2-c2ccc(N(c3ccc4c(c3)-c3ccccc3-c3c(cccc3-c3ccccc3)O4)c3ccc4c(c3)C3(c5ccccc5-c5ccccc53)c3ccccc3-4)cc21]

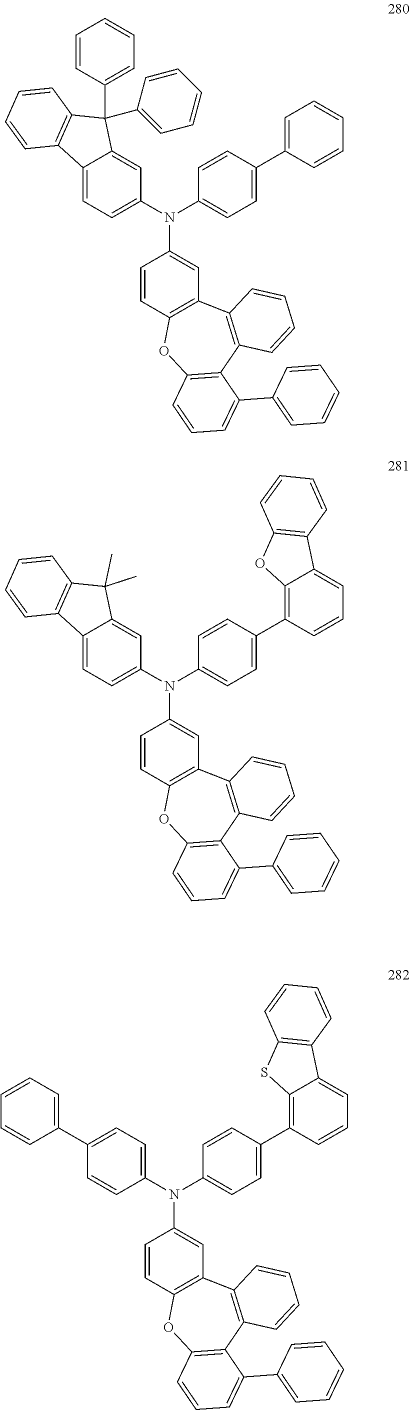 [SMILES: c1ccc(-c2ccc(N(c3ccc4c(c3)-c3ccccc3-c3c(cccc3-c3ccccc3)O4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)-c3ccccc3-c3c(cccc3-c3ccccc3)O4)cc21 ; c1ccc(-c2ccc(N(c3ccc(-c4cccc5c4sc4ccccc45)cc3)c3ccc4c(c3)-c3ccccc3-c3c(cccc3-c3ccccc3)O4)cc2)cc1]

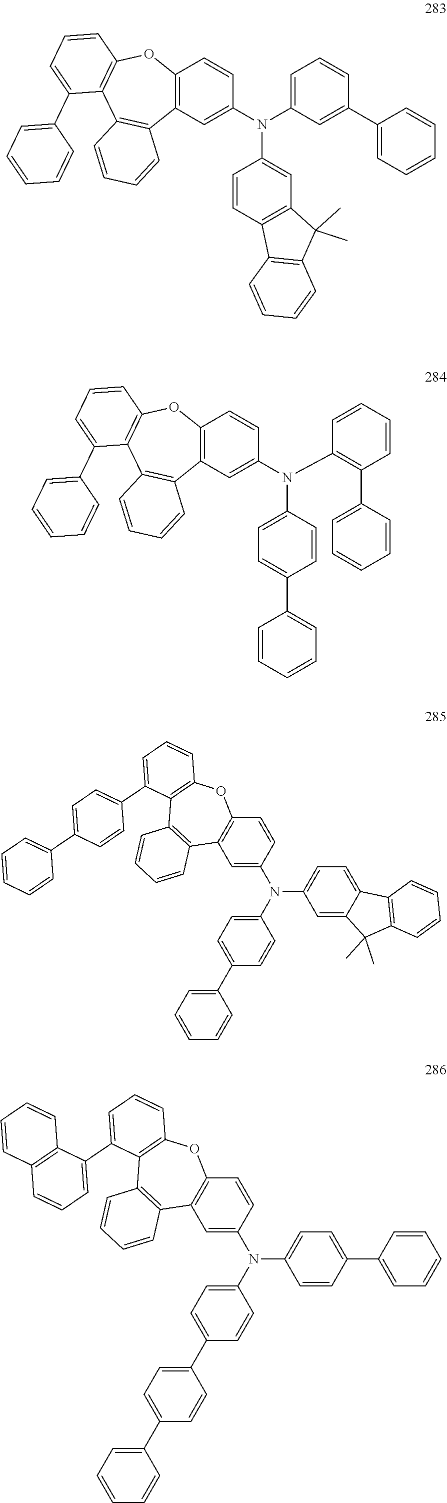 [SMILES: CC1(C)c2ccccc2-c2ccc(N(c3cccc(-c4ccccc4)c3)c3ccc4c(c3)-c3ccccc3-c3c(cccc3-c3ccccc3)O4)cc21 ; c1ccc(-c2ccc(N(c3ccc4c(c3)-c3ccccc3-c3c(cccc3-c3ccccc3)O4)c3ccccc3-c3ccccc3)cc2)cc1 ; CC1(C)c2ccccc2-c2ccc(N(c3ccc(-c4ccccc4)cc3)c3ccc4c(c3)-c3ccccc3-c3c(cccc3-c3ccc(-c5ccccc5)cc3)O4)cc21 ; c1ccc(-c2ccc(-c3ccc(N(c4ccc(-c5ccccc5)cc4)c4ccc5c(c4)-c4ccccc4-c4c(cccc4-c4cccc6ccccc46)O5)cc3)cc2)cc1]

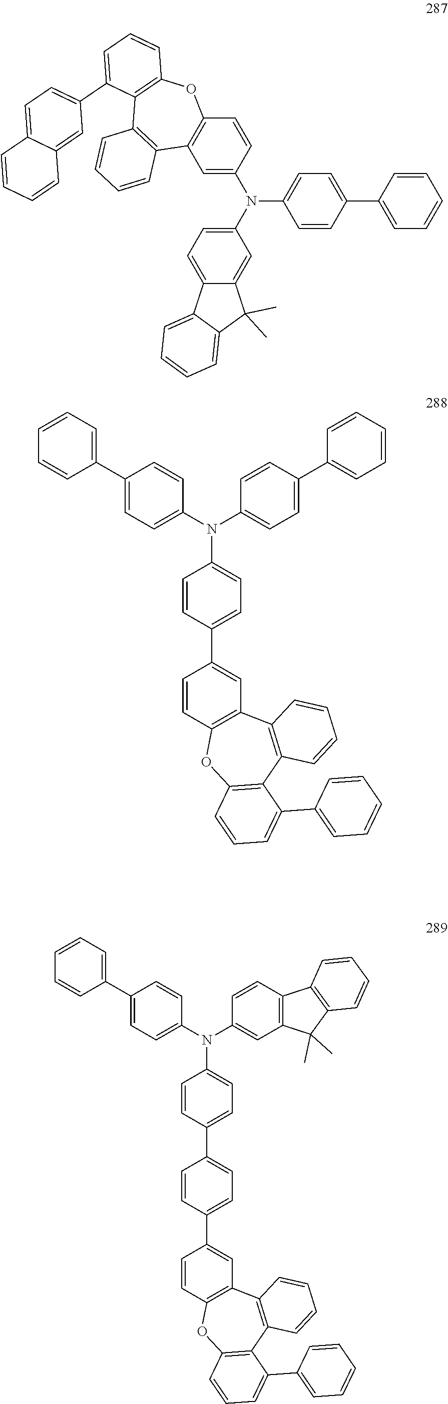 [SMILES: CC1(C)c2ccccc2-c2ccc(N(c3ccc(-c4ccccc4)cc3)c3ccc4c(c3)-c3ccccc3-c3c(cccc3-c3ccc5ccccc5c3)O4)cc21 ; c1ccc(-c2ccc(N(c3ccc(-c4ccccc4)cc3)c3ccc(-c4ccc5c(c4)-c4ccccc4-c4c(cccc4-c4ccccc4)O5)cc3)cc2)cc1 ; CC1(C)c2ccccc2-c2ccc(N(c3ccc(-c4ccccc4)cc3)c3ccc(-c4ccc(-c5ccc6c(c5)-c5ccccc5-c5c(cccc5-c5ccccc5)O6)cc4)cc3)cc21]

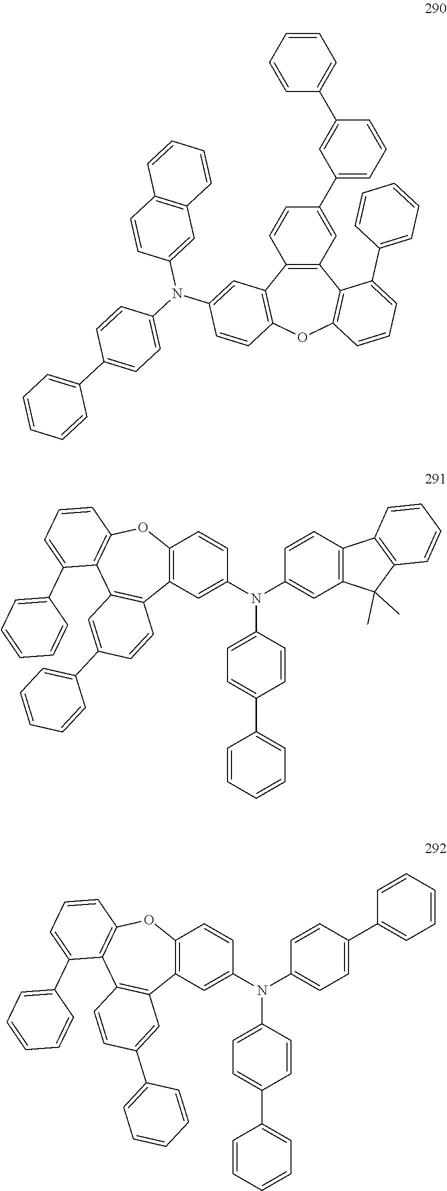 [SMILES: c1ccc(-c2ccc(N(c3ccc4c(c3)-c3ccc(-c5cccc(-c6ccccc6)c5)cc3-c3c(cccc3-c3ccccc3)O4)c3ccc4ccccc4c3)cc2)cc1 ; CC1(C)c2ccccc2-c2ccc(N(c3ccc(-c4ccccc4)cc3)c3ccc4c(c3)-c3ccc(-c5ccccc5)cc3-c3c(cccc3-c3ccccc3)O4)cc21 ; c1ccc(-c2ccc(N(c3ccc(-c4ccccc4)cc3)c3ccc4c(c3)-c3cc(-c5ccccc5)ccc3-c3c(cccc3-c3ccccc3)O4)cc2)cc1]

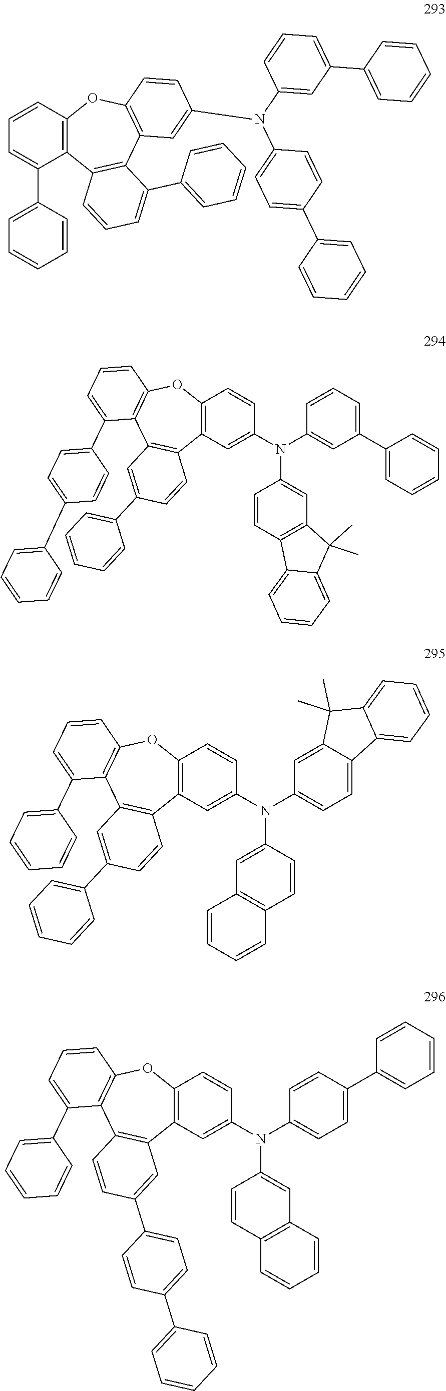 [SMILES: c1ccc(-c2ccc(N(c3cccc(-c4ccccc4)c3)c3ccc4c(c3)-c3c(-c5ccccc5)cccc3-c3c(cccc3-c3ccccc3)O4)cc2)cc1 ; CC1(C)c2ccccc2-c2ccc(N(c3cccc(-c4ccccc4)c3)c3ccc4c(c3)-c3ccc(-c5ccccc5)cc3-c3c(cccc3-c3ccc(-c5ccccc5)cc3)O4)cc21 ; CC1(C)c2ccccc2-c2ccc(N(c3ccc4c(c3)-c3ccc(-c5ccccc5)cc3-c3c(cccc3-c3ccccc3)O4)c3ccc4ccccc4c3)cc21 ; c1ccc(-c2ccc(-c3ccc4c(c3)-c3cc(N(c5ccc(-c6ccccc6)cc5)c5ccc6ccccc6c5)ccc3Oc3cccc(-c5ccccc5)c3-4)cc2)cc1]

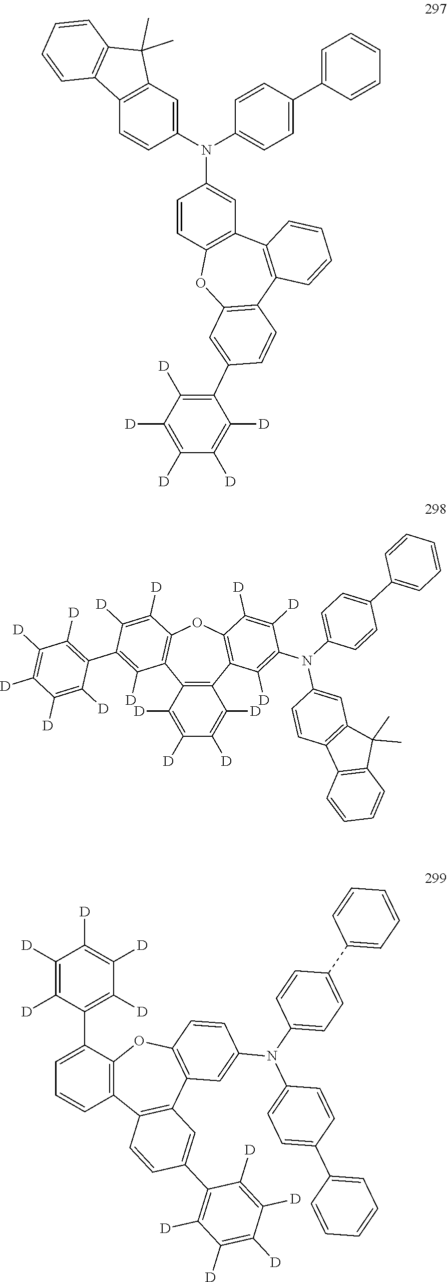

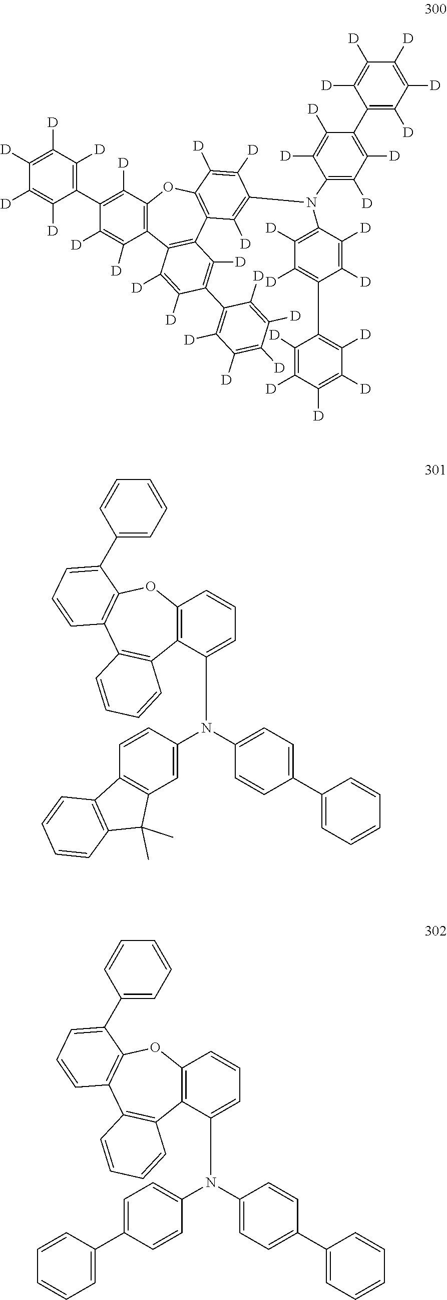 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])-c3c([2H])c(-c5c([2H])c([2H])c([2H])c([2H])c5[2H])c([2H])c([2H])c3-c3c([2H])c([2H])c(-c5c([2H])c([2H])c([2H])c([2H])c5[2H])c([2H])c3O4)c([2H])c2[2H])c([2H])c1[2H] ; CC1(C)c2ccccc2-c2ccc(N(c3ccc(-c4ccccc4)cc3)c3cccc4c3-c3ccccc3-c3cccc(-c5ccccc5)c3O4)cc21 ; c1ccc(-c2ccc(N(c3ccc(-c4ccccc4)cc3)c3cccc4c3-c3ccccc3-c3cccc(-c5ccccc5)c3O4)cc2)cc1]

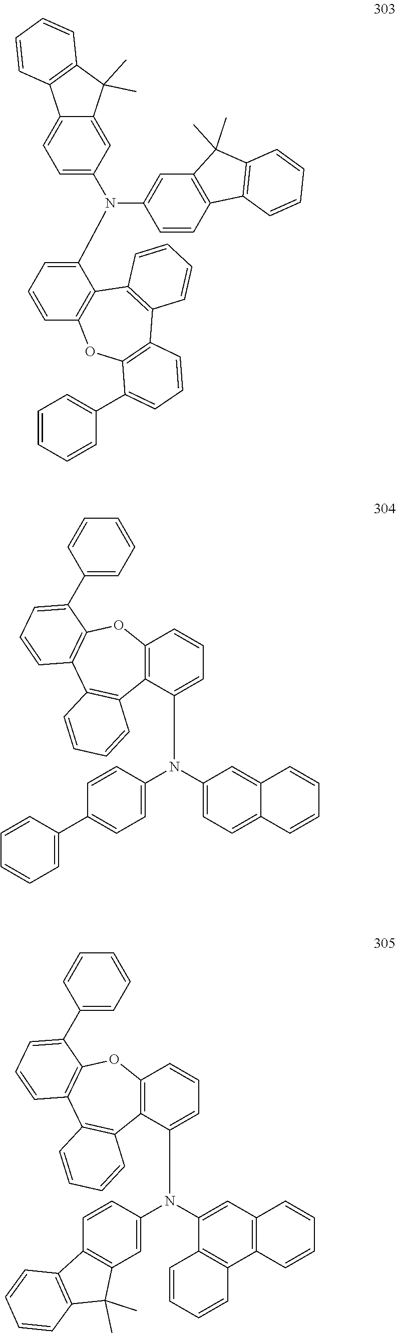 [SMILES: CC1(C)c2ccccc2-c2ccc(N(c3ccc4c(c3)C(C)(C)c3ccccc3-4)c3cccc4c3-c3ccccc3-c3cccc(-c5ccccc5)c3O4)cc21 ; c1ccc(-c2ccc(N(c3ccc4ccccc4c3)c3cccc4c3-c3ccccc3-c3cccc(-c5ccccc5)c3O4)cc2)cc1 ; CC1(C)c2ccccc2-c2ccc(N(c3cccc4c3-c3ccccc3-c3cccc(-c5ccccc5)c3O4)c3cc4ccccc4c4ccccc34)cc21]

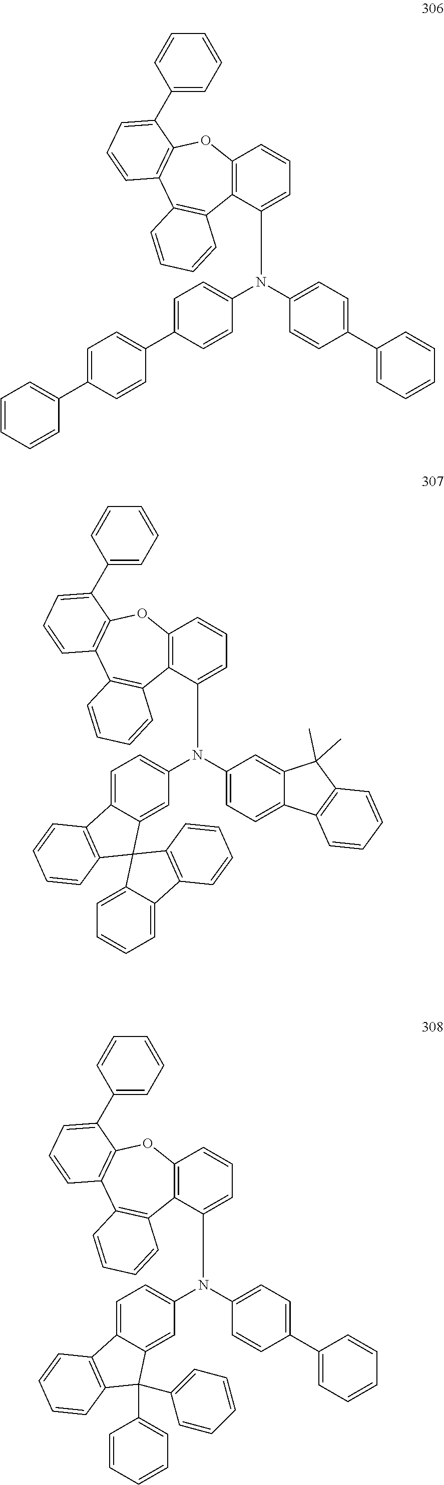 [SMILES: c1ccc(-c2ccc(-c3ccc(N(c4ccc(-c5ccccc5)cc4)c4cccc5c4-c4ccccc4-c4cccc(-c6ccccc6)c4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3ccccc3-c3cccc(-c5ccccc5)c3O4)cc21 ; c1ccc(-c2ccc(N(c3ccc4c(c3)C(c3ccccc3)(c3ccccc3)c3ccccc3-4)c3cccc4c3-c3ccccc3-c3cccc(-c5ccccc5)c3O4)cc2)cc1]

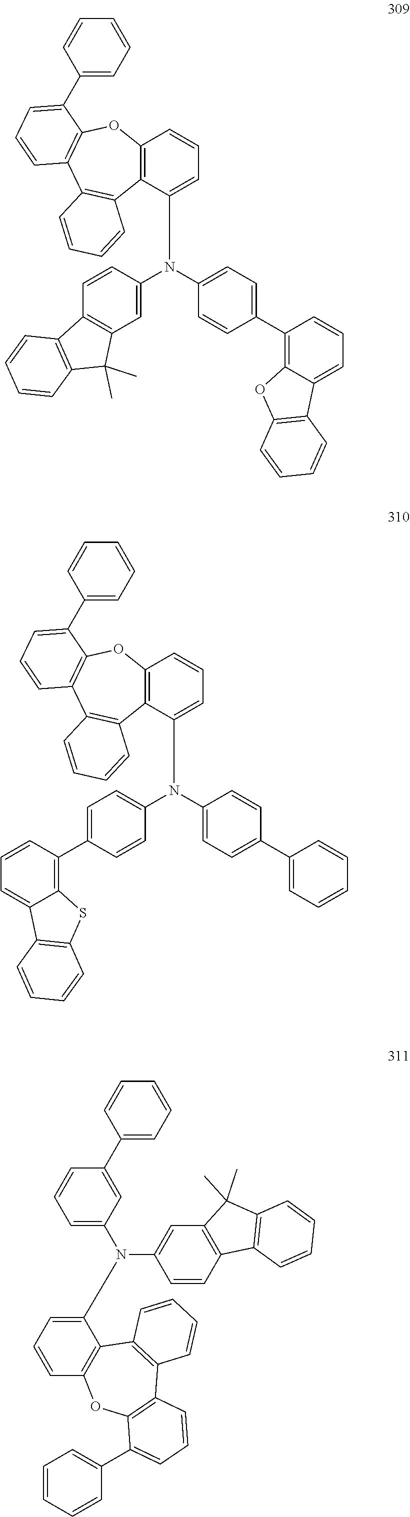 [SMILES: CC1(C)c2ccccc2-c2ccc(N(c3ccc(-c4cccc5c4oc4ccccc45)cc3)c3cccc4c3-c3ccccc3-c3cccc(-c5ccccc5)c3O4)cc21 ; c1ccc(-c2ccc(N(c3ccc(-c4cccc5c4sc4ccccc45)cc3)c3cccc4c3-c3ccccc3-c3cccc(-c5ccccc5)c3O4)cc2)cc1 ; CC1(C)c2ccccc2-c2ccc(N(c3cccc(-c4ccccc4)c3)c3cccc4c3-c3ccccc3-c3cccc(-c5ccccc5)c3O4)cc21]

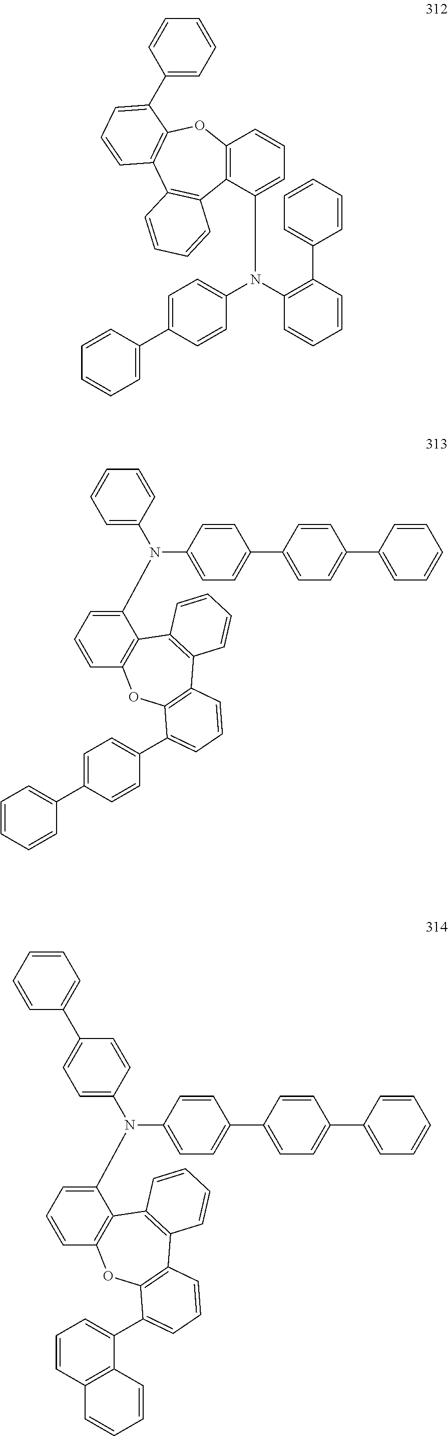 [SMILES: c1ccc(-c2ccc(N(c3ccccc3-c3ccccc3)c3cccc4c3-c3ccccc3-c3cccc(-c5ccccc5)c3O4)cc2)cc1 ; c1ccc(-c2ccc(-c3ccc(N(c4ccccc4)c4cccc5c4-c4ccccc4-c4cccc(-c6ccc(-c7ccccc7)cc6)c4O5)cc3)cc2)cc1 ; c1ccc(-c2ccc(-c3ccc(N(c4ccc(-c5ccccc5)cc4)c4cccc5c4-c4ccccc4-c4cccc(-c6cccc7ccccc67)c4O5)cc3)cc2)cc1]

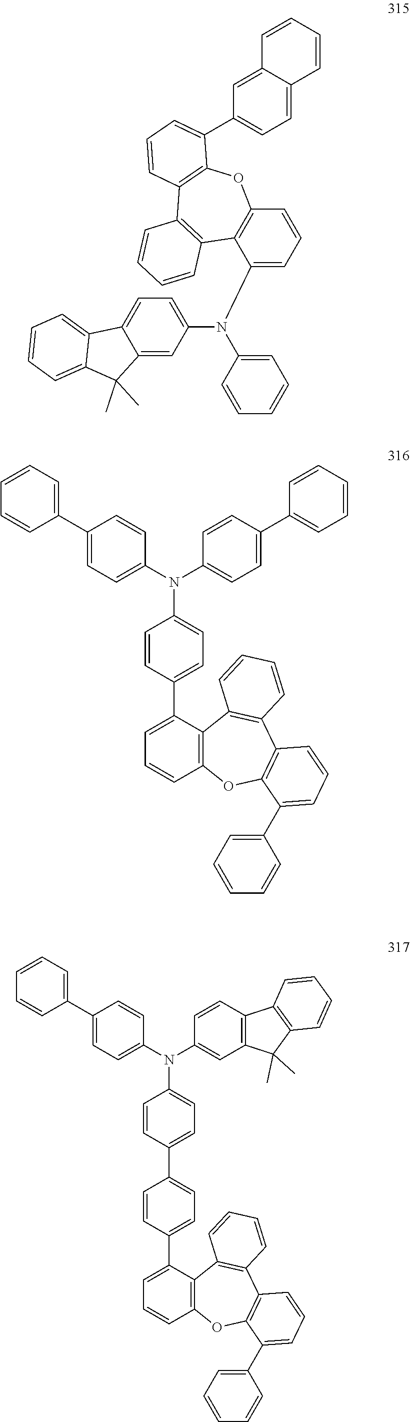 [SMILES: CC1(C)c2ccccc2-c2ccc(N(c3ccccc3)c3cccc4c3-c3ccccc3-c3cccc(-c5ccc6ccccc6c5)c3O4)cc21 ; c1ccc(-c2ccc(N(c3ccc(-c4ccccc4)cc3)c3ccc(-c4cccc5c4-c4ccccc4-c4cccc(-c6ccccc6)c4O5)cc3)cc2)cc1 ; CC1(C)c2ccccc2-c2ccc(N(c3ccc(-c4ccccc4)cc3)c3ccc(-c4ccc(-c5cccc6c5-c5ccccc5-c5cccc(-c7ccccc7)c5O6)cc4)cc3)cc21]

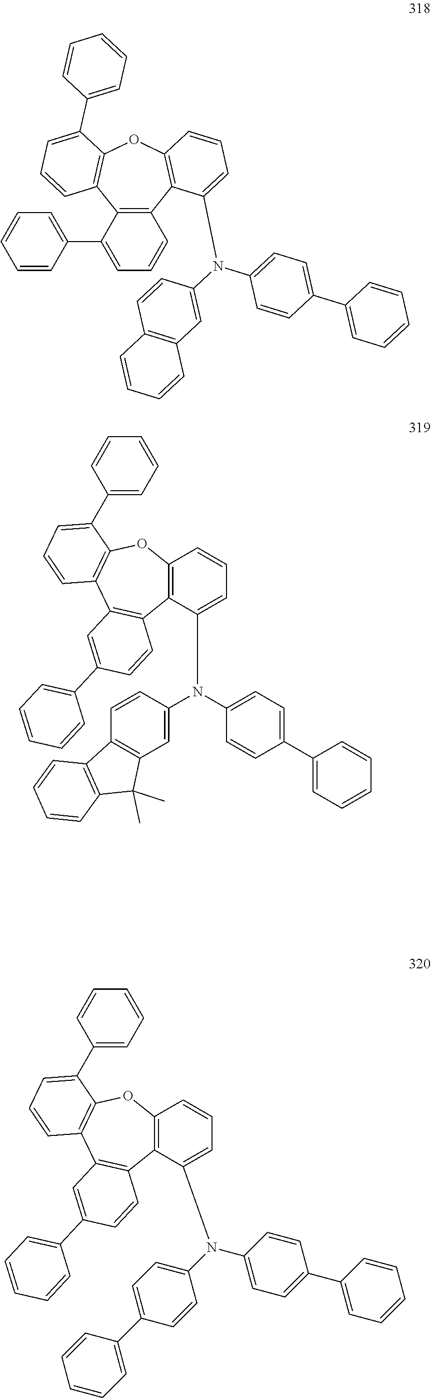

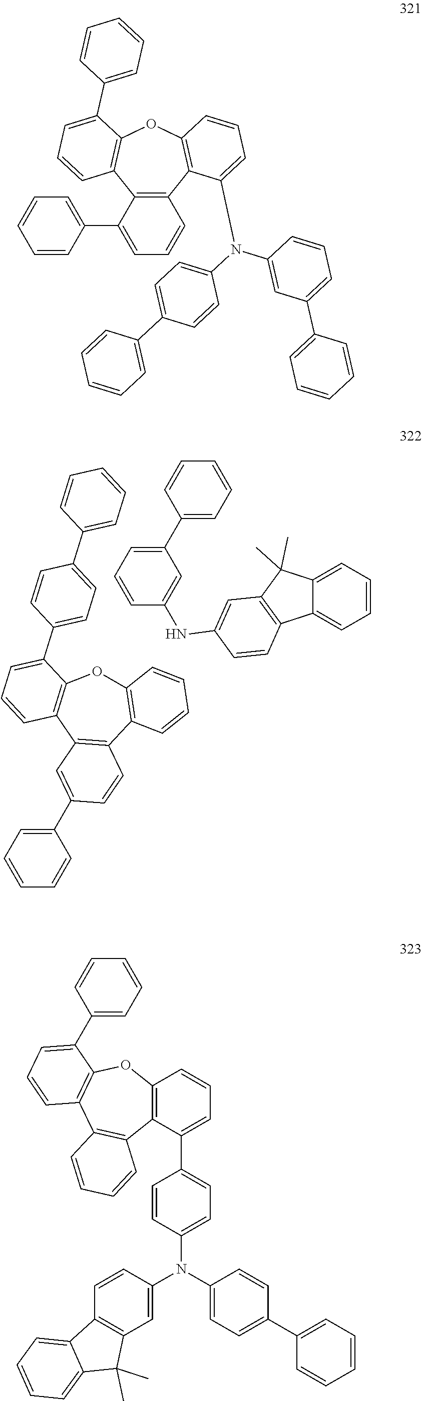

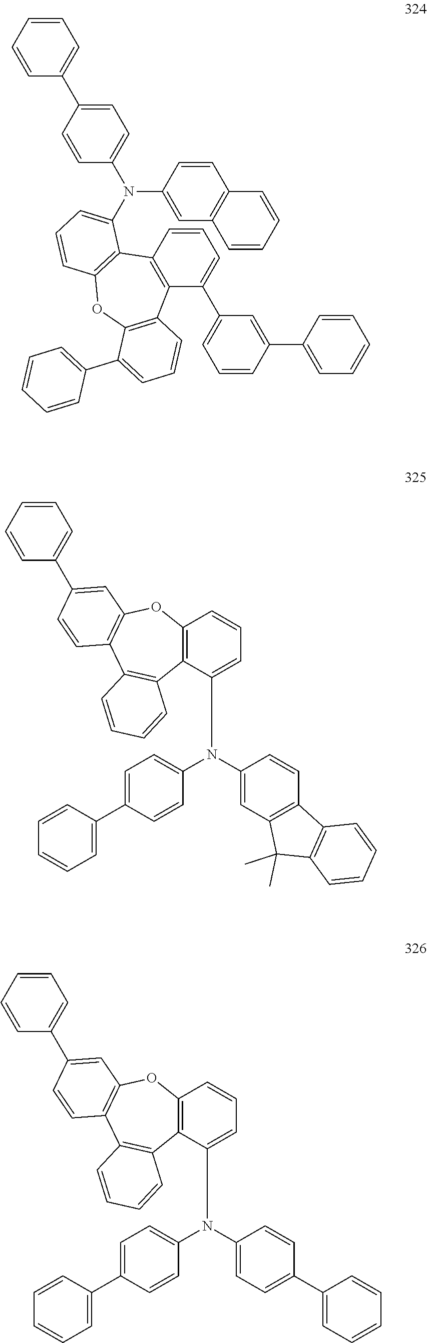 [SMILES: c1ccc(-c2ccc(N(c3ccc4ccccc4c3)c3cccc4c3-c3cccc(-c5cccc(-c6ccccc6)c5)c3-c3cccc(-c5ccccc5)c3O4)cc2)cc1 ; CC1(C)c2ccccc2-c2ccc(N(c3ccc(-c4ccccc4)cc3)c3cccc4c3-c3ccccc3-c3ccc(-c5ccccc5)cc3O4)cc21 ; c1ccc(-c2ccc(N(c3ccc(-c4ccccc4)cc3)c3cccc4c3-c3ccccc3-c3ccc(-c5ccccc5)cc3O4)cc2)cc1]

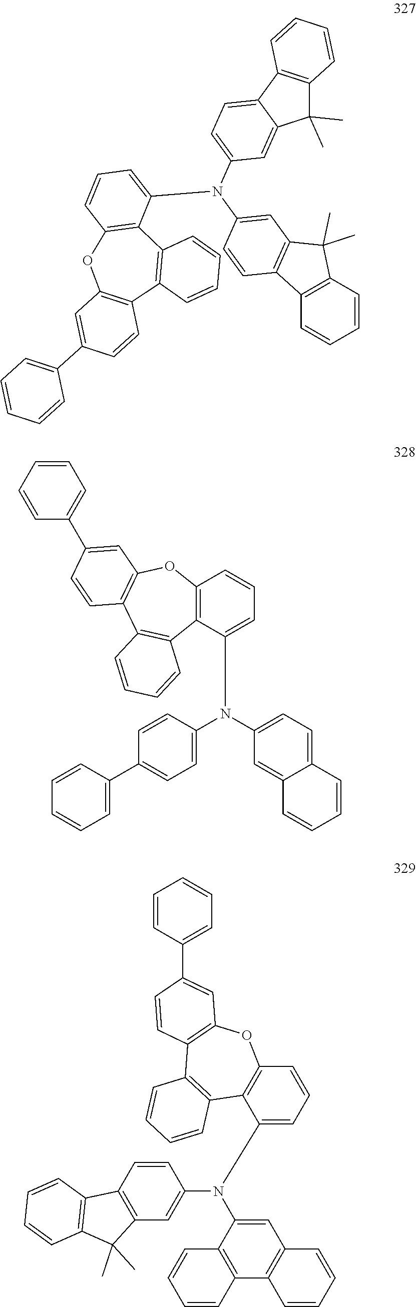

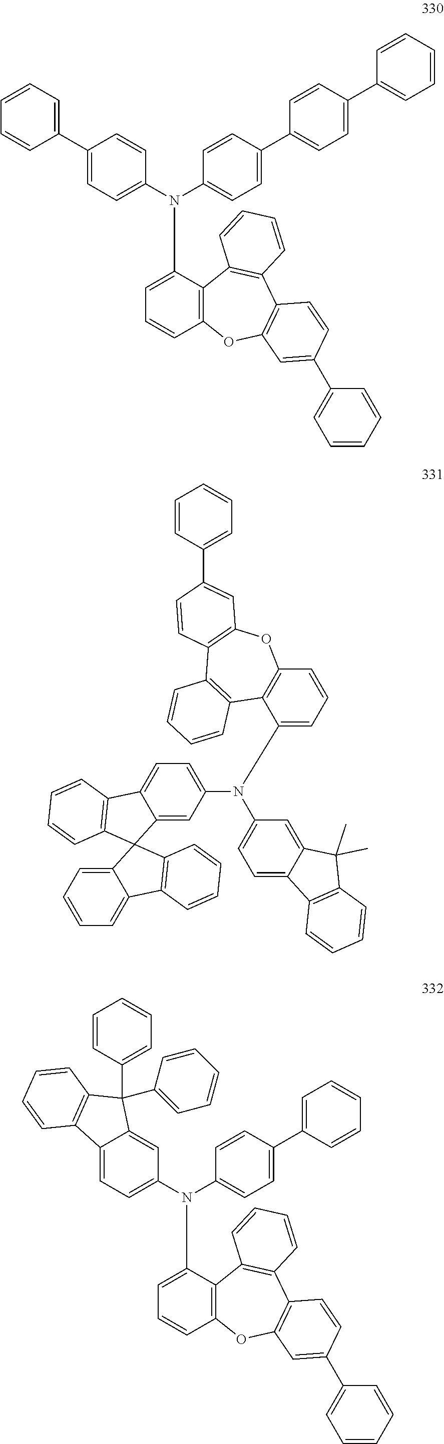 [SMILES: c1ccc(-c2ccc(-c3ccc(N(c4ccc(-c5ccccc5)cc4)c4cccc5c4-c4ccccc4-c4ccc(-c6ccccc6)cc4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3ccccc3-c3ccc(-c5ccccc5)cc3O4)cc21 ; c1ccc(-c2ccc(N(c3ccc4c(c3)C(c3ccccc3)(c3ccccc3)c3ccccc3-4)c3cccc4c3-c3ccccc3-c3ccc(-c5ccccc5)cc3O4)cc2)cc1]

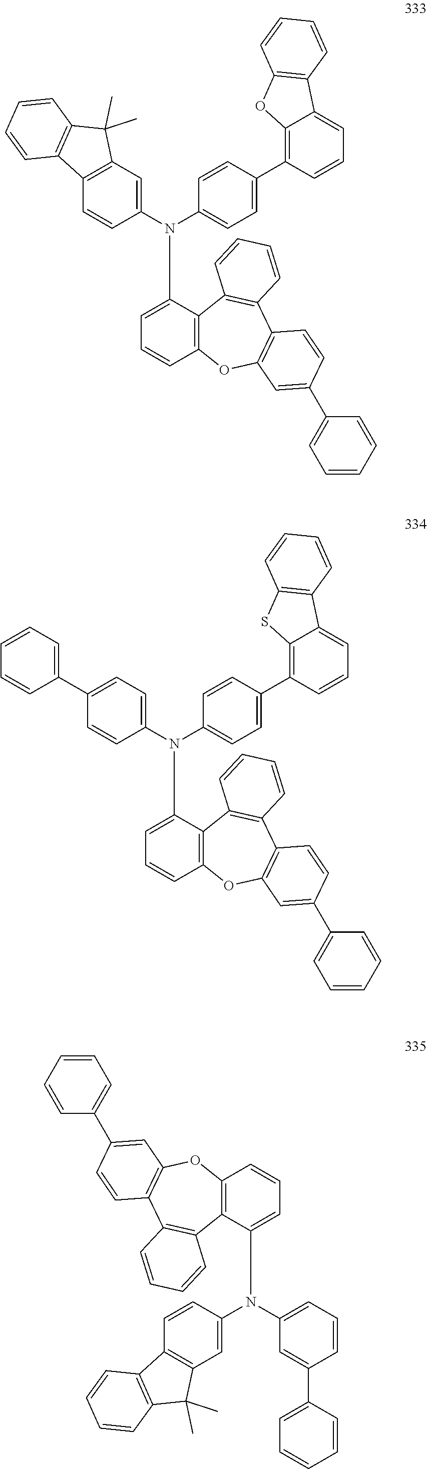 [SMILES: CC1(C)c2ccccc2-c2ccc(N(c3ccc(-c4cccc5c4oc4ccccc45)cc3)c3cccc4c3-c3ccccc3-c3ccc(-c5ccccc5)cc3O4)cc21 ; c1ccc(-c2ccc(N(c3ccc(-c4cccc5c4sc4ccccc45)cc3)c3cccc4c3-c3ccccc3-c3ccc(-c5ccccc5)cc3O4)cc2)cc1 ; CC1(C)c2ccccc2-c2ccc(N(c3cccc(-c4ccccc4)c3)c3cccc4c3-c3ccccc3-c3ccc(-c5ccccc5)cc3O4)cc21]

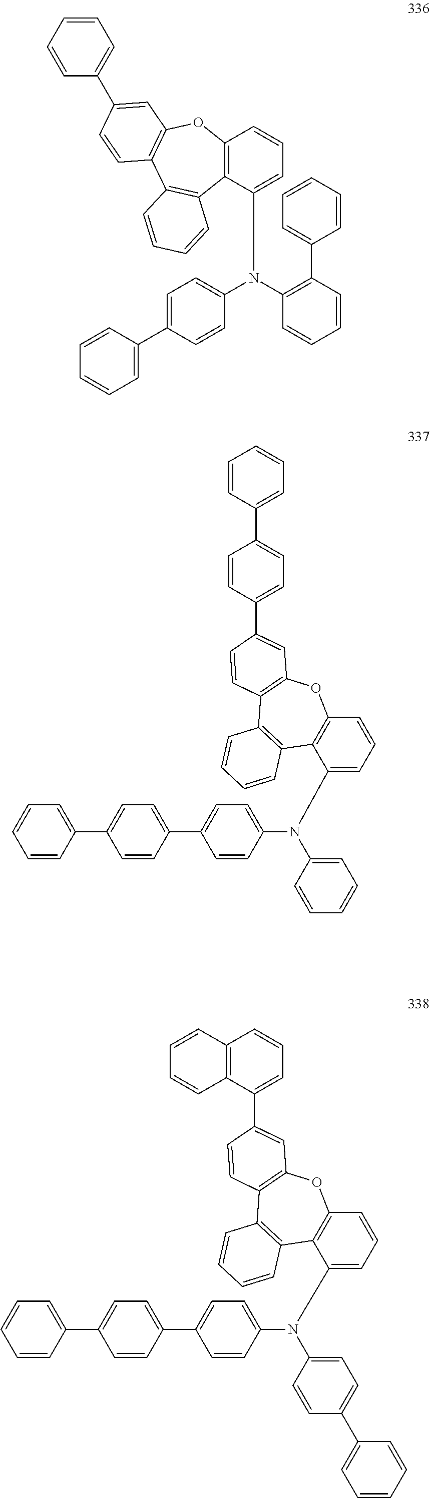 [SMILES: c1ccc(-c2ccc(N(c3ccccc3-c3ccccc3)c3cccc4c3-c3ccccc3-c3ccc(-c5ccccc5)cc3O4)cc2)cc1 ; c1ccc(-c2ccc(-c3ccc(N(c4ccccc4)c4cccc5c4-c4ccccc4-c4ccc(-c6ccc(-c7ccccc7)cc6)cc4O5)cc3)cc2)cc1 ; c1ccc(-c2ccc(-c3ccc(N(c4ccc(-c5ccccc5)cc4)c4cccc5c4-c4ccccc4-c4ccc(-c6cccc7ccccc67)cc4O5)cc3)cc2)cc1]

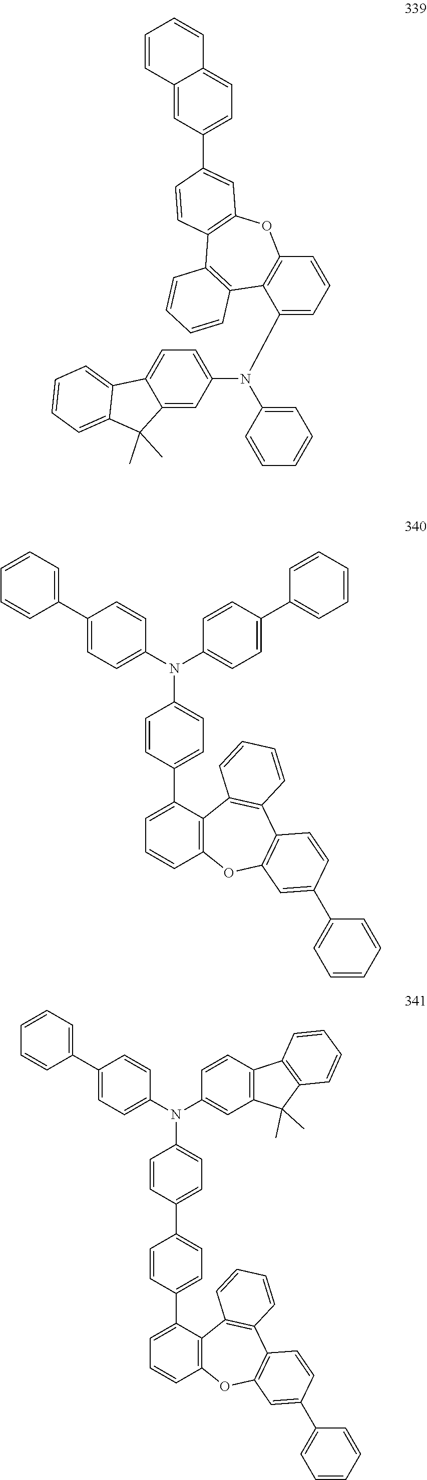 [SMILES: CC1(C)c2ccccc2-c2ccc(N(c3ccccc3)c3cccc4c3-c3ccccc3-c3ccc(-c5ccc6ccccc6c5)cc3O4)cc21 ; c1ccc(-c2ccc(N(c3ccc(-c4ccccc4)cc3)c3ccc(-c4cccc5c4-c4ccccc4-c4ccc(-c6ccccc6)cc4O5)cc3)cc2)cc1 ; CC1(C)c2ccccc2-c2ccc(N(c3ccc(-c4ccccc4)cc3)c3ccc(-c4ccc(-c5cccc6c5-c5ccccc5-c5ccc(-c7ccccc7)cc5O6)cc4)cc3)cc21]

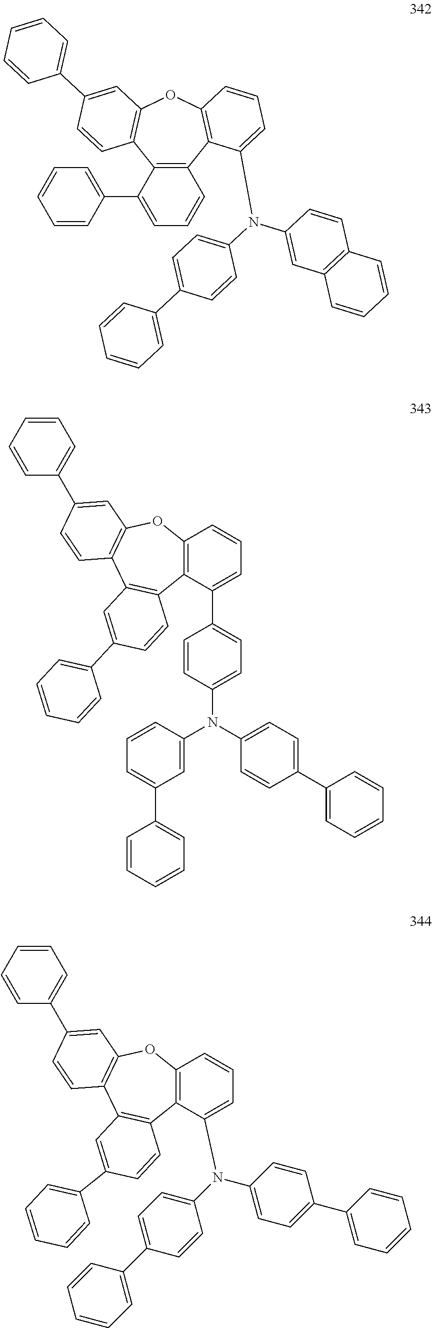 [SMILES: c1ccc(-c2ccc(N(c3ccc4ccccc4c3)c3cccc4c3-c3cccc(-c5ccccc5)c3-c3ccc(-c5ccccc5)cc3O4)cc2)cc1 ; c1ccc(-c2ccc(N(c3ccc(-c4cccc5c4-c4ccc(-c6ccccc6)cc4-c4ccc(-c6ccccc6)cc4O5)cc3)c3cccc(-c4ccccc4)c3)cc2)cc1 ; c1ccc(-c2ccc(N(c3ccc(-c4ccccc4)cc3)c3cccc4c3-c3ccc(-c5ccccc5)cc3-c3ccc(-c5ccccc5)cc3O4)cc2)cc1]

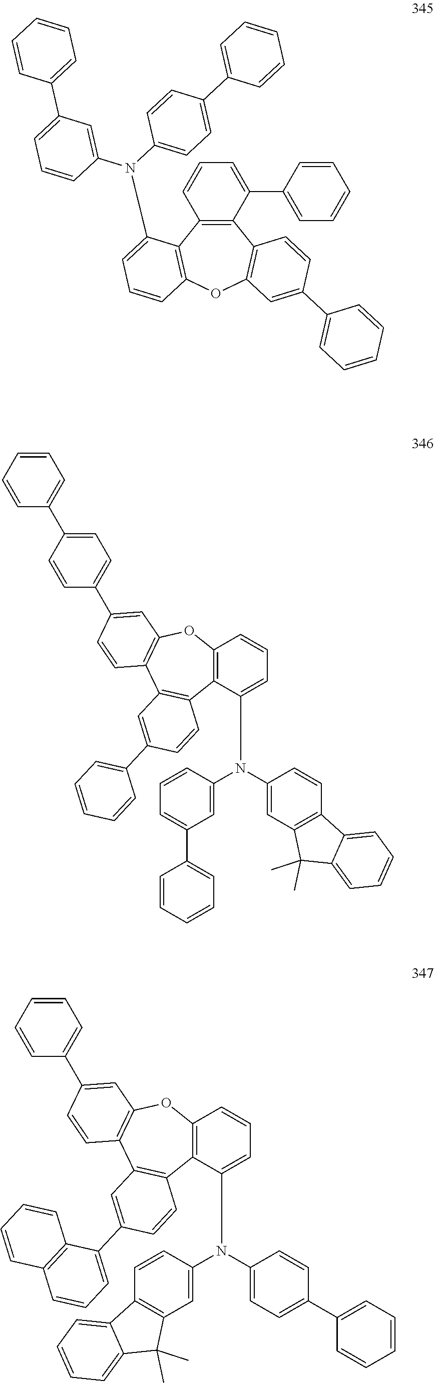 [SMILES: c1ccc(-c2ccc(N(c3cccc(-c4ccccc4)c3)c3cccc4c3-c3cccc(-c5ccccc5)c3-c3ccc(-c5ccccc5)cc3O4)cc2)cc1 ; CC1(C)c2ccccc2-c2ccc(N(c3cccc(-c4ccccc4)c3)c3cccc4c3-c3ccc(-c5ccccc5)cc3-c3ccc(-c5ccc(-c6ccccc6)cc5)cc3O4)cc21 ; CC1(C)c2ccccc2-c2ccc(N(c3ccc(-c4ccccc4)cc3)c3cccc4c3-c3ccc(-c5cccc6ccccc56)cc3-c3ccc(-c5ccccc5)cc3O4)cc21]

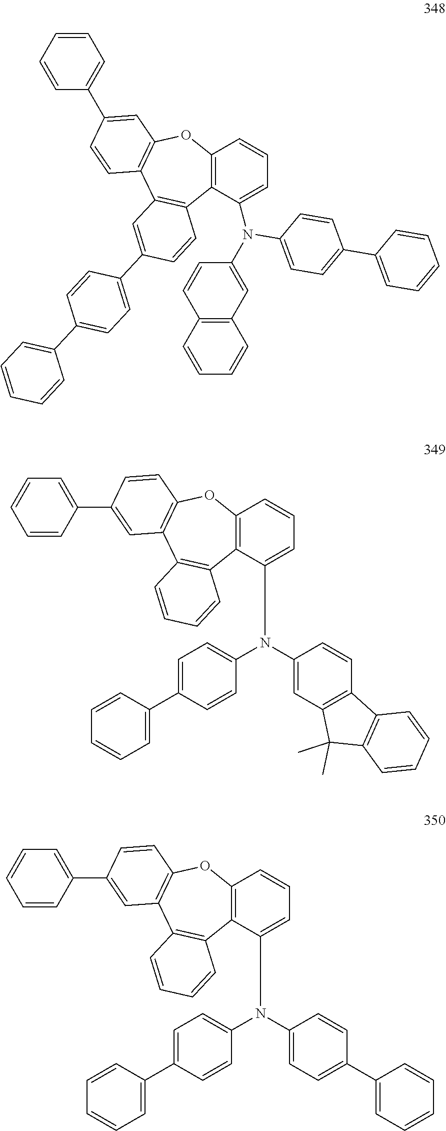 [SMILES: c1ccc(-c2ccc(-c3ccc4c(c3)-c3ccc(-c5ccccc5)cc3Oc3cccc(N(c5ccc(-c6ccccc6)cc5)c5ccc6ccccc6c5)c3-4)cc2)cc1 ; CC1(C)c2ccccc2-c2ccc(N(c3ccc(-c4ccccc4)cc3)c3cccc4c3-c3ccccc3-c3cc(-c5ccccc5)ccc3O4)cc21 ; c1ccc(-c2ccc(N(c3ccc(-c4ccccc4)cc3)c3cccc4c3-c3ccccc3-c3cc(-c5ccccc5)ccc3O4)cc2)cc1]

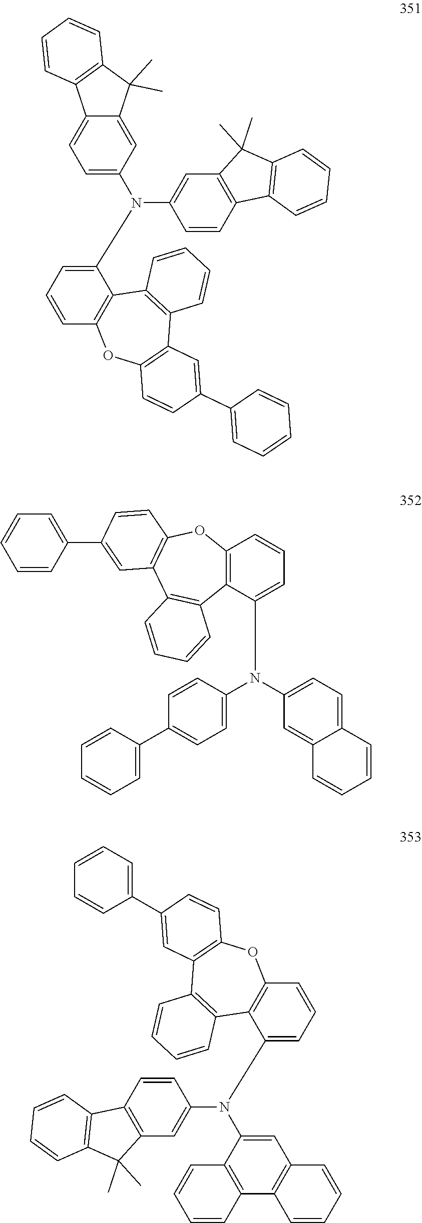 [SMILES: CC1(C)c2ccccc2-c2ccc(N(c3ccc4c(c3)C(C)(C)c3ccccc3-4)c3cccc4c3-c3ccccc3-c3cc(-c5ccccc5)ccc3O4)cc21 ; c1ccc(-c2ccc(N(c3ccc4ccccc4c3)c3cccc4c3-c3ccccc3-c3cc(-c5ccccc5)ccc3O4)cc2)cc1 ; CC1(C)c2ccccc2-c2ccc(N(c3cccc4c3-c3ccccc3-c3cc(-c5ccccc5)ccc3O4)c3cc4ccccc4c4ccccc34)cc21]

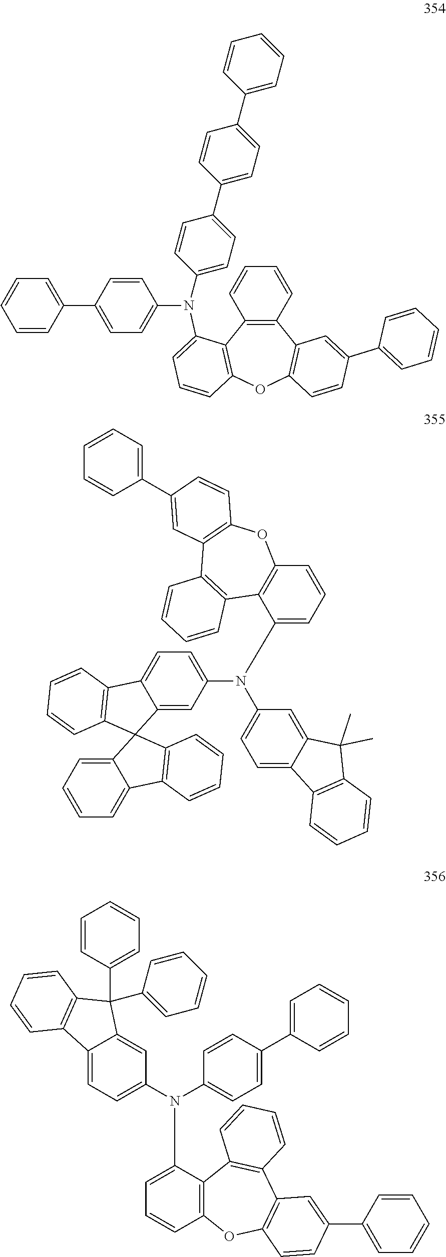 [SMILES: c1ccc(-c2ccc(-c3ccc(N(c4ccc(-c5ccccc5)cc4)c4cccc5c4-c4ccccc4-c4cc(-c6ccccc6)ccc4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3ccccc3-c3cc(-c5ccccc5)ccc3O4)cc21 ; c1ccc(-c2ccc(N(c3ccc4c(c3)C(c3ccccc3)(c3ccccc3)c3ccccc3-4)c3cccc4c3-c3ccccc3-c3cc(-c5ccccc5)ccc3O4)cc2)cc1]

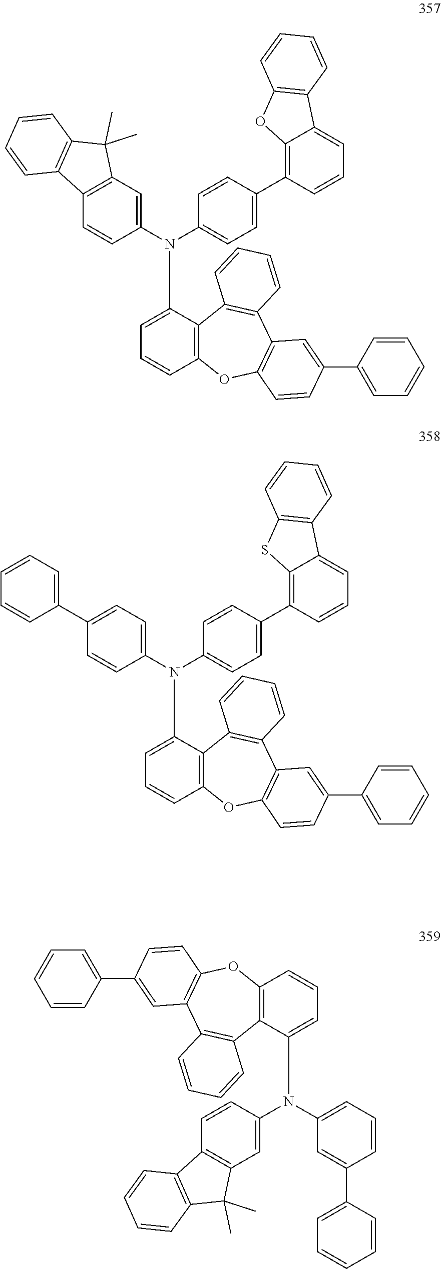 [SMILES: CC1(C)c2ccccc2-c2ccc(N(c3ccc(-c4cccc5c4oc4ccccc45)cc3)c3cccc4c3-c3ccccc3-c3cc(-c5ccccc5)ccc3O4)cc21 ; c1ccc(-c2ccc(N(c3ccc(-c4cccc5c4sc4ccccc45)cc3)c3cccc4c3-c3ccccc3-c3cc(-c5ccccc5)ccc3O4)cc2)cc1 ; CC1(C)c2ccccc2-c2ccc(N(c3cccc(-c4ccccc4)c3)c3cccc4c3-c3ccccc3-c3cc(-c5ccccc5)ccc3O4)cc21]

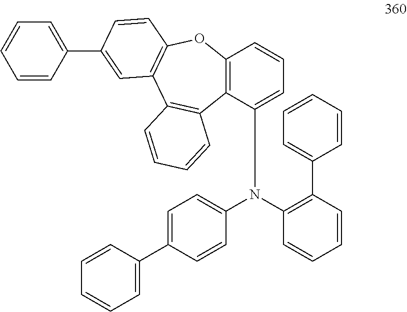 [SMILES: c1ccc(-c2ccc(N(c3ccccc3-c3ccccc3)c3cccc4c3-c3ccccc3-c3cc(-c5ccccc5)ccc3O4)cc2)cc1]

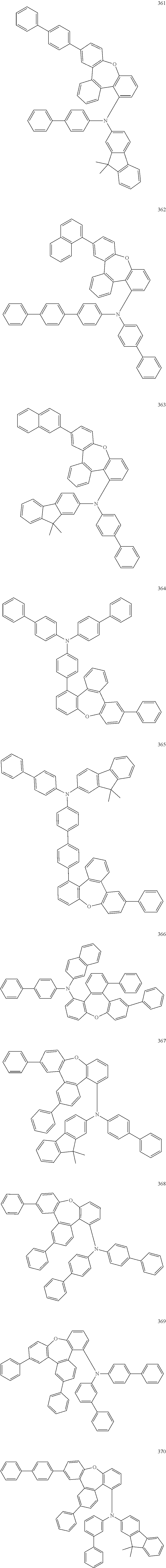

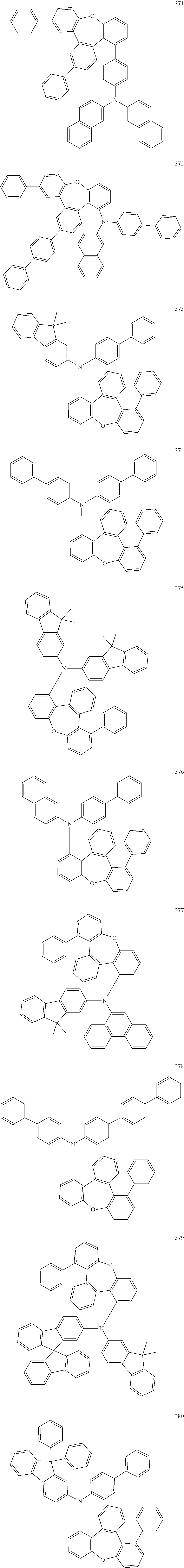

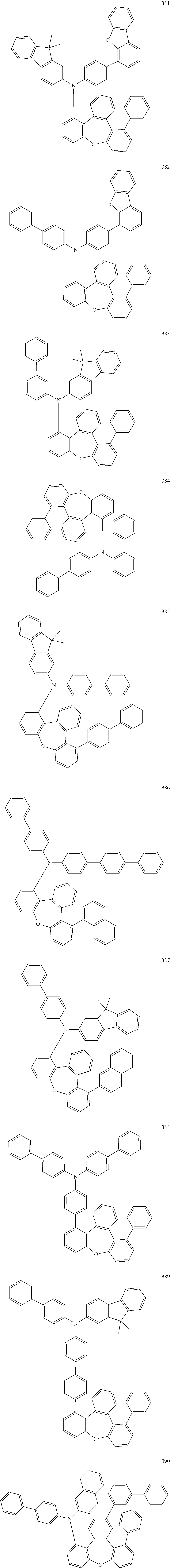

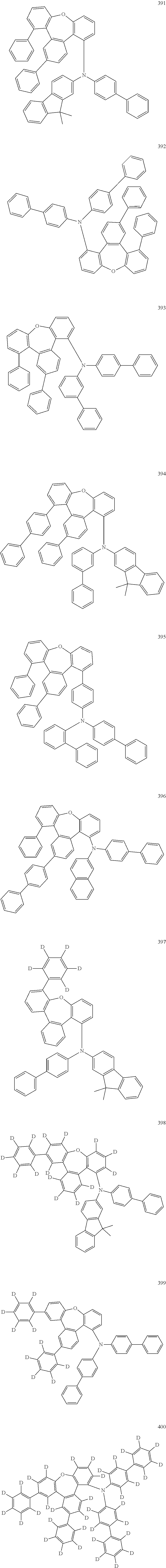

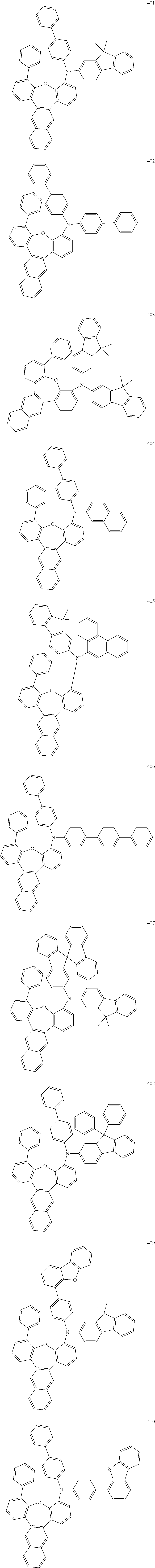

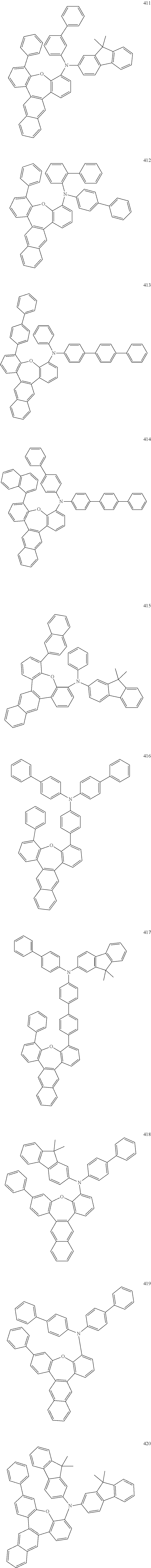

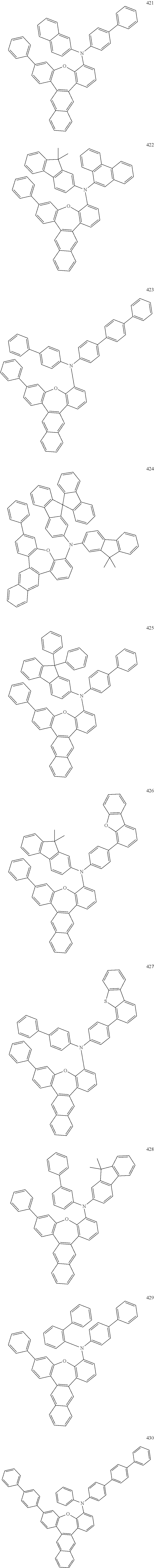

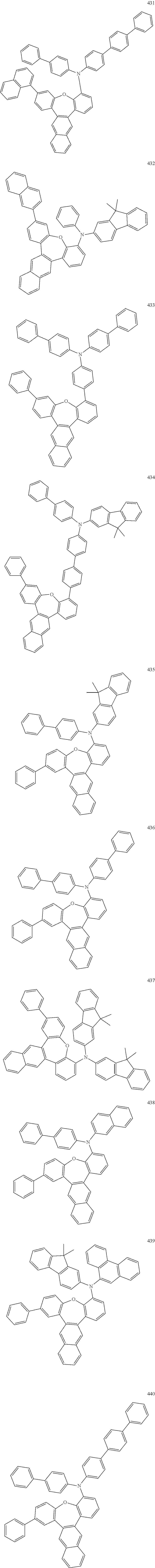

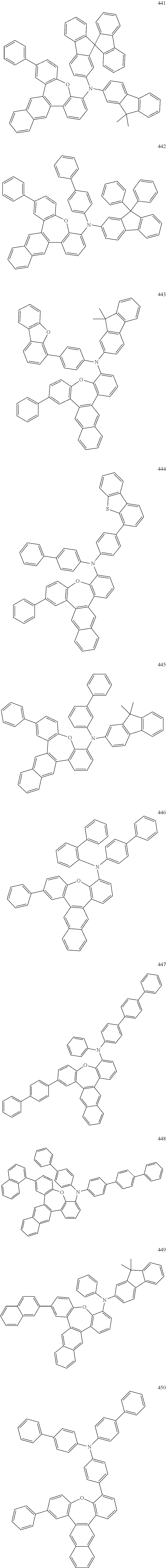

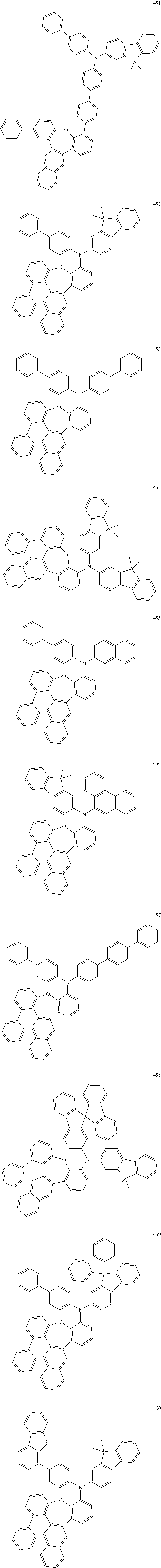

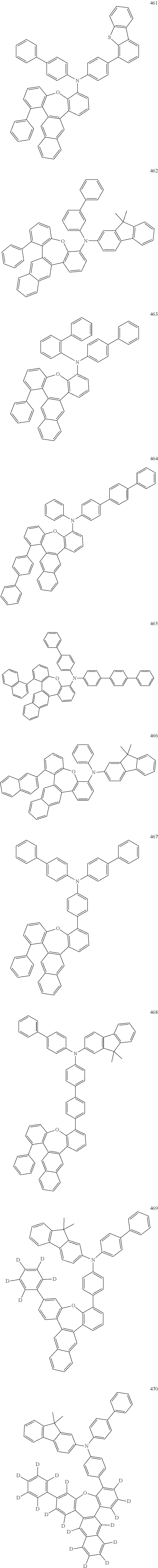

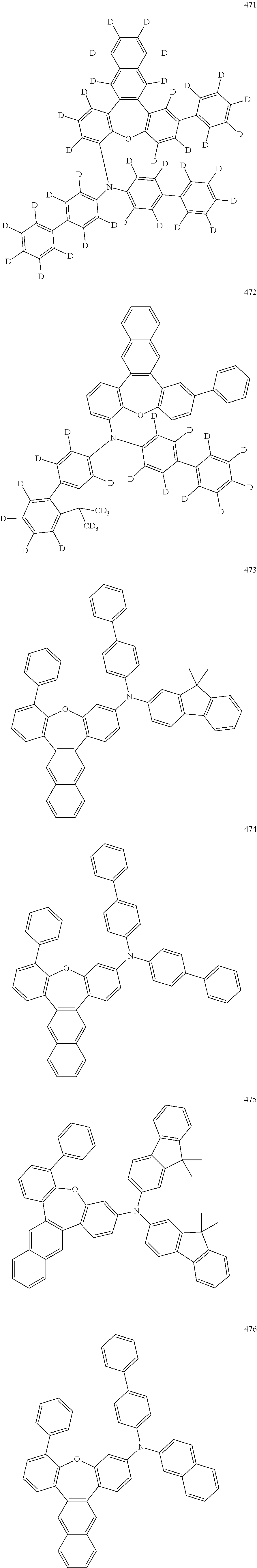

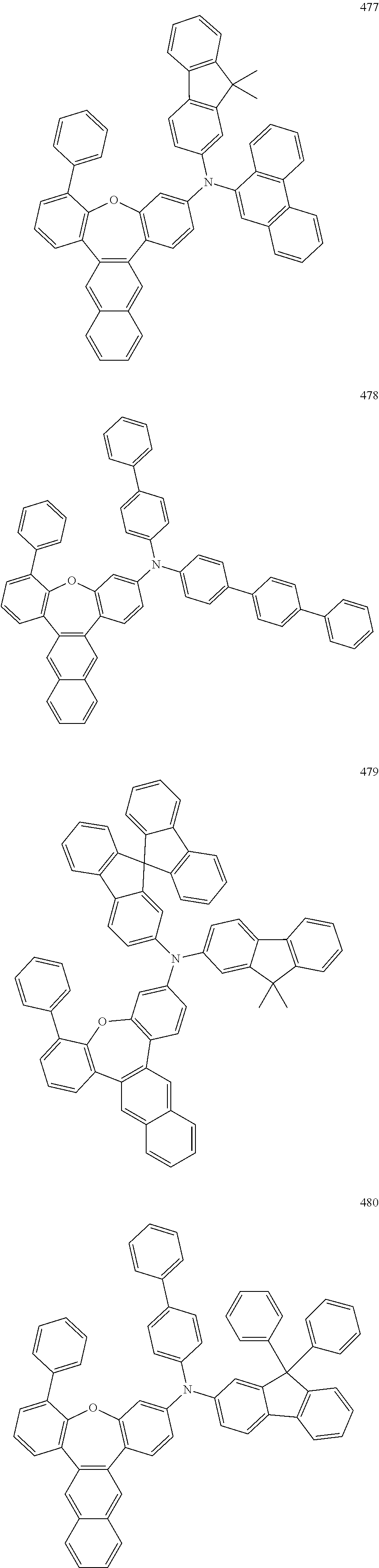 [SMILES: CC1(C)c2ccccc2-c2ccc(N(c3ccc4c(c3)Oc3c(-c5ccccc5)cccc3-c3cc5ccccc5cc3-4)c3cc4ccccc4c4ccccc34)cc21 ; c1ccc(-c2ccc(-c3ccc(N(c4ccc(-c5ccccc5)cc4)c4ccc5c(c4)Oc4c(-c6ccccc6)cccc4-c4cc6ccccc6cc4-5)cc3)cc2)cc1 ; CC1(C)c2ccccc2-c2ccc(N(c3ccc4c(c3)Oc3c(-c5ccccc5)cccc3-c3cc5ccccc5cc3-4)c3ccc4c(c3)C3(c5ccccc5-c5ccccc53)c3ccccc3-4)cc21 ; c1ccc(-c2ccc(N(c3ccc4c(c3)Oc3c(-c5ccccc5)cccc3-c3cc5ccccc5cc3-4)c3ccc4c(c3)C(c3ccccc3)(c3ccccc3)c3ccccc3-4)cc2)cc1]

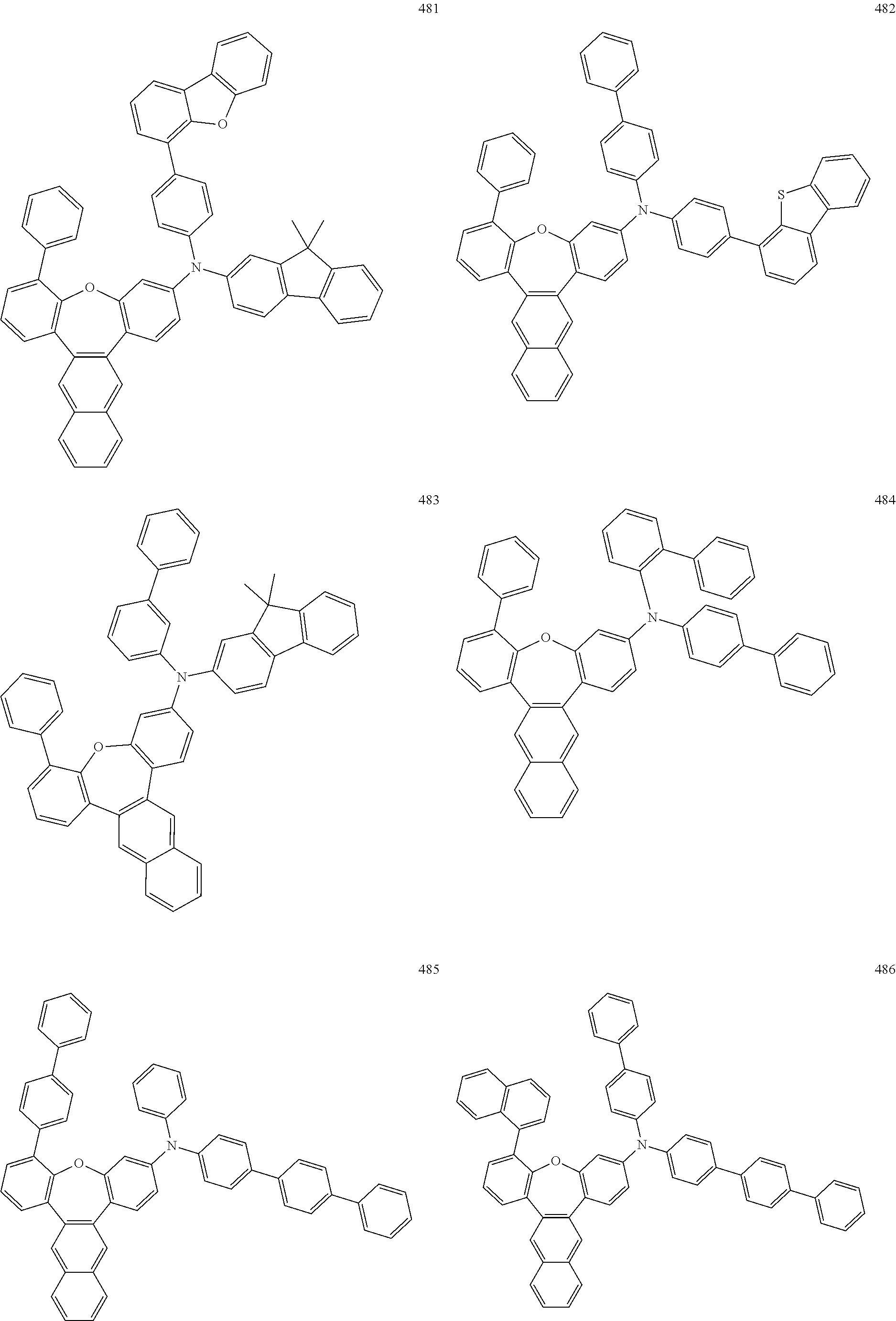

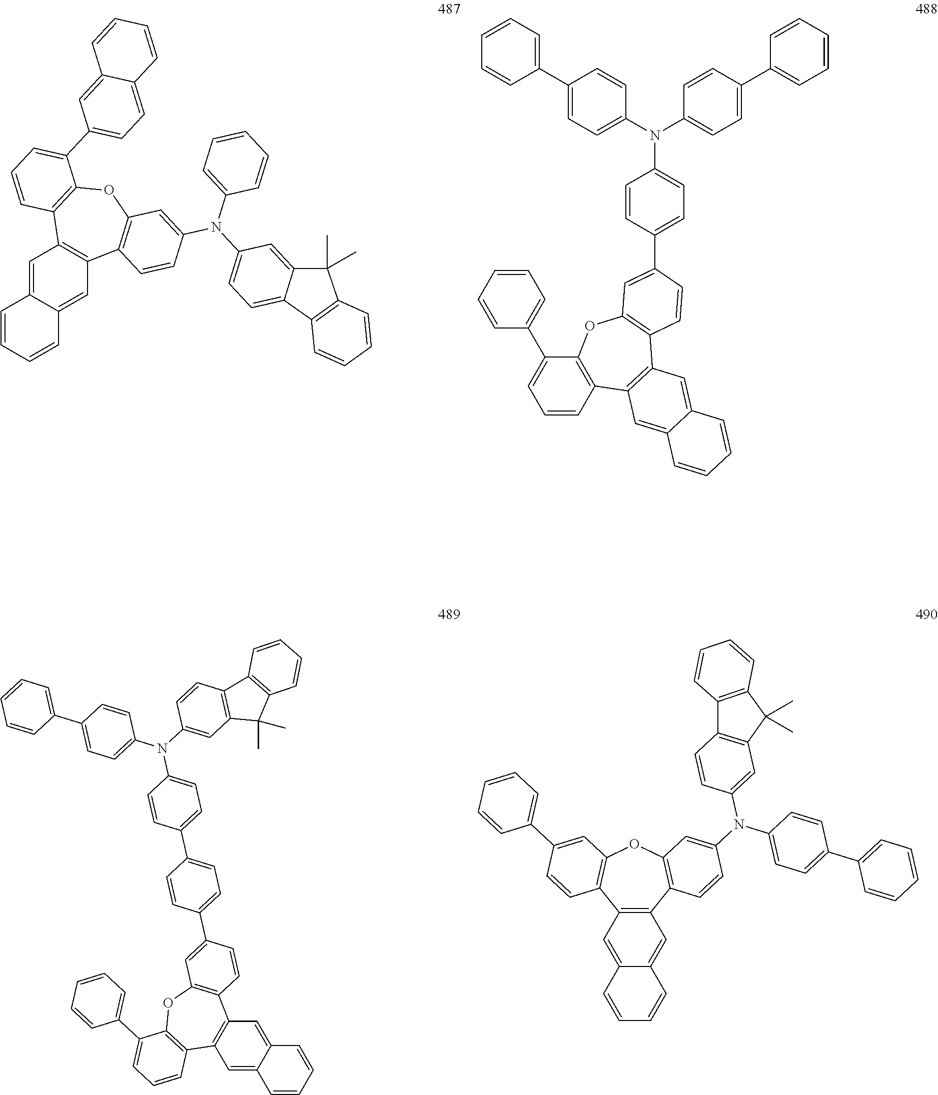 [SMILES: CC1(C)c2ccccc2-c2ccc(N(c3ccccc3)c3ccc4c(c3)Oc3c(-c5ccc6ccccc6c5)cccc3-c3cc5ccccc5cc3-4)cc21 ; c1ccc(-c2ccc(N(c3ccc(-c4ccccc4)cc3)c3ccc(-c4ccc5c(c4)Oc4c(-c6ccccc6)cccc4-c4cc6ccccc6cc4-5)cc3)cc2)cc1 ; CC1(C)c2ccccc2-c2ccc(N(c3ccc(-c4ccccc4)cc3)c3ccc(-c4ccc(-c5ccc6c(c5)Oc5c(-c7ccccc7)cccc5-c5cc7ccccc7cc5-6)cc4)cc3)cc21 ; CC1(C)c2ccccc2-c2ccc(N(c3ccc(-c4ccccc4)cc3)c3ccc4c(c3)Oc3cc(-c5ccccc5)ccc3-c3cc5ccccc5cc3-4)cc21]

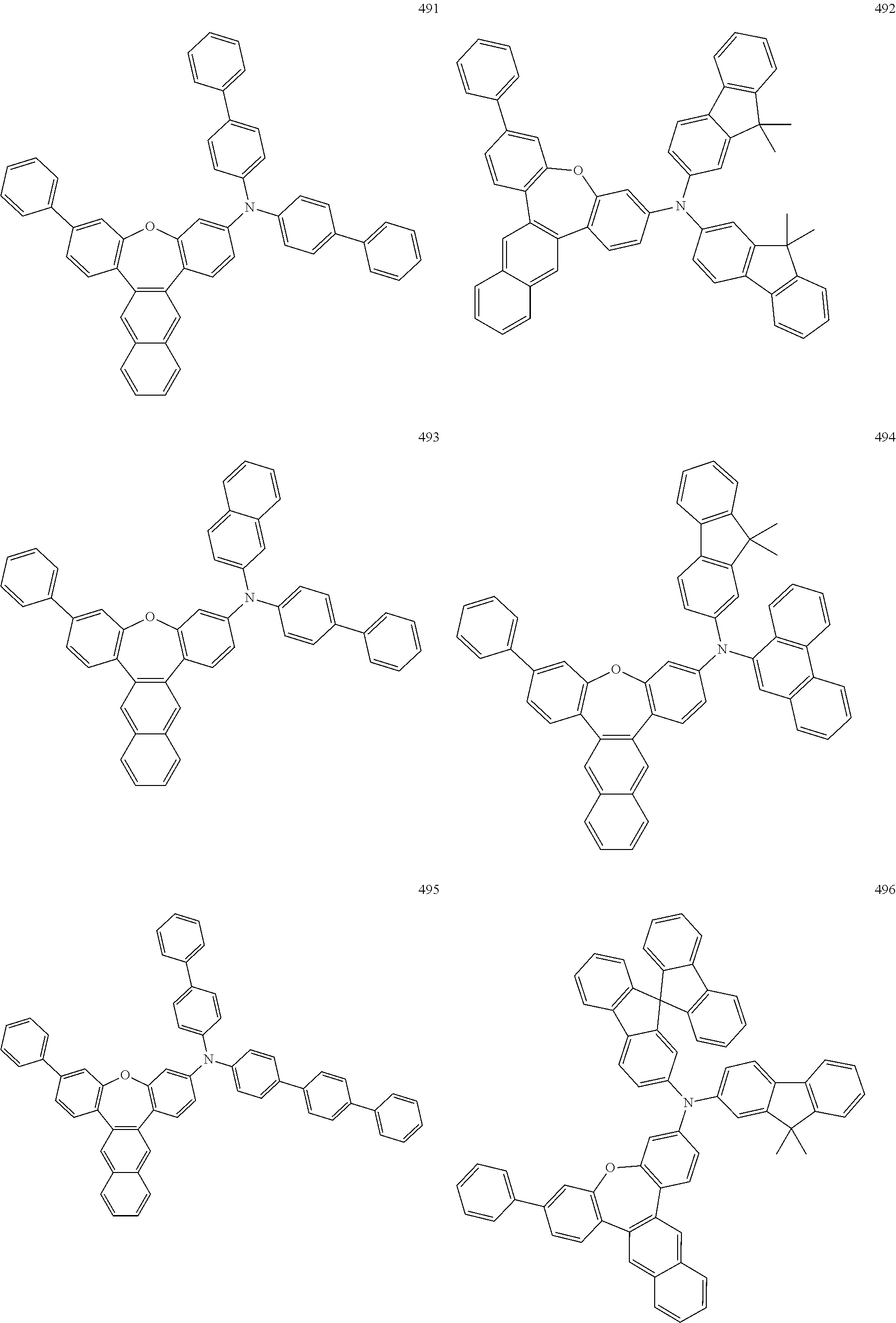

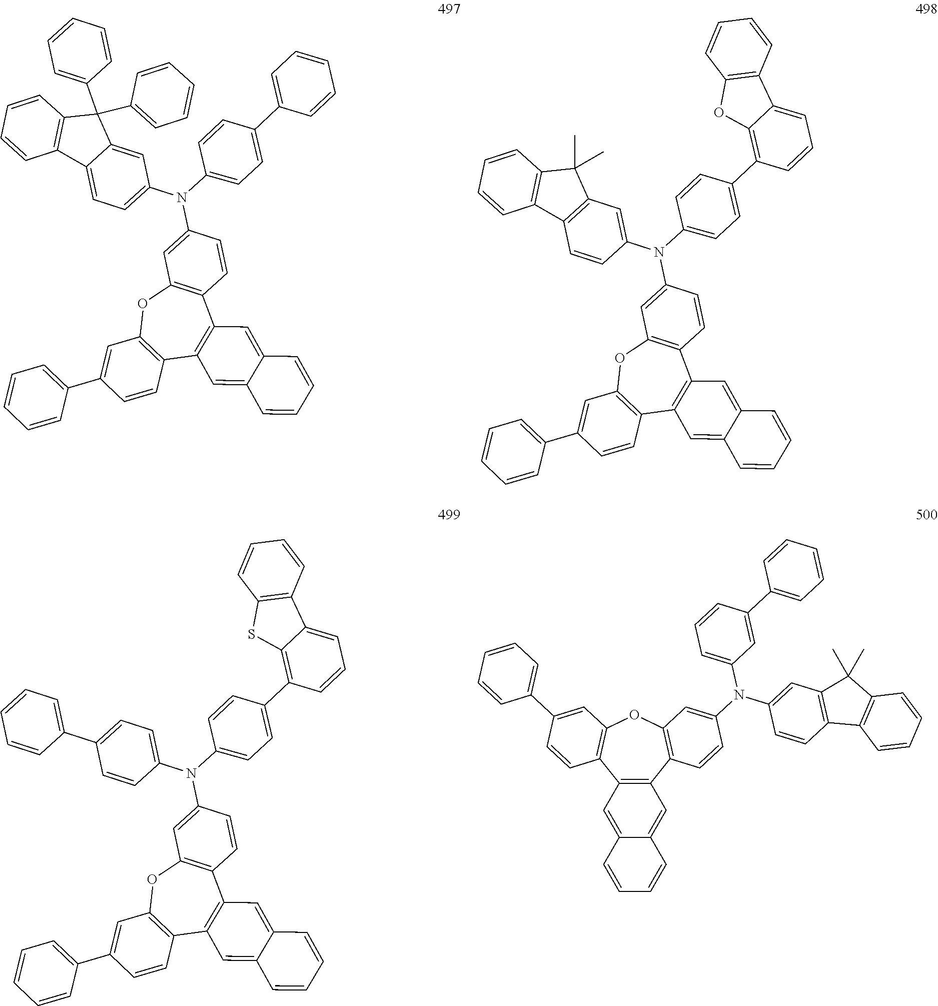 [SMILES: c1ccc(-c2ccc(N(c3ccc4c(c3)Oc3cc(-c5ccccc5)ccc3-c3cc5ccccc5cc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc(-c5ccccc5)ccc3-c3cc5ccccc5cc3-4)cc21 ; c1ccc(-c2ccc(N(c3ccc(-c4cccc5c4sc4ccccc45)cc3)c3ccc4c(c3)Oc3cc(-c5ccccc5)ccc3-c3cc5ccccc5cc3-4)cc2)cc1 ; CC1(C)c2ccccc2-c2ccc(N(c3cccc(-c4ccccc4)c3)c3ccc4c(c3)Oc3cc(-c5ccccc5)ccc3-c3cc5ccccc5cc3-4)cc21]

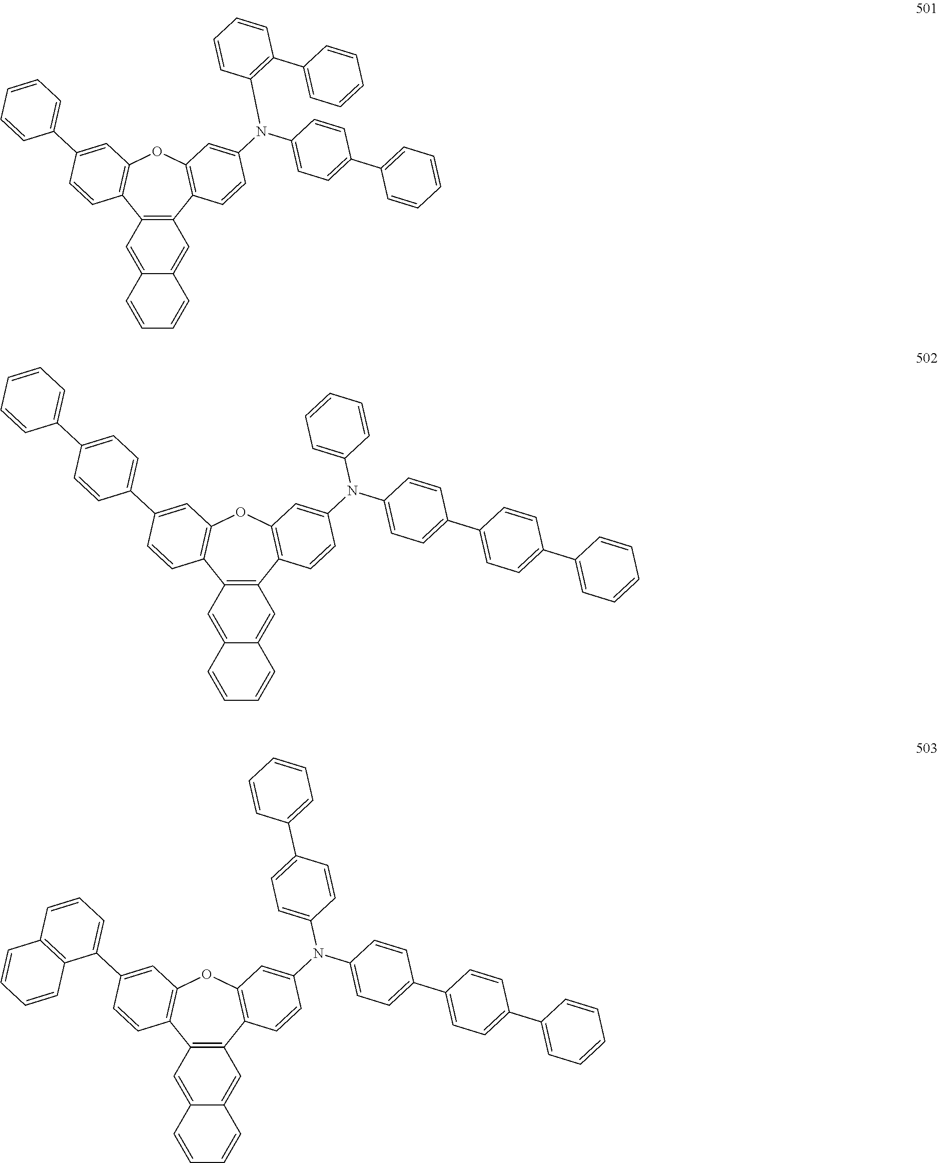 [SMILES: c1ccc(-c2ccc(N(c3ccc4c(c3)Oc3cc(-c5ccccc5)ccc3-c3cc5ccccc5cc3-4)c3ccccc3-c3ccccc3)cc2)cc1 ; c1ccc(-c2ccc(-c3ccc(N(c4ccccc4)c4ccc5c(c4)Oc4cc(-c6ccc(-c7ccccc7)cc6)ccc4-c4cc6ccccc6cc4-5)cc3)cc2)cc1 ; c1ccc(-c2ccc(-c3ccc(N(c4ccc(-c5ccccc5)cc4)c4ccc5c(c4)Oc4cc(-c6cccc7ccccc67)ccc4-c4cc6ccccc6cc4-5)cc3)cc2)cc1]

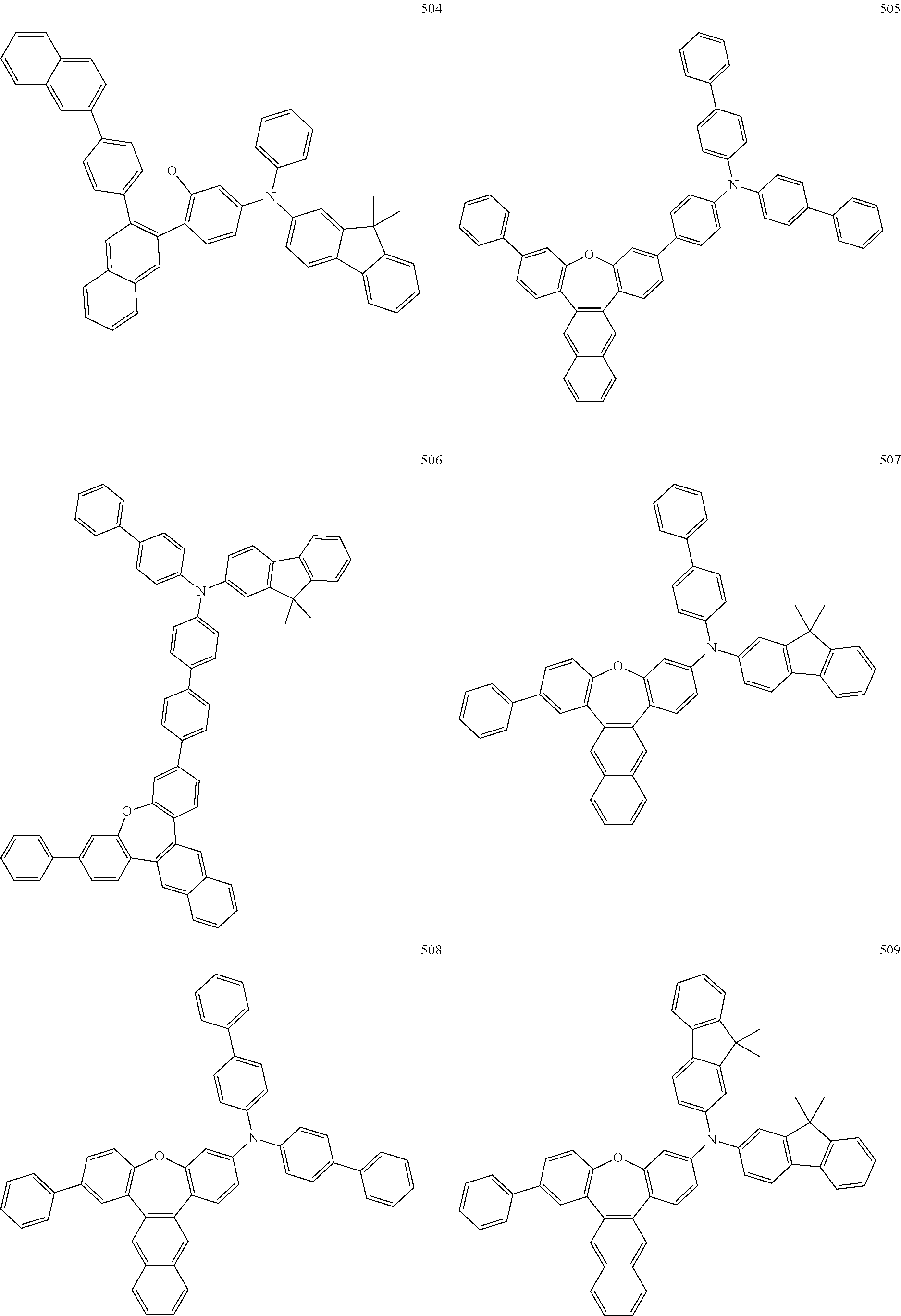

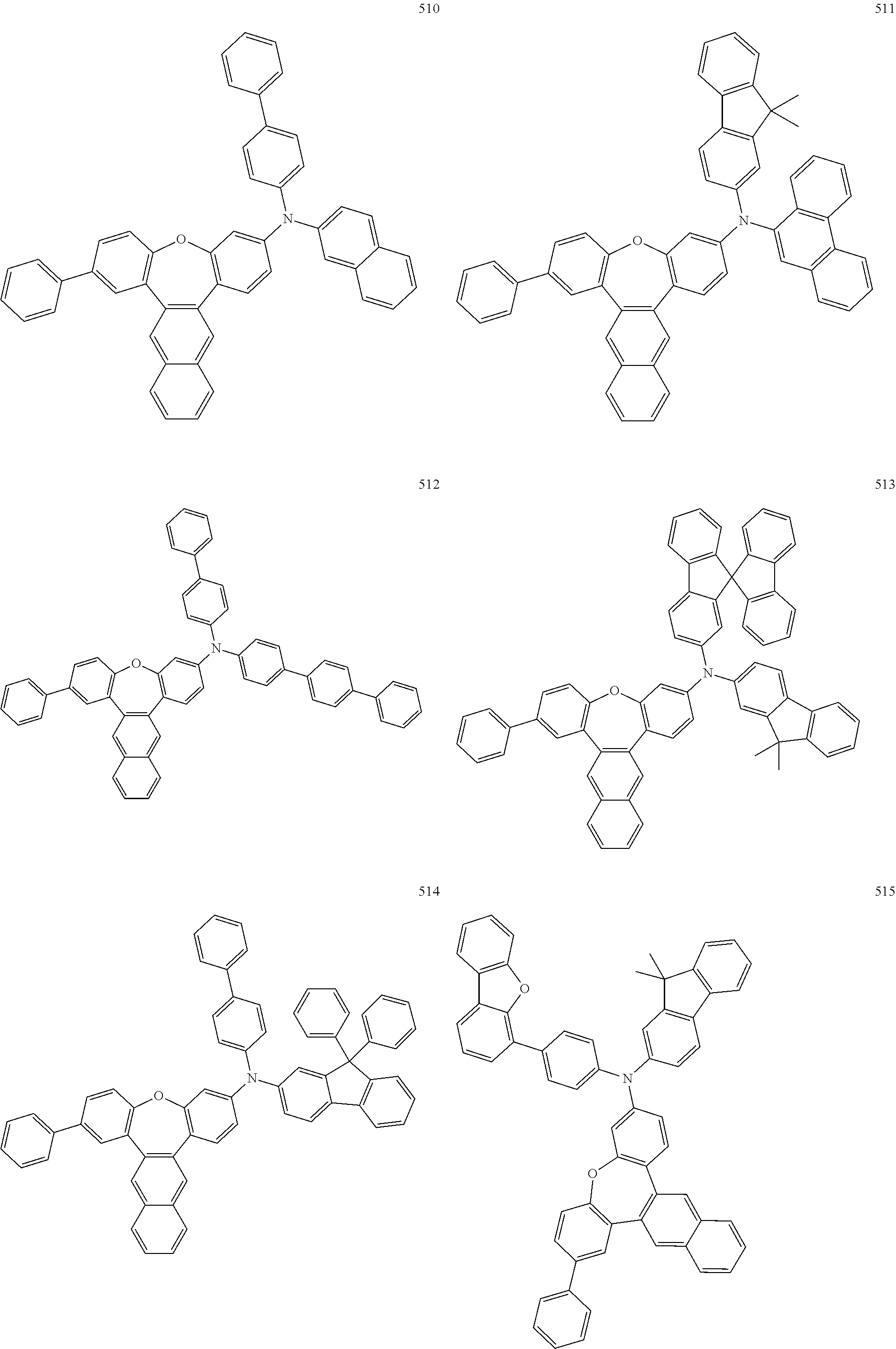

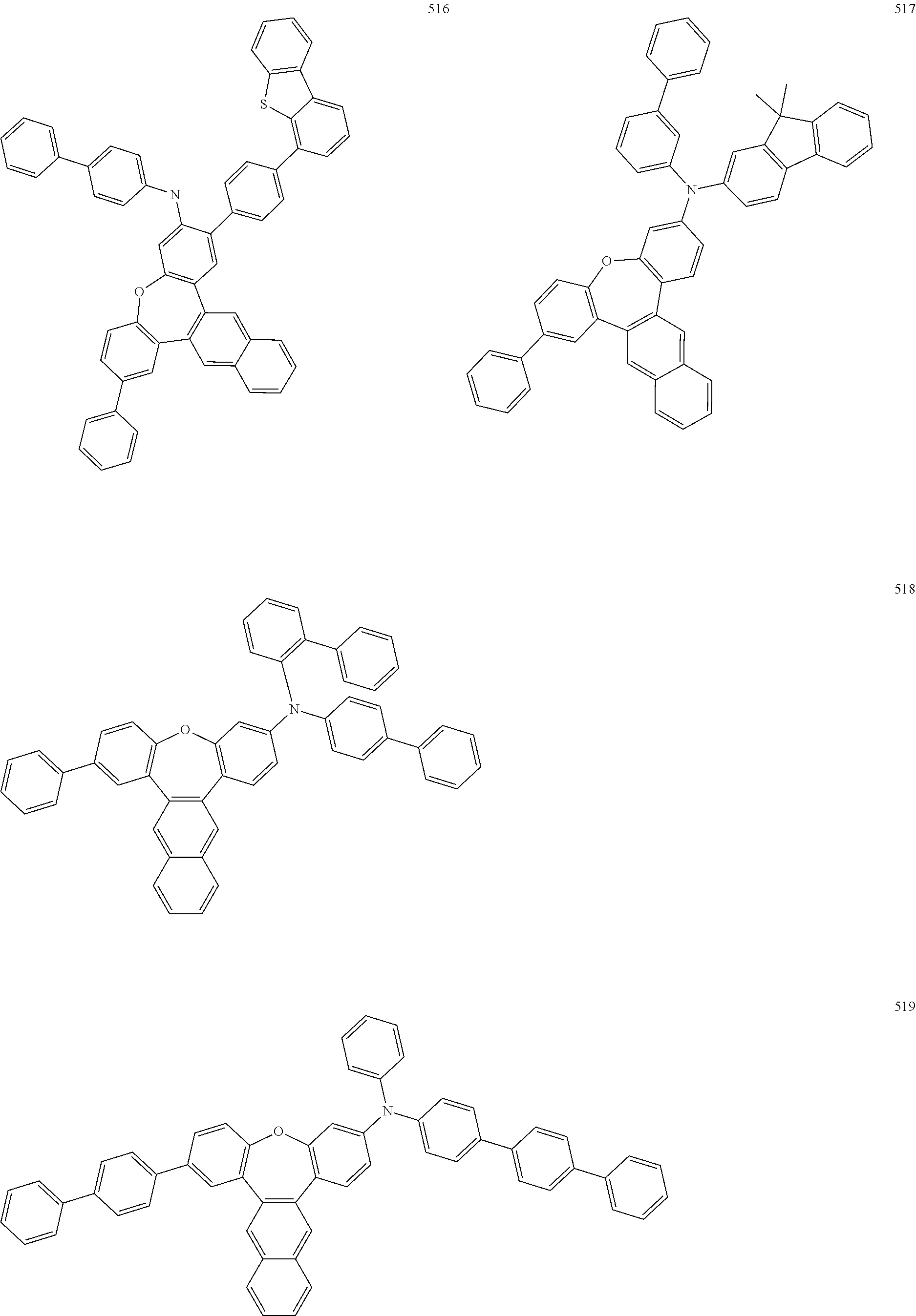 [SMILES: c1ccc(-c2ccc(Nc3cc4c(cc3-c3ccc(-c5cccc6c5sc5ccccc56)cc3)-c3cc5ccccc5cc3-c3cc(-c5ccccc5)ccc3O4)cc2)cc1 ; CC1(C)c2ccccc2-c2ccc(N(c3cccc(-c4ccccc4)c3)c3ccc4c(c3)Oc3ccc(-c5ccccc5)cc3-c3cc5ccccc5cc3-4)cc21 ; c1ccc(-c2ccc(N(c3ccc4c(c3)Oc3ccc(-c5ccccc5)cc3-c3cc5ccccc5cc3-4)c3ccccc3-c3ccccc3)cc2)cc1 ; c1ccc(-c2ccc(-c3ccc(N(c4ccccc4)c4ccc5c(c4)Oc4ccc(-c6ccc(-c7ccccc7)cc6)cc4-c4cc6ccccc6cc4-5)cc3)cc2)cc1]

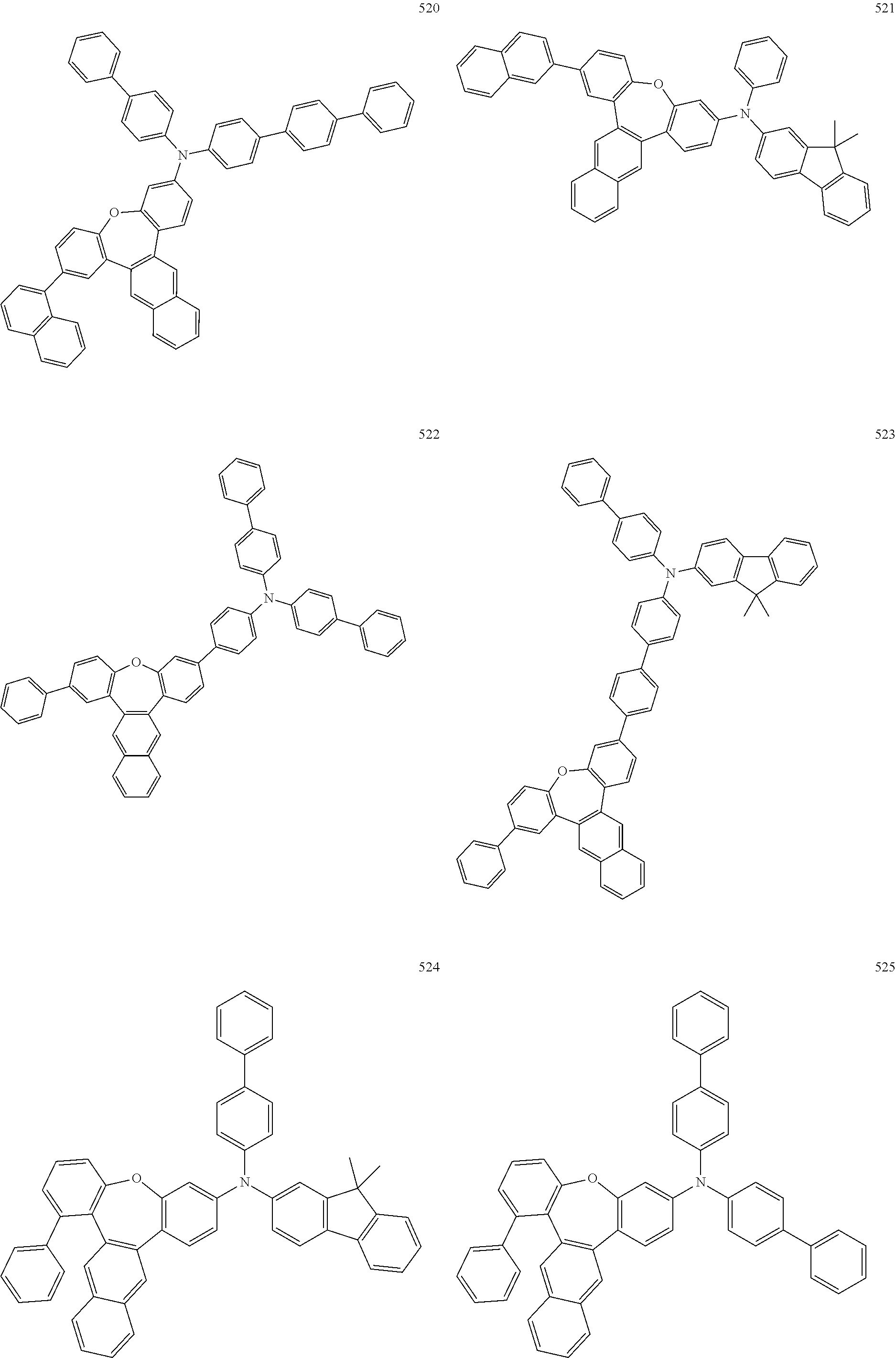

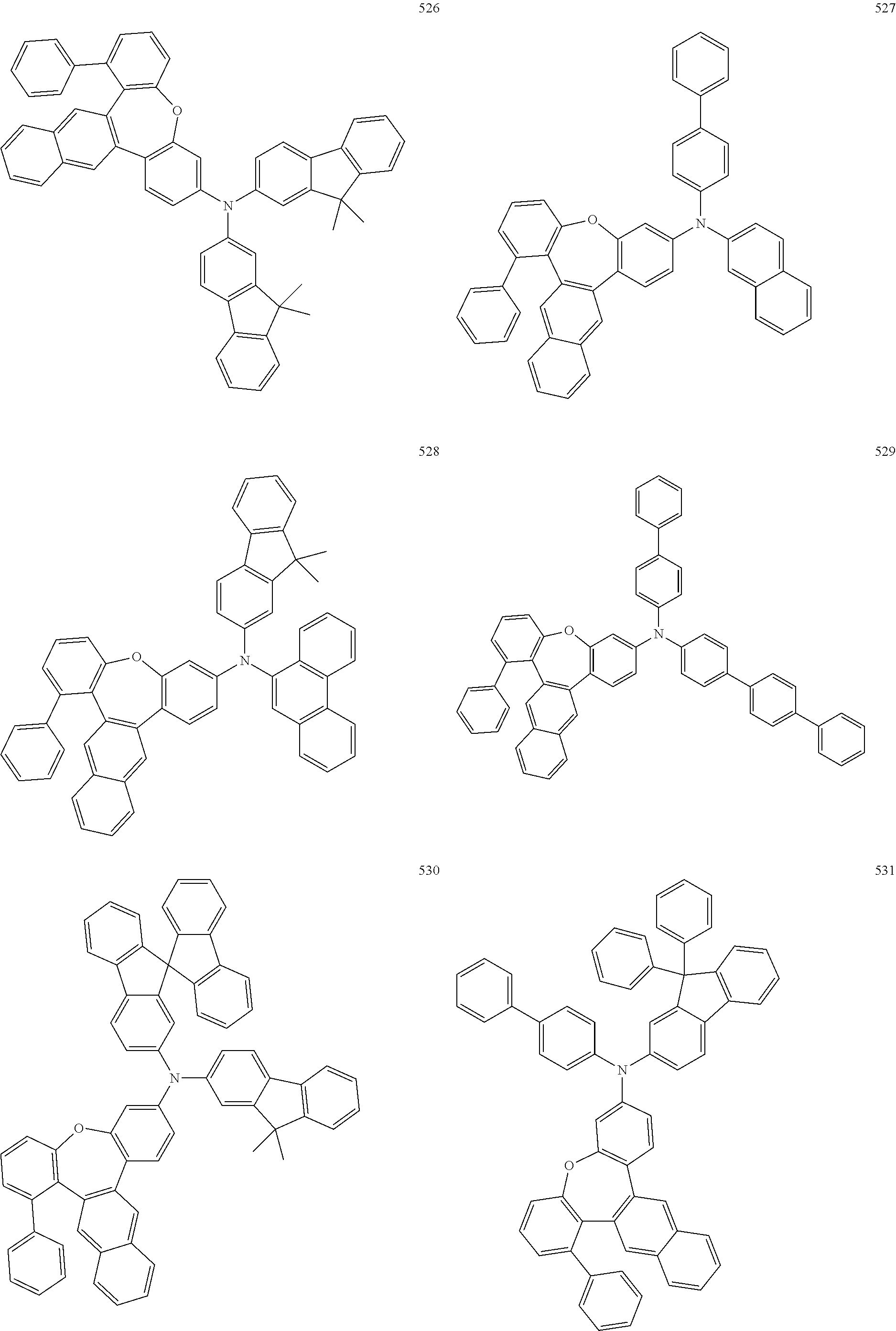

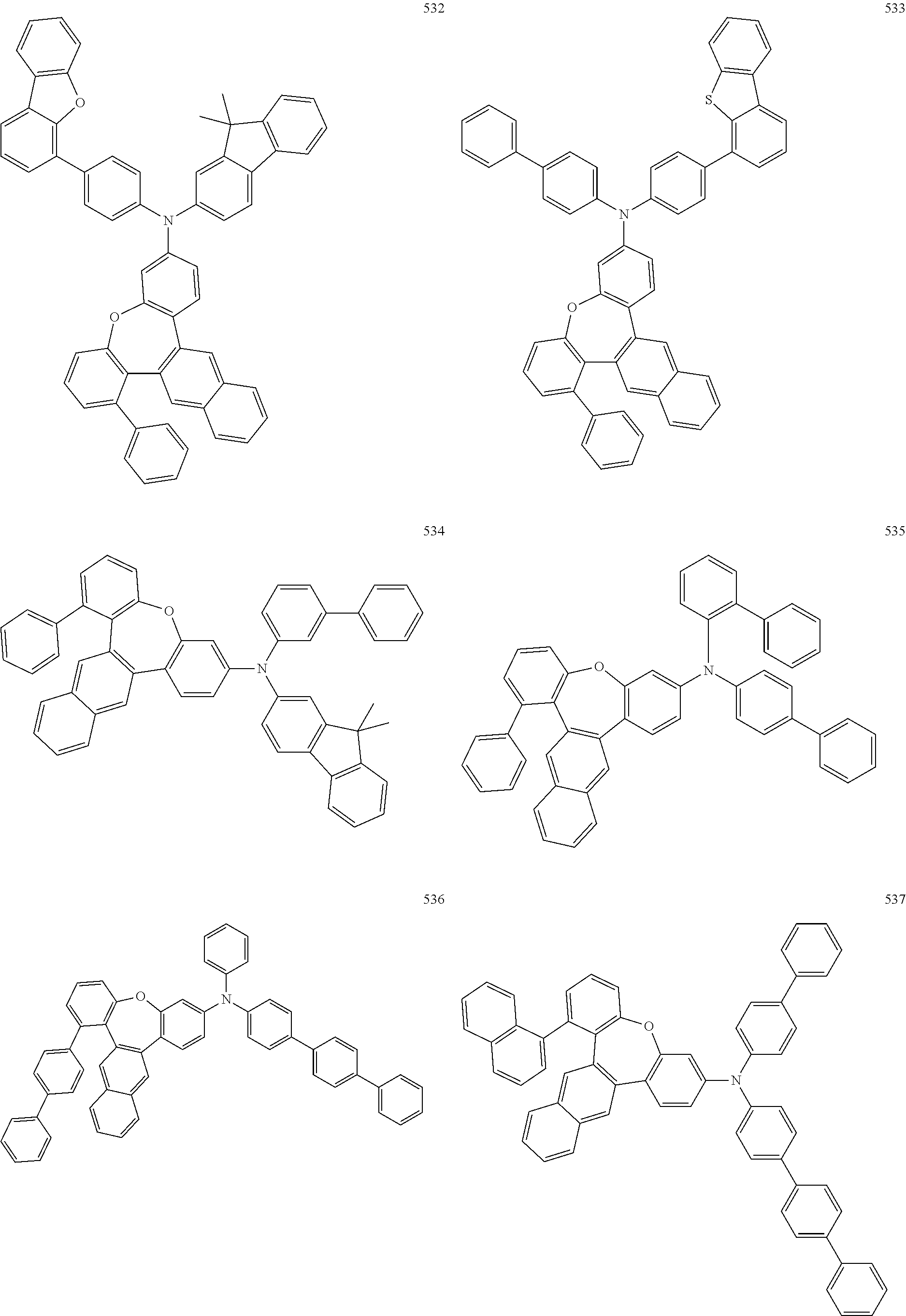

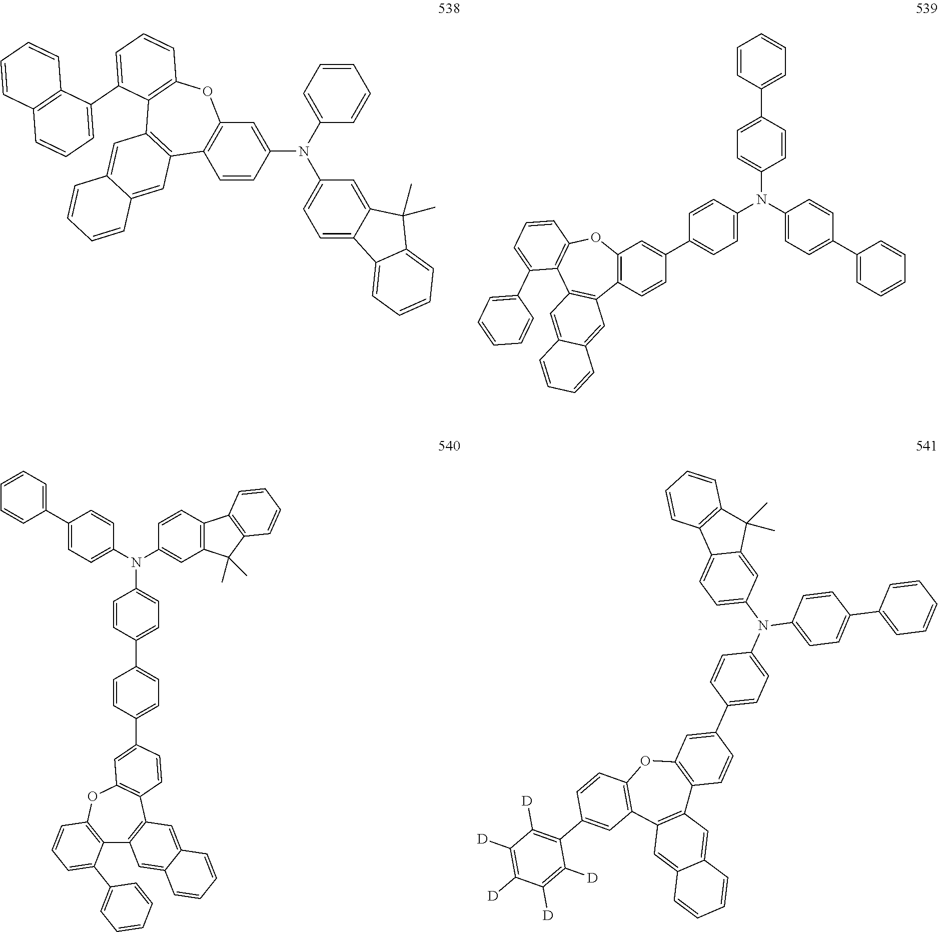 [SMILES: CC1(C)c2ccccc2-c2ccc(N(c3ccccc3)c3ccc4c(c3)Oc3cccc(-c5cccc6ccccc56)c3-c3cc5ccccc5cc3-4)cc21 ; c1ccc(-c2ccc(N(c3ccc(-c4ccccc4)cc3)c3ccc(-c4ccc5c(c4)Oc4cccc(-c6ccccc6)c4-c4cc6ccccc6cc4-5)cc3)cc2)cc1 ; CC1(C)c2ccccc2-c2ccc(N(c3ccc(-c4ccccc4)cc3)c3ccc(-c4ccc(-c5ccc6c(c5)Oc5cccc(-c7ccccc7)c5-c5cc7ccccc7cc5-6)cc4)cc3)cc21 ; [2H]c1c([2H])c([2H])c(-c2ccc3c(c2)-c2cc4ccccc4cc2-c2ccc(-c4ccc(N(c5ccc(-c6ccccc6)cc5)c5ccc6c(c5)C(C)(C)c5ccccc5-6)cc4)cc2O3)c([2H])c1[2H]]

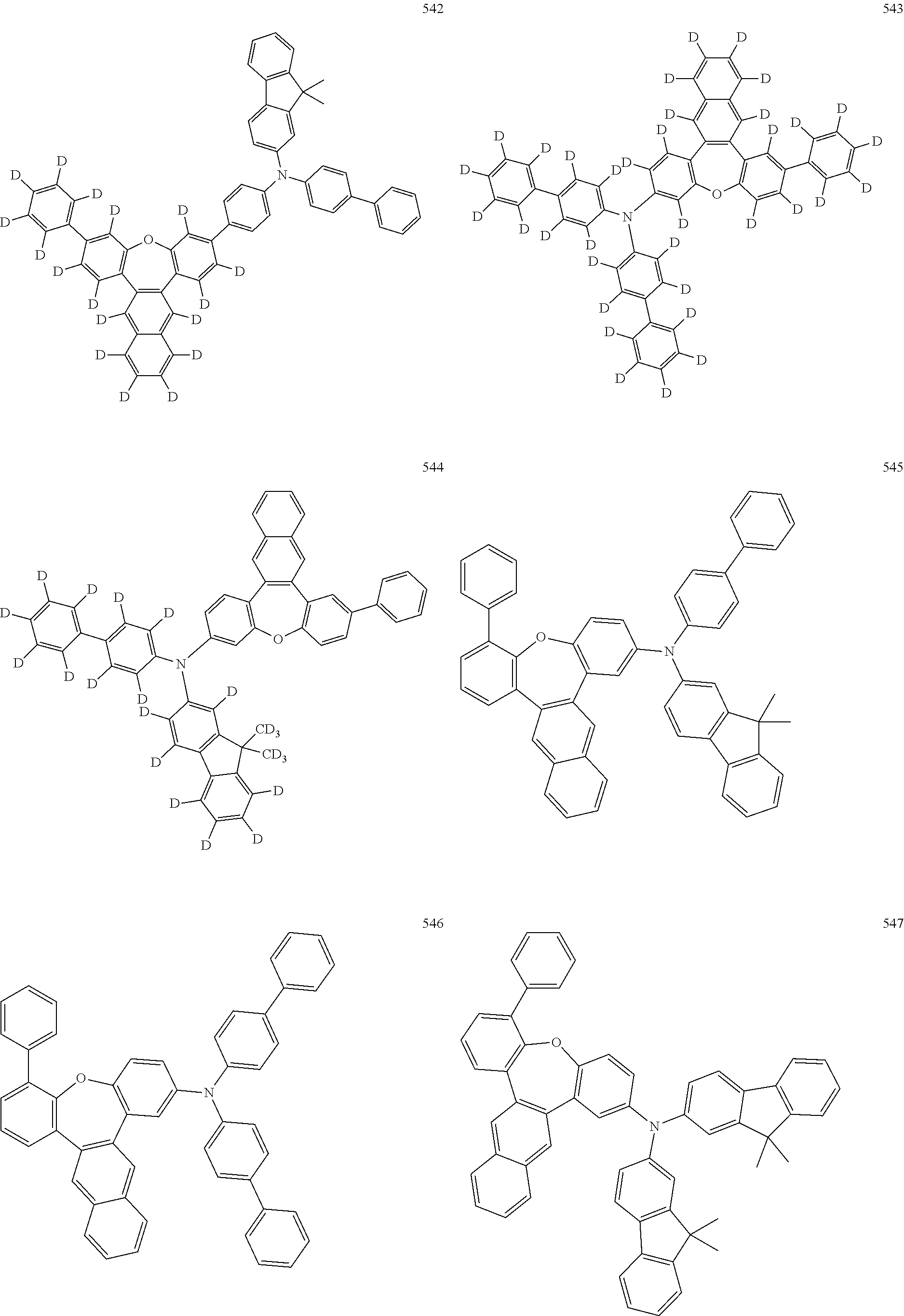

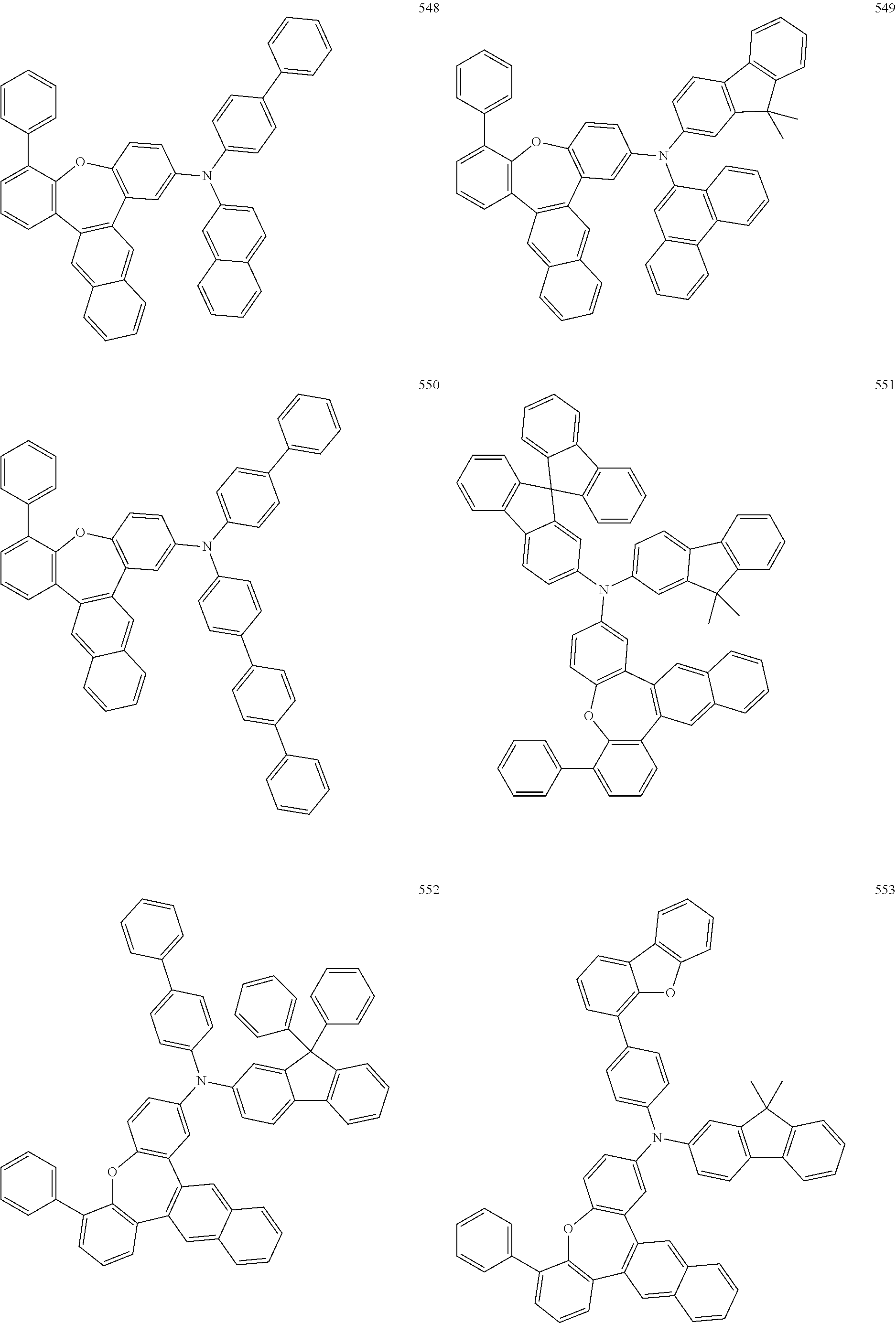

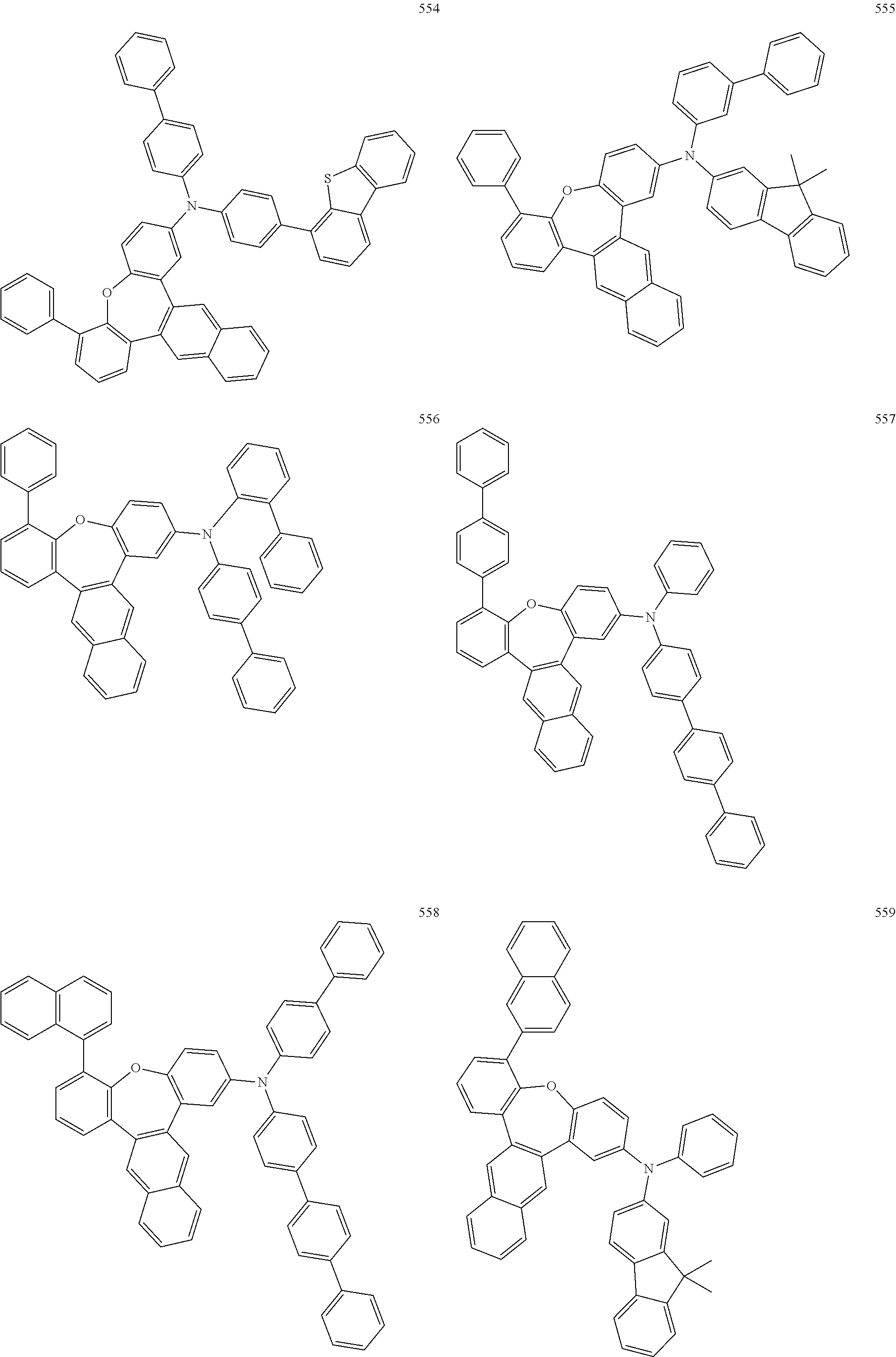

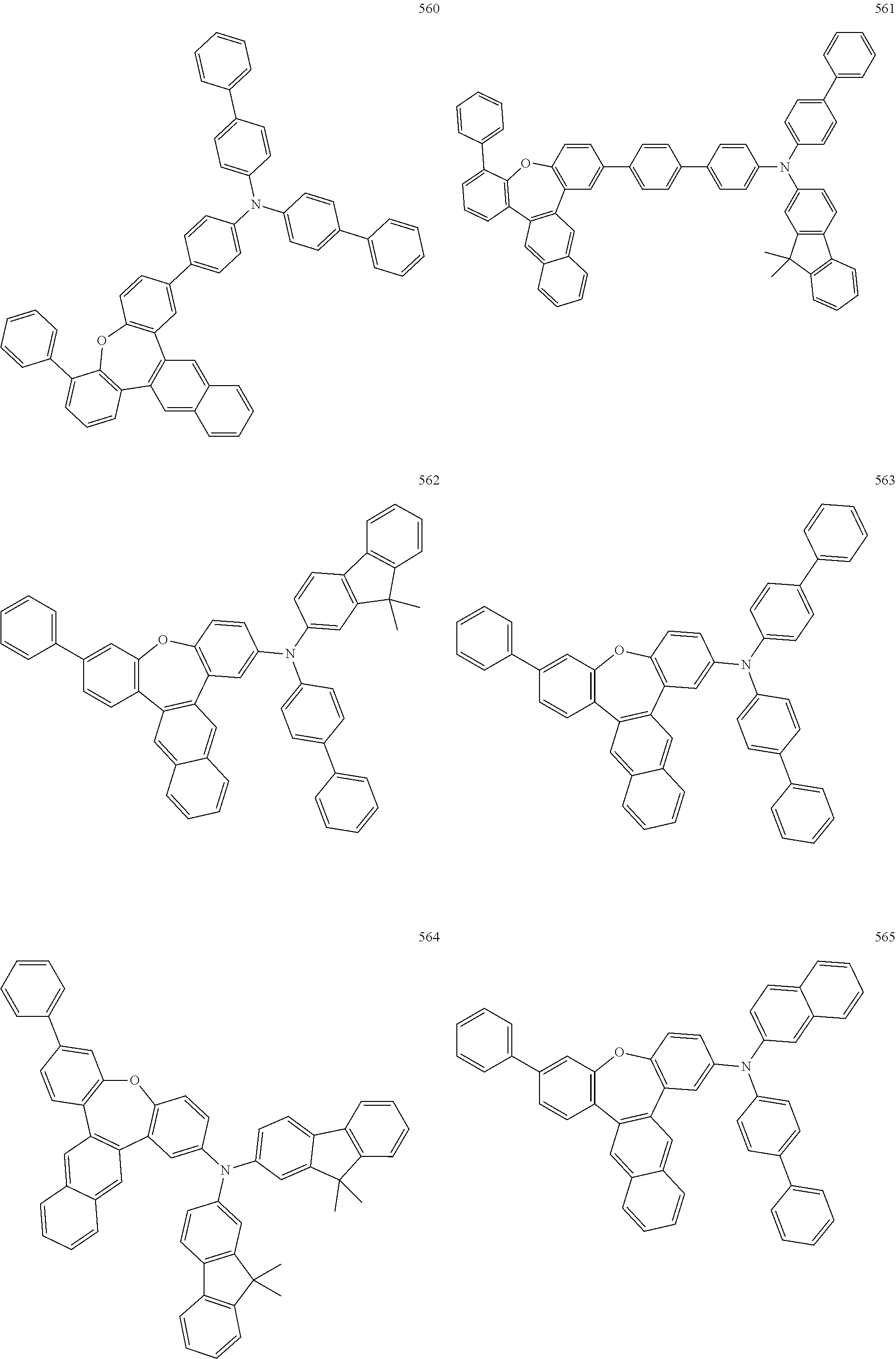

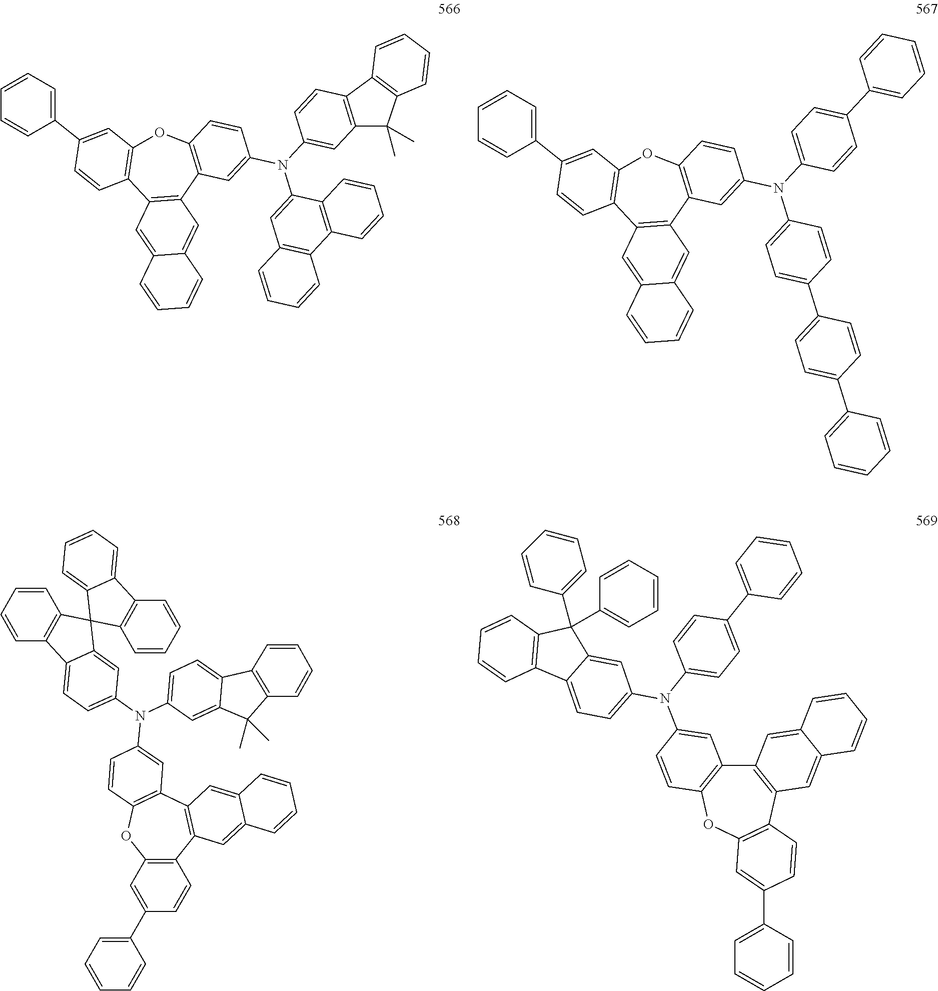 [SMILES: CC1(C)c2ccccc2-c2ccc(N(c3ccc4c(c3)-c3cc5ccccc5cc3-c3ccc(-c5ccccc5)cc3O4)c3cc4ccccc4c4ccccc34)cc21 ; c1ccc(-c2ccc(-c3ccc(N(c4ccc(-c5ccccc5)cc4)c4ccc5c(c4)-c4cc6ccccc6cc4-c4ccc(-c6ccccc6)cc4O5)cc3)cc2)cc1 ; CC1(C)c2ccccc2-c2ccc(N(c3ccc4c(c3)-c3cc5ccccc5cc3-c3ccc(-c5ccccc5)cc3O4)c3ccc4c(c3)C3(c5ccccc5-c5ccccc53)c3ccccc3-4)cc21 ; c1ccc(-c2ccc(N(c3ccc4c(c3)-c3cc5ccccc5cc3-c3ccc(-c5ccccc5)cc3O4)c3ccc4c(c3)C(c3ccccc3)(c3ccccc3)c3ccccc3-4)cc2)cc1]

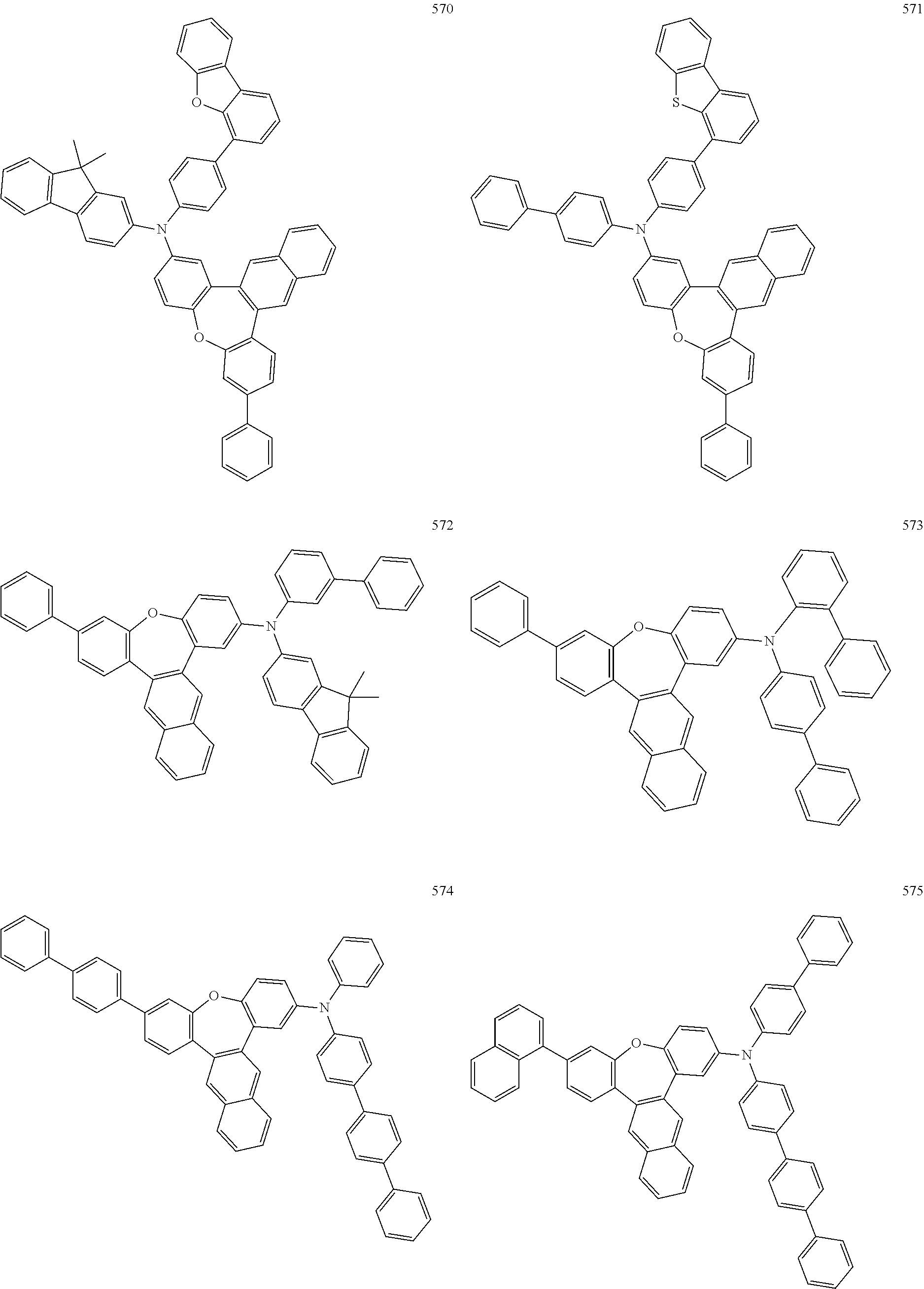

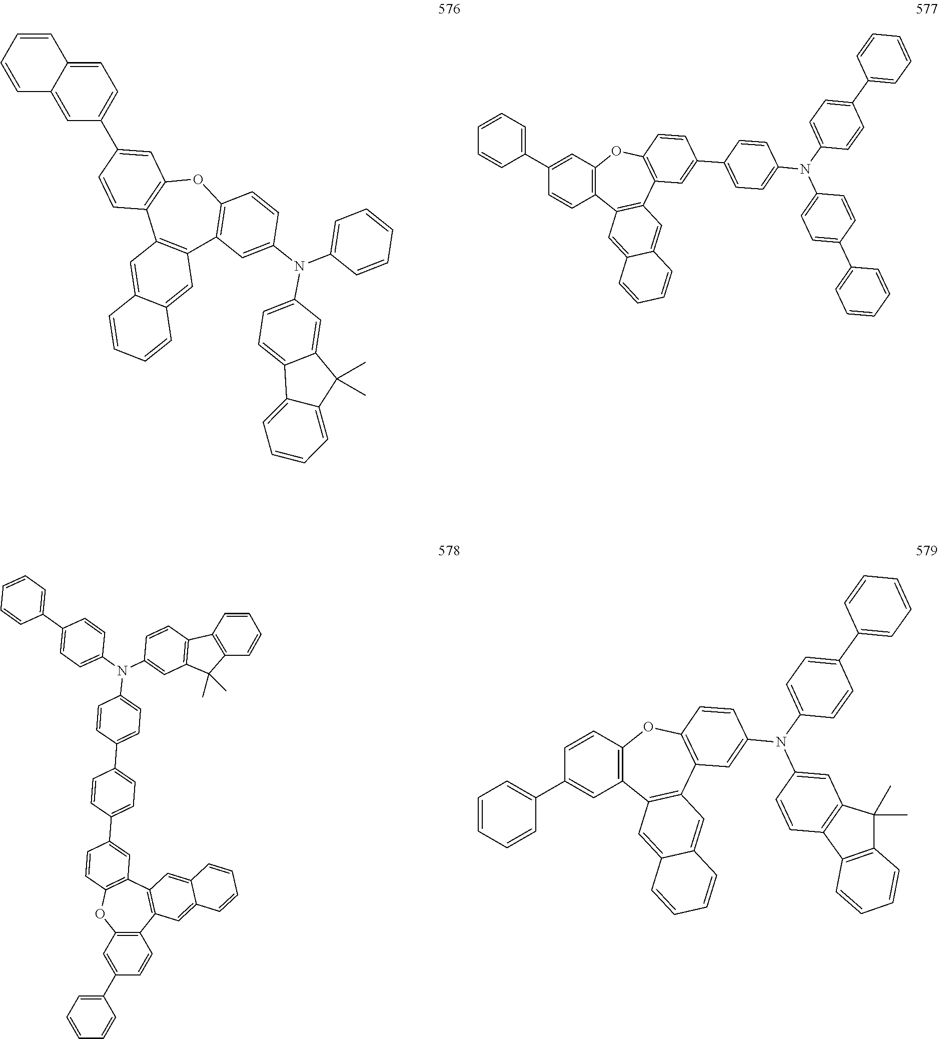 [SMILES: CC1(C)c2ccccc2-c2ccc(N(c3ccccc3)c3ccc4c(c3)-c3cc5ccccc5cc3-c3ccc(-c5ccc6ccccc6c5)cc3O4)cc21 ; c1ccc(-c2ccc(N(c3ccc(-c4ccccc4)cc3)c3ccc(-c4ccc5c(c4)-c4cc6ccccc6cc4-c4ccc(-c6ccccc6)cc4O5)cc3)cc2)cc1 ; CC1(C)c2ccccc2-c2ccc(N(c3ccc(-c4ccccc4)cc3)c3ccc(-c4ccc(-c5ccc6c(c5)-c5cc7ccccc7cc5-c5ccc(-c7ccccc7)cc5O6)cc4)cc3)cc21 ; CC1(C)c2ccccc2-c2ccc(N(c3ccc(-c4ccccc4)cc3)c3ccc4c(c3)-c3cc5ccccc5cc3-c3cc(-c5ccccc5)ccc3O4)cc21]

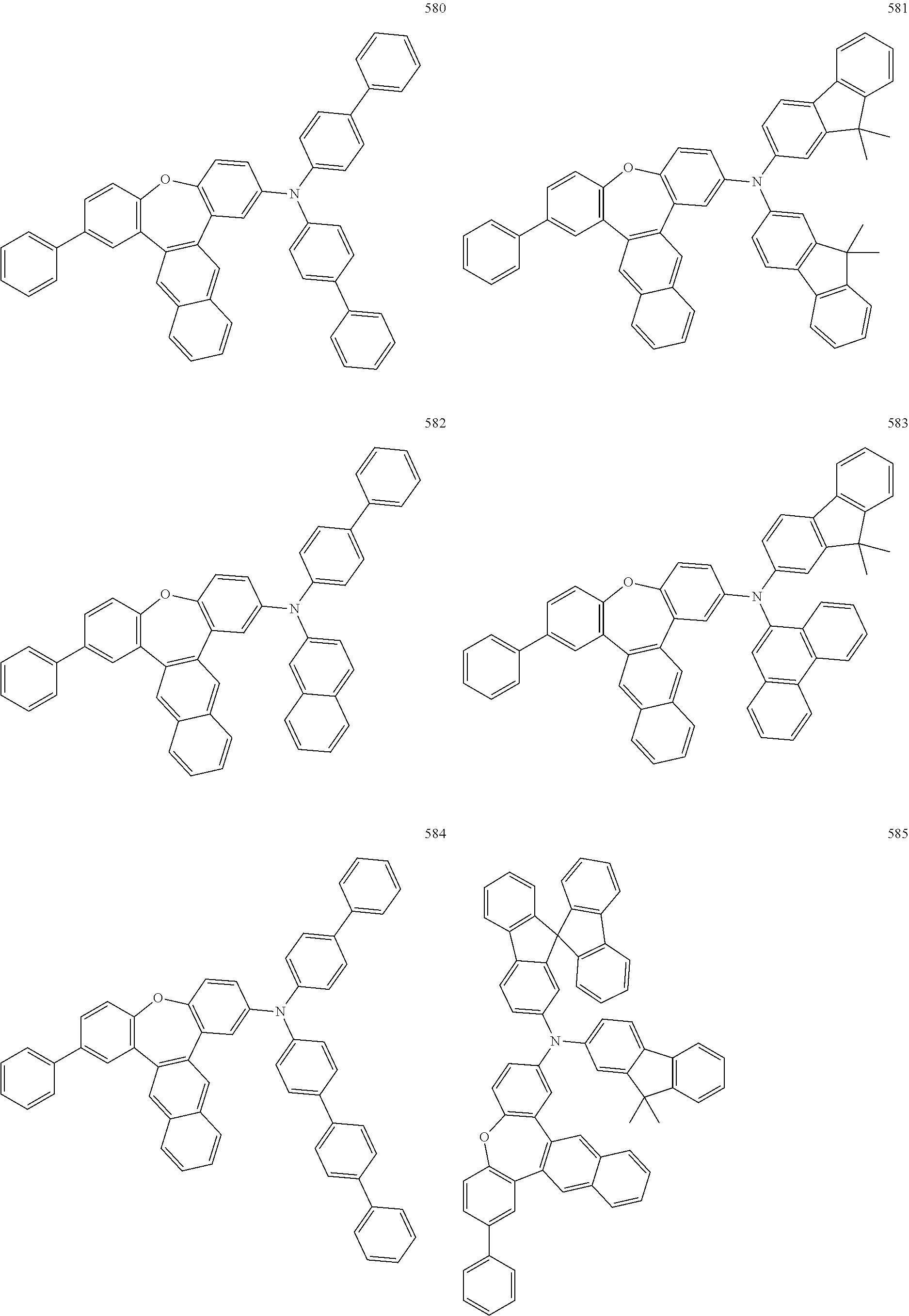

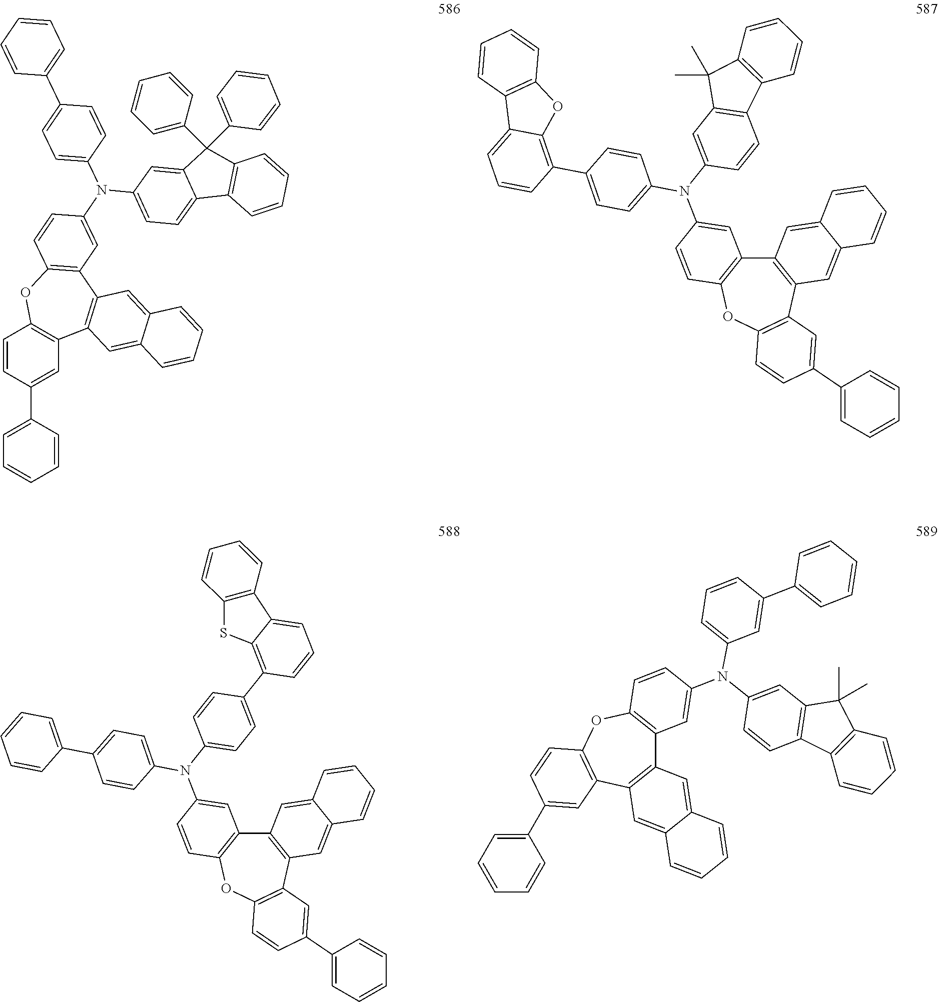 [SMILES: c1ccc(-c2ccc(N(c3ccc4c(c3)-c3cc5ccccc5cc3-c3cc(-c5ccccc5)ccc3O4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)-c3cc5ccccc5cc3-c3cc(-c5ccccc5)ccc3O4)cc21 ; c1ccc(-c2ccc(N(c3ccc(-c4cccc5c4sc4ccccc45)cc3)c3ccc4c(c3)-c3cc5ccccc5cc3-c3cc(-c5ccccc5)ccc3O4)cc2)cc1 ; CC1(C)c2ccccc2-c2ccc(N(c3cccc(-c4ccccc4)c3)c3ccc4c(c3)-c3cc5ccccc5cc3-c3cc(-c5ccccc5)ccc3O4)cc21]

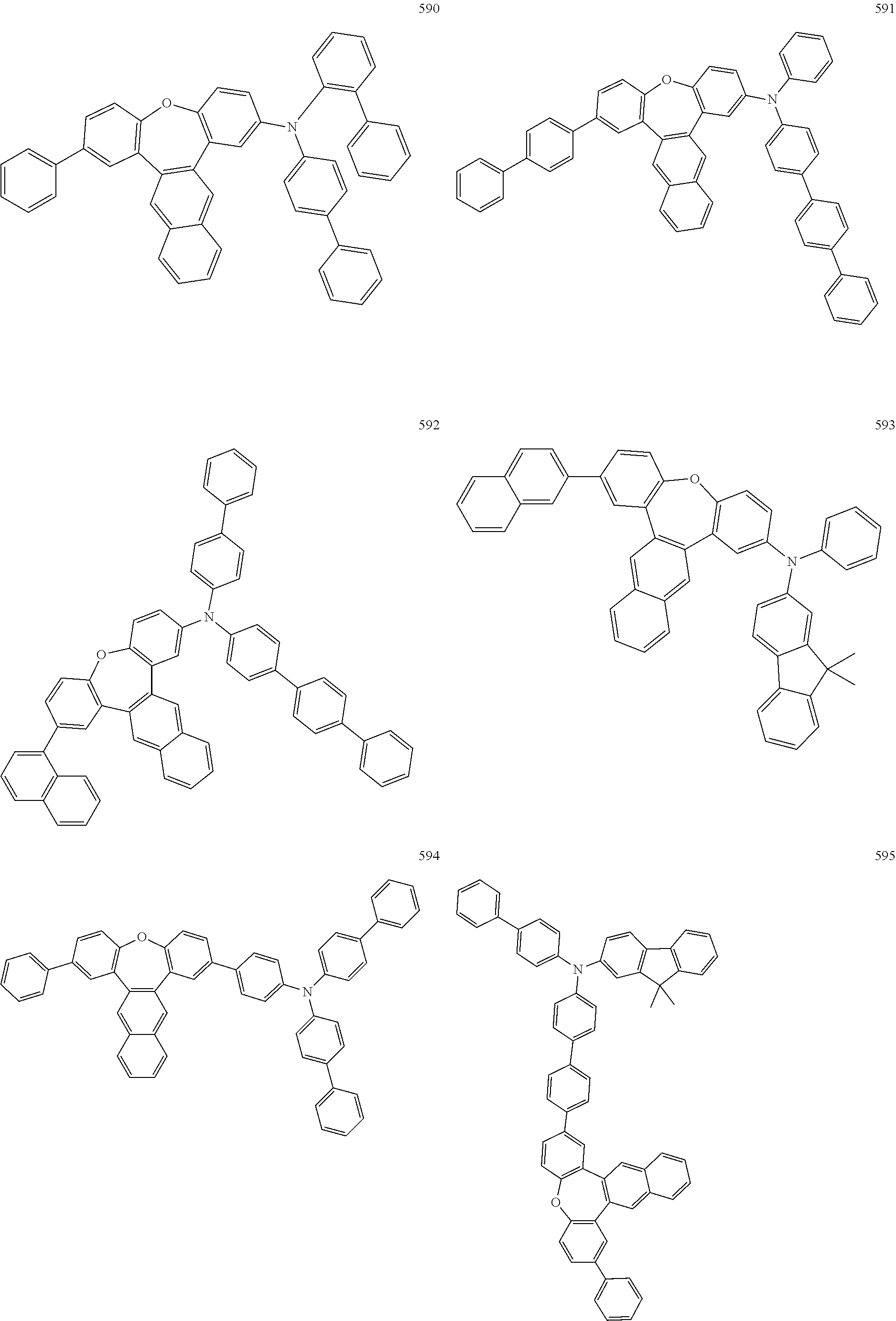

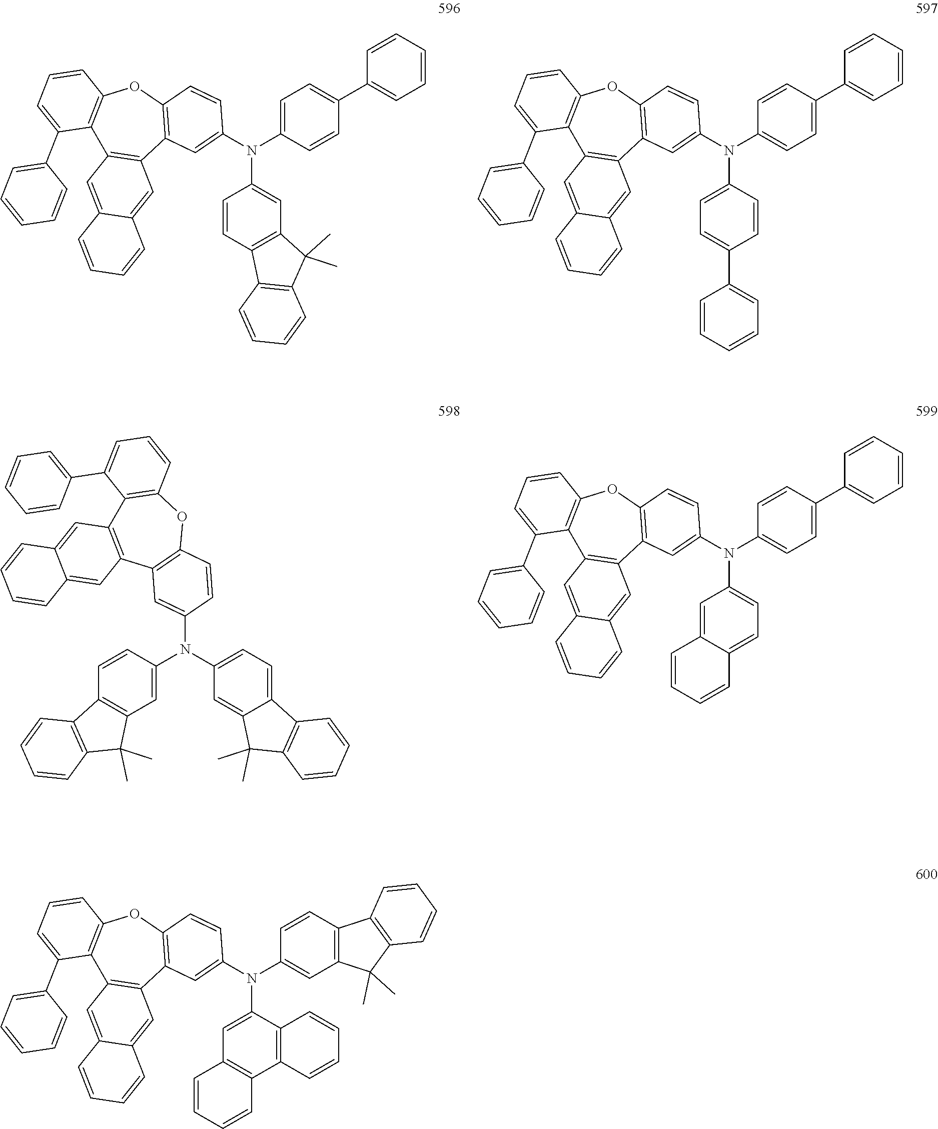 [SMILES: CC1(C)c2ccccc2-c2ccc(N(c3ccc(-c4ccccc4)cc3)c3ccc4c(c3)-c3cc5ccccc5cc3-c3c(cccc3-c3ccccc3)O4)cc21 ; c1ccc(-c2ccc(N(c3ccc(-c4ccccc4)cc3)c3ccc4c(c3)-c3cc5ccccc5cc3-c3c(cccc3-c3ccccc3)O4)cc2)cc1 ; CC1(C)c2ccccc2-c2ccc(N(c3ccc4c(c3)-c3cc5ccccc5cc3-c3c(cccc3-c3ccccc3)O4)c3ccc4c(c3)C(C)(C)c3ccccc3-4)cc21 ; c1ccc(-c2ccc(N(c3ccc4c(c3)-c3cc5ccccc5cc3-c3c(cccc3-c3ccccc3)O4)c3ccc4ccccc4c3)cc2)cc1 ; CC1(C)c2ccccc2-c2ccc(N(c3ccc4c(c3)-c3cc5ccccc5cc3-c3c(cccc3-c3ccccc3)O4)c3cc4ccccc4c4ccccc34)cc21]

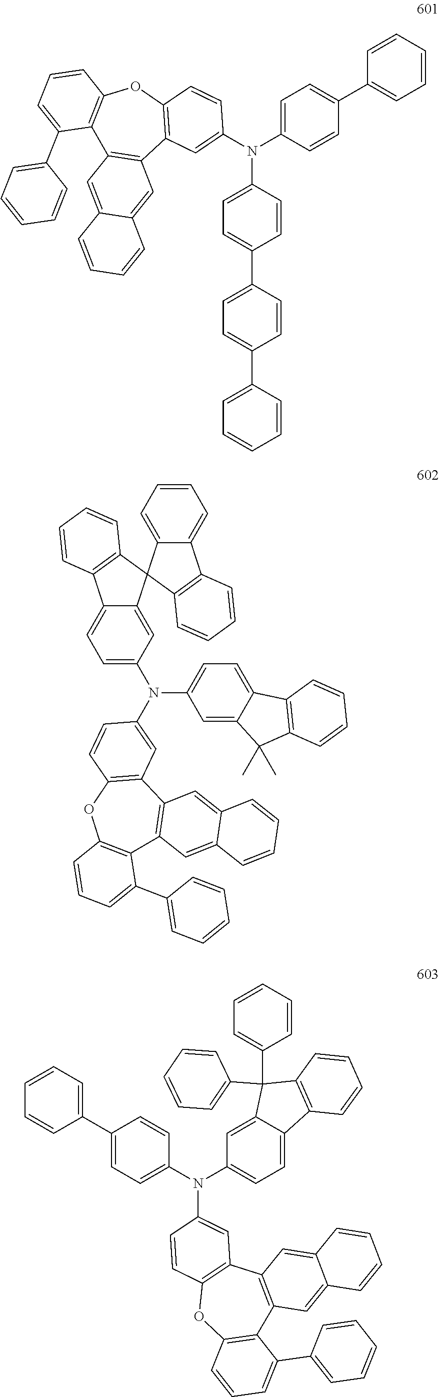 [SMILES: c1ccc(-c2ccc(-c3ccc(N(c4ccc(-c5ccccc5)cc4)c4ccc5c(c4)-c4cc6ccccc6cc4-c4c(cccc4-c4ccccc4)O5)cc3)cc2)cc1 ; CC1(C)c2ccccc2-c2ccc(N(c3ccc4c(c3)-c3cc5ccccc5cc3-c3c(cccc3-c3ccccc3)O4)c3ccc4c(c3)C3(c5ccccc5-c5ccccc53)c3ccccc3-4)cc21 ; c1ccc(-c2ccc(N(c3ccc4c(c3)-c3cc5ccccc5cc3-c3c(cccc3-c3ccccc3)O4)c3ccc4c(c3)C(c3ccccc3)(c3ccccc3)c3ccccc3-4)cc2)cc1]

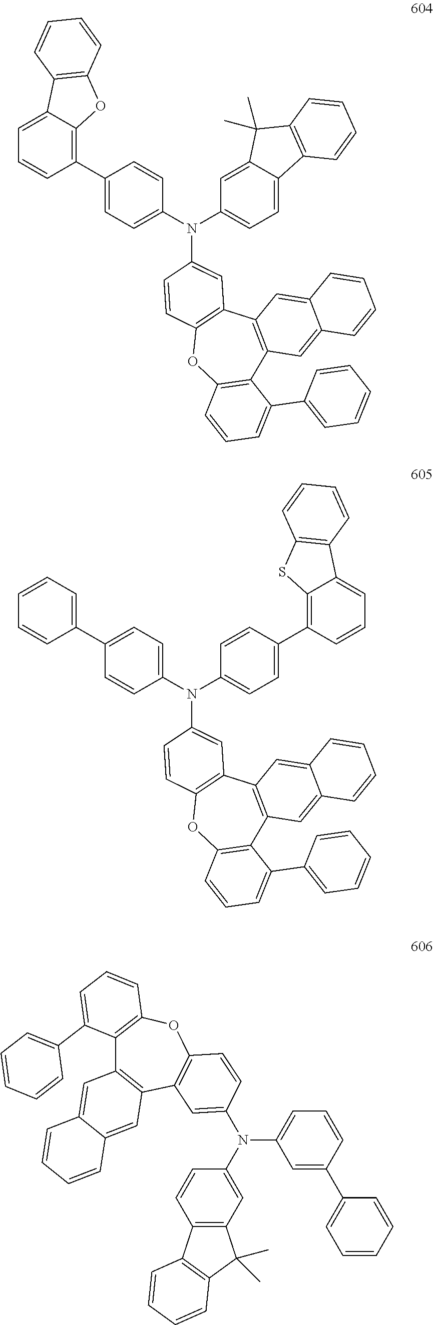 [SMILES: CC1(C)c2ccccc2-c2ccc(N(c3ccc(-c4cccc5c4oc4ccccc45)cc3)c3ccc4c(c3)-c3cc5ccccc5cc3-c3c(cccc3-c3ccccc3)O4)cc21 ; c1ccc(-c2ccc(N(c3ccc(-c4cccc5c4sc4ccccc45)cc3)c3ccc4c(c3)-c3cc5ccccc5cc3-c3c(cccc3-c3ccccc3)O4)cc2)cc1 ; CC1(C)c2ccccc2-c2ccc(N(c3cccc(-c4ccccc4)c3)c3ccc4c(c3)-c3cc5ccccc5cc3-c3c(cccc3-c3ccccc3)O4)cc21]

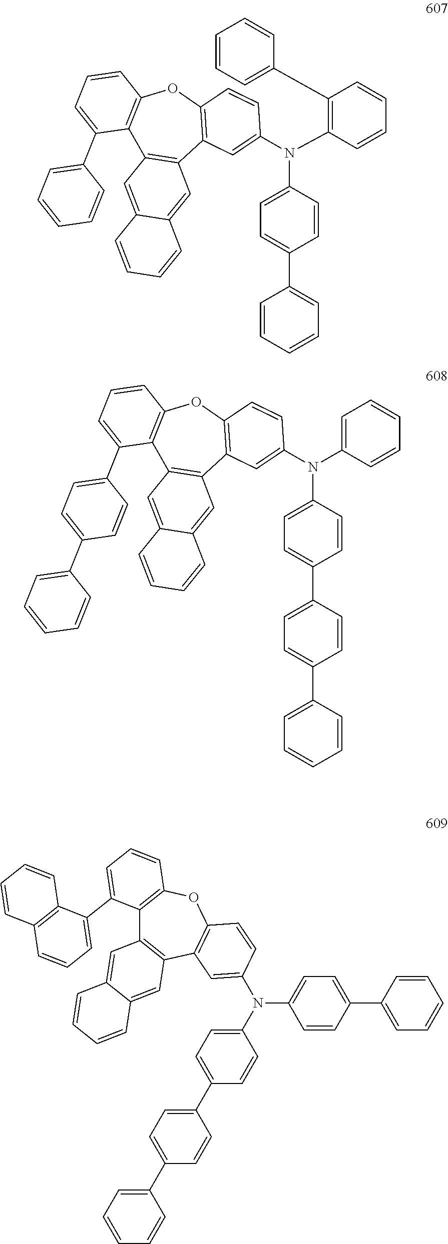 [SMILES: c1ccc(-c2ccc(N(c3ccc4c(c3)-c3cc5ccccc5cc3-c3c(cccc3-c3ccccc3)O4)c3ccccc3-c3ccccc3)cc2)cc1 ; c1ccc(-c2ccc(-c3ccc(N(c4ccccc4)c4ccc5c(c4)-c4cc6ccccc6cc4-c4c(cccc4-c4ccc(-c6ccccc6)cc4)O5)cc3)cc2)cc1 ; c1ccc(-c2ccc(-c3ccc(N(c4ccc(-c5ccccc5)cc4)c4ccc5c(c4)-c4cc6ccccc6cc4-c4c(cccc4-c4cccc6ccccc46)O5)cc3)cc2)cc1]

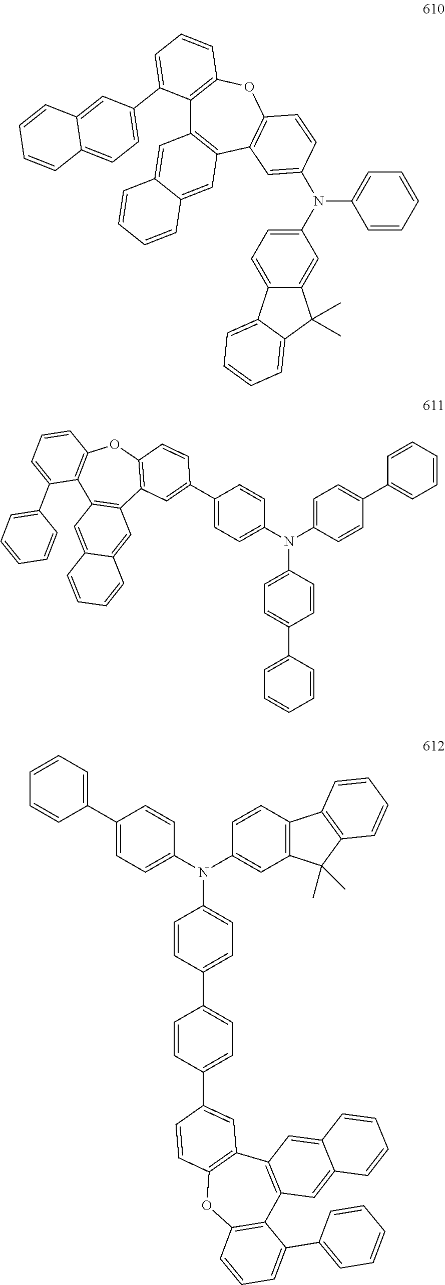 [SMILES: CC1(C)c2ccccc2-c2ccc(N(c3ccccc3)c3ccc4c(c3)-c3cc5ccccc5cc3-c3c(cccc3-c3ccc5ccccc5c3)O4)cc21 ; c1ccc(-c2ccc(N(c3ccc(-c4ccccc4)cc3)c3ccc(-c4ccc5c(c4)-c4cc6ccccc6cc4-c4c(cccc4-c4ccccc4)O5)cc3)cc2)cc1 ; CC1(C)c2ccccc2-c2ccc(N(c3ccc(-c4ccccc4)cc3)c3ccc(-c4ccc(-c5ccc6c(c5)-c5cc7ccccc7cc5-c5c(cccc5-c5ccccc5)O6)cc4)cc3)cc21]

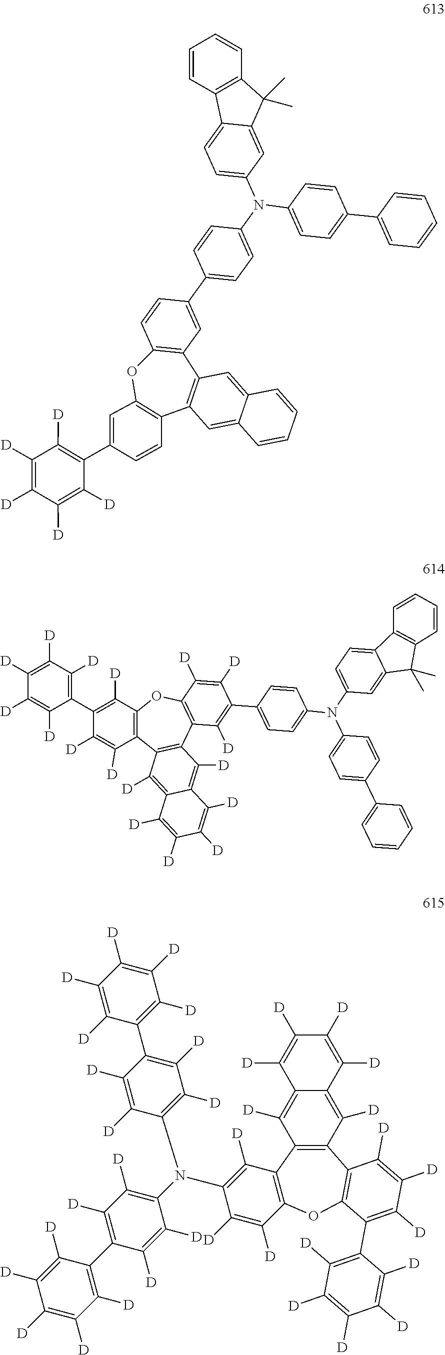

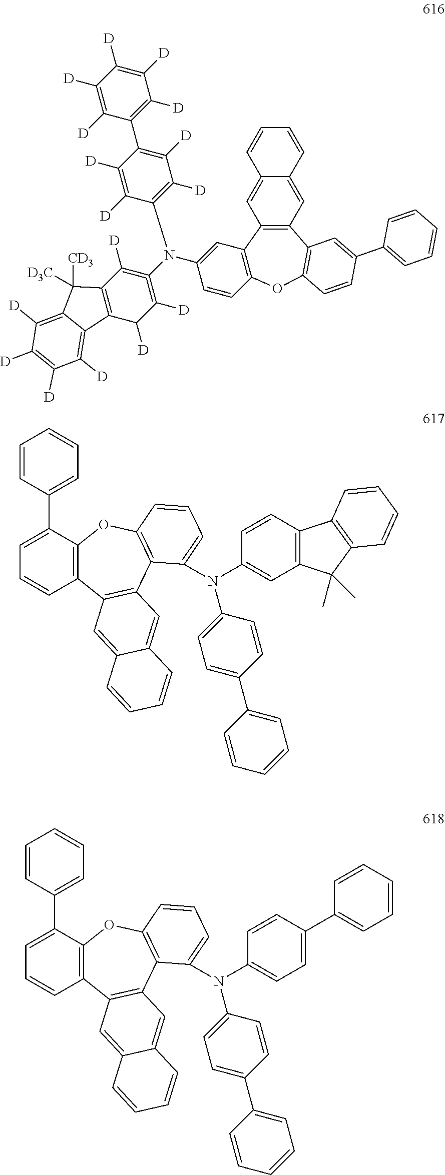

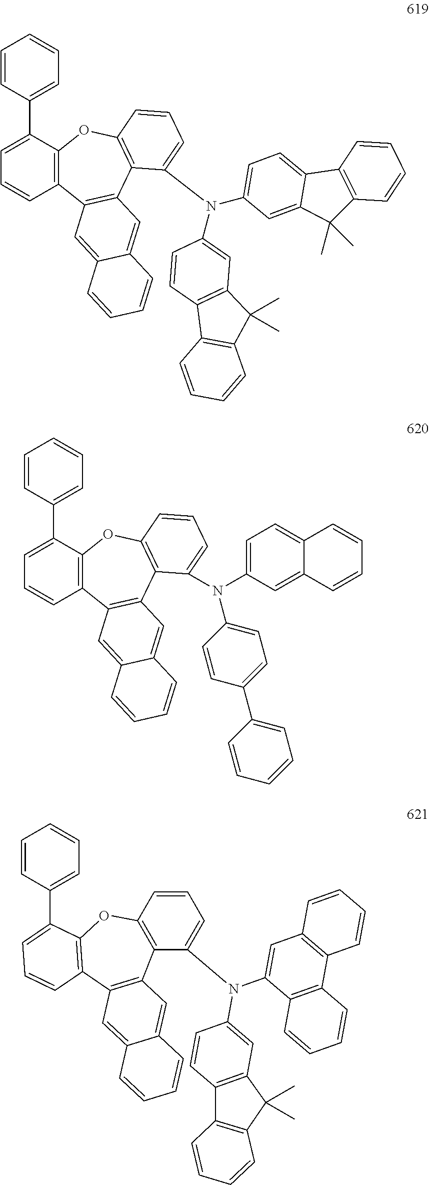 [SMILES: CC1(C)c2ccccc2-c2ccc(N(c3ccc4c(c3)C(C)(C)c3ccccc3-4)c3cccc4c3-c3cc5ccccc5cc3-c3cccc(-c5ccccc5)c3O4)cc21 ; c1ccc(-c2ccc(N(c3ccc4ccccc4c3)c3cccc4c3-c3cc5ccccc5cc3-c3cccc(-c5ccccc5)c3O4)cc2)cc1 ; CC1(C)c2ccccc2-c2ccc(N(c3cccc4c3-c3cc5ccccc5cc3-c3cccc(-c5ccccc5)c3O4)c3cc4ccccc4c4ccccc34)cc21]

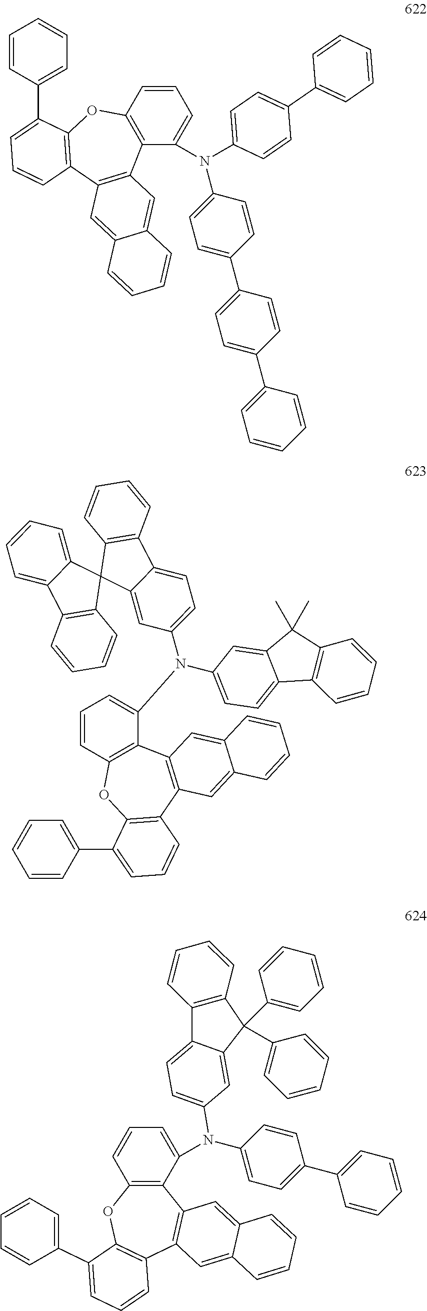 [SMILES: c1ccc(-c2ccc(-c3ccc(N(c4ccc(-c5ccccc5)cc4)c4cccc5c4-c4cc6ccccc6cc4-c4cccc(-c6ccccc6)c4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3cc5ccccc5cc3-c3cccc(-c5ccccc5)c3O4)cc21 ; c1ccc(-c2ccc(N(c3ccc4c(c3)C(c3ccccc3)(c3ccccc3)c3ccccc3-4)c3cccc4c3-c3cc5ccccc5cc3-c3cccc(-c5ccccc5)c3O4)cc2)cc1]

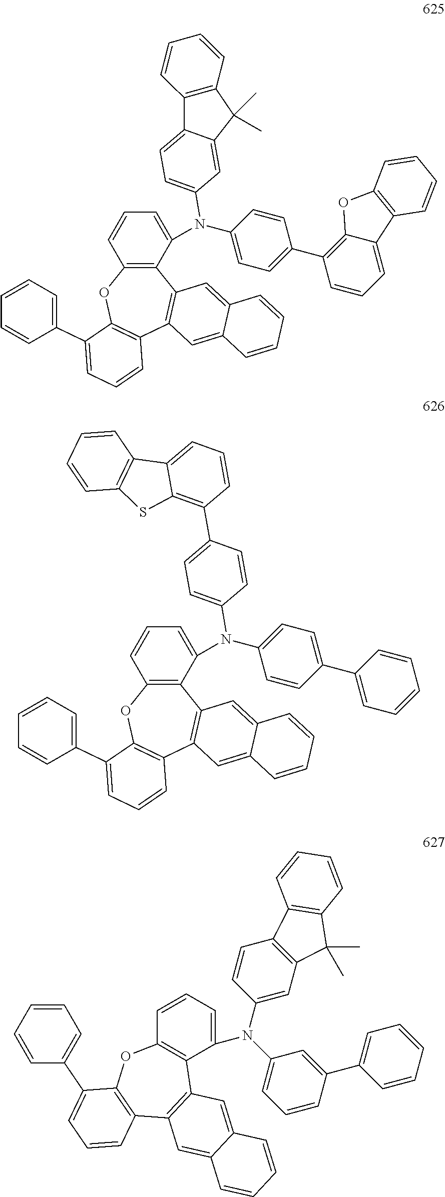

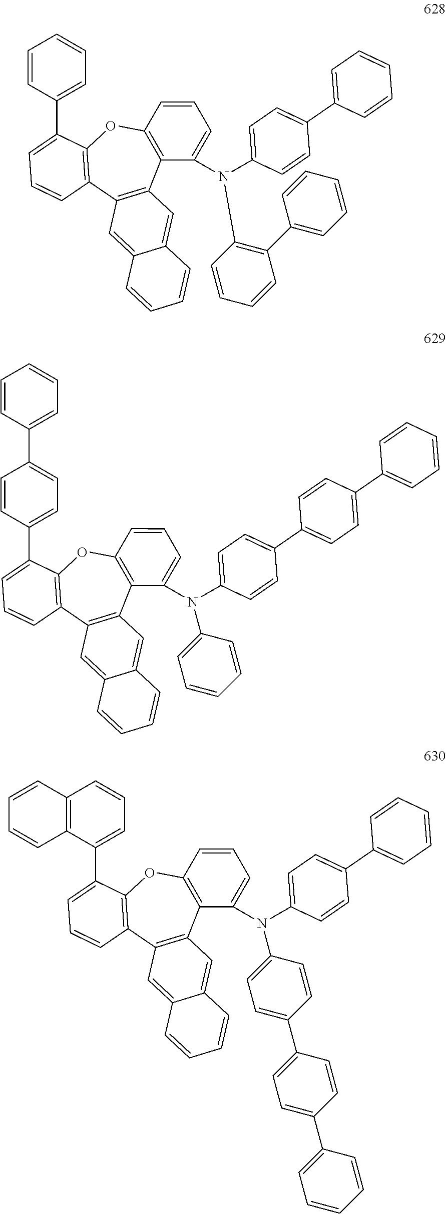 [SMILES: c1ccc(-c2ccc(N(c3ccccc3-c3ccccc3)c3cccc4c3-c3cc5ccccc5cc3-c3cccc(-c5ccccc5)c3O4)cc2)cc1 ; c1ccc(-c2ccc(-c3ccc(N(c4ccccc4)c4cccc5c4-c4cc6ccccc6cc4-c4cccc(-c6ccc(-c7ccccc7)cc6)c4O5)cc3)cc2)cc1 ; c1ccc(-c2ccc(-c3ccc(N(c4ccc(-c5ccccc5)cc4)c4cccc5c4-c4cc6ccccc6cc4-c4cccc(-c6cccc7ccccc67)c4O5)cc3)cc2)cc1]

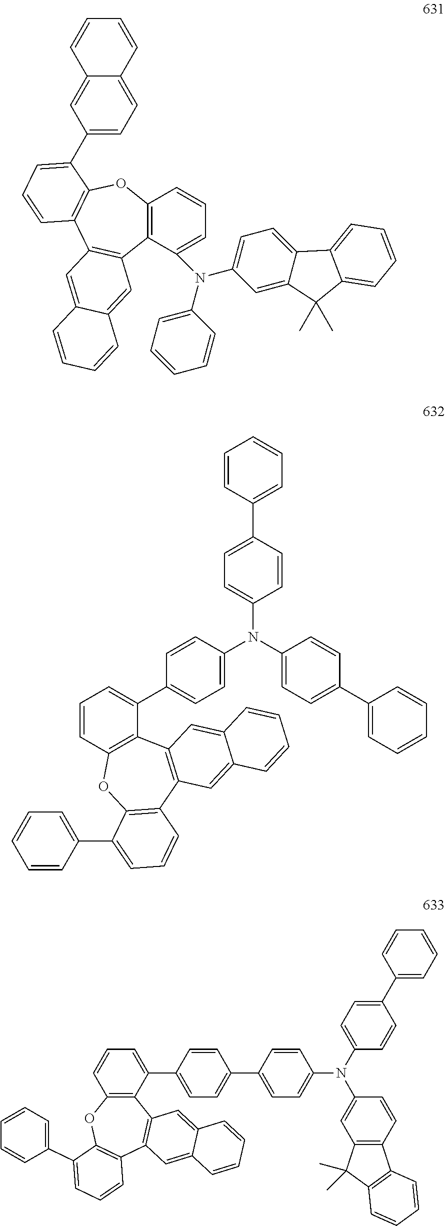 [SMILES: CC1(C)c2ccccc2-c2ccc(N(c3ccccc3)c3cccc4c3-c3cc5ccccc5cc3-c3cccc(-c5ccc6ccccc6c5)c3O4)cc21 ; c1ccc(-c2ccc(N(c3ccc(-c4ccccc4)cc3)c3ccc(-c4cccc5c4-c4cc6ccccc6cc4-c4cccc(-c6ccccc6)c4O5)cc3)cc2)cc1 ; CC1(C)c2ccccc2-c2ccc(N(c3ccc(-c4ccccc4)cc3)c3ccc(-c4ccc(-c5cccc6c5-c5cc7ccccc7cc5-c5cccc(-c7ccccc7)c5O6)cc4)cc3)cc21]

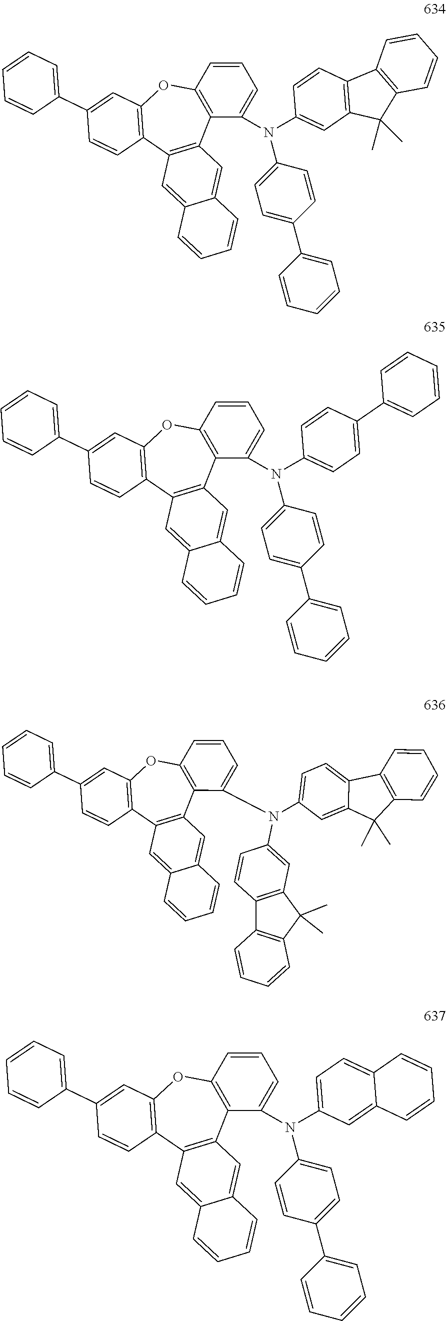 [SMILES: CC1(C)c2ccccc2-c2ccc(N(c3ccc(-c4ccccc4)cc3)c3cccc4c3-c3cc5ccccc5cc3-c3ccc(-c5ccccc5)cc3O4)cc21 ; c1ccc(-c2ccc(N(c3ccc(-c4ccccc4)cc3)c3cccc4c3-c3cc5ccccc5cc3-c3ccc(-c5ccccc5)cc3O4)cc2)cc1 ; CC1(C)c2ccccc2-c2ccc(N(c3ccc4c(c3)C(C)(C)c3ccccc3-4)c3cccc4c3-c3cc5ccccc5cc3-c3ccc(-c5ccccc5)cc3O4)cc21 ; c1ccc(-c2ccc(N(c3ccc4ccccc4c3)c3cccc4c3-c3cc5ccccc5cc3-c3ccc(-c5ccccc5)cc3O4)cc2)cc1]

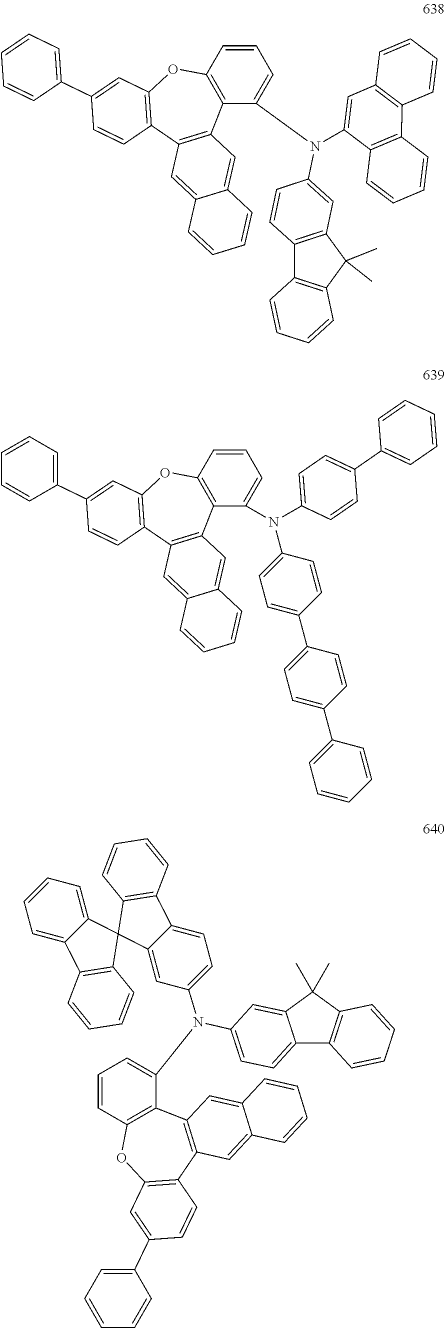 [SMILES: CC1(C)c2ccccc2-c2ccc(N(c3cccc4c3-c3cc5ccccc5cc3-c3ccc(-c5ccccc5)cc3O4)c3cc4ccccc4c4ccccc34)cc21 ; c1ccc(-c2ccc(-c3ccc(N(c4ccc(-c5ccccc5)cc4)c4cccc5c4-c4cc6ccccc6cc4-c4ccc(-c6ccccc6)cc4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3cc5ccccc5cc3-c3ccc(-c5ccccc5)cc3O4)cc21]

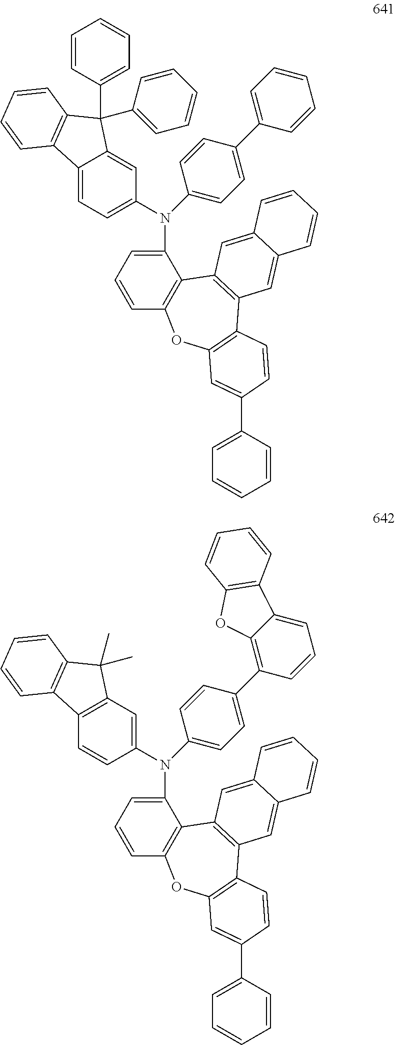

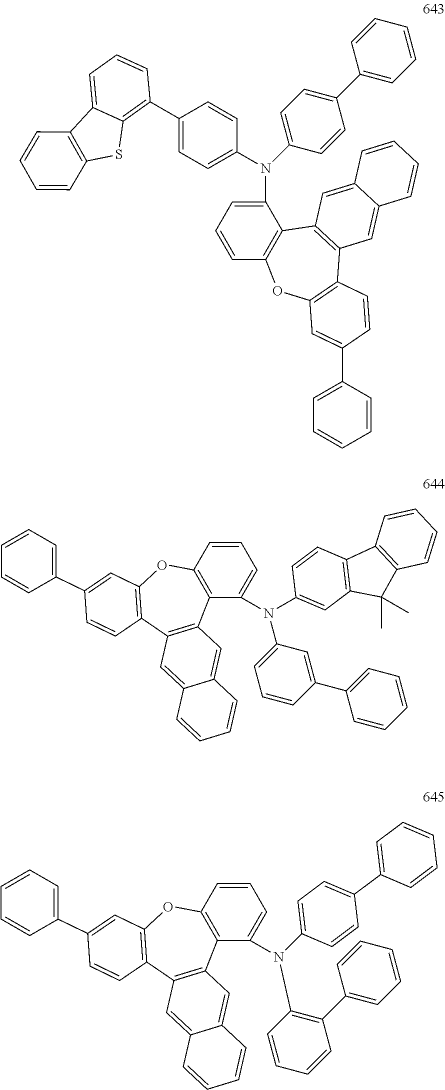 [SMILES: c1ccc(-c2ccc(N(c3ccc(-c4cccc5c4sc4ccccc45)cc3)c3cccc4c3-c3cc5ccccc5cc3-c3ccc(-c5ccccc5)cc3O4)cc2)cc1 ; CC1(C)c2ccccc2-c2ccc(N(c3cccc(-c4ccccc4)c3)c3cccc4c3-c3cc5ccccc5cc3-c3ccc(-c5ccccc5)cc3O4)cc21 ; c1ccc(-c2ccc(N(c3ccccc3-c3ccccc3)c3cccc4c3-c3cc5ccccc5cc3-c3ccc(-c5ccccc5)cc3O4)cc2)cc1]

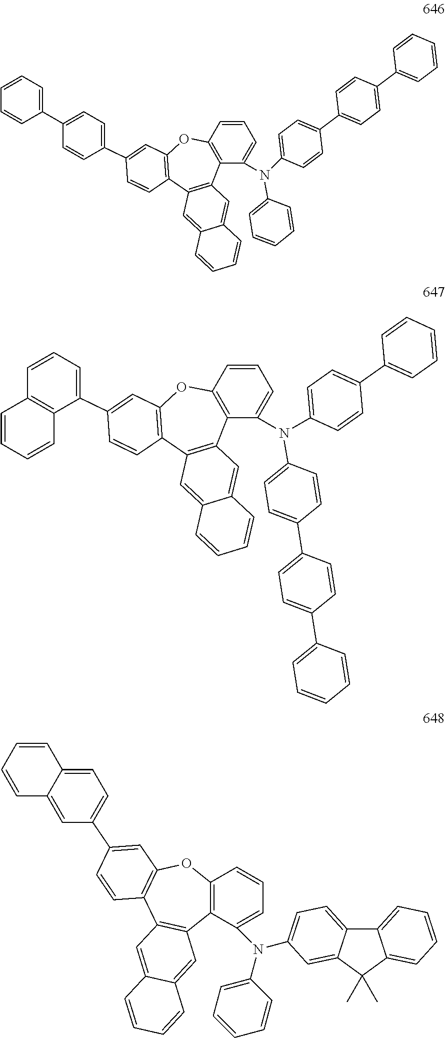 [SMILES: c1ccc(-c2ccc(-c3ccc(N(c4ccccc4)c4cccc5c4-c4cc6ccccc6cc4-c4ccc(-c6ccc(-c7ccccc7)cc6)cc4O5)cc3)cc2)cc1 ; c1ccc(-c2ccc(-c3ccc(N(c4ccc(-c5ccccc5)cc4)c4cccc5c4-c4cc6ccccc6cc4-c4ccc(-c6cccc7ccccc67)cc4O5)cc3)cc2)cc1 ; CC1(C)c2ccccc2-c2ccc(N(c3ccccc3)c3cccc4c3-c3cc5ccccc5cc3-c3ccc(-c5ccc6ccccc6c5)cc3O4)cc21]

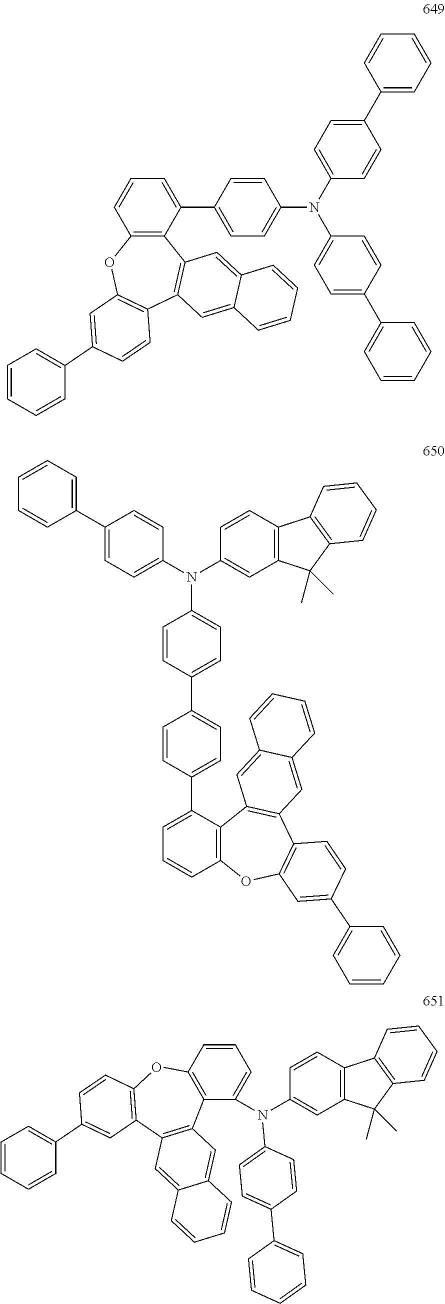

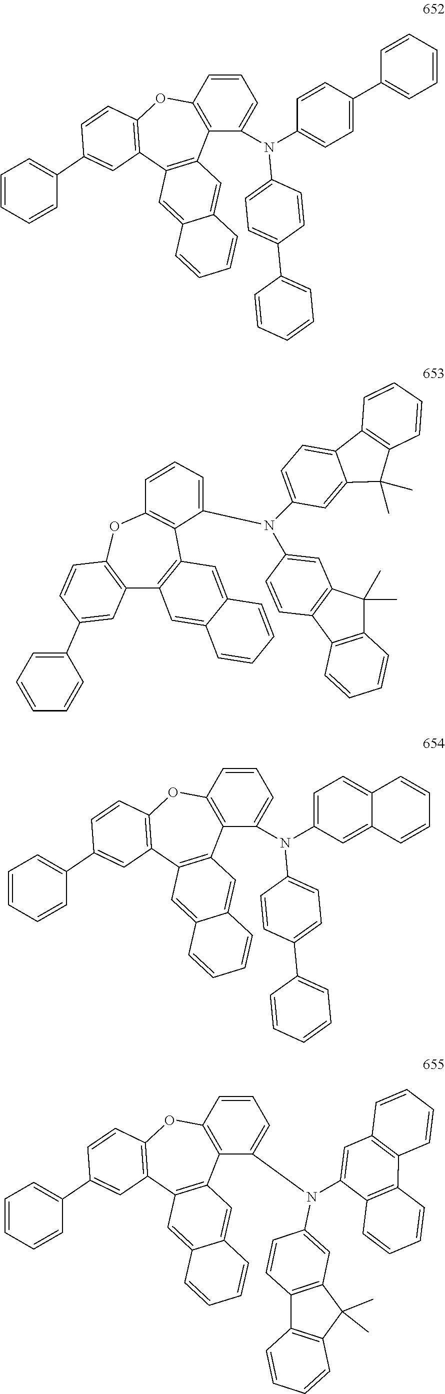 [SMILES: c1ccc(-c2ccc(N(c3ccc(-c4ccccc4)cc3)c3cccc4c3-c3cc5ccccc5cc3-c3cc(-c5ccccc5)ccc3O4)cc2)cc1 ; CC1(C)c2ccccc2-c2ccc(N(c3ccc4c(c3)C(C)(C)c3ccccc3-4)c3cccc4c3-c3cc5ccccc5cc3-c3cc(-c5ccccc5)ccc3O4)cc21 ; c1ccc(-c2ccc(N(c3ccc4ccccc4c3)c3cccc4c3-c3cc5ccccc5cc3-c3cc(-c5ccccc5)ccc3O4)cc2)cc1 ; CC1(C)c2ccccc2-c2ccc(N(c3cccc4c3-c3cc5ccccc5cc3-c3cc(-c5ccccc5)ccc3O4)c3cc4ccccc4c4ccccc34)cc21]

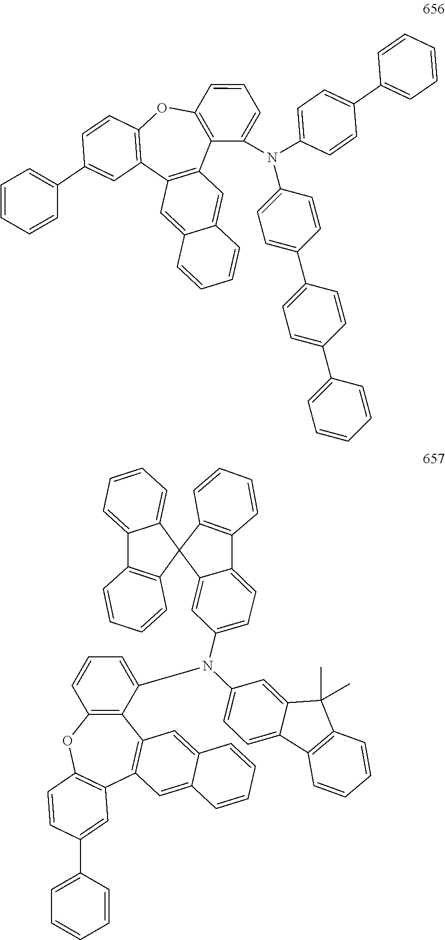 [SMILES: c1ccc(-c2ccc(-c3ccc(N(c4ccc(-c5ccccc5)cc4)c4cccc5c4-c4cc6ccccc6cc4-c4cc(-c6ccccc6)ccc4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3cc5ccccc5cc3-c3cc(-c5ccccc5)ccc3O4)cc21]

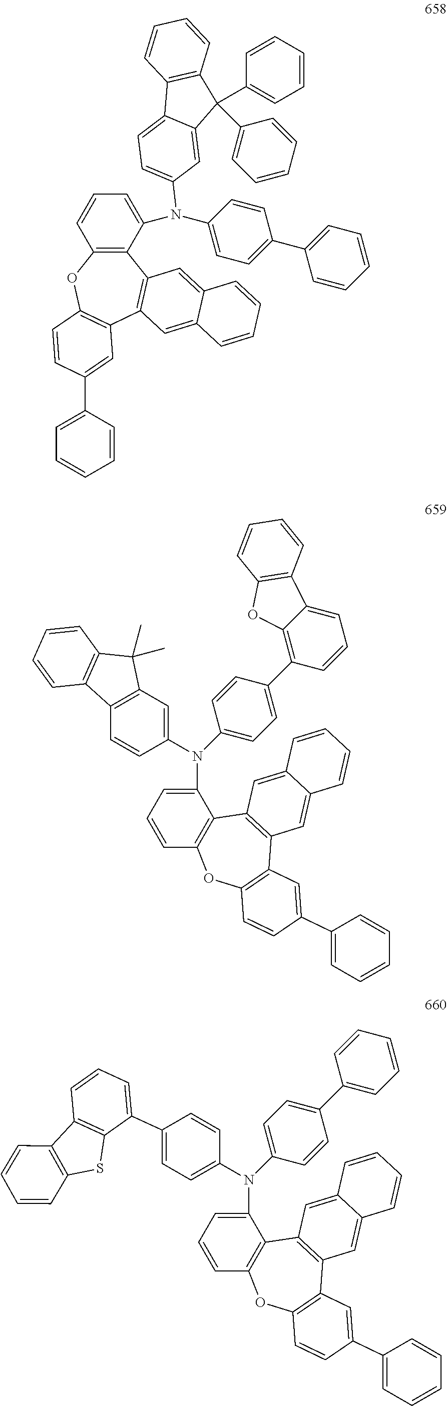 [SMILES: c1ccc(-c2ccc(N(c3ccc4c(c3)C(c3ccccc3)(c3ccccc3)c3ccccc3-4)c3cccc4c3-c3cc5ccccc5cc3-c3cc(-c5ccccc5)ccc3O4)cc2)cc1 ; CC1(C)c2ccccc2-c2ccc(N(c3ccc(-c4cccc5c4oc4ccccc45)cc3)c3cccc4c3-c3cc5ccccc5cc3-c3cc(-c5ccccc5)ccc3O4)cc21 ; c1ccc(-c2ccc(N(c3ccc(-c4cccc5c4sc4ccccc45)cc3)c3cccc4c3-c3cc5ccccc5cc3-c3cc(-c5ccccc5)ccc3O4)cc2)cc1]

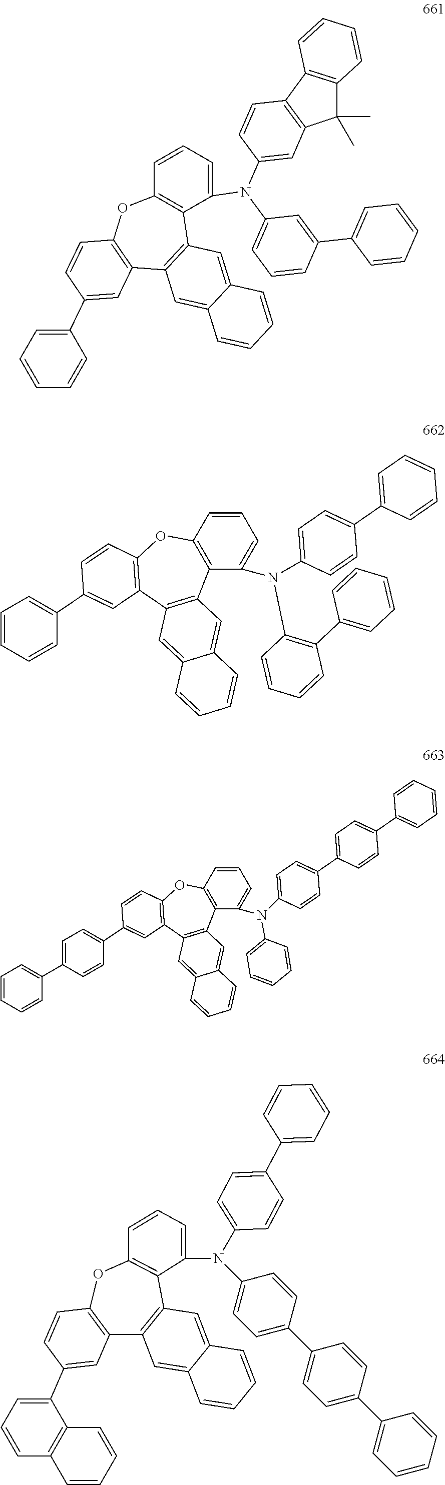 [SMILES: CC1(C)c2ccccc2-c2ccc(N(c3cccc(-c4ccccc4)c3)c3cccc4c3-c3cc5ccccc5cc3-c3cc(-c5ccccc5)ccc3O4)cc21 ; c1ccc(-c2ccc(N(c3ccccc3-c3ccccc3)c3cccc4c3-c3cc5ccccc5cc3-c3cc(-c5ccccc5)ccc3O4)cc2)cc1 ; c1ccc(-c2ccc(-c3ccc(N(c4ccccc4)c4cccc5c4-c4cc6ccccc6cc4-c4cc(-c6ccc(-c7ccccc7)cc6)ccc4O5)cc3)cc2)cc1 ; c1ccc(-c2ccc(-c3ccc(N(c4ccc(-c5ccccc5)cc4)c4cccc5c4-c4cc6ccccc6cc4-c4cc(-c6cccc7ccccc67)ccc4O5)cc3)cc2)cc1]

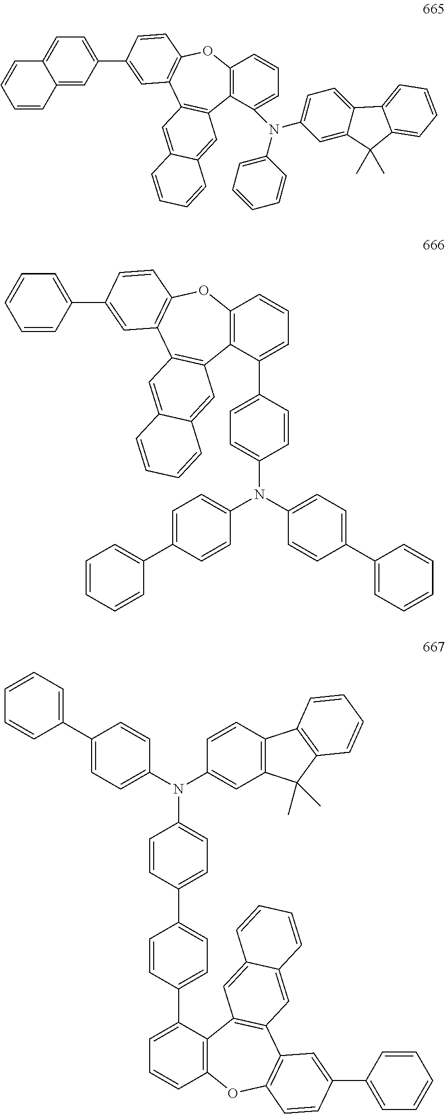 [SMILES: CC1(C)c2ccccc2-c2ccc(N(c3ccccc3)c3cccc4c3-c3cc5ccccc5cc3-c3cc(-c5ccc6ccccc6c5)ccc3O4)cc21 ; c1ccc(-c2ccc(N(c3ccc(-c4ccccc4)cc3)c3ccc(-c4cccc5c4-c4cc6ccccc6cc4-c4cc(-c6ccccc6)ccc4O5)cc3)cc2)cc1 ; CC1(C)c2ccccc2-c2ccc(N(c3ccc(-c4ccccc4)cc3)c3ccc(-c4ccc(-c5cccc6c5-c5cc7ccccc7cc5-c5cc(-c7ccccc7)ccc5O6)cc4)cc3)cc21]

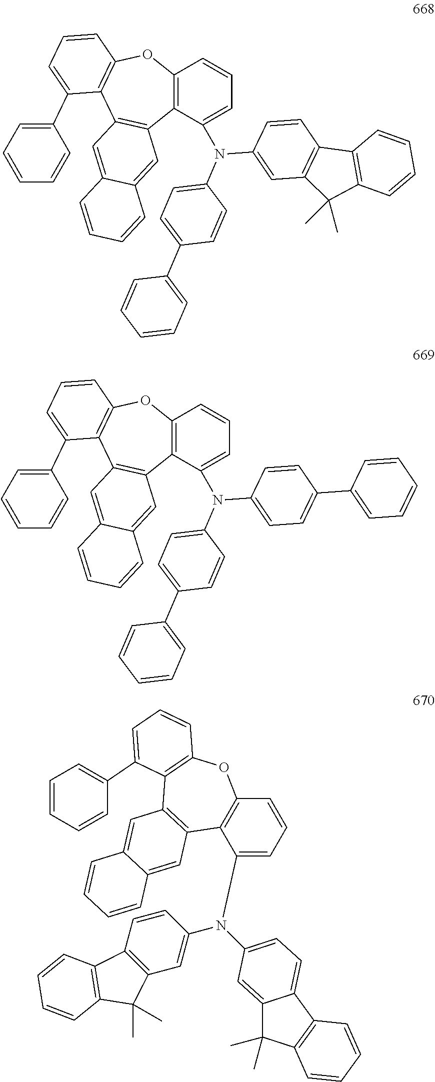

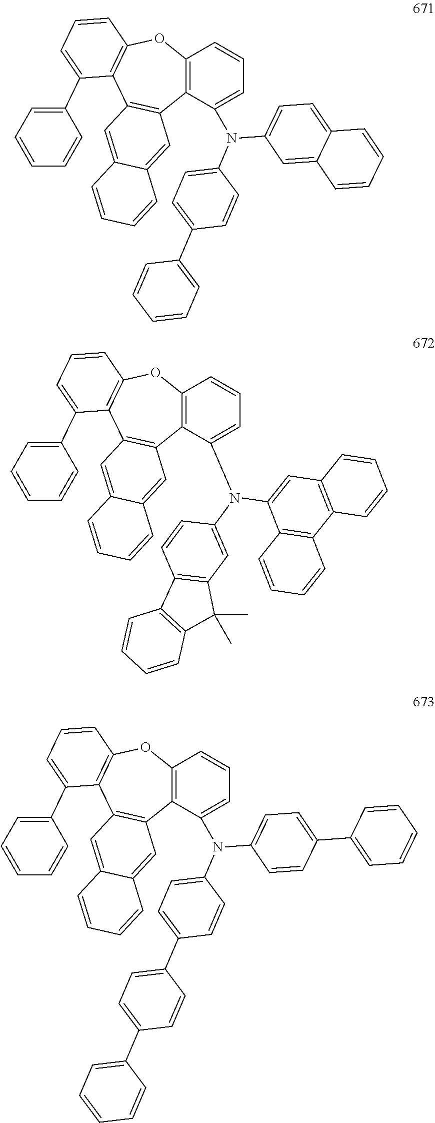 [SMILES: c1ccc(-c2ccc(N(c3ccc4ccccc4c3)c3cccc4c3-c3cc5ccccc5cc3-c3c(cccc3-c3ccccc3)O4)cc2)cc1 ; CC1(C)c2ccccc2-c2ccc(N(c3cccc4c3-c3cc5ccccc5cc3-c3c(cccc3-c3ccccc3)O4)c3cc4ccccc4c4ccccc34)cc21 ; c1ccc(-c2ccc(-c3ccc(N(c4ccc(-c5ccccc5)cc4)c4cccc5c4-c4cc6ccccc6cc4-c4c(cccc4-c4ccccc4)O5)cc3)cc2)cc1]

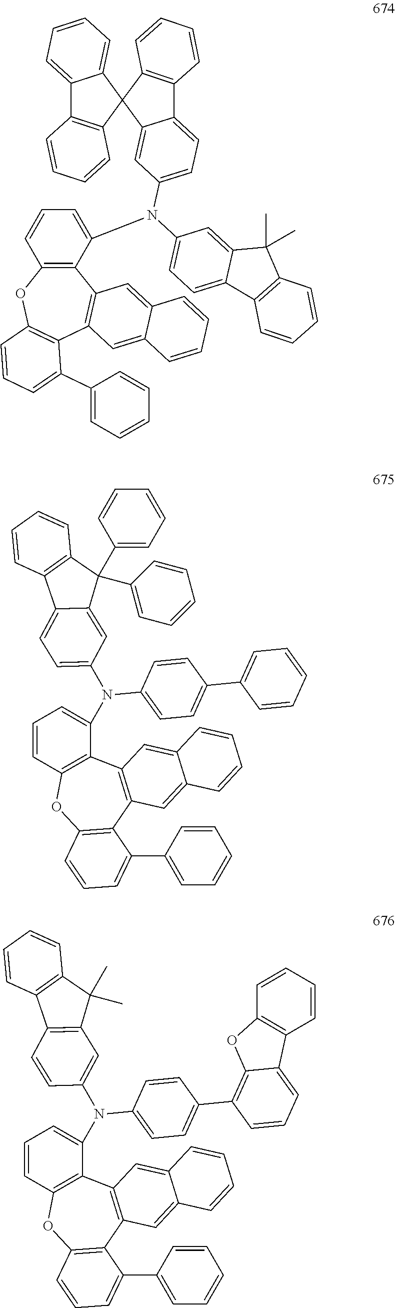 [SMILES: CC1(C)c2ccccc2-c2ccc(N(c3ccc4c(c3)C3(c5ccccc5-c5ccccc53)c3ccccc3-4)c3cccc4c3-c3cc5ccccc5cc3-c3c(cccc3-c3ccccc3)O4)cc21 ; c1ccc(-c2ccc(N(c3ccc4c(c3)C(c3ccccc3)(c3ccccc3)c3ccccc3-4)c3cccc4c3-c3cc5ccccc5cc3-c3c(cccc3-c3ccccc3)O4)cc2)cc1 ; CC1(C)c2ccccc2-c2ccc(N(c3ccc(-c4cccc5c4oc4ccccc45)cc3)c3cccc4c3-c3cc5ccccc5cc3-c3c(cccc3-c3ccccc3)O4)cc21]

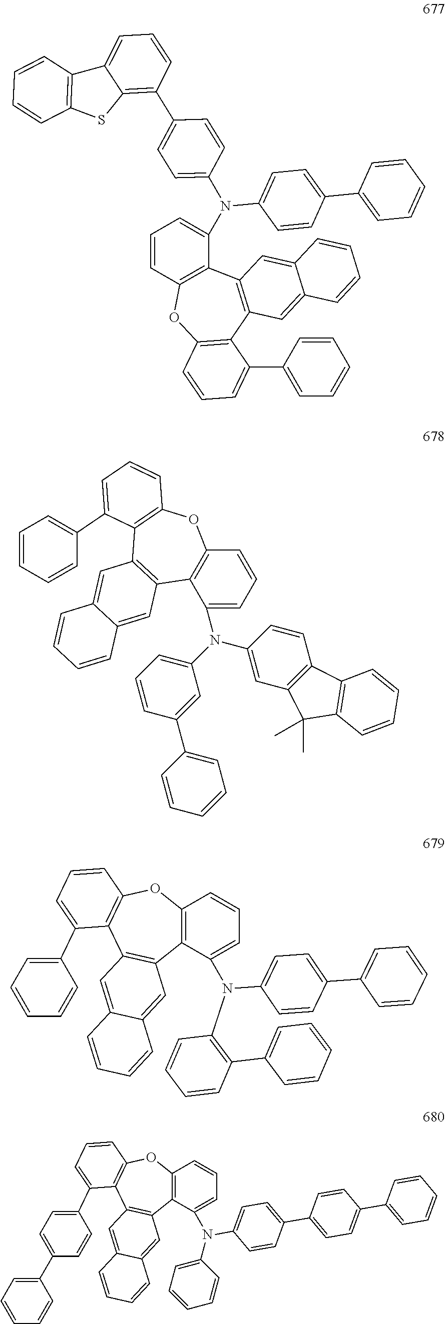 [SMILES: c1ccc(-c2ccc(N(c3ccc(-c4cccc5c4sc4ccccc45)cc3)c3cccc4c3-c3cc5ccccc5cc3-c3c(cccc3-c3ccccc3)O4)cc2)cc1 ; CC1(C)c2ccccc2-c2ccc(N(c3cccc(-c4ccccc4)c3)c3cccc4c3-c3cc5ccccc5cc3-c3c(cccc3-c3ccccc3)O4)cc21 ; c1ccc(-c2ccc(N(c3ccccc3-c3ccccc3)c3cccc4c3-c3cc5ccccc5cc3-c3c(cccc3-c3ccccc3)O4)cc2)cc1 ; c1ccc(-c2ccc(-c3ccc(N(c4ccccc4)c4cccc5c4-c4cc6ccccc6cc4-c4c(cccc4-c4ccc(-c6ccccc6)cc4)O5)cc3)cc2)cc1]

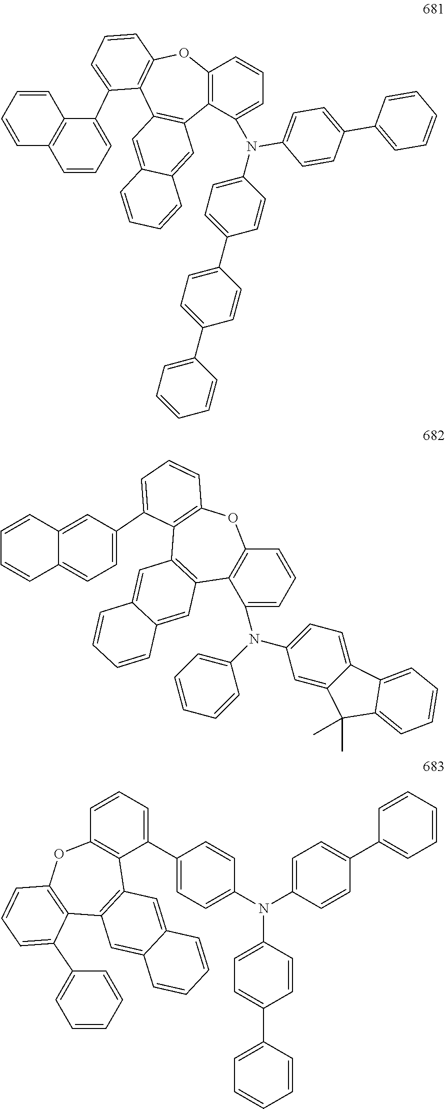

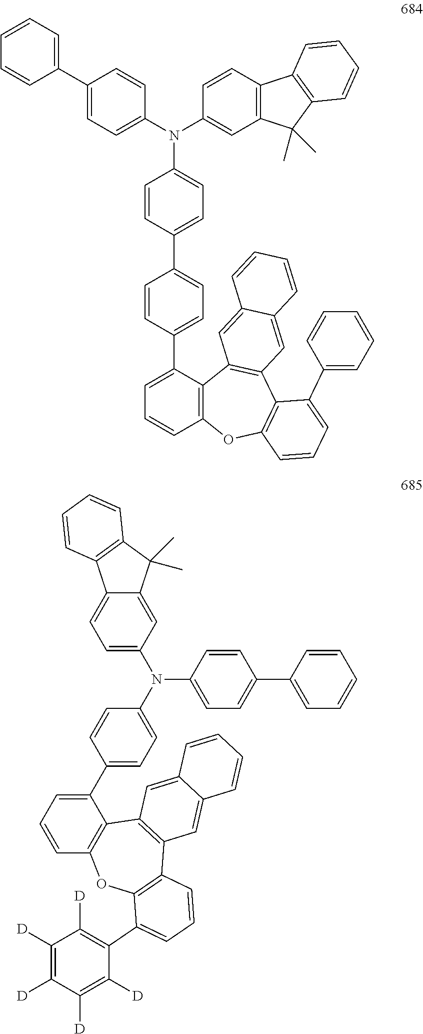 [SMILES: CC1(C)c2ccccc2-c2ccc(N(c3ccc(-c4ccccc4)cc3)c3ccc(-c4ccc(-c5cccc6c5-c5cc7ccccc7cc5-c5c(cccc5-c5ccccc5)O6)cc4)cc3)cc21 ; [2H]c1c([2H])c([2H])c(-c2cccc3c2Oc2cccc(-c4ccc(N(c5ccc(-c6ccccc6)cc5)c5ccc6c(c5)C(C)(C)c5ccccc5-6)cc4)c2-c2cc4ccccc4cc2-3)c([2H])c1[2H]]

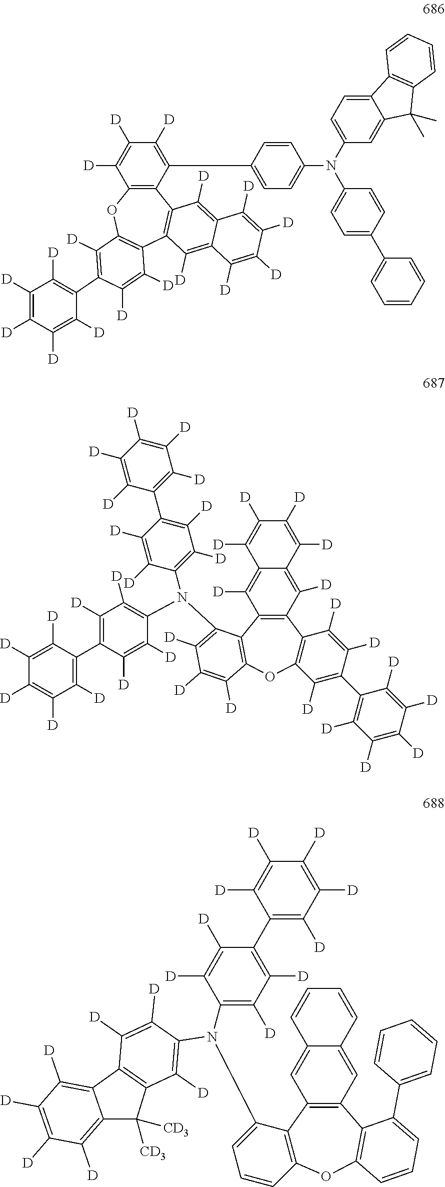

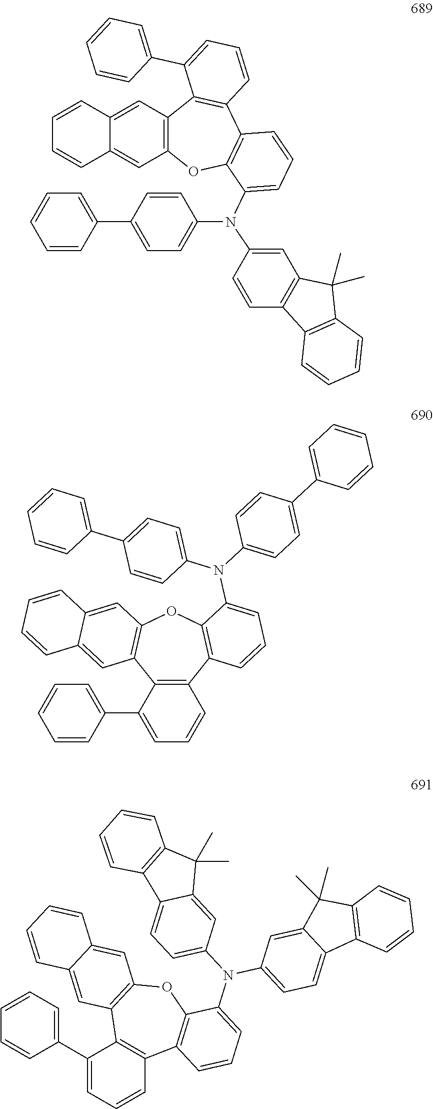

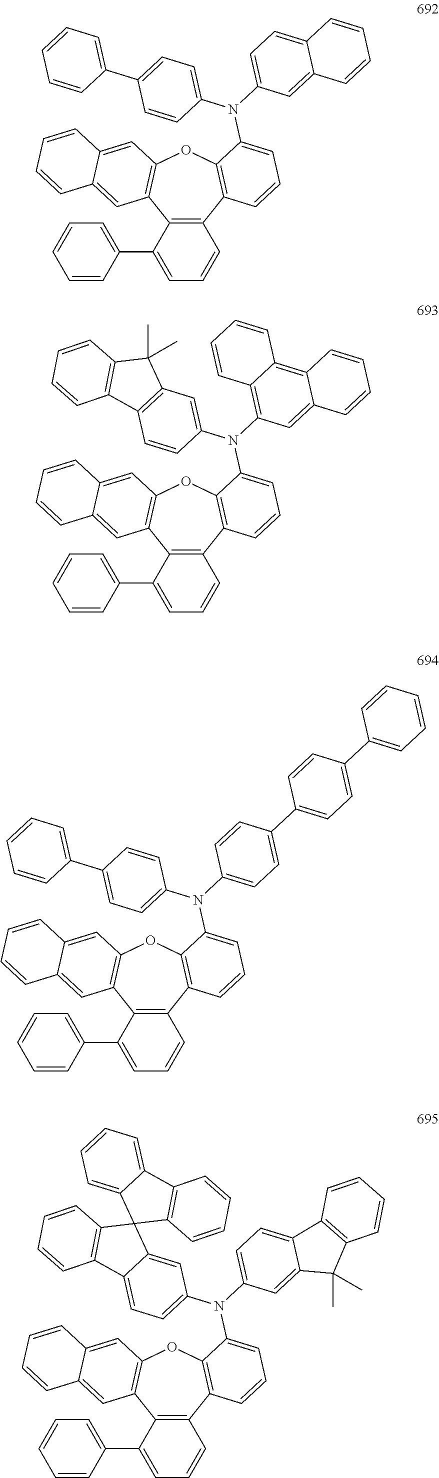 [SMILES: c1ccc(-c2ccc(N(c3ccc4ccccc4c3)c3cccc4c3Oc3cc5ccccc5cc3-c3c(-c5ccccc5)cccc3-4)cc2)cc1 ; CC1(C)c2ccccc2-c2ccc(N(c3cccc4c3Oc3cc5ccccc5cc3-c3c(-c5ccccc5)cccc3-4)c3cc4ccccc4c4ccccc34)cc21 ; c1ccc(-c2ccc(-c3ccc(N(c4ccc(-c5ccccc5)cc4)c4cccc5c4Oc4cc6ccccc6cc4-c4c(-c6ccccc6)cccc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cc5ccccc5cc3-c3c(-c5ccccc5)cccc3-4)cc21]

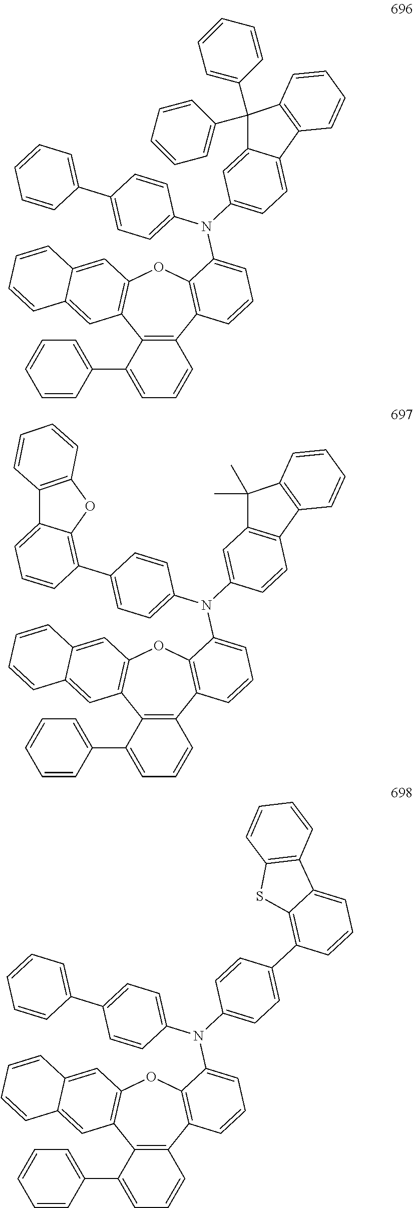

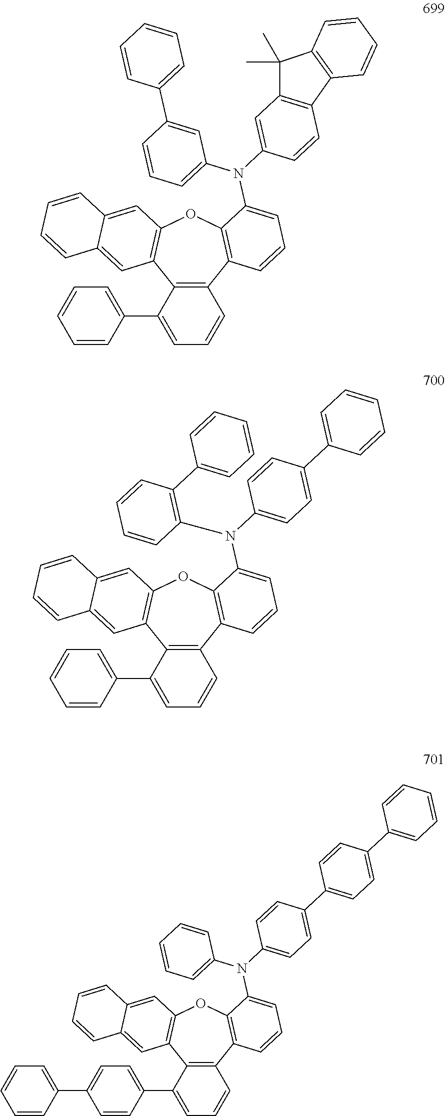 [SMILES: CC1(C)c2ccccc2-c2ccc(N(c3cccc(-c4ccccc4)c3)c3cccc4c3Oc3cc5ccccc5cc3-c3c(-c5ccccc5)cccc3-4)cc21 ; c1ccc(-c2ccc(N(c3ccccc3-c3ccccc3)c3cccc4c3Oc3cc5ccccc5cc3-c3c(-c5ccccc5)cccc3-4)cc2)cc1 ; c1ccc(-c2ccc(-c3ccc(N(c4ccccc4)c4cccc5c4Oc4cc6ccccc6cc4-c4c(-c6ccc(-c7ccccc7)cc6)cccc4-5)cc3)cc2)cc1]

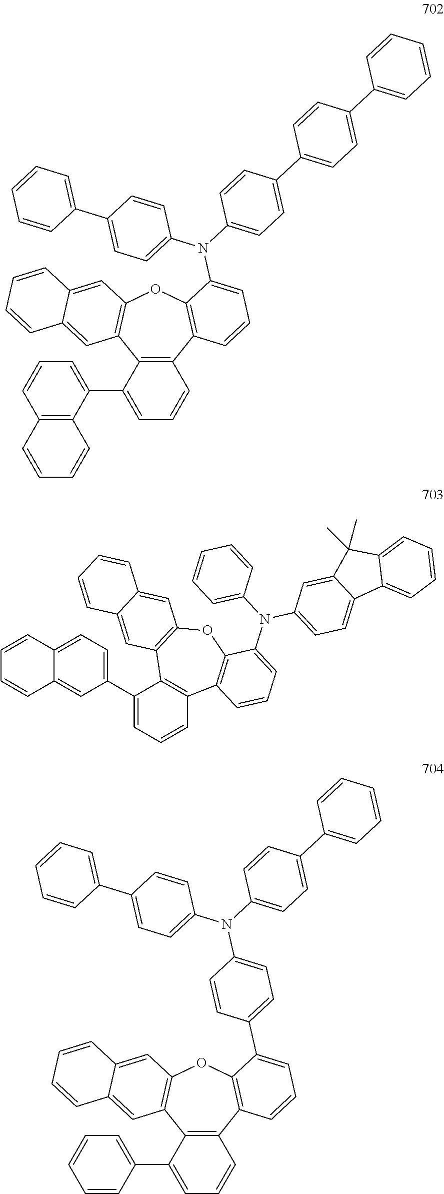 [SMILES: c1ccc(-c2ccc(-c3ccc(N(c4ccc(-c5ccccc5)cc4)c4cccc5c4Oc4cc6ccccc6cc4-c4c-5cccc4-c4cccc5ccccc45)cc3)cc2)cc1 ; CC1(C)c2ccccc2-c2ccc(N(c3ccccc3)c3cccc4c3Oc3cc5ccccc5cc3-c3c(-c5ccc6ccccc6c5)cccc3-4)cc21 ; c1ccc(-c2ccc(N(c3ccc(-c4ccccc4)cc3)c3ccc(-c4cccc5c4Oc4cc6ccccc6cc4-c4c(-c6ccccc6)cccc4-5)cc3)cc2)cc1]

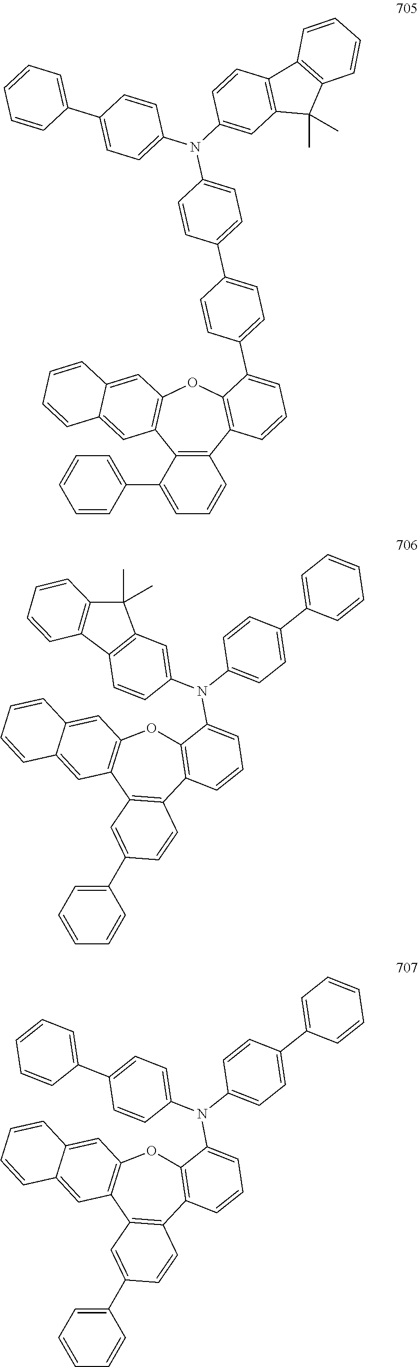 [SMILES: CC1(C)c2ccccc2-c2ccc(N(c3ccc(-c4ccccc4)cc3)c3ccc(-c4ccc(-c5cccc6c5Oc5cc7ccccc7cc5-c5c(-c7ccccc7)cccc5-6)cc4)cc3)cc21 ; CC1(C)c2ccccc2-c2ccc(N(c3ccc(-c4ccccc4)cc3)c3cccc4c3Oc3cc5ccccc5cc3-c3cc(-c5ccccc5)ccc3-4)cc21 ; c1ccc(-c2ccc(N(c3ccc(-c4ccccc4)cc3)c3cccc4c3Oc3cc5ccccc5cc3-c3cc(-c5ccccc5)ccc3-4)cc2)cc1]

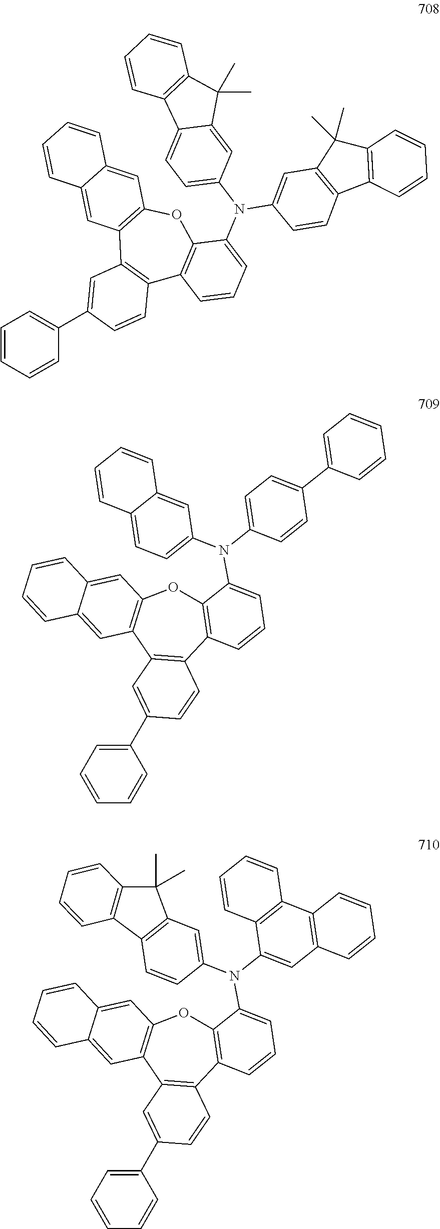 [SMILES: CC1(C)c2ccccc2-c2ccc(N(c3ccc4c(c3)C(C)(C)c3ccccc3-4)c3cccc4c3Oc3cc5ccccc5cc3-c3cc(-c5ccccc5)ccc3-4)cc21 ; c1ccc(-c2ccc(N(c3ccc4ccccc4c3)c3cccc4c3Oc3cc5ccccc5cc3-c3cc(-c5ccccc5)ccc3-4)cc2)cc1 ; CC1(C)c2ccccc2-c2ccc(N(c3cccc4c3Oc3cc5ccccc5cc3-c3cc(-c5ccccc5)ccc3-4)c3cc4ccccc4c4ccccc34)cc21]

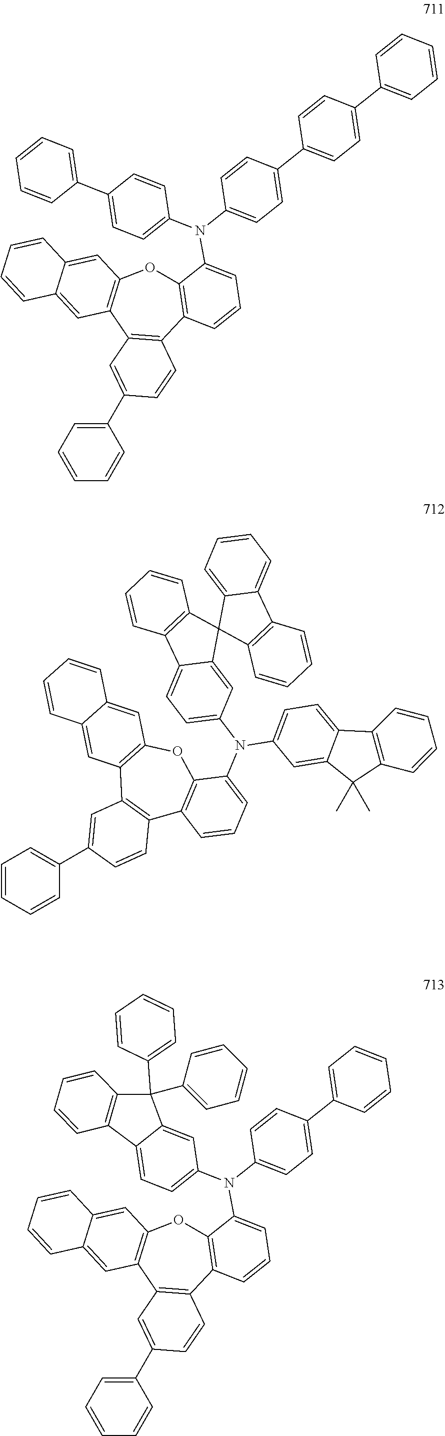 [SMILES: c1ccc(-c2ccc(-c3ccc(N(c4ccc(-c5ccccc5)cc4)c4cccc5c4Oc4cc6ccccc6cc4-c4cc(-c6ccccc6)ccc4-5)cc3)cc2)cc1 ; CC1(C)c2ccccc2-c2ccc(N(c3ccc4c(c3)C3(c5ccccc5-c5ccccc53)c3ccccc3-4)c3cccc4c3Oc3cc5ccccc5cc3-c3cc(-c5ccccc5)ccc3-4)cc21 ; c1ccc(-c2ccc(N(c3ccc4c(c3)C(c3ccccc3)(c3ccccc3)c3ccccc3-4)c3cccc4c3Oc3cc5ccccc5cc3-c3cc(-c5ccccc5)ccc3-4)cc2)cc1]

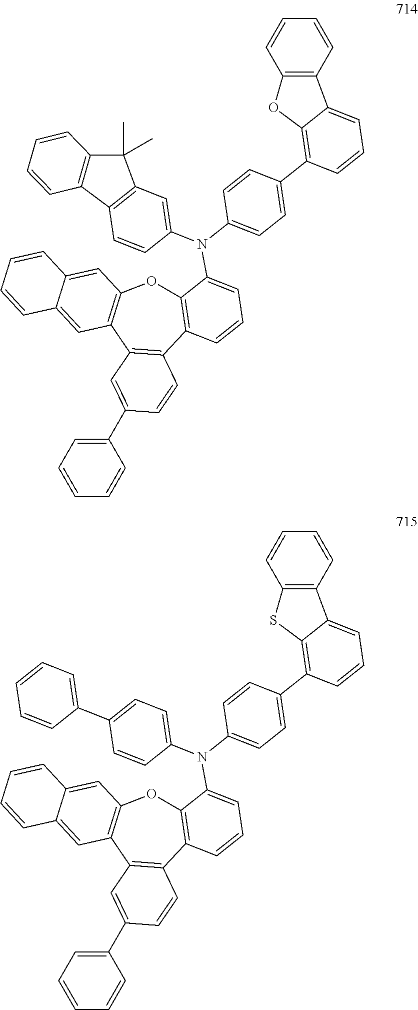 [SMILES: CC1(C)c2ccccc2-c2ccc(N(c3ccc(-c4cccc5c4oc4ccccc45)cc3)c3cccc4c3Oc3cc5ccccc5cc3-c3cc(-c5ccccc5)ccc3-4)cc21 ; c1ccc(-c2ccc(N(c3ccc(-c4cccc5c4sc4ccccc45)cc3)c3cccc4c3Oc3cc5ccccc5cc3-c3cc(-c5ccccc5)ccc3-4)cc2)cc1]

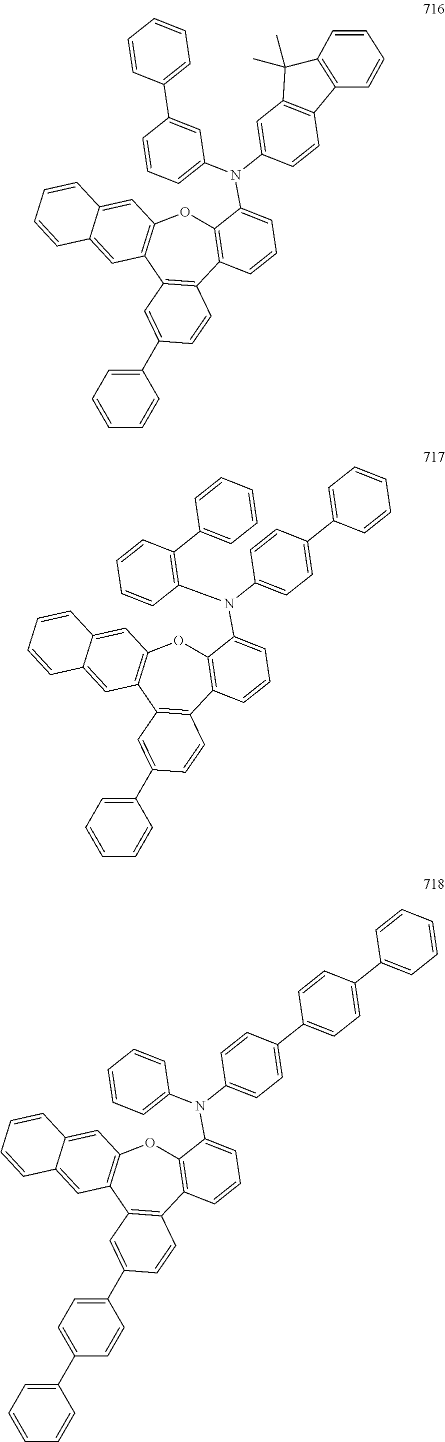 [SMILES: CC1(C)c2ccccc2-c2ccc(N(c3cccc(-c4ccccc4)c3)c3cccc4c3Oc3cc5ccccc5cc3-c3cc(-c5ccccc5)ccc3-4)cc21 ; c1ccc(-c2ccc(N(c3ccccc3-c3ccccc3)c3cccc4c3Oc3cc5ccccc5cc3-c3cc(-c5ccccc5)ccc3-4)cc2)cc1 ; c1ccc(-c2ccc(-c3ccc(N(c4ccccc4)c4cccc5c4Oc4cc6ccccc6cc4-c4cc(-c6ccc(-c7ccccc7)cc6)ccc4-5)cc3)cc2)cc1]

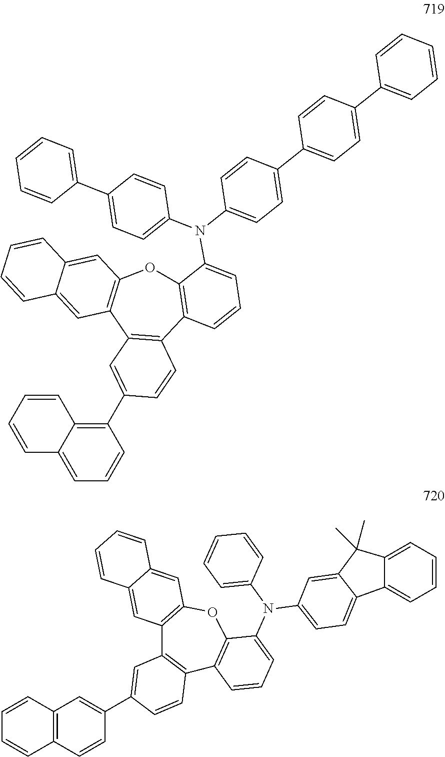 [SMILES: c1ccc(-c2ccc(-c3ccc(N(c4ccc(-c5ccccc5)cc4)c4cccc5c4Oc4cc6ccccc6cc4-c4cc(-c6cccc7ccccc67)ccc4-5)cc3)cc2)cc1 ; CC1(C)c2ccccc2-c2ccc(N(c3ccccc3)c3cccc4c3Oc3cc5ccccc5cc3-c3cc(-c5ccc6ccccc6c5)ccc3-4)cc21]

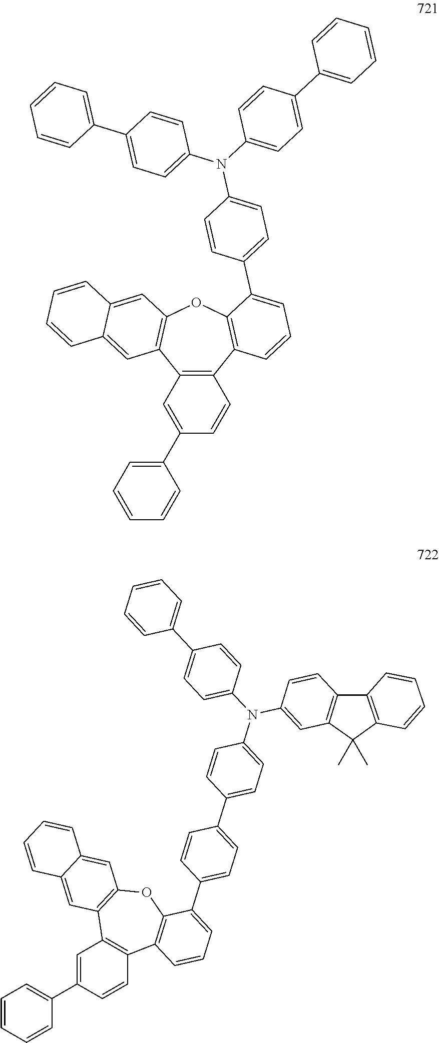 [SMILES: c1ccc(-c2ccc(N(c3ccc(-c4ccccc4)cc3)c3ccc(-c4cccc5c4Oc4cc6ccccc6cc4-c4cc(-c6ccccc6)ccc4-5)cc3)cc2)cc1 ; CC1(C)c2ccccc2-c2ccc(N(c3ccc(-c4ccccc4)cc3)c3ccc(-c4ccc(-c5cccc6c5Oc5cc7ccccc7cc5-c5cc(-c7ccccc7)ccc5-6)cc4)cc3)cc21]

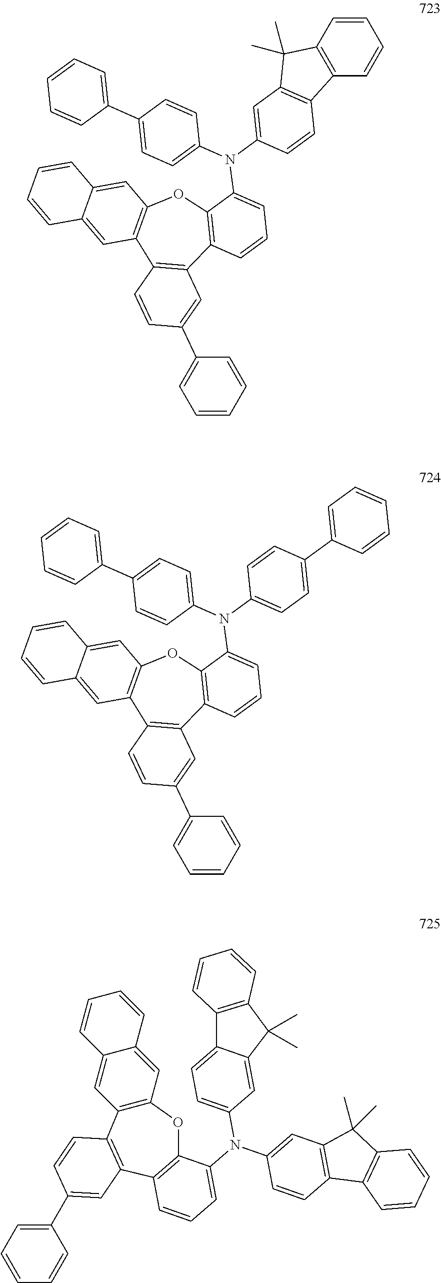 [SMILES: CC1(C)c2ccccc2-c2ccc(N(c3ccc(-c4ccccc4)cc3)c3cccc4c3Oc3cc5ccccc5cc3-c3ccc(-c5ccccc5)cc3-4)cc21 ; c1ccc(-c2ccc(N(c3ccc(-c4ccccc4)cc3)c3cccc4c3Oc3cc5ccccc5cc3-c3ccc(-c5ccccc5)cc3-4)cc2)cc1 ; CC1(C)c2ccccc2-c2ccc(N(c3ccc4c(c3)C(C)(C)c3ccccc3-4)c3cccc4c3Oc3cc5ccccc5cc3-c3ccc(-c5ccccc5)cc3-4)cc21]

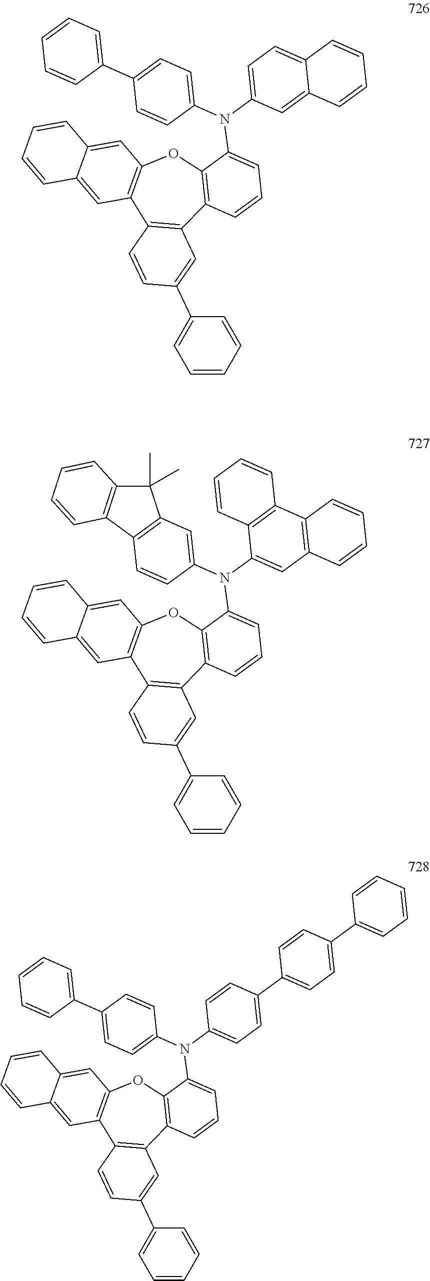 [SMILES: c1ccc(-c2ccc(N(c3ccc4ccccc4c3)c3cccc4c3Oc3cc5ccccc5cc3-c3ccc(-c5ccccc5)cc3-4)cc2)cc1 ; CC1(C)c2ccccc2-c2ccc(N(c3cccc4c3Oc3cc5ccccc5cc3-c3ccc(-c5ccccc5)cc3-4)c3cc4ccccc4c4ccccc34)cc21 ; c1ccc(-c2ccc(-c3ccc(N(c4ccc(-c5ccccc5)cc4)c4cccc5c4Oc4cc6ccccc6cc4-c4ccc(-c6ccccc6)cc4-5)cc3)cc2)cc1]

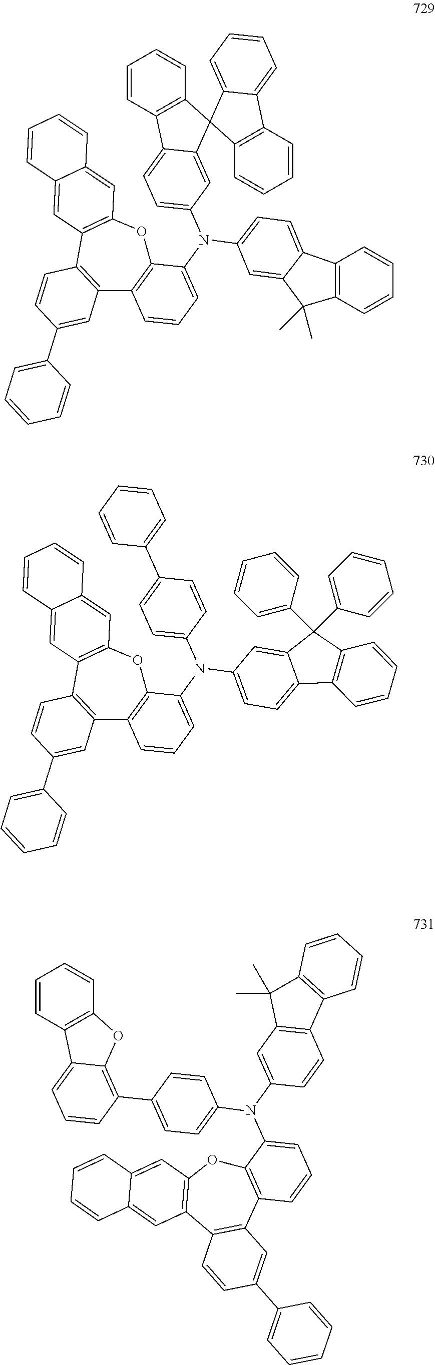 [SMILES: CC1(C)c2ccccc2-c2ccc(N(c3ccc4c(c3)C3(c5ccccc5-c5ccccc53)c3ccccc3-4)c3cccc4c3Oc3cc5ccccc5cc3-c3ccc(-c5ccccc5)cc3-4)cc21 ; c1ccc(-c2ccc(N(c3ccc4c(c3)C(c3ccccc3)(c3ccccc3)c3ccccc3-4)c3cccc4c3Oc3cc5ccccc5cc3-c3ccc(-c5ccccc5)cc3-4)cc2)cc1 ; CC1(C)c2ccccc2-c2ccc(N(c3ccc(-c4cccc5c4oc4ccccc45)cc3)c3cccc4c3Oc3cc5ccccc5cc3-c3ccc(-c5ccccc5)cc3-4)cc21]

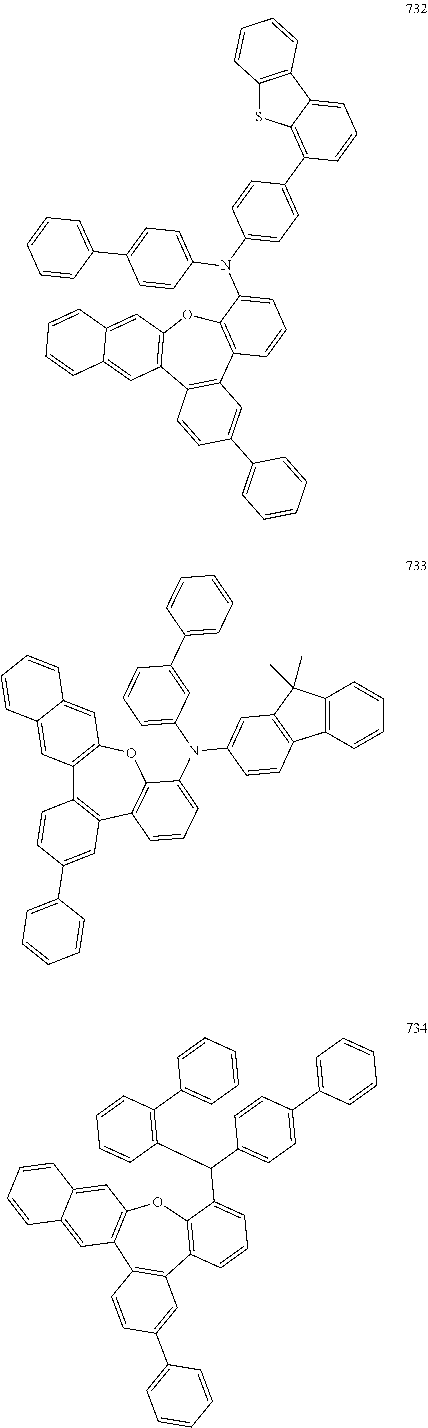 [SMILES: c1ccc(-c2ccc(N(c3ccc(-c4cccc5c4sc4ccccc45)cc3)c3cccc4c3Oc3cc5ccccc5cc3-c3ccc(-c5ccccc5)cc3-4)cc2)cc1 ; CC1(C)c2ccccc2-c2ccc(N(c3cccc(-c4ccccc4)c3)c3cccc4c3Oc3cc5ccccc5cc3-c3ccc(-c5ccccc5)cc3-4)cc21 ; c1ccc(-c2ccc(C(c3ccccc3-c3ccccc3)c3cccc4c3Oc3cc5ccccc5cc3-c3ccc(-c5ccccc5)cc3-4)cc2)cc1]

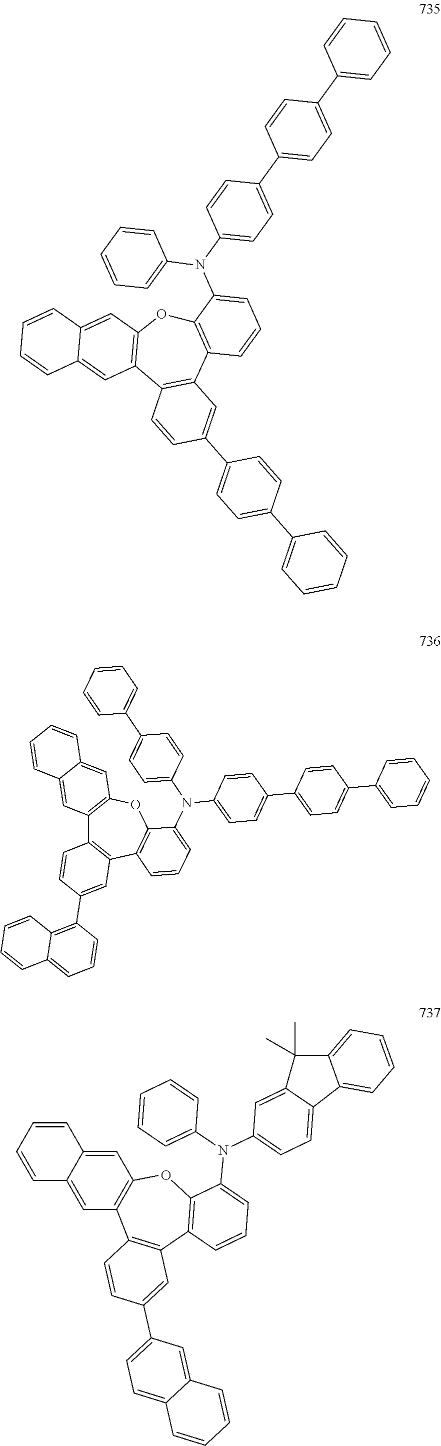 [SMILES: c1ccc(-c2ccc(-c3ccc(N(c4ccccc4)c4cccc5c4Oc4cc6ccccc6cc4-c4ccc(-c6ccc(-c7ccccc7)cc6)cc4-5)cc3)cc2)cc1 ; c1ccc(-c2ccc(-c3ccc(N(c4ccc(-c5ccccc5)cc4)c4cccc5c4Oc4cc6ccccc6cc4-c4ccc(-c6cccc7ccccc67)cc4-5)cc3)cc2)cc1 ; CC1(C)c2ccccc2-c2ccc(N(c3ccccc3)c3cccc4c3Oc3cc5ccccc5cc3-c3ccc(-c5ccc6ccccc6c5)cc3-4)cc21]

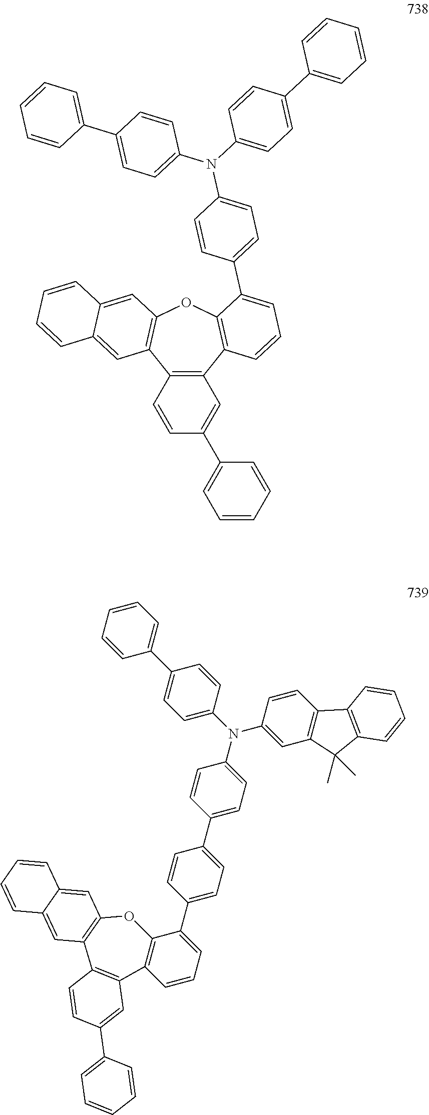 [SMILES: c1ccc(-c2ccc(N(c3ccc(-c4ccccc4)cc3)c3ccc(-c4cccc5c4Oc4cc6ccccc6cc4-c4ccc(-c6ccccc6)cc4-5)cc3)cc2)cc1 ; CC1(C)c2ccccc2-c2ccc(N(c3ccc(-c4ccccc4)cc3)c3ccc(-c4ccc(-c5cccc6c5Oc5cc7ccccc7cc5-c5ccc(-c7ccccc7)cc5-6)cc4)cc3)cc21]

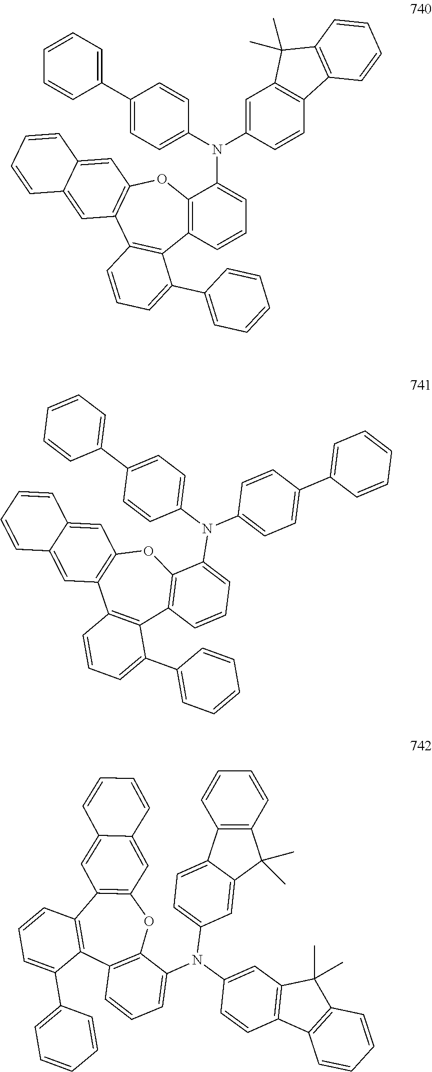 [SMILES: CC1(C)c2ccccc2-c2ccc(N(c3ccc(-c4ccccc4)cc3)c3cccc4c3Oc3cc5ccccc5cc3-c3cccc(-c5ccccc5)c3-4)cc21 ; c1ccc(-c2ccc(N(c3ccc(-c4ccccc4)cc3)c3cccc4c3Oc3cc5ccccc5cc3-c3cccc(-c5ccccc5)c3-4)cc2)cc1 ; CC1(C)c2ccccc2-c2ccc(N(c3ccc4c(c3)C(C)(C)c3ccccc3-4)c3cccc4c3Oc3cc5ccccc5cc3-c3cccc(-c5ccccc5)c3-4)cc21]

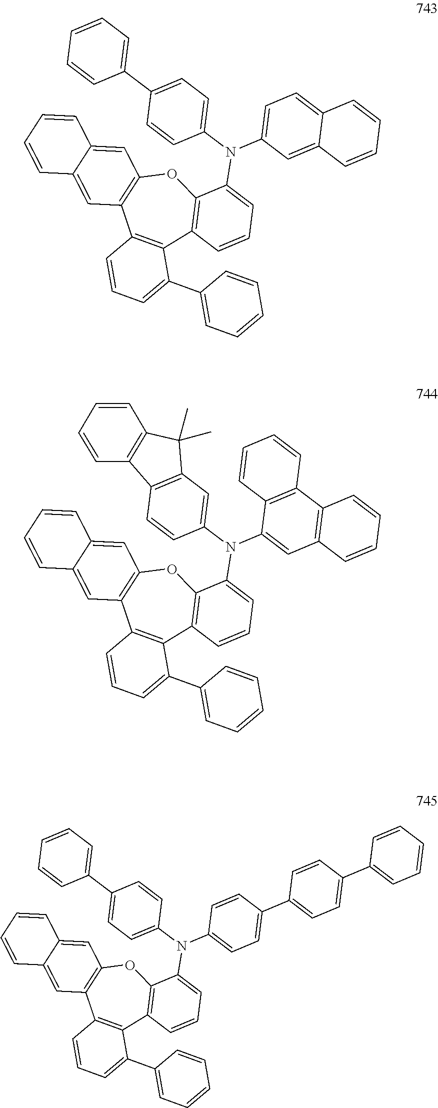 [SMILES: c1ccc(-c2ccc(N(c3ccc4ccccc4c3)c3cccc4c3Oc3cc5ccccc5cc3-c3cccc(-c5ccccc5)c3-4)cc2)cc1 ; CC1(C)c2ccccc2-c2ccc(N(c3cccc4c3Oc3cc5ccccc5cc3-c3cccc(-c5ccccc5)c3-4)c3cc4ccccc4c4ccccc34)cc21 ; c1ccc(-c2ccc(-c3ccc(N(c4ccc(-c5ccccc5)cc4)c4cccc5c4Oc4cc6ccccc6cc4-c4cccc(-c6ccccc6)c4-5)cc3)cc2)cc1]

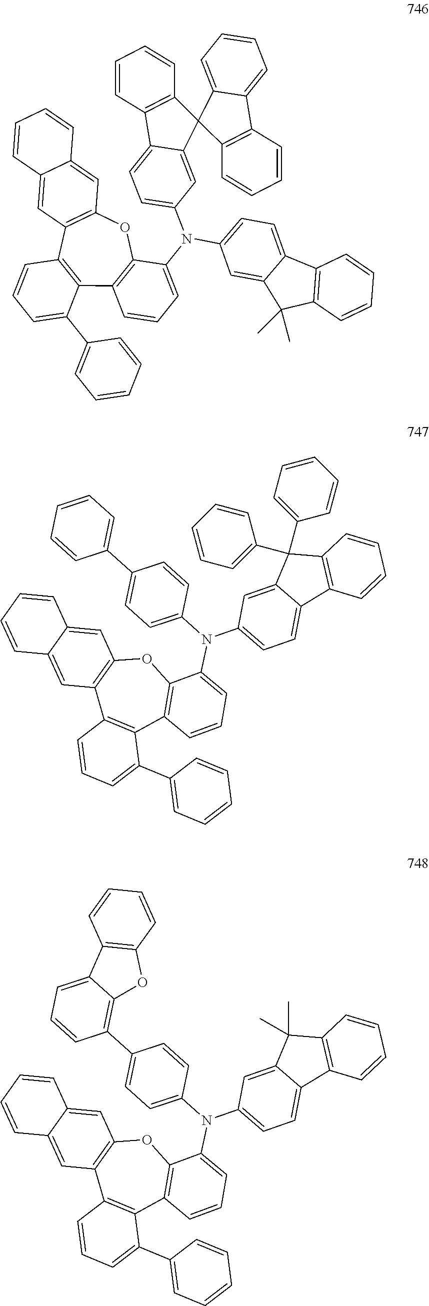 [SMILES: CC1(C)c2ccccc2-c2ccc(N(c3ccc4c(c3)C3(c5ccccc5-c5ccccc53)c3ccccc3-4)c3cccc4c3Oc3cc5ccccc5cc3-c3cccc(-c5ccccc5)c3-4)cc21 ; c1ccc(-c2ccc(N(c3ccc4c(c3)C(c3ccccc3)(c3ccccc3)c3ccccc3-4)c3cccc4c3Oc3cc5ccccc5cc3-c3cccc(-c5ccccc5)c3-4)cc2)cc1 ; CC1(C)c2ccccc2-c2ccc(N(c3ccc(-c4cccc5c4oc4ccccc45)cc3)c3cccc4c3Oc3cc5ccccc5cc3-c3cccc(-c5ccccc5)c3-4)cc21]

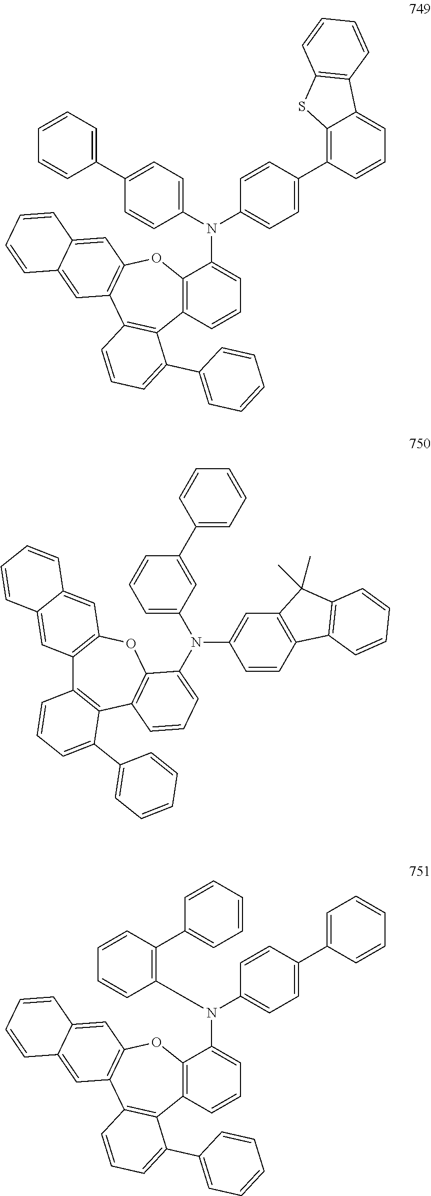 [SMILES: c1ccc(-c2ccc(N(c3ccc(-c4cccc5c4sc4ccccc45)cc3)c3cccc4c3Oc3cc5ccccc5cc3-c3cccc(-c5ccccc5)c3-4)cc2)cc1 ; CC1(C)c2ccccc2-c2ccc(N(c3cccc(-c4ccccc4)c3)c3cccc4c3Oc3cc5ccccc5cc3-c3cccc(-c5ccccc5)c3-4)cc21 ; c1ccc(-c2ccc(N(c3ccccc3-c3ccccc3)c3cccc4c3Oc3cc5ccccc5cc3-c3cccc(-c5ccccc5)c3-4)cc2)cc1]

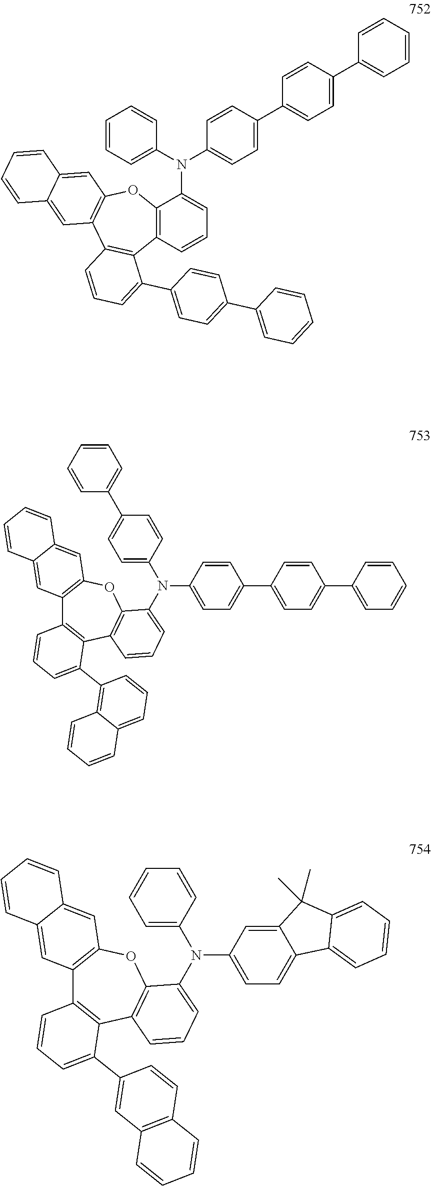 [SMILES: c1ccc(-c2ccc(-c3ccc(N(c4ccccc4)c4cccc5c4Oc4cc6ccccc6cc4-c4cccc(-c6ccc(-c7ccccc7)cc6)c4-5)cc3)cc2)cc1 ; c1ccc(-c2ccc(-c3ccc(N(c4ccc(-c5ccccc5)cc4)c4cccc5c4Oc4cc6ccccc6cc4-c4cccc(-c6cccc7ccccc67)c4-5)cc3)cc2)cc1 ; CC1(C)c2ccccc2-c2ccc(N(c3ccccc3)c3cccc4c3Oc3cc5ccccc5cc3-c3cccc(-c5ccc6ccccc6c5)c3-4)cc21]

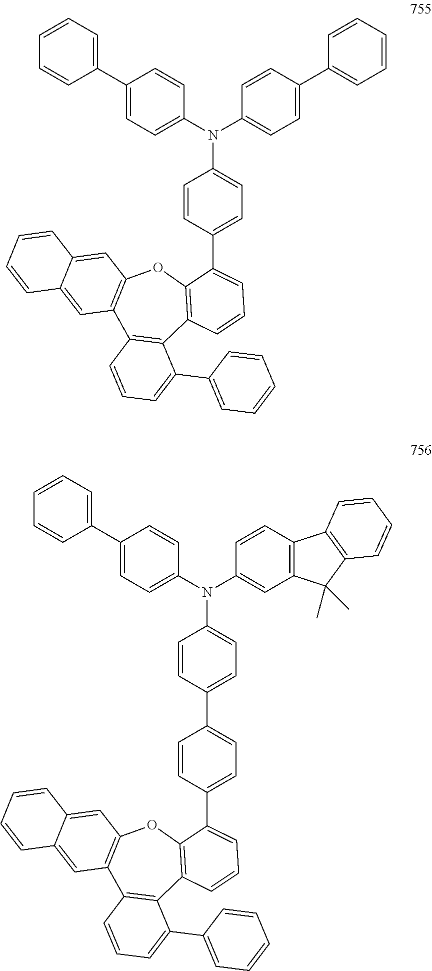 [SMILES: c1ccc(-c2ccc(N(c3ccc(-c4ccccc4)cc3)c3ccc(-c4cccc5c4Oc4cc6ccccc6cc4-c4cccc(-c6ccccc6)c4-5)cc3)cc2)cc1 ; CC1(C)c2ccccc2-c2ccc(N(c3ccc(-c4ccccc4)cc3)c3ccc(-c4ccc(-c5cccc6c5Oc5cc7ccccc7cc5-c5cccc(-c7ccccc7)c5-6)cc4)cc3)cc21]

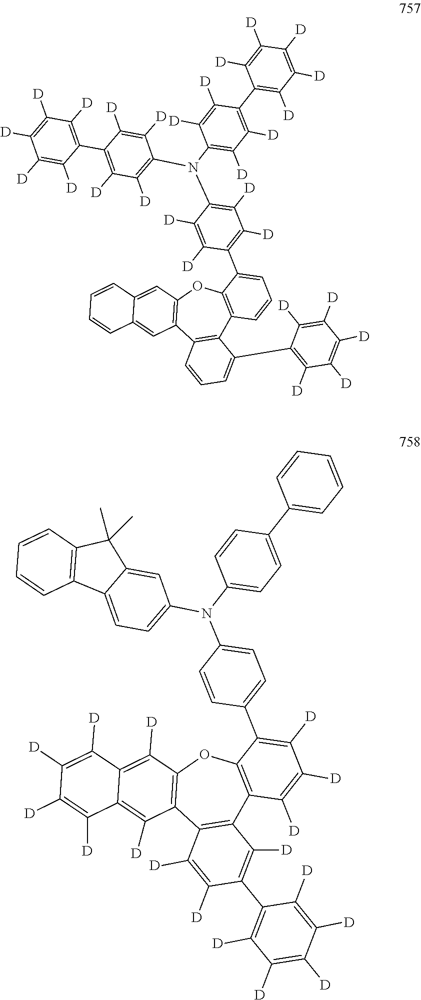 [SMILES: [2H]c1c([2H])c([2H])c(-c2cccc3c2-c2cccc(-c4c([2H])c([2H])c(N(c5c([2H])c([2H])c(-c6c([2H])c([2H])c([2H])c([2H])c6[2H])c([2H])c5[2H])c5c([2H])c([2H])c(-c6c([2H])c([2H])c([2H])c([2H])c6[2H])c([2H])c5[2H])c([2H])c4[2H])c2Oc2cc4ccccc4cc2-3)c([2H])c1[2H] ; [2H]c1c([2H])c([2H])c(-c2c([2H])c([2H])c3c(c2[2H])-c2c([2H])c([2H])c([2H])c(-c4ccc(N(c5ccc(-c6ccccc6)cc5)c5ccc6c(c5)C(C)(C)c5ccccc5-6)cc4)c2Oc2c-3c([2H])c3c([2H])c([2H])c([2H])c([2H])c3c2[2H])c([2H])c1[2H]]

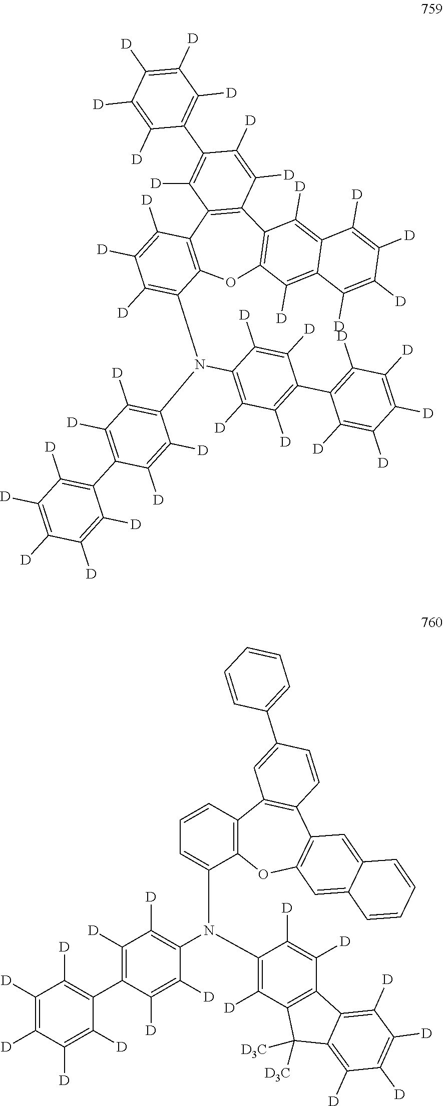

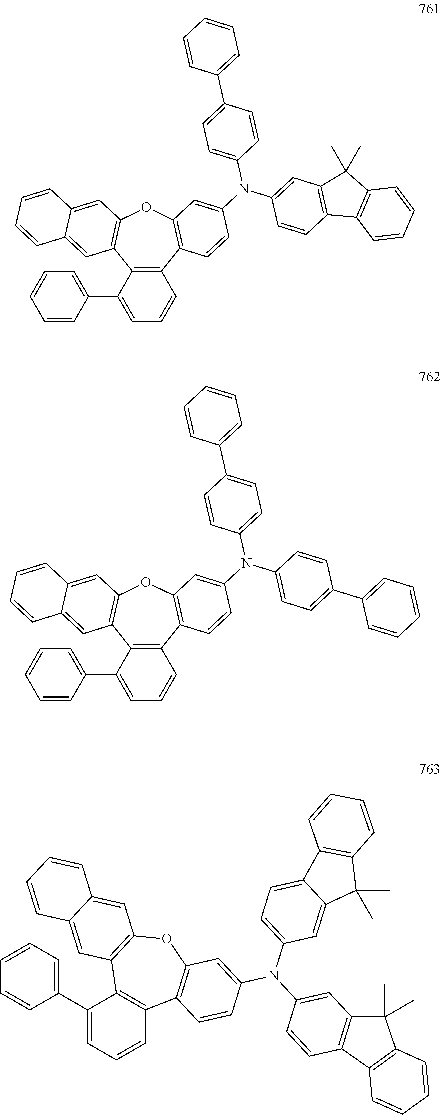 [SMILES: CC1(C)c2ccccc2-c2ccc(N(c3ccc(-c4ccccc4)cc3)c3ccc4c(c3)Oc3cc5ccccc5cc3-c3c(-c5ccccc5)cccc3-4)cc21 ; c1ccc(-c2ccc(N(c3ccc(-c4ccccc4)cc3)c3ccc4c(c3)Oc3cc5ccccc5cc3-c3c(-c5ccccc5)cccc3-4)cc2)cc1 ; CC1(C)c2ccccc2-c2ccc(N(c3ccc4c(c3)Oc3cc5ccccc5cc3-c3c(-c5ccccc5)cccc3-4)c3ccc4c(c3)C(C)(C)c3ccccc3-4)cc21]

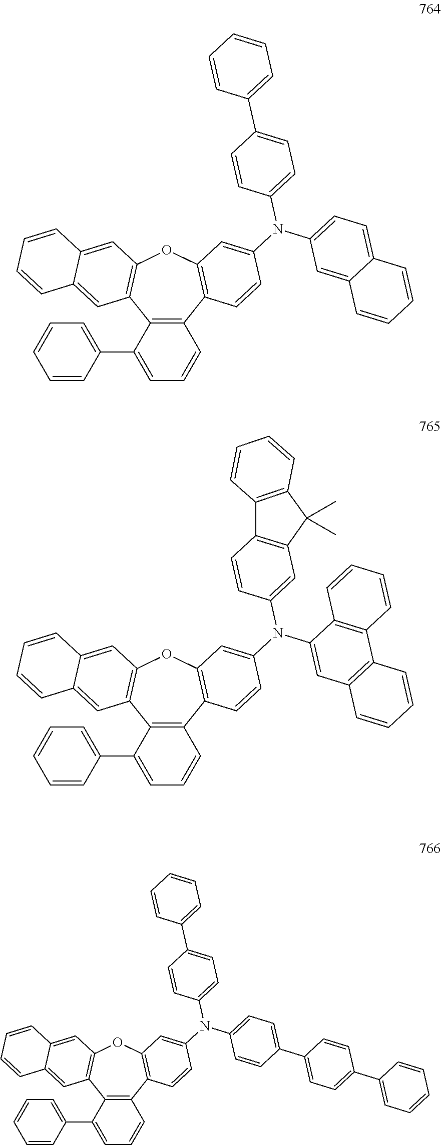 [SMILES: c1ccc(-c2ccc(N(c3ccc4c(c3)Oc3cc5ccccc5cc3-c3c(-c5ccccc5)cccc3-4)c3ccc4ccccc4c3)cc2)cc1 ; CC1(C)c2ccccc2-c2ccc(N(c3ccc4c(c3)Oc3cc5ccccc5cc3-c3c(-c5ccccc5)cccc3-4)c3cc4ccccc4c4ccccc34)cc21 ; c1ccc(-c2ccc(-c3ccc(N(c4ccc(-c5ccccc5)cc4)c4ccc5c(c4)Oc4cc6ccccc6cc4-c4c(-c6ccccc6)cccc4-5)cc3)cc2)cc1]

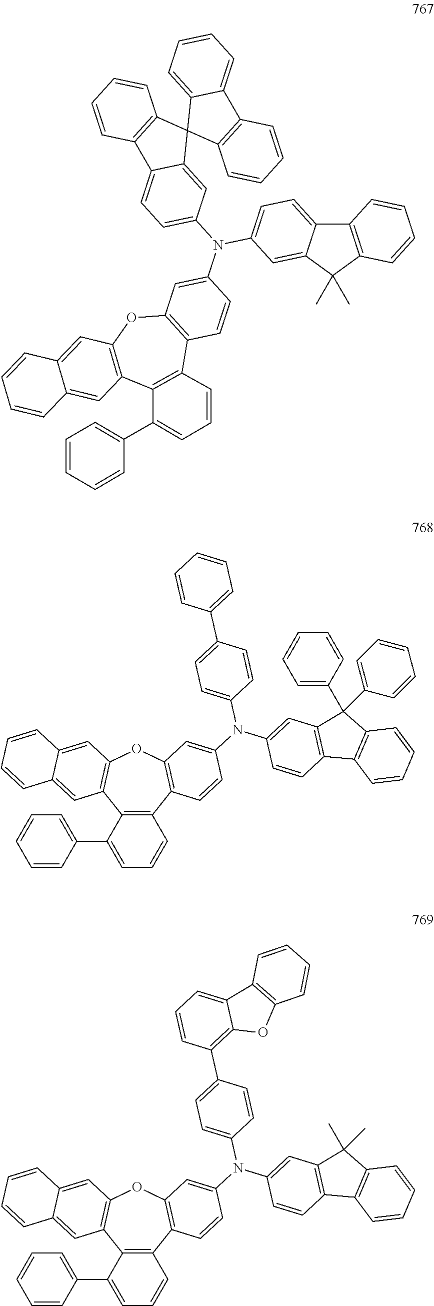 [SMILES: CC1(C)c2ccccc2-c2ccc(N(c3ccc4c(c3)Oc3cc5ccccc5cc3-c3c(-c5ccccc5)cccc3-4)c3ccc4c(c3)C3(c5ccccc5-c5ccccc53)c3ccccc3-4)cc21 ; c1ccc(-c2ccc(N(c3ccc4c(c3)Oc3cc5ccccc5cc3-c3c(-c5ccccc5)cccc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc5ccccc5cc3-c3c(-c5ccccc5)cccc3-4)cc21]

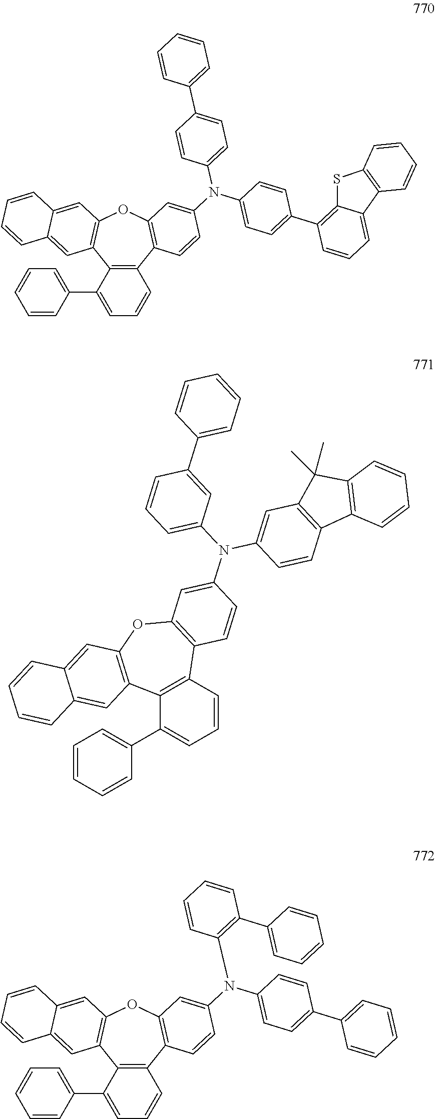 [SMILES: c1ccc(-c2ccc(N(c3ccc(-c4cccc5c4sc4ccccc45)cc3)c3ccc4c(c3)Oc3cc5ccccc5cc3-c3c(-c5ccccc5)cccc3-4)cc2)cc1 ; CC1(C)c2ccccc2-c2ccc(N(c3cccc(-c4ccccc4)c3)c3ccc4c(c3)Oc3cc5ccccc5cc3-c3c(-c5ccccc5)cccc3-4)cc21 ; c1ccc(-c2ccc(N(c3ccc4c(c3)Oc3cc5ccccc5cc3-c3c(-c5ccccc5)cccc3-4)c3ccccc3-c3ccccc3)cc2)cc1]

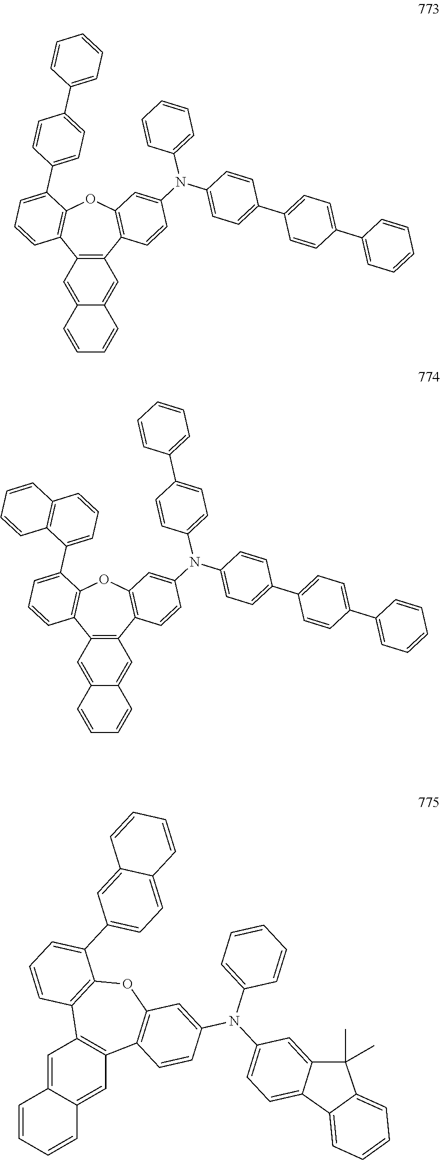 [SMILES: c1ccc(-c2ccc(-c3ccc(N(c4ccccc4)c4ccc5c(c4)Oc4c(-c6ccc(-c7ccccc7)cc6)cccc4-c4cc6ccccc6cc4-5)cc3)cc2)cc1 ; c1ccc(-c2ccc(-c3ccc(N(c4ccc(-c5ccccc5)cc4)c4ccc5c(c4)Oc4c(cccc4-c4cccc6ccccc46)-c4cc6ccccc6cc4-5)cc3)cc2)cc1 ; CC1(C)c2ccccc2-c2ccc(N(c3ccccc3)c3ccc4c(c3)Oc3c(-c5ccc6ccccc6c5)cccc3-c3cc5ccccc5cc3-4)cc21]

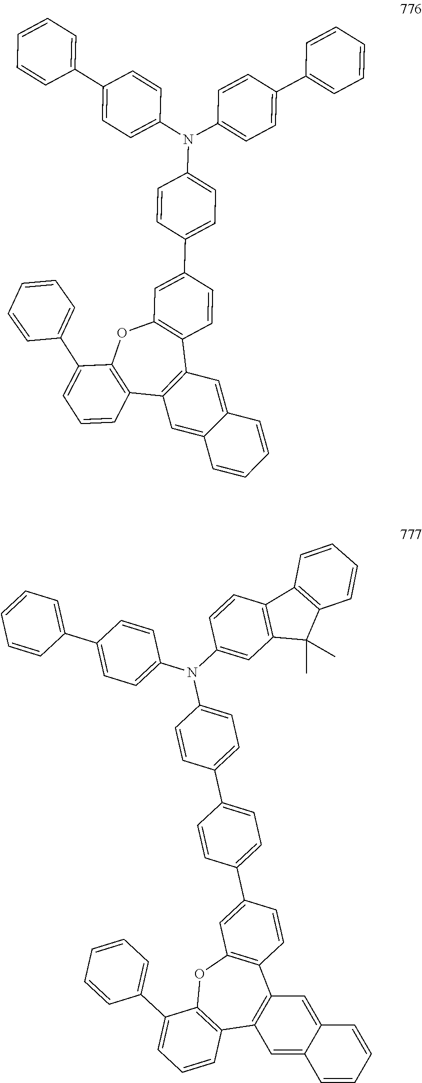 [SMILES: c1ccc(-c2ccc(N(c3ccc(-c4ccccc4)cc3)c3ccc(-c4ccc5c(c4)Oc4c(-c6ccccc6)cccc4-c4cc6ccccc6cc4-5)cc3)cc2)cc1 ; CC1(C)c2ccccc2-c2ccc(N(c3ccc(-c4ccccc4)cc3)c3ccc(-c4ccc(-c5ccc6c(c5)Oc5c(-c7ccccc7)cccc5-c5cc7ccccc7cc5-6)cc4)cc3)cc21]

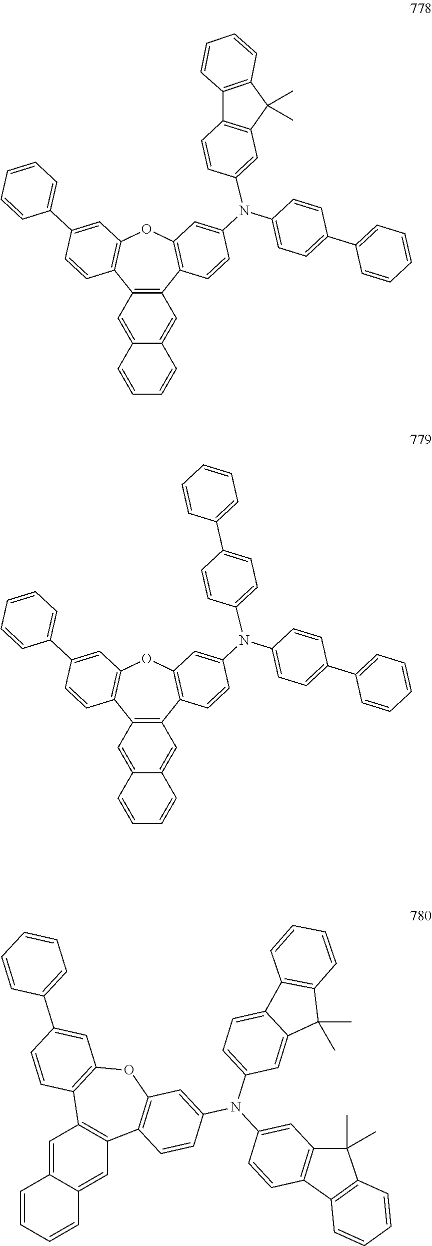 [SMILES: CC1(C)c2ccccc2-c2ccc(N(c3ccc(-c4ccccc4)cc3)c3ccc4c(c3)Oc3cc(-c5ccccc5)ccc3-c3cc5ccccc5cc3-4)cc21 ; c1ccc(-c2ccc(N(c3ccc(-c4ccccc4)cc3)c3ccc4c(c3)Oc3cc(-c5ccccc5)ccc3-c3cc5ccccc5cc3-4)cc2)cc1 ; CC1(C)c2ccccc2-c2ccc(N(c3ccc4c(c3)Oc3cc(-c5ccccc5)ccc3-c3cc5ccccc5cc3-4)c3ccc4c(c3)C(C)(C)c3ccccc3-4)cc21]

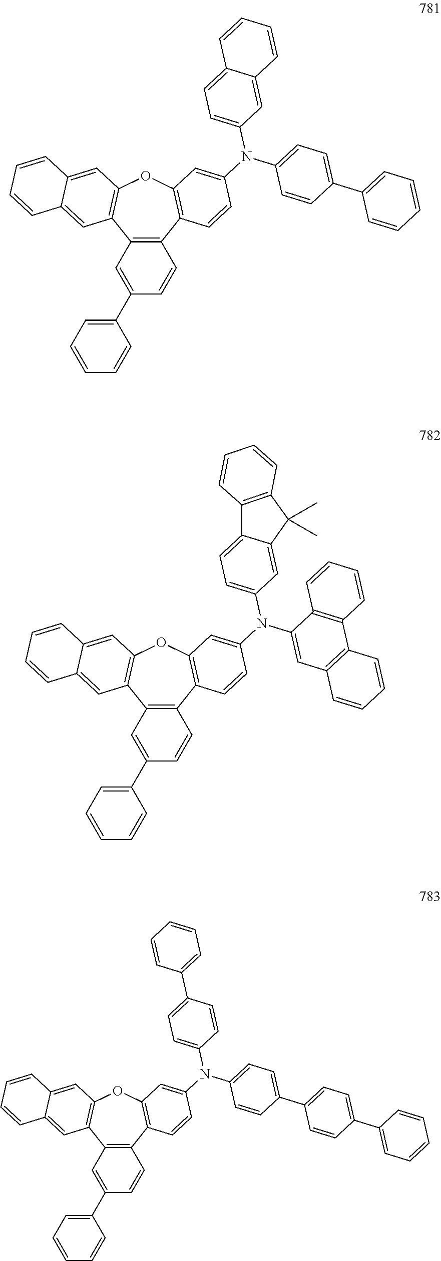 [SMILES: c1ccc(-c2ccc(N(c3ccc4c(c3)Oc3cc5ccccc5cc3-c3cc(-c5ccccc5)ccc3-4)c3ccc4ccccc4c3)cc2)cc1 ; CC1(C)c2ccccc2-c2ccc(N(c3ccc4c(c3)Oc3cc5ccccc5cc3-c3cc(-c5ccccc5)ccc3-4)c3cc4ccccc4c4ccccc34)cc21 ; c1ccc(-c2ccc(-c3ccc(N(c4ccc(-c5ccccc5)cc4)c4ccc5c(c4)Oc4cc6ccccc6cc4-c4cc(-c6ccccc6)ccc4-5)cc3)cc2)cc1]

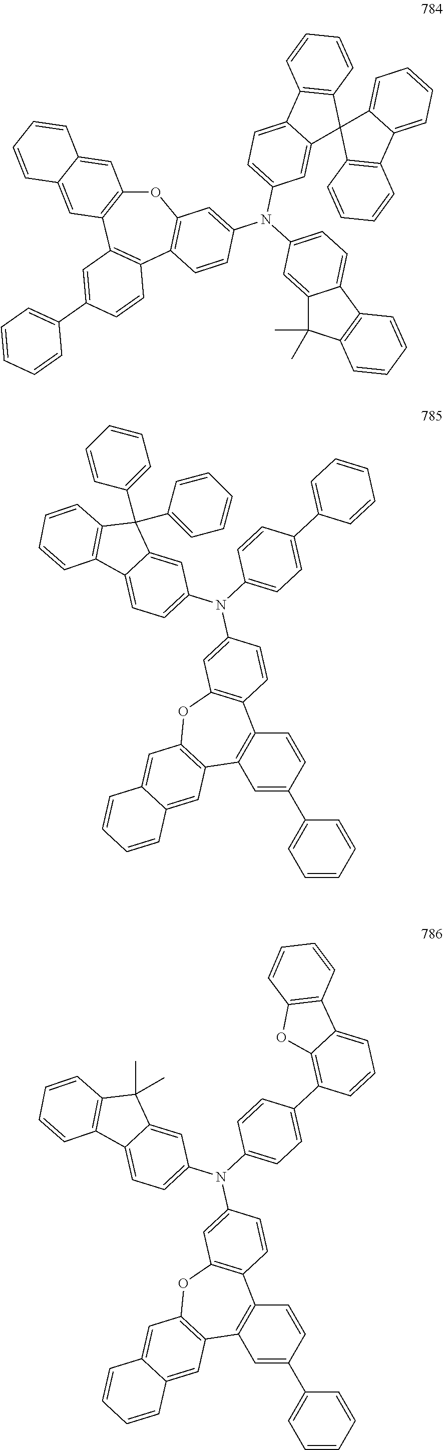 [SMILES: CC1(C)c2ccccc2-c2ccc(N(c3ccc4c(c3)Oc3cc5ccccc5cc3-c3cc(-c5ccccc5)ccc3-4)c3ccc4c(c3)C3(c5ccccc5-c5ccccc53)c3ccccc3-4)cc21 ; c1ccc(-c2ccc(N(c3ccc4c(c3)Oc3cc5ccccc5cc3-c3cc(-c5ccccc5)ccc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc5ccccc5cc3-c3cc(-c5ccccc5)ccc3-4)cc21]

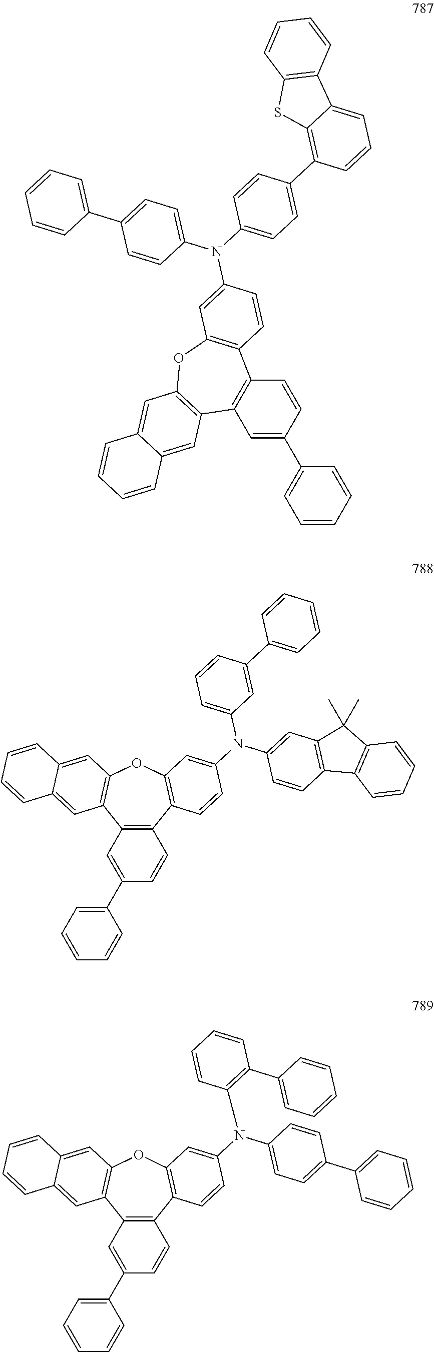 [SMILES: c1ccc(-c2ccc(N(c3ccc(-c4cccc5c4sc4ccccc45)cc3)c3ccc4c(c3)Oc3cc5ccccc5cc3-c3cc(-c5ccccc5)ccc3-4)cc2)cc1 ; CC1(C)c2ccccc2-c2ccc(N(c3cccc(-c4ccccc4)c3)c3ccc4c(c3)Oc3cc5ccccc5cc3-c3cc(-c5ccccc5)ccc3-4)cc21 ; c1ccc(-c2ccc(N(c3ccc4c(c3)Oc3cc5ccccc5cc3-c3cc(-c5ccccc5)ccc3-4)c3ccccc3-c3ccccc3)cc2)cc1]

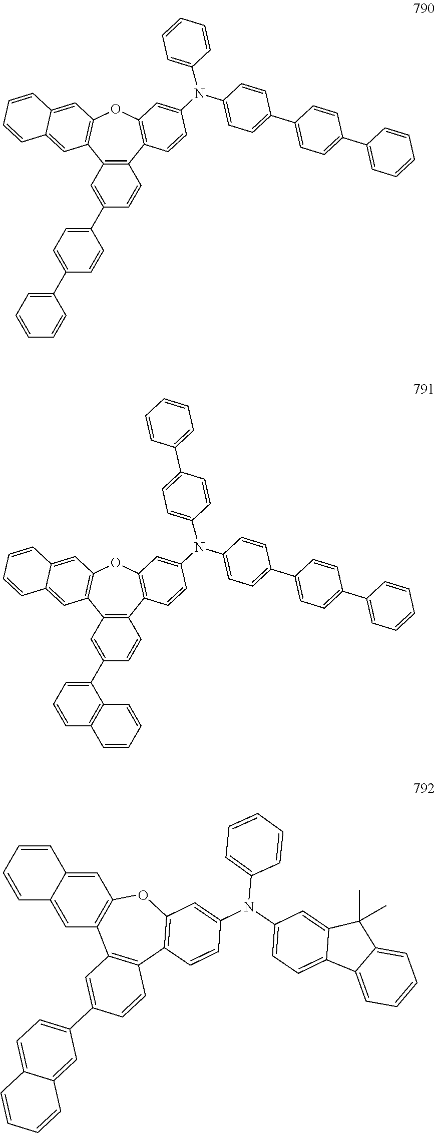 [SMILES: c1ccc(-c2ccc(-c3ccc(N(c4ccccc4)c4ccc5c(c4)Oc4cc6ccccc6cc4-c4cc(-c6ccc(-c7ccccc7)cc6)ccc4-5)cc3)cc2)cc1 ; c1ccc(-c2ccc(-c3ccc(N(c4ccc(-c5ccccc5)cc4)c4ccc5c(c4)Oc4cc6ccccc6cc4-c4cc(-c6cccc7ccccc67)ccc4-5)cc3)cc2)cc1 ; CC1(C)c2ccccc2-c2ccc(N(c3ccccc3)c3ccc4c(c3)Oc3cc5ccccc5cc3-c3cc(-c5ccc6ccccc6c5)ccc3-4)cc21]

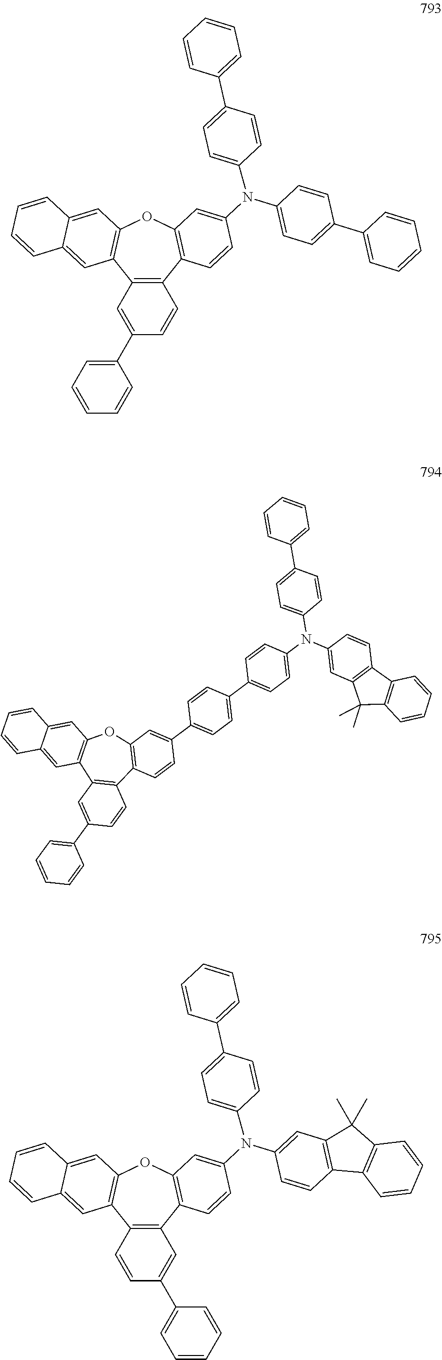 [SMILES: c1ccc(-c2ccc(N(c3ccc(-c4ccccc4)cc3)c3ccc4c(c3)Oc3cc5ccccc5cc3-c3cc(-c5ccccc5)ccc3-4)cc2)cc1 ; CC1(C)c2ccccc2-c2ccc(N(c3ccc(-c4ccccc4)cc3)c3ccc(-c4ccc(-c5ccc6c(c5)Oc5cc7ccccc7cc5-c5cc(-c7ccccc7)ccc5-6)cc4)cc3)cc21 ; CC1(C)c2ccccc2-c2ccc(N(c3ccc(-c4ccccc4)cc3)c3ccc4c(c3)Oc3cc5ccccc5cc3-c3ccc(-c5ccccc5)cc3-4)cc21]

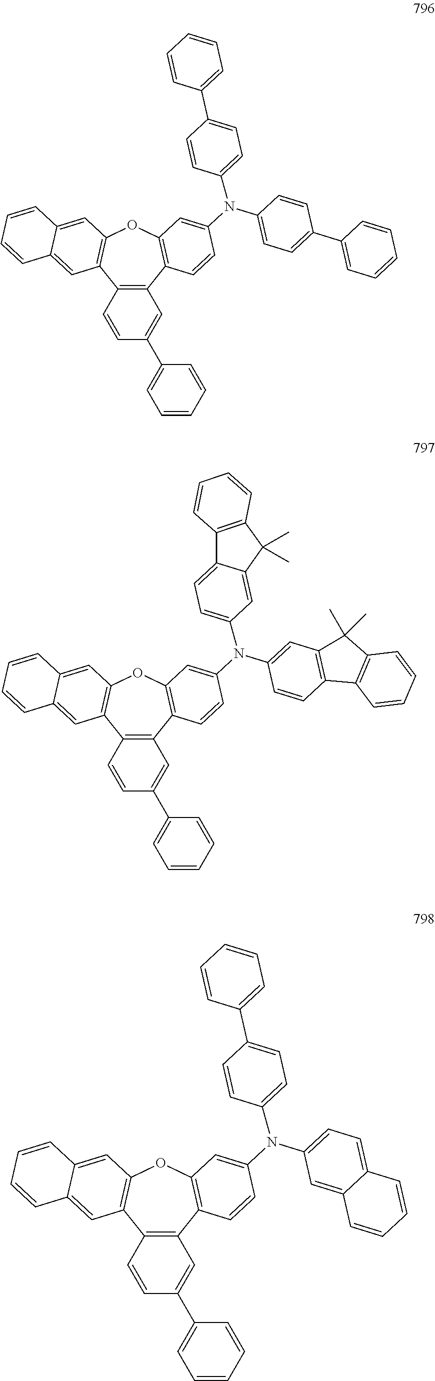 [SMILES: c1ccc(-c2ccc(N(c3ccc(-c4ccccc4)cc3)c3ccc4c(c3)Oc3cc5ccccc5cc3-c3ccc(-c5ccccc5)cc3-4)cc2)cc1 ; CC1(C)c2ccccc2-c2ccc(N(c3ccc4c(c3)Oc3cc5ccccc5cc3-c3ccc(-c5ccccc5)cc3-4)c3ccc4c(c3)C(C)(C)c3ccccc3-4)cc21 ; c1ccc(-c2ccc(N(c3ccc4c(c3)Oc3cc5ccccc5cc3-c3ccc(-c5ccccc5)cc3-4)c3ccc4ccccc4c3)cc2)cc1]

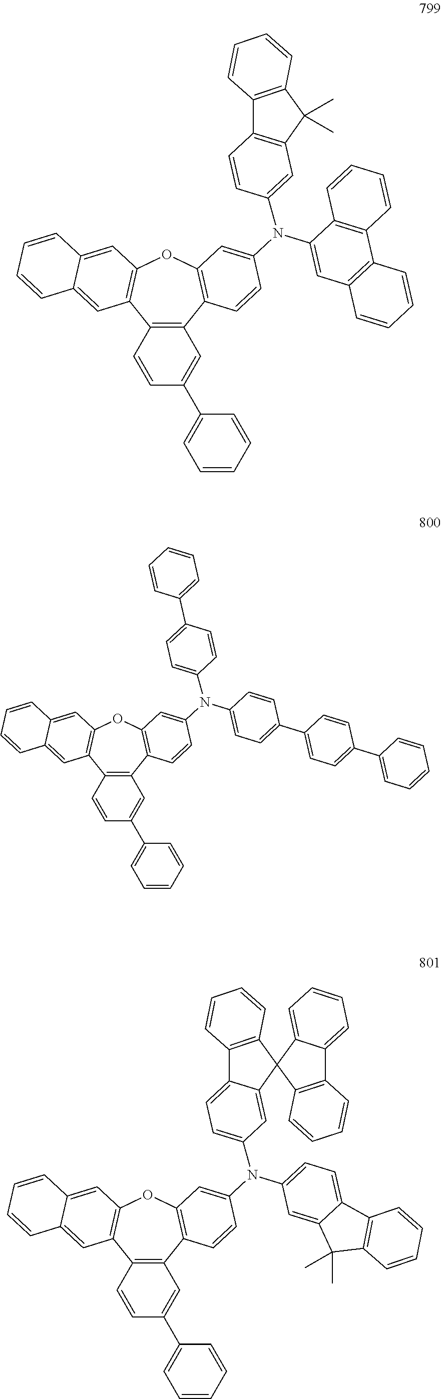 [SMILES: CC1(C)c2ccccc2-c2ccc(N(c3ccc4c(c3)Oc3cc5ccccc5cc3-c3ccc(-c5ccccc5)cc3-4)c3cc4ccccc4c4ccccc34)cc21 ; c1ccc(-c2ccc(-c3ccc(N(c4ccc(-c5ccccc5)cc4)c4ccc5c(c4)Oc4cc6ccccc6cc4-c4ccc(-c6ccccc6)cc4-5)cc3)cc2)cc1 ; CC1(C)c2ccccc2-c2ccc(N(c3ccc4c(c3)Oc3cc5ccccc5cc3-c3ccc(-c5ccccc5)cc3-4)c3ccc4c(c3)C3(c5ccccc5-c5ccccc53)c3ccccc3-4)cc21]

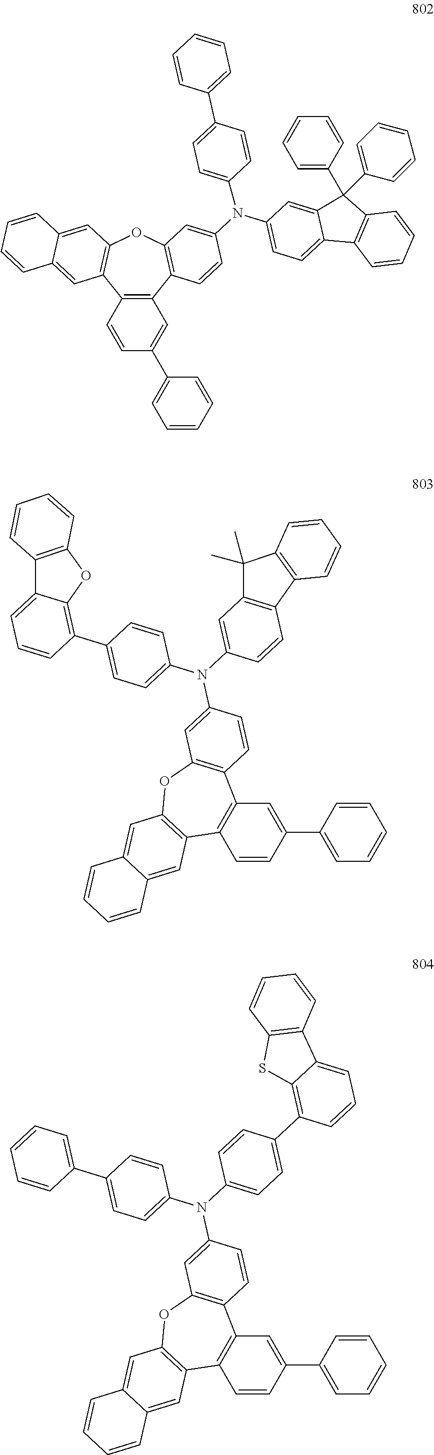 [SMILES: c1ccc(-c2ccc(N(c3ccc4c(c3)Oc3cc5ccccc5cc3-c3ccc(-c5ccccc5)cc3-4)c3ccc4c(c3)C(c3ccccc3)(c3ccccc3)c3ccccc3-4)cc2)cc1 ; CC1(C)c2ccccc2-c2ccc(N(c3ccc(-c4cccc5c4oc4ccccc45)cc3)c3ccc4c(c3)Oc3cc5ccccc5cc3-c3ccc(-c5ccccc5)cc3-4)cc21 ; c1ccc(-c2ccc(N(c3ccc(-c4cccc5c4sc4ccccc45)cc3)c3ccc4c(c3)Oc3cc5ccccc5cc3-c3ccc(-c5ccccc5)cc3-4)cc2)cc1]

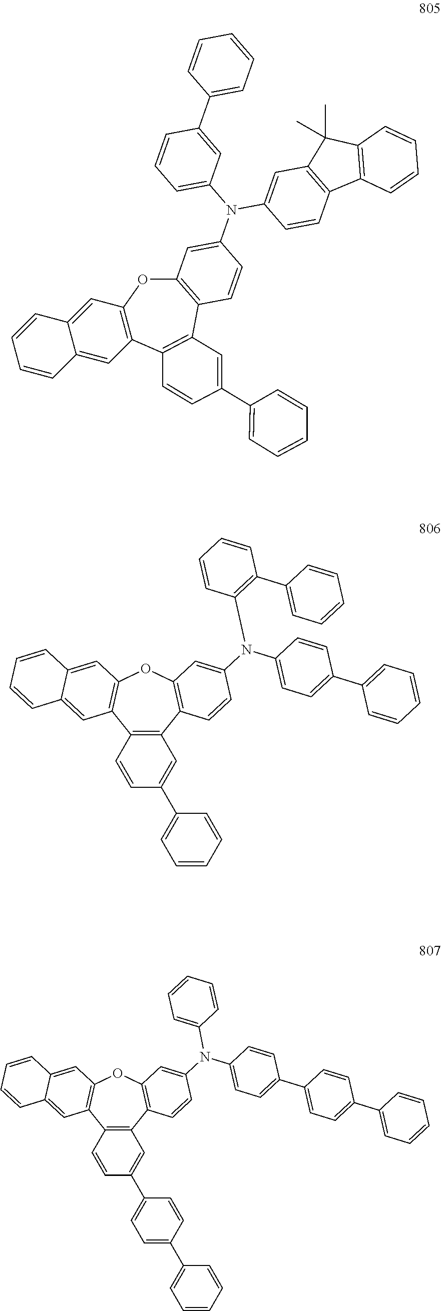 [SMILES: CC1(C)c2ccccc2-c2ccc(N(c3cccc(-c4ccccc4)c3)c3ccc4c(c3)Oc3cc5ccccc5cc3-c3ccc(-c5ccccc5)cc3-4)cc21 ; c1ccc(-c2ccc(N(c3ccc4c(c3)Oc3cc5ccccc5cc3-c3ccc(-c5ccccc5)cc3-4)c3ccccc3-c3ccccc3)cc2)cc1 ; c1ccc(-c2ccc(-c3ccc(N(c4ccccc4)c4ccc5c(c4)Oc4cc6ccccc6cc4-c4ccc(-c6ccc(-c7ccccc7)cc6)cc4-5)cc3)cc2)cc1]

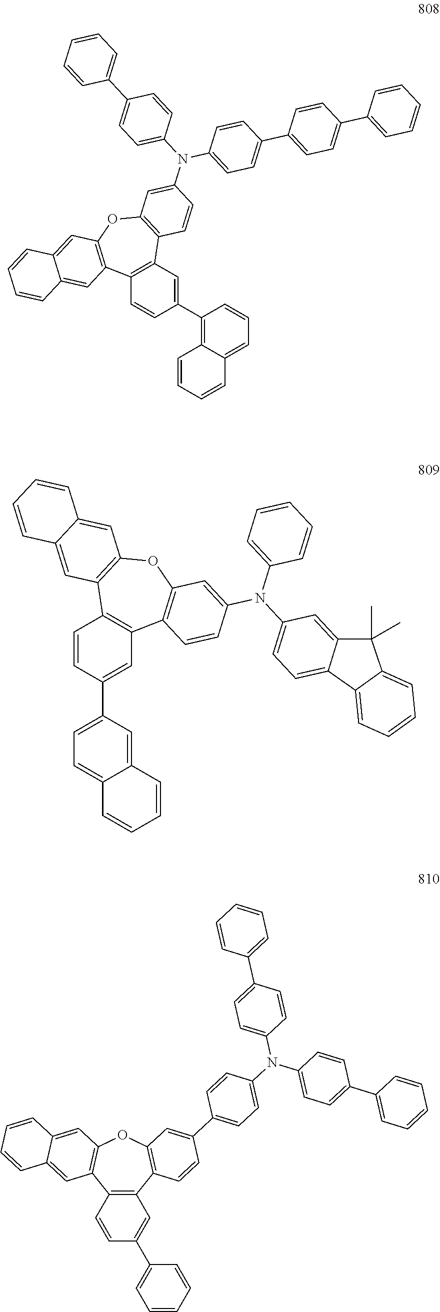 [SMILES: c1ccc(-c2ccc(-c3ccc(N(c4ccc(-c5ccccc5)cc4)c4ccc5c(c4)Oc4cc6ccccc6cc4-c4ccc(-c6cccc7ccccc67)cc4-5)cc3)cc2)cc1 ; CC1(C)c2ccccc2-c2ccc(N(c3ccccc3)c3ccc4c(c3)Oc3cc5ccccc5cc3-c3ccc(-c5ccc6ccccc6c5)cc3-4)cc21 ; c1ccc(-c2ccc(N(c3ccc(-c4ccccc4)cc3)c3ccc(-c4ccc5c(c4)Oc4cc6ccccc6cc4-c4ccc(-c6ccccc6)cc4-5)cc3)cc2)cc1]

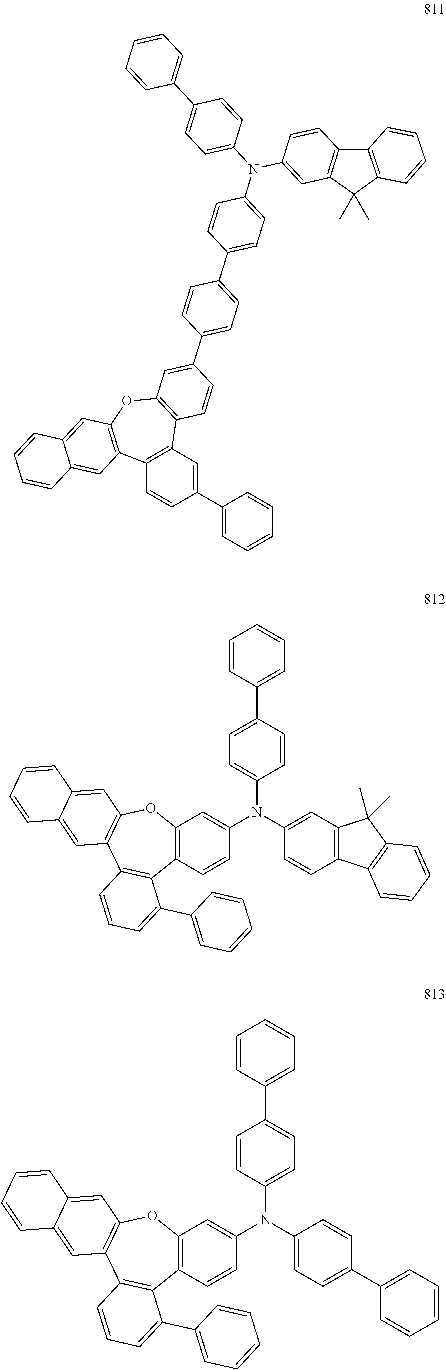 [SMILES: CC1(C)c2ccccc2-c2ccc(N(c3ccc(-c4ccccc4)cc3)c3ccc(-c4ccc(-c5ccc6c(c5)Oc5cc7ccccc7cc5-c5ccc(-c7ccccc7)cc5-6)cc4)cc3)cc21 ; CC1(C)c2ccccc2-c2ccc(N(c3ccc(-c4ccccc4)cc3)c3ccc4c(c3)Oc3cc5ccccc5cc3-c3cccc(-c5ccccc5)c3-4)cc21 ; c1ccc(-c2ccc(N(c3ccc(-c4ccccc4)cc3)c3ccc4c(c3)Oc3cc5ccccc5cc3-c3cccc(-c5ccccc5)c3-4)cc2)cc1]

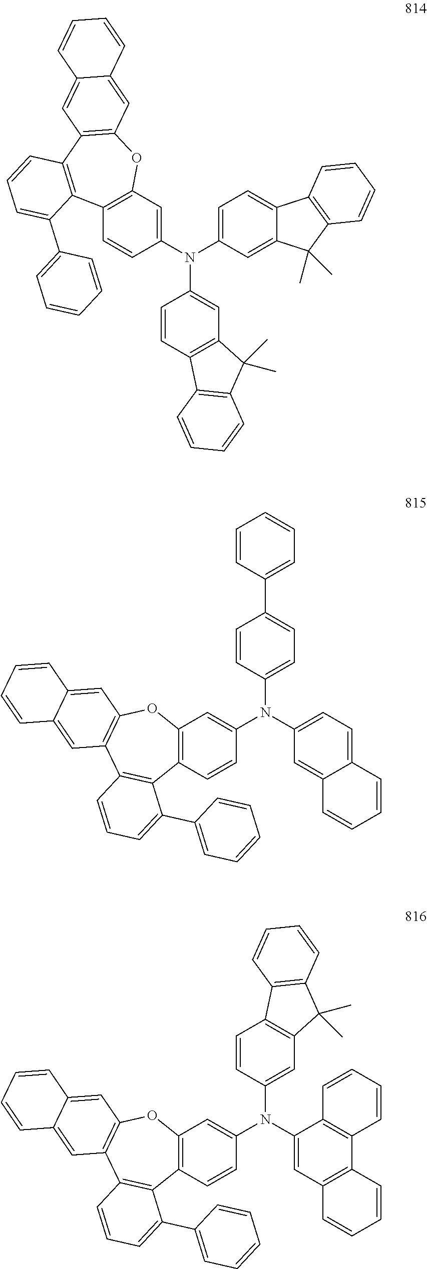 [SMILES: CC1(C)c2ccccc2-c2ccc(N(c3ccc4c(c3)Oc3cc5ccccc5cc3-c3cccc(-c5ccccc5)c3-4)c3ccc4c(c3)C(C)(C)c3ccccc3-4)cc21 ; c1ccc(-c2ccc(N(c3ccc4c(c3)Oc3cc5ccccc5cc3-c3cccc(-c5ccccc5)c3-4)c3ccc4ccccc4c3)cc2)cc1 ; CC1(C)c2ccccc2-c2ccc(N(c3ccc4c(c3)Oc3cc5ccccc5cc3-c3cccc(-c5ccccc5)c3-4)c3cc4ccccc4c4ccccc34)cc21]

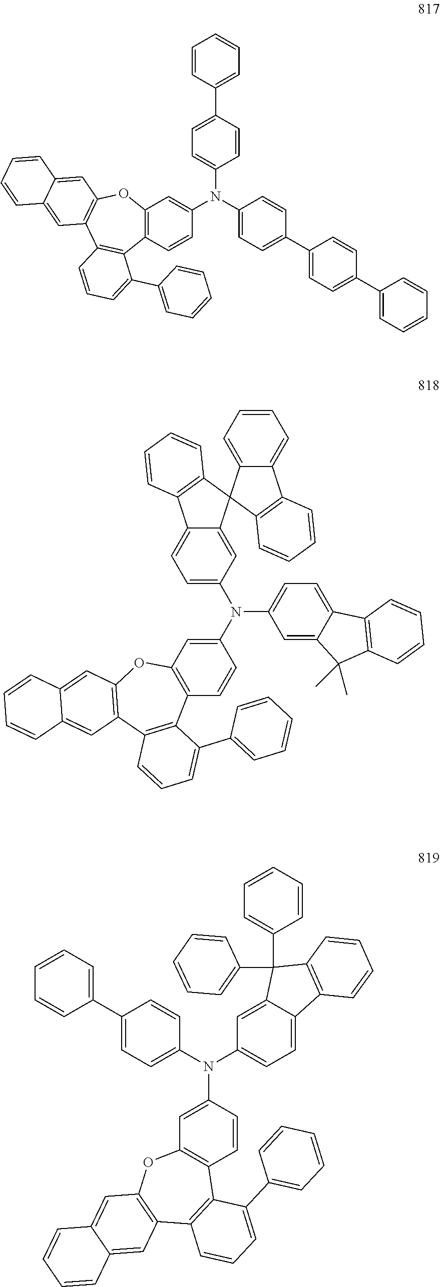 [SMILES: c1ccc(-c2ccc(-c3ccc(N(c4ccc(-c5ccccc5)cc4)c4ccc5c(c4)Oc4cc6ccccc6cc4-c4cccc(-c6ccccc6)c4-5)cc3)cc2)cc1 ; CC1(C)c2ccccc2-c2ccc(N(c3ccc4c(c3)Oc3cc5ccccc5cc3-c3cccc(-c5ccccc5)c3-4)c3ccc4c(c3)C3(c5ccccc5-c5ccccc53)c3ccccc3-4)cc21 ; c1ccc(-c2ccc(N(c3ccc4c(c3)Oc3cc5ccccc5cc3-c3cccc(-c5ccccc5)c3-4)c3ccc4c(c3)C(c3ccccc3)(c3ccccc3)c3ccccc3-4)cc2)cc1]

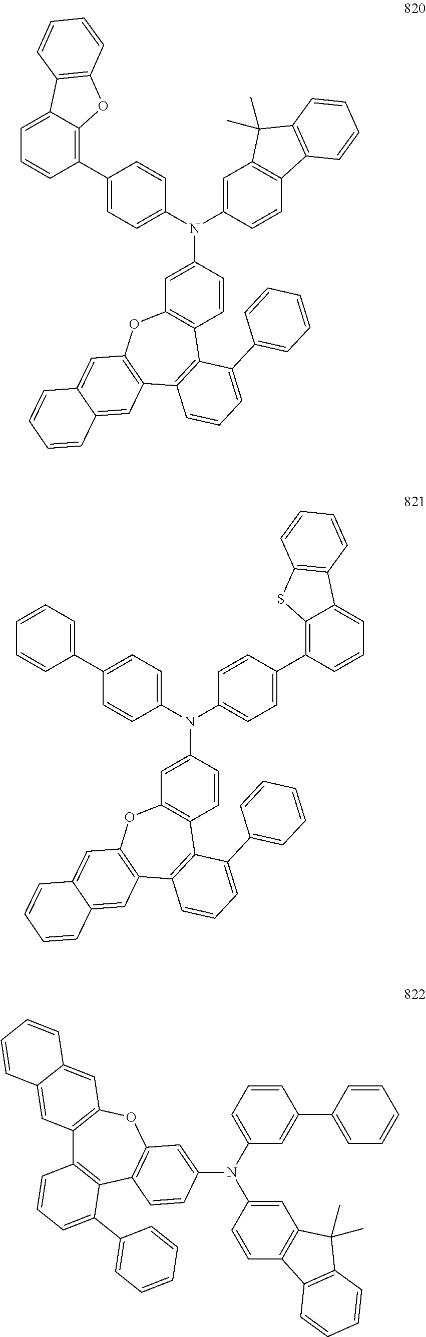 [SMILES: CC1(C)c2ccccc2-c2ccc(N(c3ccc(-c4cccc5c4oc4ccccc45)cc3)c3ccc4c(c3)Oc3cc5ccccc5cc3-c3cccc(-c5ccccc5)c3-4)cc21 ; c1ccc(-c2ccc(N(c3ccc(-c4cccc5c4sc4ccccc45)cc3)c3ccc4c(c3)Oc3cc5ccccc5cc3-c3cccc(-c5ccccc5)c3-4)cc2)cc1 ; CC1(C)c2ccccc2-c2ccc(N(c3cccc(-c4ccccc4)c3)c3ccc4c(c3)Oc3cc5ccccc5cc3-c3cccc(-c5ccccc5)c3-4)cc21]

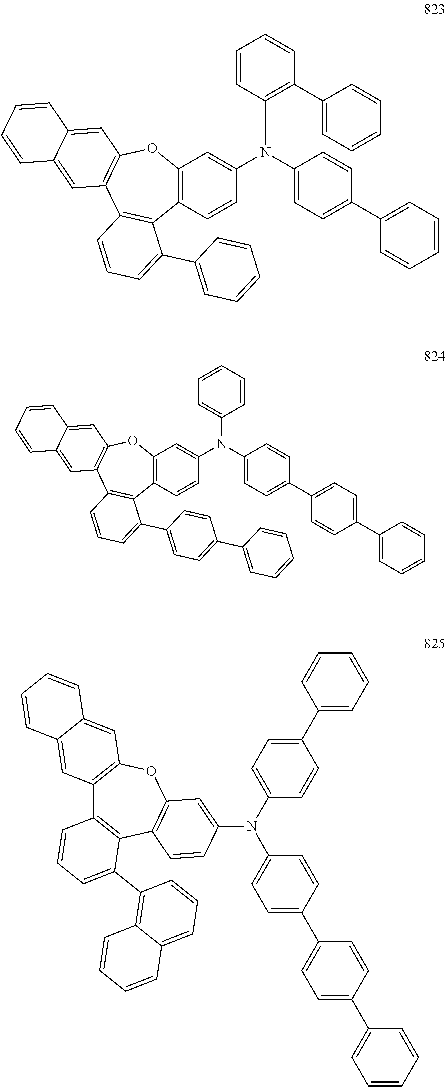 [SMILES: c1ccc(-c2ccc(N(c3ccc4c(c3)Oc3cc5ccccc5cc3-c3cccc(-c5ccccc5)c3-4)c3ccccc3-c3ccccc3)cc2)cc1 ; c1ccc(-c2ccc(-c3ccc(N(c4ccccc4)c4ccc5c(c4)Oc4cc6ccccc6cc4-c4cccc(-c6ccc(-c7ccccc7)cc6)c4-5)cc3)cc2)cc1 ; c1ccc(-c2ccc(-c3ccc(N(c4ccc(-c5ccccc5)cc4)c4ccc5c(c4)Oc4cc6ccccc6cc4-c4cccc(-c6cccc7ccccc67)c4-5)cc3)cc2)cc1]

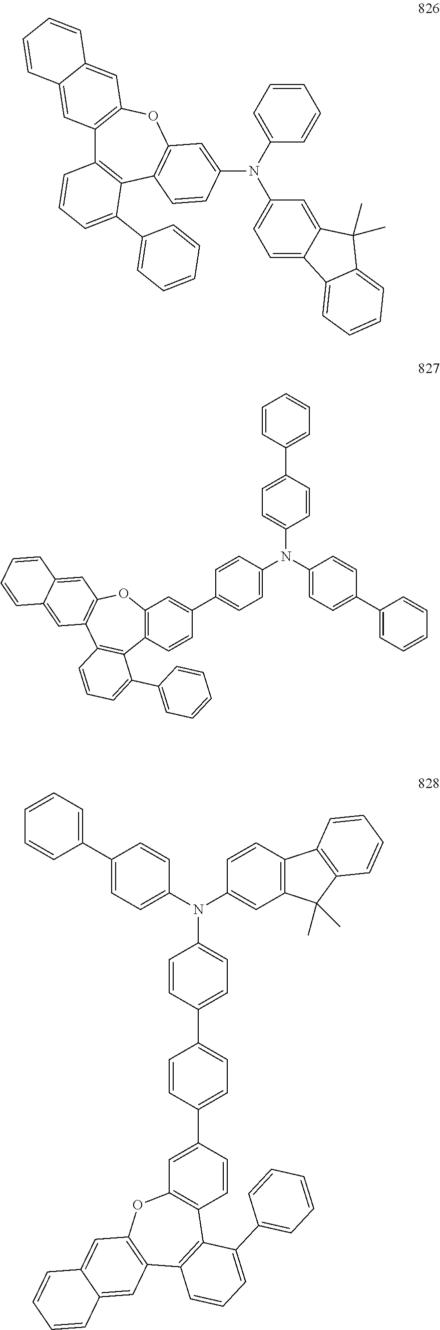 [SMILES: CC1(C)c2ccccc2-c2ccc(N(c3ccccc3)c3ccc4c(c3)Oc3cc5ccccc5cc3-c3cccc(-c5ccccc5)c3-4)cc21 ; c1ccc(-c2ccc(N(c3ccc(-c4ccccc4)cc3)c3ccc(-c4ccc5c(c4)Oc4cc6ccccc6cc4-c4cccc(-c6ccccc6)c4-5)cc3)cc2)cc1 ; CC1(C)c2ccccc2-c2ccc(N(c3ccc(-c4ccccc4)cc3)c3ccc(-c4ccc(-c5ccc6c(c5)Oc5cc7ccccc7cc5-c5cccc(-c7ccccc7)c5-6)cc4)cc3)cc21]

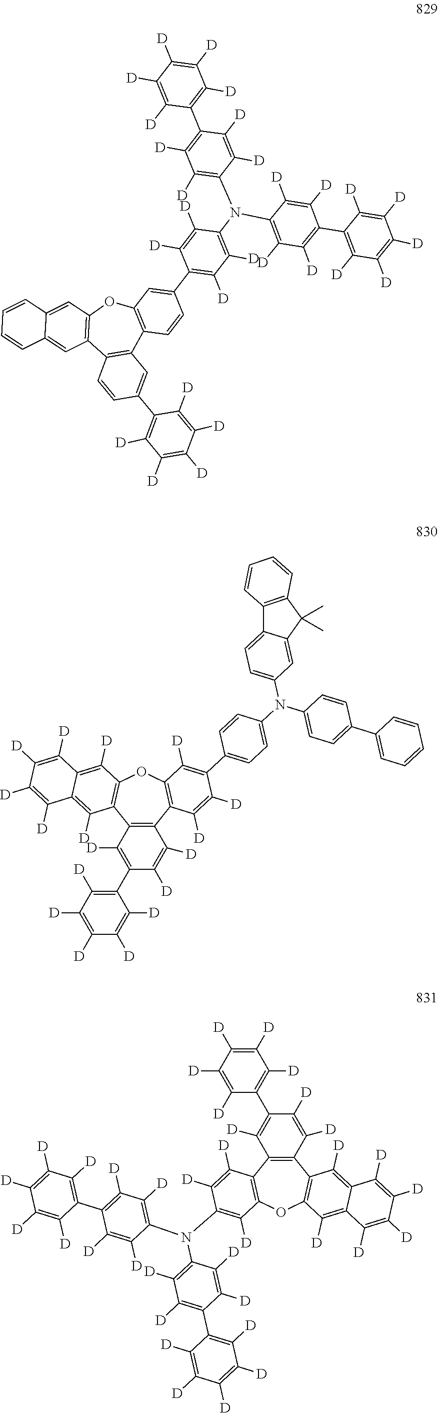

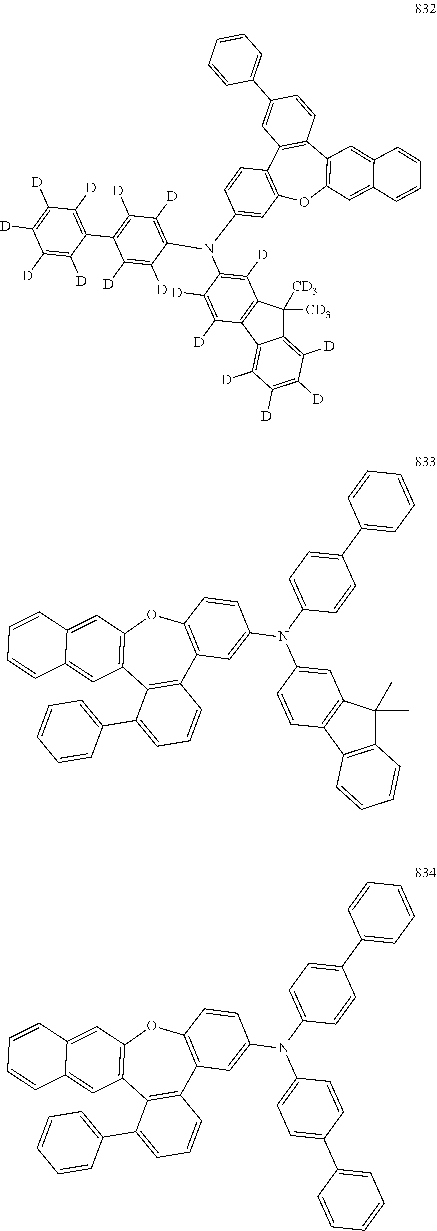 [SMILES: [2H]c1c([2H])c([2H])c(-c2c([2H])c([2H])c(N(c3ccc4c(c3)Oc3cc5ccccc5cc3-c3ccc(-c5ccccc5)cc3-4)c3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H] ; CC1(C)c2ccccc2-c2ccc(N(c3ccc(-c4ccccc4)cc3)c3ccc4c(c3)-c3cccc(-c5ccccc5)c3-c3cc5ccccc5cc3O4)cc21 ; c1ccc(-c2ccc(N(c3ccc(-c4ccccc4)cc3)c3ccc4c(c3)-c3cccc(-c5ccccc5)c3-c3cc5ccccc5cc3O4)cc2)cc1]

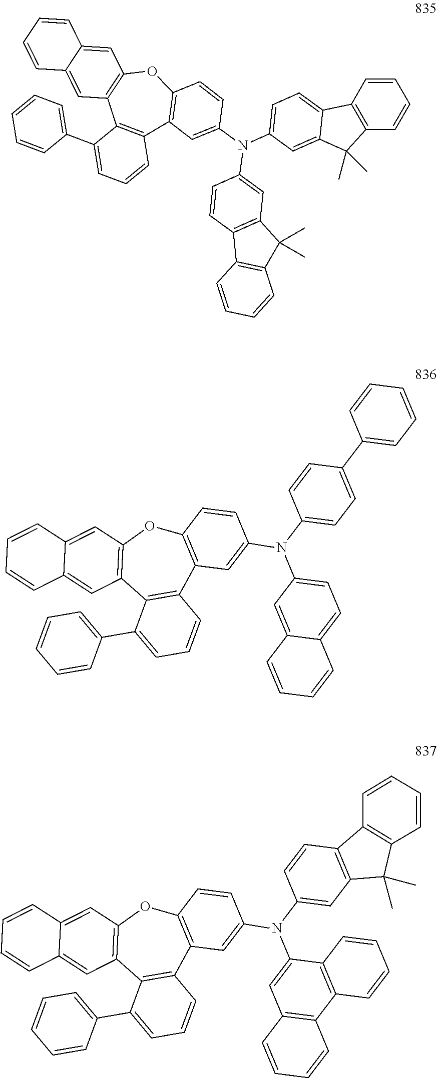 [SMILES: CC1(C)c2ccccc2-c2ccc(N(c3ccc4c(c3)-c3cccc(-c5ccccc5)c3-c3cc5ccccc5cc3O4)c3ccc4c(c3)C(C)(C)c3ccccc3-4)cc21 ; c1ccc(-c2ccc(N(c3ccc4c(c3)-c3cccc(-c5ccccc5)c3-c3cc5ccccc5cc3O4)c3ccc4ccccc4c3)cc2)cc1 ; CC1(C)c2ccccc2-c2ccc(N(c3ccc4c(c3)-c3cccc(-c5ccccc5)c3-c3cc5ccccc5cc3O4)c3cc4ccccc4c4ccccc34)cc21]

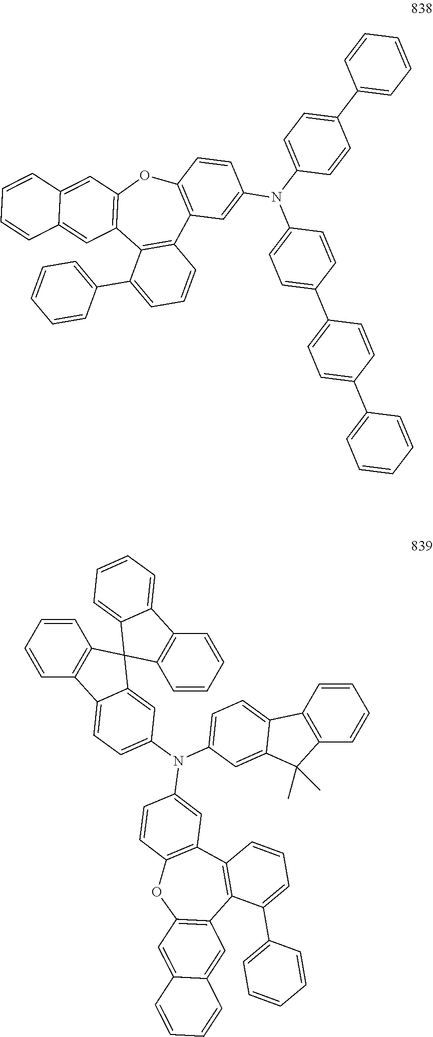 [SMILES: c1ccc(-c2ccc(-c3ccc(N(c4ccc(-c5ccccc5)cc4)c4ccc5c(c4)-c4cccc(-c6ccccc6)c4-c4cc6ccccc6cc4O5)cc3)cc2)cc1 ; CC1(C)c2ccccc2-c2ccc(N(c3ccc4c(c3)-c3cccc(-c5ccccc5)c3-c3cc5ccccc5cc3O4)c3ccc4c(c3)C3(c5ccccc5-c5ccccc53)c3ccccc3-4)cc21]

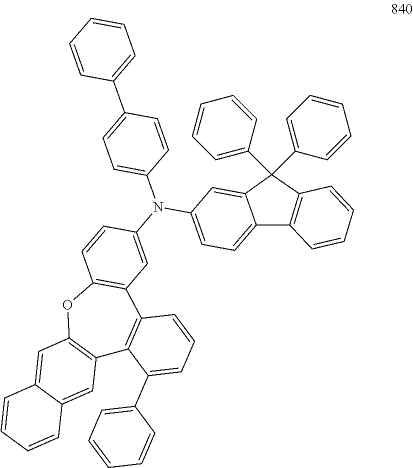 [SMILES: c1ccc(-c2ccc(N(c3ccc4c(c3)-c3cccc(-c5ccccc5)c3-c3cc5ccccc5cc3O4)c3ccc4c(c3)C(c3ccccc3)(c3ccccc3)c3ccccc3-4)cc2)cc1]

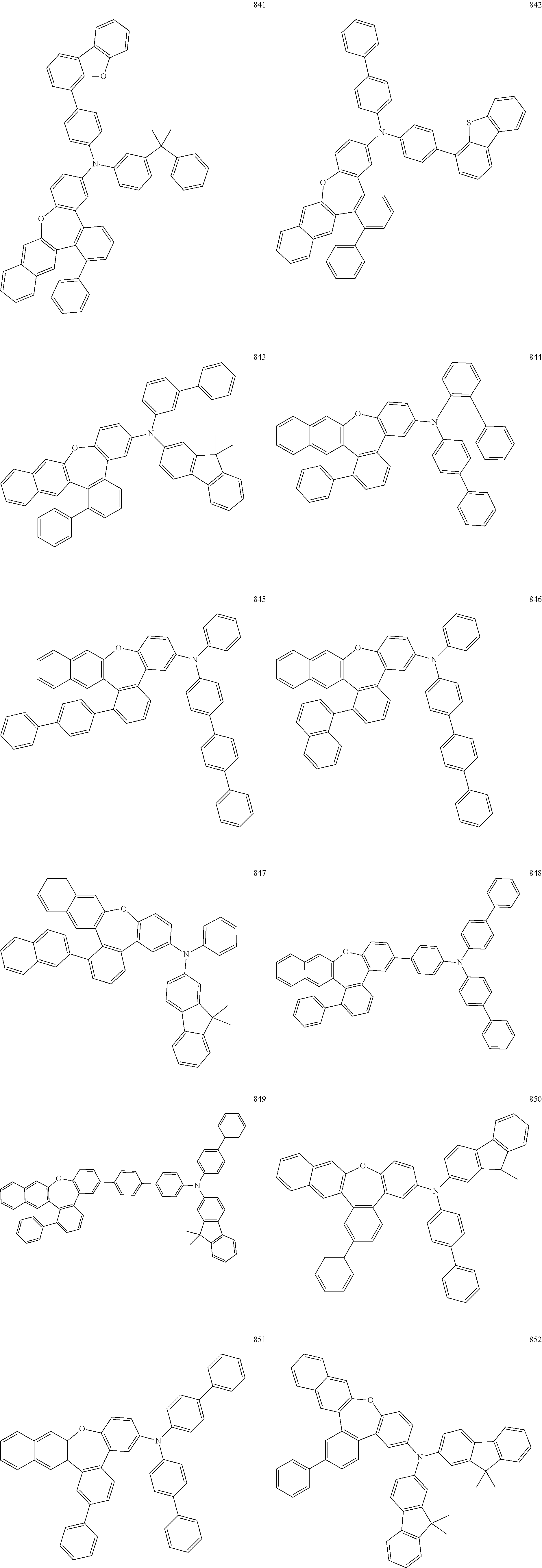

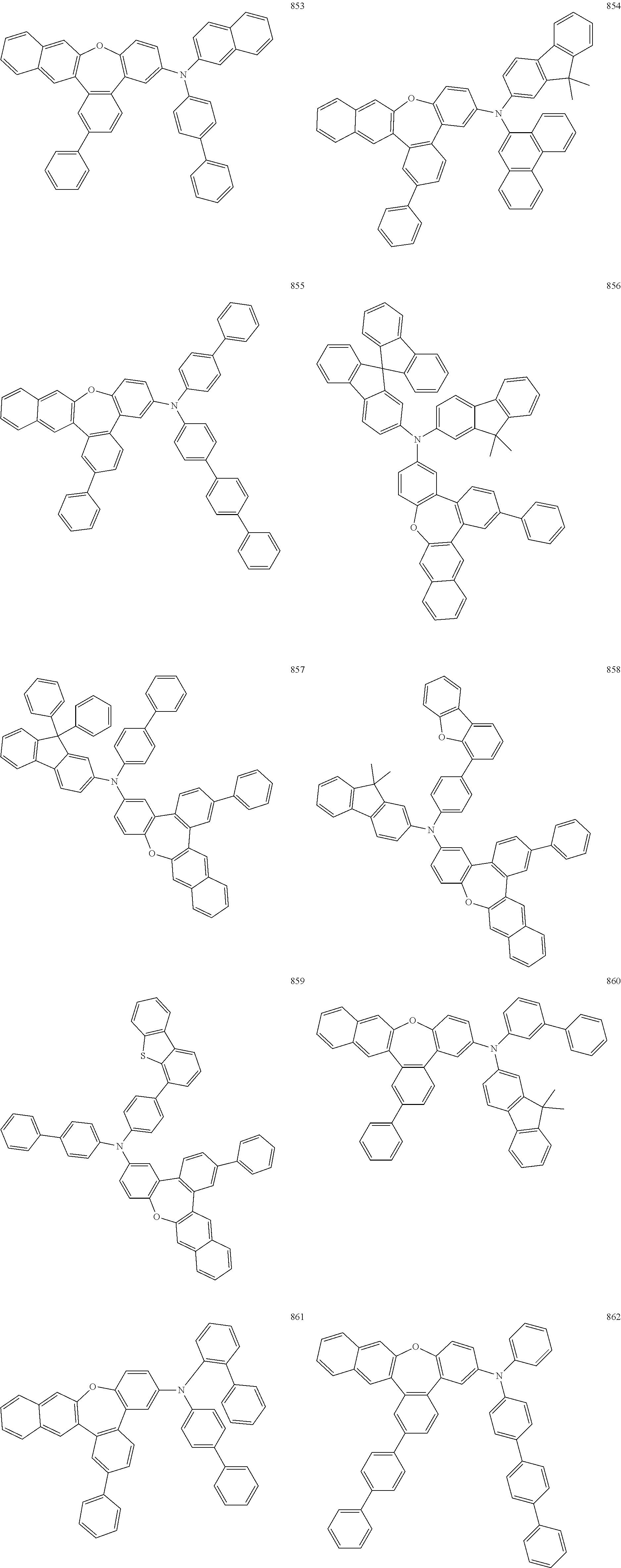

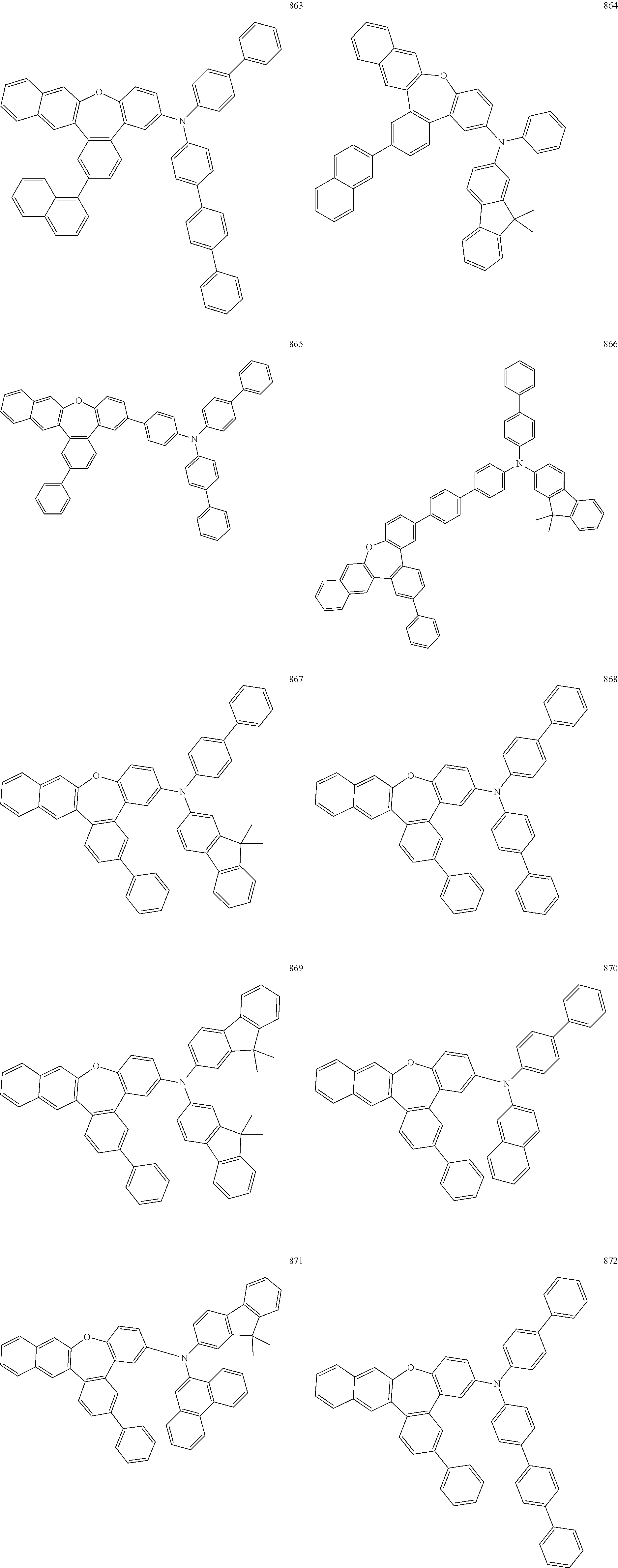

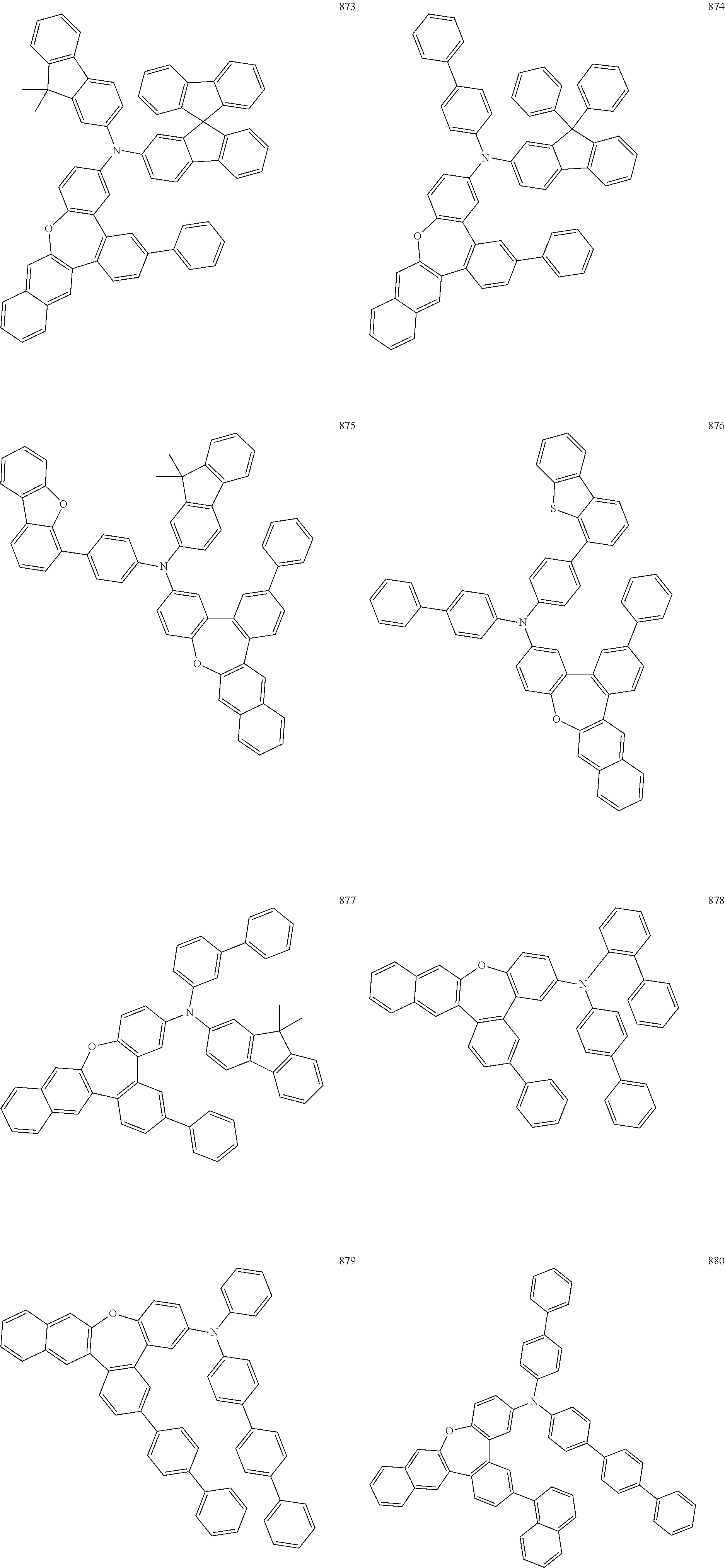

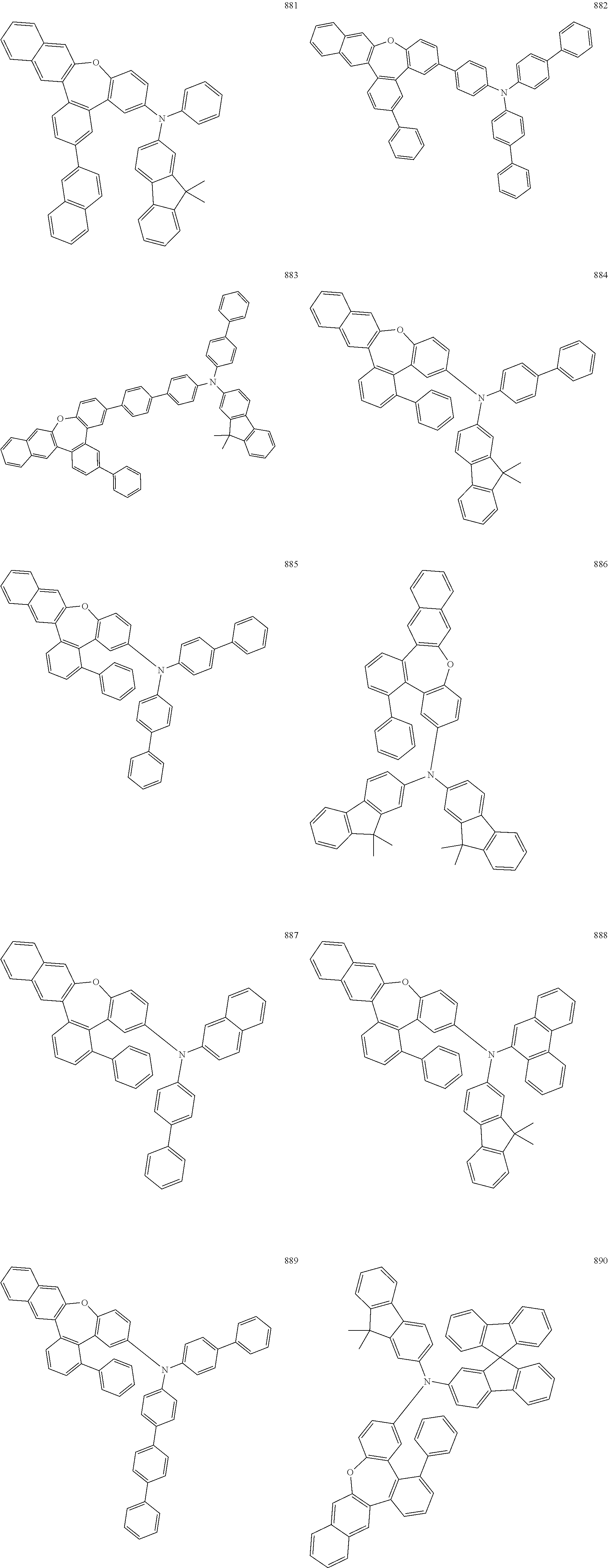

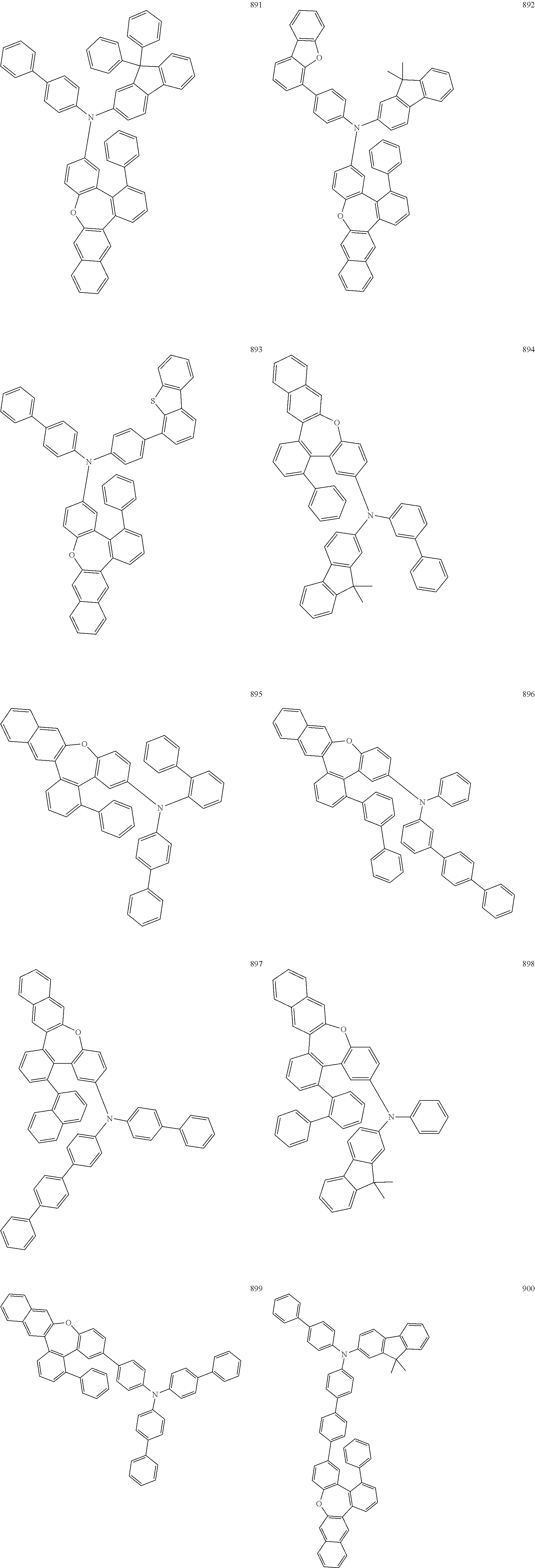

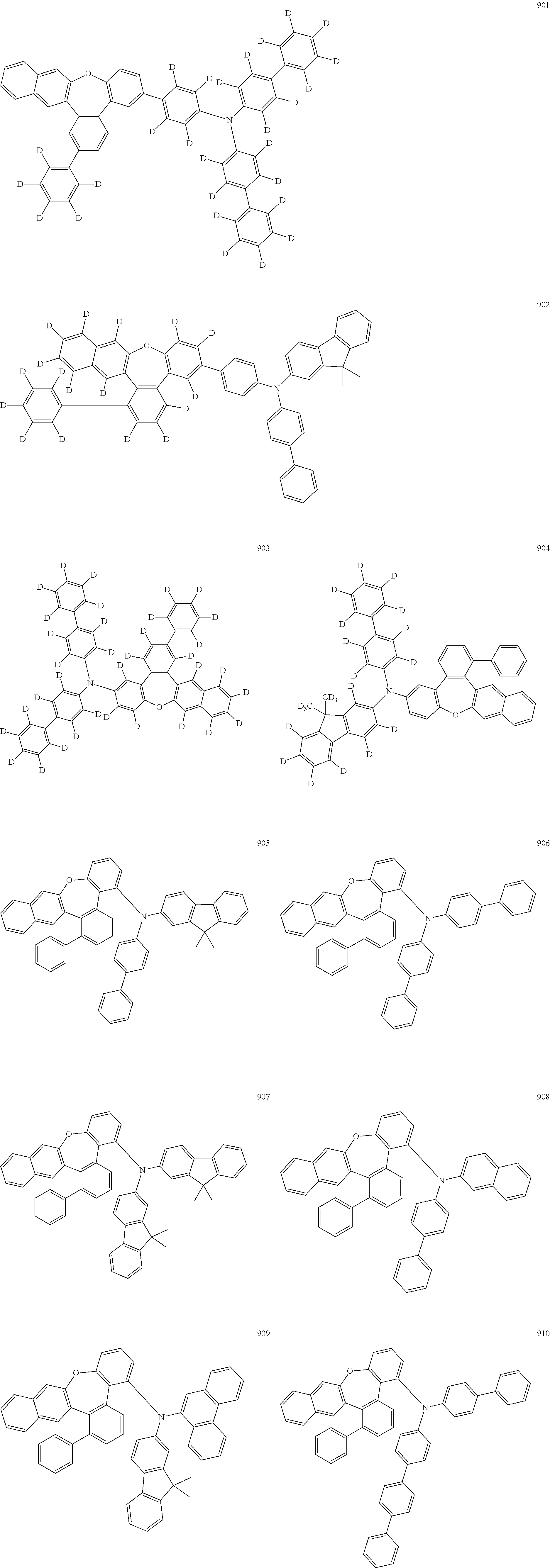

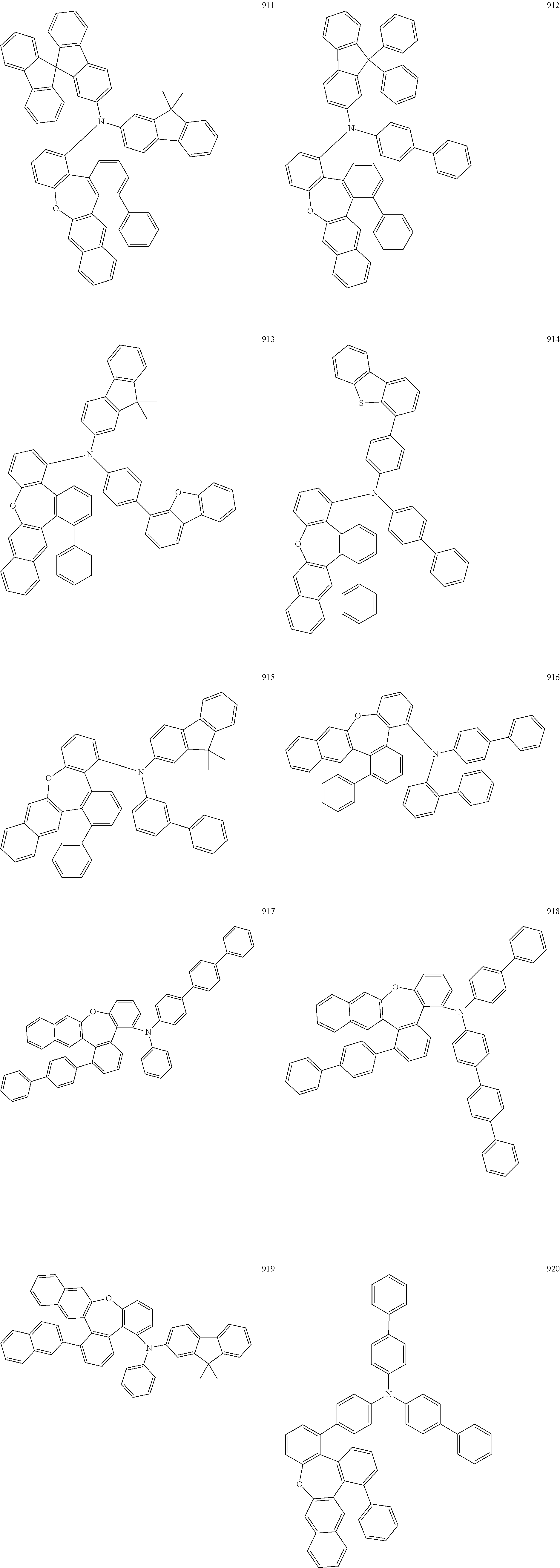

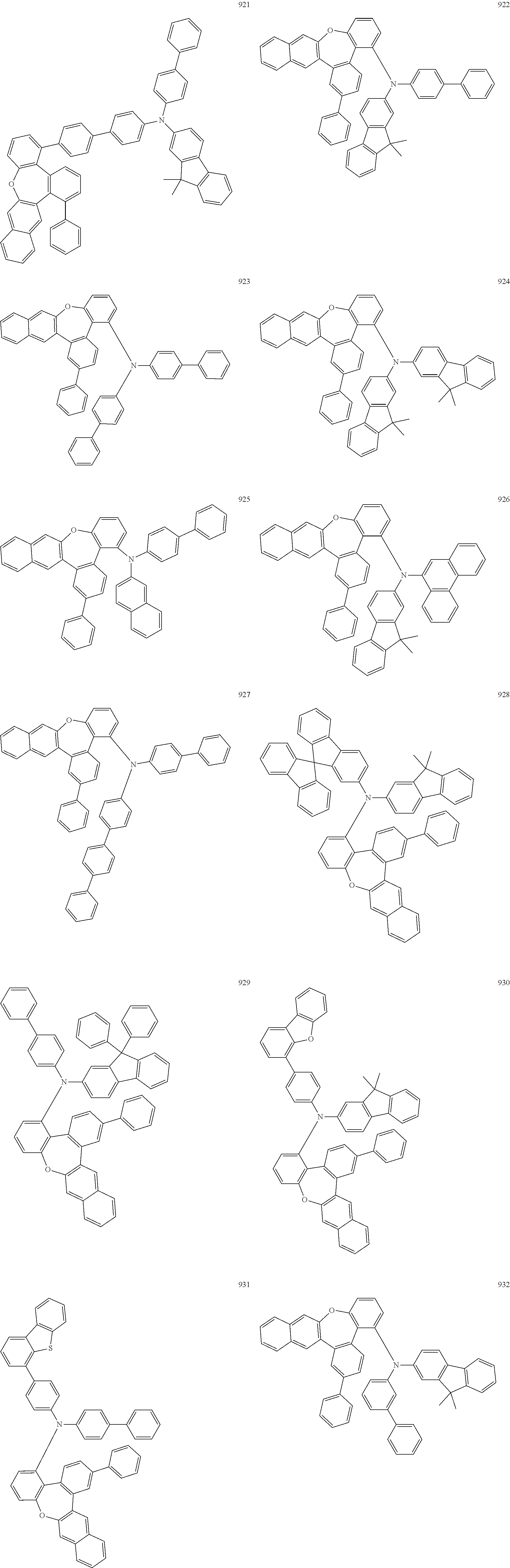

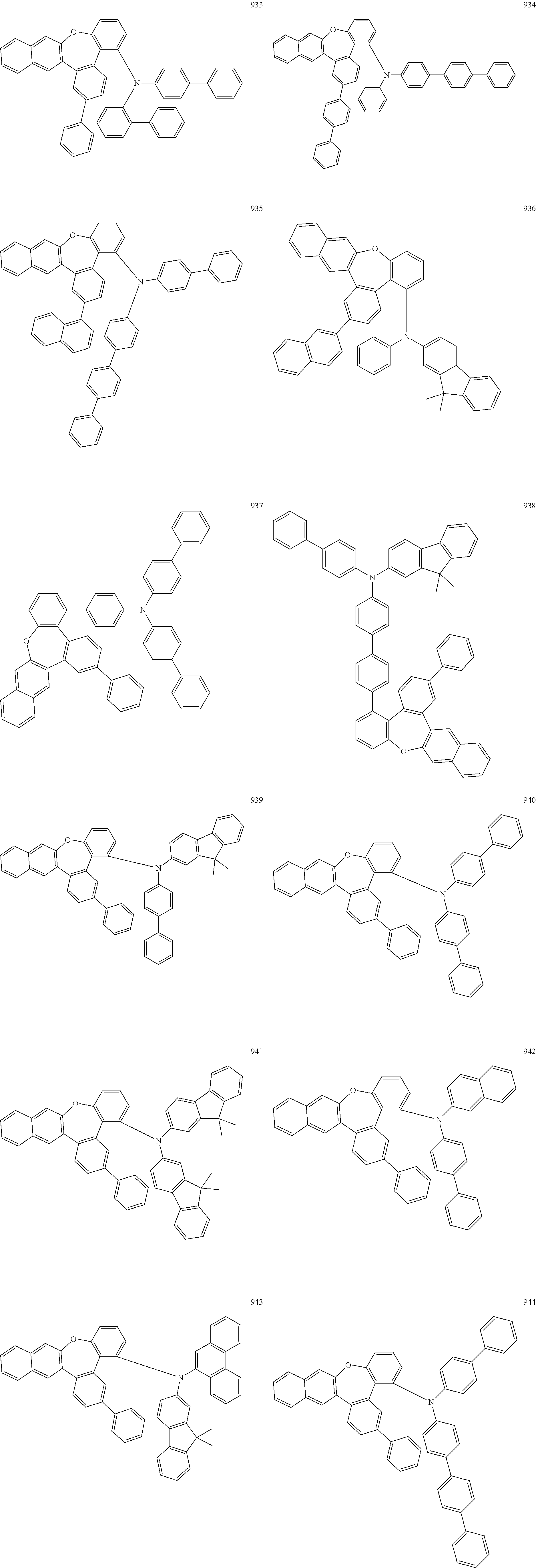

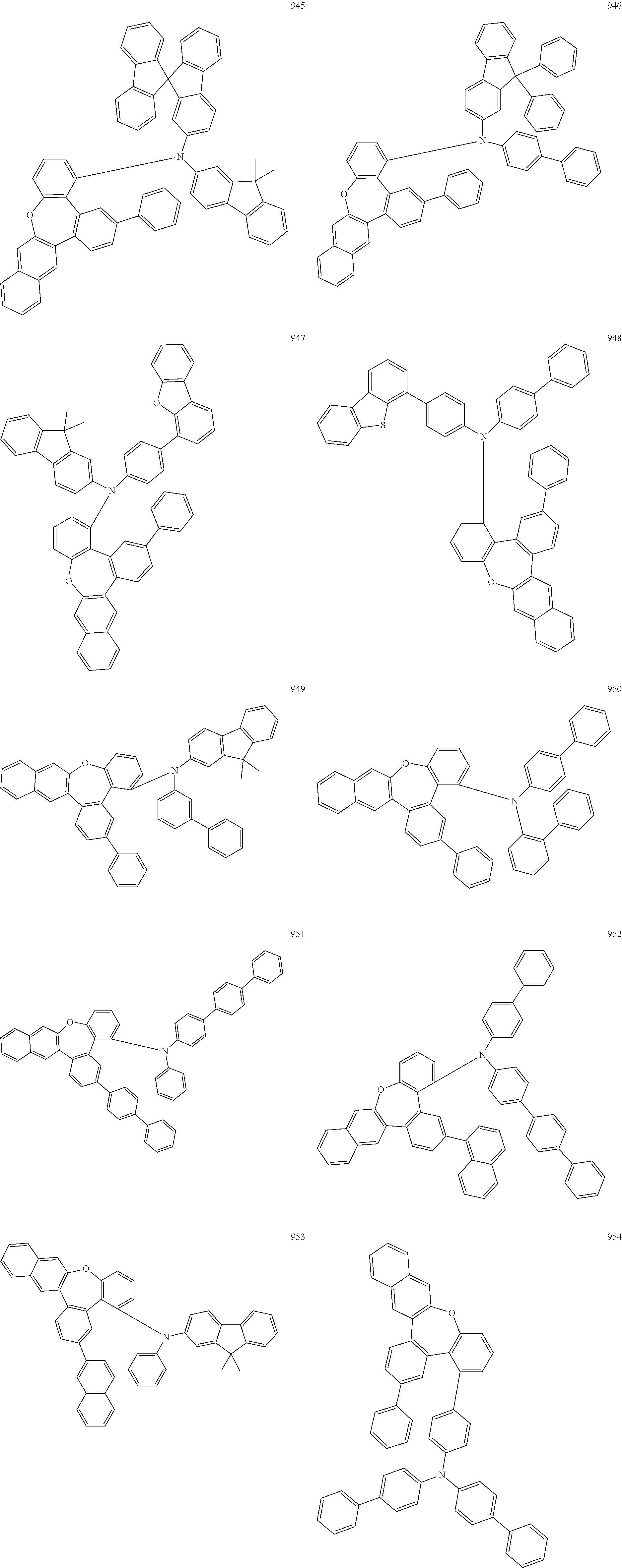

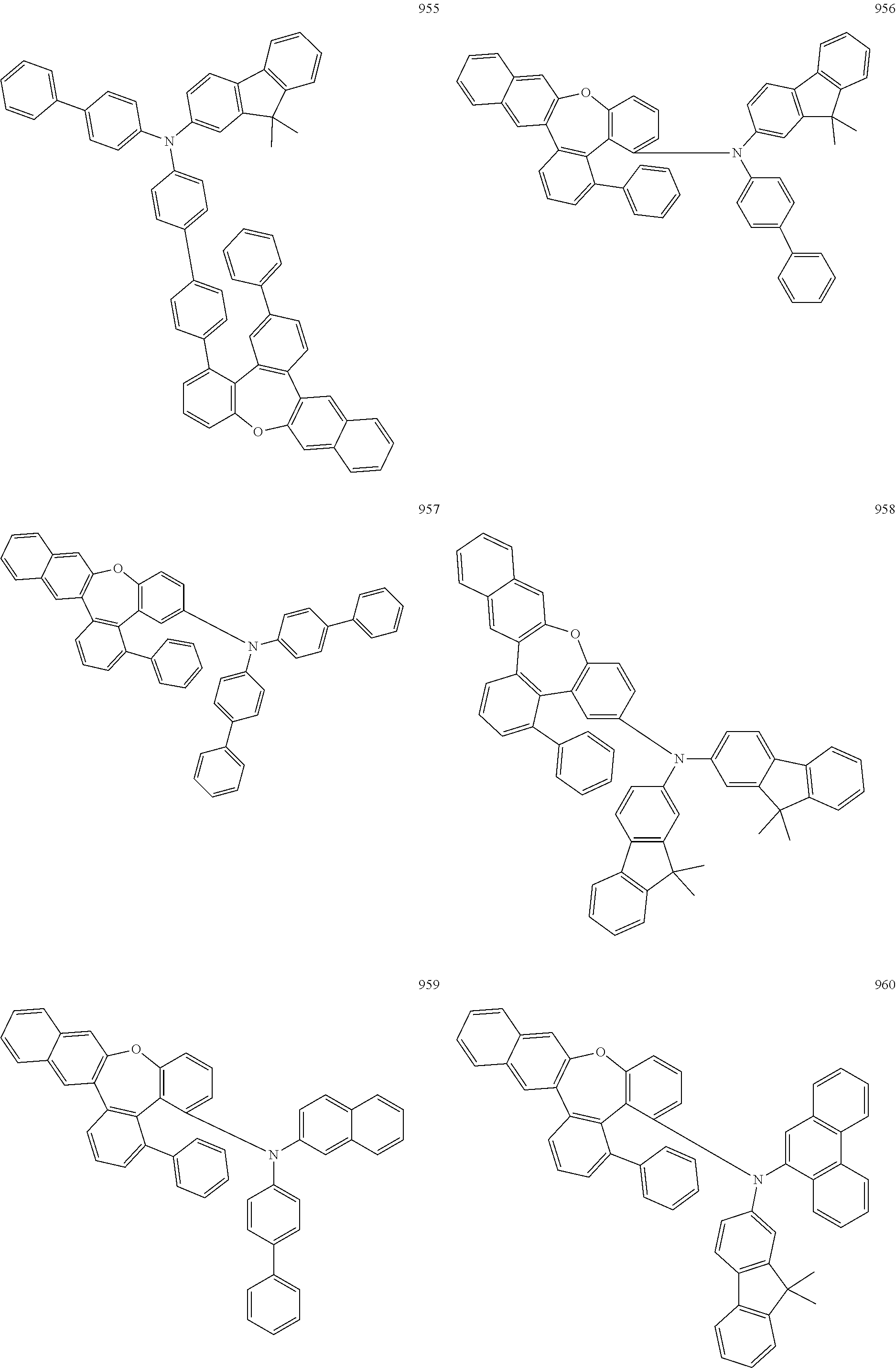

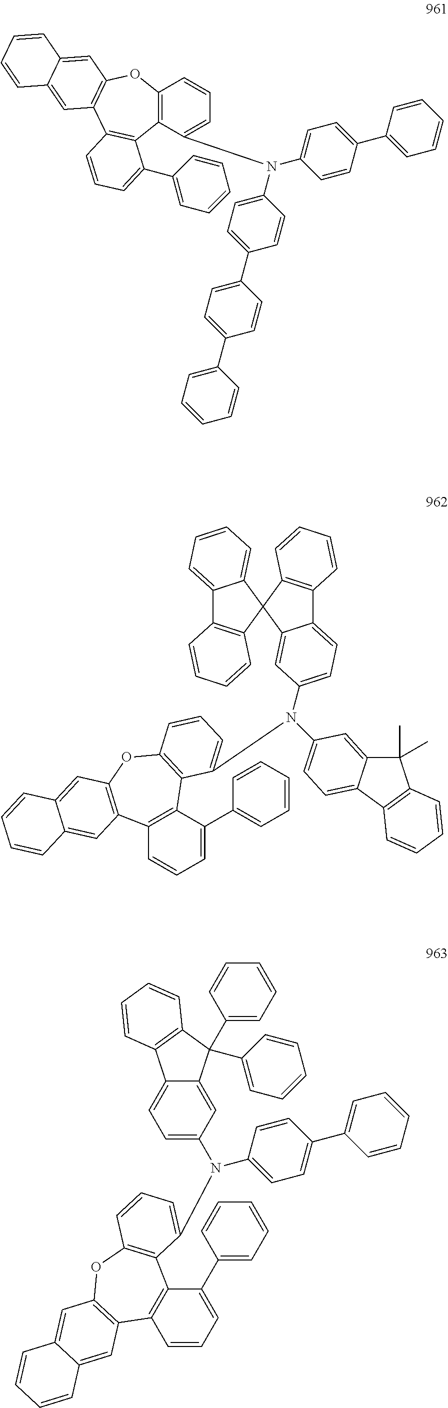 [SMILES: c1ccc(-c2ccc(-c3ccc(N(c4ccc(-c5ccccc5)cc4)c4cccc5c4-c4c(-c6ccccc6)cccc4-c4cc6ccccc6cc4O5)cc3)cc2)cc1 ; CC1(C)c2ccccc2-c2ccc(N(c3ccc4c(c3)C3(c5ccccc5-c5ccccc53)c3ccccc3-4)c3cccc4c3-c3c(-c5ccccc5)cccc3-c3cc5ccccc5cc3O4)cc21 ; c1ccc(-c2ccc(N(c3ccc4c(c3)C(c3ccccc3)(c3ccccc3)c3ccccc3-4)c3cccc4c3-c3c(-c5ccccc5)cccc3-c3cc5ccccc5cc3O4)cc2)cc1]

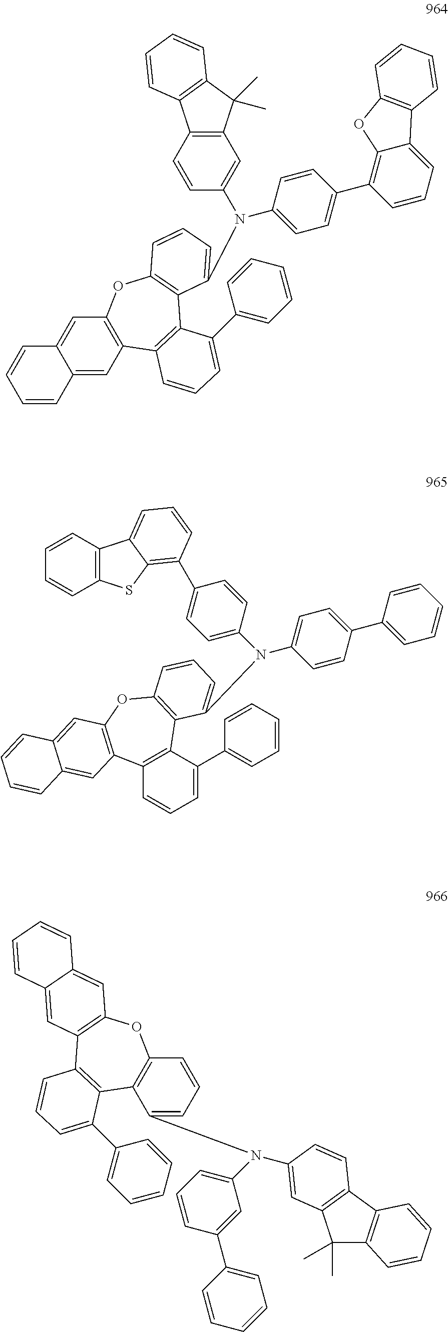 [SMILES: CC1(C)c2ccccc2-c2ccc(N(c3ccc(-c4cccc5c4oc4ccccc45)cc3)c3cccc4c3-c3c(-c5ccccc5)cccc3-c3cc5ccccc5cc3O4)cc21 ; c1ccc(-c2ccc(N(c3ccc(-c4cccc5c4sc4ccccc45)cc3)c3cccc4c3-c3c(-c5ccccc5)cccc3-c3cc5ccccc5cc3O4)cc2)cc1 ; CC1(C)c2ccccc2-c2ccc(N(c3cccc(-c4ccccc4)c3)c3cccc4c3-c3c(-c5ccccc5)cccc3-c3cc5ccccc5cc3O4)cc21]

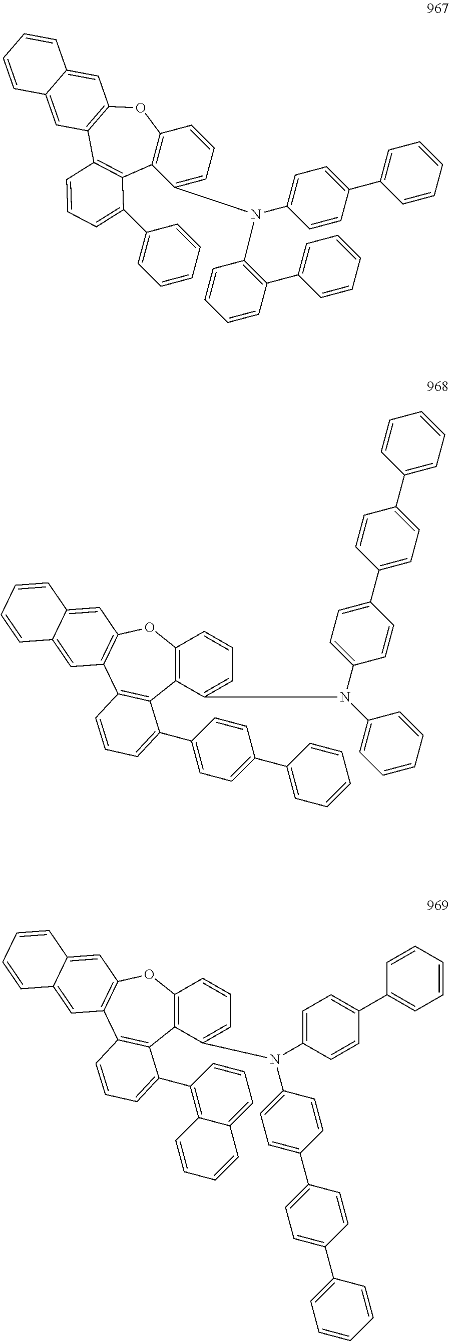 [SMILES: c1ccc(-c2ccc(N(c3ccccc3-c3ccccc3)c3cccc4c3-c3c(-c5ccccc5)cccc3-c3cc5ccccc5cc3O4)cc2)cc1 ; c1ccc(-c2ccc(-c3ccc(N(c4ccccc4)c4cccc5c4-c4c(-c6ccc(-c7ccccc7)cc6)cccc4-c4cc6ccccc6cc4O5)cc3)cc2)cc1 ; c1ccc(-c2ccc(-c3ccc(N(c4ccc(-c5ccccc5)cc4)c4cccc5c4-c4c(cccc4-c4cccc6ccccc46)-c4cc6ccccc6cc4O5)cc3)cc2)cc1]

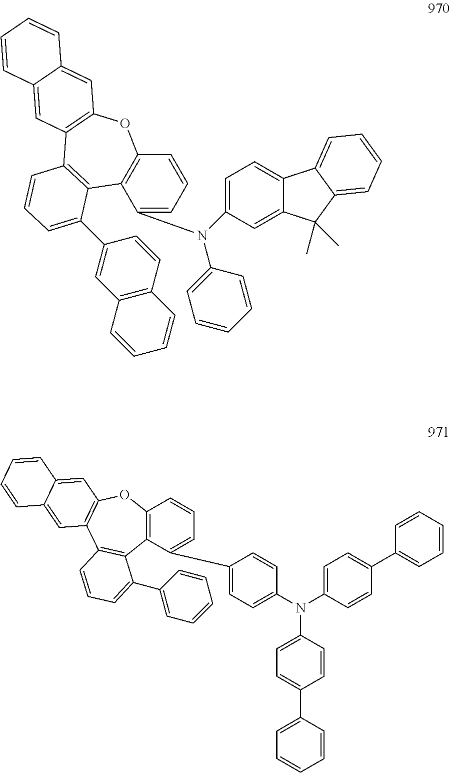 [SMILES: CC1(C)c2ccccc2-c2ccc(N(c3ccccc3)c3cccc4c3-c3c(-c5ccc6ccccc6c5)cccc3-c3cc5ccccc5cc3O4)cc21 ; c1ccc(-c2ccc(N(c3ccc(-c4ccccc4)cc3)c3ccc(-c4cccc5c4-c4c(-c6ccccc6)cccc4-c4cc6ccccc6cc4O5)cc3)cc2)cc1]

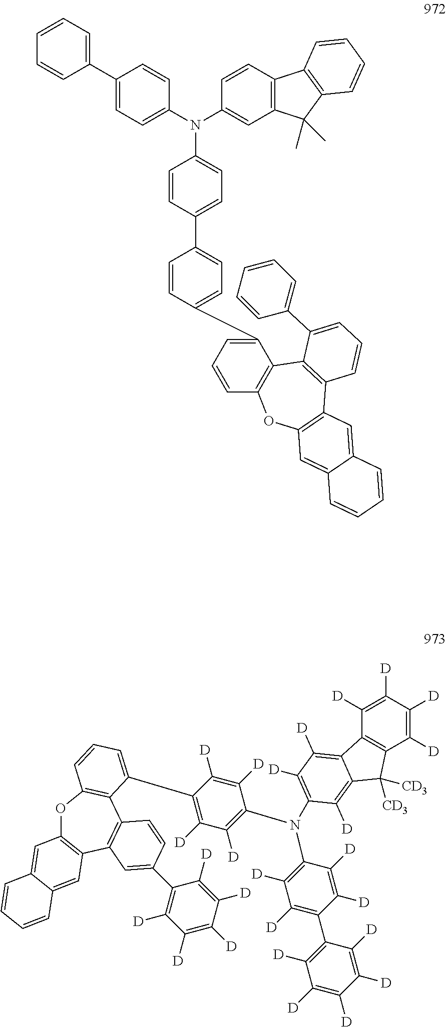 [SMILES: CC1(C)c2ccccc2-c2ccc(N(c3ccc(-c4ccccc4)cc3)c3ccc(-c4ccc(-c5cccc6c5-c5c(-c7ccccc7)cccc5-c5cc7ccccc7cc5O6)cc4)cc3)cc21 ; [2H]c1c([2H])c([2H])c(-c2ccc3c(c2)-c2cc4ccccc4cc2Oc2cccc(-c4c([2H])c([2H])c(N(c5c([2H])c([2H])c(-c6c([2H])c([2H])c([2H])c([2H])c6[2H])c([2H])c5[2H])c5c([2H])c([2H])c6c(c5[2H])C(C([2H])([2H])[2H])(C([2H])([2H])[2H])c5c([2H])c([2H])c([2H])c([2H])c5-6)c([2H])c4[2H])c2-3)c([2H])c1[2H]]

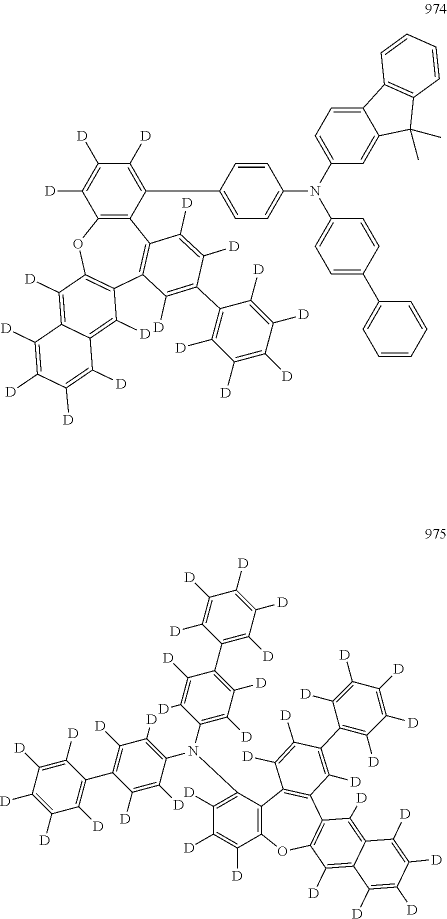 [SMILES: [2H]c1c([2H])c([2H])c(-c2c([2H])c([2H])c3c(c2[2H])-c2c(c([2H])c4c([2H])c([2H])c([2H])c([2H])c4c2[2H])Oc2c([2H])c([2H])c([2H])c(-c4ccc(N(c5ccc(-c6ccccc6)cc5)c5ccc6c(c5)C(C)(C)c5ccccc5-6)cc4)c2-3)c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c([2H])c4c3-c3c([2H])c([2H])c(-c5c([2H])c([2H])c([2H])c([2H])c5[2H])c([2H])c3-c3c(c([2H])c5c([2H])c([2H])c([2H])c([2H])c5c3[2H])O4)c([2H])c2[2H])c([2H])c1[2H]]

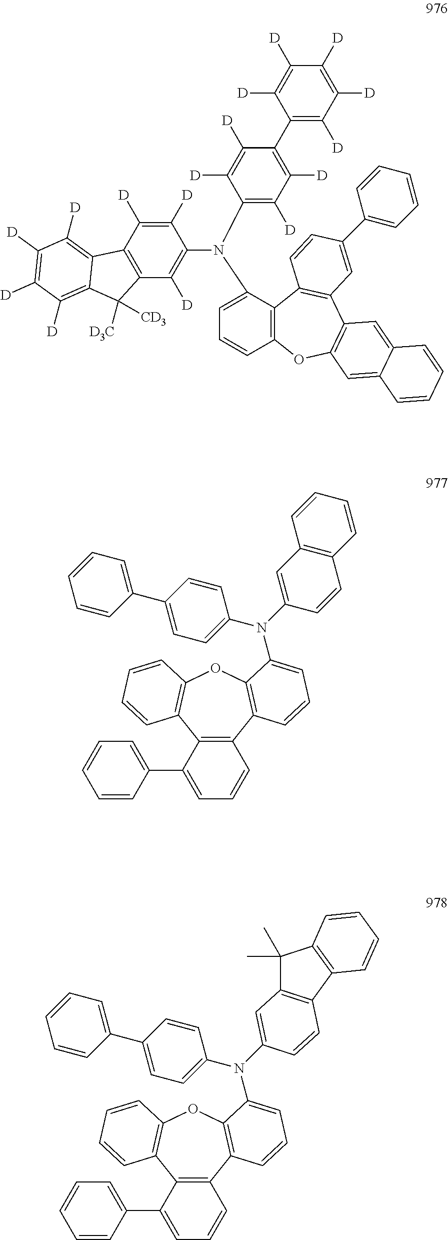 [SMILES: [2H]c1c([2H])c([2H])c(-c2c([2H])c([2H])c(N(c3cccc4c3-c3ccc(-c5ccccc5)cc3-c3cc5ccccc5cc3O4)c3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H] ; c1ccc(-c2ccc(N(c3ccc4ccccc4c3)c3cccc4c3Oc3ccccc3-c3c(-c5ccccc5)cccc3-4)cc2)cc1 ; CC1(C)c2ccccc2-c2ccc(N(c3ccc(-c4ccccc4)cc3)c3cccc4c3Oc3ccccc3-c3c(-c5ccccc5)cccc3-4)cc21]

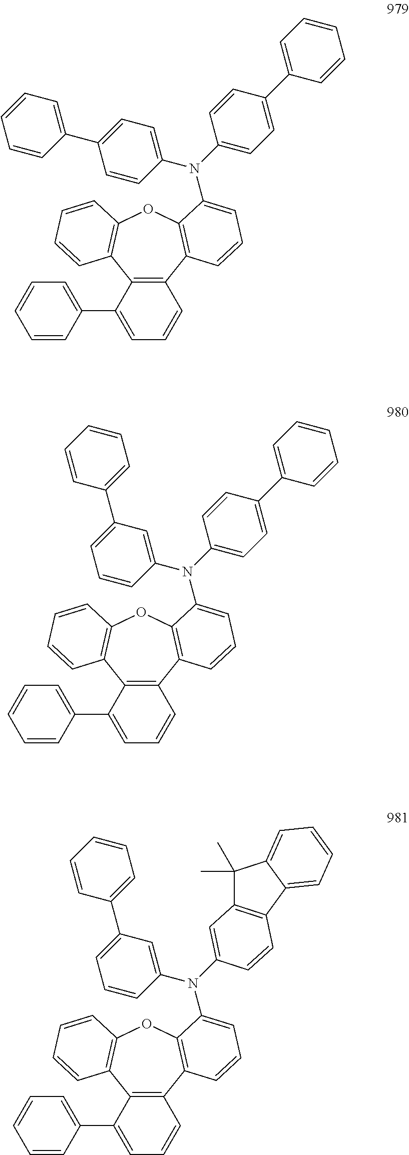 [SMILES: c1ccc(-c2ccc(N(c3ccc(-c4ccccc4)cc3)c3cccc4c3Oc3ccccc3-c3c(-c5ccccc5)cccc3-4)cc2)cc1 ; c1ccc(-c2ccc(N(c3cccc(-c4ccccc4)c3)c3cccc4c3Oc3ccccc3-c3c(-c5ccccc5)cccc3-4)cc2)cc1 ; CC1(C)c2ccccc2-c2ccc(N(c3cccc(-c4ccccc4)c3)c3cccc4c3Oc3ccccc3-c3c(-c5ccccc5)cccc3-4)cc21]

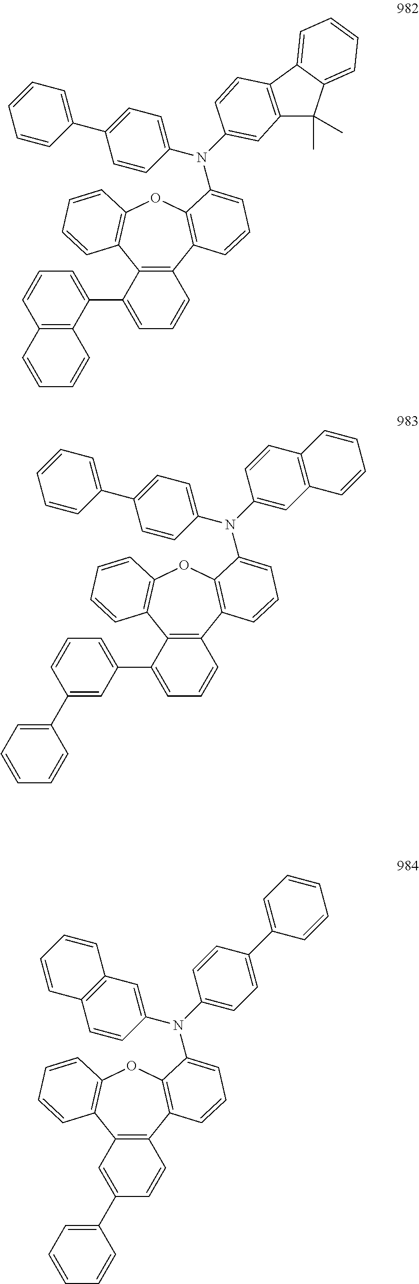 [SMILES: CC1(C)c2ccccc2-c2ccc(N(c3ccc(-c4ccccc4)cc3)c3cccc4c3Oc3ccccc3-c3c-4cccc3-c3cccc4ccccc34)cc21 ; c1ccc(-c2ccc(N(c3ccc4ccccc4c3)c3cccc4c3Oc3ccccc3-c3c(-c5cccc(-c6ccccc6)c5)cccc3-4)cc2)cc1 ; c1ccc(-c2ccc(N(c3ccc4ccccc4c3)c3cccc4c3Oc3ccccc3-c3cc(-c5ccccc5)ccc3-4)cc2)cc1]

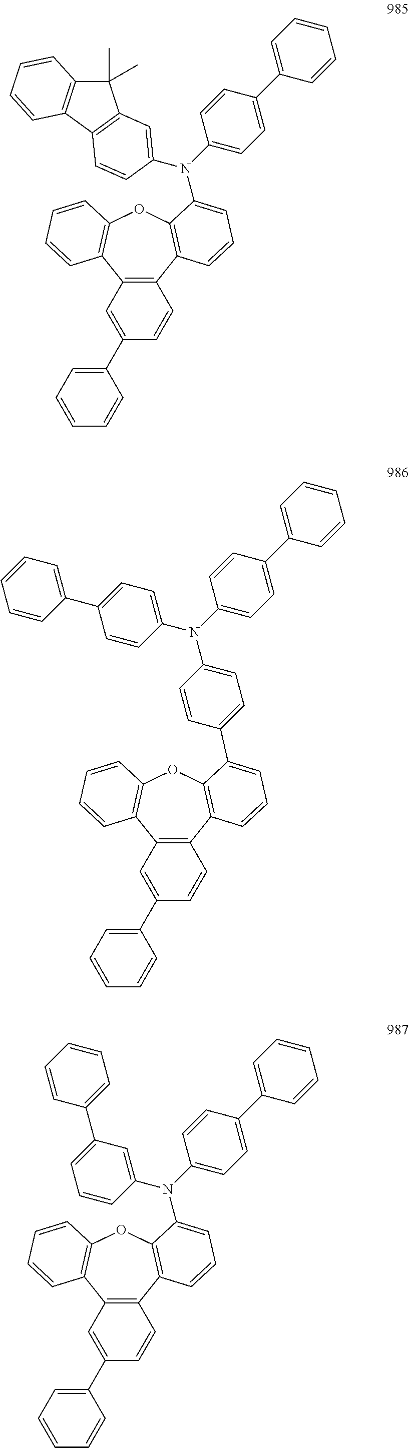 [SMILES: CC1(C)c2ccccc2-c2ccc(N(c3ccc(-c4ccccc4)cc3)c3cccc4c3Oc3ccccc3-c3cc(-c5ccccc5)ccc3-4)cc21 ; c1ccc(-c2ccc(N(c3ccc(-c4ccccc4)cc3)c3ccc(-c4cccc5c4Oc4ccccc4-c4cc(-c6ccccc6)ccc4-5)cc3)cc2)cc1 ; c1ccc(-c2ccc(N(c3cccc(-c4ccccc4)c3)c3cccc4c3Oc3ccccc3-c3cc(-c5ccccc5)ccc3-4)cc2)cc1]

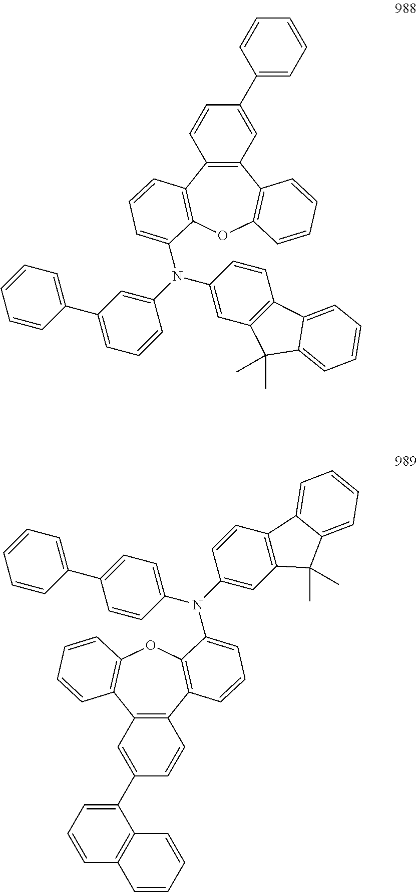 [SMILES: CC1(C)c2ccccc2-c2ccc(N(c3cccc(-c4ccccc4)c3)c3cccc4c3Oc3ccccc3-c3cc(-c5ccccc5)ccc3-4)cc21 ; CC1(C)c2ccccc2-c2ccc(N(c3ccc(-c4ccccc4)cc3)c3cccc4c3Oc3ccccc3-c3cc(-c5cccc6ccccc56)ccc3-4)cc21]

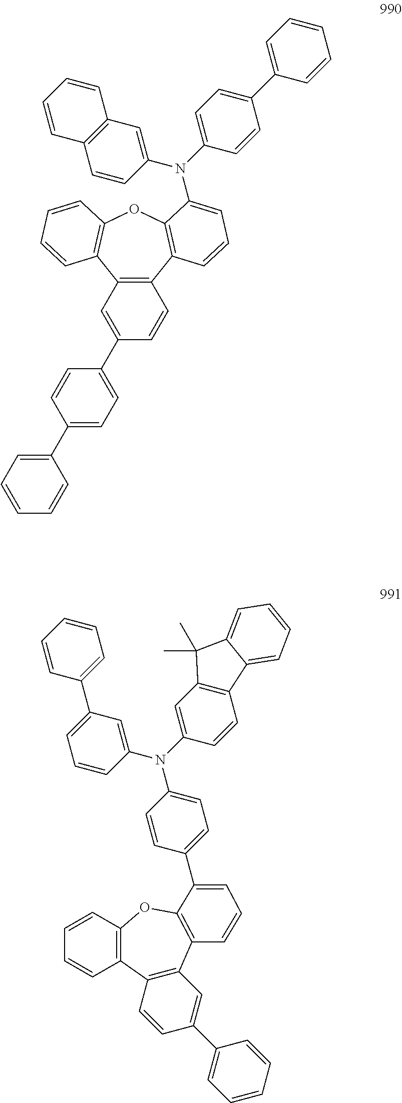 [SMILES: c1ccc(-c2ccc(-c3ccc4c(c3)-c3ccccc3Oc3c-4cccc3N(c3ccc(-c4ccccc4)cc3)c3ccc4ccccc4c3)cc2)cc1 ; CC1(C)c2ccccc2-c2ccc(N(c3ccc(-c4cccc5c4Oc4ccccc4-c4ccc(-c6ccccc6)cc4-5)cc3)c3cccc(-c4ccccc4)c3)cc21]

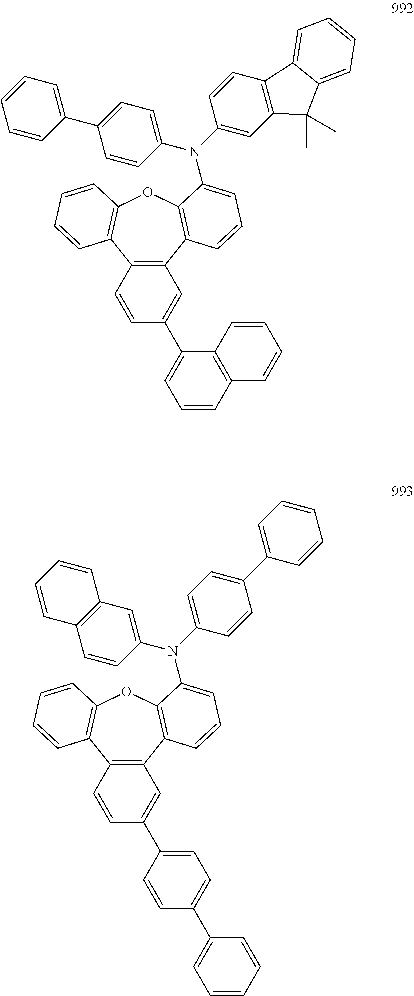 [SMILES: CC1(C)c2ccccc2-c2ccc(N(c3ccc(-c4ccccc4)cc3)c3cccc4c3Oc3ccccc3-c3ccc(-c5cccc6ccccc56)cc3-4)cc21 ; c1ccc(-c2ccc(-c3ccc4c(c3)-c3cccc(N(c5ccc(-c6ccccc6)cc5)c5ccc6ccccc6c5)c3Oc3ccccc3-4)cc2)cc1]

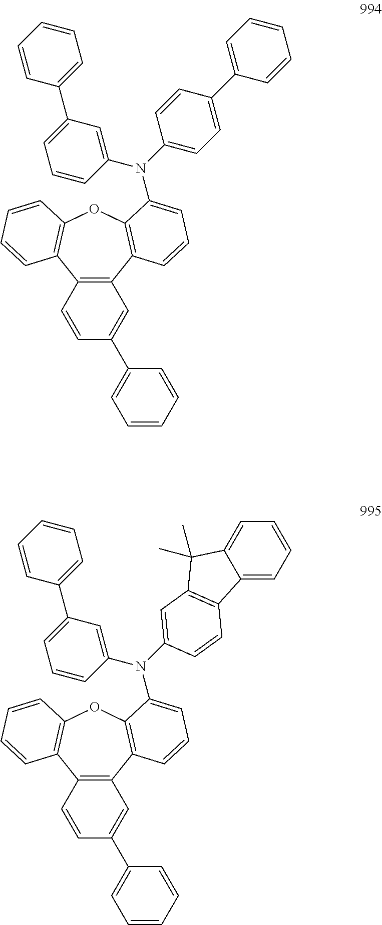 [SMILES: c1ccc(-c2ccc(N(c3cccc(-c4ccccc4)c3)c3cccc4c3Oc3ccccc3-c3ccc(-c5ccccc5)cc3-4)cc2)cc1 ; CC1(C)c2ccccc2-c2ccc(N(c3cccc(-c4ccccc4)c3)c3cccc4c3Oc3ccccc3-c3ccc(-c5ccccc5)cc3-4)cc21]

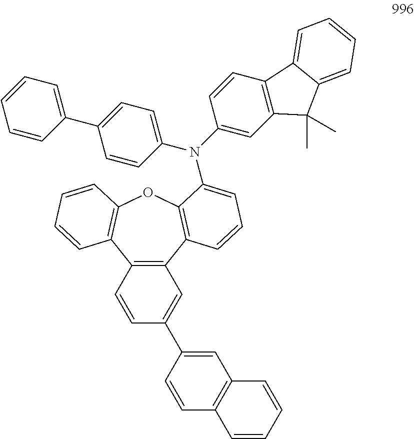 [SMILES: CC1(C)c2ccccc2-c2ccc(N(c3ccc(-c4ccccc4)cc3)c3cccc4c3Oc3ccccc3-c3ccc(-c5ccc6ccccc6c5)cc3-4)cc21]

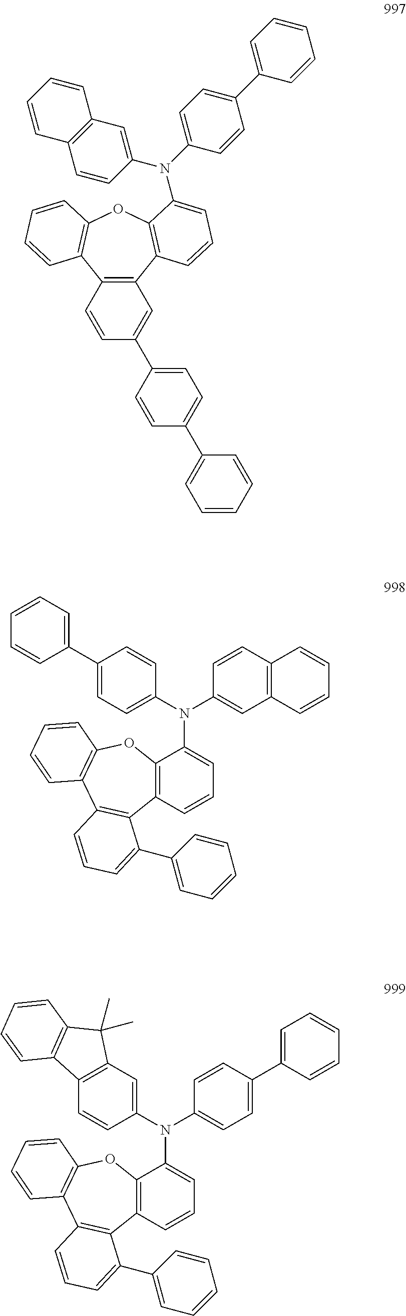 [SMILES: c1ccc(-c2ccc(-c3ccc4c(c3)-c3cccc(N(c5ccc(-c6ccccc6)cc5)c5ccc6ccccc6c5)c3Oc3ccccc3-4)cc2)cc1 ; c1ccc(-c2ccc(N(c3ccc4ccccc4c3)c3cccc4c3Oc3ccccc3-c3cccc(-c5ccccc5)c3-4)cc2)cc1 ; CC1(C)c2ccccc2-c2ccc(N(c3ccc(-c4ccccc4)cc3)c3cccc4c3Oc3ccccc3-c3cccc(-c5ccccc5)c3-4)cc21]

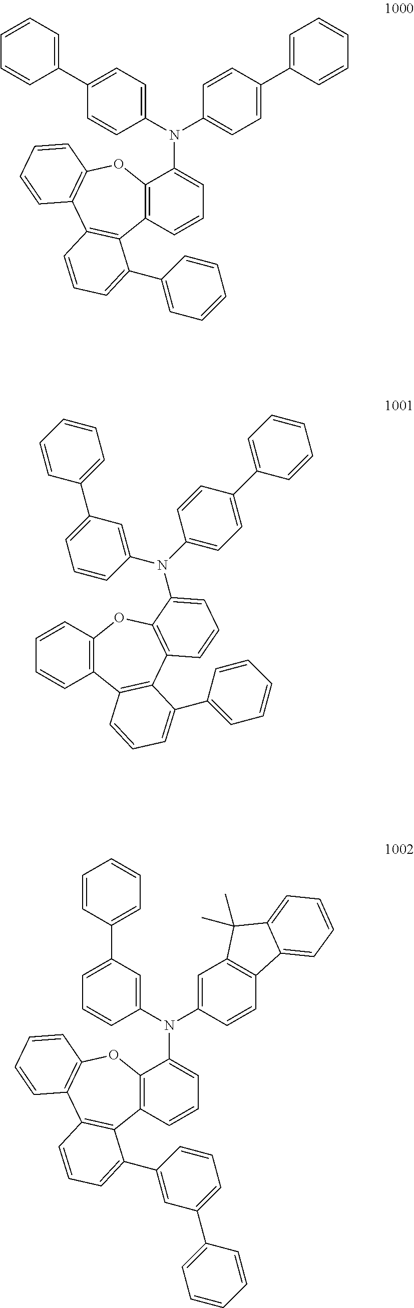 [SMILES: c1ccc(-c2ccc(N(c3ccc(-c4ccccc4)cc3)c3cccc4c3Oc3ccccc3-c3cccc(-c5ccccc5)c3-4)cc2)cc1 ; c1ccc(-c2ccc(N(c3cccc(-c4ccccc4)c3)c3cccc4c3Oc3ccccc3-c3cccc(-c5ccccc5)c3-4)cc2)cc1 ; CC1(C)c2ccccc2-c2ccc(N(c3cccc(-c4ccccc4)c3)c3cccc4c3Oc3ccccc3-c3cccc(-c5cccc(-c6ccccc6)c5)c3-4)cc21]

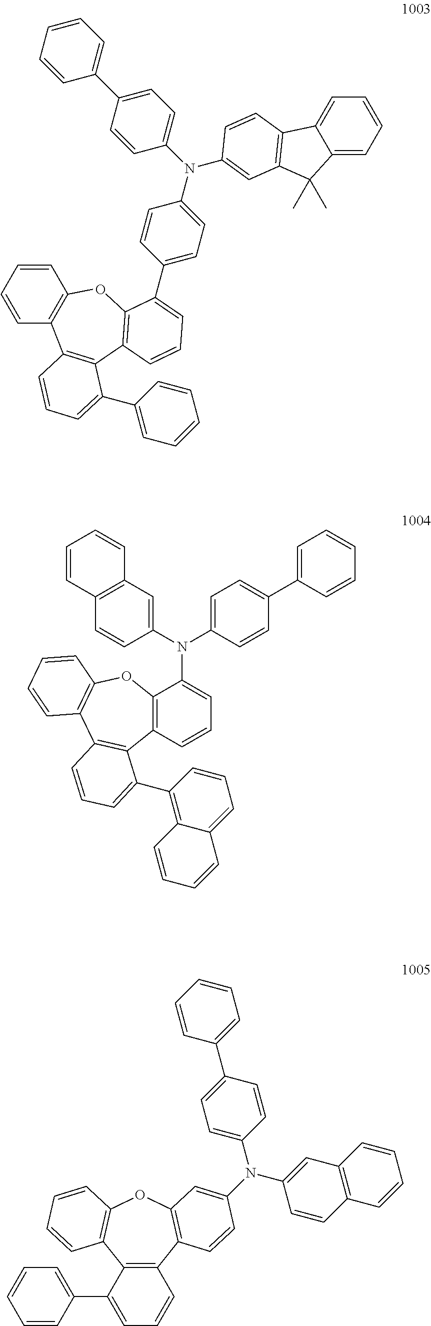 [SMILES: CC1(C)c2ccccc2-c2ccc(N(c3ccc(-c4ccccc4)cc3)c3ccc(-c4cccc5c4Oc4ccccc4-c4cccc(-c6ccccc6)c4-5)cc3)cc21 ; c1ccc(-c2ccc(N(c3ccc4ccccc4c3)c3cccc4c3Oc3ccccc3-c3cccc(-c5cccc6ccccc56)c3-4)cc2)cc1 ; c1ccc(-c2ccc(N(c3ccc4c(c3)Oc3ccccc3-c3c(-c5ccccc5)cccc3-4)c3ccc4ccccc4c3)cc2)cc1]

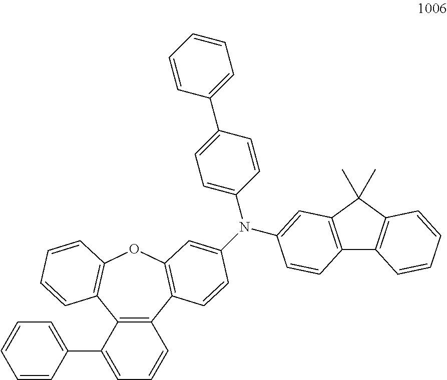 [SMILES: CC1(C)c2ccccc2-c2ccc(N(c3ccc(-c4ccccc4)cc3)c3ccc4c(c3)Oc3ccccc3-c3c(-c5ccccc5)cccc3-4)cc21]

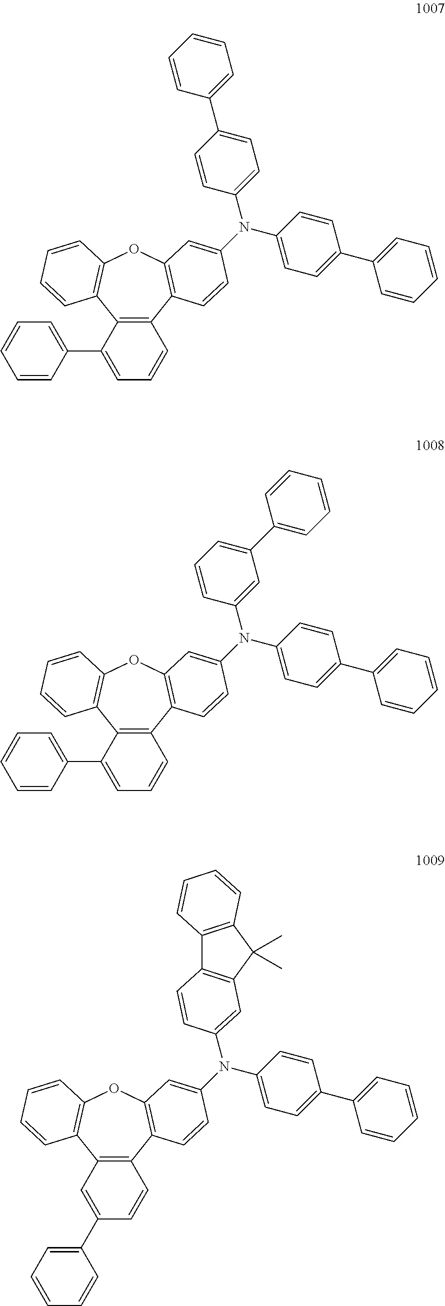 [SMILES: c1ccc(-c2ccc(N(c3ccc(-c4ccccc4)cc3)c3ccc4c(c3)Oc3ccccc3-c3c(-c5ccccc5)cccc3-4)cc2)cc1 ; c1ccc(-c2ccc(N(c3cccc(-c4ccccc4)c3)c3ccc4c(c3)Oc3ccccc3-c3c(-c5ccccc5)cccc3-4)cc2)cc1 ; CC1(C)c2ccccc2-c2ccc(N(c3ccc(-c4ccccc4)cc3)c3ccc4c(c3)Oc3ccccc3-c3cc(-c5ccccc5)ccc3-4)cc21]

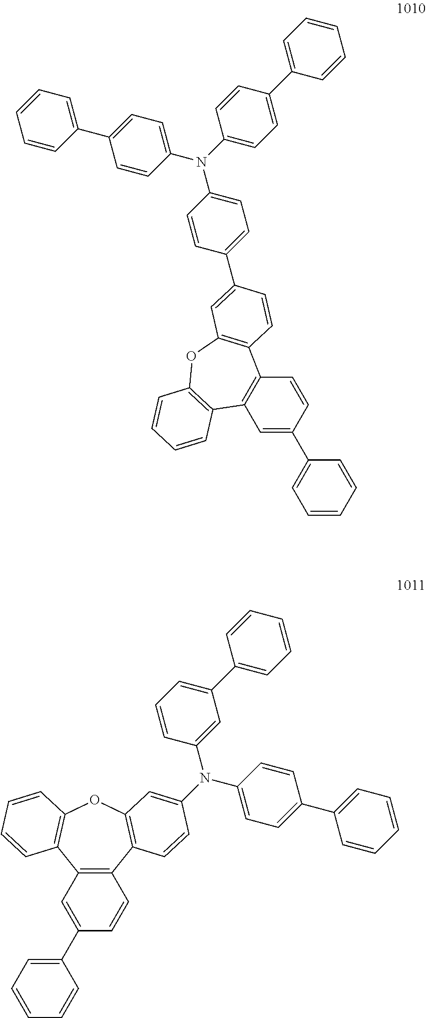 [SMILES: c1ccc(-c2ccc(N(c3ccc(-c4ccccc4)cc3)c3ccc(-c4ccc5c(c4)Oc4ccccc4-c4cc(-c6ccccc6)ccc4-5)cc3)cc2)cc1 ; c1ccc(-c2ccc(N(c3cccc(-c4ccccc4)c3)c3ccc4c(c3)Oc3ccccc3-c3cc(-c5ccccc5)ccc3-4)cc2)cc1]

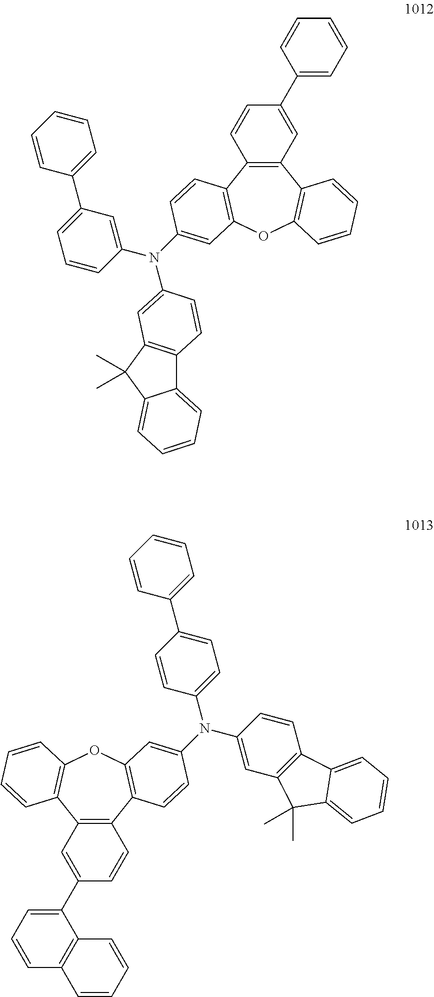 [SMILES: CC1(C)c2ccccc2-c2ccc(N(c3cccc(-c4ccccc4)c3)c3ccc4c(c3)Oc3ccccc3-c3cc(-c5ccccc5)ccc3-4)cc21 ; CC1(C)c2ccccc2-c2ccc(N(c3ccc(-c4ccccc4)cc3)c3ccc4c(c3)Oc3ccccc3-c3cc(-c5cccc6ccccc56)ccc3-4)cc21]

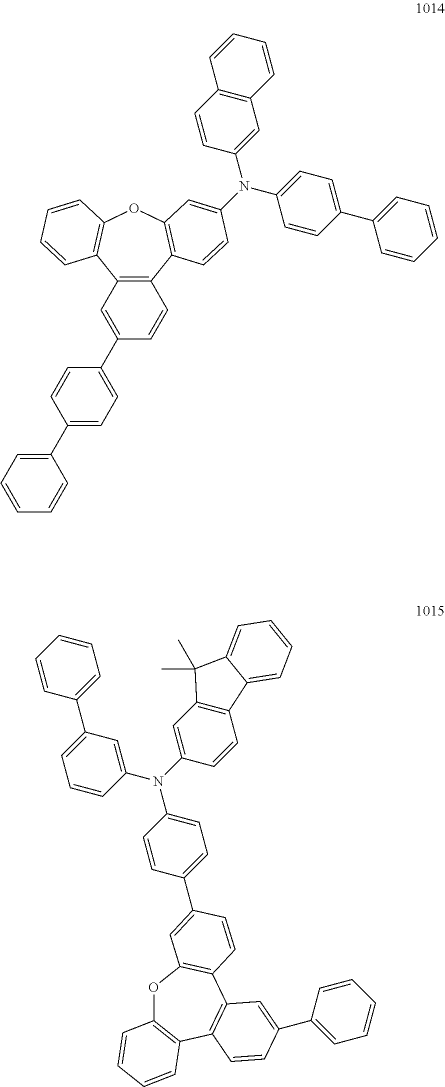 [SMILES: c1ccc(-c2ccc(-c3ccc4c(c3)-c3ccccc3Oc3cc(N(c5ccc(-c6ccccc6)cc5)c5ccc6ccccc6c5)ccc3-4)cc2)cc1 ; CC1(C)c2ccccc2-c2ccc(N(c3ccc(-c4ccc5c(c4)Oc4ccccc4-c4ccc(-c6ccccc6)cc4-5)cc3)c3cccc(-c4ccccc4)c3)cc21]

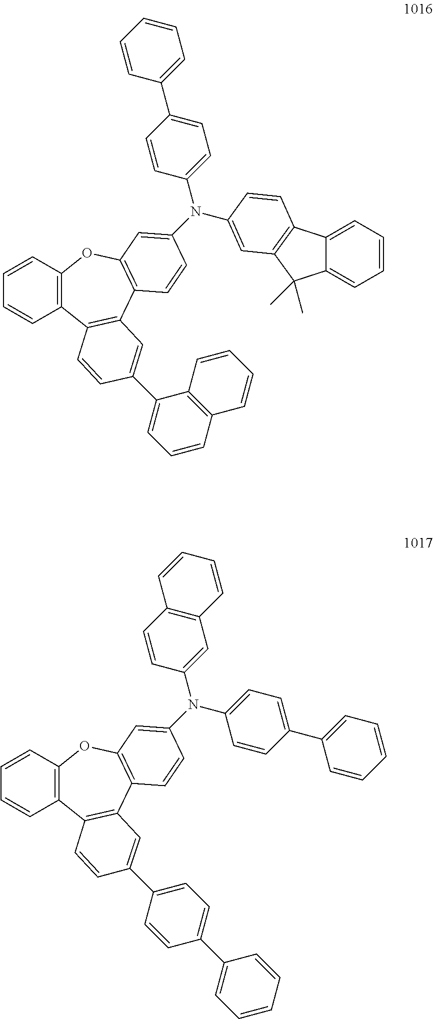 [SMILES: CC1(C)c2ccccc2-c2ccc(N(c3ccc(-c4ccccc4)cc3)c3ccc4c(c3)Oc3ccccc3-c3ccc(-c5cccc6ccccc56)cc3-4)cc21 ; c1ccc(-c2ccc(-c3ccc4c(c3)-c3ccc(N(c5ccc(-c6ccccc6)cc5)c5ccc6ccccc6c5)cc3Oc3ccccc3-4)cc2)cc1]

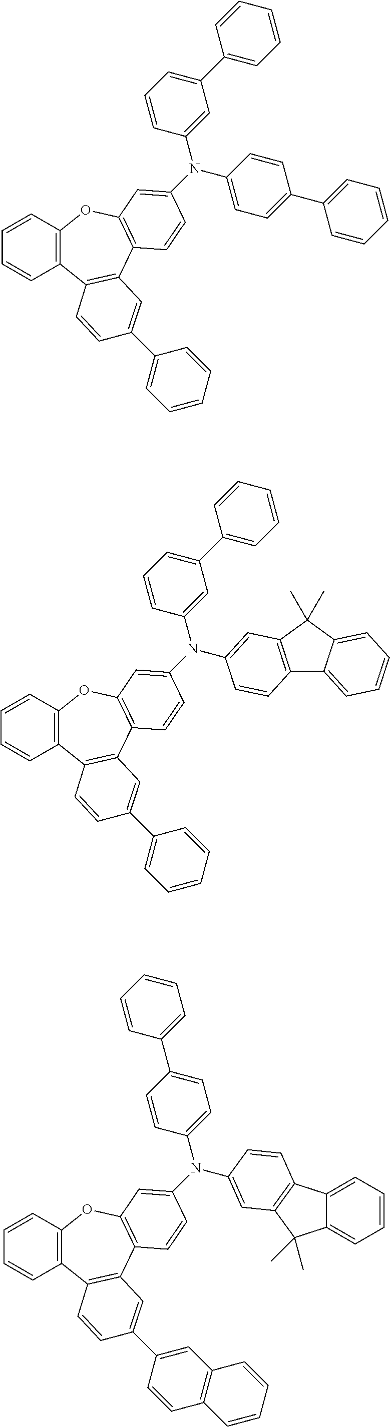 [SMILES: c1ccc(-c2ccc(N(c3cccc(-c4ccccc4)c3)c3ccc4c(c3)Oc3ccccc3-c3ccc(-c5ccccc5)cc3-4)cc2)cc1 ; CC1(C)c2ccccc2-c2ccc(N(c3cccc(-c4ccccc4)c3)c3ccc4c(c3)Oc3ccccc3-c3ccc(-c5ccccc5)cc3-4)cc21 ; CC1(C)c2ccccc2-c2ccc(N(c3ccc(-c4ccccc4)cc3)c3ccc4c(c3)Oc3ccccc3-c3ccc(-c5ccc6ccccc6c5)cc3-4)cc21]

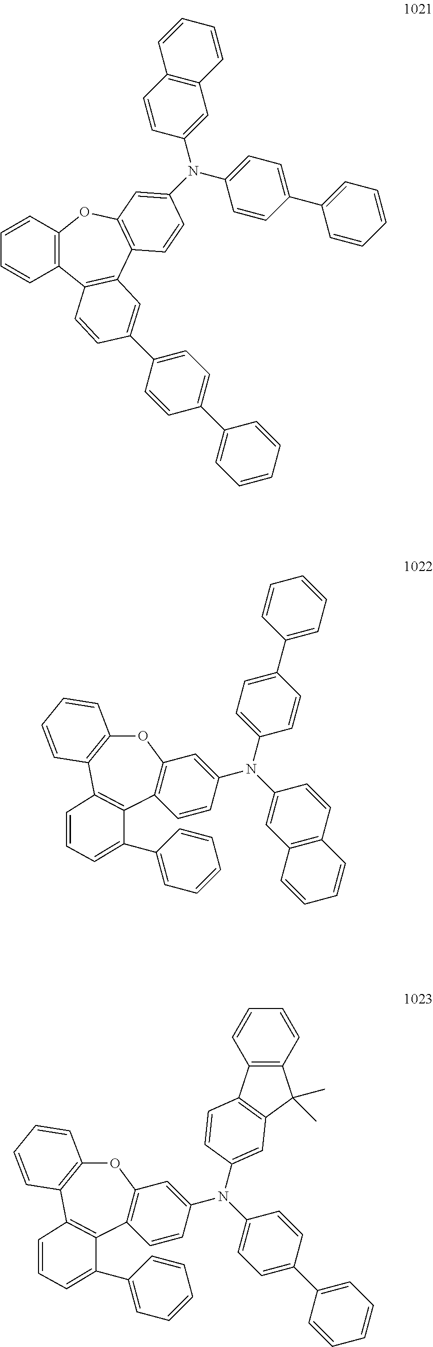 [SMILES: c1ccc(-c2ccc(-c3ccc4c(c3)-c3ccc(N(c5ccc(-c6ccccc6)cc5)c5ccc6ccccc6c5)cc3Oc3ccccc3-4)cc2)cc1 ; c1ccc(-c2ccc(N(c3ccc4c(c3)Oc3ccccc3-c3cccc(-c5ccccc5)c3-4)c3ccc4ccccc4c3)cc2)cc1 ; CC1(C)c2ccccc2-c2ccc(N(c3ccc(-c4ccccc4)cc3)c3ccc4c(c3)Oc3ccccc3-c3cccc(-c5ccccc5)c3-4)cc21]

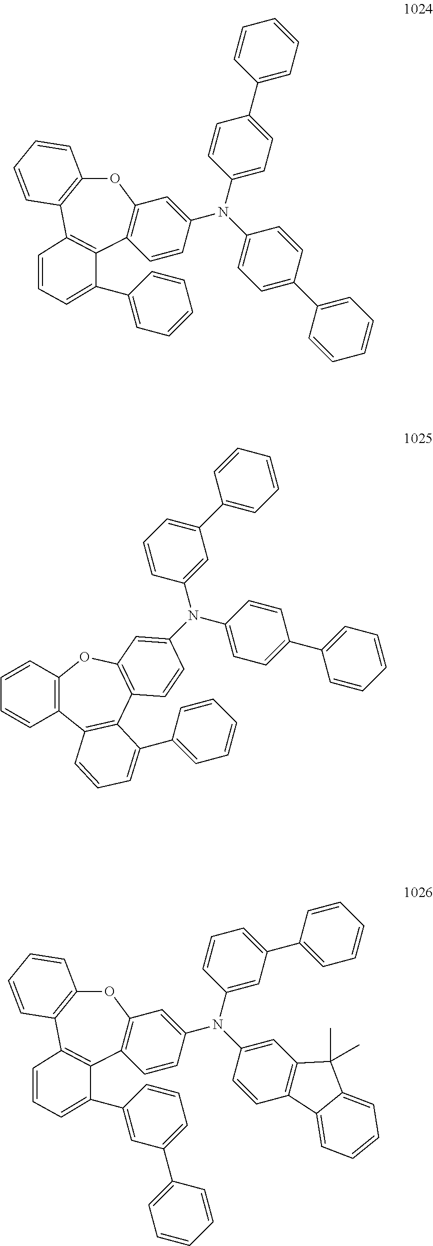 [SMILES: c1ccc(-c2ccc(N(c3ccc(-c4ccccc4)cc3)c3ccc4c(c3)Oc3ccccc3-c3cccc(-c5ccccc5)c3-4)cc2)cc1 ; c1ccc(-c2ccc(N(c3cccc(-c4ccccc4)c3)c3ccc4c(c3)Oc3ccccc3-c3cccc(-c5ccccc5)c3-4)cc2)cc1 ; CC1(C)c2ccccc2-c2ccc(N(c3cccc(-c4ccccc4)c3)c3ccc4c(c3)Oc3ccccc3-c3cccc(-c5cccc(-c6ccccc6)c5)c3-4)cc21]

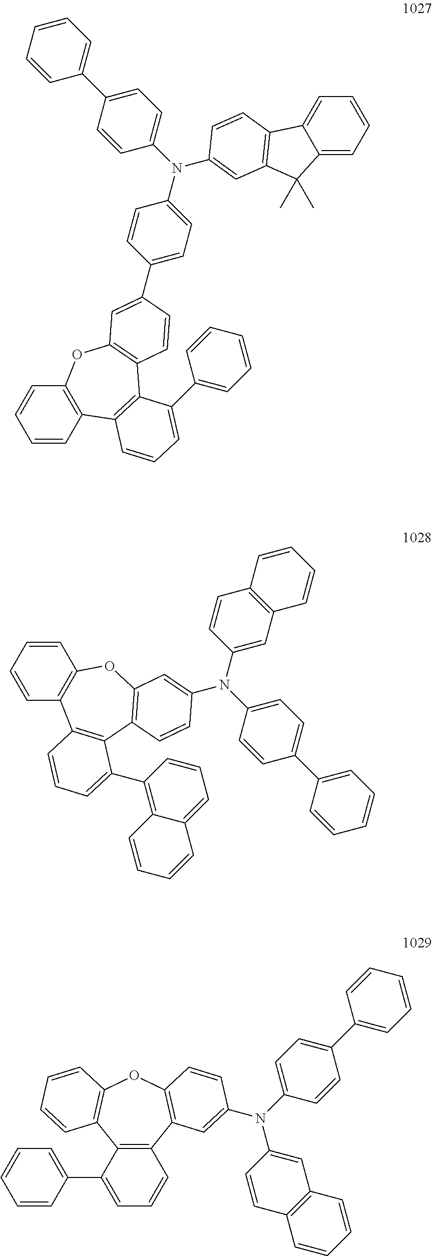 [SMILES: CC1(C)c2ccccc2-c2ccc(N(c3ccc(-c4ccccc4)cc3)c3ccc(-c4ccc5c(c4)Oc4ccccc4-c4cccc(-c6ccccc6)c4-5)cc3)cc21 ; c1ccc(-c2ccc(N(c3ccc4c(c3)Oc3ccccc3-c3cccc(-c5cccc6ccccc56)c3-4)c3ccc4ccccc4c3)cc2)cc1 ; c1ccc(-c2ccc(N(c3ccc4c(c3)-c3cccc(-c5ccccc5)c3-c3ccccc3O4)c3ccc4ccccc4c3)cc2)cc1]

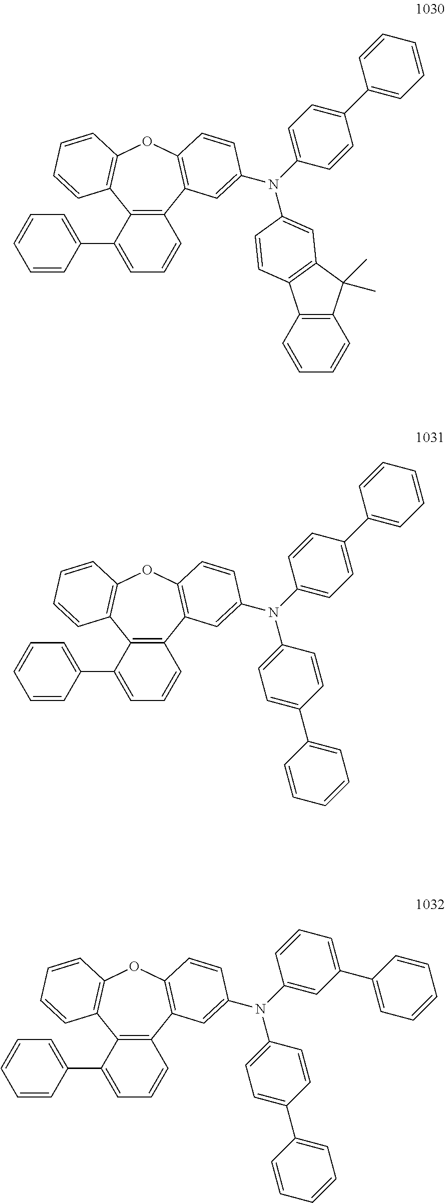 [SMILES: CC1(C)c2ccccc2-c2ccc(N(c3ccc(-c4ccccc4)cc3)c3ccc4c(c3)-c3cccc(-c5ccccc5)c3-c3ccccc3O4)cc21 ; c1ccc(-c2ccc(N(c3ccc(-c4ccccc4)cc3)c3ccc4c(c3)-c3cccc(-c5ccccc5)c3-c3ccccc3O4)cc2)cc1 ; c1ccc(-c2ccc(N(c3cccc(-c4ccccc4)c3)c3ccc4c(c3)-c3cccc(-c5ccccc5)c3-c3ccccc3O4)cc2)cc1]

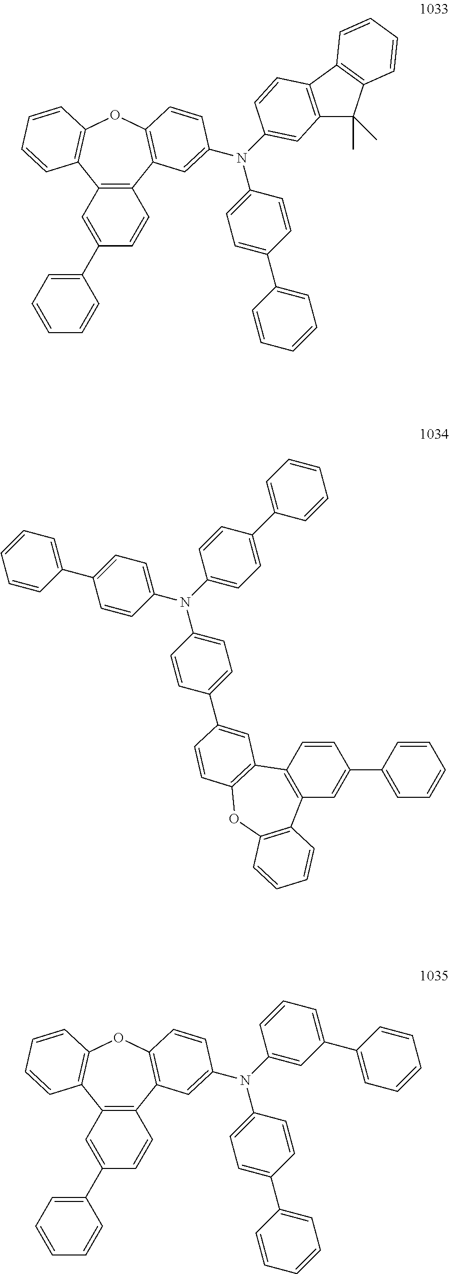 [SMILES: CC1(C)c2ccccc2-c2ccc(N(c3ccc(-c4ccccc4)cc3)c3ccc4c(c3)-c3ccc(-c5ccccc5)cc3-c3ccccc3O4)cc21 ; c1ccc(-c2ccc(N(c3ccc(-c4ccccc4)cc3)c3ccc(-c4ccc5c(c4)-c4ccc(-c6ccccc6)cc4-c4ccccc4O5)cc3)cc2)cc1 ; c1ccc(-c2ccc(N(c3cccc(-c4ccccc4)c3)c3ccc4c(c3)-c3ccc(-c5ccccc5)cc3-c3ccccc3O4)cc2)cc1]

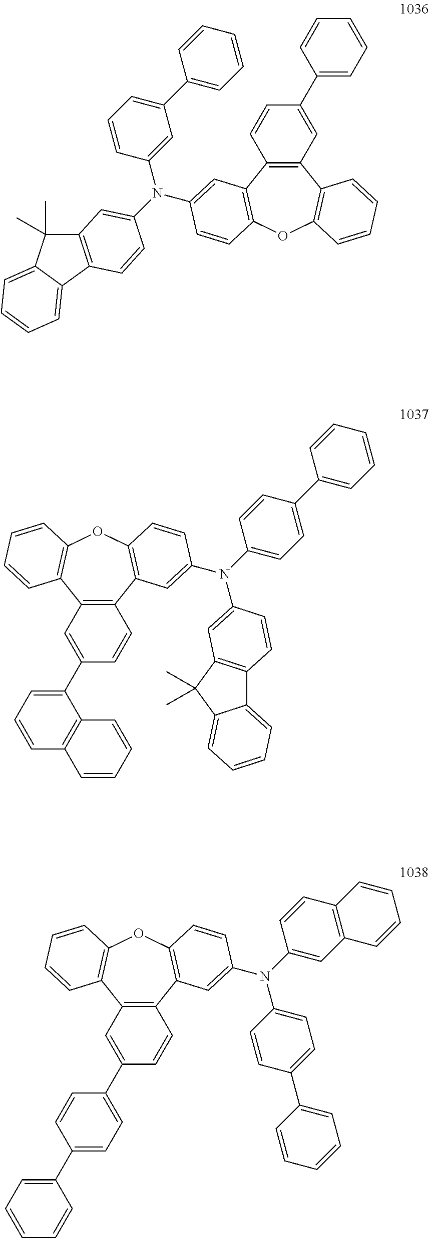 [SMILES: CC1(C)c2ccccc2-c2ccc(N(c3cccc(-c4ccccc4)c3)c3ccc4c(c3)-c3ccc(-c5ccccc5)cc3-c3ccccc3O4)cc21 ; CC1(C)c2ccccc2-c2ccc(N(c3ccc(-c4ccccc4)cc3)c3ccc4c(c3)-c3ccc(-c5cccc6ccccc56)cc3-c3ccccc3O4)cc21 ; c1ccc(-c2ccc(-c3ccc4c(c3)-c3ccccc3Oc3ccc(N(c5ccc(-c6ccccc6)cc5)c5ccc6ccccc6c5)cc3-4)cc2)cc1]

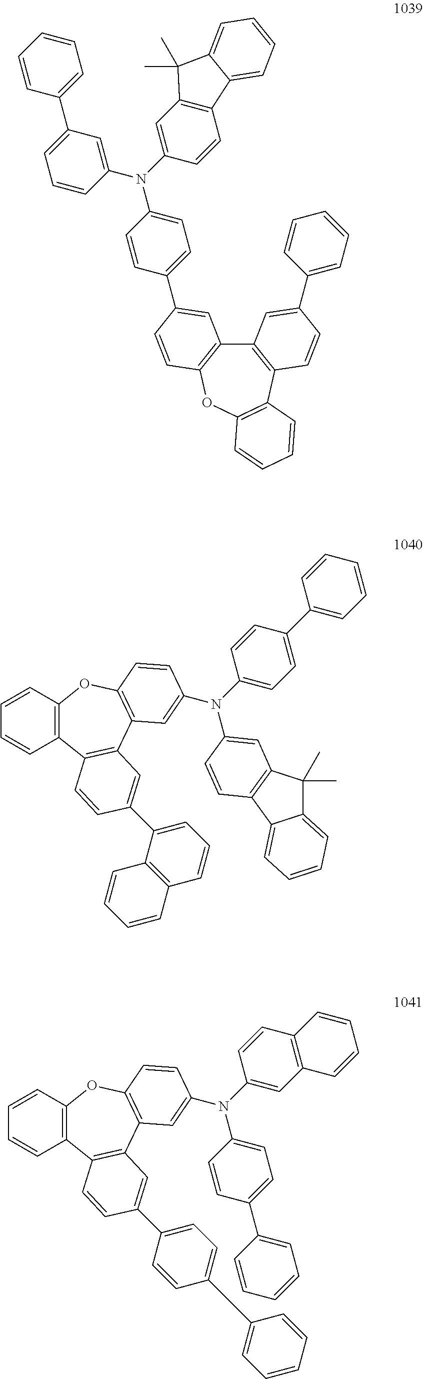 [SMILES: CC1(C)c2ccccc2-c2ccc(N(c3ccc(-c4ccc5c(c4)-c4cc(-c6ccccc6)ccc4-c4ccccc4O5)cc3)c3cccc(-c4ccccc4)c3)cc21 ; CC1(C)c2ccccc2-c2ccc(N(c3ccc(-c4ccccc4)cc3)c3ccc4c(c3)-c3cc(-c5cccc6ccccc56)ccc3-c3ccccc3O4)cc21 ; c1ccc(-c2ccc(-c3ccc4c(c3)-c3cc(N(c5ccc(-c6ccccc6)cc5)c5ccc6ccccc6c5)ccc3Oc3ccccc3-4)cc2)cc1]

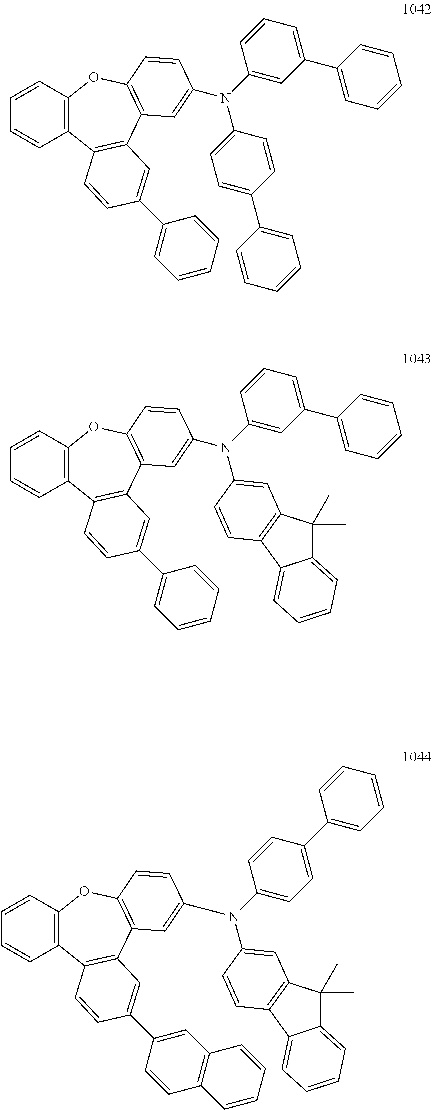 [SMILES: c1ccc(-c2ccc(N(c3cccc(-c4ccccc4)c3)c3ccc4c(c3)-c3cc(-c5ccccc5)ccc3-c3ccccc3O4)cc2)cc1 ; CC1(C)c2ccccc2-c2ccc(N(c3cccc(-c4ccccc4)c3)c3ccc4c(c3)-c3cc(-c5ccccc5)ccc3-c3ccccc3O4)cc21 ; CC1(C)c2ccccc2-c2ccc(N(c3ccc(-c4ccccc4)cc3)c3ccc4c(c3)-c3cc(-c5ccc6ccccc6c5)ccc3-c3ccccc3O4)cc21]

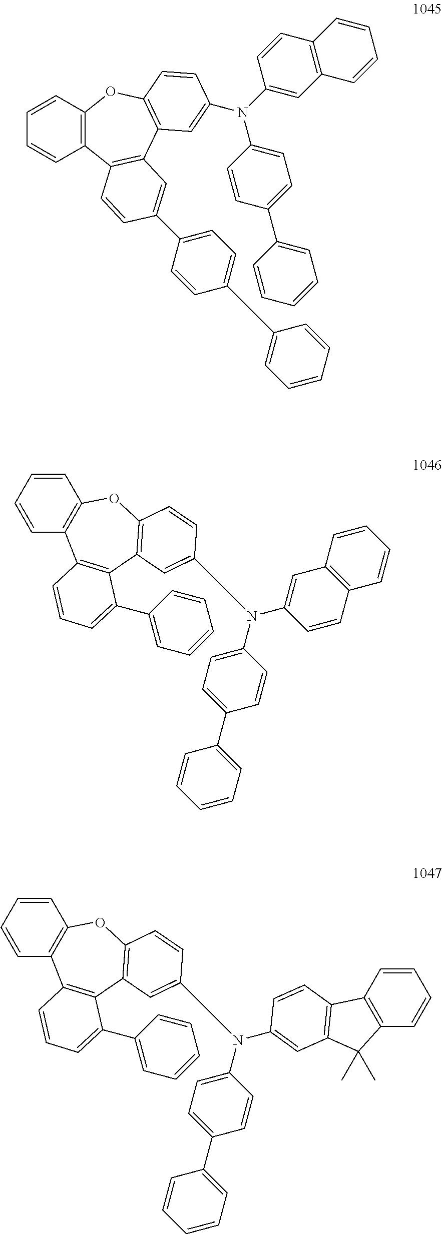 [SMILES: c1ccc(-c2ccc(-c3ccc4c(c3)-c3cc(N(c5ccc(-c6ccccc6)cc5)c5ccc6ccccc6c5)ccc3Oc3ccccc3-4)cc2)cc1 ; c1ccc(-c2ccc(N(c3ccc4c(c3)-c3c(-c5ccccc5)cccc3-c3ccccc3O4)c3ccc4ccccc4c3)cc2)cc1 ; CC1(C)c2ccccc2-c2ccc(N(c3ccc(-c4ccccc4)cc3)c3ccc4c(c3)-c3c(-c5ccccc5)cccc3-c3ccccc3O4)cc21]

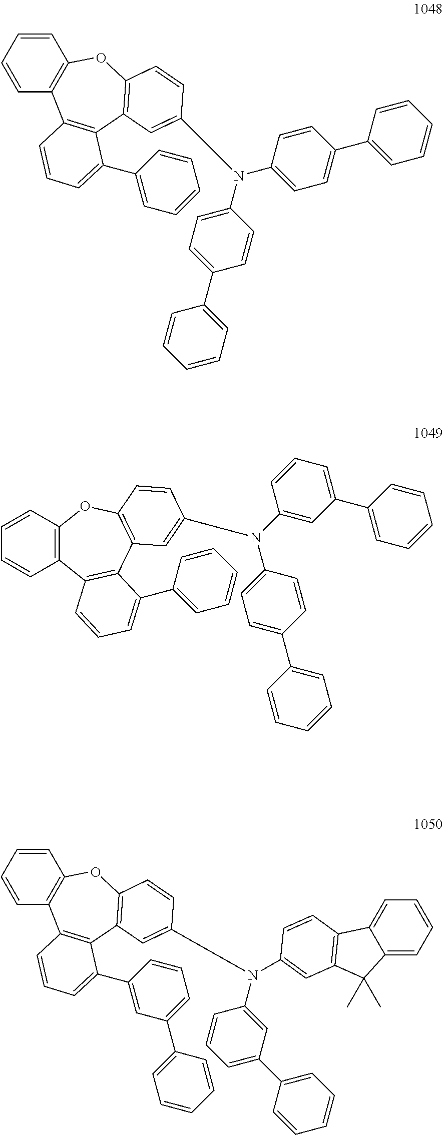 [SMILES: c1ccc(-c2ccc(N(c3ccc(-c4ccccc4)cc3)c3ccc4c(c3)-c3c(-c5ccccc5)cccc3-c3ccccc3O4)cc2)cc1 ; c1ccc(-c2ccc(N(c3cccc(-c4ccccc4)c3)c3ccc4c(c3)-c3c(-c5ccccc5)cccc3-c3ccccc3O4)cc2)cc1 ; CC1(C)c2ccccc2-c2ccc(N(c3cccc(-c4ccccc4)c3)c3ccc4c(c3)-c3c(-c5cccc(-c6ccccc6)c5)cccc3-c3ccccc3O4)cc21]

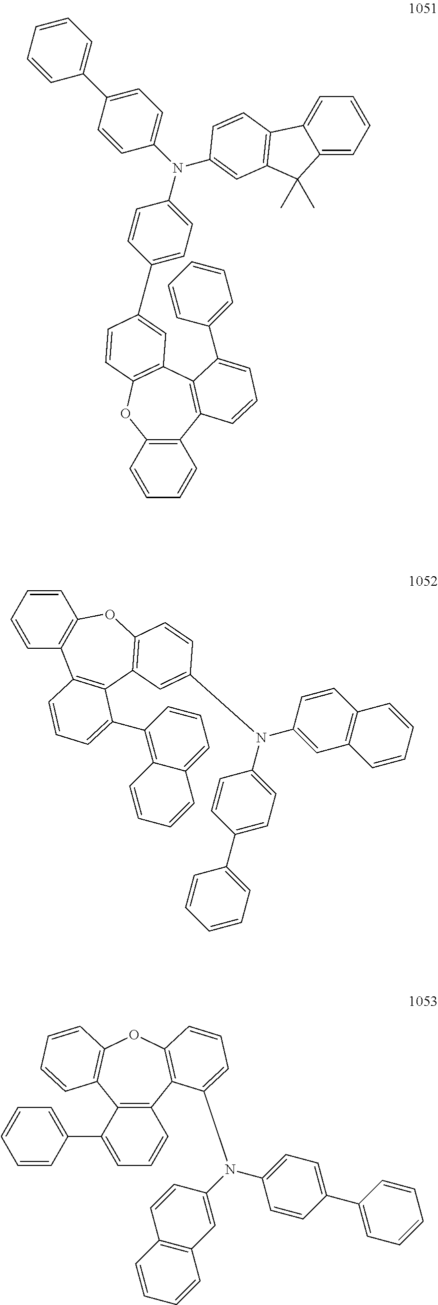 [SMILES: CC1(C)c2ccccc2-c2ccc(N(c3ccc(-c4ccccc4)cc3)c3ccc(-c4ccc5c(c4)-c4c(-c6ccccc6)cccc4-c4ccccc4O5)cc3)cc21 ; c1ccc(-c2ccc(N(c3ccc4c(c3)-c3c(cccc3-c3cccc5ccccc35)-c3ccccc3O4)c3ccc4ccccc4c3)cc2)cc1 ; c1ccc(-c2ccc(N(c3ccc4ccccc4c3)c3cccc4c3-c3cccc(-c5ccccc5)c3-c3ccccc3O4)cc2)cc1]

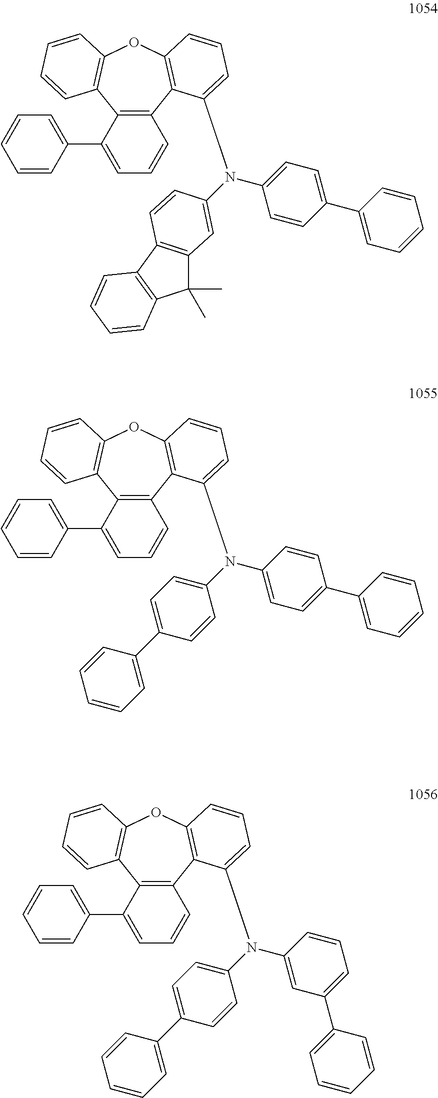 [SMILES: CC1(C)c2ccccc2-c2ccc(N(c3ccc(-c4ccccc4)cc3)c3cccc4c3-c3cccc(-c5ccccc5)c3-c3ccccc3O4)cc21 ; c1ccc(-c2ccc(N(c3ccc(-c4ccccc4)cc3)c3cccc4c3-c3cccc(-c5ccccc5)c3-c3ccccc3O4)cc2)cc1 ; c1ccc(-c2ccc(N(c3cccc(-c4ccccc4)c3)c3cccc4c3-c3cccc(-c5ccccc5)c3-c3ccccc3O4)cc2)cc1]

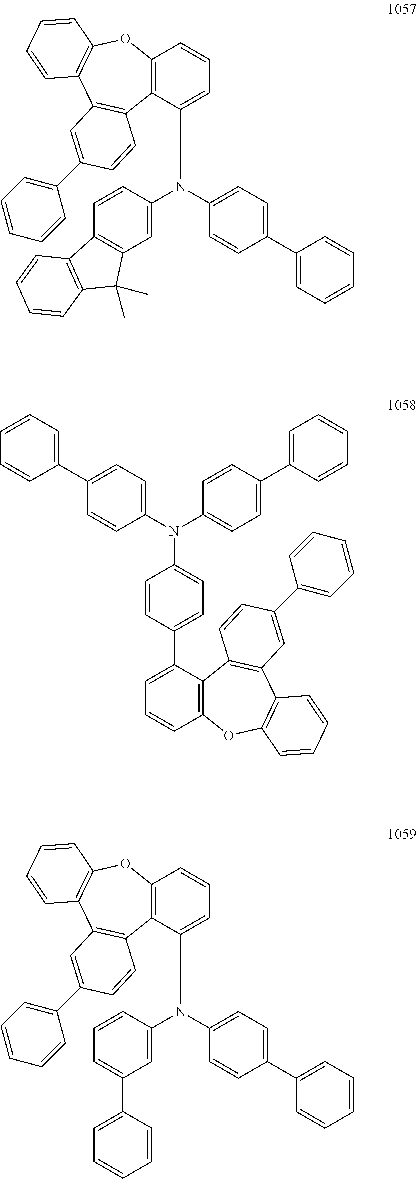 [SMILES: CC1(C)c2ccccc2-c2ccc(N(c3ccc(-c4ccccc4)cc3)c3cccc4c3-c3ccc(-c5ccccc5)cc3-c3ccccc3O4)cc21 ; c1ccc(-c2ccc(N(c3ccc(-c4ccccc4)cc3)c3ccc(-c4cccc5c4-c4ccc(-c6ccccc6)cc4-c4ccccc4O5)cc3)cc2)cc1 ; c1ccc(-c2ccc(N(c3cccc(-c4ccccc4)c3)c3cccc4c3-c3ccc(-c5ccccc5)cc3-c3ccccc3O4)cc2)cc1]

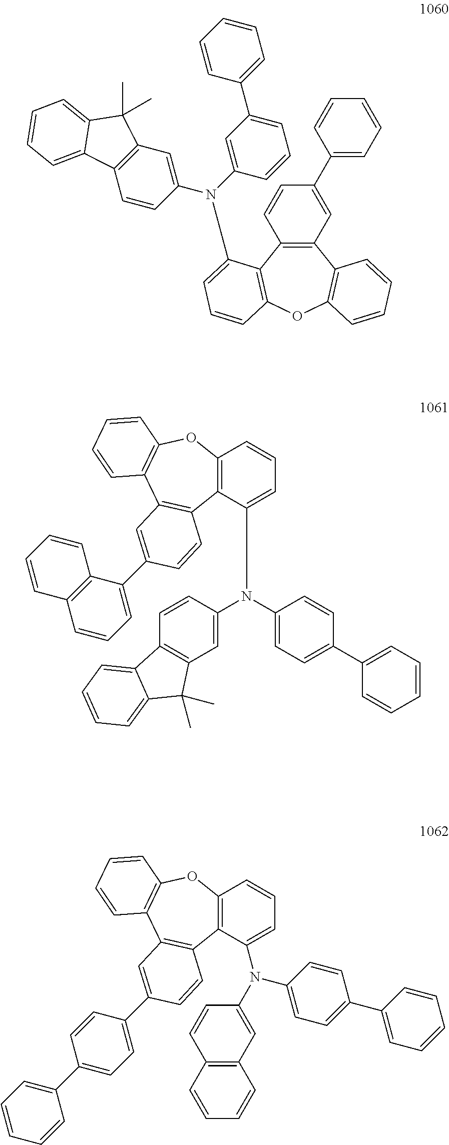 [SMILES: CC1(C)c2ccccc2-c2ccc(N(c3cccc(-c4ccccc4)c3)c3cccc4c3-c3ccc(-c5ccccc5)cc3-c3ccccc3O4)cc21 ; CC1(C)c2ccccc2-c2ccc(N(c3ccc(-c4ccccc4)cc3)c3cccc4c3-c3ccc(-c5cccc6ccccc56)cc3-c3ccccc3O4)cc21 ; c1ccc(-c2ccc(-c3ccc4c(c3)-c3ccccc3Oc3cccc(N(c5ccc(-c6ccccc6)cc5)c5ccc6ccccc6c5)c3-4)cc2)cc1]

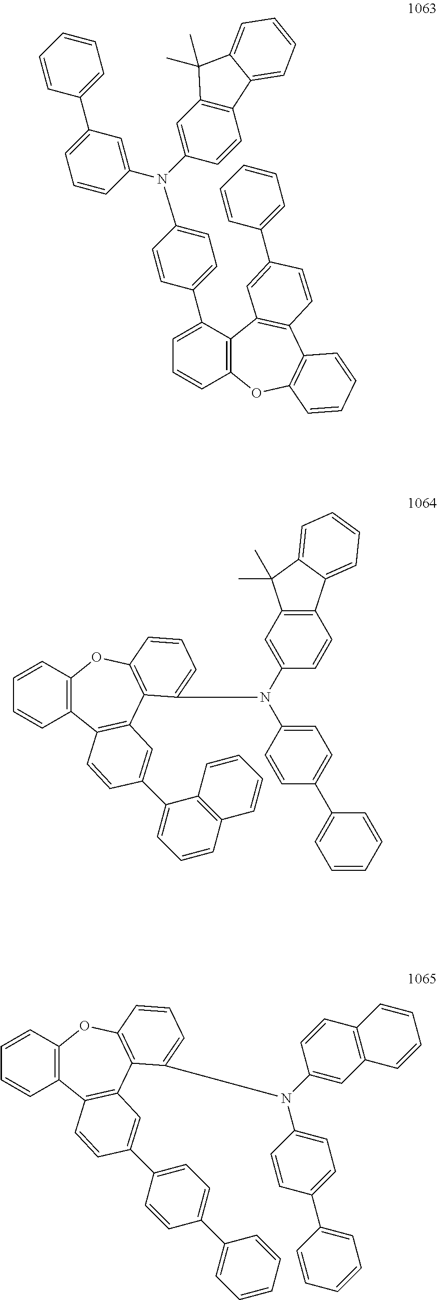 [SMILES: CC1(C)c2ccccc2-c2ccc(N(c3ccc(-c4cccc5c4-c4cc(-c6ccccc6)ccc4-c4ccccc4O5)cc3)c3cccc(-c4ccccc4)c3)cc21 ; CC1(C)c2ccccc2-c2ccc(N(c3ccc(-c4ccccc4)cc3)c3cccc4c3-c3cc(-c5cccc6ccccc56)ccc3-c3ccccc3O4)cc21 ; c1ccc(-c2ccc(-c3ccc4c(c3)-c3c(cccc3N(c3ccc(-c5ccccc5)cc3)c3ccc5ccccc5c3)Oc3ccccc3-4)cc2)cc1]

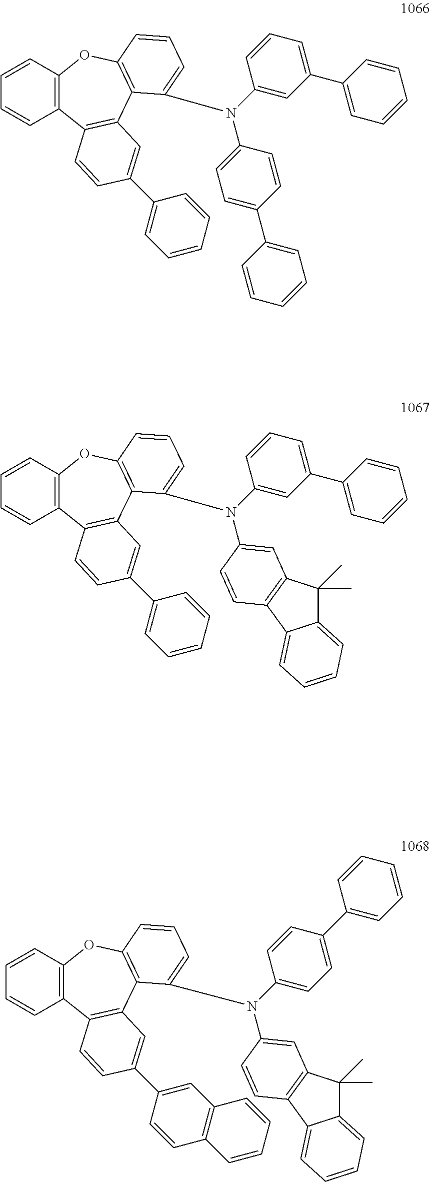 [SMILES: c1ccc(-c2ccc(N(c3cccc(-c4ccccc4)c3)c3cccc4c3-c3cc(-c5ccccc5)ccc3-c3ccccc3O4)cc2)cc1 ; CC1(C)c2ccccc2-c2ccc(N(c3cccc(-c4ccccc4)c3)c3cccc4c3-c3cc(-c5ccccc5)ccc3-c3ccccc3O4)cc21 ; CC1(C)c2ccccc2-c2ccc(N(c3ccc(-c4ccccc4)cc3)c3cccc4c3-c3cc(-c5ccc6ccccc6c5)ccc3-c3ccccc3O4)cc21]

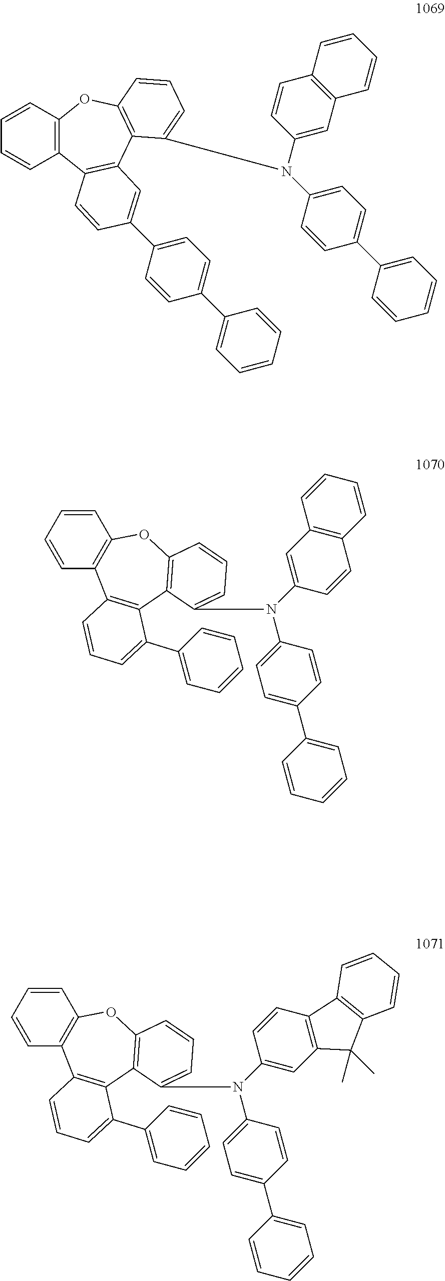 [SMILES: c1ccc(-c2ccc(-c3ccc4c(c3)-c3c(cccc3N(c3ccc(-c5ccccc5)cc3)c3ccc5ccccc5c3)Oc3ccccc3-4)cc2)cc1 ; c1ccc(-c2ccc(N(c3ccc4ccccc4c3)c3cccc4c3-c3c(-c5ccccc5)cccc3-c3ccccc3O4)cc2)cc1 ; CC1(C)c2ccccc2-c2ccc(N(c3ccc(-c4ccccc4)cc3)c3cccc4c3-c3c(-c5ccccc5)cccc3-c3ccccc3O4)cc21]

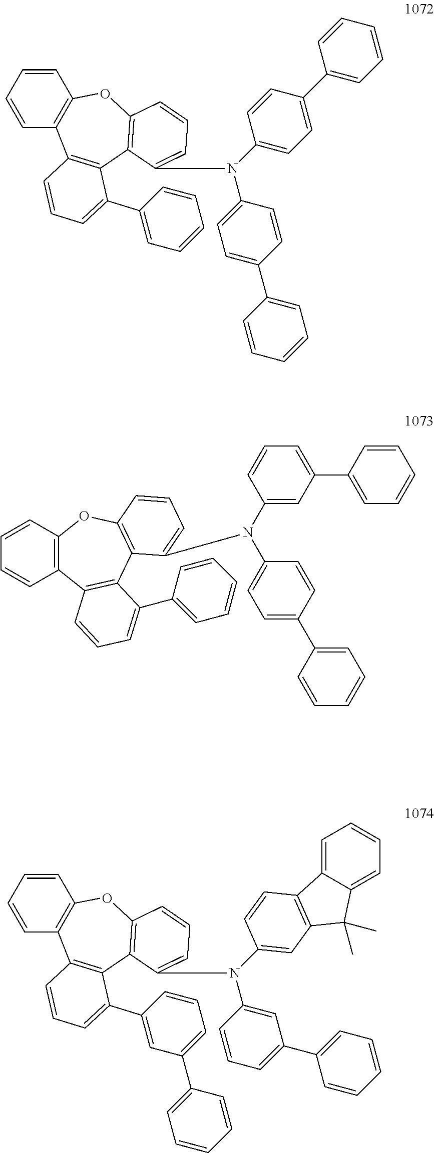 [SMILES: c1ccc(-c2ccc(N(c3ccc(-c4ccccc4)cc3)c3cccc4c3-c3c(-c5ccccc5)cccc3-c3ccccc3O4)cc2)cc1 ; c1ccc(-c2ccc(N(c3cccc(-c4ccccc4)c3)c3cccc4c3-c3c(-c5ccccc5)cccc3-c3ccccc3O4)cc2)cc1 ; CC1(C)c2ccccc2-c2ccc(N(c3cccc(-c4ccccc4)c3)c3cccc4c3-c3c(-c5cccc(-c6ccccc6)c5)cccc3-c3ccccc3O4)cc21]

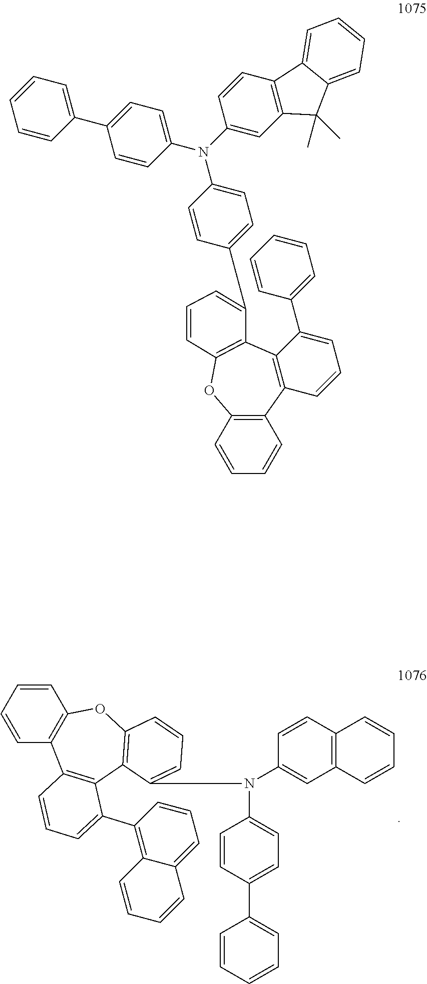 [SMILES: CC1(C)c2ccccc2-c2ccc(N(c3ccc(-c4ccccc4)cc3)c3ccc(-c4cccc5c4-c4c(-c6ccccc6)cccc4-c4ccccc4O5)cc3)cc21 ; c1ccc(-c2ccc(N(c3ccc4ccccc4c3)c3cccc4c3-c3c(cccc3-c3cccc5ccccc35)-c3ccccc3O4)cc2)cc1]

10. An organic light emitting device comprising:
a first electrode;
a second electrode provided to face the first electrode; and
one or more organic material layers provided between the first electrode and the second electrode,
wherein at least one of the one or more layers of the organic material layers comprises the heterocyclic compound according to claim 1.

11. The organic light emitting device according to claim 10, wherein the organic material layers comprise a hole transport layer, and the hole transport layer comprise the heterocyclic compound.

12. The organic light emitting device according to claim 10, wherein the organic material layers comprise an electron blocking layer, and the electron blocking layer comprise the heterocyclic compound.

13. The organic light emitting device according to claim 10, wherein the organic material layers comprise an electron injection layer, a hole injection layer, an electron transport layer, or a hole blocking layer, and the electron injection layer, the hole injection layer, the electron transport layer, or the hole blocking layer comprises the heterocyclic compound.

14. The organic light emitting device according to claim 10, wherein the organic light emitting device further comprises one or more selected from the group consisting of a light emitting layer, a hole injection layer, a hole transport layer, a hole blocking layer, an electron injection layer, an electron transport layer, an electron blocking layer, and a hole blocking layer.
